(19) United States
(12) Patent Application Publication
(10) Pub. No.: US 2026/0035425 A1
(43) Pub. Date: Feb. 5, 2026

(54) MEMBRANE-ANCHORED CYTOKINES, ENGINEERED IMMUNE CELLS, AND USES THEREOF

(71) Applicant: Chengdu Ucello Biotechnology Co., Limited, Chengdu (CN)

(72) Inventors: Qiang ZOU, Chengdu (CN); Hanmei OUYANG, Chengdu (CN); Hai SUN, Chengdu (CN); Xiao YUAN, Chengdu (CN); Qipin XU, Chengdu (CN); Quanming AN, Chengdu (CN); Jieyun JI, Chengdu (CN); Zhaoyan WANG, Chengdu (CN)

(21) Appl. No.: 19/264,479

(22) Filed: Jul. 9, 2025

Related U.S. Application Data
(63) Continuation of application No. PCT/CN2024/071997, filed on Jan. 12, 2024.

(30) Foreign Application Priority Data
Jan. 12, 2023 (WO) PCT/CN2023/071969

(51) Int. Cl. C07K 14/54; A61K 35/14; A61K 38/00; A61K 40/31; A61K 40/32; C07K 14/725; C12N 5/078; C12N 5/10; C12N 15/62

(52) U.S. Cl. CPC C07K 14/5428; A61K 35/14; A61K 38/00; A61K 40/31; A61K 40/32; C07K 14/5406; C07K 14/7051; C12N 5/0634; C12N 5/10; C12N 15/625; C07K 2319/02; C12N 2510/00

(57) ABSTRACT

The present disclosure relates to a nucleic acid molecule, a polypeptide, a protein, a cell, or a system comprising a cytokine, optionally a targeting moiety (e.g., a chimeric antigens receptor), and an anchoring structure that can attach to the surface of a cell (e.g., an engineered immune cell), and the methods to prepare the same and to use the same to treat a disease or a condition.

### CROSS REFERENCE TO RELATED APPLICATION

This application is a continuation of International Application No. PCT/CN2024/071997, filed on Jan. 12, 2024, which claims the benefit of international patent application No. PCT/CN2023/071969, filed on Jan. 12, 2023, each of which is incorporated herein by reference in its entirety.

### BACKGROUND

Lymphodepletion is currently a part of the CAR (chimeric antigen receptor)-T cell therapy, which can include administering cyclophosphamide or fludarabine to the patient before infusing CAR-T cells. This step can prevent autologous lymphocytes from attacking the transferred CAR-T cells and prolong the effect of the immune cell therapy. However, the process of removing a large number of autologous lymphocytes makes the medical procedure more complicated, increases the medical burden for the patients as well as the risk of getting an infection after receiving the treatment.

Some researchers have tried co-administering different cytokines to help stimulating the proliferation and improving the survival rate of immune cells in vivo. But the downside of this approach is that it can also stimulate other undesirable lymphocytes to proliferate and increase the risk of cytokine release syndrome (CRS).

There is a need in the art for novel agents that can improve the proliferation and survival of select cells in a targeted fashion.

### SEQUENCE LISTING

This instant application contains a Sequence Listing which has been submitted electronically in XML file format and is hereby incorporated by reference in its entirety. Said XML copy, created on Jul. 1, 2025, is named 60731-701_602_SL.xml and is 478.203 bytes in size.

### SUMMARY

Disclosed herein, in some aspects, is a nucleic acid molecule comprising a nucleic acid sequence that encodes a polypeptide comprising a signal peptide, a cytokine peptide, and a non-peptide anchor attachment signal, wherein the cytokine peptide comprises at least a portion of IL-2, IL-4, IL-7, IL-9, IL-10, IL-18, IL-23, IL-27, IL-36γ, IL-23p19, or IL-1α, or a variant thereof.

Disclosed herein, in some aspects, is an immune cell comprising an exogenous nucleic acid sequence encoding a polypeptide comprising a cytokine peptide and a non-peptide anchor attachment signal, wherein the cytokine peptide comprises at least a portion of IL-2, IL-4, IL-7, IL-9, IL-10, IL-18, IL-23, IL-27, IL-36γ, IL-23p19, or IL-1α, or a variant thereof.

Disclosed herein, in some aspects, is an immune cell comprising an exogenous nucleic acid sequence encoding a polypeptide comprising a cytokine peptide, and a non-peptide anchor attachment signal, wherein the cytokine peptide is at least a portion of IL-12p40 or at least a portion of IL-12p35 and the exogenous nucleic acid sequence does not encode both IL-12p40 and IL-12p35, and wherein the immune cell does not comprise a stimulus response element (SRE) derived from PDE5. In some cases, the immune cell does not comprise a stimulus response element (SRE) and wherein the cytokine peptide is IL-12p40.

Disclosed herein, in some aspects, is an immune cell comprising a protein comprising a cytokine peptide and a non-peptide anchor, wherein the cytokine peptide comprises at least a portion of IL-2, IL-4, IL-7, IL-9, IL-10, IL-18, IL-23, IL-27, IL-36γ, IL-23p19, or IL-1α, or a variant thereof. In some cases, the protein is processed from a polypeptide comprising the cytokine peptide and a non-peptide anchor attachment signal, and wherein the non-peptide anchor attachment is replaced by the non-peptide anchor during protein processing. In some cases, the polypeptide is encoded by an exogenous nucleic acid sequence. In some cases, the polypeptide or the protein comprises a signal peptide. In some cases, the signal peptide, the cytokine peptide, and the non-peptide anchor attachment signal are operably linked in a direction from a N-terminal of the polypeptide to a C-terminal of the polypeptide. In some cases, the non-peptide anchor attachment signal comprises a glycolipid-attachment signal.

Disclosed herein, in some aspects, is a nucleic acid molecule comprising a nucleic acid sequence that encodes a polypeptide comprising a cytokine peptide and a peptide anchor, wherein the cytokine peptide comprises at least a portion of IL-2, IL-4, IL-7, IL-9, IL-10, IL-18, IL-23, IL-27, IL-36γ, IL-23p19, or IL-1α, or a variant thereof.

Disclosed herein, in some aspects, is a cell comprising a polypeptide comprising a cytokine peptide and a peptide anchor, wherein the cytokine peptide comprises at least a portion of IL-2, IL-4, IL-7, IL-9, IL-10, IL-18, IL-23, IL-27, IL-36γ, IL-23p19, or IL-1α, or a variant thereof.

In any one of the foregoing or related aspects, the polypeptide comprises a signal peptide. In some cases, the signal peptide, the cytokine peptide, and the peptide anchor are operably linked in a direction from a N-terminal of the polypeptide to a C-terminal of the polypeptide. In some cases, the peptide anchor comprises a transmembrane peptide sequence. In some cases, the signal peptide comprises CD4 signal peptide, CD8α signal peptide, CD28 signal peptide, CD33 signal peptide, CD137 (4-1BB) signal peptide, IL-2 signal peptide, IgE signal peptide, IgG1 signal peptide. GM-CSF signal peptide, HLA-A signal peptide, HLA signal peptide, TCR signal peptide, or β2M signal peptide, or a combination thereof. In some cases, the signal peptide comprises an amino acid sequence that is at least 75%, 80%, 85%, 90%, 95%, 98%, 99%, or 100% identical to any of the sequences set forth in SEQ ID NOs: 2-7. In some cases, the signal peptide is encoded by a nucleic acid sequence that is at least 75%, 80%, 85%, 90%, 95%, 98%, 99%, or 100% identical to any of the sequences set forth in SEQ ID NOs: 52-57. In some cases, the signal peptide is a naturally occurring signal peptide of a wild-type cytokine. In some cases, the signal peptide comprises an IL-2 signal peptide, an IL-4 signal peptide, an IL-7 signal peptide, an IL-9 signal peptide, an IL-10 signal peptide, an IL-12p40 signal peptide, an IL-15 signal peptide, an IL-18 signal peptide, an IL-21 signal peptide, an IL-23 signal peptide, an IL-27 signal peptide, an IL-36γ signal peptide, an IL-23p19 signal peptide, or an IL-1a signal peptide, or a functional variant thereof. In some cases, the signal peptide comprises an amino acid sequence that is at least 75%, 80%, 85%, 90%, 95%, 98%, 99%, or 100% identical to the sequence set forth in SEQ ID NO: 113 or 114. In some cases, the signal peptide is encoded by a nucleic acid sequence that is at least 75%, 80%, 85%, 90%, 95%, 98%, or 99%, or 100% identical to the sequence set forth in SEQ ID NO: 58 or 59. In some cases, the cytokine peptide comprises at least a portion of IL-2 or a variant thereof. In some cases, the cytokine peptide comprises at least a portion of IL-4 or a variant thereof. In some cases, the cytokine peptide comprises at least a portion of IL-7 or a variant thereof. In some cases, the cytokine peptide comprises at least a portion of IL-9 or a variant thereof. In some cases, the cytokine peptide comprises at least a portion of IL-10 or a variant thereof. In some cases, the cytokine peptide comprises at least a portion of IL-18 or a variant thereof. In some cases, the cytokine peptide comprises at least a portion of IL-23 or a variant thereof. In some cases, the cytokine peptide comprises at least a portion of IL-27 or a variant thereof. In some cases, the cytokine peptide comprises at least a portion of IL-36γ or a variant thereof. In some cases, the cytokine peptide comprises at least a portion of IL-23p19 or a variant thereof. In some cases, the cytokine peptide comprises at least a portion of IL-1a or a variant thereof. In some cases, the variant of the cytokine peptide comprises an alteration, substitution, deletion, addition, or chemical modification of one or more amino acids, one or more unnatural amino acids, or any combination thereof. In some cases, the cytokine peptide comprises an amino acid sequence that is at least 75%, 80%, 85%, 90%, 95%, 98%, 99%, or 100% identical to any of the sequences set forth in SEQ ID NOs: 100-104, 107, or 109-112. In some cases, the cytokine peptide is encoded by a nucleic acid sequence that is at least 75%, 80%, 85%, 90%, 95%, 98%, 99%, or 100% identical to any of the sequences set forth in SEQ ID NOs: 150-154, 157, or 159-164.

In any one of the foregoing or related aspects, the polypeptide or protein further comprises a peptide linker. In some cases, the peptide linker connects the cytokine peptide to the peptide anchor. In some cases, the peptide linker connects the cytokine peptide to the non-peptide anchor attachment signal. In some cases, the peptide linker connects the peptide anchor to the cleavable linker. In some cases, the peptide linker connects the non-peptide anchor attachment signal to the cleavable linker.

In any one of the foregoing or related aspects, the cytotoxicity of said immune cell is increased relative to a comparable immune cell that does not comprise the exogenous nucleic acid sequence encoding the polypeptide comprising the cytokine peptide and the non-peptide anchor attachment signal; does not comprise the nucleic acid sequence encoding the polypeptide comprising the signal peptide, the cytokine peptide and the non-peptide anchor attachment signal; does not comprise the nucleic acid sequence encoding the polypeptide comprising the cytokine peptide and the peptide anchor; or does not comprise protein comprising the cytokine peptide and (i) the non-peptide anchor or (ii) the peptide anchor. In some cases, said increase in cytotoxicity of said immune cell is measured by an in vitro cytotoxicity assay described in Examples A-D. In some cases, said increase in cytotoxicity of said immune cell is measured in vitro or in vivo. In some cases, said increase in cytotoxicity of said immune cell is at least about 5%, 10%, 20%, 30%, 40%, 50% or more.

In any one of the foregoing or related aspects, the population of said immune cells proliferates for a longer period of time relative to a population of comparable immune cells that does not comprise the exogenous nucleic acid sequence encoding the polypeptide comprising the cytokine peptide and the non-peptide anchor attachment signal; does not comprise the nucleic acid sequence encoding the polypeptide comprising the signal peptide, the cytokine peptide and the non-peptide anchor attachment signal; does not comprise the nucleic acid sequence encoding the polypeptide comprising the cytokine peptide and the peptide anchor; or does not comprise protein comprising the cytokine peptide and (i) the non-peptide anchor or (ii) the peptide anchor. In some cases, proliferation of said population of said immune cells is measured by an in vitro immune cell proliferation assay described in Examples A-E. In some cases, proliferation of said population of said immune cells is measured in vitro or in vivo. In some cases, proliferation of said population of said immune cells lasts for a period of time at least about 5%, 10%, 20%, 30%, 40%, 50%, 100%, 2 times, 3 times, 5 times, 10 times or longer than said population of said comparable immune cells.

Disclosed herein, in some aspects, is a nucleic acid molecule comprising a first nucleic acid sequence and a second nucleic acid sequence, wherein: the first nucleic acid sequence encodes a first polypeptide comprising a first cytokine peptide and (i) a first non-peptide anchor attachment signal or (ii) a first peptide anchor; the second nucleic acid sequence encodes a second polypeptide comprising a second cytokine peptide and (i) a second non-peptide anchor attachment signal or (ii) a second peptide anchor; and wherein the first cytokine peptide is a proinflammatory cytokine peptide, and the second cytokine peptide is an anti-inflammatory cytokine peptide.

Disclosed herein, in some aspects, is a system comprising a first nucleic acid sequence and a different second nucleic acid sequence, wherein: the first nucleic acid sequence encodes a first polypeptide comprising a first cytokine peptide and (i) a first non-peptide anchor attachment signal or (ii) a first peptide anchor; the second nucleic acid sequence encodes a second polypeptide comprising a second cytokine peptide and (i) a second non-peptide anchor attachment signal or (ii) a second peptide anchor; and wherein the first cytokine peptide is a proinflammatory cytokine peptide, and the second cytokine peptide is an anti-inflammatory cytokine peptide.

Disclosed herein, in some aspects, is a cell comprising a first protein and a second protein, wherein the first protein comprises a first cytokine peptide and (i) a first non-peptide anchor or (ii) a first peptide anchor; wherein the second protein comprises a second cytokine peptide and (i) a second non-peptide anchor or (ii) a second peptide anchor; and wherein the first cytokine peptide is a proinflammatory cytokine peptide, and the second cytokine peptide is an anti-inflammatory cytokine peptide. In some cases, the first protein is processed from a first polypeptide comprising the first cytokine peptide and (i) a first non-peptide anchor attachment signal or (ii) the first peptide anchor, wherein the first non-peptide anchor attachment signal is replaced by the first non-peptide anchor during protein processing.

In any one of the foregoing or related aspects, the anti-inflammatory cytokine peptide comprises at least a portion of IL-4, IL-10, or IL-27, or a variant thereof, and wherein the proinflammatory cytokine peptide comprises at least a portion of IL-2, IL-7, IL-9, IL-12, IL-12p40, IL-15, IL-18, IL-21, IL-23, IL-36γ, IL-23p19, or IL-1α, or a variant thereof. In some cases, the anti-inflammatory cytokine peptide comprises at least a portion of IL-4 or a variant thereof, and wherein the pro-inflammatory cytokine peptide comprises at least a portion of IL-2, IL-7, IL-9, IL-12, IL-12p40, IL-15, IL-18, IL-21, IL-23, IL-27, IL-36γ, IL-23p19, or IL-1a or a variant thereof. In some cases, the anti-inflammatory cytokine peptide comprises an amino acid sequence that is at least 75%, 80%, 85%, 90%, 95%, 98%, 99%, or 100% identical to the sequence set forth in SEQ ID NO: 101, 104, or 109, and wherein the proinflammatory cytokine peptide comprises an amino acid sequence that is at least 75%, 80%, 85%, 90%, 95%, 98%, 99%, or 100% identical to any one of the sequences set forth in SEQ ID NOs: 100, 102, 103, 105-108, or 110-112. In some cases, the anti-inflammatory cytokine peptide is encoded by a nucleic acid sequence that is at least 75%, 80%, 85%, 90%, 95%, 98%, 99%, or 100% identical to the sequence set forth in SEQ ID NO: 151, 163, 154, 164, or 159, and wherein the proinflammatory cytokine peptide is encoded by a nucleic acid sequence that is at least 75%, 80%, 85%, 90%, 95%, 98%, 99%, or 100% identical to any one of the sequences set forth in SEQ ID NOs: 150, 152, 153, 155-158, or 160-162. In some cases, the anti-inflammatory cytokine peptide comprises an amino acid sequence that is at least 75%, 80%, 85%, 90%, 95%, 98%, 99%, or 100% identical to the sequence set forth in SEQ ID NO: 101, and wherein the pro-inflammatory cytokine peptide comprises an amino acid sequence that is at least 75%, 80%, 85%, 90%, 95%, 98%, 99%, or 100% identical to any one of the sequences set forth in SEQ ID NOs: 100, 102, 103, 105-108, or 110-112. In some cases, the anti-inflammatory cytokine peptide is encoded by a nucleic acid sequence that is at least 75%, 80%, 85%, 90%, 95%, 98%, 99%, or 100% identical to the sequence set forth in SEQ ID NO: 151 or 163, and wherein the proinflammatory cytokine peptide is encoded by a nucleic acid sequence that is at least 75%, 80%, 85%, 90%, 95%, 98%, 99%, or 100% identical to any one of the sequences set forth in SEQ ID NOs: 150, 152, 153, 155-158, or 160-162.

Disclosed herein, in some aspects, is a nucleic acid molecule comprising a first nucleic acid sequence and a second nucleic acid sequence, wherein: the first nucleic acid sequence encodes a first polypeptide comprising a first cytokine peptide and (i) a first non-peptide anchor attachment signal or (ii) a first peptide anchor; the second nucleic acid sequence encodes a second polypeptide comprising a second cytokine peptide and (i) a second non-peptide anchor attachment signal or (ii) a second peptide anchor; and wherein each of the first cytokine peptide and the second cytokine peptide independently comprises at least a portion of IL-2, IL-4, IL-7, IL-9, IL-10, IL-12, IL-12p40, IL-15, IL-18, IL-23, IL-27, IL-36γ, IL-23p19, or IL-1α or a variant thereof. In some cases, the first cytokine peptide comprises an amino acid sequence that is at least 75%, 80%, 85%, 90%, 95%, 98%, 99%, or 100% identical to any of the sequences set forth in SEQ ID NOs: 100-107, or 109-112. In some cases, the first cytokine peptide is encoded by a nucleic acid sequence that is at least 75%, 80%, 85%, 90%, 95%, 98%, 99%, or 100% identical to any of the sequences set forth in SEQ ID NOs: 150-157 or 159-164. In some cases, the second cytokine peptide comprises an amino acid sequence that is at least 75%, 80%, 85%, 90%, 95%, 98%, 99%, or 100% identical to any of the sequences set forth in SEQ ID NOs: 100-107, or 109-112. In some cases, the second cytokine peptide is encoded by a nucleic acid sequence that is at least 75%, 80%, 85%, 90%, 95%, 98%, 99%, or 100% identical to any of the sequences set forth in SEQ ID NOs: 150-157 or 159-164.

Disclosed herein, in some aspects, is a cell comprising a first protein and a second protein, wherein the first protein comprises a first cytokine peptide and (i) a first non-peptide anchor or (ii) a first peptide anchor; wherein the second protein comprises a second cytokine peptide and (i) a second non-peptide anchor or (ii) a second peptide anchor; and wherein each of the first cytokine peptide and the second cytokine peptide independently comprises at least a portion of IL-2, IL-4, IL-7, IL-9, IL-10, IL-12, IL-12p40, IL-15, IL-18, IL-23, IL-27, IL-36γ, IL-23p19, or IL-1α or a variant thereof. In some cases, the first cytokine peptide comprises an amino acid sequence that is at least 75%, 80%, 85%, 90%, 95%, 98%, 99%, or 100% identical to any of the sequences set forth in SEQ ID NOs: 100-107, or 109-112. In some cases, the first cytokine peptide is encoded by a nucleic acid sequence that is at least 75%, 80%, 85%, 90%, 95%, 98%, 99%, or 100% identical to any of the sequences set forth in SEQ ID NOs: 150-157 or 159-164. In some cases, the second cytokine peptide comprises an amino acid sequence that is at least 75%, 80%, 85%, 90%, 95%, 98%, 99%, or 100% identical to any of the sequences set forth in SEQ ID NOs: 100-107, or 109-112. In some cases, the second cytokine peptide is encoded by a nucleic acid sequence that is at least 75%, 80%, 85%, 90%, 95%, 98%, 99%, or 100% identical to any of the sequences set forth in SEQ ID NOs: 150-157 or 159-164.

Disclosed herein, in some aspects, is a system comprising a first nucleic acid sequence and a different second nucleic acid sequence, wherein: the first nucleic acid sequence encodes a first polypeptide comprising a first cytokine peptide and (i) a first non-peptide anchor attachment signal or (ii) a first peptide anchor; the second nucleic acid sequence encodes a second polypeptide comprising a second cytokine peptide and (i) a second non-peptide anchor attachment signal or (ii) a second peptide anchor; and wherein each of the first cytokine peptide and the second cytokine peptide independently comprises at least a portion of IL-2, IL-4, IL-7, IL-9, IL-10, IL-12, IL-12p40, IL-15, IL-18, IL-23, IL-27, IL-36γ, IL-23p19, or IL-1α or a variant thereof. In some cases, the first cytokine peptide comprises an amino acid sequence that is at least 75%, 80%, 85%, 90%, 95%, 98%, 99%, or 100% identical to any of the sequences set forth in SEQ ID NOs: 100-107, or 109-112. In some cases, the first cytokine peptide is encoded by a nucleic acid sequence that is at least 75%, 80%, 85%, 90%, 95%, 98%, 99%, or 100% identical to any of the sequences set forth in SEQ ID NOs: 150-157 or 159-164. In some cases, the second cytokine peptide comprises an amino acid sequence that is at least 75%, 80%, 85%, 90%, 95%, 98%, 99%, or 100% identical to any of the sequences set forth in SEQ ID NOs: 100-107, or 109-112. In some cases, the second cytokine peptide is encoded by a nucleic acid sequence that is at least 75%, 80%, 85%, 90%, 95%, 98%, 99%, or 100% identical to any of the sequences set forth in SEQ ID NOs: 150-157 or 159-164.

In any one of the foregoing or related aspects, the first cytokine peptide comprises at least a portion of IL-4 or a variant thereof, and wherein the second cytokine peptide comprises at least a portion of IL-2, IL-7, IL-9, IL-10, IL-12, IL-12p40, IL-15, IL-18, IL-23, IL-27, IL-36γ, IL-23p19, or IL-1α or a variant thereof. In some cases, the first cytokine peptide comprises an amino acid sequence that is at least 75%, 80%, 85%, 90%, 95%, 98%, 99%, or 100% identical to the sequence set forth in SEQ ID NO: 101, and wherein the second cytokine peptide comprises an amino acid sequence that is at least 75%, 80%, 85%, 90%, 95%, 98%, 99%, or 100% identical to any one of the sequences set forth in SEQ ID NOs: 100, 102-107, or 109-112. In some cases, the first cytokine peptide is encoded by a nucleic acid sequence that is at least 75%, 80%, 85%, 90%, 95%, 98%, 99%, or 100% identical to the sequence set forth in SEQ ID NO: 151 or 163, and wherein the second cytokine peptide is encoded by a nucleic acid sequence that is at least 75%, 80%, 85%, 90%, 95%, 98%, 99%, or 100% identical to any one of the sequences set forth in SEQ ID NOs: 150, 152-157, 159-162, or 164.

Disclosed herein, in some aspects, is a nucleic acid molecule comprising a first nucleic acid sequence and a second nucleic acid sequence, wherein: the first nucleic acid sequence encodes a first polypeptide comprising a first cytokine peptide and (i) a first non-peptide anchor attachment signal or (ii) a first peptide anchor; the second nucleic acid sequence encodes a second polypeptide comprising a second cytokine peptide and (i) a second non-peptide anchor attachment signal or (ii) a second peptide anchor; and wherein each of the first cytokine peptide and the second cytokine peptide independently comprises at least a portion of IL-2, IL-4, IL-7, IL-9, IL-10, IL-12, IL-12p40, IL-18, IL-21, IL-23, IL-27, IL-36γ, IL-23p19, or IL-1α or a variant thereof. In some cases, the first cytokine peptide comprises an amino acid sequence that is at least 75%, 80%, 85%, 90%, 95%, 98%, 99%, or 100% identical to any of the sequences set forth in SEQ ID NOs: 100-105 or 107-112. In some cases, the first cytokine peptide is encoded by a nucleic acid sequence that is at least 75%, 80%, 85%, 90%, 95%, 98%, 99%, or 100% identical to any of the sequences set forth in SEQ ID NOs: 150-155 or 157-164. In some cases, the second cytokine peptide comprises an amino acid sequence that is at least 75%, 80%, 85%, 90%, 95%, 98%, 99%, or 100% identical to any of the sequences set forth in SEQ ID NOs: 100-105 or 107-112. In some cases, the second cytokine peptide is encoded by a nucleic acid sequence that is at least 75%, 80%, 85%, 90%, 95%, 98%, 99%, or 100% identical to any of the sequences set forth in SEQ ID NOs: 150-155 or 157-164.

Disclosed herein, in some aspects, is a system comprising a first nucleic acid sequence and a different second nucleic acid sequence, wherein: the first nucleic acid sequence encodes a first polypeptide comprising a first cytokine peptide and (i) a first non-peptide anchor attachment signal or (ii) a first peptide anchor; the second nucleic acid sequence encodes a second polypeptide comprising a second cytokine peptide and (i) a second non-peptide anchor attachment signal or (ii) a second peptide anchor; and wherein each of the first cytokine peptide and the second cytokine peptide independently comprises at least a portion of IL-2, IL-4, IL-7, IL-9, IL-10, IL-12, IL-12p40, IL-18, IL-21, IL-23, IL-27, IL-36γ, IL-23p19, or IL-1α or a variant thereof. In some cases, the first cytokine peptide comprises an amino acid sequence that is at least 75%, 80%, 85%, 90%, 95%, 98%, 99%, or 100% identical to any of the sequences set forth in SEQ ID NOs: 100-105 or 107-112. In some cases, the first cytokine peptide is encoded by a nucleic acid sequence that is at least 75%, 80%, 85%, 90%, 95%, 98%, 99%, or 100% identical to any of the sequences set forth in SEQ ID NOs: 150-155 or 157-164. In some cases, the second cytokine peptide comprises an amino acid sequence that is at least 75%, 80%, 85%, 90%, 95%, 98%, 99%, or 100% identical to any of the sequences set forth in SEQ ID NOs: 100-105 or 107-112. In some cases, the second cytokine peptide is encoded by a nucleic acid sequence that is at least 75%, 80%, 85%, 90%, 95%, 98%, 99%, or 100% identical to any of the sequences set forth in SEQ ID NOs: 150-155 or 157-164.

Disclosed herein, in some aspects, is a cell comprising a first protein and a second protein, wherein the first protein comprises a first cytokine peptide and (i) a first non-peptide anchor or (ii) a first peptide anchor; wherein the second protein comprises a second cytokine peptide and (i) a second non-peptide anchor or (ii) a second peptide anchor; and wherein each of the first cytokine peptide and the second cytokine peptide independently comprises at least a portion of IL-2, IL-4, IL-7, IL-9, IL-10, IL-12, IL-12p40, IL-18, IL-21, IL-23, IL-27, IL-36γ, IL-23p19, or IL-1α or a variant thereof. In some cases, the first cytokine peptide comprises an amino acid sequence that is at least 75%, 80%, 85%, 90%, 95%, 98%, 99%, or 100% identical to any of the sequences set forth in SEQ ID NOs: 100-105 or 107-112. In some cases, the first cytokine peptide is encoded by a nucleic acid sequence that is at least 75%, 80%, 85%, 90%, 95%, 98%, 99%, or 100% identical to any of the sequences set forth in SEQ ID NOs: 150-155 or 157-164. In some cases, the second cytokine peptide comprises an amino acid sequence that is at least 75%, 80%, 85%, 90%, 95%, 98%, 99%, or 100% identical to any of the sequences set forth in SEQ ID NOs: 100-105 or 107-112. In some cases, the second cytokine peptide is encoded by a nucleic acid sequence that is at least 75%, 80%, 85%, 90%, 95%, 98%, 99%, or 100% identical to any of the sequences set forth in SEQ ID NOs: 150-155 or 157-164.

In any one of the foregoing or related aspects, the first cytokine peptide comprises at least a portion of IL-4 or a variant thereof, and wherein the second cytokine peptide comprises at least a portion of IL-2, IL-7, IL-9, IL-10, IL-12, IL-12p40, IL-18, IL-21, IL-23, IL-27, IL-36γ, IL-23p19, or IL-1α or a variant thereof. In some cases, the first cytokine peptide comprises an amino acid sequence that is at least 75%, 80%, 85%, 90%, 95%, 98%, 99%, or 100% identical to the sequence set forth in SEQ ID NO: 101, and wherein the second cytokine peptide comprises an amino acid sequence that is at least 75%, 80%, 85%, 90%, 95%, 98%, 99%, or 100% identical to any one of the sequences set forth in SEQ ID NOs: 100, 102-105, or 107-112. In some cases, the first cytokine peptide is encoded by a nucleic acid sequence that is at least 75%, 80%, 85%, 90%, 95%, 98%, 99%, or 100% identical to the sequence set forth in SEQ ID NO: 151 or 163, and wherein the second cytokine peptide is encoded by a nucleic acid sequence that is at least 75%, 80%, 85%, 90%, 95%, 98%, 99%, or 100% identical to any one of the sequences set forth in SEQ ID NOs: 150, 152-155, 157-162, or 164.

In any one of the foregoing or related aspects, the cytotoxicity of said immune cell is increased relative to a comparable immune cell that does not comprise both of: (1) the first nucleic acid sequence encodes the first polypeptide comprising the first cytokine peptide and (i) the first non-peptide anchor attachment signal or (ii) the first peptide anchor; and (2) the second nucleic acid sequence encodes the second polypeptide comprising the second cytokine peptide and (i) the second non-peptide anchor attachment signal or (ii) the second peptide anchor; or does not comprise both of: (1) the first protein comprising the first cytokine peptide and (i) the first non-peptide anchor or (ii) the first peptide anchor; and (2) the second protein comprising the second cytokine peptide and (i) the second non-peptide anchor or (ii) the second peptide anchor. In some cases, said increase in cytotoxicity of said immune cell is measured by an in vitro cytotoxicity assay described in Examples A-D. In some cases, said increase in cytotoxicity of said immune cell is measured in vitro or in vivo. In some cases, said increase in cytotoxicity of said immune cell is at least about 5%, 10%, 20%, 30%, 40%, 50% or more.

In any one of the foregoing or related aspects, the population of said immune cells proliferates for a longer period of time relative to a population of comparable immune cells that does not comprise the first nucleic acid sequence encodes the first polypeptide comprising the first cytokine peptide and (i) the first non-peptide anchor attachment signal or (ii) the first peptide anchor, and/or does not comprise the second nucleic acid sequence encodes the second polypeptide comprising the second cytokine peptide and (i) the second non-peptide anchor attachment signal or (ii) the second peptide anchor; or does not comprise the first protein comprising the first cytokine peptide and (i) the first non-peptide anchor or (ii) the first peptide anchor, and/or does not comprise the second protein comprising the second cytokine peptide and (i) the second non-peptide anchor or (ii) the second peptide anchor. In some cases, proliferation of said population of said immune cells is measured by an in vitro immune cell proliferation assay described in Examples A-E. In some cases, proliferation of said population of said immune cells is measured in vitro or in vivo. In some cases, proliferation of said population of said immune cells lasts for a period of time at least about 5%, 10%, 20%, 30%, 40%, 50%, 100%, 2 times, 3 times, 5 times, 10 times or longer than said population of said comparable immune cells.

In any one of the foregoing or related aspects, the variant of the first cytokine peptide and the second cytokine peptide comprises an alteration, substitution, deletion, addition, or chemical modification of one or more amino acids, one or more unnatural amino acids, or any combination thereof.

In any one of the foregoing or related aspects, the first cytokine peptide comprises at least a portion of IL-2 or a variant thereof, and the second cytokine peptide comprises at least a portion of IL-7 or a variant thereof. In some cases, the first cytokine peptide comprises at least a portion of IL-2 or a variant thereof, and the second cytokine peptide comprises at least a portion of IL-12 or a variant thereof. In some cases, the first cytokine peptide comprises at least a portion of IL-2 or a variant thereof, and the second cytokine peptide comprises at least a portion of IL-15 or a variant thereof. In some cases, the first cytokine peptide comprises at least a portion of IL-2 or a variant thereof, and the second cytokine peptide comprises at least a portion of IL-21 or a variant thereof. In some cases, the first cytokine peptide comprises at least a portion of IL-2 or a variant thereof, and the second cytokine peptide comprises at least a portion of IL-12p40 or a variant thereof. In some cases, the first cytokine peptide comprises at least a portion of IL-2 or a variant thereof, and the second cytokine peptide comprises at least a portion of IL-23p19 or a variant thereof. In some cases, the first cytokine peptide comprises at least a portion of IL-7 or a variant thereof, and the second cytokine peptide comprises at least a portion of IL-12 or a variant thereof. In some cases, the first cytokine peptide comprises at least a portion of IL-7 or a variant thereof, and the second cytokine peptide comprises at least a portion of IL-15 or a variant thereof. In some cases, the first cytokine peptide comprises at least a portion of IL-7 or a variant thereof, and the second cytokine peptide comprises at least a portion of IL-21 or a variant thereof. In some cases, the first cytokine peptide comprises at least a portion of IL-7 or a variant thereof, and the second cytokine peptide comprises at least a portion of IL-12p40 or a variant thereof. In some cases, the first cytokine peptide comprises at least a portion of IL-7 or a variant thereof, and the second cytokine peptide comprises at least a portion of IL-23p19 or a variant thereof. In some cases, the first cytokine peptide comprises at least a portion of IL-12 or a variant thereof, and the second cytokine peptide comprises at least a portion of IL-15 or a variant thereof. In some cases, the first cytokine peptide comprises at least a portion of IL-12 or a variant thereof, and the second cytokine peptide comprises at least a portion of IL-21 or a variant thereof. In some cases, the first cytokine peptide comprises at least a portion of IL-12 or a variant thereof, and the second cytokine peptide comprises at least a portion of IL-12p40 or a variant thereof. In some cases, the first cytokine peptide comprises at least a portion of IL-12 or a variant thereof, and the second cytokine peptide comprises at least a portion of IL-23p19 or a variant thereof. In some cases, the first cytokine peptide comprises at least a portion of IL-15 or a variant thereof, and the second cytokine peptide comprises at least a portion of IL-12p40 or a variant thereof. In some cases, the first cytokine peptide comprises at least a portion of IL-15 or a variant thereof, and the second cytokine peptide comprises at least a portion of IL-23p19 or a variant thereof. In some cases, the first cytokine peptide comprises at least a portion of IL-21 or a variant thereof, and the second cytokine peptide comprises at least a portion of IL-12p40 or a variant thereof. In some cases, the first cytokine peptide comprises at least a portion of IL-21 or a variant thereof, and the second cytokine peptide comprises at least a portion of IL-23p19 or a variant thereof. In some cases, the first cytokine peptide comprises at least a portion of IL-12p40 or a variant thereof, and the second cytokine peptide comprises at least a portion of IL-23p19 or a variant thereof.

Disclosed herein, in some aspects, is a nucleic acid molecule comprising a first nucleic acid sequence, a second nucleic acid sequence, and a third nucleic acid sequence, wherein: the first nucleic acid sequence encodes a first polypeptide comprising a first cytokine peptide and (i) a first non-peptide anchor attachment signal or (ii) a first peptide anchor, the second nucleic acid sequence encodes a second polypeptide comprising a second cytokine peptide and (i) a second non-peptide anchor attachment signal or (ii) a second peptide anchor, the third nucleic acid sequence encodes a third polypeptide comprising a third cytokine peptide and (i) a third non-peptide anchor attachment signal or (ii) a third peptide anchor, wherein each of the first cytokine peptide, the second cytokine peptide, and the third cytokine peptide independently comprises at least a portion of IL-2, IL-4, IL-7, IL-9, IL-10, IL-12, IL-12p40, IL-15, IL-18, IL-21, IL-23, IL-27, IL-36γ, IL-23p19, or IL-1α or a variant thereof.

Disclosed herein, in some aspects, is a system comprising a first nucleic acid sequence, a second nucleic acid sequence, and a third nucleic acid sequence, wherein: the first nucleic acid sequence encodes a first polypeptide comprising a first cytokine peptide and (i) a first non-peptide anchor attachment signal or (ii) a first peptide anchor, the second nucleic acid sequence encodes a second polypeptide comprising a second cytokine peptide and (i) a second non-peptide anchor attachment signal or (ii) a second peptide anchor, the third nucleic acid sequence encodes a third polypeptide comprising a third cytokine peptide and (i) a third non-peptide anchor attachment signal or (ii) a third peptide anchor, wherein each of the first cytokine peptide, the second cytokine peptide, and the third cytokine peptide independently comprises at least a portion of IL-2, IL-4, IL-7, IL-9, IL-10, IL-12, IL-12p40, IL-15, IL-18, IL-21, IL-23, IL-27, IL-36γ, IL-23p19, or IL-1α or a variant thereof, and wherein the first nucleic acid sequence, the second nucleic acid sequence, and the third nucleic acid sequence are different.

Disclosed herein, in some aspects, is a cell comprising a first protein, a second protein, and a third protein, wherein: the first protein comprises a first cytokine peptide and (i) a first non-peptide anchor or (ii) a first peptide anchor, the second protein comprises a second cytokine peptide and (i) a second non-peptide anchor or (ii) a second peptide anchor, the third protein comprises a third cytokine peptide and (i) a third non-peptide anchor or (ii) a third peptide anchor, wherein each of the first cytokine peptide, the second cytokine peptide, and the third cytokine peptide independently comprises at least a portion of IL-2, IL-4, IL-7, IL-9, IL-10, IL-12, IL-12p40, IL-15, IL-18, IL-21, IL-23, IL-27, IL-36γ, IL-23p19, or IL-1α or a variant thereof.

In any one of the foregoing or related aspects, the cytotoxicity of said immune cell is increased relative to a comparable immune cell that does not comprise all of: (1) the first nucleic acid sequence encodes the first polypeptide comprising the first cytokine peptide and (i) a first non-peptide anchor attachment signal or (ii) the first peptide anchor; (2) the second nucleic acid sequence encodes the second polypeptide comprising the second cytokine peptide and (i) the second non-peptide anchor attachment signal or (ii) the second peptide anchor; and (3) the third nucleic acid sequence encodes the third polypeptide comprising the third cytokine peptide and (i) the third non-peptide anchor attachment signal or (ii) the third peptide anchor; or does not comprise all of: (1) the first protein comprising the first cytokine peptide and (i) the first non-peptide anchor or (ii) the first peptide anchor; (2) the second protein comprising the second cytokine peptide and (i) the second non-peptide anchor or (ii) the second peptide anchor; and (3) the third protein comprising the third cytokine peptide and (i) the third non-peptide anchor or (ii) the third peptide anchor. In some cases, said increase in cytotoxicity of said immune cell is measured by an in vitro cytotoxicity assay described in Examples A-D. In some cases, said increase in cytotoxicity of said immune cell is measured in vitro or in vivo. In some cases, said increase in cytotoxicity of said immune cell is at least about 5%, 10%, 20%, 30%, 40%, 50% or more.

In any one of the foregoing or related aspects, the population of said immune cells proliferates for a longer period of time relative to a population of comparable immune cells that does not comprise all of: (1) the first nucleic acid sequence encodes the first polypeptide comprising the first cytokine peptide and (i) a first non-peptide anchor attachment signal or (ii) the first peptide anchor; (2) the second nucleic acid sequence encodes the second polypeptide comprising the second cytokine peptide and (i) the second non-peptide anchor attachment signal or (ii) the second peptide anchor; and (3) the third nucleic acid sequence encodes the third polypeptide comprising the third cytokine peptide and (i) the third non-peptide anchor attachment signal or (ii) the third peptide anchor; or does not comprise all of: (1) the first protein comprising the first cytokine peptide and (i) the first non-peptide anchor or (ii) the first peptide anchor; (2) the second protein comprising the second cytokine peptide and (i) the second non-peptide anchor or (ii) the second peptide anchor; and (3) the third protein comprising the third cytokine peptide and (i) the third non-peptide anchor or (ii) the third peptide anchor. In some cases, proliferation of said population of said immune cells is measured by an in vitro immune cell proliferation assay described in Examples A-E. In some cases, proliferation of said population of said immune cells is measured in vitro or in vivo. In some cases, proliferation of said population of said immune cells lasts for a period of time at least about 5%, 10%, 20%, 30%, 40%, 50%, 100%, 2 times, 3 times, 5 times, 10 times or longer than said population of said comparable immune cells.

In any one of the foregoing or related aspects, the first protein is processed from a first polypeptide comprising the first cytokine peptide and (i) a first non-peptide anchor attachment signal or (ii) the first peptide anchor, wherein the first non-peptide anchor attachment signal is replaced by the first non-peptide anchor during protein processing. In some cases, the first polypeptide is encoded by a first nucleic acid sequence. In any one of the foregoing or related aspects, the second protein is processed from a second polypeptide comprising the second cytokine peptide and (i) a second non-peptide anchor attachment signal or (ii) the second peptide anchor, wherein the second non-peptide anchor attachment signal is replaced by the second non-peptide anchor during protein processing. In some cases, the second polypeptide is encoded by a second nucleic acid sequence. In some cases, the second nucleic acid sequence and the first nucleic acid sequence are under control of a same promoter. In some cases, the second nucleic acid sequence and the first nucleic acid sequence are under control of two different promoters. In some cases, the first nucleic acid sequence and the second nucleic acid sequence are operably linked in a 5′ to 3′ direction. In some cases, the second nucleic acid sequence and the first nucleic acid sequence are operably linked in a 5′ to 3′ direction. In some cases, the second nucleic acid sequence and the first nucleic acid sequence are linked via a nucleic acid sequence encoding a cleavable linker. In some cases, the first protein or the first polypeptide comprises a first signal peptide. In some cases, the first signal peptide, the first cytokine peptide, and (i) the first non-peptide anchor attachment signal or (ii) the first peptide anchor are operably linked in a direction from a N-terminal of the polypeptide to a C-terminal of the polypeptide. In some cases, the second protein or the second polypeptide comprises a second signal peptide. In some cases, the second signal peptide, the second cytokine peptide, and (i) the second non-peptide anchor attachment signal or (ii) the second peptide anchor are operably linked in a direction from a N-terminal of the polypeptide to a C-terminal of the polypeptide. In some cases, the first non-peptide anchor attachment signal comprises a glycolipid-attachment signal. In some cases, the second non-peptide anchor attachment signal comprises a glycolipid-attachment signal.

In any one of the foregoing or related aspects, the third protein is processed from a third polypeptide comprising the third cytokine peptide and a third non-peptide anchor attachment signal or the third peptide anchor, wherein the third non-peptide anchor attachment signal is replaced by the third non-peptide anchor during protein processing. In some cases, the third polypeptide is encoded by a third nucleic acid sequence. In some cases, the second nucleic acid sequence and the third nucleic acid sequence are under control of a same promoter. In some cases, the second nucleic acid sequence and the third nucleic acid sequence are under control of two different promoters. In some cases, the first nucleic acid sequence and the third nucleic acid sequence are under control of a same promoter. In some cases, the first nucleic acid sequence and the third nucleic acid sequence are under control of two different promoters. In some cases, the first nucleic acid sequence, the second nucleic acid sequence, and the third nucleic acid sequence, are operably linked in a 5′ to 3′ direction. In some cases, the first nucleic acid sequence, the third nucleic acid sequence, and the second nucleic acid sequence are operably linked in a 5′ to 3′ direction. In some cases, the second nucleic acid sequence, the first nucleic acid sequence, and the third nucleic acid sequence, are operably linked in a 5′ to 3′ direction. In some cases, the second nucleic acid sequence, the third nucleic acid sequence, and the first nucleic acid sequence are operably linked in a 5′ to 3′ direction. In some cases, the third nucleic acid sequence, the second nucleic acid sequence, and the first nucleic acid sequence are operably linked in a 5′ to 3′ direction. In some cases, the third nucleic acid sequence, the first nucleic acid sequence, and the second nucleic acid sequence are operably linked in a 5′ to 3′ direction. In some cases, the first nucleic acid sequence and the second nucleic acid sequence are linked via a nucleic acid sequence encoding a cleavable linker. In some cases, the second nucleic acid sequence and the third nucleic acid sequence are linked via a nucleic acid sequence encoding a cleavable linker. In some cases, the first nucleic acid sequence and the third nucleic acid sequence are linked via a nucleic acid sequence encoding a cleavable linker.

In any one of the foregoing or related aspects, the third protein or the third polypeptide comprises a third signal peptide. In some cases, the third signal peptide, the third cytokine peptide, and (i) the third non-peptide anchor attachment signal or (ii) the third peptide anchor are operably linked in a direction from a N-terminal of the polypeptide to a C-terminal of the polypeptide. In some cases, the third non-peptide anchor attachment signal comprises a glycolipid-attachment signal.

In any one of the foregoing or related aspects, each of the first signal peptide, the second signal peptide, and the third signal peptide independently comprises CD4 signal peptide, CD8α signal peptide. CD28 signal peptide, CD33 signal peptide, CD137 (4-1BB) signal peptide, IL-2 signal peptide, IgE signal peptide, IgG1 signal peptide. GM-CSF signal peptide, HLA-A signal peptide, HLA signal peptide. TCR signal peptide, or β2M signal peptide, or a combination thereof. In some cases, each of the first signal peptide, the second signal peptide, and the third signal peptide independently comprises an amino acid sequence that is at least 75%, 80%, 85%, 90%, 95%, 98%, 99%, or 100% identical to any of the sequences set forth in SEQ ID NOs: 2-7. In some cases, each of the first signal peptide, the second signal peptide, and the third signal peptide is independently encoded by a nucleic acid sequence that is at least 75%, 80%, 85%, 90%, 95%, 98%, 99%, or 100% identical to any of the sequences set forth in SEQ ID NOs: 52-57.

In any one of the forgoing or related aspect, the first cytokine peptide comprises an amino acid sequence that is at least 75%, 80%, 85%, 90%, 95%, 98%, 99%, or 100% identical to any of the sequences set forth in SEQ ID NOs: 100-112. In some cases, the first cytokine peptide is encoded by a nucleic acid sequence that is at least 75%, 80%, 85%, 90%, 95%, 98%, 99%, or 100% identical to any of the sequences set forth in SEQ ID NOs: 150-164. In some cases, the first cytokine peptide comprises at least a portion of IL-2 or a variant thereof. In some cases, the first cytokine peptide comprises at least a portion of IL-4 or a variant thereof. In some cases, the first cytokine peptide comprises at least a portion of IL-7 or a variant thereof. In some cases, the first cytokine peptide comprises at least a portion of IL-9 or a variant thereof. In some cases, the first cytokine peptide comprises at least a portion of IL-10 or a variant thereof. In some cases, the first cytokine peptide comprises at least a portion of IL-12 or a variant thereof. In some cases, the first cytokine peptide comprises at least a portion of IL-12p40 or a variant thereof. In some cases, the first cytokine peptide comprises at least a portion of IL-18 or a variant thereof. In some cases, the first cytokine peptide comprises at least a portion of IL-23 or a variant thereof. In some cases, the first cytokine peptide comprises at least a portion of IL-27 or a variant thereof. In some cases, the first cytokine peptide comprises at least a portion of IL-36γ or a variant thereof. In some cases, the first cytokine peptide comprises at least a portion of IL-23p19 or a variant thereof. In some cases, the first cytokine peptide comprises at least a portion of IL-1α or a variant thereof. In some cases, the first cytokine peptide comprises at least a portion of IL-15 or a variant thereof. In some cases, the first cytokine peptide comprises at least a portion of IL-21 or a variant thereof.

In any one of the forgoing or related aspect, the second cytokine peptide comprises an amino acid sequence that is at least 75%, 80%, 85%, 90%, 95%, 98%, 99%, or 100% identical to any of the sequences set forth in SEQ ID NOs: 100-112. In some cases, the second cytokine peptide is encoded by a nucleic acid sequence that is at least 75%, 80%, 85%, 90%, 95%, 98%, 99%, or 100% identical to any of the sequences set forth in SEQ ID NOs: 150-164. In some cases, the second cytokine peptide comprises at least a portion of IL-2 or a variant thereof. In some cases, the second cytokine peptide comprises at least a portion of IL-4 or a variant thereof. In some cases, the second cytokine peptide comprises at least a portion of IL-7 or a variant thereof. In some cases, the second cytokine peptide comprises at least a portion of IL-9 or a variant thereof. In some cases, the second cytokine peptide comprises at least a portion of IL-10 or a variant thereof. In some cases, the second cytokine peptide comprises at least a portion of IL-12 or a variant thereof. In some cases, the second cytokine peptide comprises at least a portion of IL-12p40 or a variant thereof. In some cases, the second cytokine peptide comprises at least a portion of IL-18 or a variant thereof. In some cases, the second cytokine peptide comprises at least a portion of IL-23 or a variant thereof. In some cases, the second cytokine peptide comprises at least a portion of IL-27 or a variant thereof. In some cases, the second cytokine peptide comprises at least a portion of IL-36γ or a variant thereof. In some cases, the second cytokine peptide comprises at least a portion of IL-23p19 or a variant thereof. In some cases, the second cytokine peptide comprises at least a portion of IL-1α or a variant thereof. In some cases, the second cytokine peptide comprises at least a portion of IL-15 or a variant thereof. In some cases, the second cytokine peptide comprises at least a portion of IL-21 or a variant thereof.

In any one of the forgoing or related aspect, the third cytokine peptide comprises an amino acid sequence that is at least 75%, 80%, 85%, 90%, 95%, 98%, 99%, or 100% identical to any of the sequences set forth in SEQ ID NOs: 100-112. In some cases, the third cytokine peptide is encoded by a nucleic acid sequence that is at least 75%, 80%, 85%, 90%, 95%, 98%, 99%, or 100% identical to any of the sequences set forth in SEQ ID NOs: 150-164. In some cases, the third cytokine peptide comprises at least a portion of IL-2 or a variant thereof. In some cases, the third cytokine peptide comprises at least a portion of IL-4 or a variant thereof. In some cases, the third cytokine peptide comprises at least a portion of IL-7 or a variant thereof. In some cases, the third cytokine peptide comprises at least a portion of IL-9 or a variant thereof. In some cases, the third cytokine peptide comprises at least a portion of IL-10 or a variant thereof. In some cases, the third cytokine peptide comprises at least a portion of IL-12 or a variant thereof. In some cases, the third cytokine peptide comprises at least a portion of IL-12p40 or a variant thereof. In some cases, the third cytokine peptide comprises at least a portion of IL-18 or a variant thereof. In some cases, the third cytokine peptide comprises at least a portion of IL-23 or a variant thereof. In some cases, the third cytokine peptide comprises at least a portion of IL-27 or a variant thereof. In some cases, the third cytokine peptide comprises at least a portion of IL-36γ or a variant thereof. In some cases, the third cytokine peptide comprises at least a portion of IL-23p19 or a variant thereof. In some cases, the third cytokine peptide comprises at least a portion of IL-1α or a variant thereof. In some cases, the third cytokine peptide comprises at least a portion of IL-15 or a variant thereof. In some cases, the third cytokine peptide comprises at least a portion of IL-21 or a variant thereof. In some cases, the first cytokine peptide comprises at least a portion of IL-2 or a variant thereof, the second cytokine peptide comprises at least a portion of IL-7 or a variant thereof, and the third cytokine peptide comprises at least a portion of IL-12 or a variant thereof. In some cases, the first cytokine peptide comprises at least a portion of IL-2 or a variant thereof, the second cytokine peptide comprises at least a portion of IL-7 or a variant thereof, and the third cytokine peptide comprises at least a portion of IL-15 or a variant thereof. In some cases, the first cytokine peptide comprises at least a portion of IL-2 or a variant thereof, the second cytokine peptide comprises at least a portion of IL-7 or a variant thereof, and the third cytokine peptide comprises at least a portion of IL-21 or a variant thereof. In some cases, the first cytokine peptide comprises at least a portion of IL-2 or a variant thereof, the second cytokine peptide comprises at least a portion of IL-7 or a variant thereof, and the third cytokine peptide comprises at least a portion of IL-12p40 or a variant thereof. In some cases, the first cytokine peptide comprises at least a portion of IL-2 or a variant thereof, the second cytokine peptide comprises at least a portion of IL-7 or a variant thereof, and the third cytokine peptide comprises at least a portion of IL-23p19 or a variant thereof. In some cases, the first cytokine peptide comprises at least a portion of IL-2 or a variant thereof, the second cytokine peptide comprises at least a portion of IL-12 or a variant thereof, and the third cytokine peptide comprises at least a portion of IL-15 or a variant thereof. In some cases, the first cytokine peptide comprises at least a portion of IL-2 or a variant thereof, the second cytokine peptide comprises at least a portion of IL-12 or a variant thereof, and the third cytokine peptide comprises at least a portion of IL-21 or a variant thereof. In some cases, the first cytokine peptide comprises at least a portion of IL-2 or a variant thereof, the second cytokine peptide comprises at least a portion of IL-12 or a variant thereof, and the third cytokine peptide comprises at least a portion of IL-12p40 or a variant thereof. In some cases, the first cytokine peptide comprises at least a portion of IL-2 or a variant thereof, the second cytokine peptide comprises at least a portion of IL-12 or a variant thereof, and the third cytokine peptide comprises at least a portion of IL-23p19 or a variant thereof. In some cases, the first cytokine peptide comprises at least a portion of IL-2 or a variant thereof, the second cytokine peptide comprises at least a portion of IL-15 or a variant thereof, and the third cytokine peptide comprises at least a portion of IL-21 or a variant thereof. In some cases, the first cytokine peptide comprises at least a portion of IL-2 or a variant thereof, the second cytokine peptide comprises at least a portion of IL-15 or a variant thereof, and the third cytokine peptide comprises at least a portion of IL-12p40 or a variant thereof. In some cases, the first cytokine peptide comprises at least a portion of IL-2 or a variant thereof, the second cytokine peptide comprises at least a portion of IL-15 or a variant thereof, and the third cytokine peptide comprises at least a portion of IL-23p19 or a variant thereof. In some cases, the first cytokine peptide comprises at least a portion of IL-2 or a variant thereof, the second cytokine peptide comprises at least a portion of IL-21 or a variant thereof, and the third cytokine peptide comprises at least a portion of IL-12p40 or a variant thereof. In some cases, the first cytokine peptide comprises at least a portion of IL-2 or a variant thereof, the second cytokine peptide comprises at least a portion of IL-21 or a variant thereof, and the third cytokine peptide comprises at least a portion of IL-23p19 or a variant thereof. In some cases, the first cytokine peptide comprises at least a portion of IL-2 or a variant thereof, the second cytokine peptide comprises at least a portion of IL-12p40 or a variant thereof, and the third cytokine peptide comprises at least a portion of IL-23p19 or a variant thereof. In some cases, the first cytokine peptide comprises at least a portion of IL-7 or a variant thereof, the second cytokine peptide comprises at least a portion of IL-12 or a variant thereof, and the third cytokine peptide comprises at least a portion of IL-15 or a variant thereof. In some cases, the first cytokine peptide comprises at least a portion of IL-7 or a variant thereof, the second cytokine peptide comprises at least a portion of IL-12 or a variant thereof, and the third cytokine peptide comprises at least a portion of IL-21 or a variant thereof. In some cases, the first cytokine peptide comprises at least a portion of IL-7 or a variant thereof, the second cytokine peptide comprises at least a portion of IL-12 or a variant thereof, and the third cytokine peptide comprises at least a portion of IL-12p40 or a variant thereof. In some cases, the first cytokine peptide comprises at least a portion of IL-7 or a variant thereof, the second cytokine peptide comprises at least a portion of IL-12 or a variant thereof, and the third cytokine peptide comprises at least a portion of IL-23p19 or a variant thereof. In some cases, the first cytokine peptide comprises at least a portion of IL-7 or a variant thereof, the second cytokine peptide comprises at least a portion of IL-15 or a variant thereof, and the third cytokine peptide comprises at least a portion of IL-21 or a variant thereof. In some cases, the first cytokine peptide comprises at least a portion of IL-7 or a variant thereof, the second cytokine peptide comprises at least a portion of IL-15 or a variant thereof, and the third cytokine peptide comprises at least a portion of IL-12p40 or a variant thereof. In some cases, the first cytokine peptide comprises at least a portion of IL-7 or a variant thereof, the second cytokine peptide comprises at least a portion of IL-15 or a variant thereof, and the third cytokine peptide comprises at least a portion of IL-23p19 or a variant thereof. In some cases, the first cytokine peptide comprises at least a portion of IL-7 or a variant thereof, the second cytokine peptide comprises at least a portion of IL-21 or a variant thereof, and the third cytokine peptide comprises at least a portion of IL-12p40 or a variant thereof. In some cases, the first cytokine peptide comprises at least a portion of IL-7 or a variant thereof, the second cytokine peptide comprises at least a portion of IL-21 or a variant thereof, and the third cytokine peptide comprises at least a portion of IL-23p19 or a variant thereof. In some cases, the first cytokine peptide comprises at least a portion of IL-7 or a variant thereof, the second cytokine peptide comprises at least a portion of IL-12p40 or a variant thereof, and the third cytokine peptide comprises at least a portion of IL-23p19 or a variant thereof. In some cases, the first cytokine peptide comprises at least a portion of IL-12 or a variant thereof, the second cytokine peptide comprises at least a portion of IL-15 or a variant thereof, and the third cytokine peptide comprises at least a portion of IL-21 or a variant thereof. In some cases, the first cytokine peptide comprises at least a portion of IL-12 or a variant thereof, the second cytokine peptide comprises at least a portion of IL-15 or a variant thereof, and the third cytokine peptide comprises at least a portion of IL-12p40 or a variant thereof. In some cases, the first cytokine peptide comprises at least a portion of IL-12 or a variant thereof, the second cytokine peptide comprises at least a portion of IL-15 or a variant thereof, and the third cytokine peptide comprises at least a portion of IL-23p19 or a variant thereof. In some cases, the first cytokine peptide comprises at least a portion of IL-12 or a variant thereof, the second cytokine peptide comprises at least a portion of IL-21 or a variant thereof, and the third cytokine peptide comprises at least a portion of IL-12p40 or a variant thereof. In some cases, the first cytokine peptide comprises at least a portion of IL-12 or a variant thereof, the second cytokine peptide comprises at least a portion of IL-21 or a variant thereof, and the third cytokine peptide comprises at least a portion of IL-23p19 or a variant thereof. In some cases, the first cytokine peptide comprises at least a portion of IL-12 or a variant thereof, the second cytokine peptide comprises at least a portion of IL-12p40 or a variant thereof, and the third cytokine peptide comprises at least a portion of IL-23p19 or a variant thereof. In some cases, the first cytokine peptide comprises at least a portion of IL-15 or a variant thereof, the second cytokine peptide comprises at least a portion of IL-21 or a variant thereof, and the third cytokine peptide comprises at least a portion of IL-12p40 or a variant thereof. In some cases, the first cytokine peptide comprises at least a portion of IL-15 or a variant thereof, the second cytokine peptide comprises at least a portion of IL-21 or a variant thereof, and the third cytokine peptide comprises at least a portion of IL-23p19 or a variant thereof. In some cases, the first cytokine peptide comprises at least a portion of IL-15 or a variant thereof, the second cytokine peptide comprises at least a portion of IL-12p40 or a variant thereof, and the third cytokine peptide comprises at least a portion of IL-23p19 or a variant thereof. In some cases, the first cytokine peptide comprises at least a portion of IL-21 or a variant thereof, the second cytokine peptide comprises at least a portion of IL-12p40 or a variant thereof, and the third cytokine peptide comprises at least a portion of IL-23p19 or a variant thereof.

In any one of the foregoing or related aspects, the glycolipid-attachment signal comprises a glycosylphosphatidylinositol (GPI)-attachment signal. In some cases, the GPI-attachment signal comprises an amino acid sequence that is at least 75%, 80%, 85%, 90%, 95%, 98%, 99%, or 100% identical to the sequence set forth in SEQ ID NO: 200. In some cases, the GPI-attachment signal is encoded by a nucleic acid sequence that is at least 75%, 80%, 85%, 90%, 95%, 98%, 99%, or 100% identical to any of the sequences set forth in SEQ ID NOs: 250-252.

In any one of the foregoing or related aspects, each of the first peptide anchor, the second peptide anchor, and the third peptide anchor independently comprises a transmembrane peptide sequence.

In any one of the foregoing or related aspects, the variant of the first cytokine peptide, the second cytokine peptide, and the third cytokine peptide comprises an alteration, substitution, deletion, addition, or chemical modification of one or more amino acids, one or more unnatural amino acids, or any combination thereof.

In any one of the foregoing or related aspects, each of the first peptide anchor, the second peptide anchor, and the third peptide anchor independently comprises a transmembrane peptide sequence. In some cases, the transmembrane peptide sequence comprises a B7-1 transmembrane amino acid sequence, a B7-2 transmembrane amino acid sequence. B7-H1 transmembrane amino acid sequence. B7-H3 transmembrane amino acid sequence, tumor necrosis factor receptor 2 (TNFR2) transmembrane amino acid sequence, a CD8α transmembrane amino acid sequence, a CD28 transmembrane amino acid sequence, a CD3ζ transmembrane amino acid sequence, a CTLA-4 (CD152) transmembrane amino acid sequence, or a PD-L1 transmembrane amino acid sequence, or any fragments or variants thereof. In some cases, the transmembrane peptide sequence comprises an amino acid sequence that is at least 75%, 80%, 85%, 90%, 95%, 98%, 99%, or 100% identical to any of the sequences set forth in SEQ ID NOs: 203-204. In some cases, the transmembrane peptide sequence is encoded by a nucleic acid sequence that is at least 75%, 80%, 85%, 90%, 95%, 98%, 99%, or 100% identical to any of the sequences set forth in SEQ ID NOs: 253-255.

In any one of the foregoing or related aspects, the nucleic acid molecule described herein, the immune cell described herein, the system described herein, or the cell described herein further comprises a targeting sequence encoding a targeting moiety.

In any one of the foregoing or related aspects, the targeting sequence is linked to the nucleic acid sequence or the exogenous nucleic acid sequence via a nucleic acid sequence encoding a cleavable linker.

In any one of the foregoing or related aspects, the targeting sequence and the first nucleic acid sequence are under control of two different promoters. In any one of the foregoing or related aspects, the targeting sequence and the first nucleic acid sequence are under control of a same promoter. In some cases, the targeting sequence and the second nucleic acid sequence are under control of two different promoters. In some cases, the targeting sequence and the second nucleic acid sequence are under control of a same promoter. In some cases, the targeting sequence and the third nucleic acid sequence are under control of two different promoters. In some cases, the targeting sequence and the third nucleic acid sequence are under control of a same promoter. In some cases, the cleavable linker connects the CAR and the first signal peptide, the second signal peptide and/or the third signal peptide. In some cases, the cleavable linker connects the CAR and the first peptide anchor, the second peptide anchor and/or the third peptide anchor. In some cases, the cleavable linker connects the CAR and the first non-peptide anchor attachment signal, the second non-peptide anchor attachment signal and/or the third non-peptide anchor attachment signal.

In any one of the foregoing or related aspects, each of the first polypeptide, the first protein, the second polypeptide, the second protein, the third polypeptide, and the third protein independently further comprises a peptide linker. In some cases, the peptide linker connects the first cytokine peptide to the first peptide anchor, connects the second cytokine peptide to the second peptide anchor and/or connects the third cytokine peptide to the third peptide anchor. In some cases, the peptide linker connects the first cytokine peptide to the first non-peptide anchor attachment signal, connects the second cytokine peptide to the second non-peptide anchor attachment signal, and/or connects the third cytokine peptide to the third non-peptide anchor attachment signal. In some cases, the peptide linker connects the first peptide anchor, the peptide anchor, and/or the third peptide anchor to the cleavable linker. In some cases, the targeting sequence and the third nucleic acid sequence are present in a same plasmid inside the system. In some cases, the targeting sequence and the third nucleic acid sequence are present in two different plasmids inside the system. In some cases, the first nucleic acid sequence and the third nucleic acid sequence are present in a same plasmid inside the system. In some cases, the first nucleic acid sequence and the third nucleic acid sequence are present in two different plasmids inside the system. In some cases, the second nucleic acid sequence and the third nucleic acid sequence are present in a same plasmid inside the system. In some cases, the second nucleic acid sequence and the third nucleic acid sequence are present in two different plasmids inside the system. In some cases, the targeting sequence and the third nucleic acid sequence are present in a same plasmid inside the cell. In some cases, the targeting sequence and the third nucleic acid sequence are present in two different plasmids inside the cell. In some cases, the first nucleic acid sequence and the third nucleic acid sequence are present in a same plasmid inside the cell. In some cases, the first nucleic acid sequence and the third nucleic acid sequence are present in two different plasmids inside the cell. In some cases, the second nucleic acid sequence and the third nucleic acid sequence are present in a same plasmid inside the cell. In some cases, the second nucleic acid sequence and the third nucleic acid sequence are present in two different plasmids inside the cell. In some cases, the targeting sequence, the first nucleic acid sequence, the second nucleic acid sequence, and the third nucleic acid sequence are present in a genome of the cell.

In any one of the foregoing or related aspects, the targeting moiety comprises a chimeric antigen receptor, a T cell receptor, a B cell receptor, or any combination thereof. In some cases, the targeting sequence is under control of a different promoter as the nucleic acid sequence or the exogenous nucleic acid sequence. In some cases, the targeting sequence is under control of a same promoter as the nucleic acid sequence or the exogenous nucleic acid sequence. In some cases, the targeting sequence encodes a chimeric antigen receptor (CAR). In some cases, the chimeric antigen receptor (CAR) comprises a ligand binding domain, and wherein the ligand binding domain targets CD19, CD20, CD22, CD30, CD33, CD38, CD70, CD123, CD138, CD171, CD5, CD7, MUC1, AFP, CEA, PSCA, PSMA, HER2, EGFR, IL13Ralpha2, GD2, NKG2D, EGFTvIII, CS1, CCL1, BCMA, Mesothelin, ROR1, FLT3, FAP, TAG72, CD38, CD44v6, CEA, EPCAM, B7H3, KIT, IL-13Ralpha2, PRSS21, VEGR2, LewisY, CD24, PDGFR-beta, SSEA-4, AFP, NCAM, Claudin18.2, GPC3, GM3, TGS5, HMWMAA, TEM7R, CLDN6, GPRC5D, CXORF61, CD97, CD179a, ALK, PLAC1, GloboH, NY-BR-1, UPK2, HAVCR1, ADRB3, PANX3, GPR20, LY6K, OR51E2, TRAP, WT1, NY-ESO-1, LAGE-1a, or MAGE-A1. In some cases, the ligand binding domain targets CD19. In some cases, the targeting sequence encodes CAR19. In some cases, the CAR comprises an amino acid sequence that is at least 75%, 80%, 85%, 90%, 95%, 98%, 99%, or 100% identical to any of the sequences set forth in SEQ ID NOs: 4 or 400-407. In some cases, the CAR is encoded by a nucleic acid sequence that is at least 75%, 80%, 85%, 90%, 95%, 98%, 99%, or 100% identical to any of the sequences set forth in SEQ ID NOs: 54 or 450-457.

In any one of the foregoing or related aspects, the targeting sequence is linked to the first nucleic acid sequence or the second nucleic acid sequence via a nucleic acid sequence encoding a cleavable linker. In some cases, the cleavable linker connects the CAR and the first signal peptide and/or the second signal peptide. In some cases, the cleavable linker connects the CAR and the first peptide anchor and/or the second peptide anchor. In some cases, the cleavable linker connects the CAR and the first non-peptide anchor attachment signal and/or the second non-peptide anchor attachment signal.

In any one of the foregoing or related aspects, the cleavable linker comprises P2A peptide, T2A peptide, E2A peptide, F2A peptide, or IRES peptide. In some cases, the cleavable linker is encoded by a nucleic acid sequence having at least 75%, 80%, 85%, 90%, 95%, 98%, 99%, or 100% identical to any of the sequences set forth in SEQ ID NOs: 350-355. In some cases, the cleavable linker connects the CAR and the signal peptide. In some cases, the cleavable linker connects the CAR and the peptide anchor. In some cases, the cleavable linker connects the CAR and the non-peptide anchor attachment signal. In some cases, the cleavable linker comprises an amino acid sequence that is at least 75%, 80%, 85%, 90%, 95%, 98%, 99%, or 100% identical to any of the sequences set forth in SEQ ID NOs: 300-303.

In any one of the foregoing or related aspects, teach of the first polypeptide, the first protein, the second polypeptide, and the second protein independently further comprises a peptide linker. In some cases, the peptide linker connects the first cytokine peptide to the first peptide anchor and/or connects the second cytokine peptide to the second peptide anchor. In some cases, the peptide linker connects the first cytokine peptide to the first non-peptide anchor attachment signal and/or connects the second cytokine peptide to the second non-peptide anchor attachment signal. In some cases, the peptide linker connects the first peptide anchor and/or the second peptide anchor to the cleavable linker. In some cases, the peptide linker connects the first non-peptide anchor attachment signal and/or the second non-peptide anchor attachment signal to the cleavable linker.

In any one of the foregoing or related aspects, the peptide linker comprises a GS linker, a Lr1 linker, or a Lr8 linker. In some cases, the peptide linker comprises an amino acid sequence that is at least 75%, 80%, 85%, 90%, 95%, 98%, 99%, or 100% identical to any of the sequences set forth in SEQ ID NOs: 500, 501, 504, 506, or 507, or the sequence of LE, AS, GSG, or EF. In some cases, the peptide linker is encoded by a nucleic acid sequence that is at least 75%, 80%, 85%, 90%, 95%, 98%, or 99%, or 100% identical to the sequence of ggctccggc, ggaagcgga, gagttc, the sequence of SEQ ID NO: 509, or the sequence of SEQ ID NO: 520.

In any one of the foregoing or related aspects, the nucleic acid molecule is RNA. In any one of the foregoing or related aspects, the nucleic acid molecule is DNA. In some cases, the nucleic acid molecule is a linear RNA. In some cases, the nucleic acid molecule is a circular RNA. In some cases, the nucleic acid molecule is a vector. In some cases, the vector is a viral vector. In some cases, the vector is a retroviral vector, a lentiviral vector, an adenoviral vector, or an adeno-associated viral vector.

In any one of the foregoing or related aspects, the targeting sequence and the first nucleic acid sequence are present in a same plasmid inside the system. In some cases, the targeting sequence and the first nucleic acid sequence are present in two different plasmids inside the system. In some cases, the targeting sequence and the second nucleic acid sequence are present in a same plasmid inside the system. In some cases, the targeting sequence and the second nucleic acid sequence are present in two different plasmids inside the system. In some cases, the first nucleic acid sequence and the second nucleic acid sequence are present in a same plasmid inside the system. In some cases, the first nucleic acid sequence and the second nucleic acid sequence are present in two different plasmids inside the system.

In any one of the foregoing or related aspects, the cell is a bacterial cell, yeast cell, or an insect cell. In some cases, the cell is an immune cell or a tumor cell. In some cases, the immune cell is an engineered immune cell. In some cases, the immune cell is a T cell. In some cases, the immune cell is a tumor infiltrating lymphocyte (TIL). In some cases, the engineered immune cell is a natural killer (NK) cell. In some cases, the targeting sequence and the nucleic acid sequence are present in a same plasmid inside the immune cell. In some cases, the targeting sequence and the nucleic acid sequence are present in two different plasmids inside the immune cell. In some cases, the targeting sequence and the nucleic acid sequence are present in a genome of the immune cell.

In any one of the foregoing or related aspects, the targeting sequence and the first nucleic acid sequence are present in a same plasmid inside the cell. In some cases, the targeting sequence and the first nucleic acid sequence are present in two different plasmids inside the cell. In some cases, the targeting sequence and the second nucleic acid sequence are present in a same plasmid inside the cell. In some cases, the targeting sequence and the second nucleic acid sequence are present in two different plasmids inside the cell. In some cases, the first nucleic acid sequence and the second nucleic acid sequence are present in a same plasmid inside the cell. In some cases, the first nucleic acid sequence and the second nucleic acid sequence are present in two different plasmids inside the cell. In some cases, the targeting sequence, the first nucleic acid sequence, and the second nucleic acid sequence are present in a genome of the cell.

Disclosed herein, in some aspects, is a nucleic acid molecule comprising a nucleic acid sequence that is at least 75%, 80%, 85%, 90%, 95%, 98%, 99%, or 100% identical to any of the sequences set forth in SEQ ID NOs: 510-514, 518-519, 521-522, 527-529, 533, 600-602, 606, 611-617, 625-638, 652-654, 658-666, 676-686, or 698.

Disclosed herein, in some aspects, is a polypeptide comprising an amino acid sequence that is at least 75%, 80%, 85%, 90%, 95%, 98%, 99%, or 100% identical to any of the sequences set forth in SEQ ID NOs: 515-517, 523-526, 530-532, 534, 603-605, 607, 618-624, 639-651, 655-657, 667-675, 687-697, or 699.

Disclosed herein, in some aspects, is a nucleic acid molecule comprising a nucleic acid sequence that encodes the polypeptide of described herein.

Disclosed herein, in some aspects, is a cell comprising the nucleic acid molecule described herein.

Disclosed herein, in some aspects, is a pharmaceutical composition comprising the immune cell described herein or the cell described herein, and a pharmaceutically acceptable excipient or carrier.

Disclosed herein, in some aspects, is a kit, comprising: (a) the immune cell described herein, the cell described herein, or a pharmaceutical composition described herein; and (b) an information material containing instructions for administering a dosage of the immune cell, the cell, or a dosage form of the pharmaceutical composition to a subject.

Disclosed herein, in some aspects, is a method of treating a subject in need thereof, the method comprising administering to the subject the immune cell described herein, the cell described herein, or the pharmaceutical composition described herein. In some cases, the immune cell or the cell is allogeneic to the subject. In some cases, the immune cell or the cell is autologous to the subject. In some cases, the method further comprising obtaining a population of immune cells, and engineering the population of immune cells or a progeny thereof to produce the engineered immune cell. In some cases, the immune cell, the cell, or the pharmaceutical composition treats a cancer in the subject. In some cases, the cancer comprises a solid tumor. In some cases, the cancer comprises leukemia. In some cases, the cancer comprises melanoma. In some cases, the cancer comprises lymphoma. In some cases, the cancer comprises adrenal gland cancer, bladder cancer, bone cancer, brain tumor, breast cancer, cervical cancer, colorectal cancer, esophageal cancer, fallopian tube cancer, gastrointestinal cancer, glioma, glioblastoma, head and neck cancer, hematopoietic malignancy, leukemia, liver cancer, lung cancer, lymphoma, myeloma, nasal cancer, nasopharyngeal cancer, oral cancer, oropharyngeal cancer, ovarian cancer, pancreatic cancer, prostate cancer, sarcoma, stomach cancer, squamous cell lung cancer, testicular cancer, thyroid cancer, uterine cancer, or any combination thereof. In some cases, the subject has not received lymphodepletion prior to the administration.

Disclosed herein, in some aspects, is use of the immune cell described herein, the cell described herein, or the pharmaceutical composition described herein, in the manufacture of a medicament to treat a subject. In some cases, the immune cell or the cell is allogeneic to the subject. In some cases, the immune cell or the cell is autologous to the subject. In some cases, the immune cell or the cell treats a cancer in the subject. In some cases, the cancer comprises a solid tumor. In some cases, the cancer comprises leukemia. In some cases, the cancer comprises melanoma. In some cases, the cancer comprises lymphoma. In some cases, the cancer comprises adrenal gland cancer, bladder cancer, bone cancer, brain tumor, breast cancer, cervical cancer, colorectal cancer, esophageal cancer, fallopian tube cancer, gastrointestinal cancer, glioma, glioblastoma, head and neck cancer, hematopoietic malignancy, leukemia, liver cancer, lung cancer, lymphoma, myeloma, nasal cancer, nasopharyngeal cancer, oral cancer, oropharyngeal cancer, ovarian cancer, pancreatic cancer, prostate cancer, sarcoma, stomach cancer, squamous cell lung cancer, testicular cancer, thyroid cancer, uterine cancer, or any combination thereof.

Disclosed herein, in some aspects, is a method for making an engineered immune cell, comprising introducing the nucleic acid molecule described herein, or the system described herein into an immune cell.

Disclosed herein, in some aspects, is a method for making the immune cell described herein or the cell described herein.

Disclosed herein, in some aspects, is a method for making a pharmaceutical composition, the method comprising combining the immune cell described herein or the cell described herein, with a pharmaceutically acceptable excipient or carrier.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows the cytotoxicity of engineered T cells expressing CAR19+TeIL-2 anchored on cell membrane and the cytotoxicity of engineered T cells expressing CAR19 without TeIL-2 when exposed to tumor cells at an Effector to Target (E:T) ratio of 1:10 for 24 hours.

FIG. 1B shows the proliferation of CD8+ engineered T cells expressing CAR19+TeIL-2 anchored on cell membrane and the proliferation of CD8+ engineered T cells expressing CAR19 without TeIL-2 when exposed to tumor cells for 12 days.

FIG. 2A shows the cytotoxicity of engineered T cells expressing CAR19+TeIL-7 anchored on cell membrane and the cytotoxicity of engineered T cells expressing CAR19 without TeIL-7 when exposed to tumor cells at an E:T ratio of 1:10 for 24 hours.

FIG. 2B shows the proliferation of CD8+ engineered T cells expressing CAR19+TeIL-7 anchored on cell membrane and the proliferation of CD8+ engineered T cells expressing CAR19 without TeIL-7 when exposed to tumor cells for 12 days.

FIG. 3A shows the cytotoxicity of engineered T cells expressing CAR19+TeIL-9 anchored on cell membrane and the cytotoxicity of engineered T cells expressing CAR19 without TeIL-9 when exposed to tumor cells at an E:T ratio of 1:9 for 24 hours.

FIG. 3B shows the proliferation of CD8+ engineered T cells expressing CAR19+TeIL-9 anchored on cell membrane and the proliferation of CD8+ engineered T cells expressing CAR19 without TeIL-9 when exposed to tumor cells for 6 days.

FIG. 4A shows the cytotoxicity of engineered T cells expressing CAR19-TeIL-12p40 anchored on cell membrane and the cytotoxicity of engineered T cells expressing CAR19 without TeIL-12p40 when exposed to tumor cells at an E:T ratio of 1:9 for 24 hours.

FIG. 4B shows the proliferation of CD8+ engineered T cells expressing CAR19-TeIL-12p40 anchored on cell membrane and the proliferation of CD8+ engineered T cells expressing CAR19 without TeIL-12p40 when exposed to tumor cells for 9 days.

FIG. 5A shows the cytotoxicity of engineered T cells expressing CAR19-TeIL-15 anchored on cell membrane and the cytotoxicity of engineered T cells expressing CAR19 without TeIL-15 when exposed to tumor cells at an E:T ratio of 1:9 for 24 hours.

FIG. 5B shows the proliferation of CD8+ engineered T cells expressing CAR19-TeIL-15 anchored on cell membrane and the proliferation of CD8+ engineered T cells expressing CAR19 without TeIL-15 when exposed to tumor cells for 9 days.

FIG. 6A shows the cytotoxicity of engineered T cells expressing CAR19+TeIL-18 anchored on cell membrane and the cytotoxicity of engineered T cells expressing CAR19 without TeIL-18 when exposed to tumor cells at an E:T ratio of 1:9 for 24 hours.

FIG. 6B shows the proliferation of CD8+ engineered T cells expressing CAR19+TeIL-18 anchored on cell membrane and the proliferation of CD8+ engineered T cells expressing CAR19 without TeIL-18 when exposed to tumor cells for 6 days.

FIG. 7A shows the cytotoxicity of engineered T cells expressing CAR19+CAR19-TeIL-21 anchored on cell membrane and the cytotoxicity of engineered T cells expressing CAR19 without TeIL-21 when exposed to tumor cells at an E:T ratio of 1:9 for 24 hours.

FIG. 7B shows the proliferation of CD8+ engineered T cells expressing CAR19+CAR19-TeIL-21 anchored on cell membrane and the proliferation of CD8+ engineered T cells expressing CAR19 without TeIL-21 when exposed to tumor cells for 6 days.

FIG. 8A shows the cytotoxicity of engineered T cells expressing CAR19+TeIL-36γ anchored on cell membrane and the cytotoxicity of engineered T cells expressing CAR19 without TeIL-36γ when exposed to tumor cells at an E:T ratio of 1:9 for 24 hours.

FIG. 8B shows the proliferation of CD8+ engineered T cells expressing CAR19+TeIL-36γ anchored on cell membrane and the proliferation of CD8+ engineered T cells expressing CAR19 without TeIL-36γ when exposed to tumor cells for 6 days.

FIG. 9A shows the cytotoxicity of engineered T cells expressing CAR19-TeIL-12p40+TeIL-7 anchored on cell membrane and the cytotoxicity of engineered T cells expressing CAR19 without TeIL-12p40 or TeIL-7 when exposed to tumor cells at an E:T ratio of 1:9 for 24 hours.

FIG. 9B shows the proliferation of CD8+ engineered T cells expressing CAR19+TeIL-12p40+TeIL-7 anchored on cell membrane and the proliferation of CD8+ engineered T cells expressing CAR19 without TeIL-12p40 or TeIL-7 when exposed tumor cells to 9 days.

FIG. 10A shows the cytotoxicity of engineered T cells expressing CAR19-TeIL-12p40+CAR19-TeIL-15 anchored on cell membrane and the cytotoxicity of engineered T cells expressing CAR19 without TeIL-12p40 or TeIL-15 when exposed to tumor cells at an E:T ratio of 1:9 for 24 hours.

FIG. 10B shows the proliferation of CD8+ engineered T cells expressing CAR19-TeIL-12p40+CAR19-TeIL-15 anchored on cell membrane and the proliferation of CD8+ engineered T cells expressing CAR19 without TeIL-12p40 or TeIL-15 when exposed to tumor cells for 9 days.

FIG. 11A shows the cytotoxicity of engineered T cells expressing CAR19-TeIL-12p40+TeIL-12p40-TeIL-21 anchored on cell membrane and the cytotoxicity of engineered T cells expressing CAR19 without TeIL-12p40 or TeIL-21 when exposed to tumor cells at an E:T ratio of 1:18 for 72 hours.

FIG. 11B shows the proliferation of CD8+ engineered T cells expressing CAR19-TeIL-12p40+TeIL-12p40-TeIL-21 anchored on cell membrane and the proliferation of CD8+ engineered T cells expressing CAR19 without TeIL-12p40 or TeIL-21 when exposed to tumor cells for 9 days.

FIG. 12A shows the cytotoxicity of engineered T cells expressing CAR19-TeIL-15+TeIL-2 anchored on cell membrane and the cytotoxicity of engineered T cells expressing CAR19 without TeIL-15 or TeIL-2 when exposed to tumor cells at an E:T ratio of 1:9 for 24 hours.

FIG. 12B shows the proliferation of CD8+ engineered T cells expressing CAR19-TeIL-15+TeIL-2 anchored on cell membrane and the proliferation of CD8+ engineered T cells expressing CAR19 without TeIL-15 or TeIL-2 when exposed to tumor cells for 9 days.

FIG. 13A shows the cytotoxicity of engineered T cells expressing CAR19-TeIL-15+TeIL-7 anchored on cell membrane and the cytotoxicity of engineered T cells expressing CAR19 without TeIL-15 or TeIL-7 when exposed to tumor cells at an E:T ratio of 1:9 for 24 hours.

FIG. 13B shows the proliferation of CD8+ engineered T cells expressing CAR19-TeIL-15+TeIL-7 anchored on cell membrane and the proliferation of CD8+ engineered T cells expressing CAR19 without TeIL-15 or TeIL-7 when exposed to tumor cells for 6 days.

FIG. 14A shows the cytotoxicity of engineered T cells expressing CAR19+TeIL-15-TeIL-12p40 anchored on cell membrane and the cytotoxicity of engineered T cells expressing CAR19 without TeIL-12p40 or TeIL-15 when exposed to tumor cells at an E:T ratio of 1:18 for 72 hours.

FIG. 14B shows the proliferation of CD8+ engineered T cells expressing CAR19+TeIL-15-TeIL-12p40 anchored on cell membrane and the proliferation of CD8+ engineered T cells expressing CAR19 without TeIL-12p40 or TeIL-15 when exposed to tumor cells for 9 days.

FIG. 15A shows the cytotoxicity of engineered T cells expressing CAR19-TeIL-15+TeIL-15-TeIL-21 anchored on cell membrane and the cytotoxicity of engineered T cells expressing CAR19 without TeIL-15 or TeIL-21 when exposed to tumor cells at an E:T ratio of 1:9 for 24 hours.

FIG. 15B shows the proliferation of CD8+ engineered T cells expressing CAR19-TeIL-15+TeIL-15-TeIL-21 anchored on cell membrane and the proliferation of CD8+ engineered T cells expressing CAR19 without TeIL-15 or TeIL-21 when exposed to tumor cells for 9 days.

FIG. 16A shows the cytotoxicity of engineered T cells expressing CAR19-TeIL-21+TeIL-7 anchored on cell membrane and the cytotoxicity of engineered T cells expressing CAR19 without TeIL-21 or TeIL-7 when exposed to tumor cells at an E:T ratio of 1:9 for 24 hours.

FIG. 16B shows the proliferation of CD8+ engineered T cells expressing CAR19-TeIL-21+TeIL-7 anchored on cell membrane and the proliferation of CD8+ engineered T cells expressing CAR19 without TeIL-21 or TeIL-7 when exposed to tumor cells for 13 days.

FIG. 17A shows the cytotoxicity of engineered T cells expressing CAR19+TeIL-21-TeIL-12p40 anchored on cell membrane and the cytotoxicity of engineered T cells expressing CAR19 without TeIL-21 or TeIL-12p40 when exposed to tumor cells at an E:T ratio of 1:9 for 24 hours.

FIG. 17B shows the proliferation of CD8+ engineered T cells expressing CAR19+TeIL-21-TeIL-12p40 anchored on cell membrane and the proliferation of CD8+ engineered T cells expressing CAR19 without TeIL-21 or TeIL-12p40 when exposed to tumor cells for 9 days.

FIG. 18A shows the cytotoxicity of engineered T cells expressing CAR19-TeIL-21+TeIL-15 anchored on cell membrane and the cytotoxicity of engineered T cells expressing CAR19 without TeIL-21 or TeIL-15 when exposed to tumor cells at an E:T ratio of 1:9 for 24 hours.

FIG. 18B shows the proliferation of CD8+ engineered T cells expressing CAR19-TeIL-21+TeIL-7 anchored on cell membrane and the proliferation of CD8+ engineered T cells expressing CAR19 without TeIL-21 or TeIL-15 when exposed to tumor cells for 13 days.

FIG. 19A shows the cytotoxicity of engineered T cells expressing CAR19+TeIL-2+TeIL-7+TeIL-15 anchored on cell membrane and the cytotoxicity of engineered T cells expressing CAR19 without TeIL-2, TeIL-7, or TeIL-15 when exposed to tumor cells at an E:T ratio of 1:10 for 24 hours.

FIG. 19B shows the proliferation of CD8+ engineered T cells expressing CAR19+TeIL-2+TeIL-7+TeIL-15 anchored on cell membrane and the proliferation of CD8+ engineered T cells expressing CAR19 without TeIL-2, TeIL-7 or TeIL-15 when exposed to tumor cells for 12 days.

FIG. 20A shows the cytotoxicity of engineered T cells expressing CAR19-TeIL-12p40+TeIL-7+TeIL-21 anchored on cell membrane and the cytotoxicity of engineered T cells expressing CAR19 without TeIL-12p40, TeIL-7, or TeIL-21 when exposed to tumor cells at an E:T ratio of 1:9 for 24 hours.

FIG. 20B shows the proliferation of CD8+ engineered T cells expressing CAR19+TeIL-12p40+TeIL-7+TeIL-21 anchored on cell membrane and the proliferation of CD8+ engineered T cells expressing CAR19 without TeIL-12p40, TeIL-7, or TeIL-21 when exposed to tumor cells for 6 days.

FIG. 21A shows the cytotoxicity of engineered T cells expressing CAR19-TeIL-12p40+TeIL-15+TeIL-21 anchored on cell membrane and the cytotoxicity of engineered T cells expressing CAR19 without TeIL-12p40, TeIL-15, or TeIL-21 when exposed to tumor cells at an E:T ratio of 1:18 for 72 hours.

FIG. 21B shows the proliferation of CD8+ engineered T cells expressing CAR19-TeIL-12p40+TeIL-15+TeIL-21 anchored on cell membrane and the proliferation of CD8+ engineered T cells expressing CAR19 without TeIL-12p40, TeIL-15, or TeIL-21 when exposed to tumor cells for 9 days.

FIG. 22A shows the cytotoxicity of engineered T cells expressing CAR19-TeIL-15+TeIL-7+TeIL-21 anchored on cell membrane and the cytotoxicity of engineered T cells expressing CAR19 without TeIL-15, TeIL-7, or TeIL-21 when exposed to tumor cells at an E:T ratio of 1:9 for 24 hours.

FIG. 22B shows the proliferation of CD8+ engineered T cells expressing CAR19-TeIL-15+TeIL-7+TeIL-21 anchored on cell membrane and the proliferation of CD8+ engineered T cells expressing CAR19 without TeIL-15, TeIL-7, or TeIL-21 when exposed to tumor cells for 6 days.

FIG. 23A shows the cytotoxicity of engineered T cells expressing CAR19-TeIL-15+TeIL-12p40-TeIL-21 anchored on cell membrane and the cytotoxicity of engineered T cells expressing CAR19 without TeIL-15, TeIL-12p40, or TeIL-21 when exposed to tumor cells at an E:T ratio of 1:9 for 24 hours.

FIG. 23B shows the proliferation of CD8+ engineered T cells expressing CAR19-TeIL-15+TeIL-12p40-TeIL-21 anchored on cell membrane and the proliferation of CD8+ engineered T cells expressing CAR19 without TeIL-15, TeIL-12p40, or TeIL-21 when exposed to tumor cells for 9 days.

FIG. 24A shows the cytotoxicity of engineered T cells expressing CAR19-TeIL-15+TeIL-21-TeIL-12p40 anchored on cell membrane and the cytotoxicity of engineered T cells expressing CAR19 without TeIL-15, TeIL-21, or TeIL-12p40 when exposed to tumor cells at an E:T ratio of 1:9 for 24 hours.

FIG. 24B shows the proliferation of CD8+ engineered T cells expressing CAR19-TeIL-15+TeIL-21-TeIL-12p40 anchored on cell membrane and the proliferation of CD8+ engineered T cells expressing CAR19 without TeIL-15, TeIL-21, or TeIL-12p40 when exposed to tumor cells for 9 days.

FIG. 25A shows the cytotoxicity of engineered T cells expressing CAR19-TeIL-21+TeIL-7+TeIL-15 anchored on cell membrane and the cytotoxicity of engineered T cells expressing CAR19 without TeIL-21, TeIL-7, or TeIL-15 when exposed to tumor cells at an E:T ratio of 1:9 for 24 hours.

FIG. 25B shows the proliferation of CD8+ engineered T cells expressing CAR19-TeIL-21+TeIL-7+TeIL-15 anchored on cell membrane and the proliferation of CD8+ engineered T cells expressing CAR19 without TeIL-21, TeIL-7, or TeIL-15 when exposed to tumor cells for 13 days.

FIG. 26A shows the cytotoxicity of engineered T cells expressing CAR19-TeIL-21+TeIL-12p40+TeIL-15 anchored on cell membrane and the cytotoxicity of engineered T cells expressing CAR19 without TeIL-21, TeIL-12p40, or TeIL-15 when exposed to tumor cells at an E:T ratio of 1:9 for 24 hours.

FIG. 26B shows the proliferation of CD8+ engineered T cells expressing CAR19-TeIL-21+TeIL-12p40+TeIL-15 anchored on cell membrane and the proliferation of CD8+ engineered T cells expressing CAR19 without TeIL-21, TeIL-12p40, or TeIL-15 when exposed to tumor cells for 13 days.

FIG. 27A shows the cytotoxicity of engineered T cells expressing CAR19+IL-12p40-Lr1-Ar1-E2A-IL-15-Lr1-Ar2 anchored on cell membrane and the cytotoxicity of engineered T cells expressing CAR19 without IL-12p40-Lr1-Ar1-E2A-IL-15-Lr1-Ar2 when exposed to tumor cells at an E:T ratio of 1:3 for 72 hours.

FIG. 27B shows the proliferation of CD8+ engineered T cells expressing CAR19+IL-12p40-Lr1-Ar1-E2A-IL-15-Lr1-Ar2 anchored on cell membrane and the proliferation of CD8+ engineered T cells expressing CAR19 without IL-12p40-Lr1-Ar1-E2A-IL-15-Lr1-Ar2 when exposed to tumor cells for 12 days.

FIG. 28A shows the cytotoxicity of engineered T cells expressing CAR19+IL-12p40-Lr1-Ar1-F2A-IL-15-Lr1-Ar2 anchored on cell membrane and the cytotoxicity of engineered T cells expressing CAR19 without IL-12p40-Lr1-Ar1-F2A-IL-15-Lr1-Ar2 when exposed to tumor cells at an E:T ratio of 1:3 for 72 hours.

FIG. 28B shows the proliferation of CD8+ engineered T cells expressing CAR19+IL-12p40-Lr1-Ar1-F2A-IL-15-Lr1-Ar2 anchored on cell membrane and the proliferation of CD8+ engineered T cells expressing CAR19 without IL-12p40-Lr1-Ar1-F2A-IL-15-Lr1-Ar2 when exposed to tumor cells for 12 days.

FIG. 29A shows the cytotoxicity of engineered T cells expressing CAR19+IL-12p40-Lr1-Ar1-P2A-IL-15-Lr1-Ar2 anchored on cell membrane and the cytotoxicity of engineered T cells expressing CAR19 without IL-12p40-Lr1-Ar1-P2A-IL-15-Lr1-Ar2 when exposed to tumor cells at an E:T ratio of 1:3 for 72 hours.

FIG. 29B shows the proliferation of CD8+ engineered T cells expressing CAR19+IL-12p40-Lr1-Ar1-P2A-IL-15-Lr1-Ar2 anchored on cell membrane and the proliferation of CD8+ engineered T cells expressing CAR19 without IL-12p40-Lr1-Ar1-P2A-IL-15-Lr1-Ar2 when exposed to tumor cells for 12 days.

FIG. 30A shows the cytotoxicity of engineered T cells expressing CAR19+IL-12p40-Lr1-Ar1-T2A-IL-15-Lr1-Ar2 anchored on cell membrane and the cytotoxicity of engineered T cells expressing CAR19 without IL-12p40-Lr1-Ar1-T2A-IL-15-Lr1-Ar2 when exposed to tumor cells at an E:T ratio of 1:3 for 72 hours.

FIG. 30B shows the proliferation of CD8+ engineered T cells expressing CAR19+IL-12p40-Lr1-Ar1-T2A-IL-15-Lr1-Ar2 anchored on cell membrane and the proliferation of CD8+ engineered T cells expressing CAR19 without IL-12p40-Lr1-Ar1-T2A-IL-15-Lr1-Ar2 when exposed to tumor cells for 12 days.

FIG. 31A shows the cytotoxicity of engineered T cells expressing CAR19+IL-12p40-Lr1-Ar2-T2A-IL-15-Lr1-Ar1 anchored on cell membrane and the cytotoxicity of engineered T cells expressing CAR19 without IL-12p40-Lr1-Ar2-T2A-IL-15-Lr1-Ar1 when exposed to tumor cells at an E:T ratio of 1:3 for 72 hours.

FIG. 31B shows the proliferation of CD8+ engineered T cells expressing CAR19+IL-12p40-Lr1-Ar2-T2A-IL-15-Lr1-Ar1 anchored on cell membrane and the proliferation of CD8+ engineered T cells expressing CAR19 without IL-12p40-Lr1-Ar2-T2A-IL-15-Lr1-Ar1 when exposed to tumor cells for 12 days.

FIG. 32A shows the cytotoxicity of engineered T cells expressing CAR19+IL-12p40-Lr1-Ar2-T2A-IL-15-Lr1-Ar2 anchored on cell membrane and the cytotoxicity of engineered T cells expressing CAR19 without IL-12p40-Lr1-Ar2-T2A-IL-15-Lr1-Ar2 when exposed to tumor cells at an E:T ratio of 1:3 for 72 hours.

FIG. 32B shows the proliferation of CD8+ engineered T cells expressing CAR19+IL-12p40-Lr1-Ar2-T2A-IL-15-Lr1-Ar2 anchored on cell membrane and the proliferation of CD8+ engineered T cells expressing CAR19 without IL-12p40-Lr1-Ar2-T2A-IL-15-Lr1-Ar2 when exposed to tumor cells for 12 days.

FIG. 33A shows the cytotoxicity of engineered T cells expressing CAR19+IL-12p40-Lr8-Ar1-E2A-IL-15-Lr1-Ar2 anchored on cell membrane and the cytotoxicity of engineered T cells expressing CAR19 without IL-12p40-Lr8-Ar1-E2A-IL-15-Lr1-Ar2 when exposed to tumor cells at an E:T ratio of 1:3 for 72 hours when exposed to tumor cells for 12 days and 9 days, respectively.

FIG. 33B shows the proliferation of CD8+ engineered T cells expressing CAR19+IL-12p40-Lr8-Ar1-E2A-IL-15-Lr1-Ar2 anchored on cell membrane and the proliferation of CD8+ engineered T cells expressing CAR19 without IL-12p40-Lr8-Ar1-E2A-IL-15-Lr1-Ar2 when exposed to tumor cells for 12 days.

FIG. 34 shows the cytotoxicity of engineered T cells expressing CAR19-TeIL-4 anchored on cell membrane and the cytotoxicity of engineered T cells expressing CAR19 without TeIL-4 when exposed to tumor cells at an E:T ratio of 1:8 and 1:16 for 72 hours.

FIG. 35A shows the proliferation of CD4+ engineered T cells expressing CAR19-TeIL-4 anchored on cell membrane and the proliferation of CD4+ engineered T cells expressing CAR19 without TeIL-4 when exposed to tumor cells at an E:T ratio of 1:8 for 12 days.

FIG. 35B shows the proliferation of CD8+ engineered T cells expressing CAR19-TeIL-4 anchored on cell membrane and the proliferation of engineered CD8+ T cells expressing CAR19 without TeIL-4 when exposed to tumor cells at an E:T ratio of 1:8 for 12 days.

FIG. 36A shows the proliferation of engineered CD4+ T cells expressing CAR19-TeIL-4 anchored on cell membrane and the proliferation of CD4+ engineered T cells expressing CAR19 without TeIL-4 when exposed to tumor cells at an E:T ratio of 1:16 for 12 days.

FIG. 36B shows the proliferation of CD8+ engineered T cells expressing CAR19-TeIL-4 anchored on cell membrane and the proliferation of CD8+ engineered T cells expressing CAR19 without TeIL-4 when exposed to tumor cells at an E:T ratio of 1:16 for 12 days.

FIG. 37 shows the continuous cytotoxicity of engineered T cells expressing CAR19-TeIL-10 anchored on cell membrane, engineered T cells expressing soluble CAR19-sIL-10, and engineered T cells expressing CAR19 without TeIL-10 or sIL-10 when continuously exposed to tumor cells for 45 days.

FIG. 38A shows the continuous proliferation of CD8+ engineered T cells expressing CAR19-TeIL-10 anchored on cell membrane. CD8+ engineered T cells expressing soluble CAR19-sIL-10, and CD8+ engineered T cells expressing CAR19 without TeIL-10 or sIL-10 when continuously exposed to tumor cells for 51 days.

FIG. 38B shows the continuous proliferation of CD4+ engineered T cells expressing CAR19-TeIL-10 anchored on cell membrane. CD4+ engineered T cells expressing soluble CAR19-sIL-10, and CD8+ engineered T cells expressing CAR19 without TeIL-10 or sIL-10 when continuously exposed to tumor cells for 51 days.

FIG. 39 shows the tumor-killing ability of engineered T cells expressing CAR19-TeIL-4 and engineered T cells expressing CAR19-TeIL-4+TeIL-15. Fluorescence intensity (Total Flux p/s) was used to indicate tumor burden.

### DETAILED DESCRIPTION

Disclosed herein, in some aspects, are (a) polypeptides comprising a cytokine peptide and an anchoring structure. (b) nucleic acids encoding the polypeptides. (c) proteins processed from the polypeptides, and (d) cells (e.g., immune cells) expressing the polypeptides ((a)-(d) hereinafter collectively “single cytokine-anchor materials”). In some embodiments, the anchoring structure is capable of attaching the cytokine to a surface of a cell comprising a nucleic acid molecule encoding the polypeptides. In some embodiments, the cytokine peptide comprises at least a portion of IL-2, IL-4, IL-7, IL-9, IL-10, IL-18, IL-23, IL-27, IL-36γ, IL-23p19, or IL-1α, or a variant thereof. In some cases, the cytokine peptide is at least a portion of IL-12p40. In some cases, the cytokine peptide is at least a portion of IL-12p30). In some cases, the cytokine peptide comprises IL-12p40 and does not comprise IL-12p35. In some cases, immune cells comprising the polypeptides described herein does not comprise a stimulus response element (SRE) derived from PDE5. In some cases, immune cells comprising the polypeptides described herein does not comprise a stimulus response element (SRE). In some embodiments, the polypeptide also comprises a signal peptide. In some embodiments, the anchoring structure is a peptide anchor. In some embodiments, the anchoring structure is a non-peptide anchor attachment signal that is later replaced by a non-peptide anchor during protein processing. In some embodiments, the nucleic acid molecule further comprises a targeting sequence. In some embodiments, the targeting sequence encodes a chimeric antigen receptor (CAR), a T cell receptor, a B cell receptor, or any combination thereof. In some embodiments, the targeting sequence encodes a CAR comprising a recognition region that targets CD19. In some embodiments, the targeting sequence encodes a CAR19 (e.g., as described in Table 5A). In some embodiments, the CAR19 and the polypeptide are linked via a cleavable linker. In some embodiments, the signal peptide directs the polypeptide to a surface of the cell. In some embodiments, the peptide anchor comprises a transmembrane peptide sequence. In some embodiments, the non-peptide anchor attachment signal is replaced by a non-peptide anchor, such as a lipid anchor. In some embodiments, the non-peptide anchor attachment signal comprises a glycosylphosphatidylinositol (GPI)-attachment signal. In some embodiments, the non-peptide anchor is a GPI anchor. In some embodiments, the peptide anchor is attached to a membrane of the cell. In some embodiments, the non-peptide anchor is attached to the membrane a cell. In some embodiments, the cleavable linker comprises P2A peptide, T2A peptide, E2A peptide, F2A peptide, or IRES peptide. In some embodiments, the nucleic acid molecule is a vector, such as a retroviral vector, a lentiviral vector, an adenoviral vector, or an adeno-associated viral vector. In some embodiments, the cells comprise immune cells or tumor cells. In some embodiments, the cells comprise engineered immune cells, such as a T cell or a natural killer (NK) cell. In some embodiments, cytotoxicity of said immune cell is increased relative to a comparable immune cell that does not comprise the nucleic acid sequence encoding the cytokine peptide and the anchoring structure (e.g., as shown in Example D). In some embodiments, a population of said immune cells proliferates for a longer period of time relative to a population of comparable immune cells that does not comprise the nucleic acid sequence encoding the cytokine peptide and the anchoring structure (e.g., as shown in Example E). In some cases, the polypeptide comprises an amino acid sequence that is at least 75%, 80%, 85%, 90%, 95%, 98%, or 99%, or 100% identical to any of the sequences set forth in SEQ ID NOs: 515-517, 523-526, 607, 618-624 or 667-675. In some cases, the nucleic acid comprises a nucleic acid sequence that is at least 75%, 80%, 85%, 90%, 95%, 98%, or 99%, or 100% identical to any of the sequences set forth in SEQ ID NOs: 510-514, 518-519, 521-522, 606, 611-617 or 658-666.

Disclosed herein, in some aspects, are (a) systems comprising a first polypeptide and a different second polypeptide, wherein the first polypeptide comprises a first cytokine peptide and a first anchoring structure and the second polypeptide comprises a second cytokine peptide and a second anchoring structure. (b) systems comprising a protein comprising the first polypeptide and a protein comprising the second polypeptide. (c) systems comprising nucleic acids encoding the first polypeptide and nucleic acids encoding the second polypeptide. (d) nucleic acid molecules encoding the first polypeptide and the second polypeptide. (c) cells (e.g., immune cells) expressing the first polypeptide and the different second polypeptide ((a)-(e) hereinafter collectively “two cytokine-anchor materials”). In some embodiments, the nucleic acid molecule encoding the first polypeptide and the second polypeptide comprises a first nucleic acid sequence encoding the first polypeptide and a second nucleic acid sequence encoding the second polypeptide. In some embodiments, the nucleic acid encoding the first polypeptide and the nucleic acid encoding the second polypeptide are different. In some embodiments, the first cytokine peptide is a proinflammatory cytokine peptide, and the second cytokine peptide is an anti-inflammatory cytokine peptide. In some embodiments, the first anchoring structure is capable of attaching the first cytokine to a surface of a cell comprising the nucleic acid molecule. In some embodiments, the second anchoring structure is capable of attaching the second cytokine to the surface of the cell comprising the nucleic acid molecule. In some embodiments, each of the first cytokine peptide and the second cytokine peptide independently comprises at least a portion of IL-2, IL-4, IL-7, IL-9, IL-10, IL-12, IL-12p40, IL-18, IL-21, IL-23, IL-27, IL-36γ, IL-23p19, or IL-1α, or a variant thereof. In other embodiments, each of the first cytokine peptide and the second cytokine peptide independently comprises at least a portion of IL-2, IL-4, IL-7, IL-9, IL-10, IL-12, IL-12p40, IL-18, IL-15, IL-23, IL-27, IL-36γ, IL-23p19, or IL-1α, or a variant thereof. In some embodiments, the first polypeptide comprises a first signal peptide. In some embodiments, the second polypeptide comprises a second signal peptide. In some embodiments, the first anchoring structure is peptide anchor. In some embodiments, the first anchoring structure is a non-peptide anchor attachment signal which is replaced by a non-peptide anchor during protein processing. In some embodiments, the second anchoring structure is peptide anchor. In some embodiments, the second anchoring structure is a non-peptide anchor attachment signal which is replaced by a non-peptide anchor during processing. In some embodiments, the nucleic acid molecule further comprises a targeting sequence. In some embodiments, the targeting sequence encodes a chimeric antigen receptor (CAR), a T cell receptor, a B cell receptor, or any combination thereof. In some embodiments, the targeting sequence encodes a CAR comprising a recognition region that targets CD19. In some embodiments, the targeting sequence encodes a CAR19 (e.g., as described in Table 5A). In some embodiments, the CAR19 and the first polypeptide are linked via a cleavable linker. In some embodiments, the CAR19 and the second polypeptide are linked via a cleavable linker. In some embodiments, the first polypeptide and the second polypeptide are linked via a cleavable linker. In some embodiments, the signal peptide directs the polypeptide to a surface of the cell. In some embodiments, the peptide anchor comprises a transmembrane peptide sequence. In some embodiments, the non-peptide anchor attachment signal is replaced by a non-peptide anchor, such as a lipid anchor. In some embodiments, the non-peptide anchor attachment signal comprises a glycosylphosphatidylinositol (GPI)-attachment signal. In some embodiments, the non-peptide anchor is a GPI anchor. In some embodiments, the peptide anchor is attached to a membrane of the cell. In some embodiments, the non-peptide anchor is attached to the membrane a cell. In some embodiments, the cleavable linker comprises P2A peptide, T2A peptide, E2A peptide, F2A peptide, or IRES peptide. In some embodiments, the nucleic acid molecule is a vector, such as a retroviral vector, a lentiviral vector, an adenoviral vector, or an adeno-associated viral vector. In some embodiments, the cells comprise immune cells or tumor cells. In some embodiments, the cells comprise engineered immune cells, such as a T cell or a natural killer (NK) cell. In some embodiments, cytotoxicity of said immune cell is increased relative to a comparable immune cell that does not comprise the nucleic acid sequence encoding the cytokine peptide and the anchoring structure (e.g., as shown in Example D). In some embodiments, a population of said immune cells proliferates for a longer period of time relative to a population of comparable immune cells that does not comprise the nucleic acid sequence encoding the cytokine peptide and the anchoring structure (e.g., as shown in Example E). In some cases, the first polypeptide comprises an amino acid sequence that is at least 75%, 80%, 85%, 90%, 95%, 98%, or 99%, or 100% identical to any of the sequences set forth in SEQ ID NOs: 515-517, 523-526, 607, 618-624 or 667-675. In some cases, the first nucleic acid sequence comprises a nucleic acid sequence that is at least 75%, 80%, 85%, 90%, 95%, 98%, or 99%, or 100% identical to any of the sequences set forth in SEQ ID NOs: 510-514, 518-519, 521-522, 606, 611-617 or 658-666. In some cases, the second polypeptide comprises an amino acid sequence that is at least 75%, 80%, 85%, 90%, 95%, 98%, or 99%, or 100% identical to any of the sequences set forth in SEQ ID NOs: 515-517, 523-526, 607, 618-624 or 667-675. In some cases, the second nucleic acid sequence comprises a nucleic acid sequence that is at least 75%, 80%, 85%, 90%, 95%, 98%, or 99%, or 100% identical to any of the sequences set forth in SEQ ID NOs: 510-514, 518-519, 521-522, 606, 611-617 or 658-666. In some cases, the nucleic acid molecule comprises a nucleic acid sequence that is at least 75%, 80%, 85%, 90%, 95%, 98%, or 99%, or 100% identical to any of the sequences set forth in SEQ ID NOs: 527-529, 533, 600-602, 625-638 or 676-686. In some cases, the nucleic acid molecule encodes an amino acid sequence that is at least 75%, 80%, 85%, 90%, 95%, 98%, or 99%, or 100% identical to any of the sequences set forth in SEQ ID NOs: 530-532, 534, 603-605, 639-651, or 687-697. In some cases, the anti-inflammatory cytokine peptide comprises at least a portion of IL-4, IL-10, or IL-27, or a variant thereof. In some cases, the proinflammatory cytokine peptide comprises at least a portion of IL-2, IL-7, IL-9, IL-12, IL-12p40, IL-15, IL-18, IL-21, IL-23, IL-36γ, IL-23p19, or IL-1α, or a variant thereof. In some cases, the anti-inflammatory cytokine peptide comprises at least a portion of IL-4 or a variant thereof, and the proinflammatory cytokine peptide comprises at least a portion of IL-2, IL-7, IL-9, IL-12, IL-12p40, IL-15, IL-18, IL-21, IL-23, IL-27, IL-36γ, IL-23p19, or IL-1α or a variant thereof. In some cases, the anti-inflammatory cytokine peptide comprises an amino acid sequence that is at least 75%, 80%, 85%, 90%, 95%, 98%, 99%, or 100% identical to the sequence set forth in SEQ ID NO: 101, 104, or 109, and wherein the proinflammatory cytokine peptide comprises an amino acid sequence that is at least 75%, 80%, 85%, 90%, 95%, 98%, 99%, or 100% identical to any one of the sequences set forth in SEQ ID NOs: 100, 102, 103, 105-108, or 110-112. In some cases, the anti-inflammatory cytokine peptide is encoded by a nucleic acid sequence that is at least 75%, 80%, 85%, 90%, 95%, 98%, 99%, or 100% identical to the sequence set forth in SEQ ID NO: 151, 163, 154, 164, or 159, and wherein the proinflammatory cytokine peptide is encoded by a nucleic acid sequence that is at least 75%, 80%, 85%, 90%, 95%, 98%, 99%, or 100% identical to any one of the sequences set forth in SEQ ID NOs: 150, 152, 153, 155-158, or 160-162. In some cases, the anti-inflammatory cytokine peptide comprises an amino acid sequence that is at least 75%, 80%, 85%, 90%, 95%, 98%, 99%, or 100% identical to the sequence set forth in SEQ ID NO: 101, and wherein the proinflammatory cytokine peptide comprises an amino acid sequence that is at least 75%, 80%, 85%, 90%, 95%, 98%, 99%, or 100% identical to any one of the sequences set forth in SEQ ID NOs: 100, 102, 103, 105-108, or 110-112. In some cases, the anti-inflammatory cytokine peptide is encoded by a nucleic acid sequence that is at least 75%, 80%, 85%, 90%, 95%, 98%, 99%, or 100% identical to the sequence set forth in SEQ ID NO: 151 or 163, and wherein the proinflammatory cytokine peptide is encoded by a nucleic acid sequence that is at least 75%, 80%, 85%, 90%, 95%, 98%, 99%, or 100% identical to any one of the sequences set forth in SEQ ID NOs: 150, 152, 153, 155-158, or 160-162. In some cases, the first cytokine peptide comprises at least a portion of IL-4 or a variant thereof, and wherein the second cytokine peptide comprises at least a portion of IL-2, IL-7, IL-9, IL-10, IL-12, IL-12p40, IL-15, IL-18, IL-21, IL-23, IL-27, IL-36γ, IL-23p19, or IL-1α or a variant thereof. In some cases, the first cytokine peptide comprises an amino acid sequence that is at least 75%, 80%, 85%, 90%, 95%, 98%, 99%, or 100% identical to the sequence set forth in SEQ ID NO: 101, and the second cytokine peptide comprises an amino acid sequence that is at least 75%, 80%, 85%, 90%, 95%, 98%, 99%, or 100% identical to any one of the sequences set forth in SEQ ID NOs: 100 or 102-112. In some cases, the first cytokine peptide is encoded by a nucleic acid sequence that is at least 75%, 80%, 85%, 90%, 95%, 98%, 99%, or 100% identical to the sequence set forth in SEQ ID NO: 151 or 163, and the second cytokine peptide is encoded by a nucleic acid sequence that is at least 75%, 80%, 85%, 90%, 95%, 98%, 99%, or 100% identical to any one of the sequences set forth in SEQ ID NOs: 150, 152-162, or 164. In some cases, the first cytokine peptide comprises at least a portion of IL-4 or a variant thereof, and wherein the second cytokine peptide comprises at least a portion of IL-2, IL-7, IL-9, IL-10, IL-12, IL-12p40, IL-15, IL-18, IL-23, IL-27, IL-36γ, IL-23p19, or IL-1α or a variant thereof. In some cases, the first cytokine peptide comprises an amino acid sequence that is at least 75%, 80%, 85%, 90%, 95%, 98%, 99%, or 100% identical to the sequence set forth in SEQ ID NO: 101, and wherein the second cytokine peptide comprises an amino acid sequence that is at least 75%, 80%, 85%, 90%, 95%, 98%, 99%, or 100% identical to any one of the sequences set forth in SEQ ID NOs: 100, 102-107, or 109-112. In some cases, the first cytokine peptide is encoded by a nucleic acid sequence that is at least 75%, 80%, 85%, 90%, 95%, 98%, 99%, or 100% identical to the sequence set forth in SEQ ID NO: 151 or 163, and wherein the second cytokine peptide is encoded by a nucleic acid sequence that is at least 75%, 80%, 85%, 90%, 95%, 98%, 99%, or 100% identical to any one of the sequences set forth in SEQ ID NOs: 150, 152-157, 159-162, or 164. In some cases, the first cytokine peptide comprises at least a portion of IL-4 or a variant thereof, and wherein the second cytokine peptide comprises at least a portion of IL-2, IL-7, IL-9, IL-10, IL-12, IL-12p40, IL-18, IL-21, IL-23, IL-27, IL-36γ, IL-23p19, or IL-1α or a variant thereof. In some cases, the first cytokine peptide comprises an amino acid sequence that is at least 75%, 80%, 85%, 90%, 95%, 98%, 99%, or 100% identical to the sequence set forth in SEQ ID NO: 101, and wherein the second cytokine peptide comprises an amino acid sequence that is at least 75%, 80%, 85%, 90%, 95%, 98%, 99%, or 100% identical to any one of the sequences set forth in SEQ ID NOs: 100, 102-105, or 107-112. In some cases, the first cytokine peptide is encoded by a nucleic acid sequence that is at least 75%, 80%, 85%, 90%, 95%, 98%, 99%, or 100% identical to the sequence set forth in SEQ ID NO: 151 or 163, and wherein the second cytokine peptide is encoded by a nucleic acid sequence that is at least 75%, 80%, 85%, 90%, 95%, 98%, 99%, or 100% identical to any one of the sequences set forth in SEQ ID NOs: 150, 152-155, 157-162, or 164.

Disclosed herein, in some aspects, are (a) systems comprising a first polypeptide, a second polypeptide, and a third polypeptide, wherein the first polypeptide comprises a first cytokine peptide and a first anchor structure, the second polypeptide comprises a second cytokine peptide and a second anchor structure, and the third polypeptide comprises a third cytokine peptide and a third anchoring structure and wherein the first polypeptide, the second polypeptide, and the third polypeptide are different. (b) systems that comprise a protein comprising the first polypeptide, a protein comprising the second polypeptide, and a protein comprising the third polypeptide. (c) systems that comprise nucleic acids encoding the first polypeptide, nucleic acids encoding the second polypeptide, and nucleic acids encoding the third polypeptide, nucleic acids encoding any combination of the first polypeptide, the second polypeptide, and the third polypeptide. (d) nucleic acid molecules encoding the first polypeptide, the second polypeptide, and the third polypeptide. (e) cells (e.g., immune cells) expressing the first polypeptide, the second polypeptide, and the third polypeptide. ((a)-(e) hereinafter collectively “three cytokine-anchor materials”). In some embodiments, the nucleic acid molecule encoding the first polypeptide, the second polypeptide, and the third polypeptide comprises a first nucleic acid sequence encoding the first polypeptide, a second nucleic acid sequence encoding the second polypeptide, and a third nucleic acid sequence encoding the third polypeptide. In some embodiments, the first anchoring structure is capable of attaching the first cytokine to a surface of a cell comprising the nucleic acid molecule. In some embodiments, the second anchoring structure is capable of attaching the second cytokine to the surface of the cell comprising the nucleic acid molecule. In some embodiments, the third anchoring structure is capable of attaching the second cytokine to the surface of the cell comprising the nucleic acid molecule. In some embodiments, each of the first cytokine peptide, the second cytokine peptide, and the third cytokine peptide independently comprises at least a portion of IL-2, IL-4, IL-7, IL-9, IL-10, IL-12, IL-12p40, IL-15, IL-18, IL-21, IL-23, IL-27, IL-36γ, IL-23p19, or IL-1α, or a variant thereof. In some embodiments, the first polypeptide comprises a first signal peptide. In some embodiments, the second polypeptide comprises a second signal peptide. In some embodiments, the third polypeptide comprises a third signal peptide. In some embodiments, the first anchoring structure is peptide anchor. In some embodiments, the first anchoring structure is a non-peptide anchor attachment signal which is replaced by a non-peptide anchor during protein processing. In some embodiments, the second anchoring structure is peptide anchor. In some embodiments, the second anchoring structure is a non-peptide anchor attachment signal which is replaced by a non-peptide anchor during processing. In some embodiments, the third anchoring structure is peptide anchor. In some embodiments, the third anchoring structure is a non-peptide anchor attachment signal which is replaced by a non-peptide anchor during processing. In some embodiments, the nucleic acid molecule further comprises a targeting sequence. In some embodiments, the targeting sequence encodes a chimeric antigen receptor (CAR), a T cell receptor, a B cell receptor, or any combination thereof. In some embodiments, the targeting sequence encodes a CAR comprising a recognition region that targets CD19. In some embodiments, the targeting sequence encodes a CAR19 (e.g., as described in Table 5A). In some embodiments, the CAR19 and the first polypeptide are linked via a cleavable linker. In some embodiments, the CAR19 and the second polypeptide are linked via a cleavable linker. In some embodiments, the CAR19 and the third polypeptide are linked via a cleavable linker. In some embodiments, the first polypeptide and the second polypeptide are linked via a cleavable linker. In some embodiments, the second polypeptide and the third polypeptide are linked via a cleavable linker. In some embodiments, the first polypeptide and the third polypeptide are linked via a cleavable linker. In some embodiments, the signal peptide directs the polypeptide to a surface of the cell. In some embodiments, the peptide anchor comprises a transmembrane peptide sequence. In some embodiments, the non-peptide anchor attachment signal is replaced by a non-peptide anchor, such as a lipid anchor. In some embodiments, the non-peptide anchor attachment signal comprises a glycosylphosphatidylinositol (GPI)-attachment signal. In some embodiments, the non-peptide anchor is a GPI anchor. In some embodiments, the peptide anchor is attached to a membrane of the cell. In some embodiments, the non-peptide anchor is attached to the membrane a cell. In some embodiments, the cleavable linker comprises P2A peptide, T2A peptide, E2A peptide, F2A peptide, or IRES peptide. In some embodiments, the nucleic acid molecule is a vector, such as a retroviral vector, a lentiviral vector, an adenoviral vector, or an adeno-associated viral vector. In some embodiments, the cells comprise immune cells or tumor cells. In some embodiments, the cells comprise engineered immune cells, such as a T cell or a natural killer (NK) cell. In some embodiments, cytotoxicity of said immune cell is increased relative to a comparable immune cell that does not comprise the nucleic acid sequence encoding the cytokine peptide and the anchoring structure (e.g., as shown in Example D). In some embodiments, a population of said immune cells proliferates for a longer period of time relative to a population of comparable immune cells that does not comprise the nucleic acid sequence encoding the cytokine peptide and the anchoring structure (e.g., as shown in Example E). In some cases, the first polypeptide comprises an amino acid sequence that is at least 75%, 80%, 85%, 90%, 95%, 98%, or 99%, or 100% identical to any of the sequences set forth in SEQ ID NOs: 515-517, 523-526, 607, 618-624 or 667-675. In some cases, the first nucleic acid sequence comprises a nucleic acid sequence that is at least 75%, 80%, 85%, 90%, 95%, 98%, or 99%, or 100% identical to any of the sequences set forth in SEQ ID NOs: 510-514, 518-519, 606, 521-522, 611-617 or 658-666. In some cases, the second polypeptide comprises an amino acid sequence that is at least 75%, 80%, 85%, 90%, 95%, 98%, or 99%, or 100% identical to any of the sequences set forth in SEQ ID NOs: 515-517, 523-526, 607, 618-624 or 667-675. In some cases, the second nucleic acid sequence comprises a nucleic acid sequence that is at least 75%, 80%, 85%, 90%, 95%, 98%, or 99%, or 100% identical to any of the sequences set forth in SEQ ID NOs: 510-514, 518-519, 606, 521-522, 611-617 or 658-666. In some cases, the third polypeptide comprises an amino acid sequence that is at least 75%, 80%, 85%, 90%, 95%, 98%, or 99%, or 100% identical to any of the sequences set forth in SEQ ID NOs: 515-517, 523-526, 607, 618-624 or 667-675. In some cases, the third nucleic acid sequence comprises a nucleic acid sequence that is at least 75%, 80%, 85%, 90%, 95%, 98%, or 99%, or 100% identical to any of the sequences set forth in SEQ ID NOs: 510-514, 518-519, 606, 521-522, 611-617 or 658-666. In some cases, the nucleic acid molecule comprises a nucleic acid sequence that is at least 75%, 80%, 85%, 90%, 95%, 98%, or 99%, or 100% identical to any of the sequences set forth in SEQ ID NOs: 652-654 or 698. In some cases, the nucleic acid molecule encodes an amino acid sequence that is at least 75%, 80%, 85%, 90%, 95%, 98%, or 99%, or 100% identical to any of the sequences set forth in SEQ ID NOs: 655-657 or 699.

Disclosed herein, in some aspects, are methods, compositions, kits, vectors relating to any of the single cytokine-anchor materials, the two cytokine-anchor materials, and the three cytokine-anchor materials (hereinafter collectively “cytokine-anchor materials” unless otherwise noted) disclosed herein.

Disclosed herein, in some aspects, is a nucleic acid molecule comprising a nucleic acid sequence that is at least 75%, 80%, 85%, 90%, 95%, 98%, or 99%, or 100% identical to any of the sequences set forth in SEQ ID NOs: 510-514, 518-519, 521-522, 527-529, 533, 600-602, 606, 611-617, 625-638, 652-654, 658-666, 676-686, or 698. In some cases, the nucleic acid molecule comprises a nucleic acid sequence that is at least 80% identical to any of the sequences set forth in SEQ ID NOs: 510-514, 518-519, 521-522, 527-529, 533, 600-602, 606, 611-617, 625-638, 652-654, 658-666, 676-686, or 698. In some cases, the nucleic acid molecule comprises a nucleic acid sequence that is at least 85% identical to any of the sequences set forth in SEQ ID NOs: 510-514, 518-519, 521-522, 527-529, 533, 600-602, 606, 611-617, 625-638, 652-654, 658-666, 676-686, or 698. In some cases, the nucleic acid molecule comprises a nucleic acid sequence that is at least 90% identical to any of the sequences set forth in SEQ ID NOs: 510-514, 518-519, 521-522, 527-529, 533, 600-602, 606, 611-617, 625-638, 652-654, 658-666, 676-686, or 698. In some cases, the nucleic acid molecule comprises a nucleic acid sequence that is at least 95% identical to any of the sequences set forth in SEQ ID NOs: 510-514, 518-519, 521-522, 527-529, 533, 600-602, 606, 611-617, 625-638, 652-654, 658-666, 676-686, or 698. In some cases, the nucleic acid molecule comprises a nucleic acid sequence that is 100% identical to any of the sequences set forth in SEQ ID NOs: 510-514, 518-519, 521-522, 527-529, 533, 600-602, 606, 611-617, 625-638, 652-654, 658-666, 676-686, or 698.

Disclosed herein, in some aspects, is a polypeptide comprising an amino acid sequence that is at least 75%, 80%, 85%, 90%, 95%, 98%, or 99%, or 100% identical to any of the sequences set forth in SEQ ID NOs: 515-517, 523-526, 530-532, 534, 603-605, 607, 618-624, 639-651, 655-657, 667-675, 687-697, or 699. In some cases, the polypeptide comprises an amino acid sequence that is at least 80% identical to any of the sequences set forth in SEQ ID NOs: 515-517, 523-526, 530-532, 534, 603-605, 607, 618-624, 639-651, 655-657, 667-675, 687-697, or 699. In some cases, the polypeptide comprises an amino acid sequence that is at least 85% identical to any of the sequences set forth in SEQ ID NOs: 515-517, 523-526, 530-532, 534, 603-605, 607, 618-624, 639-651, 655-657, 667-675, 687-697, or 699. In some cases, the polypeptide comprises an amino acid sequence that is at least 90% identical to any of the sequences set forth in SEQ ID NOs: 515-517, 523-526, 530-532, 534, 603-605, 607, 618-624, 639-651, 655-657, 667-675, 687-697, or 699. In some cases, the polypeptide comprises an amino acid sequence that is at least 95% identical to any of the sequences set forth in SEQ ID NOs: 515-517, 523-526, 530-532, 534, 603-605, 607, 618-624, 639-651, 655-657, 667-675, 687-697, or 699. In some cases, the polypeptide comprises an amino acid sequence that is 100% identical to any of the sequences set forth in SEQ ID NOs: 515-517, 523-526, 530-532, 534, 603-605, 607, 618-624, 639-651, 655-657, 667-675, 687-697, or 699.

In some cases, expressing a specific cytokine or combinations of cytokines on the cell membrane includes introducing exogenous nucleic acid molecules for expressing the specific cytokine or combinations thereof and anchoring structures, so that the specific cytokines or combinations thereof are expressed at a level higher than their naturally existing expression level in the host cell or host organism. In other cases, introducing a specific cytokine or combinations of cytokines and a tumor-targeting moiety (e.g., CAR) into T cells and expressing them on the cell membrane can include introducing a nucleic acid molecule comprising a sequence encoding the specific cytokine or combinations of cytokines and the tumor-targeting moiety (e.g., CAR) into immune cells including T cells, NK cells, monocytes, macrophages, dendritic cells, etc. In some cases, the exogenous nucleic acid sequence encodes IL-12p40. In some cases, the exogenous nucleic acid sequence does not encode both IL-12p40 and IL-12p35. In some cases, the exogenous nucleic acid sequence does not encode a stimulus response element (SRE).

Without wishing to be bound by a certain theory, the cytokine-anchor materials disclosed herein can provide engineered immune cells with higher anti-tumor activity compared with engineered immune cells (such as CAR-T cells, etc.) expressing CAR alone (not expressing exogenously introduced cytokines or a combination of cytokines). Cytokine-anchor materials disclosed herein can achieve better therapeutic effect when provided in immunotherapy. Cytokine-anchor materials disclosed herein can allow expression of cytokines or cytokine combinations on cell membrane to regulate the functions of immune cells via a vector. When the vector of the present disclosure is introduced into immune cells, the resulting immune cells can have improved immunotherapeutic effect, including improved proliferation and survival than existing immune cells. By introducing one cytokine, such as an interleukin, or a specific combination of cytokines in immune cells, and expressing the cytokine or specific combinations of cytokines on the cell membrane together with a tumor-targeting moiety, such as a CAR, antitumor activity can be enhanced compared to when the tumor-targeting moiety (e.g., CAR) was expressed alone. In particular, the persistence and proliferation of engineered immune cells can be significantly improved. Delivering engineered immune cells of the cytokine-anchor materials to patients with cancer can produce better therapeutic outcome. In addition, the engineered immune cells of the present disclosure can provide various benefits such as reduced number of administered cells given their enhanced anti-tumor activity without increasing the release of free cytokines, thereby reducing side effects such as cytokine release syndrome (CRS).

Without wishing to be bound by a certain theory, anchoring a specific cytokine or specific combinations of cytokines on surface of an immune cell can retain the activity of the cytokines and can stimulate immune cells precisely in a targeted manner without triggering secretion of additional cytokines that can cause severe CRS. The cytokines that are anchored on the surface of immune cells according to some embodiments of the present disclosure can include different interleukins, such as IL-2, IL-4, IL-7, IL-9, IL-10, IL-12, IL-12p40, IL-15, IL-18, IL-21, IL-23, IL-27, IL-36γ, IL-23p19, or IL-1α or a variant thereof. Such cytokines anchored on the cell membrane can improve the immune cell's ability to proliferate and survive.

Without wishing to be bound by a certain theory, a combination of different cytokines can be anchored on the surface of immune cells, including a combination of different interleukins, such as IL-2, IL-4, IL-7, IL-9, IL-10, IL-12, IL-12p40, IL-15, IL-18, IL-21, IL-23, IL-27, IL-36γ, IL-23p19, or IL-1α or a variant thereof. Such combination of cytokines anchored on the cell membrane can further improve the immune cell's ability to proliferate and survive.

It is to be understood that one, some, or all of the properties of the various embodiments described herein may be applied to any aspect unless the content clearly dictates otherwise. Furthermore, that the various embodiments may be combined to form other embodiments of the present disclosure. These and other aspects of the present disclosure will become apparent to one of skill in the art. These and other embodiments of the present disclosure are further described by the detailed description that follows.

### Definitions

In the present disclosure, wherever aspects are described herein with the language “comprising.” otherwise analogous aspects described in terms of “consisting of” and/or “consisting essentially of” are also provided. All definitions herein described whether specifically mentioned or not, should be construed to refer to definitions as used throughout the specification and attached claims.

Throughout the specification and attached claims, the singular form “a”, “an,” and “the” include plural references unless the context clearly dictates otherwise. For example, the term “a cell” includes a plurality of cells, including mixtures thereof.

In the present disclosure, one, some, or all of the properties of the various embodiments described herein may be applied to any aspect unless the content clearly dictates otherwise. Furthermore, that the various embodiments may be combined to form other embodiments of the present disclosure. These and other aspects of the disclosure will become apparent to one of skill in the art. These and other embodiments of the disclosure are further described by the detailed description herein.

Throughout the specification and attached claims, and unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show. 2nd ed., 2002, CRC Press: The Dictionary of Cell and Molecular Biology, 3rd ed., 1999, Academic Press; and the Oxford Dictionary Of Biochemistry And Molecular Biology, Revised, 2000, Oxford University Press, provide one of ordinary skill with a general dictionary of many of the terms used in this disclosure.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The numbering of amino acids in the variable domain. CDRs and framework regions (FRs), of an antibody follow, unless otherwise indicated, the Kabat definition as set forth in Kabat et al. Sequences of Proteins of Immunological Interest, 5th Ed. Public Health Service, National Institutes of Health, Bethesda, MD. (1991).

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “polypeptide”, “oligopeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides as described herein are based upon an antibody, the polypeptides can occur as single chains or associated chains.

The term “amino acid” refers to natural, unnatural, and synthetic amino acids, including both the D or L optical isomers, and amino acid analogs and peptidomimetics. Standard single or three letter codes are used to designate amino acids.

A “variant” when applied to a protein is a protein with sequence homology to the native biologically active protein that retains at least a portion of the therapeutic and/or biological activity of the biologically active protein. For example, a variant protein may share at least 70%, 75%, 80%, 85%, 90%, 95%, 96%, 97%, 98% or 99% amino acid sequence identity compared with the reference biologically active protein or any ranges in between the at least 70% and 99%. A “variant” as used herein can refer to a cytokine, having substantial or significant sequence identity or similarity to a native cytokine, such as a native cytokine described herein (e.g., IL-2, IL-4, etc.), and retaining one or more biological activities of the native cytokine described herein (e.g., IL-2, IL-4, etc.) or an isoform of the native cytokine described herein. Variants can include, for example, an alteration, substitution, deletion, addition, or chemical modification of one or more amino acids, one or more unnatural amino acids, or any combination thereof of a parent peptide, such as a cytokine described herein (e.g., IL-2, IL-4, etc.), and can still retain the ability to specifically bind to the respective receptor, activate the downstream targets, and/or induce one or more of the differentiation, proliferation (or death) and activity of cells, e.g., T cells and NK cells, to a similar extent, the same extent, or to a higher extent, as the parent peptide. In some cases, the variant is a cytokine. In reference to a parent cytokine, a variant can be at least about 80%, about 90%, about 95%, about 99% or more identical in amino acid sequence to the parent cytokine.

In the context of polypeptides, a “linear sequence” or a “sequence” is an order of amino acids in a polypeptide in an amino to carboxyl terminus direction in which residues that neighbor each other in the sequence are contiguous in the primary structure of the polypeptide. A “partial sequence” is a linear sequence of part of a polypeptide that is known to comprise additional residues in one or both directions.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoramidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2-O-methyl-, 2′-O-allyl, 2-fluoro- or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include embodiments wherein phosphate is replaced by P(O)S (“thioate”), P(S)S (“dithioate”), (O)NR₂ (“amidate”), P(O)R, P(O)OR″, CO or CH₂ (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

A “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen-binding site of antibodies. There are at least two techniques for determining CDRs: (1) an approach based on cross-species sequence variability (i.e., Kabat et al. Sequences of Proteins of Immunological Interest. (5th ed., 1991, National Institutes of Health, Bethesda MD)); and (2) an approach based on crystallographic studies of antigen-antibody complexes (Al-lazikani et al (1997) J. Molec. Biol. 273:927-948)). As used herein, a CDR may refer to CDRs defined by either approach or by a combination of both approaches.

A “constant region” of an antibody refers to the constant region of the antibody light chain or the constant region of the antibody heavy chain, either alone or in combination.

A “host cell” includes an individual cell or cell culture that can be or has been a recipient for vector(s) comprising exogenous polynucleotides. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of the present disclosure.

An “individual” or a “subject” is a mammal, more preferably a human. Mammals also include farm animals, sport animals, pets, primates, horses, dogs, cats, mice and rats.

As used herein. “vector” means a construct, which is capable of delivering, and preferably expressing, one or more gene(s) or sequence(s) of interest in a host cell. Examples of vectors include viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term “effective amount” also applies to a dose that will provide an image for detection by an appropriate imaging method. The specific dose may vary depending on one or more of: the particular agent chosen, the dosing regimen to be followed, whether it is administered in combination with other compounds, timing of administration, the tissue to be imaged, and the physical delivery system in which it is carried. An effective amount of an active agent may be administered in a single dose or in multiple doses.

As used herein. “pharmaceutically acceptable carrier” or “pharmaceutical acceptable excipient” includes any material which, when combined with an active ingredient, allows the ingredient to retain biological activity and is non-reactive with the subject's immune system. Examples include any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well-known conventional methods (see, for example, Remington's Pharmaceutical Sciences, 18th edition, A. Gennaro, ed., Mack Publishing Co., Easton, PA, 1990; and Remington. The Science and Practice of Pharmacy 20th Ed. Mack Publishing. 2000).

Throughout the specification and attached claims, the methods and systems of this disclosure as described herein may employ, unless otherwise indicated, conventional techniques and descriptions of molecular biology (including recombinant techniques), cell biology, biochemistry, microarray and sequencing technology, which are within the skill of those who practice in the art. Such conventional techniques include polymer array synthesis, hybridization and ligation of oligonucleotides, sequencing of oligonucleotides, and detection of hybridization using a label. Specific illustrations of suitable techniques can be had by reference to the examples herein. However, equivalent conventional procedures can, of course, also be used. Such conventional techniques and descriptions can be found in standard laboratory manuals such as Green, et al., Eds., Genome Analysis: A Laboratory Manual Series (Vols. I-IV) (1999); Weiner, et al., Eds., Genetic Variation: A Laboratory Manual (2007); Dieffenbach, Dveksler. Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Green, Molecular Cloning: A Laboratory Manual, 4th Edition (2012) (all from Cold Spring Harbor Laboratory Press); Stryer. L., Biochemistry (4th Ed.) W. H. Freeman, N.Y. (1995); Gait, “Oligonucleotide Synthesis: A Practical Approach” IRL Press, London (1984); Nelson and Cox, Lehninger, Principles of Biochemistry, 6^{th} Ed., W. H. Freeman Pub., New York (2012); R. I. Freshney, Culture of Animal Cells: A Manual of Basic Technique and Specialized Applications, 6^{th} Ed., Wiley-Blackwell (2010); and Berg et al., Biochemistry, 5^{th} Ed., W. H. Freeman Pub., New York (2002), all of which are herein incorporated by reference in their entirety for all purposes. Before the present compositions, research tools and systems and methods are described, it is to be understood that this disclosure is not limited to the specific systems and methods, compositions, targets and uses described, as such may, of course, vary. It is also to be understood that the terminology used herein is for the purpose of describing particular aspects only and is not intended to limit the scope of the present disclosure, which will be limited only by appended claims.

The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain of a stimulatory molecule and/or costimulatory molecule. In some cases, the set of polypeptides are contiguous with each other, e.g., are in the same polypeptide chain, e.g., comprise a chimeric fusion protein. In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen binding domain to an intracellular signaling domain. In some cases, the stimulatory molecule is the zeta chain associated with the T cell receptor complex. In some cases, the cytoplasmic signaling domain further comprises one or more functional signaling domains of at least one costimulatory molecule as defined below. In some cases, the costimulatory molecule is chosen from the costimulatory molecules described herein, e.g., 4-1BB (i.e., CD137), CD27 and/or CD28. In some cases, the CAR comprises a chimeric fusion protein comprising an extracellular antigen binding domain, a transmembrane domain and an intracellular signaling domain.

“Cytokine release syndrome” and “CRS” refer to an acute systemic inflammatory syndrome characterized by fever and multiple organ dysfunction that is associated with chimeric antigen receptor (CAR)-T cell therapy, therapeutic antibodies, and haploidentical allogeneic transplantation, e.g., as described in Frey N. Porter D. Cytokine Release Syndrome with Chimeric Antigen Receptor T Cell Therapy, Biol Blood Marrow Transplant 2019; 25: e123.

In the present disclosure, an “antibody” refers to an immunoglobulin molecule capable of specific binding to a target, such as a carbohydrate, polynucleotide, lipid, polypeptide, etc., through at least one antigen recognition site, located in the variable region of the immunoglobulin molecule. The term as used herein, includes an immunoglobulin molecule that specifically binds to an antigen and comprises an FcR binding site which may or may not be functional. As used in the disclosure, the term encompasses not only intact polyclonal or monoclonal antibodies, but also fragments thereof (such as Fab, Fab′, F(ab′)2, diabodies) Fv fragments and single chain (ScFv) mutants that contain an antigen recognition site or antigen binding site and have ability to bind to an antigen. Antigen-binding antibody or immunoglobulin fragments are well known in the art; such fragment can have a functional or non-functional Fc receptor binding site. Further as used herein, the term is not limited only to intact polyclonal or monoclonal antibodies, multispecific antibodies such as bispecific, or polyspecific antibodies generated from at least two intact antibodies, chimeric antibodies, humanized antibodies, single-chain, chimeric, synthetic, recombinant, hybrid, mutated, grafted antibodies, human antibodies, and any other modified immunoglobulin molecule comprising an antigen binding site so long as the antibodies exhibit the desired biological activity.

An antibody or a CAR that “specifically binds” to an epitope is a term well understood in the art, and methods to determine such specific binding are also well known in the art. A molecule is said to exhibit “specific binding” if it reacts or associates more frequently, more rapidly, with greater duration and/or with greater affinity with a particular cell, protein or substance than it does with alternative cells, proteins or substances. An antibody “specifically binds” or “preferentially binds” to a target if it binds with greater affinity, avidity, more readily, and/or with greater duration than it binds to other substances. For example, an antibody that specifically or preferentially binds to CD19 is an antibody that binds this epitope with greater affinity, avidity, more readily, and/or with greater duration than it binds to other epitopes. As a further example, an antibody (or another moiety) that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding.

A “fragment” when applied to a protein or polypeptide, is a truncated form of a native biologically active protein or polypeptide that may or may not retain at least a portion of the therapeutic and/or biological activity.

“TeIL,” “tethered interleukin,” “membrane-anchored cytokine,” “membrane-bound cytokine,” or “membrane-bound IL” can be used interchangeably to refer to a specific cytokine-anchoring structure as disclosed herein.

The term “sIL” refers to a secreted interleukin or cytokine that is not membrane-anchored or membrane-bound.

### Sequence Identity

The sequence identity with respect to the cytokine-anchor materials or any other amino acid sequences or nucleic acid sequences identified herein, is defined as the percentage of amino acid residues (or nucleotides) in a query sequence that are identical with the amino acid residues of a second, reference polypeptide sequence or a portion thereof (or the nucleotides of a second, reference nucleic acid sequence or a portion thereof), after aligning the sequences and introducing gaps, if necessary, to achieve the maximum percent sequence identity, and not considering any conservative substitutions as part of the sequence identity. Alignment for purposes of determining percent amino acid sequence identity or nucleic acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Percent identity may be measured over the length of an entire defined polypeptide sequence or nucleic acid sequence, or may be measured over a shorter length, for example, over the length of a fragment taken from a larger, defined polypeptide sequence, or larger, defined nucleic acid sequence, for instance, a fragment of at least 15, at least 20, at least 30, at least 40, at least 50, at least 70 or at least 150 contiguous residues or base pairs or nucleotides. Such lengths are exemplary only, and it is understood that any fragment length supported by the sequences shown herein, in the tables, figures or Sequence Listing, may be used to describe a length over which percentage identity may be measured. In some embodiments, percent identity is determined with respect to the full length of a noted reference sequence, such as a sequence provided herein. For example, sequence comparison between two amino acid sequences (or a shorter length thereof) of the present disclosure may be carried out by computer program Blastp (protein-protein BLAST) provided online by Nation Center for Biotechnology Information (NCBI). The percentage amino acid sequence identity of a given amino acid sequence A to a given amino acid sequence B (which can alternatively be phrased as a given amino acid sequence A that has a certain % amino acid sequence identity to a given amino acid sequence B) is calculated by the formula as follows:

$$\frac{X}{Y} \times 100 \%$$

where X is the number of amino acid residues scored as identical matches by the sequence alignment program BLAST in that program's alignment of A and B, and where Y is the total number of amino acid residues in A or B, whichever is shorter.

Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity.

### Signal Peptide

In some aspects, provided herein are signal peptides that are present as part of the polypeptides described in the present disclosure, including the polypeptide in the single cytokine-anchor materials, the first polypeptide and the second polypeptide in the two cytokine-anchor materials, and the first polypeptide, the second polypeptide, and the third polypeptide in the three cytokine-anchor materials. In some cases, the signal peptide is removed from the polypeptide to form the protein in the single cytokine-anchor materials, removed from the first polypeptide and the second polypeptide to form the first protein and the second protein, respectively, in the two cytokine-anchor materials, and removed from the first polypeptide, the second polypeptide, and the third polypeptide to form the first protein, the second protein, and the third protein, respectively, in the three cytokine-anchor materials, during protein processing.

The signal peptide can be encoded by a signal peptide nucleic acid sequence. The signal peptide nucleic acid sequence can be present as part of the nucleic acid sequence described in the present disclosure, including the nucleic acid sequence in the single cytokine-anchor materials, the first nucleic acid sequence and the second nucleic acid sequence in the two cytokine-anchor materials, and the first nucleic acid sequence, the second nucleic acid sequence, and the third nucleic acid sequence in the three cytokine-anchor materials.

Signal peptide can be any suitable peptide that can direct the polypeptide described in the present disclosure to a particular compartment a cell that comprises such polypeptide, such as a membrane of the cell. In some embodiments, signal peptides include peptide that direct intracellular delivery and localization of the peptide and any linked polypeptides to a certain organelle (such as the endoplasmic reticulum) and/or the cell surface. The term “signal peptide” or “signal peptide sequence” as used herein refers to a peptide sequence that can be present at the N-terminus of a newly synthesized polypeptide to be secreted or a newly synthesized transmembrane polypeptide. A signal peptide can direct the polypeptide move through or into the cell membrane of the cell. A signal can be subsequently removed. Specifically, a signal peptide can direct the polypeptide into the secretory pathway of the cell.

In some cases, a signal peptide can be a peptide of any secreted or transmembrane protein that directs the delivery of the polypeptides disclosed herein to the cell membrane and cell surface and provides for the correct localization of the polypeptides of the present disclosure. In some cases, the signal peptide directs the polypeptide of the present disclosure to the cell membrane, where the extracellular portion of the polypeptide is displayed on the cell surface. In some cases, the transmembrane portion spans the plasma membrane, and the active domain is located in the cytoplasmic portion or in the interior of the cell. In some embodiments, the signal peptide is cleaved after passage through the endoplasmic reticulum, referred to as a cleavable signal peptide. In some cases, at the end of the signal peptide, there can be an amino acid that is recognized and cleaved by a signal peptidase. The signal peptidase can cleave during or after translocation, yielding a free signal peptide and mature protein. The free signal peptide can then be digested by specific proteases.

In some embodiments, the signal peptide is a type I, II, III or IV transmembrane protein. In some embodiments, the signal peptide comprises an immunoglobulin heavy chain signal peptide.

In some cases, when another component of the cytokine-anchor materials, such as the cytokine and/or CAR, is expressed within a cell (e.g., an engineered immune cell), the signal peptide expressed together with said component in the same polypeptide directs the nascent protein to the endoplasmic reticulum and subsequently to the cell surface, so that said component of the cytokine-anchor materials is expressed on the cell surface. In some cases, the core of the signal peptide consists of a long stretch of hydrophobic amino acids with a tendency to form a α-helix. In some cases, signal peptides begin with a short stretch of positively charged amino acids, which helps enforce the correct topology of the polypeptide during translocation. The signal peptide can be at a N-terminal to of the polypeptide. In some embodiments, when a cell expresses the membrane-anchored cytokine and/or CAR, the signal sequence can be excised from the membrane-anchored cytokine and/or CAR.

Without wishing to be bound by a certain theory, the signal peptide can facilitate expression of membrane-anchored cytokines and/or CARs and the presence of signal peptides in expressed membrane-anchored cytokines and/or CARs can contribute to membrane anchoring of the cytokines and/or CARs, however, the presence of signal peptides can be irrelevant to the function of cytokines and/or CARs anchored to the cell membrane.

In some embodiments, the signal peptide is a signal peptide from a transmembrane protein. In some embodiments, the signal peptide comprises CD4 signal peptide, CD8α signal peptide, CD28 signal peptide, CD33 signal peptide, CD137 (4-1BB) signal peptide, IL-2 signal peptide, IgE signal peptide. IgG1 signal peptide. GM-CSF signal peptide, HLA-A signal peptide, HLA signal peptide, TCR signal peptide, or β2M signal peptide, or a combination thereof. In some embodiments, the signal peptide comprises CD4 signal peptide, CD8α signal peptide, CD28 signal peptide, CD33 signal peptide, CD137 (4-1BB) signal peptide, IL-2 signal peptide, IgE signal peptide, IgG1 signal peptide. GM-CSF signal peptide, HLA-A signal peptide, HLA signal peptide, TCR signal peptide, or β2M signal peptide, or a variant thereof. In some embodiments, the signal peptide comprises CD4 signal peptide or a variant thereof. In some embodiments, the signal peptide comprises CD8α signal peptide or a variant thereof. In some embodiments, the signal peptide comprises CD28 signal peptide or a variant thereof. In some embodiments, the signal peptide comprises CD33 signal peptide or a variant thereof. In some embodiments, the signal peptide comprises CD137 signal peptide or a variant thereof. In some embodiments, the signal peptide comprises IL-2 signal peptide or a variant thereof. In some embodiments, the signal peptide comprises IgE signal peptide or a variant thereof. In some embodiments, the signal peptide comprises IgG1 signal peptide or a variant thereof. In some embodiments, the signal peptide comprises CD4 signal peptide or a variant thereof. In some embodiments, the signal peptide comprises GM-CSF signal peptide or a variant thereof. In some embodiments, the signal peptide comprises CD4 signal peptide or a variant thereof. In some embodiments, the signal peptide comprises HLA-A signal peptide or a variant thereof. In some embodiments, the signal peptide comprises HLA signal peptide or a variant thereof. In some embodiments, the signal peptide comprises TCR signal peptide or a variant thereof. In some embodiments, the signal peptide comprises β2M signal peptide or a variant thereof.

In some embodiments, the signal peptide is a naturally occurring signal peptide of a wild-type cytokine. In some embodiments, the signal peptide comprises an IL-2 signal peptide, an IL-4 signal peptide, an IL-7 signal peptide, an IL-9 signal peptide, an IL-10 signal peptide, an IL-12p40 signal peptide, an IL-15 signal peptide, an IL-18 signal peptide, an IL-21 signal peptide, an IL-23 signal peptide, an IL-27 signal peptide, an IL-36γ signal peptide, an IL-23p19 signal peptide, or an IL-1α signal peptide, or a functional variant thereof. In some embodiments, the signal peptide comprises IL-4 signal peptide or a variant thereof. In some embodiments, the signal peptide comprises IL-10 signal peptide or a variant thereof. In some embodiments, the signal peptide comprises IL-7 signal peptide or a variant thereof. In some embodiments, the signal peptide comprises IL-9 signal peptide or a variant thereof. In some embodiments, the signal peptide comprises IL-12p40 signal peptide or a variant thereof. In some embodiments, the signal peptide comprises IL-15 signal peptide or a variant thereof. In some embodiments, the signal peptide comprises IL-18 signal peptide or a variant thereof. In some embodiments, the signal peptide comprises IL-21 signal peptide or a variant thereof. In some embodiments, the signal peptide comprises IL-23 signal peptide or a variant thereof. In some embodiments, the signal peptide comprises IL-27 signal peptide or a variant thereof. In some embodiments, the signal peptide comprises IL-36γ signal peptide or a variant thereof. In some embodiments, the signal peptide comprises IL-23p19 signal peptide or a variant thereof. In some embodiments, the signal peptide comprises IL-1α signal peptide or a variant thereof.

In some embodiments, the signal peptide and the cytokine peptide are encoded by the same gene. In some embodiments, the signal peptide and the cytokine peptide are encoded by different genes. In some embodiments, the signal peptide comprises a naturally occurring signal peptide of IL-4 and the cytokine peptide comprises at least a portion of IL-4 or a variant thereof. In some embodiments, the signal peptide comprises a naturally occurring signal peptide of IL-10 and the cytokine peptide comprises at least a portion of IL-10 or a variant thereof. In some embodiments, the signal peptide comprises a naturally occurring signal peptide of IL-4 and the cytokine peptide comprises at least a portion of IL-10 or a variant thereof. In some embodiments, the signal peptide comprises a naturally occurring signal peptide of IL-10 and the cytokine peptide comprises at least a portion of IL-4 or a variant thereof.

In some embodiments, the signal peptide comprises an amino acid sequence that is at least 75%, 80%, 85%, 90%, 95%, 98%, or 99%, or 100% identical to any of the sequences set forth in SEQ ID NOs: 2-7 or 113-114. In some embodiments, the signal peptide comprises an amino acid sequence that is at least 75%, 80%, 85%, 90%, 95%, 98%, or 99%, or 100% identical to the sequence set forth in SEQ ID NO: 2. In some embodiments, the signal peptide comprises an amino acid sequence that is at least 75%, 80%, 85%, 90%, 95%, 98%, or 99%, or 100% identical to the sequence set forth in SEQ ID NO: 3. In some embodiments, the signal peptide comprises an amino acid sequence that is at least 75%, 80%, 85%, 90%, 95%, 98%, or 99%, or 100% identical to the sequence set forth in SEQ ID NO: 4. In some embodiments, the signal peptide comprises an amino acid sequence that is at least 75%, 80%, 85%, 90%, 95%, 98%, or 99%, or 100% identical to the sequence set forth in SEQ ID NO: 5. In some embodiments, the signal peptide comprises an amino acid sequence that is at least 75%, 80%, 85%, 90%, 95%, 98%, or 99%, or 100% identical to the sequence set forth in SEQ ID NO: 6. In some embodiments, the signal peptide comprises an amino acid sequence that is at least 75%, 80%, 85%, 90%, 95%, 98%, or 99%, or 100% identical to the sequence set forth in SEQ ID NO: 7. In some embodiments, the signal peptide comprises an amino acid sequence that is at least 75%, 80%, 85%, 90%, 95%, 98%, or 99%, or 100% identical to the sequence set forth in SEQ ID NO: 113. In some embodiments, the signal peptide comprises an amino acid sequence that is at least 75%, 80%, 85%, 90%, 95%, 98%, or 99%, or 100% identical to the sequence set forth in SEQ ID NO: 114.

In some embodiments, the signal peptide is encoded by the signal peptide nucleic acid sequence that is at least 75%, 80%, 85%, 90%, 95%, 98%, or 99%, or 100% identical to any of the sequences set forth in SEQ ID NOs: 52-59. In some embodiments, the signal peptide is encoded by the signal peptide nucleic acid sequence that is at least 75%, 80%, 85%, 90%, 95%, 98%, or 99%, or 100% identical to the sequence set forth in SEQ ID NO: 52. In some embodiments, the signal peptide is encoded by the signal peptide nucleic acid sequence that is at least 75%, 80%, 85%, 90%, 95%, 98%, or 99%, or 100% identical to the sequence set forth in SEQ ID NO: 53. In some embodiments, the signal peptide is encoded by the signal peptide nucleic acid sequence that is at least 75%, 80%, 85%, 90%, 95%, 98%, or 99%, or 100% identical to the sequence set forth in SEQ ID NO: 54. In some embodiments, the signal peptide is encoded by the signal peptide nucleic acid sequence that is at least 75%, 80%, 85%, 90%, 95%, 98%, or 99%, or 100% identical to the sequence set forth in SEQ ID NO: 55. In some embodiments, the signal peptide is encoded by the signal peptide nucleic acid sequence that is at least 75%, 80%, 85%, 90%, 95%, 98%, or 99%, or 100% identical to the sequence set forth in SEQ ID NO: 56. In some embodiments, the signal peptide is encoded by the signal peptide nucleic acid sequence that is at least 75%, 80%, 85%, 90%, 95%, 98%, or 99%, or 100% identical to the sequence set forth in SEQ ID NO: 57. In some embodiments, the signal peptide is encoded by the signal peptide nucleic acid sequence that is at least 75%, 80%, 85%, 90%, 95%, 98%, or 99%, or 100% identical to the sequence set forth in SEQ ID NO: 58. In some embodiments, the signal peptide is encoded by the signal peptide nucleic acid sequence that is at least 75%, 80%, 85%, 90%, 95%, 98%, or 99%, or 100% identical to the sequence set forth in SEQ ID NO: 59.

TABLE 1A
Exemplary signal peptide amino acid sequences

| SEQ ID | | |
|---|---|---|
| NO: | Amino Acid Sequence | Description |
| 2 | AVMAPRTLLLLLSGALA | Signal (HLA-A) |
| | LTQTWA | |
| 3 | GTSLLCWMALCLLGADHADA | Signal (TCR) |
| 4 | SRSVALAVLALLSLSGLEA | Signal (β2M) |
| 5 | LLAMVLTSALLLCSVAG | Signal (unknown |
| | | source 1) |
| 6 | AAEPVEDNCINFVAMKFID | Signal (unknown |
| | NTLYFIAEDDENLESD | source 2) |
| 7 | RGTPGDADGGGRAVYQ | Signal (unknown |
| | | source 3) |
| 113 | GLTSQLLPPLFFLLACAG | Signal (IL-4) |
| | NFVHG | |
| 114 | HSSALLCCLVLLTGVRA | Signal (IL-10) |

TABLE 1B
Exemplary signal peptide nucleic acid sequences

| SEQ | | |
|---|---|---|
| ID NO: | Nucleic Acid Sequence | Description |
| 52 | gctgtgatggcccctagaaccctgctgctgctgctgagcggcgccc | Signal (HLA-A) |
| | tggccctgacacagacctgggcc | |
| 53 | ggcaccagcctgctgtgctggatggcactgtgcctgctgggagcag | Signal (TCR) |
| | accacgccgatgcc | |
| 54 | tctcgctccgtggccttagctgtgctcgcgctactctctctttctg | Signal (β2M) |
| | gcctggaggct | |
| 55 | ctgctggccatggtgctgacaagcgccctgctgctgtgctctgtgg | Signal (unknown |
| | ccggg | source 1) |
| 56 | gccgctgagcccgtggaggacaactgcatcaacttcgtggccatga | Signal (unknown |
| | agttcatcgacaacaccctgtactttatcgccgaggacgacgagaa | source 2) |
| | cctggagagcgac | |
| 57 | agagggacccccggggacgccgacggaggaggaagagcagtgtacc | Signal (unknown |
| | ag | source 3) |
| 58 | ggtctcacctcccaactgcttccccctctgttcttcctgctagcat | Signal (IL-4) |
| | gtgccggcaactttgtccacgga | |
| 59 | cacagctcagcactgctctgttgcctggtcctcctgactggggtga | Signal (IL-10) |
| | gggcc | |

### Cytokine

In some aspects, provided herein are cytokines or cytokine peptides that are present as part of the polypeptides described in the present disclosure, including the polypeptide in the single cytokine-anchor materials, the first polypeptide and the second polypeptide in the two cytokine-anchor materials, and the first polypeptide, the second polypeptide, and the third polypeptide in the three cytokine-anchor materials. The cytokine or cytokine peptide present as part of the polypeptide is operatively linked to an anchoring structure described herein. The anchoring structure can attach the cytokines to a membrane of a cell.

The cytokines can be encoded by a cytokine nucleic acid sequence. The cytokine nucleic acid sequence can be present as part of the nucleic acid sequences described in the present disclosure, including the nucleic acid sequence in the single cytokine-anchor materials, the first nucleic acid sequence and the second nucleic acid sequence in the two cytokine-anchor materials, and the first nucleic acid sequence, the second nucleic acid sequence, and the third nucleic acid sequence in the three cytokine-anchor materials.

In various embodiments, the term “cytokine” can include full-length cytokines or interleukins or fragments (e.g., truncated forms) or variants thereof, which substantially retain the biological activity of the corresponding wild-type cytokine or interleukin (e.g., having the biological activity that is at least 80%, at least 90%, at least 95%, at least 98%, at least 99%, or at least 100% of the biological activity of the corresponding wild-type cytokine or interleukin. Cytokines can be secreted by immune cells (such as monocytes, macrophages. T cells. B cells, NK cells, etc.). Cytokines can be secreted by certain non-immune cells (such as endothelial cells, epidermal cells, fibroblasts cells, etc.). Cytokines can be produced by monocytes, macrophages, B cells, dendritic cells. TH1 and TH2, mast cells, NK cells and bone-marrow stromal cells. Cytokines can have the ability to regulate multiple cell and body functions, including innate and adaptive immunity, hematopoiesis, cell growth, APSC pluripotent cells, and damaged tissue repair and other functions. Cytokines can include interleukin, interferon-alpha (IFN-alpha), interferon-beta (IFN-beta) or tissue necrosis factor (TNF), etc.

In some embodiments, the cytokines used can be from any mammalian species. In some embodiments, the cytokine is from a species comprising human, equine, bovine, murine, porcine, rabbit, cat, dog, rat, goat, sheep or non-human primate. In some embodiments, the cytokine is from human. In some embodiments, the cytokine may be in a mutated form of its natural or wild-type form.

In some cases, the cytokine is specifically selected from interleukin (interleukin, abbreviated as “IL”). Interleukins are a type of cytokines produced by a variety of cells and act on a variety of cells. Interleukins can play a role in transmitting information, activating and regulating immune cells, mediating T and B cell activation, proliferation and differentiation, and inflammatory responses. Interleukins can be produced by helper CD4+ T lymphocytes. Interleukins can be synthesized by monocytes, macrophages and endothelial cells. Interleukins can bind to receptors and can affect activation and suppression of the immune system and cell division.

In some embodiments, the cytokine comprises an IL from Interleukin-1 family. The Interleukin-1 family can comprise IL-1α, IL-1β, IL-18, IL-33, IL-36α, IL-36β, IL-36γ, IL-1Ra, IL-36Ra and IL-38, and an anti-inflammatory cytokine (IL-37). In some cases, the cytokine comprises a proinflammatory cytokine. A proinflammatory cytokine can comprise IL-2, IL-7, IL-9, IL-12, IL-12p40, IL-15, IL-18, IL-21, IL-23, IL-36γ, IL-23p19, or IL-1α. In some cases, the cytokine comprises an anti-inflammatory cytokine. An anti-inflammatory cytokine can comprise IL-4, IL-10, or IL-27.

In some embodiments, the cytokine comprises an IL from IL-2 family. The IL-2 cytokine family, also known as the common γ-chain family, can comprise IL-2, IL-4, IL-7, IL-9, IL-15 and IL-21, IL-2 family can bind to a common γc receptor, also called CD132. The IL-2 family can act as growth and proliferation factors for progenitors and mature cells.

In some embodiments, the cytokine comprises at least a portion of IL-2, IL-4, IL-7, IL-9, IL-10, IL-12, IL-12p40, IL-15, IL-18, IL-21, IL-23, IL-27, IL-36γ, IL-23p19, or IL-1α or a variant thereof. In some embodiments, the cytokine comprises an amino acid sequence that is at least 75%, 80%, 85%, 90%, 95%, 98%, or 99%, or 100% identical to any of the sequences set forth in SEQ ID NOs: 100-112. In some embodiments, the cytokine is encoded by a nucleic acid sequence that is at least 75%, 80%, 85%, 90%, 95%, 98%, or 99%, or 100% identical to any of the sequences set forth in SEQ ID NOs: 150-162.

In some embodiments, the cytokine comprises an IL-2 or a variant thereof. IL-2 can be mainly produced by CD4+ and CD8+ T cells. IL-2 can also be expressed by dendritic cells and NKs. IL-2 can bind IL-2R, which comprises three subunits (CD25, CD122 and common γc), all necessary for the binding. IL-2 can act in the development of regulatory T (Treg) cells, as a B cell growth factor, stimulates antibody synthesis and promotes proliferation and differentiation of NK cells and T helpers. In some embodiments, the cytokine comprises a human IL-2 or a variant thereof. In some embodiments, the cytokine comprises an amino acid sequence that is at least 75%, 80%, 85%, 90%, 95%, 98%, or 99%, or 100% identical to the sequence set forth in SEQ ID NO: 100. In some embodiments, the cytokine is encoded by a nucleic acid sequence that is at least 75%, 80%, 85%, 90%, 95%, 98%, or 99%, or 100% identical to the sequence set forth in SEQ ID NO: 150.

In some embodiments, the cytokine comprises an IL-4 or a variant thereof. IL-4 can be produced by Th2 cells, basophils, eosinophils and mastocytes. IL-4 can bind to two receptors. IL4-R type I, which comprises CD124 (IL-4 rα) and CD 132, and type II, which comprises IL-4Rα and IL-13Rα1. IL-4 can play several different roles, including regulating allergic conditions and activating the immune response against extracellular parasites. IL-4 can stimulate the development of Th2 cells. In some embodiments, the cytokine comprises a human IL-4 or a variant thereof. In some embodiments, the cytokine comprises an amino acid sequence that is at least 75%, 80%, 85%, 90%, 95%, 98%, or 99%, or 100% identical to the sequence set forth in SEQ ID NO: 101. In some embodiments, the cytokine is encoded by a nucleic acid sequence that is at least 75%, 80%, 85%, 90%, 95%, 98%, or 99%, or 100% identical to the sequence set forth in SEQ ID NO: 151. In some embodiments, the cytokine is encoded by a nucleic acid sequence that is at least 75%, 80%, 85%, 90%, 95%, 98%, or 99%, or 100% identical to the sequence set forth in SEQ ID NO: 163.

In some embodiments, the cytokine comprises an IL-7 or a variant thereof. IL-7 can be a homeostatic cytokine. IL-7 can be found in T cells, progenitors of B cells and bone marrow macrophages. IL-7 can bind to its receptor, IL-7R, which comprises a γ-chain fraction and an IL-7Rα (CD127). IL-7 can be involved in the survival and proliferation of thymocytes and in the development of naïve and memory B and T cells, mature T cells and NKs. In some embodiments, the cytokine comprises a human IL-7 or a variant thereof. In some embodiments, the cytokine comprises an amino acid sequence that is at least 75%, 80%, 85%, 90%, 95%, 98%, or 99%, or 100% identical to the sequence set forth in SEQ ID NO: 102. In some embodiments, the cytokine is encoded by a nucleic acid sequence that is at least 75%, 80%, 85%, 90%, 95%, 98%, or 99%, or 100% identical to the sequence set forth in SEQ ID NO: 152. In some embodiments, the human IL-7 or the variant thereof comprises an amino acid sequence that is at least 75%, 80%, 85%, 90%, 95%, 98%, or 99%, or 100% identical to the sequence set forth in GenBank Accession Nos AAC63047.1, EAW87060.1, EAW87061.1, EAW87062.1, ABK41904.1, BAD89408.1, BAD89409.1, BAD89411.1, BAD89412.1, BAD89414.1, BAD89422.1, BAF84227.1, AAH47698.1, ACX53627.1, AAA59156.1, ANQ68335.1.

In some embodiments, the cytokine comprises an IL-9 or a variant thereof. IL-9 can be produced by Th2 cells, eosinophils, or mastocytes. IL-9 can bind to its receptor, IL-9R, which comprises CD132 and IL-9Rα units. IL-9 can be a potent growth factor for T cells and mastocytes. IL-9 can inhibit cytokine production by Th1 cells. IgE production, and mucus secretion by bronchial epithelium. In some embodiments, the cytokine comprises a human IL-9 or a variant thereof. In some embodiments, the cytokine comprises an amino acid sequence that is at least 75%, 80%, 85%, 90%, 95%, 98%, or 99%, or 100% identical to the sequence set forth in SEQ ID NO: 103. In some embodiments, the cytokine is encoded by a nucleic acid sequence that is at least 75%, 80%, 85%, 90%, 95%, 98%, or 99%, or 100% identical to the sequence set forth in SEQ ID NO: 153.

In some embodiments, the cytokine comprises an IL-10 or a variant thereof. In some embodiments, the cytokine comprises a human IL-10) or a variant thereof. In some embodiments, the cytokine comprises an amino acid sequence that is at least 75%, 80%, 85%, 90%, 95%, 98%, or 99%, or 100% identical to the sequence set forth in SEQ ID NO: 104. In some embodiments, the cytokine is encoded by a nucleic acid sequence that is at least 75%, 80%, 85%, 90%, 95%, 98%, or 99%, or 100% identical to the sequence set forth in SEQ ID NO: 154. In some embodiments, the cytokine is encoded by a nucleic acid sequence that is at least 75%, 80%, 85%, 90%, 95%, 98%, or 99%, or 100% identical to the sequence set forth in SEQ ID NO: 164.

In some embodiments, the cytokine comprises an IL-12 or a variant thereof. IL-12 can comprise IL-12p35 and IL-12p40 subunits. Co-expression of IL-12p35 and IL-12p40 subunits can lead to secretion of a disulfide-linked bioactive IL-12p70, IL-12 can be produced by a variety of hematopoietic cell types, antigen-presenting cells, such as dendritic cells and macrophages. IL-12 can bind to its receptor, IL-12Rβ1/IL-12Rβ2, which can be expressed on activated T cells, NK cells and dendritic cells. Binding of IL-12 to its receptor can activate TYK2 (tyrosine kinase 2), JAK2 and STAT pathways. In some embodiments, the cytokine comprises a human IL-12 or a variant thereof. In some embodiments, the human IL-12 or a variant thereof comprises an amino acid sequence that is at least 75%, 80%, 85%, 90%, 95%, 98%, or 99%, or 100% identical to the sequence set forth in GenBank Accession Nos AAM34792.1, AAG32620.1, AAG32620.1, CCA63965.1, AJQ18452.1, AAD56386.1, AAL05890.1, AAL05891, AAH67502.11, AAH67498.1, AAH67498.1, AAH67500.1, AAH67501.1, AAH74723.1, ABM53138.1, AAA35695.1, AAA59938.1.

In some embodiments, the cytokine comprises an IL-12p40 or a variant thereof. In some embodiments, the cytokine comprises a human IL-12p40 or a variant thereof. In some embodiments, the cytokine comprises an amino acid sequence that is at least 75%, 80%, 85%, 90%, 95%, 98%, or 99%, or 100% identical to the sequence set forth in SEQ ID NO: 105. In some embodiments, the cytokine is encoded by a nucleic acid sequence that is at least 75%, 80%, 85%, 90%, 95%, 98%, or 99%, or 100% identical to the sequence set forth in SEQ ID NO: 155.

In some embodiments, the cytokine comprises an IL-15 or a variant thereof. IL-15 can bind to its receptor, IL-15R, which comprises a CD132 subunit. IL-15Rα, and IL-2Rβ chains. IL-15 can be produced by keratinocytes, skeletal muscle cells, monocytes and activated CD4+ T cells, in response to signals that trigger innate immunity. IL-15 can have similar structure as well as some identical functions to IL-2, such as T cell activation and stimulation of NK cell proliferation. IL-15 can also be involved with CD8+ memory cell. NK cell, and NKT-cell homeostasis. In some embodiments, the cytokine comprises a human IL-15 or a variant thereof. In some embodiments, the cytokine comprises an amino acid sequence that is at least 75%, 80%, 85%, 90%, 95%, 98%, or 99%, or 100% identical to the sequence set forth in SEQ ID NO: 106. In some embodiments, the cytokine is encoded by a nucleic acid sequence that is at least 75%, 80%, 85%, 90%, 95%, 98%, or 99%, or 100% identical to the sequence set forth in SEQ ID NO: 156. In some embodiments, the human IL-15 or a variant thereof comprises an amino acid sequence that is at least 75%, 80%, 85%, 90%, 95%, 98%, or 99%, or 100% identical to the sequence set forth in GenBank Accession Nos: EAX05083.1, EAX05084.1, EAX05085.1, EAX05086.1, EAX05087.1, NP_751915.1, NP_000576.1, CAJ13397.1, CAJ32191.1, CAL40354.1, CAS97649.1, CBI67687.1, AAI00963.1, AAI00964.1, AAI00962.1, AAB97518.1, BAG53839.1, BAF83308.1, AAU21241.1, CAA71044.1, AAH18149.1, AAB97518.1, CAA63914.1, CAG46777.1, CAG46804.1, AAD15004.1, AAA21551.1, and CAA63913.1.

In some embodiments, the cytokine comprises an IL-18 or a variant thereof. IL-18 can promote TH1 and Th2 cells responses. IL-18 can induce IL-13 production in T cells and NK cells together with IL-2, IL-18 can enhance NK toxicity by promoting the expression of Fas ligand in NK cells. IL-18 can be involved in several autoimmune diseases, in myocardial infarction, metabolic syndromes and others. In some embodiments, the cytokine comprises a human IL-18 or a variant thereof. In some embodiments, the cytokine comprises an amino acid sequence that is at least 75%, 80%, 85%, 90%, 95%, 98%, or 99%, or 100% identical to the sequence set forth in SEQ ID NO: 107. In some embodiments, the cytokine is encoded by a nucleic acid sequence that is at least 75%, 80%, 85%, 90%, 95%, 98%, or 99%, or 100% identical to the sequence set forth in SEQ ID NO: 157.

In some embodiments, the cytokine comprises an IL-21 or a variant thereof. IL-21 can be produced by T cells, NKT cells and Th17. IL-21 can bind to its receptor, comprising CD132 and IL-21R. IL-21 can be involved with B cells functions. IL-21 can increase the proliferation of CD8+ T cells, NK cells and NKT. In some embodiments, the cytokine comprises a human IL-21 or a variant thereof. In some embodiments, the cytokine comprises an amino acid sequence that is at least 75%, 80%, 85%, 90%, 95%, 98%, or 99%, or 100% identical to the sequence set forth in SEQ ID NO: 108. In some embodiments, the cytokine is encoded by a nucleic acid sequence that is at least 75%, 80%, 85%, 90%, 95%, 98%, or 99%, or 100% identical to the sequence set forth in SEQ ID NO: 158. In some embodiments, the human IL-15 or a variant thereof comprises an amino acid sequence that is at least 75%, 80%, 85%, 90%, 95%, 98%, or 99%, or 100% identical to the sequence set forth in GenBank Accession Nos: AAU88182.1, EAX05226.1, CA194500.1, CAJ47524.1, CAL81203.1, CAN87399.1, CAS03522.1, CAV33288.1, CBE74752.1, CB170418.1, CBI85469.1, CB185472.1, CBL93962.1, CCA63962.1, AAG29348.1, AAH66258.1, AAH66259.1, AAH66260.1, AAH66261.1, AAH66262.1, AAH69124.1, ABG36529.1, and BBA22643.1.

In some embodiments, the cytokine comprises an IL-27 or a variant thereof. In some embodiments, the cytokine comprises a human IL-27 or a variant thereof. IL-27 can bind to its receptor IL-27R, which comprises a ubiquitously expressed gp130 protein and a WSX-1/TCCR. The biologic effects of IL-27 can be mediated through activation of JAK1, JAK2, TYK2, STAT1 and STAT3. In some embodiments, the cytokine comprises an amino acid sequence that is at least 75%, 80%, 85%, 90%, 95%, 98%, or 99%, or 100% identical to the sequence set forth in SEQ ID NO: 109. In some embodiments, the cytokine is encoded by a nucleic acid sequence that is at least 75%, 80%, 85%, 90%, 95%, 98%, or 99%, or 100% identical to the sequence set forth in SEQ ID NO: 159.

In some embodiments, the cytokine comprises an IL-36γ or a variant thereof. In some embodiments, the cytokine comprises a human IL-36γ or a variant thereof. In some embodiments, the cytokine comprises an amino acid sequence that is at least 75%, 80%, 85%, 90%, 95%, 98%, or 99%, or 100% identical to the sequence set forth in SEQ ID NO: 110. In some embodiments, the cytokine is encoded by a nucleic acid sequence that is at least 75%, 80%, 85%, 90%, 95%, 98%, or 99%, or 100% identical to the sequence set forth in SEQ ID NO: 160.

In some embodiments, the cytokine comprises an IL-23 or a variant thereof. In some embodiments, the cytokine comprises an IL-23p19 or a variant thereof. IL-23 can bind to its receptor which derives from the combination of IL-12Rβ1 with a unique IL-23 receptor subunit (IL-23R). The biologic effects of IL-23 on its target cells can be mediated through activation of TYK2, JAK2, STAT3 and STAT4. In some embodiments, the cytokine comprises a human IL-23 or a variant thereof. In some embodiments, the cytokine comprises a human IL-23p19 or a variant thereof. In some embodiments, the cytokine comprises an amino acid sequence that is at least 75%, 80%, 85%, 90%, 95%, 98%, or 99%, or 100% identical to the sequence set forth in SEQ ID NO: 111. In some embodiments, the cytokine is encoded by a nucleic acid sequence that is at least 75%, 80%, 85%, 90%, 95%, 98%, or 99%, or 100% identical to the sequence set forth in SEQ ID NO: 161.

In some embodiments, the cytokine comprises an IL-1α or a variant thereof. In some embodiments, the cytokine comprises a human IL-1α or a variant thereof. In some embodiments, the cytokine comprises an amino acid sequence that is at least 75%, 80%, 85%, 90%, 95%, 98%, or 99%, or 100% identical to the sequence set forth in SEQ ID NO: 112. In some embodiments, the cytokine is encoded by a nucleic acid sequence that is at least 75%, 80%, 85%, 90%, 95%, 98%, or 99%, or 100% identical to the sequence set forth in SEQ ID NO: 162.

TABLE 2A
Exemplary cytokine peptide amino acid sequences

| SEQ | | |
|---|---|---|
| ID NO: | Amino Acid Sequence | Description |
| 100 | APTSSSTKKTQLQLEHLLLDLQMILNGINNYKNPKLTRMLTFKFYMPKKATE | IL-2 |
| | LKHLQCLEEELKPLEEVLNLAQSKNFHLRPRDLISNINVIVLELKGSETTFM | |
| | CEYADETATIVEFLNRWITFCQSIISTLT | |
| 101 | HKCDITLQEIIKTLNSLTEQKTLCTELTVTDIFAASKNTTEKETFCRAATVL | IL-4 |
| | RQFYSHHEKDTRCLGATAQQFHRHKQLIRFLKRLDRNLWGLAGLNSCPVKEA | |
| | NQSTLENFLERLKTIMREKYSKCSS | |
| 102 | DCDIEGKDGKQYESVLMVSIDQLLDSMKEIGSNCLNNEFNFFKRHICDANKE | IL-7 |
| | GMFLFRAARKLRQFLKMNSTGDFDLHLLKVSEGTTILLNCTGQVKGRKPAAL | |
| | GEAQPTKSLEENKSLKEQKKLNDLCFLKRLLQEIKTCWNKILMGTKEH | |
| 103 | QGCPTLAGILDINFLINKMQEDPASKCHCSANVTSCLCLGIPSDNCTRPCES | IL-9 |
| | ERLSQMTNTTMQTRYPLIFSRVKKSVEVLKNNKCPYFSCEQPCNQTTAGNAL | |
| | TFLKSLLEIFQKEKMRGMRGKI | |
| 104 | SPGQGTQSENSCTHFPGNLPNMLRDLRDAFSRVKTFFQMKDQLDNLLLKESL | IL-10 |
| | LEDFKGYLGCQALSEMIQFYLEEVMPQAENQDPDIKAHVNSLGENLKTLRLR | |
| | LRRCHRFLPCENKSKAVEQVKNAFNKLQEKGIYKAMSEFDIFINYIEAYMTM | |
| | KIRN | |
| 105 | IWELKKDVYVVELDWYPDAPGEMVVLTCDTPEEDGITWTLDQSSEVLGSGKT | IL-12p40 |
| | LTIQVKEFGDAGQYTCHKGGEVLSHSLLLLHKKEDGIWSTDILKDQKEPKNK | |
| | TFLRCEAKNYSGRFTCWWLTTISTDLTFSVKSSRGSSDPQGVTCGAATLSAE | |
| | RVRGDNKEYEYSVECQEDSACPAAEESLPIEVMVDAVHKLKYENYTSSFFIR | |
| | DIIKPDPPKNLQLKPLKNSRQVEVSWEYPDTWSTPHSYFSLTFCVQVQGKSK | |
| | REKKDRVFTDKTSATVICRKNASISVRAQDRYYSSSWSEWASVPCS | |
| 106 | GIHVFILGCFSAGLPKTEANWVNVISDLKKIEDLIQSMHIDATLYTESDVHP | IL-15 |
| | SCKVTAMKCFLLELQVISLESGDASIHDTVENLIILANNSLSSNGNVTESGC | |
| | KECEELEEKNIKEFLQSFVHIVQMFINTSRA | |
| 107 | YFGKLESKLSVIRNLNDQVLFIDQGNRPLFEDMTDSDCRDNAPRTIFIISMY | IL-18 |
| | KDSQPRGMAVTISVKCEKISTLSCENKIISFKEMNPPDNIKDTKSDIIFFQR | |
| | SVPGHDNKMQFESSSYEGYFLACEKERDLFKLILKKEDELGDRSIMFTVQNE | |
| | D | |
| 108 | QGQDRHMIRMRQLIDIVDQLKNYVNDLVPEFLPAPEDVETNCEWSAFSCFQK | IL-21 |
| | AQLKSANTGNNERIINVSIKKLKRKPPSTNAGRRQKHRLTCPSCDSYEKKPP | |
| | KEFLERFKSLLQKMIHQHLSSRTHGSEDSRA | |
| 109 | FPRPPGRPQLSLQELRREFTVSLHLARKLLSEVRGQAHRFAESHLPGVNLYL | IL-27 |
| | LPLGEQLPDVSLTFQAWRRLSDPERLCFISTTLQPFHALLGGLGTQGRWTNM | |
| | ERMQLWAMRLDLRDLQRHLRFQVLAAGFNLPEEEEEEEEEEEEERKGLLPGA | |
| | LGSALQGPAQVSWPQLLSTYRLLHSLELVLSRAVRELLLLSKAGHSVWPLGF | |
| | PTLSPQP | |
| 110 | SMCKPITGTINDLNQQVWTLQGQNLVAVPRSDSVTPVTVAVITCKYPEALEQ | IL-36Y |
| | GRGDPIYLGIQNPEMCLYCEKVGEQPTLQLKEQKIMDLYGQPEPVKPFLFYR | |
| | AKTGRTSTLESVAFPDWFIASSKRDQPIILTSELGKSYNTAFELNIND | |
| 111 | RAVPGGSSPAWTQCQQLSQKLCTLAWSAHPLVGHMDLREEGDEETTNDVPHI | IL-23p19 |
| | QCGDGCDPQGLRDNSQFCLQRIHQGLIFYEKLLGSDIFTGEPSLLPDSPVGQ | |
| | LHASLLGLSQLLQPEGHHWETQQIPSLSPSQPWQRLLLRFKILRSLQAFVAV | |
| | AARVFAHGAATLSP | |
| 112 | MAKVPDMFEDLKNCYSENEEDSSSIDHLSLNQKSFYHVSYGPLHEGCMDQSV | IL-la |
| | SLSISETSKTSKLTFKESMVVVATNGKVLKKRRLSLSQSITDDDLEAIANDS | |
| | EEEIIKPRSAPFSFLSNVKYNFMRIIKYEFILNDALNQSIIRANDQYLTAAA | |
| | LHNLDEAVKFDMGAYKSSKDDAKITVILRISKTQLYVTAQDEDQPVLLKEMP | |
| | EIPKTITGSETNLLFFWETHGTKNYFTSVAHPNLFIATKQDYWVCLAGGPPS | |
| | ITDFQILENQA | |

TABLE 2B
Exemplary cytokine nucleic acid sequences

| SEQ | | |
|---|---|---|
| ID NO: | Nucleic Acid Sequence | Description |
| 150 | gcccctaccagcagctccaccaagaagacccagctgcagctggagcacctcc | IL-2 |
| | tgctggacctgcagatgatcctgaacggcatcaacaactataagaatcctaa | |
| | gctgacaagaatgctgacctttaagttctacatgcctaagaaagctaccgag | |
| | ctgaagcacctgcaatgtctggaagaggagctgaaacctctggaagaggtgc | |
| | tgaatctggcccagagcaaaaacttccacctcagacctagagatctgatcag | |
| | caacatcaacgtgatcgtgctggaactgaagggcagcgagacaacattcatg | |
| | tgcgagtacgccgacgagacagccaccattgtggaattcctgaaccggtgga | |
| | tcaccttctgccagtccatcatctctacactgacc | |
| 151 | cacaagtgcgacatcaccctgcaagagatcatcaagaccctgaacagcctga | IL-4 |
| | ccgagcagaagaccctgtgcaccgagctgaccgtgaccgacatcttcgccgc | |
| | tagcaagaacaccaccgagaaggagaccttctgcagagccgccaccgtgctg | |
| | agacagttctacagccaccacgagaaggacacaagatgcctgggcgccaccg | |
| | ctcagcagttccacagacacaagcagctgatcagattcctgaagagactgga | |
| | cagaaacctgtggggcctggccggcctgaacagctgccccgtgaaggaggcc | |
| | aatcagagcaccctggagaacttcctggagagactgaagaccatcatgagag | |
| | agaagtacagcaagtgcagcagc | |
| 152 | gattgtgatattgaaggtaaagatggcaaacaatatgagagtgttctaatgg | IL-7 |
| | tcagcatcgatcaattattggacagcatgaaagaaattggtagcaattgcct | |
| | gaataatgaatttaacttttttaaaagacatatctgtgatgctaataaggaa | |
| | ggtatgtttttattccgtgctgctcgcaagttgaggcaatttcttaaaatga | |
| | atagcactggtgattttgatctccacttattaaaagtttcagaaggcacaac | |
| | aatactgttgaactgcactggccaggttaaaggaagaaaaccagctgccctg | |
| | ggtgaagcccaaccaacaaagagtttggaagaaaataaatctttaaaggaac | |
| | agaaaaaactgaatgacttgtgtttcctaaagagactattacaagagataaa | |
| | aacttgttggaataaaattttgatgggcactaaagaacac | |
| 153 | caggggtgccctaccctggctggaatcctggacatcaacttcctgatcaaca | IL-9 |
| | agatgcaggaggaccccgcttccaagtgccactgctccgccaatgtgacctc | |
| | ctgcctgtgtctgggcattccctccgacaactgtactagaccctgcttcagc | |
| | gagcgcctgagccagatgactaacaccaccatgcagacccgctaccccctga | |
| | tcttctcccgcgtcaagaagtccgtcgaagtgctgaagaacaacaagtgccc | |
| | ctacttctcctgcgagcagccctgcaaccagaccaccgccggcaacgccctg | |
| | accttcctgaaaagcctgctggagatcttccagaaggagaaaatgcgcggca | |
| | tgagaggcaagatc | |
| 154 | agccccgggcaaggcacacagagcgagaacagctgcacccacttccccggca | IL-10 |
| | acctgcccaacatgctgagagacctgagagacgccttcagcagagtgaagac | |
| | cttctttcagatgaaggatcagctggacaacctgctcctgaaggagagcctg | |
| | ctggaggacttcaagggctacctgggctgccaagccctgagcgagatgattc | |
| | agttctacctggaagaggtgatgccccaagccgagaaccaagaccccgacat | |
| | caaggcccacgtgaacagcctgggcgagaacctgaagaccctgagactgaga | |
| | ctgagaagatgccacagattcctgccctgcgagaacaagagcaaggccgtgg | |
| | agcaagtgaagaacgccttcaacaagctgcaagagaagggcatctacaaggc | |
| | catgagcgagttcgacatcttcatcaactacatcgaggcctacatgaccatg | |
| | aagatcagaaac | |
| 155 | atatgggaactgaagaaagatgtttatgtcgtagaattggattggtatccgg | IL-12p40 |
| | atgcccctggagaaatggtggtcctcacctgtgacacccctgaagaagatgg | |
| | tatcacctggaccttggaccagagcagtgaggtcttaggctctggcaaaacc | |
| | ctgaccatccaagtcaaagagtttggagatgctggccagtacacctgtcaca | |
| | aaggaggcgaggttctaagccattcgctcctgctgcttcacaaaaaggaaga | |
| | tggaatttggtccactgatattttaaaggaccagaaagaacccaaaaataag | |
| | acctttctaagatgcgaggccaagaattattctggacgtttcacctgctggt | |
| | ggctgacgacaatcagtactgatttgacattcagtgtcaaaagcagcagagg | |
| | ctcttctgacccccaaggggtgacgtgcggagctgctacactctctgcagag | |
| | agagtcagaggggacaacaaggagtatgagtactcagtggagtgccaggagg | |
| | acagtgcctgcccagctgctgaggagagtctgcccattgaggtcatggtgga | |
| | tgccgttcacaagctcaagtatgaaaactacaccagcagcttcttcatcagg | |
| | gacatcatcaaacctgacccacccaagaacttgcagctgaagccattaaaga | |
| | attctcggcaggtggaggtcagctgggagtaccctgacacctggagtactcc | |
| | acattcctacttctccctgacattctgcgttcaggtccagggcaagagcaag | |
| | agagaaaagaaagatagagtcttcacggacaagacctcagccacggtcatct | |
| | gccgcaaaaatgccagcattagcgtgcgggcccaggaccgctactatagctc | |
| | atcttggagcgaatgggcatctgtgccctgcagt | |
| 156 | ggcatccacgtgttcatcctgggctgcttcagcgccggacttcctaaaacag | IL-15 |
| | aggccaactgggtgaacgtgattagcgacctgaagaagatcgaggacctgat | |
| | ccagagcatgcacatcgacgccaccctgtacaccgagagcgatgtgcatcct | |
| | agctgcaaggtgaccgccatgaagtgcttcctgctggagctgcaggtgatca | |
| | gcctggagagcggagatgccagcattcacgacacagtggaaaatctgatcat | |
| | cctggccaacaacagcctgagcagcaacggcaatgtgaccgagagcggctgt | |
| | aaggagtgcgaggaactggaggagaagaacatcaaggagttcctgcagagct | |
| | tcgtgcacatcgtgcagatgttcatcaacaccagcagagcc | |
| 157 | tactttggcaagctggagagcaagctgagcgtgatccggaacctgaacgacc | IL-18 |
| | aggtgctgttcatcgaccagggcaatcggcctctgtttgaggacatgaccga | |
| | cagcgactgcagagacaacgcacccagaaccatcttcatcatctccatgtac | |
| | aaggactcccagccaaggggcatggccgtgaccatcagcgtgaaatgcgaga | |
| | aaatcagcacactgtcatgcgagaacaagatcatcagcttcaaggaaatgaa | |
| | cccccccgacaacatcaaggacacaaaaagcgacatcatcttcttccagaga | |
| | tccgtccccggccacgacaacaaaatgcagttcgagagctcctcctacgagg | |
| | gctacttcctggcctgcgaaaaagaaagagacctgttcaagctgatcctgaa | |
| | gaaggaggacgagctgggcgacagaagcatcatgttcaccgtgcagaacgag | |
| | gac | |
| 158 | cagggccaggacagacacatgatcagaatgagacagctgatcgacatcgtgg | IL-21 |
| | accagctgaagaactacgtgaacgacctggtgcccgagttcctgcccgctcc | |
| | tgaagacgtggaaacaaactgtgagtggagcgctttcagctgcttccagaag | |
| | gcccagctgaagagcgccaataccggaaacaatgagagaatcatcaacgtga | |
| | gcatcaagaagctgaagagaaagccccccagcaccaacgccggaagaagaca | |
| | aaaacatagactgacctgccccagctgcgatagctacgagaaaaagcccccc | |
| | aaggagttcctggagagattcaagagcctgctgcagaagatgatccaccagc | |
| | acctgagcagcagaacccacggctctgaagatagcagagct | |
| 159 | ttccctcggccccccggcagacctcagctgagcctgcaagagctgagaagag | IL-27 |
| | agttcaccgtgagcctgcacctggctagaaagctgctgagcgaggtgagagg | |
| | ccaagcccacagattcgccgagagccacctgcccggcgtgaacctgtacctg | |
| | ctgcccctgggcgagcagctgcccgacgtgagcctgaccttccaagcctgga | |
| | gaagactgagcgaccccgagagactgtgcttcatcagcaccaccctgcagcc | |
| | cttccacgccctgctgggcggcctgggcacccaaggcagatggaccaacatg | |
| | gagagaatgcagctgtgggccatgagactggacctgagagacctgcagagac | |
| | acctgagattccaagtgctggccgccggcttcaacctgcccgaggaagagga | |
| | agaggaagaggaggaagaggaggaagagcggaagggcctgctgcctggcgct | |
| | ctcggcagcgccctgcaagggcctgctcaagtgagctggcctcagctgctga | |
| | gcacctacagactgctgcacagcctggagctggtgctgagcagagccgtgag | |
| | agagctgctcctgctgagcaaggccggccacagcgtgtggcccctgggcttc | |
| | cccaccctgagccctcagccc | |
| 160 | agcatgtgcaagcccatcaccgggaccatcaacgacctgaaccagcaggtgt | IL-36Y |
| | ggaccctgcagggacagaacctggtggccgtgcccagaagcgatagcgtgac | |
| | acccgtgaccgtcgccgtcattacctgcaagtaccccgaagccctggaacag | |
| | ggcagaggcgatcccatctacctggggattcaaaaccccgaaatgtgcctgt | |
| | actgcgagaaggtcggcgagcagcctaccctgcagctgaaggaacagaaaat | |
| | catggacctgtacggacagcctgagcccgtgaaacccttcctgttctacaga | |
| | gctaagaccggccgcaccagcacactggagagcgtcgccttccccgactggt | |
| | tcatcgccagcagcaaaagagaccagcccatcatcctgacctcagagctggg | |
| | aaagagctacaacaccgccttcgagctgaacatcaacgat | |
| 161 | agggctgtccccgggggttcctccccggcctggacccagtgtcagcagcttt | IL-23p19 |
| | cacagaagctgtgcactctggcctggtctgctcaccccctggtgggccatat | |
| | ggatctgcgcgaggagggcgacgaggaaacgaccaacgacgtgccacacatc | |
| | cagtgcggggacggctgtgatcctcagggtctccgtgacaactcccagtttt | |
| | gcctgcagcgcatccaccagggcctcatcttttacgagaaactgctgggctc | |
| | cgacatcttcaccggcgagccctctctgctgcccgacagcccggttggacag | |
| | ctgcacgcctccctactgggtttgagccagctgctccagccagagggccacc | |
| | actgggagacccagcaaattccctctcttagtccttcgcagccgtggcaacg | |
| | cctgcttctccgcttcaagattctgcggtcgttgcaggccttcgtggccgtg | |
| | gcggctcgagtgttcgcgcatggagcagccacactgagccct | |
| 162 | atggccaaggtgcccgacatgttcgaggacctgaagaactgctacagcgaga | IL-la |
| | acgaggaggacagcagcagcatcgaccacctgagcctgaatcagaagagctt | |
| | ctaccacgtgagctacggccccctgcacgagggctgcatggatcagagcgtg | |
| | agcctgagcatcagcgagacaagcaagacaagcaagctgaccttcaaggaga | |
| | gcatggtggtcgtggccaccaacggcaaggtgctgaagaagagaagactgag | |
| | cctgagccaatccattacagacgatgacctggaggccatcgccaacgacagc | |
| | gaggaagagatcatcaagcctagaagcgcccccttcagcttcctgagcaacg | |
| | tgaagtacaacttcatgagaatcatcaagtacgagttcatcctgaacgacgc | |
| | cctgaatcagagcatcatcagagccaacgatcagtacctgaccgccgctgcc | |
| | ctgcacaacctggacgaggccgtgaagttcgacatgggcgcctacaagagca | |
| | gcaaggacgacgccaagatcaccgtgatcctgagaatcagcaagacacagct | |
| | gtacgtgaccgcccaagacgaggatcagcccgtgctgctgaaggagatgccc | |
| | gagatccccaagaccatcaccggcagcgagaccaacctgctgttcttctggg | |
| | agacccacggcaccaagaactacttcacaagcgtggcccaccccaacctgtt | |
| | catcgccaccaagcaagactactgggtgtgcctggccggcggcccccctagc | |
| | atcaccgactttcagatcctggagaaccaagcc | |
| 163 | cacaagtgcgatatcaccttacaggagatcatcaaaactttgaacagcctca | IL-4 |
| | cagagcagaagactctgtgcaccgagttgaccgtaacagacatctttgctgc | |
| | ctccaagaacacaactgagaaggaaaccttctgcagggctgcgactgtgctc | |
| | cggcagttctacagccaccatgagaaggacactcgctgcctgggtgcgactg | |
| | cacagcagttccacaggcacaagcagctgatccgattcctgaaacggctcga | |
| | caggaacctctggggcctggcgggcttgaattcctgtcctgtgaaggaagcc | |
| | aaccagagtacgttggaaaacttcttggaaaggctaaagacgatcatgagag | |
| | agaaatattcaaagtgttcgagc | |
| 164 | agcecaggccagggcacccagtctgagaacagctgcacccacttcccaggca | IL-10 |
| | acctgcctaacatgcttcgagatctccgagatgccttcagcagagtgaagac | |
| | tttctttcaaatgaaggatcagctggacaacttgttgttaaaggagtccttg | |
| | ctggaggactttaagggttacctgggttgccaagccttgtctgagatgatcc | |
| | agttttacctggaggaggtgatgccccaagctgagaaccaagacccagacat | |
| | caaggcgcatgtgaactccctgggggagaacctgaagaccctcaggctgagg | |
| | ctacggcgctgtcatcgatttcttccctgtgaaaacaagagcaaggccgtgg | |
| | agcaggtgaagaatgcctttaataagctccaagagaaaggcatctacaaagc | |
| | catgagtgagtttgacatcttcatcaactacatagaagcctacatgacaatg | |
| | aagatacgaaac | |

### Anchoring Structure

In some aspects, provided herein are anchoring structures that are present as part of the polypeptides described in the present disclosure, including the polypeptide in the single cytokine-anchor materials, the first polypeptide and the second polypeptide in the two cytokine-anchor materials, and the first polypeptide, the second polypeptide, and the third polypeptide in the three cytokine-anchor materials. The anchoring structure can be any structure that anchors itself and any peptide linked to it to a cell membrane.

In some embodiments of the present disclosure, the cell membrane anchor structure is a polypeptide cell membrane anchor structure comprising an amino acid sequence. The anchoring structure can attach a cytokine to a membrane of a cell. In various embodiments, a cytokine described herein is operatively linked to the anchoring structure. The anchoring structure can comprise a peptide anchor or a non-peptide anchor. The non-peptide anchor can be attached to the polypeptide via a non-peptide anchor attachment signal. The non-peptide anchor attachment signal can be part of the polypeptide. In some cases, the non-peptide anchor attachment signal is operatively linked to a cytokine described herein. In some cases, the non-peptide anchor attachment signal is operatively linked to a cytokine described herein. In some cases, the peptide anchor is operatively linked to a cytokine described herein.

A peptide anchor provided herein can be encoded by a peptide anchor nucleic acid sequence. The peptide anchor nucleic acid sequence can be present as part of the nucleic acid sequences described in the present disclosure, including the nucleic acid sequence in the single cytokine-anchor materials, the first nucleic acid sequence and the second nucleic acid sequence in the two cytokine-anchor materials, and the first nucleic acid sequence, the second nucleic acid sequence, and the third nucleic acid sequence in the three cytokine-anchor materials.

The non-peptide anchor attachment signal can be encoded by a non-peptide anchor attachment signal nucleic acid sequence. The non-peptide anchor attachment signal nucleic acid sequence can be present as part of the nucleic acid sequences described in the present disclosure, including the nucleic acid sequence in the single cytokine-anchor materials, the first nucleic acid sequence and the second nucleic acid sequence in the two cytokine-anchor materials, and the first nucleic acid sequence, the second nucleic acid sequence, and the third nucleic acid sequence in the three cytokine-anchor materials.

### Non-Peptide Anchor

In some embodiments, a non-peptide anchor does not comprise an amino acid sequence. In some embodiments, a non-peptide anchor can be attached to a protein during protein processing. In some embodiments, a non-peptide anchor can be attached to a protein by replacing a non-peptide anchor attachment signal located at a C-terminal of the protein.

A non-peptide anchor provided herein can comprise a lipid, such as a glycolipid. Glycolipids can be lipids with a carbohydrate attached by a glycosidic bond. Glycolipids can maintain stability of the cell membrane. Glycolipids can facilitate cellular recognition. Glycolipids can be located on the surface of all eukaryotic cell membranes. Glycolipids can extend from the phospholipid bilayer into extracellular environment. Glycolipid can comprise glycoglycerolipids and glycosphingolipids. Glycoglycerolipids can comprise acetylated or non-acetylated glycerol with at least one fatty acid as the lipid complex. Glyceroglycolipids can further comprises galactolipids and sulfolipids.

In some cases, the glycolipid comprises a glycosylphosphatidylinositol (GPI). In some cases, the non-peptide anchor comprises a GPI anchor. In some cases, the non-peptide anchor is attached to a non-peptide anchor attachment signal. In some cases, the non-peptide anchor attachment signal comprises a glycolipid-attachment signal. In some cases, the non-peptide anchor attachment signal comprises a GPI-attachment signal. In some cases, the C-terminal GPI attachment signal spans 20-30 amino acids starting from the amino acid to which the GPI attaches after the GPI attachment signal is cleaved off. In some cases, such amino acid to which the GPI attaches comprises Ser, Asn, Asp, Ala, Gly, Cys or Thr. The GPI attachment signal peptide can comprise a stretch of about 10 hydrophilic amino acids. The GPI attachment signal peptide can comprise a stretch of about 20 hydrophobic amino acids.

In some embodiments, a non-peptide anchor can be attached to a C-terminus of the polypeptide during post-translational modification and anchor the cytokine to the cell membrane. In some embodiments, a non-peptide anchor is a glycosylated phosphatidylinositol anchor (GPI anchor). The GPI anchor can comprise a phosphoethanolamine linker, a core glycan, and a phospholipid tail. The structure of the core glycan can be EtNP-6Manα2-Manα6-(EtNP)2Manα4-GINα6-myoIno-P-lipid (EtNP, ethanolamine phosphate: Man, mannose: GlcN, glucosamine; Ino, inositol). In some cases, the GPI anchor can be linked to a C-terminus of the polypeptide via an amide bond generated between the C-terminal carboxyl group and an amino group of the terminal EtNP. In some cases, the core glycan can be modified with side chains, e.g., selected from phosphoethanolamine groups, mannose, galactose, sialic acid or other sugars. Examples of GPI anchor, synthesis, structures and functions thereof are described in Kinoshita Taroh, 2020 Biosynthesis and biology of mammalian GPI-anchored proteins Open Biol. 10190290190290; Paulick M G et al., Biochemistry. 2008; 47 (27); 6991-7000; all of which are herein incorporated by reference in their entirety for all purposes.

Attachment of GPI anchors can be a post-translational modification of proteins that adds glycosylated phosphatidylinositol, which can enable the protein to anchor to the extracellular surface of the cell membrane. In some cases, wild type proteins with GPI anchors do not contain transmembrane or cytoplasmic domains. GPI anchors can be GPI anchors of different families of proteins, including membrane-associated enzymes, adhesion molecules and proteins that coat the outer surface of proto parasites, such as Trypanosoma brucei.

GPI anchors can be attached to the polypeptide by the GPI-attachment signal located at the C-terminus of the polypeptide. After translocation through the endoplasmic reticulum, the GPI-attachment signal can be cleaved off and replaced by a GPI anchor by specific transamidases. Modification of a protein by adding a GPI anchor confers certain properties on the protein, since the added lipid moiety can enable the protein to be inserted into the cell membrane, thereby anchoring the protein. In some embodiments, the GPI anchor is derived from rat brain Thy-1, human erythrocyte AChE, hamster brain scrapie prion protein, human urine CD59, mouse skeletal muscle NCAM, bovine liver 5′-nucleotidase, human placental APase, human CD52, pig kidney membrane dipeptidase, human kidney membrane dipeptidase, Trypanosoma brucei VSG, T. cruzi IG7, T. cruzimucins, T. cruzi NETNES, Leishmania major gp63, Saccharomyces cerevisiae gp125, Aspergillus fumigatus PhoAp, Pyrus communis arabinogalactan proteins, Dictyostelium discoideum PsA, Trypanosoma congolense VSG, or Torpedo AChE.

In some embodiments, a GPI-attachment signal comprises an amino acid sequence that is at least 75%, 80%, 85%, 90%, 95%, 98%, or 99%, or 100% identical to the sequence set forth in SEQ ID NO: 200. In some embodiments, the GPI-attachment signal is encoded by a nucleic acid sequence that is at least 75%, 80%, 85%, 90%, 95%, 98%, or 99%, or 100% identical to any of the sequences set forth in SEQ ID NOs: 250-252. In some embodiments, the GPI-attachment signal is encoded by a nucleic acid sequence that is at least 75%, 80%, 85%, 90%, 95%, 98%, or 99%, or 100% identical to the sequence set forth in SEQ ID NO: 250. In some embodiments, the GPI-attachment signal is encoded by a nucleic acid sequence that is at least 75%, 80%, 85%, 90%, 95%, 98%, or 99%, or 100% identical to the sequence set forth in SEQ ID NO: 251. In some embodiments, the GPI-attachment signal is encoded by a nucleic acid sequence that is at least 75%, 80%, 85%, 90%, 95%, 98%, or 99%, or 100% identical to the sequence set forth in SEQ ID NO: 252.

TABLE 3A
Exemplary non-peptide anchor attachment
signal amino acid sequences

| SEQ | | |
|---|---|---|
| ID | Amino Acid | |
| NO: | Sequence | Description |
| 200 | LENGGTSLSEKTVLL | GPI-attachment signal |
| | LVTPFLAAAWSLHP | (CD59) |
| 200 | LENGGTSLSEKTVLL | GPI-attachment signal |
| | LVTPFLAAAWSLHP | (GPI-1) |
| 200 | LENGGTSLSEKTVLL | GPI-attachment signal |
| | LVTPFLAAAWSLHP | (GPI-2) |

TABLE 3B
Exemplary non-peptide anchor attachment
signal nucleic acid sequences

| SEQ | | |
|---|---|---|
| ID NO: | Nucleic Acid Sequence | Description |
| 250 | cttgaaaatggtgggacatcctta | GPI-attachment |
| | tcagagaaaacagttcttctgctg | signal (CD59) |
| | gtgactccatttctggcagcagcc | |
| | tggagccttcatccc | |
| 251 | ctggagaacggagggacaagcctg | GPI-attachment |
| | agcgagaagacagtgctgctgctg | signal (GPI-1) |
| | gtgaccccatttctggccgcagca | |
| | tggagcctgcacccc | |
| 252 | ctggagaacggggggacaagcctg | GPI-attachment |
| | tccgagaagaccgtgctgctgctg | signal (GPI-2) |
| | gtcactcccttcctggccgccgcc | |
| | tggagcctgcatccc | |

### Peptide Anchor

In some embodiments, the peptide anchor comprises an amino acid sequence that derives from at least a portion of a transmembrane domain of any suitable transmembrane protein. In some embodiments, the peptide anchor comprises a transmembrane peptide sequence.

In some embodiments, the peptide anchor comprises a portion of a transmembrane peptide sequence. In some embodiments, the transmembrane peptide sequence comprises a B7-1 transmembrane amino acid sequence, a B7-2 transmembrane amino acid sequence. B7-H1 transmembrane amino acid sequence. B7-H3 transmembrane amino acid sequence, tumor necrosis factor receptor 2 (TNFR2) transmembrane amino acid sequence, a CD8α transmembrane amino acid sequence, a CD28 transmembrane amino acid sequence, a CD3ζ transmembrane amino acid sequence, a CTLA-4 (CD152) transmembrane amino acid sequence, or a PD-L1 transmembrane amino acid sequence, or any variants thereof. In some embodiments, the peptide anchor comprises an amino acid sequence that is at least 75%, 80%, 85%, 90%, 95%, 98%, or 99%, or 100% identical to the sequence set forth in SEQ ID NO: 203. In some embodiments, the peptide anchor is encoded by a nucleic acid sequence that is at least 75%, 80%, 85%, 90%, 95%, 98%, or 99%, or 100% identical to the sequence set forth in SEQ ID NO: 253. In some embodiments, the peptide anchor comprises an amino acid sequence that is at least 75%, 80%, 85%, 90%, 95%, 98%, or 99%, or 100% identical to the sequence set forth in SEQ ID NO: 204. In some embodiments, the peptide anchor is encoded by a nucleic acid sequence that is at least 75%, 80%, 85%, 90%, 95%, 98%, or 99%, or 100% identical to the sequence set forth in SEQ ID NO: 254. In some embodiments, the peptide anchor is encoded by a nucleic acid sequence that is at least 75%, 80%, 85%, 90%, 95%, 98%, or 99%, or 100% identical to the sequence set forth in SEQ ID NO: 255.

In some embodiments, the peptide anchor comprises at least a portion of transmembrane domains of B7-1, B7-2, CD8, and CD28.

In some embodiments, the peptide anchor comprises at least a portion of B7-1 transmembrane-intracellular amino acid sequence. In some embodiments, the peptide anchor comprises at least a portion of B7-2 transmembrane-intracellular amino acid sequence. In some embodiments, the peptide anchor comprises at least a portion of CD8α transmembrane-intracellular amino acid sequence. In some embodiments, the peptide anchor comprises at least a portion of B7-1 transmembrane amino acid sequence. In some embodiments, the peptide anchor comprises at least a portion of B7-2 transmembrane amino acid sequence. In some embodiments, the peptide anchor comprises at least a portion of CD8α transmembrane amino acid sequence. In some embodiments, the peptide anchor comprises a B7-H1 transmembrane amino acid sequence or any fragments or variants thereof. In some embodiments, the peptide anchor comprises a B7-H3 transmembrane amino acid sequence or any fragments or variants thereof. In some embodiments, the peptide anchor comprises a tumor necrosis factor receptor 2 (TNFR2) transmembrane amino acid sequence or any fragments or variants thereof. In some embodiments, the peptide anchor comprises a CD28 transmembrane amino acid sequence or any fragments or variants thereof. In some embodiments, the peptide anchor comprises a CD3ζ transmembrane amino acid sequence or any fragments or variants thereof. In some embodiments, the peptide anchor comprises a CTLA-4 (CD152) transmembrane amino acid sequence or any fragments or variants thereof. In some embodiments, the peptide anchor comprises a PD-L1 transmembrane amino acid sequence or any fragments or variants thereof.

In some embodiments, the peptide anchor is located at the C-terminal of the polypeptide. In some embodiments, the signal peptide described herein, the cytokine peptide described herein, and the peptide anchor are operably linked in a direction from a N-terminal of the polypeptide to a C-terminal of the polypeptide.

TABLE 4A
Exemplary peptide anchor amino acid sequences

| SEQ | | |
|---|---|---|
| ID | | |
| NO: | Amino Acid Sequence | Description |
| 203 | IYIWAPLAGTCGVLLLSLVIT | transmembrane peptide |
| | | (Ar1) |
| 204 | LLPSWAITLISVNGIFVICCL | transmembrane peptide |
| | TYCFAPRCRERRRNERLRRES | (Ar2) |
| | VRPV | |

TABLE 4B
Exemplary peptide anchor nucleic acid sequences

| SEQ ID | | |
|---|---|---|
| NO: | Nucleic Acid Sequence | Description |
| 253 | atctacatctgggctcctctg | transmembrane |
| | gctggcacctgcggagtgctg | peptide (Ar1) |
| | ctgctgtctctggtgattact | |
| 254 | ctgctgcctagttgggccatc | transmembrane |
| | accctgatcagcgtgaacgga | peptide (Ar2) |
| | atcttcgtgatctgttgtctg | |
| | acatactgttttgctcccagg | |
| | tgcagagagaggagaaggaac | |
| | gagcggctgagaagagaatcc | |
| | gtgcggcctgtg | |
| 255 | ctgctgccttcttgggctatc | transmembrane |
| | accctgatcagcgtgaacgga | peptide (Ar2) |
| | atctttgtgatctgttgtctg | |
| | acttattgtttcgcccccaga | |
| | tgcagagagaggagaaggaat | |
| | gagagactgagaagagaatca | |
| | gtgaggcccgtg | |

### Targeting Moiety

In some aspects, provided herein is a targeting moiety that is present as part of the polypeptides described in the present disclosure, including the polypeptide in the single cytokine-anchor materials, the first polypeptide and the second polypeptide in the two cytokine-anchor materials, and the first polypeptide, the second polypeptide, and the third polypeptide in the three cytokine-anchor materials. The targeting moiety can comprise a CAR, a T cell receptor (TCR), or a B cell receptor (BCR) or fragments thereof.

In some embodiments, the targeting moiety comprises a TCR or fragments thereof. The TCR can be a recombinant TCR, which is generated through recombinant expression of one or more exogenous TCR α-, β-, γ-, and/or δ-chain encoding genes. The recombinant TCR can be a chimeric or hybrid TCR comprised of amino acid sequences of TCRs from two or more mammalian species. The TCR can be a humanized TCR. The TCR can comprise an α-chain. The TCR can comprise a β-chain. The TCR can comprise a γ-chain of a TCR. The TCR can comprise a δ-chain. The polypeptide chains of TCRs are known in the art.

The targeting moiety can be connected to a polypeptide described herein via a cleavable linker described herein. The targeting moiety is encoded by a targeting sequence, which can be connected to a nucleic acid sequence via a cleavable linker nucleic acid sequence. In some cases, the targeting moiety recognizes an antigen. In some cases, the targeting moiety binds to an antigen.

In some cases, the targeting moiety recognizes a tumor-specific antigen. The tumor-specific antigen can be a molecule, including a protein, polypeptide, peptide, lipid, carbohydrate, etc. predominantly expressed or over-expressed by a tumor cell, such that the antigen can be regarded as specifically associated with the tumor or cancer. The tumor-specific antigen can be expressed by normal, non-tumor, or non-cancerous cells but a level that is lower or not as robust as the expression by tumor cells. The tumor cells can over-express the tumor-specific antigen or express the tumor-specific antigen at a significantly higher level than that by normal, non-cancerous cells. The tumor-specific antigen can be expressed by cells of a different state of development or maturation. For example, the tumor-specific antigen can be expressed by cells of the embryonic or fetal stage, which cells are not normally found in an adult subject. The tumor-specific antigen can be expressed by stem cells or precursor cells, which are not normally found in an adult subject. In some cases, tumor-specific antigen can be a mutated antigen that is predominantly expressed or overexpressed by tumor or cancer cells and not expressed or expressed at a significantly lower level by normal, non-cancerous cells.

Examples of tumor-specific antigen include mesothelin, gp100, CD19, CD20, CD22, CD30, CD33, CD38, CD70, CD123, CD138, CD276, CD171, CD5, CD7, MUC1, AFP, CEA, PSCA, PSMA, HER2, EGFR, IL13Ralpha2, GD2, NKG2D, EGFTvIII, CS1, CCL1, BCMA, Mesothelin, ROR1, FLT3, FAP, TAG72, CD38, CD44v6, CEA, EPCAM, B7H3, KIT, IL-13Ralpha2, PRSS21, VEGR2, LewisY, CD24, PDGFR-beta, SSEA-4, AFP, NCAM, Claudin18.2, GPC3, GM3, TGS5, HMWMAA, TEM7R, CLDN6, GPRC5D, CXORF61, CD97, CD179a, ALK, PLAC1, GloboH, NY-BR-1, UPK2, HAVCR1, ADRB3, PANX3, GPR20, LY6K, OR51E2, TRAP, WT1, NY-ESO-1, LAGE-1a, MAGE-A1, MAGE-A2. BRCA, MAGE-A3, MAGE-A4, MAGE-A5, MAGE-A6, MAGE-A7, MAGE-A8, MAGE-A9, MAGE-A10, MAGE-A11, MAGE-A12, EGFRVIII, VEGFR-2, TRP-1, TRP-2, tyrosinase, human papillomavirus (HPV) 16 E6, HPV 16 E7, HPV 18 E6, HPV 18 E7, KK-LC-1, NY-BR-, NY-ESO-1 (or CAG-3), SSX-2, SSX-3, SSX-4, SSX-5, SSX-9, SSX-1. In some embodiments, the tumor-specific antigen is CD19.

In some embodiments, the targeting moiety comprises a chimeric antigen receptor (CAR). A CAR can comprise a ligand binding domain, a transmembrane domain, a co-stimulatory domain, and an intracellular signaling domain.

In some embodiments, the ligand binding domain recognizes the tumor-specific antigen described herein. In some embodiments, the ligand binding domain binds to the tumor-specific antigen described herein. In some embodiments, the ligand binding domain is derived from an antibody or antibody fragment (e.g., a murine, human or humanized antibody) that recognizes the tumor-specific antigen described herein.

In some embodiments, the CAR targets CD19, CD20, CD22, CD30, CD33, CD38, CD123, CD138, CD171, CD5, CD7, MUC1, AFP, CEA, PSCA, PSMA, Her2, EGFR, IL13Rα2, GD2, NKG2D, EGFRvIII, CS1, CCL1, BCMA, Mesothelin, ROR1, FLT3, FAP, TAG72, CD38, CD44v6, CEA, EPCAM, B7H3, KIT, IL-13Ra2, PRSS21, VEGFR2, LewisY, CD24, PDGFR-β, SSEA-4, AFP, NCAM, Claudin18.2, GM3, TGS5, HMWMAA, TEM7R, CLDN6, GPRC5D, CXORF61, CD97, CD179a, ALK, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1, or any combination thereof.

In some embodiments, the ligand binding domain of CAR binds CD19, CD20, CD22, CD30, CD33, CD38, CD123, CD138, CD171, CD5, CD7, MUC1, AFP, CEA, PSCA, PSMA, Her2, EGFR, IL13Rα2, GD2, NKG2D, EGFRvIII, CS1, CCL1, BCMA, Mesothelin, ROR1, FLT3, FAP, TAG72, CD38, CD44v6, CEA, EPCAM, B7H3, KIT, IL-13Ra2, PRSS21, VEGFR2, LewisY, CD24, PDGFR-β, SSEA-4, AFP, NCAM, Claudin18.2, GM3, TGS5, HMWMAA, TEM7R, CLDN6, GPRC5D, CXORF61, CD97, CD179a, ALK, PLAC1, GloboH, NY-BR-1, UPK2, HAVCR1, ADRB3, PANX3, GPR20, LY6K, OR51E2, TARP, WT1, NY-ESO-1, LAGE-1a, MAGE-A1 or any combination thereof.

In some embodiments, the ligand binding domain is a scFv fragment. In some embodiments, the ligand binding domain targets CD19. In some embodiments, the ligand binding domain comprises an amino acid sequence that is at least 75%, 80%, 85%, 90%, 95%, 98%, or 99%, or 100% identical to the sequence set forth in SEQ ID NO: 401. In some embodiments, the ligand binding domain comprises an amino acid sequence that is at least 75% identical to the sequence set forth in SEQ ID NO: 401. In some embodiments, the ligand binding domain comprises an amino acid sequence that is at least 80% identical to the sequence set forth in SEQ ID NO: 401. In some embodiments, the ligand binding domain comprises an amino acid sequence that is at least 85% identical to the sequence set forth in SEQ ID NO: 401. In some embodiments, the ligand binding domain comprises an amino acid sequence that is at least 90% identical to the sequence set forth in SEQ ID NO: 401. In some embodiments, the ligand binding domain comprises an amino acid sequence that is at least 95% identical to the sequence set forth in SEQ ID NO: 401. In some embodiments, the ligand binding domain comprises an amino acid sequence that is at least 98% identical to the sequence set forth in SEQ ID NO: 401. In some embodiments, the ligand binding domain comprises an amino acid sequence that is at least 99% identical to the sequence set forth in SEQ ID NO: 401. In some embodiments, the ligand binding domain comprises an amino acid sequence that is 100% identical to the sequence set forth in SEQ ID NO: 401. In some embodiments, the ligand binding domain is encoded by a nucleic acid sequence that is at least 75%, 80%, 85%, 90%, 95%, 98%, or 99%, or 100% identical to the sequence set forth in SEQ ID NO: 451. In some embodiments, the ligand binding domain is encoded by a nucleic acid sequence that is at least 75% identical to the sequence set forth in SEQ ID NO: 451. In some embodiments, the ligand binding domain is encoded by a nucleic acid sequence that is at least 80% identical to the sequence set forth in SEQ ID NO: 451. In some embodiments, the ligand binding domain is encoded by a nucleic acid sequence that is at least 85% identical to the sequence set forth in SEQ ID NO: 451. In some embodiments, the ligand binding domain is encoded by a nucleic acid sequence that is at least 90% identical to the sequence set forth in SEQ ID NO: 451. In some embodiments, the ligand binding domain is encoded by a nucleic acid sequence that is at least 95% identical to the sequence set forth in SEQ ID NO: 451. In some embodiments, the ligand binding domain is encoded by a nucleic acid sequence that is at least 98% identical to the sequence set forth in SEQ ID NO: 451. In some embodiments, the ligand binding domain is encoded by a nucleic acid sequence that is at least 99% identical to the sequence set forth in SEQ ID NO: 451. In some embodiments, the ligand binding domain is encoded by a nucleic acid sequence that is 100% identical to the sequence set forth in SEQ ID NO: 451.

In some cases, CAR comprises a leader. The leader can locate at the N-terminal of the CAR. The leader can be connected to the ligand binding domain. In some embodiments, the leader comprises the leader of CD8α or a variant thereof. In some embodiments, the leader comprises the signal peptide of β2M or a variant thereof. In some embodiments, the leader comprises an amino acid sequence that is at least 75%, 80%, 85%, 90%, 95%, 98%, or 99%, or 100% identical to the sequence set forth in SEQ ID NO: 400. In some embodiments, the leader comprises an amino acid sequence that is at least 75%, 80%, 85%, 90%, 95%, 98%, or 99%, or 100% identical to the sequence set forth in SEQ ID NO: 4. In some embodiments, the leader is encoded by a nucleic acid sequence that is at least 75%, 80%, 85%, 90%, 95%, 98%, or 99%, or 100% identical to the sequence set forth in SEQ ID NO: 450. In some embodiments, the leader is encoded by a nucleic acid sequence that is at least 75%, 80%, 85%, 90%, 95%, 98%, or 99%, or 100% identical to the sequence set forth in SEQ ID NO: 54.

In some cases, CAR comprises a hinge region. The hinge region can connect the ligand binding domain and the transmembrane domain. In some cases, the hinge region is from a human protein. In some embodiments, the hinge region comprises hinge region of human Ig hinge, such as IgG1 IgG4, IgD, FcγRIIIα, a KIR2DS2 hinge, or CD8α hinge. In some embodiments, the hinge region comprises a peptide linker described herein, for example, a GS linker. In some embodiments, the hinge region comprises an amino acid sequence that is at least 75%, 80%, 85%, 90%, 95%, 98%, or 99%, or 100% identical to the sequence set forth in SEQ ID NO: 402. In some embodiments, the hinge region is encoded by a nucleic acid sequence that is at least 75%, 80%, 85%, 90%, 95%, 98%, or 99%, or 100% identical to the sequence set forth in SEQ ID NO: 452.

In some cases, CAR comprises a transmembrane domain. The transmembrane domain can be the transmembrane domain of TCRα chain, TCRβ chain, TCRγ chain, TCRδ chain, CD3ζ subunit, CD3ε subunit, CD3γ subunit, CD3δ subunit CD45, CD4, CD5, CD8α, CD9, CD16, CD22, CD33, CD28, CD37, CD64, CD80, CD86, CD123, CD134, CD137, CD154, or any combination thereof. In some embodiments, the transmembrane domain comprises a transmembrane domain of TCRα chain, TCRβ chain, TCRγ chain, TCRδ chain, CD3ζ subunit, CD3ε subunit, CD3γ subunit, CD3δ subunit CD45, CD4, CD5, CD8α, CD9, CD16, CD22, CD33, CD28, CD37, CD64, CD80, CD86, CD123, CD134, CD137, CD154, or any fragments or variants thereof.

In some embodiments, the transmembrane domain comprises the transmembrane domain of CD8α or fragments thereof. In some embodiments, the transmembrane domain comprises an amino acid sequence that is at least 75%, 80%, 85%, 90%, 95%, 98%, or 99%, or 100% identical to the sequence set forth in SEQ ID NO: 403. In some embodiments, the transmembrane domain is encoded by a nucleic acid sequence that is at least 75%, 80%, 85%, 90%, 95%, 98%, or 99%, or 100% identical to the sequence set forth in SEQ ID NO: 453.

In some cases, CAR comprises a co-stimulatory domain. In some cases, the co-stimulatory domain comprises at least a portion of TLR1, TLR2, TLR3, TLR4, TLR5, TLR6, TLR7, TLR8, TLR9, TLR10, CARD11, CD2, CD7, CD8, CD16, CD18, CD27, CD28, CD30, CD40, CD54, CD83, CD134(OX40), CD137(4-1BB), CD270(HVEM), CD272(BTLA), CD276(B7-H3), CD278(ICOS), CD357(GITR), DAP10, DAP12, LAT, NKG2C, SLP76, PD-1, LIGHT, TRIM, ZAP70, LFA-1, CD38, or any combination thereof. In other cases, the co-stimulatory domain comprises at least a portion of any other costimulatory domain with an immunoreceptor tyrosine-based activation motif. In some embodiments, the co-stimulatory domain is a co-stimulatory domain of CD28. In some embodiments, the co-stimulatory domain is a co-stimulatory domain of CD137 (4-1BB). In some embodiments, the co-stimulatory domain comprises an amino acid sequence that is at least 75%, 80%, 85%, 90%, 95%, 98%, or 99%, or 100% identical to the sequence set forth in SEQ ID NO: 404. In some embodiments, the co-stimulatory domain is encoded by a nucleic acid sequence that is at least 75%, 80%, 85%, 90%, 95%, 98%, or 99%, or 100% identical to the sequence set forth in SEQ ID NO: 454.

In some cases, CAR comprises an intracellular signaling domain. The intracellular signaling domain can comprise at least a portion of an intracellular signaling domain from FcRγ, FcRβ, CD3γ, CD3δ, CD3ε, CD3ζ, CD22, CD79a, CD79b, or CD66d. In some embodiments, the intracellular signaling domain comprises the intracellular signaling domain of CD34. In some embodiments, the intracellular signaling domain comprises an amino acid sequence that is at least 75%, 80%, 85%, 90%, 95%, 98%, or 99%, or 100% identical to the sequence set forth in SEQ ID NO: 405. In some embodiments, the intracellular signaling domain is encoded by a nucleic acid sequence that is at least 75%, 80%, 85%, 90%, 95%, 98%, or 99%, or 100% identical to the sequence set forth in SEQ ID NO: 455.

In some embodiments, the CAR comprises an amino acid sequence that is at least 75%, 80%, 85%, 90%, 95%, 98%, or 99%, or 100% identical to any of the sequences set forth in SEQ ID NOs: 4 or 400-407. In some embodiments, the CAR comprises an amino acid sequence that is at least 75% identical to any of the sequences set forth in SEQ ID NOs: 4 or 400-407. In some embodiments, the CAR comprises an amino acid sequence that is at least 80% identical to any of the sequences set forth in SEQ ID NOs: 4 or 400-407. In some embodiments, the CAR comprises an amino acid sequence that is at least 85% identical to any of the sequences set forth in SEQ ID NOs: 4 or 400-407. In some embodiments, the CAR comprises an amino acid sequence that is at least 90% identical to any of the sequences set forth in SEQ ID NOs: 4 or 400-407. In some embodiments, the CAR comprises an amino acid sequence that is at least 95% identical to any of the sequences set forth in SEQ ID NOs: 4 or 400-407. In some embodiments, the CAR comprises an amino acid sequence that is at least 98% identical to any of the sequences set forth in SEQ ID NOs: 4 or 400-407. In some embodiments, the CAR comprises an amino acid sequence that is at least 99% identical to any of the sequences set forth in SEQ ID NOs: 4 or 400-407. In some embodiments, the CAR comprises an amino acid sequence that is 100% identical to any of the sequences set forth in SEQ ID NOs: 4 or 400-407. In some embodiments, the CAR comprises an amino acid sequence that is at least 75%, 80%, 85%, 90%, 95%, 98%, or 99%, or 100% identical to the sequence set forth in SEQ ID NO: 406. In some embodiments, the CAR comprises an amino acid sequence that is at least 80% identical to the sequence set forth in SEQ ID NO: 406. In some embodiments, the CAR comprises an amino acid sequence that is at least 85% identical to the sequence set forth in SEQ ID NO: 406. In some embodiments, the CAR comprises an amino acid sequence that is at least 90% identical to the sequence set forth in SEQ ID NO: 406. In some embodiments, the CAR comprises an amino acid sequence that is at least 95% identical to the sequence set forth in SEQ ID NO: 406. In some embodiments, the CAR comprises an amino acid sequence that is 100% identical to the sequence set forth in SEQ ID NO: 406. In some embodiments, the CAR comprises an amino acid sequence that is at least 75%, 80%, 85%, 90%, 95%, 98%, or 99%, or 100% identical to the sequence set forth in SEQ ID NO: 407.

In some embodiments, the CAR is encoded by a nucleic acid sequence that is at least 75%, 80%, 85%, 90%, 95%, 98%, or 99%, or 100% identical to any of the sequences set forth in SEQ ID NOs: 54 or 450-457. In some embodiments, the CAR is encoded by a nucleic acid sequence that is at least 75% identical to any of the sequences set forth in SEQ ID NOs: 54 or 450-457. In some embodiments, the CAR is encoded by a nucleic acid sequence that is at least 80% identical to any of the sequences set forth in SEQ ID NOs: 54 or 450-457. In some embodiments, the CAR is encoded by a nucleic acid sequence that is at least 85% identical to any of the sequences set forth in SEQ ID NOs: 54 or 450-457. In some embodiments, the CAR is encoded by a nucleic acid sequence that is at least 90% identical to any of the sequences set forth in SEQ ID NOs: 54 or 450-457. In some embodiments, the CAR is encoded by a nucleic acid sequence that is at least 95% identical to any of the sequences set forth in SEQ ID NOs: 54 or 450-457. In some embodiments, the CAR is encoded by a nucleic acid sequence that is at least 98% identical to any of the sequences set forth in SEQ ID NOs: 54 or 450-457. In some embodiments, the CAR is encoded by a nucleic acid sequence that is at least 99% identical to any of the sequences set forth in SEQ ID NOs: 54 or 450-457. In some embodiments, the CAR is encoded by a nucleic acid sequence that is 100% identical to any of the sequences set forth in SEQ ID NOs: 54 or 450-457. In some embodiments, the CAR is encoded by a nucleic acid sequence that is at least 80% identical to the sequences set forth in SEQ ID NO: 456. In some embodiments, the CAR is encoded by a nucleic acid sequence that is at least 80% identical to the sequences set forth in SEQ ID NO: 456. In some embodiments, the CAR is encoded by a nucleic acid sequence that is at least 85% identical to the sequences set forth in SEQ ID NO: 456. In some embodiments, the CAR is encoded by a nucleic acid sequence that is at least 90% identical to the sequences set forth in SEQ ID NO: 456. In some embodiments, the CAR is encoded by a nucleic acid sequence that is at least 95% identical to the sequences set forth in SEQ ID NO: 456. In some embodiments, the CAR is encoded by a nucleic acid sequence that is 100% identical to the sequences set forth in SEQ ID NO: 456. In some embodiments, the CAR is encoded by a nucleic acid sequence that is 100% identical to the sequences set forth in SEQ ID NO: 457.

TABLE 5A
Exemplary CAR sequence amino acid sequences

| SEQ ID | | |
|---|---|---|
| NO: | Amino Acid Sequence | Description |
| 400 | ALPVTALLLPLALLLHAARP | Leader (CD8α) |
| 4 | SRSVALAVLALLSLSGLEA | Leader (β2M) |
| 401 | DIQMTQTTSSLSASLGDRVTISCRASQDISKYLNWYQQKPDGTVKLLI | scFv (CD19) |
| | YHTSRLHSGVPSRFSGSGSGTDYSLTISNLEQEDIATYFCQQGNTLPY | |
| | TFGGGTKLEITGGGGSGGGGSGGGGSEVKLQESGPGLVAPSQSLSVTC | |
| | TVSGVSLPDYGVSWIRQPPRKGLEWLGVIWGSETTYYNSALKSRLTII | |
| | KDNSKSQVFLKMNSLQTDDTAIYYCAKHYYYGGSYAMDYWGQGTSVTV | |
| | SS | |
| 402 | TTTPAPRPPTPAPTIASQPLSLRPEACRPAAGGAVHTRGLDFACD | Hinge (CD8α) |
| 403 | IYIWAPLAGTCGVLLLSLVITLYC | Transmembrane |
| | | domain (CD8α) |
| 404 | KRGRKKLLYIFKQPFMRPVQTTQEEDGCSCRFPEEEEGGCEL | Costimulatory |
| | | domain (4-1BB) |
| 405 | RVKFSRSADAPAYQQGQNQLYNELNLGRREEYDVLDKRRGRDPEMGGK | Intracellular signal |
| | PRRKNPQEGLYNELQKDKMAEAYSEIGMKGERRRGKGHDGLYQGLSTA | domain (CD3ζ) |
| | TKDTYDALHMQALPPR | |
| 406 | ALPVTALLLPLALLLHAARPDIQMTQTTSSLSASLGDRVTISCRASQD | Complete sequence |
| | ISKYLNWYQQKPDGTVKLLIYHTSRLHSGVPSRFSGSGSGTDYSLTIS | of CAR (CD8α |
| | NLEQEDIATYFCQQGNTLPYTFGGGTKLEITGGGGSGGGGSGGGGSEV | Leader + Anti |
| | KLQESGPGLVAPSQSLSVTCTVSGVSLPDYGVSWIRQPPRKGLEWLGV | CD19-scFv + CD8α |
| | IWGSETTYYNSALKSRLTIIKDNSKSQVFLKMNSLQTDDTAIYYCAKH | Hinge + CD8α |
| | YYYGGSYAMDYWGQGTSVTVSSTTTPAPRPPTPAPTIASQPLSLRPEA | Transmembrane + 4- |
| | CRPAAGGAVHTRGLDFACDIYIWAPLAGTCGVLLLSLVITLYCKRGRK | 1BB + CD3ζ) |
| | KLLYIFKQPFMRPVQTTQEEDGCSCRFPEEEEGGCELRVKFSRSADAP | |
| | AYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQ | |
| | ALPPR | |
| 407 | SRSVALAVLALLSLSGLEADIQMTQTTSSLSASLGDRVTISCRASQDISKYLN | Complete sequence |
| | WYQQKPDGTVKLLIYHTSRLHSGVPSRESGSGSGTDYSLTISNLEQEDIATYF | of CAR (B2M |
| | CQQGNTLPYTFGGGTKLEITGGGGSGGGGSGGGGSEVKLQESGPGLVAPSQSL | Leader + Anti |
| | SVTCTVSGVSLPDYGVSWIRQPPRKGLEWLGVIWGSETTYYNSALKSRLTIIK | CD19-scFv + CD8α |
| | DNSKSQVFLKMNSLQTDDTAIYYCAKHYYYGGSYAMDYWGQGTSVTVSSTTTP | Hinge + CD8α |
| | APRPPTPAPTIASQPLSLRPEACRPAAGGAVHTRGLDFACDIYIWAPLAGTCG | Transmembrane + 4- |
| | VLLLSLVITLYCKRGRKKLLYIFKQPFMRPVQTTQEEDGCSCRFPEEEEGGCE | 1BB + CD3ζ) |
| | LRVKFSRSADAPAYQQGQNQLYNELNLGRREEYDVLDKRRGRDPEMGGKPRRK | |
| | NPQEGLYNELQKDKMAEAYSEIGMKGERRRGKGHDGLYQGLSTATKDTYDALH | |
| | MQALPPR | |

TABLE 5B
Exemplary CAR sequence nucleic acid sequences

| SEQ ID | | |
|---|---|---|
| NO: | Nucleic Acid Sequence | Description |
| 450 | gccttaccagtgaccgccttgctcctgccgctggccttgctgctccacgc | Leader (CD8α) |
| | cgccaggccg | |
| 54 | tctcgctccgtggccttagctgtgctcgcgctactctctctttctggcct | Leader (B2M) |
| | ggaggct | |
| 451 | gacatccagatgacacagactacatcctccctgtctgcctctctgggaga | scFv (CD19) |
| | cagagtcaccatcagttgcagggcaagtcaggacattagtaaatatttaa | |
| | attggtatcagcagaaaccagatggaactgttaaactcctgatctaccat | |
| | acatcaagattacactcaggagtcccatcaaggttcagtggcagtgggtc | |
| | tggaacagattattctctcaccattagcaacctggagcaagaagatattg | |
| | ccacttacttttgccaacagggtaatacgcttccgtacacgttcggaggg | |
| | gggaccaagctggagatcacaggtggcggtggctcgggcggtggtgggtc | |
| | gggtggcggcggatctgaggtgaaactgcaggagtcaggacctggcctgg | |
| | tggcgccctcacagagcctgtccgtcacatgcactgtctcaggggtctca | |
| | ttacccgactatggtgtaagctggattcgccagcctccacgaaagggtct | |
| | ggagtggctgggagtaatatggggtagtgaaaccacatactataattcag | |
| | ctctcaaatccagactgaccatcatcaaggacaactccaagagccaagtt | |
| | ttcttaaaaatgaacagtctgcaaactgatgacacagccatttactactg | |
| | tgccaaacattattactacggtggtagctatgctatggactactggggcc | |
| | aaggaacctcagtcaccgtctcctca | |
| 452 | accacgacgccagcgccgcgaccaccaacaccggcgcccaccatcgcgtc | Hinge (CD8α) |
| | gcagcccctgtccctgcgcccagaggcgtgccggccagcggcggggggcg | |
| | cagtgcacacgagggggctggacttcgcctgtgat | |
| 453 | atctacatctgggcgcccttggccgggacttgtggggtccttctcctgtc | Transmembrane |
| | actggttatcaccctttactgc | domain (CD8α) |
| 454 | aaacggggcagaaagaaactcctgtatatattcaaacaaccatttatgag | Costimulatory |
| | accagtacaaactactcaagaggaagatggctgtagctgccgatttccag | domain (4-1BB) |
| | aagaagaagaaggaggatgtgaactg | |
| 455 | agagtgaagttcagcaggagcgcagacgcccccgcgtaccagcagggcca | Intracellular |
| | gaaccagctctataacgagctcaatctaggacgaagagaggagtacgatg | signal domain |
| | ttttggacaagagacgtggccgggaccctgagatggggggaaagccgaga | (CD39) |
| | aggaagaaccctcaggaaggcctgtacaatgaactgcagaaagataagat | |
| | ggcggaggcctacagtgagattgggatgaaaggcgagcgccggaggggca | |
| | aggggcacgatggcctttaccagggtctcagtacagccaccaaggacacc | |
| | tacgacgcccttcacatgcaggccctgccccctcgc | |
| 456 | gccttaccagtgaccgccttgctcctgccgctggccttgctgctccacgc | Complete |
| | cgccaggccggacatccagatgacacagactacatcctccctgtctgcct | sequence of CAR |
| | ctctgggagacagagtcaccatcagttgcagggcaagtcaggacattagt | (CD8α |
| | aaatatttaaattggtatcagcagaaaccagatggaactgttaaactcct | Leader + Anti |
| | gatctaccatacatcaagattacactcaggagtcccatcaaggttcagtg | CD19- |
| | gcagtgggtctggaacagattattctctcaccattagcaacctggagcaa | scFv + CD8α |
| | gaagatattgccacttacttttgccaacagggtaatacgcttccgtacac | Hinge + CD8α |
| | gttcggaggggggaccaagctggagatcacaggtggcggtggctcgggcg | Transmembrane+ |
| | gtggtgggtcgggggcggcggatctgaggtgaaactgcaggagtcagga | 4-1BB + CD3ζ) |
| | cctggcctggtggcgccctcacagagcctgtccgtcacatgcactgtctc | |
| | aggggtctcattacccgactatggtgtaagctggattcgccagcctccac | |
| | gaaagggtctggagtggctgggagtaatatggggtagtgaaaccacatac | |
| | tataattcagctctcaaatccagactgaccatcatcaaggacaactccaa | |
| | gagccaagttttcttaaaaatgaacagtctgcaaactgatgacacagcca | |
| | tttactactgtgccaaacattattactacggtggtagctatgctatggac | |
| | tactggggccaaggaacctcagtcaccgtctcctcaaccacgacgccagc | |
| | gccgcgaccaccaacaccggcgcccaccatcgcgtcgcagcccctgtccc | |
| | tgcgcccagaggcgtgccggccagcggcggggggcgcagtgcacacgagg | |
| | gggctggacttcgcctgtgatatctacatctgggcgcccttggccgggac | |
| | ttgtggggtccttctcctgtcactggttatcaccctttactgcaaacggg | |
| | gcagaaagaaactcctgtatatattcaaacaaccatttatgagaccagta | |
| | caaactactcaagaggaagatggctgtagctgccgatttccagaagaaga | |
| | agaaggaggatgtgaactgagagtgaagttcagcaggagcgcagacgccc | |
| | ccgcgtaccagcagggccagaaccagctctataacgagctcaatctagga | |
| | cgaagagaggagtacgatgttttggacaagagacgtggccgggaccctga | |
| | gatggggggaaagccgagaaggaagaaccctcaggaaggcctgtacaatg | |
| | aactgcagaaagataagatggcggaggcctacagtgagattgggatgaaa | |
| | ggcgagcgccggaggggcaaggggcacgatggcctttaccagggtctcag | |
| | tacagccaccaaggacacctacgacgcccttcacatgcaggccctgcccc | |
| | ctcgc | |
| 457 | tctcgctccgtggccttagctgtgctcgcgctactctctctttctggcct | Complete |
| | ggaggctgacatccagatgacacagactacatcctccctgtctgcctctc | sequence of CAR |
| | tgggagacagagtcaccatcagttgcagggcaagtcaggacattagtaaa | (B2M Leader + |
| | tatttaaattggtatcagcagaaaccagatggaactgttaaactcctgat | Anti CD19-scFv |
| | ctaccatacatcaagattacactcaggagtcccatcaaggttcagtggca | + CD8α Hinge + |
| | gtgggtctggaacagattattctctcaccattagcaacctggagcaagaa | CD8α |
| | gatattgccacttacttttgccaacagggtaatacgcttccgtacacgtt | Transmembrane |
| | cggaggggggaccaagctggagatcacaggtggcggtggctcgggcggtg | + 4-1BB + |
| | gtgggtcgggtggcggcggatctgaggtgaaactgcaggagtcaggacct | CD3ζ) |
| | ggcctggtggcgccctcacagagcctgtccgtcacatgcactgtctcagg | |
| | ggtctcattacccgactatggtgtaagctggattcgccagcctccacgaa | |
| | agggtctggagtggctgggagtaatatggggtagtgaaaccacatactat | |
| | aattcagctctcaaatccagactgaccatcatcaaggacaactccaagag | |
| | ccaagttttcttaaaaatgaacagtctgcaaactgatgacacagccattt | |
| | actactgtgccaaacattattactacggtggtagctatgctatggactac | |
| | tggggccaaggaacctcagtcaccgtctcctcaaccacgacgccagcgcc | |
| | gcgaccaccaacaccggcgcccaccatcgcgtcgcagcccctgtccctgc | |
| | gcccagaggcgtgccggccagcggcggggggcgcagtgcacacgaggggg | |
| | ctggacttcgcctgtgatatctacatctgggcgcccttggccgggacttg | |
| | tggggtccttctcctgtcactggttatcaccctttactgcaaacggggca | |
| | gaaagaaactcctgtatatattcaaacaaccatttatgagaccagtacaa | |
| | actactcaagaggaagatggctgtagctgccgatttccagaagaagaaga | |
| | aggaggatgtgaactgagagtgaagttcagcaggagcgcagacgcccccg | |
| | cgtaccagcagggccagaaccagctctataacgagctcaatctaggacga | |
| | agagaggagtacgatgttttggacaagagacgtggccgggaccctgagat | |
| | ggggggaaagccgagaaggaagaaccctcaggaaggcctgtacaatgaac | |
| | tgcagaaagataagatggcggaggcctacagtgagattgggatgaaaggc | |
| | gagcgccggaggggcaaggggcacgatggcctttaccagggtctcagtac | |
| | agccaccaaggacacctacgacgcccttcacatgcaggccctgccccctc | |
| | gc | |

### Cleavable Linker Sequence

In some aspects, provided herein are cleavable linkers that can connect one or more components described in the present disclosure, including the targeting moiety and the polypeptide in the single cytokine-anchor materials, the targeting moiety, the first polypeptide, and the second polypeptide in the two cytokine-anchor materials, and the targeting moiety, the first polypeptide, the second polypeptide, and the third polypeptide in the three cytokine-anchor materials.

In some embodiments, the cleavable linker connects the targeting moiety and the signal peptide of the polypeptide. In some embodiments, the cleavable linker connects the targeting moiety and the peptide anchor of the polypeptide. In some embodiments, the cleavable linker connects the targeting moiety and the non-peptide anchor attachment signal. In some embodiments, the cleavable linker connects the first polypeptide and the second polypeptide. In some embodiments, the cleavable linker connects the first polypeptide and the third polypeptide. In some embodiments, the cleavable linker connects the third polypeptide and the second polypeptide.

The cleavable linker can be encoded by a cleavable linker nucleic acid sequence. The cleavable linker nucleic acid sequence can connect two or more nucleic acid sequences described in the present disclosure, including the targeting sequence and the nucleic acid sequence in the single cytokine-anchor materials, the targeting sequence, the first nucleic acid sequence, and the second nucleic acid sequence in the two cytokine-anchor materials, and the targeting sequence, the first nucleic acid sequence, the second nucleic acid sequence, and the third nucleic acid sequence in the three cytokine-anchor materials.

In some embodiments, the second nucleic acid sequence and the first nucleic acid sequence are linked via a cleavable linker nucleic acid sequence. In some embodiments, the third nucleic acid sequence and the first nucleic acid sequence are linked via a cleavable linker nucleic acid sequence. In some embodiments, the second nucleic acid sequence and the third nucleic acid sequence are linked via a cleavable linker nucleic acid sequence. In some embodiments, the targeting sequence and the first nucleic acid sequence are linked via a cleavable linker nucleic acid sequence. In some embodiments, the targeting sequence and the second nucleic acid sequence are linked via a cleavable linker nucleic acid sequence. In some embodiments, the targeting sequence and the third nucleic acid sequence are linked via a cleavable linker nucleic acid sequence.

In some embodiments, the cleavable linker sequence between the first nucleic acid sequence encoding the first polypeptide and the second nucleic acid sequence encoding the second polypeptide can be cleaved off, thus, producing two polypeptides: a first polypeptide comprising a first cytokine and a first anchoring structure; and a second polypeptide comprising a second cytokine and a second anchoring structure. In some embodiments, the cleavable linker sequence between the second nucleic acid sequence encoding the second polypeptide and the third nucleic acid sequence encoding the third polypeptide can be cleaved off, thus, producing two polypeptides: a second polypeptide comprising a second cytokine and a second anchoring structure; and a third polypeptide comprising a third cytokine and a second anchoring structure. In some embodiments, the cleavable linker sequence between the first nucleic acid sequence encoding the first polypeptide and the third nucleic acid sequence encoding the third polypeptide can be cleaved off, thus, producing two polypeptides: a first polypeptide comprising a first cytokine and a first anchoring structure; and a third polypeptide comprising a third cytokine and a second anchoring structure.

In some embodiments, the cleavable linker sequence between the first nucleic acid sequence encoding the first polypeptide and the targeting sequence encoding a targeting moiety can be cleaved off, thus, producing two polypeptides: a first polypeptide comprising a first cytokine and a first anchoring structure; and a targeting moiety comprising a CAR, such as a CAR19 (e.g., having a sequence set forth in SEQ ID NO: 406). In some embodiments, the cleavable linker sequence between the second nucleic acid sequence encoding the second polypeptide and the targeting sequence encoding a targeting moiety can be cleaved off, thus, producing two polypeptides: a second polypeptide comprising a second cytokine and a second anchoring structure; and a targeting moiety comprising a CAR, such as a CAR19. In some embodiments, the cleavable linker sequence between the third nucleic acid sequence encoding the third polypeptide and the targeting sequence encoding a targeting moiety can be cleaved off, thus, producing two polypeptides: a third polypeptide comprising a third cytokine and a third anchoring structure; and a targeting moiety comprising a CAR, such as a CAR19.

The cleavable linker can be any cleavable linker that connects two peptides.

In some embodiments, the cleavable linker is not limited in length. The cleavable linker can comprise about 20 to about 30 amino acid residues, for example, about 20, about 21, about 22, about 23, about 24, about 25, about 26, about 27, about 28, about 29, about 30 amino acid residues.

In some cases, the cleavable linker comprises an IRES element, such as an IRES element is from encephalomyocarditis virus (EMCV). In some cases, the cleavable linker comprises a self-cleaving 2A peptide. A 2A peptide can be a viral oligopeptide that mediates polypeptide cleavage during translation in eukaryotic cells. The term “2A” refers to a specific region of the viral genome.

Without wishing to be bound by a certain theory, the mechanism of 2A-mediated self-cleavage can be ribosomal “skipping” of glycyl-prolyl peptide bond formation at the C-terminus of the 2A peptide rather than true proteolytic cleavage.

In some embodiments, the cleavable linker comprises a 2A self-cleaving peptides or 2A-like peptides from foot-and-mouth virus or cardiovirus. In some embodiments, the cleavable linker comprises a 2A peptide sequence, including amino acid sequence of porcine Czech virus-1 2A (P2A), the amino acid sequence of equine rhinitis A virus (E2A), the amino acid sequence of β tetrasomy virus 2A (T2A), amino acid sequence or foot-and-mouth disease virus (F2A) amino acid sequence. In some embodiments, the cleavable linker comprises a P2A peptide sequence. In some embodiments, the cleavable linker comprises a T2A peptide sequence. In some embodiments, the cleavable linker comprises a E2A peptide sequence. In some embodiments, the cleavable linker comprises a F2A peptide sequence. In some embodiments, the cleavable linker comprises an IRES peptide. In some embodiments, the cleavable linker comprises an amino acid sequence that is at least 75%, 80%, 85%, 90%, 95%, 98%, or 99%, or 100% identical to any of the sequences set forth in SEQ ID NOs: 300-303. In some embodiments, the cleavable linker comprises an amino acid sequence that is at least 75%, 80%, 85%, 90%, 95%, 98%, or 99%, or 100% identical to the sequence set forth in SEQ ID NO: 300. In some embodiments, the cleavable linker comprises an amino acid sequence that is at least 75%, 80%, 85%, 90%, 95%, 98%, or 99%, or 100% identical to the sequence set forth in SEQ ID NO: 301. In some embodiments, the cleavable linker comprises an amino acid sequence that is at least 75%, 80%, 85%, 90%, 95%, 98%, or 99%, or 100% identical to the sequence set forth in SEQ ID NO: 302. In some embodiments, the cleavable linker comprises an amino acid sequence that is at least 75%, 80%, 85%, 90%, 95%, 98%, or 99%, or 100% identical to the sequence set forth in SEQ ID NO: 303.

In some embodiments, the cleavable linker is encoded by the cleavable linker nucleic acid sequence comprises a nucleic acid sequence that is at least 75%, 80%, 85%, 90%, 95%, 98%, or 99%, or 100% identical to any of the sequences set forth in SEQ ID NOs: 350-355. In some embodiments, the cleavable linker is encoded by the cleavable linker nucleic acid sequence comprises a nucleic acid sequence that is at least 75%, 80%, 85%, 90%, 95%, 98%, or 99%, or 100% identical to the sequence set forth in SEQ ID NO: 350. In some embodiments, the cleavable linker is encoded by the cleavable linker nucleic acid sequence comprises a nucleic acid sequence that is at least 75%, 80%, 85%, 90%, 95%, 98%, or 99%, or 100% identical to the sequence set forth in SEQ ID NO: 351. In some embodiments, the cleavable linker is encoded by the cleavable linker nucleic acid sequence comprises a nucleic acid sequence that is at least 75%, 80%, 85%, 90%, 95%, 98%, or 99%, or 100% identical to the sequence set forth in SEQ ID NO: 352. In some embodiments, the cleavable linker is encoded by the cleavable linker nucleic acid sequence comprises a nucleic acid sequence that is at least 75%, 80%, 85%, 90%, 95%, 98%, or 99%, or 100% identical to the sequence set forth in SEQ ID NO: 353. In some embodiments, the cleavable linker is encoded by the cleavable linker nucleic acid sequence comprises a nucleic acid sequence that is at least 75%, 80%, 85%, 90%, 95%, 98%, or 99%, or 100% identical to the sequence set forth in SEQ ID NO: 354. In some embodiments, the cleavable linker is encoded by the cleavable linker nucleic acid sequence comprises a nucleic acid sequence that is at least 75%, 80%, 85%, 90%, 95%, 98%, or 99%, or 100% identical to the sequence set forth in SEQ ID NO: 355.

In other cases, the cleavable linker comprises a furin cleavage site or a tobacco etch virus cleavage site. Furin cleavage sites and sequences are described in Klimstra et al., J Virol. 1999 August; 73 (8); 6299-6306; U.S. Pat. No. 8,871,906; all of which are herein incorporated by reference in their entirety for all purposes.

TABLE 6A
Exemplary cleavable linker amino acid sequences

| SEQ ID NO: | Amino Acid Sequence | Description |
|---|---|---|
| 300 | ATNFSLLKQAGDVEENPGP | P2A peptide |
| 301 | EGRGSLLTCGDVEENPGP | T2A peptide |
| 302 | QCTNYALLKLAGDVESNPGP | E2A peptide |
| 303 | RAKRSGSGATNFSLLKQAGDVEENPGP | F2A peptide |

TABLE 6B
Exemplary cleavable linker nucleic acid sequences

| SEQ ID | | |
|---|---|---|
| NO: | Nucleic Acid Sequence | Description |
| 350 | gccacgaacttctctctgttaaagcaagcaggagacgtggaagaaaaccc | P2A peptide |
| | cggtccc | |
| 351 | gagggcaggggaagtctactaacatgcggggacgtggaggaaaatcccgg | T2A peptide |
| | cccc | |
| 352 | cagtgtaccaattatgctctgctgaagctggctggggacgtggaaagcaa | E2A peptide |
| | ccccggacct | |
| 353 | agggccaagaggagcggaagcggggctacaaacttcagtctgctgaagca | F2A peptide |
| | ggctggcgacgtggaggaaaaccctggacct | |
| 354 | cccctctccctcccccccccctaacgttactggccgaagccgcttggaat | IRES |
| | aaggccggtgtgcgtttgtctatatgttattttccaccatattgccgtct | peptide |
| | tttggcaatgtgagggcccggaaacctggccctgtcttcttgacgagcat | |
| | tcctaggggtctttcccctctcgccaaaggaatgcaaggtctgttgaatg | |
| | tcgtgaaggaagcagttcctctggaagcttcttgaagacaaacaacgtct | |
| | gtagcgaccctttgcaggcagcggaaccccccacctggcgacaggtgcct | |
| | ctgcggccaaaagccacgtgtataagatacacctgcaaaggcggcacaac | |
| | cccagtgccacgttgtgagttggatagttgtggaaagagtcaaatggctc | |
| | tcctcaagcgtattcaacaaggggctgaaggatgcccagaaggtacccca | |
| | ttgtatgggatctgatctggggcctcggtgcacatgctttacatgtgttt | |
| | agtcgaggttaaaaaaacgtctaggccccccgaaccacggggacgtggtt | |
| | ttcctttgaaaaacacgatgataa | |
| 355 | gccacgaacttctctctgttaaagcaagcaggagatgttgaagaaaaccc | P2A peptide |
| | cgggcct | |

### Peptide Linker

In some aspects, provided herein are peptide linkers that can connect one or more components described in the present disclosure, including the cytokine and the anchoring structure of the polypeptide in the single cytokine-anchor materials, the cytokine and the anchoring structure of the first polypeptide and the second polypeptide in the two cytokine-anchor materials, and the cytokine and the anchoring structure of the first polypeptide, the second polypeptide, and the third polypeptide in the three cytokine-anchor materials.

In some embodiments, the peptide linker connects the cytokine and the anchoring structure. In some embodiments, the peptide linker connects the cytokine and the peptide anchor. In some embodiments, the peptide linker connects the cytokine and the non-peptide anchor attachment signal. In some embodiments, the peptide linker connects the peptide anchor to the cleavable linker. In some embodiments, the peptide linker connects the non-peptide anchor attachment signal to the cleavable linker.

A peptide linker provided herein has no particular limitation. In some cases, a peptide linker in the polypeptide provided herein is a flexible peptide linker. In some cases, a peptide linker in the polypeptide provided herein is an inflexible peptide linker. In some cases, a peptide linker in the polypeptide provided herein is not a self-cleavable linker. A peptide linker can be any suitable linker sequence that connects the anchoring structure to any other components. A peptide linker can be any suitable linker sequence that connects the anchoring structure to the cytokine. A peptide linker can be any suitable linker sequence that connects the anchoring structure to the cleavable linker, which further connects to another polypeptide or a targeting moiety.

In some embodiments, the peptide linker has no particular limitation in its length. In some embodiments, the peptide linker comprises about 2 to about 10 amino acid residues. In some embodiments, the peptide linker comprises about 10 to about 65 amino acid residues, about 18 to about 61 amino acid residues, or about 25 to about 50 amino acid residues. In some embodiments, the peptide linker comprises about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, or about 65 amino acid residues.

In some embodiments, the peptide linker comprises a GS linker, a Lr1 linker, or a Lr8 linker.

In some embodiments, the peptide linker comprises glycine and serine residues. In some embodiments, a peptide linker comprises one or more repeats of GAS (SEQ ID NO: 710) or G₃S (SEQ ID NO: 711), for example, about 3 to about 15 or about 5 to about 12 repeats of GAS (SEQ ID NO: 710) and G₃S (SEQ ID NO: 711). In some embodiments, a peptide linker comprises about 3, about 4, about 5, about 6, about 7, about 8, about 9, about 10, about 11, about 12, about 13, about 14, or about 15 repeats of GAS (SEQ ID NO: 710). In some embodiments, a peptide linker comprises about 3, about 4, about 5, about 6, about 7, about 8, about 9, about 10, about 11, about 12, about 13, about 14, or about 15 repeats of G₃S (SEQ ID NO: 711).

In some embodiments, the peptide linker comprises glycine polymer (G)ₙ. In some embodiments, the peptide linker comprises glycine-serine polymer (G₁₋₅S₁₋₅)n, wherein n is 1, 2, 3, 4 or 5 (SEQ ID NO: 700). In some embodiments, the peptide linker comprises glycine-alanine polymer. In some embodiments, the peptide linker comprises alanine-serine polymer. In some embodiments, the peptide linker comprises GGG. In some embodiments, the peptide linker comprises DGGGS (SEQ ID NO: 701). In some embodiments, the peptide linker comprises TGEKP (SEQ ID NO: 702). In some embodiments, the peptide linker comprises (GGGGS)ₙ, where n=1, 2, 3, 4, or 5 (SEQ ID NO: 703). In some embodiments, the peptide linker comprises EGKSSGSGSESKVD (SEQ ID NO: 704). In some embodiments, the peptide linker comprises KESGSVSSEQLAQFRSLD (SEQ ID NO: 705). In some embodiments, the peptide linker comprises GGRRGGGS (SEQ ID NO: 706). In some embodiments, the peptide linker comprises LRQRDGERP (SEQ ID NO: 707). In some embodiments, the peptide linker comprises LRQKDGGGSERP (SEQ ID NO: 708). In some embodiments, the peptide linker comprises LRQKD(GGGS)₂ERP (SEQ ID NO: 709).

In some embodiments, the peptide linker comprises an amino acid sequence that is at least 75%, 80%, 85%, 90%, 95%, 98%, or 99%, or 100% identical to any of the sequences set forth in SEQ ID NOs: 500, 501, 504, 506, or 507, or the sequence of LE, AS, GSG, or EF. In some embodiments, the peptide linker comprises an amino acid sequence that is at least 75%, 80%, 85%, 90%, 95%, 98%, or 99%, or 100% identical to the sequence set forth in SEQ ID NO: 500. In some embodiments, the peptide linker comprises an amino acid sequence that is at least 75%, 80%, 85%, 90%, 95%, 98%, or 99%, or 100% identical to the sequence set forth in SEQ ID NO: 501. In some embodiments, the peptide linker comprises the sequence of LE. In some embodiments, the peptide linker comprises the sequence of AS. In some embodiments, the peptide linker comprises an amino acid sequence set forth in SEQ ID NO: 504. In some embodiments, the peptide linker comprises the sequence of GSG. In some embodiments, the peptide linker comprises an amino acid sequence that is at least 75%, 80%, 85%, 90%, 95%, 98%, or 99%, or 100% identical to the sequence set forth in SEQ ID NO: 506. In some embodiments, the peptide linker comprises an amino acid sequence that is at least 75%, 80%, 85%, 90%, 95%, 98%, or 99%, or 100% identical to the sequence set forth in SEQ ID NO: 507. In some embodiments, the peptide linker comprises the sequence of EF. In some embodiments, the peptide linker is encoded by a nucleic acid sequence that is at least 75%, 80%, 85%, 90%, 95%, 98%, or 99%, or 100% identical to the sequence of gagttc. In some embodiments, the peptide linker is encoded by a nucleic acid sequence that is at least 75%, 80%, 85%, 90%, 95%, 98%, or 99%, or 100% identical to the sequence set forth in SEQ ID NO: 509. In some embodiments, the peptide linker is encoded by a nucleic acid sequence that is at least 75%, 80%, 85%, 90%, 95%, 98%, or 99%, or 100% identical to the sequence set forth in SEQ ID NO: 520. In some embodiments, the peptide linker is encoded by a nucleic acid sequence that is at least 75%, 80%, 85%, 90%, 95%, 98%, or 99%, or 100% identical to the sequence of ggctccggc. In some embodiments, the peptide linker is encoded by a nucleic acid sequence that is at least 75%, 80%, 85%, 90%, 95%, 98%, or 99%, or 100% identical to the sequence of ggaagcgga.

Other examples of peptide linkers are described in Liu et al., PNAS, pp. 5525-5530 (1997); Pomerantz et al., Proc. Natl. Acad. Sci. USA Vol. 92, pp. 9752-9756, October 1995; Kim et al., PNAS, Vol. 93, pp. 1156-1160 (1996); Chaudhary et al. 1990, Proc. Natl. Acad. Sci. USA, 87: 1066-1070; Bird et al., 1988. Science, No. 242: pp. 423-426; all of which are herein incorporated by reference in their entirety for all purposes.

TABLE 7A
Exemplary peptide linker amino acid sequences

| SEQ ID NO: | Amino Acid Sequence | Description |
|---|---|---|
| 500 | RAEF | |
| 501 | AGTRGSG | |
| | LE | |
| | AS | |
| 504 | AGIDGSG | |
| | GSG | |
| 506 | SGGGGSGGGGSGGGGSGGGGSGGGSLQ | Lr1 |
| 507 | SGGGGSGGGGSGGGGSGGGGSGGGGSGGGGSGGGGSGGGG | Lr8 |
| | SGGGGSLQ | |
| | EF | |

TABLE 7B
Exemplary peptide linker nucleic acid sequences

| SEQ ID NO: | Nucleic Acid Sequence | Description |
|---|---|---|
| | ggctccggc | GSG |
| | ggaagcgga | GSG |
| | gagttc | EF |
| 509 | tctggaggaggaggatctggcggaggaggaagtggaggaggagg | Lr1 |
| | ctctggaggaggcggatctggaggagggagtctgcag | |
| 520 | agtggcgggggaggatctggaggcggaggatctgggggaggagg | Lr1 |
| | aagcggaggaggagggagcggaggaggcagcctgcag | |

### Nucleic Acid Molecules, Vectors, and Systems

According to aspects of the present disclosure, provided herein are nucleic acid molecules encoding the polypeptides described in the present disclosure.

From the primary amino acid sequence of the polypeptide(s) or any components thereof encoding the cytokine-anchor protein construct provided herein, the person of skill in the art is able to determine suitable nucleotide sequence(s) that encodes the polypeptide(s) and, if desired, one that is codon-optimized (e.g., see Mauro and Chappell. Trends Mol Med. 20 (11); 604-613, 2014).

The nucleic acid molecule(s) that encode the polypeptides in the cytokine-anchor materials, such as according to some embodiments of the disclosure, may be, or may be part of, a vector (such as a plasmid vector, cosmid vector or viral vector, or an artificial chromosome) that may comprise other functional regions (elements) such as one or more promoters, one or more origins or replication, one or more selectable marker(s), and one or more other elements typically found in expression vectors. The cloning and expression of nucleic acids that encode proteins, including CAR and cytokines, is well established and well within the skill of the person in the art.

In some embodiments, the nucleic acid molecules of the cytokine-anchor materials are greater than 80%, such as greater than 90%, greater than 95%, greater than 97% and greater than 99% pure.

In some aspects, provided herein is a vector comprising one or more of the nucleic acid sequences described in the present disclosure. Vector can be a transfer vector, which refers to composition of matter which comprises an isolated nucleic acid and which can be used to deliver the isolated nucleic acid to the interior of a cell. Numerous vectors are known in the art including linear polynucleotides, polynucleotides associated with ionic or amphiphilic compounds, plasmids, and viruses. “Transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include adenoviral vectors, adeno-associated virus vectors, retroviral vectors, lentiviral vectors, and the like. Vector can also include an expression vector, which refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

In some embodiments, the nucleic acid molecule described herein is a vector. In some embodiments, the vector is a viral vector. In some embodiments, the vector is a retroviral vector, a lentiviral vector, an adenoviral vector, or an adeno-associated viral vector.

In some embodiments, the vector is a retroviral vector. A retroviral vector generally refers to an RNA virus that can reverse transcribe a DNA complementary strand in an infected cell, and use this DNA single strand as a template to synthesize a second DNA strand and incorporate it into the cell genome in DNA. The retroviral vector can use host cell enzymes to transcribe and replicate RNA to synthesize proteins, repackage the virus, and release it from the cell to become an infectious virus. The transduction efficiency of the retrovirus can be high, and the transfection rate of the gene can be effectively improved via the retroviral vector.

In some embodiments, the vector is a lentiviral vector. A lentiviral vector refers to the gene therapy vector developed on the basis of HIV-1 (human immunodeficiency type I virus). The lentiviral vector can infect both dividing cells and non-dividing cells. It can effectively infect almost all mammalian cells including neuron cells, liver cells, etc., with high infection efficiency. Lentiviruses can efficiently integrate foreign genes into the host chromosomes to achieve persistent expression.

In some embodiments, the vector is a transposon plasmid. A transposon plasmid generally refers to the basic unit existing on chromosomal DNA and capable of autonomous replication and displacement. The transposon plasmid can “jump” from one position of the genome to another through a series of processes such as cutting and reintegration.

In some embodiments, the vector is an expression vector. In some embodiments, the expression vector comprises a nucleic acid sequence encoding a targeting moiety (e.g., a CAR). In some embodiments, the expression vector comprises a nucleic acid sequence encoding a cytokine and an anchoring structure. In some embodiments, the expression vector comprises a nucleic acid sequence encoding a CAR, a cytokine, and an anchoring structure. In some embodiments, the expression vector comprises a second nucleic acid sequence encoding a second cytokine and a second anchoring structure. In some embodiments, the expression vector comprises a second nucleic acid sequence encoding a second CAR, a second cytokine and a second anchoring structure. In some embodiments, the expression vector comprises a third nucleic acid sequence encoding a third cytokine and a third anchoring structure. In some embodiments, the expression vector comprises a third nucleic acid sequence encoding a third CAR, a third cytokine and a third anchoring structure. In some embodiments, the nucleic acid sequences within the expression vector can be arranged upstream or downstream. Specifically, relative to the nucleic acid encoding the CAR molecule, the nucleic acid encoding the cytokine and the anchoring structure may be arranged upstream or downstream. The nucleic acid encoding the CAR and the nucleic acid encoding the cytokine may be linked via a cleavable linker nucleic acid sequence encoding a 2A peptide or an IRES.

In some embodiments, the nucleic acid molecules and/or vector of the present disclosure is introduced into a host cell. For eukaryotic cells, for example, suitable techniques include calcium phosphate transfection. DEAE-Dextran, electroporation, liposome-mediated transfection and transduction using retrovirus or other virus, e.g., vaccinia or, for insect cells, baculovirus. In some cases, introducing nucleic acid in the host cell, in particular a eukaryotic cell, uses a viral or a plasmid-based system. In some cases, the plasmid system is maintained episomally. In other cases, the plasmid system is incorporated into the host cell or into an artificial chromosome. In a particular embodiment, the incorporation is by random integration of one or more copies at single or multiple loci. In some embodiments, the incorporation is by targeted integration of one or more copies at single or multiple loci. For bacterial cells, suitable techniques include, for example, calcium chloride transformation, electroporation and transfection using bacteriophage.

In some embodiments, nucleic acid sequences are disposed on the same vectors. In some embodiments, the two or more nucleic acid sequences are encoded by a single nucleic molecule in the same frame and as a single polypeptide chain. In some cases, the targeting moiety (e.g., CAR) and the cytokine-anchoring structure can be separated by one or more peptide cleavage sites. (e.g., an auto-cleavage site or a substrate for an intracellular protease). In some cases, the targeting sequence is under control of a different promoter as the nucleic acid sequence or the exogenous nucleic acid sequence. In some cases, the targeting sequence is under control of a same promoter as the nucleic acid sequence or the exogenous nucleic acid sequence. In some cases, the targeting sequence and the first nucleic acid sequence are under control of a same promoter. In some cases, targeting sequence and the first nucleic acid sequence are under control of two different promoters. In some cases, the targeting sequence and the second nucleic acid sequence are under control of a same promoter. In some cases, targeting sequence and the second nucleic acid sequence are under control of two different promoters. In some cases, the targeting sequence and the third nucleic acid sequence are under control of a same promoter. In some cases, targeting sequence and the third nucleic acid sequence are under control of two different promoters. In some cases, the second nucleic acid sequence and the first nucleic acid sequence are under control of a same promoter. In some cases, the second nucleic acid sequence and the first nucleic acid sequence are under control of two different promoters. In some cases, the second nucleic acid sequence and the third nucleic acid sequence are under control of a same promoter. In some cases, the second nucleic acid sequence and the third nucleic acid sequence are under control of two different promoters. In some cases, the third nucleic acid sequence and the first nucleic acid sequence are under control of a same promoter. In some cases, the third nucleic acid sequence and the first nucleic acid sequence are under control of two different promoters.

In some cases, the first nucleic acid sequence, the second nucleic acid sequence, and the third nucleic acid sequence, are operably linked in a 5′ to 3′ direction. In some cases, the first nucleic acid sequence, the third nucleic acid sequence, and the second nucleic acid sequence are operably linked in a 5′ to 3′ direction. In some cases, the second nucleic acid sequence, the first nucleic acid sequence, and the third nucleic acid sequence, are operably linked in a 5′ to 3′ direction. In some cases, the second nucleic acid sequence, the third nucleic acid sequence, and the first nucleic acid sequence are operably linked in a 5′ to 3′ direction. In some cases, the third nucleic acid sequence, the second nucleic acid sequence, and the first nucleic acid sequence are operably linked in a 5′ to 3′ direction. In some cases, the third nucleic acid sequence, the first nucleic acid sequence, and the second nucleic acid sequence are operably linked in a 5′ to 3′ direction.

In some cases, the targeting sequence, the first nucleic acid sequence, the second nucleic acid sequence, and the third nucleic acid sequence, are operably linked in a 5′ to 3′ direction. In some cases, the targeting sequence, the first nucleic acid sequence, the third nucleic acid sequence, and the second nucleic acid sequence are operably linked in a 5′ to 3′ direction. In some cases, the targeting sequence, the second nucleic acid sequence, the first nucleic acid sequence, and the third nucleic acid sequence, are operably linked in a 5′ to 3′ direction. In some cases, the targeting sequence, the second nucleic acid sequence, the third nucleic acid sequence, and the first nucleic acid sequence are operably linked in a 5′ to 3′ direction. In some cases, the targeting sequence, the third nucleic acid sequence, the second nucleic acid sequence, and the first nucleic acid sequence are operably linked in a 5′ to 3′ direction. In some cases, the targeting sequence, the third nucleic acid sequence, the first nucleic acid sequence, and the second nucleic acid sequence are operably linked in a 5′ to 3′ direction.

In some cases, the first nucleic acid sequence, the second nucleic acid sequence, the third nucleic acid sequence, and the targeting sequence, are operably linked in a 5′ to 3′ direction. In some cases, the first nucleic acid sequence, the third nucleic acid sequence, the second nucleic acid sequence, and the targeting sequence, are operably linked in a 5′ to 3′ direction. In some cases, the second nucleic acid sequence, the first nucleic acid sequence, the third nucleic acid sequence and the targeting sequence, are operably linked in a 5′ to 3′ direction. In some cases, the second nucleic acid sequence, the third nucleic acid sequence, the first nucleic acid sequence, and the targeting sequence, are operably linked in a 5′ to 3′ direction. In some cases, the third nucleic acid sequence, the second nucleic acid sequence, the first nucleic acid sequence, and the targeting sequence, are operably linked in a 5′ to 3′ direction. In some cases, the third nucleic acid sequence, the first nucleic acid sequence, the second nucleic acid sequence, and the targeting sequence, are operably linked in a 5′ to 3′ direction.

In other cases, nucleic acid sequences are disposed on separate vectors.

In some embodiments, the nucleic acid of the present disclosure is integrated into the genome (e.g., chromosome) of the host cell. In a particular embodiment, integration is promoted by inclusion of sequences that promote recombination with the genome, in accordance with standard techniques. In some embodiments, the targeting sequence and the nucleic acid sequence are present in a genome of the immune cell. In some embodiments, the targeting sequence, the first nucleic acid sequence, and the second nucleic acid sequence are present in a genome of the cell. In some embodiments, the targeting sequence, the first nucleic acid sequence, the second nucleic acid sequence, and the third nucleic acid sequence are present in a genome of the cell.

According to aspects of the present disclosure, provided herein are systems relating to the cytokine-anchor materials described herein. A system can comprise the nucleic acid molecule or the vector described herein. In some embodiments, a system comprises one nucleic acid molecule or one plasmid, which encodes for targeting moiety, one or more cytokines that are each connected to an anchoring structure. In some embodiments, a system with two nucleic acid molecules is provided, e.g., a dual-plasmid system, where a first plasmid contains a targeting sequence encoding a CAR, and a second plasmid contains a nucleic acid sequence encoding a cytokine and an anchoring structure. For example, the first and the second nucleic acid molecules are co-delivered into a host cell, such as an engineered immune cell. In some embodiments, a system with two or more nucleic acid molecules is provided, where two or more plasmids independently contain at least one targeting sequence encoding a CAR, or at least two or more nucleic acid sequences encoding a cytokine and an anchoring structure. For example, the three or more nucleic acid molecules are co-delivered into a host cell, such as an engineered immune cell. In some cases, a first plasmid contains a targeting sequence, a second plasmids contains a first nucleic acid sequence encoding a first cytokine and a first anchoring structure with or without a targeting sequence, and a third plasmids contains a second nucleic acid sequence encoding a second cytokine and a second anchoring structure with or without a targeting sequence. In some cases, a first plasmid contains a first nucleic acid sequence encoding first cytokine and a first anchoring structure with or without a targeting sequence, a second plasmids contains a second nucleic acid sequence encoding a second cytokine and a second anchoring structure with or without a targeting sequence, and a third plasmids contains a third nucleic acid sequence encoding a third cytokine and a third anchoring structure with or without a targeting sequence. In some cases, a first plasmid contains a first nucleic acid sequence encoding a first cytokine and a first anchoring structure and a second nucleic acid sequence encoding a second cytokine and a second anchoring structure with or without a targeting sequence, a second plasmids contains a targeting sequence, and a third plasmids contains a third nucleic sequence containing a third cytokine and a third anchoring structure with or without a targeting sequence.

In some embodiments, the targeting sequence and the first nucleic acid sequence are present in a same plasmid inside the system. In some embodiments, the targeting sequence and the first nucleic acid sequence are present in two different plasmids inside the system. In some embodiments, the targeting sequence and the second nucleic acid sequence are present in a same plasmid inside the system. In some embodiments, the targeting sequence and the second nucleic acid sequence are present in two different plasmids inside the system. In some embodiments, the first nucleic acid sequence and the second nucleic acid sequence are present in a same plasmid inside the system. In some embodiments, the first nucleic acid sequence and the second nucleic acid sequence are present in two different plasmids inside the system. In some embodiments, the targeting sequence and the first nucleic acid sequence are present in a same plasmid inside the system. In some embodiments, the targeting sequence and the first nucleic acid sequence are present in two different plasmids inside the system. In some embodiments, the targeting sequence and the second nucleic acid sequence are present in a same plasmid inside the system. In some embodiments, the targeting sequence and the second nucleic acid sequence are present in two different plasmids inside the system. In some embodiments, the targeting sequence and the third nucleic acid sequence are present in a same plasmid inside the system. In some embodiments, the targeting sequence and the third nucleic acid sequence are present in two different plasmids inside the system. In some embodiments, the first nucleic acid sequence and the second nucleic acid sequence are present in a same plasmid inside the system. In some embodiments, the first nucleic acid sequence and the second nucleic acid sequence are present in two different plasmids inside the system. In some embodiments, the first nucleic acid sequence and the third nucleic acid sequence are present in a same plasmid inside the system. In some embodiments, the first nucleic acid sequence and the third nucleic acid sequence are present in two different plasmids inside the system. In some embodiments, the second nucleic acid sequence and the third nucleic acid sequence are present in a same plasmid inside the system. In some embodiments, the second nucleic acid sequence and the third nucleic acid sequence are present in two different plasmids inside the system.

A system can comprise proteins or polypeptides encoded by the nucleic acid sequences or nucleic acid molecules inside the system described herein.

### Host Cells

A further aspect of the present disclosure provides a host cell containing nucleic acid molecules, vectors, or systems as disclosed herein. In some embodiments, such a host cell is in vitro. In some embodiments, such a host cell is in culture.

In some cases, the host cell is from any species, such as a bacterium or yeast. In other cases, the host cell is a mammalian cell such as a human cell or rodent cell, for example an engineered T cell or an engineered NK cell. In some cases, the host cell can be any T cell, for example, a cultured T cell, a primary T cell, a T cell from a cultured T cell line (e.g., Jurkat, SupT1, etc.), or a T cell obtained from a mammal. In some cases, the T cell is obtained from a mammal. In some cases, the T cell is obtained from numerous sources, including blood, bone marrow, lymph node, the thymus, or other tissues or fluids. T cells can be enriched for or purified. In some cases, the T cell is a human T cell. In some cases, the T cell is a T cell isolated from a human. The T cell can be any type of T cell and can be of any developmental stage, including CD4+/CD8+ double positive T cells, CD4+ helper T cells, e.g., Th1 and Th2 cells. CD4+ T cells, CD8+ T cells (e.g., cytotoxic T cells), tumor infiltrating lymphocytes (TILs), memory T cells (e.g., central memory T cells and effector memory T cells), naïve T cells, and the like.

In some cases, the host cell is treated so as to cause or allow expression of the protein of the cytokine-anchor materials from the nucleic acid molecules, e.g., by culturing host cells under conditions for expression of the encoding nucleic acid sequences. In some embodiments, the purification of the expressed product is achieved by methods known to one of skill in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acid molecules are well-known in the art. See, for example, Sambrook et al., 2012, MOLECULAR CLONING: A LABORATORY MANUAL, volumes 1-4, Cold Spring Harbor Press, NY). In some cases, a method for the introduction of a polynucleotide into a host cell is calcium phosphate transfection

Biological methods for introducing a polynucleotide of the present disclosure into a host cell include the use of DNA and RNA vectors. Viral vectors, and especially retroviral vectors, have become the most widely used method for inserting genes into mammalian, e.g., human cells. Other viral vectors can come from lentivirus, poxviruses, herpes simplex virus I, adenoviruses and adeno-associated viruses, and the like. Sec, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid molecules associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma. St. Louis, Mo.; dicetyl phosphate (“DCP”) can be obtained from K & K Laboratories (Plainview. N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham. Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce nucleic acid molecules, vectors, or systems described herein into a host cell, in order to confirm the presence of the recombinant DNA sequence in the host cell, a variety of assays may be performed. Such assays include, for example, “molecular biological” assays well known to those of skill in the art, such as Southern and Northern blotting. RT-PCR and PCR; “biochemical” assays, such as detecting the presence or absence of a particular peptide, e.g., by immunological means (ELISAs and Western blots) or by assays described herein to identify agents falling within the scope of the present disclosure.

The present disclosure provides a vector comprising a targeting sequence encoding a CAR and a nucleic acid sequence encoding a cytokine and an anchoring structure. In some cases, the vector can be directly transduced into a cell, e.g., a T cell or NK cell. In some cases, the vector is capable of expressing the CAR construct in mammalian T cells or NK cells. In one aspect, the mammalian T cell is a human T cell.

In some embodiments, the nucleic acid molecules of the present disclosure, including vector nucleic acids that comprise nucleic acid sequences that encode the polypeptides for the CAR or cytokine-anchor of the present disclosure, is present in an isolated host cell. In some cases, the host cell is part of a clonal population of host cells. As used herein, reference to a host cell also encompasses a clonal population of the cell. A clonal population is one that has been grown from a single parent host cell. In some cases, the host cell is from any suitable organism. In some embodiments, the host cell is, for example, bacterial, yeast, fungal or mammalian cells. In some embodiments, the host cell is an immune cell or a tumor cell. In some embodiments, the host cell is an engineered immune cell. In some embodiments, the host cell is a T cell. In some embodiments, the host cell is a tumor infiltrating lymphocyte (TIL). In some embodiments, the engineered immune cell is a natural killer (NK) cell.

In some embodiments, the targeting sequence and the first nucleic acid sequence are present in a same plasmid inside the cell. In some embodiments, the targeting sequence and the first nucleic acid sequence are present in two different plasmids inside the cell. In some embodiments, the targeting sequence and the second nucleic acid sequence are present in a same plasmid inside the cell. In some embodiments, the targeting sequence and the second nucleic acid sequence are present in two different plasmids inside the cell. In some embodiments, the first nucleic acid sequence and the second nucleic acid sequence are present in a same plasmid inside the cell. In some embodiments, the first nucleic acid sequence and the second nucleic acid sequence are present in two different plasmids inside the cell. In some embodiments, the targeting sequence and the first nucleic acid sequence are present in a same plasmid inside the cell. In some embodiments, the targeting sequence and the first nucleic acid sequence are present in two different plasmids inside the cell. In some embodiments, the targeting sequence and the second nucleic acid sequence are present in a same plasmid inside the cell. In some embodiments, the targeting sequence and the second nucleic acid sequence are present in two different plasmids inside the cell. In some embodiments, the targeting sequence and the third nucleic acid sequence are present in a same plasmid inside the cell. In some embodiments, the targeting sequence and the third nucleic acid sequence are present in two different plasmids inside the cell. In some embodiments, the first nucleic acid sequence and the second nucleic acid sequence are present in a same plasmid inside the cell. In some embodiments, the first nucleic acid sequence and the second nucleic acid sequence are present in two different plasmids inside the cell. In some embodiments, the first nucleic acid sequence and the third nucleic acid sequence are present in a same plasmid inside the cell. In some embodiments, the first nucleic acid sequence and the third nucleic acid sequence are present in two different plasmids inside the cell. In some embodiments, the second nucleic acid sequence and the third nucleic acid sequence are present in a same plasmid inside the cell. In some embodiments, the second nucleic acid sequence and the third nucleic acid sequence are present in two different plasmids inside the cell.

### Sources of Cells

Prior to expansion and genetic modification or other modification, a source of cells, e.g., T cells or natural killer (NK) cells, can be obtained directly or indirectly from a subject. Examples of subjects include humans, monkeys, chimpanzees, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some cases of the present disclosure, immune effector cells, e.g., T cells, can be obtained from a unit of blood collected from a subject using any number of techniques known to the skilled artisan, such as Ficoll™ separation. In some cases, cells from the circulating blood of an individual are obtained by apheresis. The apheresis product can contain lymphocytes, including T cells, monocytes, granulocytes. B cells, other nucleated white blood cells, red blood cells, and platelets. In some cases, the cells collected by apheresis can be washed to remove the plasma fraction and, optionally, to place the cells in an appropriate buffer or media for subsequent processing steps. In some cases, the cells are washed with phosphate buffered saline (PBS). In other cases, the wash solution lacks calcium and can lack magnesium or can lack many if not all divalent cations. Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells can be resuspended in a variety of biocompatible buffers, such as, for example, Ca-free, Mg-free PBS, PlasmaLyte A, or other saline solution with or without buffer. Alternatively, the undesirable components of the apheresis sample can be removed and the cells directly resuspended in culture media.

In some cases, T cells are isolated from peripheral blood lymphocytes by lysing the red blood cells and depleting the monocytes, for example, by centrifugation through a PERCOLL™ gradient or by counterflow centrifugal elutriation.

The methods can include, e.g., selection of a specific subpopulation of immune effector cells, e.g., T cells, that are a T regulatory cell-depleted population. CD25+ depleted cells, using, e.g., a negative selection technique. Preferably, the population of T regulatory depleted cells contains less than 30%, 25%, 20%, 15%, 10%, 5%, 4%, 3%, 2%, 1% of CD25+ cells.

In some embodiments. T regulatory cells, e.g., CD25+ T cells, are removed from the population using an anti-C25 antibody, or fragment thereof, or a CD25-binding ligand. IL-2. In some embodiments, the anti-CD25 antibody, or fragment thereof, or CD25-binding ligand is conjugated to a substrate, e.g., a bead, or is otherwise coated on a substrate, e.g., a bead. In some embodiments, the anti-CD25 antibody, or fragment thereof, is conjugated to a substrate as described herein.

In some embodiments, the T regulatory cells, e.g., CD25+ T cells, are removed from the population using CD25 depletion reagent from Militenyi™.

In some embodiments, the T regulatory cells, e.g., CD25+ cells, are removed from the population using the CliniMAC system with a depletion tubing set, such as, e.g., tubing 162-01. In some embodiments, the CliniMAC system is run on a depletion setting such as, e.g., DEPLETION2.1.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

Without wishing to be bound by a particular theory, providing an immune cell with CAR and cytokine-anchor as described herein to a subject can reduce the risk of subject relapse while reducing or eliminating the need to decrease the level of negative regulators of immune cells (e.g., decreasing the number of unwanted immune cells, e.g., TREG cells) in a subject prior to apheresis or during manufacturing of a CAR-expressing cell product, which also reduces the risk of subject relapse. In some cases, providing an immune cell with CAR and cytokine-anchor as described herein to a subject can reduce the risk of subject relapse without the process of eliminating certain immune cells in the subject (i.e., without the lymphodepletion process). Common methods of depleting TREG cells are known in the art. Methods of decreasing TREG cells include cyclophosphamide, anti-GITR antibody (an anti-GITR antibody described herein), CD25-depletion, and combinations thereof.

In some embodiments, the manufacturing methods do not comprise reducing the number of (e.g., depleting) TREG cells prior to manufacturing of the CAR-expressing cell. In some embodiments, the manufacturing methods comprise reducing a certain number of (e.g., partially depleting) TREG cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to partially deplete TREG cells prior to manufacturing of the CAR-expressing cell (e.g., T cell. NK cell) product.

In some embodiments, a subject is not pre-treated with one or more therapies that reduce TREG cells prior to collection of cells for CAR-expressing cell product manufacturing, yet still has a lowered the risk of subject relapse to CAR-expressing cell treatment due to the co-expression of CAR and cytokine-anchor. In some embodiments, a subject is pre-treated with one or more therapies that partially reduce TREG cells prior to collection of cells for CAR-expressing cell product manufacturing, thereby further reducing the risk of subject relapse to CAR-expressing cell treatment.

In some embodiments, a subject is not pre-treated with cyclophosphamide prior to collection of cells for CAR-expressing cell product manufacturing, yet still has lowered risk of subject relapse to CAR-expressing cell treatment due to the co-expression of CAR and cytokine-anchor. In some embodiments, a subject is pre-treated with cyclophosphamide prior to collection of cells for CAR-expressing cell product manufacturing, thereby further reducing the risk of subject relapse to CAR-expressing cell treatment due to the co-expression of CAR and cytokine-anchor. In some embodiments, a subject is not pre-treated with an anti-GITR antibody prior to collection of cells for CAR-expressing cell product manufacturing, yet still has a lowered risk of subject relapse to CAR-expressing cell treatment due to the co-expression of CAR and cytokine-anchor. In some embodiments, a subject is pre-treated with an anti-GITR antibody prior to collection of cells for CAR-expressing cell product manufacturing, thereby further reducing the risk of subject relapse to CAR-expressing cell treatment due to the co-expression of CAR and cytokine-anchor.

In some embodiments, other population of cells can optionally be removed, such as those negatively affect the expansion and/or function of CAR-T cells, e.g., cells expressing CD14, CD11b, CD33, CD15, or other markers expressed by potentially immune suppressive cells.

The methods can further include removing cells from the population which express a tumor antigen, e.g., a tumor antigen that does not comprise CD25, e.g., CD30, CD38, CD123, CD20, CD14 or CD11b, to thereby provide a population of T regulatory depleted, e.g., CD25+ depleted, and tumor antigen depleted cells that are suitable for expression of a CAR, e.g., a CAR19 and cytokine-anchor. In some embodiments, tumor antigen expressing cells are removed simultaneously with the T regulatory, e.g., CD25+ cells. For example, an anti-C25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In some embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory depleted, e.g., CD25+ depleted cells, and check point inhibitor depleted cells, e.g., PD1+, LAG3+ and/or TIM3+ depleted cells. Exemplary check point inhibitors include B7-H1, B&-1, CD160, P1H, 2B4, PD1, TIM3, CEACAM (e.g., CEACAM-1, CEACAM-3 and/or CEACAM-5), LAG3, TIGIT, CTLA-4, BTLA and LAIR1. In some embodiments, check point inhibitor expressing cells are removed simultaneously with the T regulatory, e.g., CD25+ cells. For example, an anti-C25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In some embodiments, the removal of T regulatory cells, e.g., CD25+ cells, and the removal of the check point inhibitor expressing cells is sequential, and can occur, e.g., in either order.

Methods described herein can include a positive selection step. For example, T cells can be isolated by incubation with anti-CD3/anti-CD28 (e.g., 3×28)-conjugated beads, such as DYNABEADS R M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another preferred aspect, the time period is 10 to 24 hours. In one aspect, the incubation time period is 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points.

In some embodiments, a T cell population can be selected that expresses one or more of IFN-γ, TNFα, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in U.S. Patent Publication No.: US20220056116A1, which is hereby incorporated by reference in its entirety.

For isolation of a desired population of cells by positive or negative selection, the concentration of cells and surface (e.g., particles such as beads) can be varied. In some embodiments, it can be desirable to significantly decrease the volume in which beads and cells are mixed together (e.g., increase the concentration of cells), to ensure maximum contact of cells and beads. For example, a concentration of about 10 billion cells/ml, 9 billion/ml, 8 billion/ml, 7 billion/ml, 6 billion/ml, or 5 billion/ml is used. In one aspect, a concentration of 1 billion cells/ml is used. In one aspect, a concentration of cells from 75, 80, 85, 90, 95, or 100 million cells/ml is used. In further aspects, concentrations of 125 or 150 million cells/ml can be used.

Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In some embodiments, it can be desirable to use lower concentrations of cells. By significantly diluting the mixture of T cells and surface (e.g., particles such as beads), interactions between the particles and cells can be minimized. This selects for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10\hat{ }6/ml. In other embodiments, the concentration used is from about 1×10\hat{ }5/ml to 1×10\hat{ }6/ml, and any integer value in between.

In some embodiments, the cells are incubated on a rotator for varying lengths of time at varying speeds at either 2-10° C., or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose. 20% Human Serum Albumin, and 7.5% DMSO or other suitable cell freezing media containing for example, Hespan and PlasmaLyte A, the cells then are frozen to −80° C., at a rate of 10 per minute and stored in the vapor phase of a liquid nitrogen storage tank. Other methods of controlled freezing may be used as well as uncontrolled freezing immediately at −20° C., or in liquid nitrogen.

In some embodiments, cryopreserved cells are thawed and washed as described herein and allowed to rest for one hour at room temperature prior to activation using the methods of the present disclosure.

Also contemplated in the context of the present disclosure is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in immune effector cell therapy for any number of diseases or conditions that would benefit from immune effector cell therapy, such as those described herein. In one aspect a blood sample or an apheresis is taken from a generally healthy subject. In some cases, a blood sample or an apheresis is taken from a generally healthy subject who is at risk of developing a disease, but who has not yet developed a disease, and the cells of interest are isolated and frozen for later use. In some cases, the T cells may be expanded, frozen, and used at a later time. In some cases, samples are collected from a patient shortly after diagnosis of a particular disease as described herein but prior to any treatments. In a further embodiment, the cells are isolated from a blood sample or an apheresis from a subject prior to any number of relevant treatment modalities, including treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cyclophosphamide, fludarabine, cyclosporin. FK506, rapamycin, mycophenolic acid, steroids, FR901228, and irradiation.

In a further embodiment of the present disclosure. T cells are obtained from a patient directly following treatment that leaves the subject with functional T cells. In some cases, following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present disclosure to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells. B cells, dendritic cells, and other cells of the immune system.

In some embodiments, the immune effector cells expressing a CAR molecule, e.g., a CAR19 co-expressed with cytokine-anchor, can be obtained from a subject that has received a low, immune enhancing dose of an mTOR inhibitor. In some embodiments, population of immune effector cells, e.g., T cells or NK cells, which have, or will be engineered to express a CAR, e.g., a CAR19 co-expressed with cytokine-anchor, can be treated ex vivo by contact with an amount of an mTOR inhibitor that increases the number of PD1 negative immune effector cells, e.g., T cells or increases the ratio of PD1 negative immune effector cells, e.g., T cells/NK cells/PD1 positive immune effector cells, e.g., T cells or NK cells.

In some embodiments, a T cell population is diaglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In some embodiments, a T cell population is Ikaros-deficient. Ikaros-deficient cells include cells that do not express Ikaros RNA or protein, or have reduced or inhibited Ikaros activity. Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In some embodiments, the NK cells are obtained from the subject. In another embodiment, the NK cells are an NK cell line, e.g., NK-92 cell line (Conkwest).

### Pharmaceutical Composition

Provided herein are pharmaceutical compositions comprising nucleic acid molecules, cells, immune cells, or systems relating to the cytokines-anchor disclosure. In this regard, the present disclosure provides a pharmaceutical composition comprising any of cytokine-anchor materials described herein and a pharmaceutically acceptable excipient or carrier. In some embodiments, the pharmaceutical composition comprises a cell described herein, for example an engineered immune cell expressing CAR and cytokine-anchor, and a pharmaceutically acceptable excipient or carrier.

In some embodiments, the carrier is a pharmaceutically acceptable carrier. The pharmaceutically acceptable carrier can be any of those conventionally used for the administration of cells. The pharmaceutically acceptable carriers are well-known to those skilled in the art. The pharmaceutically acceptable carriers can be one which has no detrimental side effects or toxicity under the conditions of use.

The carrier can be determined in part by the particular method used to administer the particular cytokine-anchor materials. There are a variety of suitable formulations of the pharmaceutical composition of the present disclosure. Suitable formulations may include any of those for parenteral, subcutaneous, intravenous, intramuscular, intraarterial, intrathecal, intratumoral, or intraperitoneal administration. More than one route can be used to administer the inventive IL-21/15 material, and in certain instances, a particular route can provide a more immediate and more effective response than another route.

In some embodiments, one or more CAR-expressing cells as disclosed herein can be administered or delivered to the subject via a biopolymer scaffold, e.g., a biopolymer implant. Biopolymer scaffolds can support or enhance the delivery, expansion, and/or dispersion of the CAR-expressing cells described herein. A biopolymer scaffold comprises a biocompatible (e.g., does not substantially induce an inflammatory or immune response) and/or a biodegradable polymer that can be naturally occurring or synthetic.

Examples of suitable biopolymers include agar, agarose, alginate, alginate/calcium phosphate cement (CPC), beta-galactosidase (β-GAL), (1,2,3,4,6-pentaacetyl a-D-galactose), cellulose, chitin, chitosan, collagen, elastin, gelatin, hyaluronic acid collagen, hydroxyapatite, poly(3-hydroxy butyrate-co-3-hydroxy-hexanoate) (PHBHHx), poly(lactide), poly(caprolactone) (PCL), poly(lactide-co-glycolide) (PLG), polyethylene oxide (PEO), poly(lactic-co-glycolic acid) (PLGA), polypropylene oxide (PPO), polyvinyl alcohol) (PVA), silk, soy protein, and soy protein isolate, alone or in combination with any other polymer composition, in any concentration and in any ratio. The biopolymer can be augmented or modified with adhesion- or migration-promoting molecules, e.g., collagen-mimetic peptides that bind to the collagen receptor of lymphocytes, and/or stimulatory molecules to enhance the delivery, expansion, or function, e.g., anti-cancer activity, of the cells to be delivered. The biopolymer scaffold can be an injectable, e.g., a gel or a semi-solid, or a solid composition.

In some embodiments, cells expressing CAR and cytokine-anchor described herein can be seeded onto the biopolymer scaffold prior to delivery to the subject. In embodiments, the biopolymer scaffold further comprises one or more additional therapeutic agents described herein (e.g., another CAR-expressing cell, an antibody, or a small molecule) or agents that enhance the activity of cells expressing CAR, e.g., incorporated or conjugated to the biopolymers of the scaffold. In embodiments, the biopolymer scaffold is injected, e.g., intratumorally, or surgically implanted at the tumor or within a proximity of the tumor sufficient to mediate an anti-tumor effect. Additional examples of biopolymer compositions and methods for their delivery are described in Stephan et al., Nature Biotechnology. 2015, 33:97-101; and U.S. Patent Publication No. US20210015863A1, each of which is hereby incorporated herein by reference in its entirety.

In some embodiments, the cells are administered by injection, e.g., intravenously. A suitable pharmaceutically acceptable carrier for the cells for injection may include any isotonic carrier such as, for example, normal saline (about 0.90% w/v of NaCl in water, about 300 mOsm/L NaCl in water, or about 9.0 g NaCl per liter of water). NORMOSOL R electrolyte solution (Abbott, Chicago, Ill.), PLASMA-LYTE A (Baxter, Deerfield, Ill.), about 5% dextrose in water, or Ringer's lactate. In some embodiments, the pharmaceutically acceptable carrier is supplemented with human serum albumen.

In some embodiments, the pharmaceutical compositions comprise a cell or a plurality of cells expressing CAR and cytokine-anchor, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextran, mannitol; proteins; polypeptides or amino acids such as glycine; antioxidants; chelating agents such as EDTA or glutathione; adjuvants (e.g., aluminum hydroxide); and preservatives. Compositions of the present disclosure are in one embodiment formulated for intravenous administration.

Pharmaceutical compositions of the present disclosure may be administered in a manner appropriate to the disease to be treated (or prevented). The quantity and frequency of administration will be determined by such factors as the condition of the patient, and the type and severity of the patient's disease, although appropriate dosages may be determined by clinical trials.

In some embodiments, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In some embodiments, the bacterium is at least one selected from the group consisting of Alcaligenes faecalis, Candida albicans, Escherichia coli, Haemophilus influenzae, Neisseria meningitides, Pseudomonas aeruginosa, Staphylococcus aureus, Streptococcus pneumonia, and Streptococcus pyogenes group A.

### Kit

In some aspects, provided herein are kits comprising the unit doses containing the nucleic acid molecules, vectors, systems, cells, or pharmaceutical compositions of the cytokine-anchor materials and instructions for use. In some embodiments, the kit comprises the cells (e.g., engineered immune cells) expressing the nucleic acid molecules described in the cytokine-anchor materials, for example, at least one CAR19, and/or at least one cytokine linked to an anchoring structure; and an information material containing instructions for administering a dosage of the immune cell, the cell, or a dosage form of the pharmaceutical composition to a subject.

The kit can further comprise one or more unit doses containing one or more additional reagents, such as an immunosuppressive reagent, or one or more additional therapeutic agent, such as antibodies. Kits typically include a label indicating the intended use of the contents of the kit. The term label includes any writing, or recorded material supplied on or with the kit, or which otherwise accompanies the kit.

A kit of the present disclosure may also include diagnostic agents and/or other therapeutic agents. In some cases, a kit includes cells or pharmaceutical compositions of the present disclosure and a diagnostic agent that may be used in a diagnostic method for diagnosing the state or existence of a disease, condition or disorder in a subject.

### Method of Treatment, Administration, and Uses

In another aspect, provided herein are methods of using the cytokine-anchor materials disclosed herein to treat a subject and methods of administering the same to a subject. The subject can have a certain disease or a condition in need of treatment provided by the present disclosure. The disease or condition can include, e.g., reducing or ameliorating, a hyperproliferative condition or disorder, for example a cancer. The disease or condition can include solid tumor, a soft tissue tumor, or a metastatic lesion. As used herein, the term “cancer” is meant to include all types of cancerous growths or oncogenic processes, metastatic tissues or malignantly transformed cells, tissues, or organs, irrespective of histopathologic type or stage of invasiveness.

Examples of solid tumors include malignancies, e.g., sarcomas, adenocarcinomas, and carcinomas, of the various organ systems, such as those affecting liver, lung, breast, lymphoid, gastrointestinal (e.g., colon), genitourinary tract (e.g., renal, urothelial cells), prostate and pharynx. Adenocarcinomas include malignancies such as most colon cancers, rectal cancer, renal-cell carcinoma, liver cancer, non-small cell carcinoma of the lung, cancer of the small intestine and cancer of the esophagus. In some embodiments, the cancer is a melanoma, e.g., an advanced stage melanoma. Metastatic lesions of the aforementioned cancers can also be treated or prevented using the methods and compositions of the invention. Examples of other cancers that can be treated include bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva. Hodgkin Disease, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma. Kaposi's sarcoma, epidermoid cancer, squamous cell cancer, T-cell lymphoma, environmentally induced cancers including those induced by asbestos, and combinations of said cancers.

Exemplary cancers whose growth can be inhibited include cancers typically responsive to immunotherapy. Examples of cancers for treatment include melanoma (e.g., metastatic malignant melanoma), renal cancer (e.g., clear cell carcinoma), prostate cancer (e.g., hormone refractory prostate adenocarcinoma), breast cancer, colon cancer and lung cancer (e.g., non-small cell lung cancer). Additionally, refractory or recurrent malignancies can be treated using the molecules described herein.

In some embodiments, the method treats cancer in a subject. In some embodiments, the method treats a solid tumor. In some embodiments, the cancer comprises leukemia. In some embodiments, the cancer comprises melanoma. In some embodiments, the cancer comprises lymphoma. In some embodiments, the cancer comprises adrenal gland cancer, bladder cancer, bone cancer, brain tumor, breast cancer, cervical cancer, colorectal cancer, esophageal cancer, fallopian tube cancer, gastrointestinal cancer, glioma, glioblastoma, head and neck cancer, hematopoietic malignancy, leukemia, liver cancer, lung cancer, lymphoma, myeloma, nasal cancer, nasopharyngeal cancer, oral cancer, oropharyngeal cancer, ovarian cancer, pancreatic cancer, prostate cancer, sarcoma, stomach cancer, squamous cell lung cancer, testicular cancer, thyroid cancer, uterine cancer, or any combination thereof.

In some embodiments, the method comprises administering to a subject a cell expressing the CAR and/or cytokine-anchor or the pharmaceutical composition comprising the same. In some cases, the method comprises administering to a subject an immunologically effective amount of the immune cell expressing the CAR and/or cytokine-anchor or the pharmaceutical composition comprising the same. In some cases, the method comprises administering to a subject a therapeutically effective amount of the immune cell expressing the CAR and/or cytokine-anchor or the pharmaceutical composition comprising the same. In some cases, the cell is an immune cell, for example, an engineered immune cell. In some cases, the immune cell or the cell is allogeneic to the subject. In some cases, the immune cell or the cell is autologous to the subject. In some cases, the method further comprises obtaining a population of immune cells, and engineering the population of immune cells or a progeny thereof to produce the engineered immune cell.

When “an immunologically effective amount,” “an anti-tumor effective amount,” “a tumor-inhibiting effective amount,” or “therapeutically effective amount” is indicated, the precise amount of the compositions of the present disclosure to be administered can be determined by a physician with consideration of individual differences in age, weight, tumor size, extent of infection or metastasis, and condition of the patient (subject). It can generally be stated that a pharmaceutical composition comprising the immune effector cells (e.g., T cells, NK cells) described herein may be administered at a dosage of 104 to 109 cells/kg body weight, in some instances 105 to 106 cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J, of Med. 319:1676, 1988).

In some cases, it can be desirable to administer activated immune effector cells (e.g., T cells, NK cells) to a subject and then subsequently redraw blood (or have an apheresis performed), activate immune effector cells (e.g., T cells, NK cells) therefrom according to the present disclosure, and reinfuse the patient with these activated and expanded immune effector cells (e.g., T cells, NK cells). This process can be carried out multiple times every few weeks. In some cases, immune effector cells (e.g., T cells, NK cells) can be activated from blood draws of from 10 cc to 400 cc. In some cases, immune effector cells (e.g., T cells, NK cells) are activated from blood draws of 20 cc, 30 cc, 40 cc, 50 cc, 60 cc, 70 cc, 80 cc, 90 cc, or 100 cc.

The administration of the pharmaceutical compositions to a subject can be carried out in any convenient manner, including by aerosol inhalation, injection, ingestion, transfusion, implantation or transplantation. The pharmaceutical compositions described herein may be administered to a patient trans arterially, subcutaneously, intradermally, intratumorally, intranodally, intramedullary, intramuscularly, by intravenous (i.v.) injection, or intraperitoneally. In some embodiments, the T cell compositions of the present disclosure are administered to a patient by intradermal or subcutaneous injection. In some embodiments, the T cell compositions of the present disclosure are administered by i.v. injection. The compositions of immune effector cells (e.g., T cells, NK cells) may be injected directly into a tumor, lymph node, or site of infection.

In some embodiments, a subject is not pre-treated with one or more therapies that are administered solely for the purpose of reducing lymphocytes of the subject prior to administering a cell expressing the CAR and/or cytokine-anchor described herein or the pharmaceutical composition comprising the same. In some embodiments, a subject is not pre-treated with one or more therapies that partially depletes T cells of the subject prior to administering a cell expressing the CAR and/or cytokine-anchor described herein or the pharmaceutical composition comprising the same. In some embodiments, a subject does not receive or has not received lymphodepletion prior to administering a cell expressing the CAR and/or cytokine-anchor described herein or the pharmaceutical composition comprising the same. In some embodiments, a subject does not receive or has not received preconditioning that involves a short course of chemotherapies (e.g., cyclophosphamide (Cy) alone or Cy combined with fludarabine) solely for the purpose of depleting lymphocytes of the subject prior to administering a cell expressing the CAR and/or cytokine-anchor described herein or the pharmaceutical composition comprising the same. In some embodiments, lymphodepletion process is not used throughout a course of treatment that administers to a subject in need thereof a cell expressing the CAR and/or cytokine-anchor described herein or the pharmaceutical composition comprising the same.

In some cases, subjects can undergo leukapheresis, wherein leukocytes are collected, enriched, or depleted ex vivo to select and/or isolate the cells of interest, e.g., T cells. These T cell isolates may be expanded by methods known in the art and treated such that one or more CAR constructs of the present disclosure may be introduced, thereby creating a CAR T cell of the present disclosure. Subjects in need thereof may subsequently undergo standard treatment with high dose chemotherapy followed by peripheral blood stem cell transplantation. In certain embodiments, following or concurrent with the transplant, subjects receive an infusion of the expanded CAR T cells of the present disclosure. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

In one aspect, provided herein are uses of the cells or the pharmaceutical composition of the cytokine-anchor materials, in the manufacture of a medicament to treat a subject. In some cases, the cell is an immune cell, for example, an engineered immune cell. In some cases, the immune cell or the cell is allogeneic to the subject. In some cases, the immune cell or the cell is autologous to the subject. In some cases, the immune cell or the cell treats a cancer described herein in the subject. In some embodiments, the cell expresses a CAR, such as CAR19, and/or at least one cytokine linked to an anchoring structure. In some embodiments, the cytokine is anchored to the cell membrane.

In some cases, the nucleic acid molecules described herein, such as vectors comprising CAR and/or cytokine-anchor, is introduced into immune effector cells (e.g., T cells, NK cells), e.g., using in vitro transcription, and the subject (e.g., human) receives an initial administration of such engineered immune effector cells (e.g., T cells, NK cells) of the present disclosure, and one or more subsequent administrations of the engineered immune effector cells (e.g., T cells, NK cells) of the present disclosure, wherein the one or more subsequent administrations are administered less than 15 days, e.g., 14, 13, 12, 11, 10, 9, 8, 7, 6, 5, 4, 3, or 2 days after the previous administration. In some embodiments, more than one administration of the engineered immune effector cells (e.g., T cells, NK cells) of the present disclosure are administered to the subject (e.g., human) per week, e.g., 2, 3, or 4 administrations of the engineered immune effector cells (e.g., T cells, NK cells) of the present disclosure are administered per week. In some embodiments, the subject (e.g., human subject) receives more than one administration of the engineered immune effector cells (e.g., T cells, NK cells) per week (e.g., 2, 3 or 4 administrations per week) (also referred to herein as a cycle), followed by a week of no engineered immune effector cells (e.g., T cells, NK cells) administrations, and then one or more additional administration of the engineered immune effector cells (e.g., T cells, NK cells) (e.g., more than one administration of the engineered immune effector cells (e.g., T cells, NK cells) per week) is administered to the subject. In another embodiment, the subject (e.g., human subject) receives more than one cycle of engineered immune effector cells (e.g., T cells, NK cells), and the time between each cycle is less than 10, 9, 8, 7, 6, 5, 4, or 3 days. In some embodiments, the engineered immune effector cells (e.g., T cells, NK cells) are administered every other day for 3 administrations per week. In some embodiments, the engineered immune effector cells (e.g., T cells, NK cells) of the present disclosure are administered for at least two, three, four, five, six, seven, eight or more weeks.

In some cases, cells expressing CAR and/or cytokine-anchor of the present disclosures are generated using lentiviral viral vectors, such as lentivirus. Cells, e.g., engineered immune cells expressing CAR and/or cytokine-anchor, generated that way will have stable expression of CAR and cytokine-anchor.

In some cases, cells expressing CAR and/or cytokine-anchor of the present disclosures are generated using a viral vector such as a gammaretroviral vector, e.g., a gammaretroviral vector described herein. Cells, e.g., engineered immune cells expressing CAR and/or cytokine-anchor, generated using these vectors will have stable expression of CAR and cytokine-anchor.

In some cases, engineered immune cells expressing CAR and/or cytokine-anchor transiently express vectors for 4, 5, 6, 7, 8, 9, 10, 11, 12, 13, 14, 15 days after transduction. Transient expression of CAR and/or cytokine-anchor can be affected by RNA vector delivery. In some cases, the CAR and/or cytokine-anchor RNA is transduced into the T cell by electroporation.

### Cell Proliferation Effect

In some cases, the cells of the cytokine-anchor materials described herein exhibit improved ability to proliferate. In some cases, the cells are immune cells, for example, engineered immune cells. In some cases, the cells express a CAR, for example CAR19, and/or at least one cytokine and anchoring structure. In some cases, the at least one cytokine is anchored on the cell membrane via the anchoring structure. The improved ability to proliferate can refer to a population of said immune cells proliferates for a longer period of time relative to a population of comparable immune cells that does not comprise a component described in the cytokine-anchor materials. In some embodiments, a population of said immune cells proliferates for a longer period of time relative to a population of comparable immune cells that does not comprise nucleic acid sequences encoding a polypeptide described in the cytokine-anchor materials.

In some embodiments, the duration of proliferation of the cell comprising the cytokine-anchor materials described herein is at least about 1% to at least about 95% longer than a cell that does not comprise the cytokine-anchor materials described herein. In some embodiments, the duration of proliferation of the cell comprising the cytokine-anchor materials described herein is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% longer than a cell that does not comprise the cytokine-anchor materials described herein.

In some embodiments, the duration of proliferation of the cell comprising cytokine-anchor materials described herein is about 1% to about 95% longer than a cell that does not comprise the cytokine-anchor materials described herein. In some embodiments, the duration of proliferation of the cell comprising the cytokine-anchor materials described herein is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95% longer than a cell that does not comprise the cytokine-anchor materials described herein.

In some embodiments, the duration of proliferation of the cell comprising the cytokine-anchor materials described herein is at least about 1% to at least about 95% longer than a reference cell that comprising secreted cytokines (i.e., not anchored to the cell membrane) that is the same as the cytokines of the cytokine-anchor materials described herein, wherein the cell and the reference cell comprise CAR19. In some embodiments, the duration of proliferation of the cell comprising the cytokine-anchor materials described herein is at least about 10%, 20%, 30% or 40% longer than the reference cell. In some cases, the cell comprising the cytokine-anchor materials described herein is a cell comprising CAR19 and TeIL-10. In some cases, the reference cell comprises CAR19 and secreted IL-10.

In some embodiments, the duration of improved proliferation is from about 1 hour to about 72 hours. In some embodiments, the duration of improved proliferation is from about 24 hours to about 72 hours. In some embodiments, the duration of improved proliferation is from about 1 day to about 10) days. In some embodiments, the duration of improved proliferation is from about 5 days to about 20 days. In some embodiments, the duration of improved proliferation is from about 1 day to about 1 year. In some embodiments, the duration of improved proliferation is from about 1 month to about 12 months.

In some embodiments, the duration of proliferation is about 1 hour 1, about 2 hours, about 3 hours, about 4 hours, about 5 hours, about 6 hours, about 7 hours, about 8 hours, about 9 hours, about 10) hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30) hours, about 31 hours, about 32 hours, about 33 hours, about 34 hours, about 35 hours, about 36 hours, about 37 hours, about 38 hours, about 39 hours, about 40) hours, about 41 hours, about 42 hours, about 43 hours, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70) hours, about 71 hours, or about 72 hours.

In some embodiments, the duration of proliferation is about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, or about 25 years.

Cell proliferation can be measured by any suitable method, such as quantification of expression levels (i.e., mRNA, protein) of any one or more suitable cell proliferation markers. Exemplary methods can include immunofluorescent imaging, immunohistochemistry, flow cytometry, qPCR, or next generation sequencing. Cell proliferation markers can include Cyclin D1, Cyclin E, Cyclin A, Cyclin B, CDK2, CDK4/6, Cdk1, or pRb. In some embodiments, cell proliferation can be determined by Edu pulse-chase experiment or Brdu pulse-chase experiment.

A “comparable immune cell” or “a population of comparable immune cells” used herein refers to a reference immune cell or a reference population of immune cells that are substantially the same as the immune cell or the population immune cells except for lacking specific features (e.g., does not express the membrane-anchored cytokines described herein) as described in each embodiment.

In some embodiments, for example, an embodiment for the single cytokine-anchor materials, said population of comparable immune cells (a) does not comprise the exogenous nucleic acid sequence encoding the polypeptide comprising the cytokine peptide and the non-peptide anchor attachment signal; (b) does not comprise the nucleic acid sequence encoding the polypeptide comprising the signal peptide, the cytokine peptide and the non-peptide anchor attachment signal; (c) does not comprise the nucleic acid sequence encoding the polypeptide comprising the cytokine peptide and the peptide anchor; or (d) does not comprise protein comprising the cytokine peptide and (i) the non-peptide anchor or (ii) the peptide anchor. In some embodiments, said population of comparable immune cells expresses a CAR, such as a CAR19.

In some embodiments, for example, an embodiment for the two cytokine-anchor materials, said population of comparable immune cells (a) does not comprise the first nucleic acid sequence encodes the first polypeptide comprising the first cytokine peptide and (i) the first non-peptide anchor attachment signal or (ii) the first peptide anchor, and/or does not comprise the second nucleic acid sequence encodes the second polypeptide comprising the second cytokine peptide and (i) the second non-peptide anchor attachment signal or (ii) the second peptide anchor; or (b) does not comprise the first protein comprising the first cytokine peptide and (i) the first non-peptide anchor or (ii) the first peptide anchor, and/or does not comprise the second protein comprising the second cytokine peptide and (i) the second non-peptide anchor or (ii) the second peptide anchor. In some embodiments, said population of comparable immune cells expresses a CAR, such as a CAR19. In some cases, the population of immune cells comprises CAR19, a first polypeptide comprising the first cytokine peptide that is anti-inflammatory (e.g., IL-4 or IL-10) and (i) the first non-peptide anchor attachment signal or (ii) the first peptide anchor and a second nucleic acid sequence encodes the second polypeptide comprising the second cytokine peptide that is pro-inflammatory (e.g., IL-2, IL-7, IL-9, IL-12, IL-12p40, IL-15, IL-18, IL-21, IL-23, IL-36γ, IL-23p19, or IL-1α) and (i) the second non-peptide anchor attachment signal or (ii) the second peptide anchor, and the population of comparable immune cells comprises CAR19 and TeIL-4 and does not express TeIL-15. In some cases, the population of immune cells comprises CAR19, TeIL-4 and TeIL-15, and the population of comparable immune cells comprises CAR19 and TeIL-4 and does not express TeIL-15.

In some embodiments, for example, an embodiment for the three cytokine-anchor materials, said population of comparable immune cells (a) does not comprise all of: (1) the first nucleic acid sequence encodes the first polypeptide comprising the first cytokine peptide and (i) a first non-peptide anchor attachment signal or (ii) the first peptide anchor; (2) the second nucleic acid sequence encodes the second polypeptide comprising the second cytokine peptide and (i) the second non-peptide anchor attachment signal or (ii) the second peptide anchor; and (3) the third nucleic acid sequence encodes the third polypeptide comprising the third cytokine peptide and (i) the third non-peptide anchor attachment signal or (ii) the third peptide anchor; or (b) does not comprise all of: (1) the first protein comprising the first cytokine peptide and (i) the first non-peptide anchor or (ii) the first peptide anchor; (2) the second protein comprising the second cytokine peptide and (i) the second non-peptide anchor or (ii) the second peptide anchor; and (3) the third protein comprising the third cytokine peptide and (i) the third non-peptide anchor or (ii) the third peptide anchor. In some embodiments, said population of comparable immune cells expresses a CAR, such as a CAR19.

In some embodiments, proliferation of said population of said immune cells is measured by an in vitro immune cell proliferation assay described in Examples A-E. In some embodiments, proliferation of said population of said immune cells is measured in vitro or in vivo. In some embodiments, proliferation of said population of said immune cells lasts for a period of time at least about 5%, 10%, 20%, 30%, 40%, 50%, 100%, 2 times, 3 times, 5 times, 10 times or longer than said population of said comparable immune cells. In some embodiments, proliferation of said population of said immune cells lasts for a period of time at least about 5% or longer than said population of said comparable immune cells. In some embodiments, proliferation of said population of said immune cells lasts for a period of time at least about 10% or longer than said population of said comparable immune cells. In some embodiments, proliferation of said population of said immune cells lasts for a period of time at least about 20% or longer than said population of said comparable immune cells. In some embodiments, proliferation of said population of said immune cells lasts for a period of time at least about 30% or longer than said population of said comparable immune cells. In some embodiments, proliferation of said population of said immune cells lasts for a period of time at least about 40% or longer than said population of said comparable immune cells. In some embodiments, proliferation of said population of said immune cells lasts for a period of time at least about 50% or longer than said population of said comparable immune cells. In some embodiments, proliferation of said population of said immune cells lasts for a period of time at least about 60% or longer than said population of said comparable immune cells. In some embodiments, proliferation of said population of said immune cells lasts for a period of time at least about 70% or longer than said population of said comparable immune cells. In some embodiments, proliferation of said population of said immune cells lasts for a period of time at least about 80% or longer than said population of said comparable immune cells. In some embodiments, proliferation of said population of said immune cells lasts for a period of time at least about 90% or longer than said population of said comparable immune cells. In some embodiments, proliferation of said population of said immune cells lasts for a period of time at least about 100% or longer than said population of said comparable immune cells. In some embodiments, proliferation of said population of said immune cells lasts for a period of time at least about 2 times or longer than said population of said comparable immune cells. In some embodiments, proliferation of said population of said immune cells lasts for a period of time at least about 3 times or longer than said population of said comparable immune cells. In some embodiments, proliferation of said population of said immune cells lasts for a period of time at least about 4 times or longer than said population of said comparable immune cells. In some embodiments, proliferation of said population of said immune cells lasts for a period of time at least about 5 times or longer than said population of said comparable immune cells. In some embodiments, proliferation of said population of said immune cells lasts for a period of time at least about 6 times or longer than said population of said comparable immune cells. In some embodiments, proliferation of said population of said immune cells lasts for a period of time at least about 7 times or longer than said population of said comparable immune cells. In some embodiments, proliferation of said population of said immune cells lasts for a period of time at least about 8 times or longer than said population of said comparable immune cells. In some embodiments, proliferation of said population of said immune cells lasts for a period of time at least about 9 times or longer than said population of said comparable immune cells. In some embodiments, proliferation of said population of said immune cells lasts for a period of time at least about 10 times or longer than said population of said comparable immune cells.

### Cell Cytotoxicity

In some cases, the cells of the cytokine-anchor materials described herein exhibit improved or continuous cell cytotoxicity. In some cases, the cells are immune cells, for example, engineered immune cells. In some cases, the cells express a CAR, for example CAR19, and/or at least one cytokine and anchoring structure. In some cases, the at least one cytokine is anchored on the cell membrane via the anchoring structure. The improved cell cytotoxicity can refer to increase in cytotoxicity of said immune cell relative to a comparable immune cell that does not comprise a component described in the cytokine-anchor materials. In some embodiments, cytotoxicity of said immune cell is increased relative to a comparable immune cell that does not comprise nucleic acid sequences encoding a polypeptide described in the cytokine-anchor materials.

Continuous cell cytotoxicity can refer to prolonged duration of cytotoxicity of said immune cell relative to a comparable immune cell that does not comprise a component described in the cytokine-anchor materials. In some embodiments, cytotoxicity of said immune cell is prolonged relative to a comparable immune cell that does not comprise nucleic acid sequences encoding a polypeptide described in the cytokine-anchor materials.

In some embodiments, cytotoxicity of a cell is the cell's ability to induce tumor lysis. In some embodiments, cytotoxicity is determined by tumor lysis. Tumor lysis can be determined by measuring the change in number of live tumor cells over a period of time. Tumor lysis can be determined by measuring the change in number of dead tumor cells over a period of time. In some embodiments, cytotoxicity is expressed as percentage of tumor lysis over a period of time.

In some embodiments, the cells, compositions, methods, and systems described herein promotes continuous cytotoxicity of an immune cell. In some embodiments, the cells, compositions, methods, and systems described herein can promote long-term cytotoxicity of an immune cell.

In some embodiments, the duration of cytotoxicity of the cell comprising the cytokine-anchor materials described herein is at least about 1% to at least about 95% longer than a cell that does not comprise the cytokine-anchor materials described herein. In some embodiments, the duration of cytotoxicity of the cell comprising the cytokine-anchor materials described herein is at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, or at least 95% longer than a cell that does not comprise the cytokine-anchor materials described herein.

In some embodiments, the duration of cytotoxicity of the cell comprising the cytokine-anchor materials described herein is about 1% to about 95% longer than a cell that does not comprise the cytokine-anchor materials described herein. In some embodiments, the duration of cytotoxicity of the cell comprising the cytokine-anchor materials described herein is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95% longer than a cell that does not comprise the cytokine-anchor materials described herein.

In some embodiments, the duration of cytotoxicity of the cell comprising the cytokine-anchor materials described herein is at least about 1% to at least about 95% longer than a reference cell that comprising secreted cytokines (i.e., not anchored to the cell membrane) that is the same as the cytokines of the cytokine-anchor materials described herein, wherein the cell and the reference cell comprise CAR19. In some embodiments, the duration of cytotoxicity of the cell comprising the cytokine-anchor materials described herein is at least about 10%, 20%, or 30% longer than the reference cell. In some cases, the cell comprising the cytokine-anchor materials described herein is a cell comprising CAR19 and TeIL-10. In some cases, the reference cell comprises CAR19 and secreted IL-10.

In some embodiments, the duration of improved cytotoxicity is from about 1 hour to about 72 hours. In some embodiments, the duration of improved cytotoxicity is from about 24 hours to about 72 hours. In some embodiments, the duration of improved cytotoxicity is from about 1 day to about 10) days. In some embodiments, the duration of improved cytotoxicity is from about 5 days to about 20 days. In some embodiments, the duration of improved cytotoxicity is from about 1 day to about 1 year. In some embodiments, the duration of improved cytotoxicity is from about 1 month to about 12 months.

In some embodiments, the duration of improved cytotoxicity is about 1 hour 1, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30) hours, about 31 hours, about 32 hours, about 33 hours, about 34 hours, about 35 hours, about 36 hours, about 37 hours, about 38 hours, about 39 hours, about 40) hours, about 41 hours, about 42 hours, about 43 hours, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60) hours, about 61 hours, about 62 hours, about 63 hours, about 64 hours, about 65 hours, about 66 hours, about 67 hours, about 68 hours, about 69 hours, about 70 hours, about 71 hours, or about 72 hours.

In some embodiments, the duration of improved cytotoxicity is about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10) months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, or about 25 years.

In some embodiments, continuous or long-term cytotoxicity can be the reduction of T cell exhaustion. T cell exhaustion is a response of T cells to chronic antigen stimulation. In some embodiments, an exhausted T cell fails to self-renew or proliferate. In some embodiments, an exhausted T cell has a reduced capacity to secrete cytokines. In some embodiments, an exhausted T cell fails to illicit anti-tumor immune response.

In some embodiments, the cells, compositions, methods, and systems described herein recuses T cell exhaustion.

Cytotoxicity can be measured by any suitable methods. In some embodiments, cytotoxicity is determined by cell viability assays, apoptosis assays, or cell proliferation assays.

In some embodiments, for example, an embodiment for the single cytokine-anchor materials, said population of comparable immune cells (a) does not comprise the exogenous nucleic acid sequence encoding the polypeptide comprising the cytokine peptide and the non-peptide anchor attachment signal; (b) does not comprise the nucleic acid sequence encoding the polypeptide comprising the signal peptide, the cytokine peptide and the non-peptide anchor attachment signal; (c) does not comprise the nucleic acid sequence encoding the polypeptide comprising the cytokine peptide and the peptide anchor; or (d) does not comprise protein comprising the cytokine peptide and (i) the non-peptide anchor or (ii) the peptide anchor. In some embodiments, said population of comparable immune cells expresses a CAR, such as a CAR19.

In some embodiments, for example, an embodiment for the two cytokine-anchor materials, said population of comparable immune cells (a) does not comprise both of: (1) the first nucleic acid sequence encodes the first polypeptide comprising the first cytokine peptide and (i) the first non-peptide anchor attachment signal or (ii) the first peptide anchor; and (2) the second nucleic acid sequence encodes the second polypeptide comprising the second cytokine peptide and (i) the second non-peptide anchor attachment signal or (ii) the second peptide anchor; or (b) does not comprise both of: (1) the first protein comprising the first cytokine peptide and (i) the first non-peptide anchor or (ii) the first peptide anchor; and (2) the second protein comprising the second cytokine peptide and (i) the second non-peptide anchor or (ii) the second peptide anchor. In some embodiments, said population of comparable immune cells expresses a CAR, such as a CAR19.

In some embodiments, for example, an embodiment for the three cytokine-anchor materials, said population of comparable immune cells (a) does not comprise all of: (1) the first nucleic acid sequence encodes the first polypeptide comprising the first cytokine peptide and (i) a first non-peptide anchor attachment signal or (ii) the first peptide anchor; (2) the second nucleic acid sequence encodes the second polypeptide comprising the second cytokine peptide and (i) the second non-peptide anchor attachment signal or (ii) the second peptide anchor; and (3) the third nucleic acid sequence encodes the third polypeptide comprising the third cytokine peptide and (i) the third non-peptide anchor attachment signal or (ii) the third peptide anchor; or (b) does not comprise all of: (1) the first protein comprising the first cytokine peptide and (i) the first non-peptide anchor or (ii) the first peptide anchor; (2) the second protein comprising the second cytokine peptide and (i) the second non-peptide anchor or (ii) the second peptide anchor; and (3) the third protein comprising the third cytokine peptide and (i) the third non-peptide anchor or (ii) the third peptide anchor. In some embodiments, said population of comparable immune cells expresses a CAR, such as a CAR19.

In some embodiments, increase in cytotoxicity of said immune cell is measured by an in vitro cytotoxicity assay described in Examples A-D. In some embodiments, said increase in cytotoxicity of said immune cell is at least about 5%, 10%, 20%, 30%, 40%, 50% or more. In some embodiments, said increase in cytotoxicity of said immune cell is at least about 5% or more. In some embodiments, said increase in cytotoxicity of said immune cell is at least about 10% or more. In some embodiments, said increase in cytotoxicity of said immune cell is at least about 20% or more. In some embodiments, said increase in cytotoxicity of said immune cell is at least about 30% or more. In some embodiments, said increase in cytotoxicity of said immune cell is at least about 40% or more. In some embodiments, said increase in cytotoxicity of said immune cell is at least about 50% or more. In some embodiments, said increase in cytotoxicity of said immune cell is at least about 60% or more. In some embodiments, said increase in cytotoxicity of said immune cell is at least about 70% or more. In some embodiments, said increase in cytotoxicity of said immune cell is at least about 80% or more. In some embodiments, said increase in cytotoxicity of said immune cell is at least about 90% or more. In some embodiments, said increase in cytotoxicity of said immune cell is at least about 100% or more.

### T Cell Activation

In some cases, the cells of the cytokine-anchor materials described herein exhibit improved or continuous T cell activation. In some cases, the cells are immune cells, for example, engineered immune cells. In some cases, the cells express a CAR, for example CAR19, and/or at least one cytokine and anchoring structure. In some cases, the at least one cytokine is anchored on the cell membrane via the anchoring structure. The improved cell cytotoxicity can refer to increase in cytotoxicity of said immune cell relative to a comparable immune cell that does not comprise a component described in the cytokine-anchor materials. In some embodiments, activation of said immune cell is increased relative to a comparable immune cell that does not comprise nucleic acid sequences encoding a polypeptide described in the cytokine-anchor materials. In some embodiments, activation of said immune cell is prolonged relative to a comparable immune cell that does not comprise nucleic acid sequences encoding a polypeptide described in the cytokine-anchor materials.

The continuous activation can refer to prolonged duration of activation of said immune cell relative to a comparable immune cell that does not comprise a component described in the cytokine-anchor materials.

Immune cell activation can be determined by any suitable assays, including T cell activation assays such as measurements of activation markers and TCRαβ-KO

### Combination Therapies

A nucleic acid molecule, a cell, a system, a pharmaceutical composition, a kit described herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A cell expressing any components of the cytokine-anchor materials, e.g., CAR19 and cytokine-anchor and the at least one additional therapeutic agent can be administered simultaneously, in the same or in separate compositions, or sequentially. For sequential administration, the cell expressing any components of the cytokine-anchor materials, e.g., CAR19 and cytokine-anchor can be administered first, and the additional agent can be administered second, or the order of administration can be reversed.

The CAR therapy and/or other therapeutic agents, procedures or modalities can be administered during periods of active disorder, or during a period of remission or less active disease. The CAR therapy can be administered before the other treatment, concurrently with the treatment, post-treatment, or during remission of the disorder.

When administered in combination, the CAR therapy and the additional agent (e.g., second or third agent), or all, can be administered in an amount or dose that is higher, lower or the same than the amount or dosage of each agent used individually, e.g., as a monotherapy. In certain embodiments, the administered amount or dosage of the CAR therapy, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the CAR therapy, the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

In other cases, a cell expressing any components of the cytokine-anchor materials, e.g., CAR19 and cytokine-anchor may be used in a treatment regimen in combination with surgery, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cyclophosphamide, fludarabine, cyclosporin. FK506, rapamycin, mycophenolic acid, steroids. FR901228, cytokines, and irradiation, peptide vaccine, such as that described in Izumoto et al. 2008 J Neurosurg 108:963-971, which is hereby incorporated herein by reference in its entirety.

In some cases, a cell expressing any components of the cytokine-anchor materials, e.g., CAR19 and cytokine-anchor can be used in combination with a chemotherapeutic agent, an antibody, an alkylating agent, a mTOR inhibitor, a corticosteroid, or any other suitable therapeutic agents.

### EXAMPLES

The following examples are provided to further illustrate some embodiments of the present disclosure, but are not intended to limit the scope of the disclosure: it will be understood by their exemplary nature that other procedures, methodologies, or techniques known to those skilled in the art may alternatively be used.

### Example A. Design and Construct of CAR and Membrane-Anchored Cytokines

This example illustrates the design and constructs of the CAR and membrane-anchored cytokines according to some embodiments of the present disclosure.

Each of the constructs mentioned below comprising a targeting moiety uses the same CAR19, the sequence information of CAR19 is listed in Table 5A-5B. Different constructs are used to obtain a final expression of different cytokines and CAR19 combination on cell surface, which are summarized in Table A-1 below.

TABLE A-1
EXPRESSION OF CYTOKINE(S)
AND CAR(S) ON CELLS SURFACE

| Categories | Final expression on cell surface |
|---|---|
| CAR and single cytokine | CAR19, IL-2 |
| | CAR19, IL-4 |
| | CAR19, IL-7 |
| | CAR19, IL-9 |
| | CAR19, IL-10 |
| | CAR19, IL-12p40 |
| | CAR19, IL-15 |
| | CAR19, IL-18 |
| | CAR19, IL-21 |
| | CAR19, IL- 36γ |
| CAR and two cytokines | CAR19, IL-12p40, IL-7 |
| | CAR19, IL-12p40, IL-15 |
| | CAR19, IL-12p40, IL-21 |
| | CAR19, IL-15, IL-2 |
| | CAR19, IL-15, IL-7 |
| | CAR19, IL-15, IL-12p40 |
| | CAR19, IL-15, IL-21 |
| | CAR19, IL-21, IL-7 |
| | CAR19, IL-21, IL-12p40 |
| | CAR19, IL-21, IL-15 |
| | CAR19, IL-4, IL-15 |
| CAR and three cytokines | CAR19, IL-2, IL-7, IL-15 |
| | CAR19, IL-12p40, IL-7, IL-21 |
| | CAR19, IL-12p40, IL-15, IL-21 |
| | CAR19, IL-15, IL-7, IL-21 |
| | CAR19, IL-15, IL-12p40, IL-21 |
| | CAR19, IL-15, IL-21, IL-12p40 |
| | CAR19, IL-21, IL-7, IL-15 |
| | CAR19, IL-21, IL-12p40, IL-15 |

Viral Vector Constructs used in the experiment comprise a single cytokine and anchor, two cytokines with anchors, three cytokines with anchors, each of them combined with CAR, such as CAR19. The constructs are listed in Table A-2 to A-7 according to their types.

TABLE A-2
VIRAL VECTOR CONSTRUCTS WITH ONE CYTOKINE

| General structure: | | |
|---|---|---|
| SP-Cytokine- | | |
| Anchoring | | Amino Acid |
| structure | Nucleic Acid sequence | sequence |
| SP-IL-2-Anchoring | gctgtgatggcccctagaaccctgctgctgctgctgagcggcgccctggccct | AVMAPRTLLLLLSGAL |
| structure | gacacagacctgggccgcccctaccagcagctccaccaagaagacccagct | ALTQTWA |
| | gcagctggagcacctcctgctggacctgcagatgatcctgaacggcatcaac | APTSSSTKKTQLQLEH |
| | aactataagaatcctaagctgacaagaatgctgacctttaagttctacatgcc | LLLDLQMILNGINNYK |
| | taagaaagctaccgagctgaagcacctgcaatgtctggaagaggagctgaa | NPKLTRMLTFKFYMP |
| | acctctggaagaggtgctgaatctggcccagagcaaaaacttccacctcaga | KKATELKHLQCLEEEL |
| | cctagagatctgatcagcaacatcaacgtgatcgtgctggaactgaagggca | KPLEEVLNLAQSKNF |
| | gcgagacaacattcatgtgcgagtacgccgacgagacagccaccattgtgg | HLRPRDLISNINVIVLE |
| | aattcctgaaccggtggatcaccttctgccagtccatcatctctacactgaccg | LKGSETTFMCEYADE |
| | aattccttgaaaatggtgggacatccttatcagagaaaacagttcttctgctg | TATIVEFLNRWITFCQ |
| | gtgactccatttctggcagcagcctggagccttcatccc (SEQ ID NO: | SIISTLTEFLENGGTSLS |
| | 611) | EKTVLLLVTPFLAAAW |
| | | SLHP |
| | | (SEQ ID NO: 618) |
| SP-IL-7-Anchoring | gctgtgatggcccctagaaccctgctgctgctgctgagcggcgccctggccct | AVMAPRTLLLLLSGAL |
| structure | gacacagacctgggccgattgtgatattgaaggtaaagatggcaaacaata | ALTQTWADCDIEGKD |
| | tgagagtgttctaatggtcagcatcgatcaattattggacagcatgaaagaa | GKQYESVLMVSIDQL |
| | attggtagcaattgcctgaataatgaatttaacttttttaaaagacatatctgt | LDSMKEIGSNCLNNE |
| | gatgctaataaggaaggtatgtttttattccgtgctgctcgcaagttgaggca | FNFFKRHICDANKEG |
| | atttcttaaaatgaatagcactggtgattttgatctccacttattaaaagtttc | MFLFRAARKLRQFLK |
| | agaaggcacaacaatactgttgaactgcactggccaggttaaaggaagaaaa | MNSTGDFDLHLLKVS |
| | ccagctgccctgggtgaagcccaaccaacaaagagtttggaagaaaataaa | EGTTILLNCTGQVKGR |
| | tctttaaaggaacagaaaaaactgaatgacttgtgtttcctaaagagactatt | KPAALGEAQPTKSLEE |
| | acaagagataaaaacttgttggaataaaattttgatgggcactaaagaaca | NKSLKEQKKLNDLCFL |
| | cgaattccttgaaaatggtgggacatccttatcagagaaaacagttcttctgc | KRLLQEIKTCWNKIL |
| | tggtgactccatttctggcagcagcctggagccttcatccc (SEQ ID | MGTKEHEFLENGGTS |
| | NO: 612) | LSEKTVLLLVTPFLAAA |
| | | WSLHP (SEQ ID |
| | | NO: 619) |
| SP-IL-9-Anchoring | ctgctggccatggtgctgacaagcgccctgctgctgtgctctgtggccgggca | LLAMVLTSALLLCSVA |
| structure | ggggtgccctaccctggctggaatcctggacatcaacttcctgatcaacaag | GQGCPTLAGILDINFLI |
| | atgcaggaggaccccgcttccaagtgccactgctccgccaatgtgacctcctg | NKMQEDPASKCHCS |
| | cctgtgtctgggcattccctccgacaactgtactagaccctgcttcagcgagc | ANVTSCLCLGIPSDNC |
| | gcctgagccagatgactaacaccaccatgcagacccgctaccccctgatctt | TRPCFSERLSQMTNT |
| | ctcccgcgtcaagaagtccgtcgaagtgctgaagaacaacaagtgcccctac | TMQTRYPLIFSRVKKS |
| | ttctcctgcgagcagccctgcaaccagaccaccgccggcaacgccctgacct | VEVLKNNKCPYFSCE |
| | tcctgaaaagcctgctggagatcttccagaaggagaaaatgcgcggcatga | QPCNQTTAGNALTFL |
| | gaggcaagatcgagtttctggagaacggagggacaagcctgagcgagaag | KSLLEIFQKEKMRGM |
| | acagtgctgctgctggtgaccccatttctggccgcagcatggagcctgcaccc | RGKIEFLENGGTSLSE |
| | c (SEQ ID NO: 613) | KTVLLLVTPFLAAAWS |
| | | LHP (SEQ ID NO: |
| | | 620) |
| SP-IL-12p40- | gctgtgatggcccctagaaccctgctgctgctgctgagcggcgccctggccct | AVMAPRTLLLLLSGAL |
| Anchoring | gacacagacctgggcccaccatcatcatcatcatgaattggactacaaagac | ALTQTWAIWELKKDV |
| structure | gatgacgacaaggaattgatatgggaactgaagaaagatgtttatgtcgtag | YVVELDWYPDAPGE |
| | aattggattggtatccggatgcccctggagaaatggtggtcctcacctgtgac | MVVLTCDTPEEDGIT |
| | acccctgaagaagatggtatcacctggaccttggaccagagcagtgaggtct | WTLDQSSEVLGSGKT |
| | taggctctggcaaaaccctgaccatccaagtcaaagagtttggagatgctgg | LTIQVKEFGDAGQYT |
| | ccagtacacctgtcacaaaggaggcgaggttctaagccattcgctcctgctgc | CHKGGEVLSHSLLLLH |
| | ttcacaaaaaggaagatggaatttggtccactgatattttaaaggaccagaa | KKEDGIWSTDILKDQ |
| | agaacccaaaaataagacctttctaagatgcgaggccaagaattattctgga | KEPKNKTFLRCEAKNY |
| | cgtttcacctgctggtggctgacgacaatcagtactgatttgacattcagtgtc | SGRFTCWWLTTISTD |
| | aaaagcagcagaggctcttctgacccccaaggggtgacgtgcggagctgct | LTFSVKSSRGSSDPQG |
| | acactctctgcagagagagtcagaggggacaacaaggagtatgagtactca | VTCGAATLSAERVRG |
| | gtggagtgccaggaggacagtgcctgcccagctgctgaggagagtctgccc | DNKEYEYSVECQEDS |
| | attgaggtcatggtggatgccgttcacaagctcaagtatgaaaactacacca | ACPAAEESLPIEVMV |
| | gcagcttcttcatcagggacatcatcaaacctgacccacccaagaacttgca | DAVHKLKYENYTSSFF |
| | gctgaagccattaaagaattctcggcaggtggaggtcagctgggagtaccct | IRDIIKPDPPKNLQLKP |
| | gacacctggagtactccacattcctacttctccctgacattctgcgttcaggtc | LKNSRQVEVSWEYPD |
| | cagggcaagagcaagagagaaaagaaagatagagtcttcacggacaaga | TWSTPHSYFSLTFCV |
| | cctcagccacggtcatctgccgcaaaaatgccagcattagcgtgcgggccca | QVQGKSKREKKDRVF |
| | ggaccgctactatagctcatcttggagcgaatgggcatctgtgccctgcagtg | TDKTSATVICRKNASI |
| | aattccttgaaaatggtgggacatccttatcagagaaaacagttcttctgctg | SVRAQDRYYSSSWSE |
| | gtgactccatttctggcagcagcctggagccttcatccc (SEQ ID NO: | WASVPCSEFLENGGT |
| | 614) | SLSEKTVLLLVTPFLAA |
| | | AWSLHP (SEQ ID |
| | | NO: 621) |
| SP-IL-15-Anchoring | ggcaccagcctgctgtgctggatggcactgtgcctgctgggagcagaccacg | GTSLLCWMALCLLGA |
| structure | ccgatgccggcatccacgtgttcatcctgggctgcttcagcgccggacttcct | DHADAGIHVFILGCFS |
| | aaaacagaggccaactgggtgaacgtgattagcgacctgaagaagatcga | AGLPKTEANWVNVIS |
| | ggacctgatccagagcatgcacatcgacgccaccctgtacaccgagagcga | DLKKIEDLIQSMHIDA |
| | tgtgcatcctagctgcaaggtgaccgccatgaagtgcttcctgctggagctgc | TLYTESDVHPSCKVTA |
| | aggtgatcagcctggagagcggagatgccagcattcacgacacagtggaaa | MKCFLLELQVISLESG |
| | atctgatcatcctggccaacaacagcctgagcagcaacggcaatgtgaccg | DASIHDTVENLIILAN |
| | agagcggctgtaaggagtgcgaggaactggaggagaagaacatcaaggag | NSLSSNGNVTESGCK |
| | ttcctgcagagcttcgtgcacatcgtgcagatgttcatcaacaccagcagagc | ECEELEEKNIKEFLQSF |
| | cgaattccttgaaaatggtgggacatccttatcagagaaaacagttcttctgc | VHIVQMFINTSRAEFL |
| | tggtgactccatttctggcagcagcctggagccttcatccc (SEQ ID | ENGGTSLSEKTVLLLV |
| | NO: 615) | TPFLAAAWSLHP |
| | | (SEQ ID NO: 622) |
| SP-IL-18-Anchoring | Atggccgctgagcccgtggaggacaactgcatcaacttcgtggccatgaagt | MAAEPVEDNCINFVA |
| structure | tcatcgacaacaccctgtactttatcgccgaggacgacgagaacctggagag | MKFIDNTLYFIAEDDE |
| | cgactactttggcaagctggagagcaagctgagcgtgatccggaacctgaa | NLESDYFGKLESKLSVI |
| | cgaccaggtgctgttcatcgaccagggcaatcggcctctgtttgaggacatga | RNLNDQVLFIDQGNR |
| | ccgacagcgactgcagagacaacgcacccagaaccatcttcatcatctccat | PLFEDMTDSDCRDN |
| | gtacaaggactcccagccaaggggcatggccgtgaccatcagcgtgaaatg | APRTIFIISMYKDSQP |
| | cgagaaaatcagcacactgtcatgcgagaacaagatcatcagcttcaagga | RGMAVTISVKCEKIST |
| | aatgaacccccccgacaacatcaaggacacaaaaagcgacatcatcttcttc | LSCENKIISFKEMNPP |
| | cagagatccgtccccggccacgacaacaaaatgcagttcgagagctcctcct | DNIKDTKSDIIFFQRSV |
| | acgagggctacttcctggcctgcgaaaaagaaagagacctgttcaagctgat | PGHDNKMQFESSSYE |
| | cctgaagaaggaggacgagctgggcgacagaagcatcatgttcaccgtgca | GYFLACEKERDLFKLIL |
| | gaacgaggacgagttcctggagaacggggggacaagcctgtccgagaaga | KKEDELGDRSIMFTV |
| | ccgtgctgctgctggtcactcccttcctggccgccgcctggagcctgcatccc | QNEDEFLENGGTSLS |
| | (SEQ ID NO: 606) | EKTVLLLVTPFLAAAW |
| | | SLHP (SEQ ID NO: |
| | | 607) |
| SP-IL-21-Anchoring | ggcaccagcctgctgtgctggatggcactgtgcctgctgggagcagaccacg | GTSLLCWMALCLLGA |
| structure | ccgatgcccagggccaggacagacacatgatcagaatgagacagctgatcg | DHADAQGQDRHMIR |
| | acatcgtggaccagctgaagaactacgtgaacgacctggtgcccgagttcct | MRQLIDIVDQLKNYV |
| | gcccgctcctgaagacgtggaaacaaactgtgagtggagcgctttcagctgc | NDLVPEFLPAPEDVET |
| | ttccagaaggcccagctgaagagcgccaataccggaaacaatgagagaatc | NCEWSAFSCFQKAQL |
| | atcaacgtgagcatcaagaagctgaagagaaagccccccagcaccaacgc | KSANTGNNERIINVSI |
| | cggaagaagacaaaaacatagactgacctgccccagctgcgatagctacga | KKLKRKPPSTNAGRR |
| | gaaaaagccccccaaggagttcctggagagattcaagagcctgctgcagaa | QKHRLTCPSCDSYEKK |
| | gatgatccaccagcacctgagcagcagaacccacggctctgaagatagcag | PPKEFLERFKSLLQKM |
| | agctgaattccttgaaaatggtgggacatccttatcagagaaaacagttcttc | IHQHLSSRTHGSEDSR |
| | tgctggtgactccatttctggcagcagcctggagccttcatccc (SEQ ID | AEFLENGGTSLSEKTV |
| | NO: 616) | LLLVTPFLAAAWSLHP |
| | | (SEQ ID NO: 623) |
| SP-IL-36γ- | atgagagggacccccggggacgccgacggaggaggaagagcagtgtacca | MRGTPGDADGGGR |
| Anchoring | gagcatgtgcaagcccatcaccgggaccatcaacgacctgaaccagcaggt | AVYQSMCKPITGTIN |
| structure | gtggaccctgcagggacagaacctggtggccgtgcccagaagcgatagcgt | DLNQQVWTLQGQN |
| | gacacccgtgaccgtcgccgtcattacctgcaagtaccccgaagccctggaa | LVAVPRSDSVTPVTV |
| | cagggcagaggcgatcccatctacctggggattcaaaaccccgaaatgtgcc | AVITCKYPEALEQGRG |
| | tgtactgcgagaaggtcggcgagcagcctaccctgcagctgaaggaacaga | DPIYLGIQNPEMCLYC |
| | aaatcatggacctgtacggacagcctgagcccgtgaaacccttcctgttctac | EKVGEQPTLQLKEQKI |
| | agagctaagaccggccgcaccagcacactggagagcgtcgccttccccgac | MDLYGQPEPVKPFLF |
| | tggttcatcgccagcagcaaaagagaccagcccatcatcctgacctcagagc | YRAKTGRTSTLESVAF |
| | tgggaaagagctacaacaccgccttcgagctgaacatcaacgatgagttcct | PDWFIASSKRDQPIIL |
| | ggagaacggggggacaagcctgtccgagaagaccgtgctgctgctggtcac | TSELGKSYNTAFELNI |
| | tcccttcctggccgccgcctggagcctgcatccc (SEQ ID NO: 617) | NDEFLENGGTSLSEKT |
| | | VLLLVTPFLAAAWSLH |
| | | P (SEQ ID NO: 624) |

TABLE A-3
VIRAL VECTOR CONSTRUCTS WITH TWO CYTOKINES
General structure: SP1-Cytokine1-Anchoring structure 1-Linker-SP2-Cytokine2-Anchoring
structure 2

| | | Amino Acid |
|---|---|---|
| Construct | Nucleic Acid Sequence | Sequence |
| SP1-IL-12p40- | gctgtgatggcccctagaaccctgctgctgctgctgagcggcgccctggccctgac | AVMAPRTLLLLLSGAL |
| Anchoring | acagacctgggccatatgggaactgaagaaagatgtttatgtcgtagaattggatt | ALTQTWAIWELKKDV |
| structure 1- | ggtatccggatgcccctggagaaatggtggtcctcacctgtgacacccctgaagaa | YVVELDWYPDAPGE |
| T2A-SP2-IL-21- | gatggtatcacctggaccttggaccagagcagtgaggtcttaggctctggcaaaac | MVVLTCDTPEEDGIT |
| Anchoring | cctgaccatccaagtcaaagagtttggagatgctggccagtacacctgtcacaaag | WTLDQSSEVLGSGKT |
| structure 2 | gaggcgaggttctaagccattcgctcctgctgcttcacaaaaaggaagatggaatt | LTIQVKEFGDAGQYT |
| | tggtccactgatattttaaaggaccagaaagaacccaaaaataagacctttctaag | CHKGGEVLSHSLLLLH |
| | atgcgaggccaagaattattctggacgtttcacctgctggtggctgacgacaatca | KKEDGIWSTDILKDQ |
| | gtactgatttgacattcagtgtcaaaagcagcagaggctcttctgacccccaaggg | KEPKNKTFLRCEAKNY |
| | gtgacgtgcggagctgctacactctctgcagagagagtcagaggggacaacaagg | SGRFTCWWLTTISTD |
| | agtatgagtactcagtggagtgccaggaggacagtgcctgcccagctgctgagga | LTFSVKSSRGSSDPQG |
| | gagtctgcccattgaggtcatggtggatgccgttcacaagctcaagtatgaaaact | VTCGAATLSAERVRG |
| | acaccagcagcttcttcatcagggacatcatcaaacctgacccacccaagaacttg | DNKEYEYSVECQEDS |
| | cagctgaagccattaaagaattctcggcaggtggaggtcagctgggagtaccctg | ACPAAEESLPIEVMV |
| | acacctggagtactccacattcctacttctccctgacattctgcgttcaggtccagg | DAVHKLKYENYTSSFF |
| | gcaagagcaagagagaaaagaaagatagagtcttcacggacaagacctcagccac | IRDIIKPDPPKNLQLKP |
| | ggtcatctgccgcaaaaatgccagcattagcgtgcgggcccaggaccgctactata | LKNSRQVEVSWEYPD |
| | gctcatcttggagcgaatgggcatctgtgccctgcagtagagccgagttcctggag | TWSTPHSYFSLTFCV |
| | aacggaggaacaagcctgagcgagaaaacagtgctgctgctggtgaccccattcc | QVQGKSKREKKDRVF |
| | tggcagccgcctggagcctgcaccccggctccggcgagggcaggggaagtctact | TDKTSATVICRKNASI |
| | aacatgcggggacgtggaggaaaatcccggccccggaacaagcctgctgtgctgg | SVRAQDRYYSSSWSE |
| | atggctctgtgcctgctgggcgctgaccacgccgacgctcagggccaggacagac | WASVPCSRAEFLENG |
| | acatgatcagaatgagacagctgatcgacatcgtggaccagctgaagaactacgt | GTSLSEKTVLLLVTPFL |
| | gaacgacctggtgcccgagttcctgcccgctcctgaagacgtggaaacaaactgtg | AAAWSLHPGSGEGR |
| | agtggagcgctttcagctgcttccagaaggcccagctgaagagcgccaataccgg | GSLLTCGDVEENPGP |
| | aaacaatgagagaatcatcaacgtgagcatcaagaagctgaagagaaagccccc | GTSLLCWMALCLLGA |
| | cagcaccaacgccggaagaagacaaaaacatagactgacctgccccagctgcga | DHADAQGQDRHMIR |
| | tagctacgagaaaaagccccccaaggagttcctggagagattcaagagcctgctg | MRQLIDIVDQLKNYV |
| | cagaagatgatccaccagcacctgagcagcagaacccacggctctgaagatagc | NDLVPEFLPAPEDVET |
| | agagctagagccgaattcctggagaacggagggacaagcctgagcgagaagaca | NCEWSAFSCFQKAQL |
| | gtgctgctgctggtgaccccatttctggccgcagcatggagcctgcacccc (SEQ | KSANTGNNERIINVSI |
| | ID NO: 600) | KKLKRKPPSTNAGRR |
| | | QKHRLTCPSCDSYEKK |
| | | PPKEFLERFKSLLQKM |
| | | IHQHLSSRTHGSEDSR |
| | | ARAEFLENGGTSLSEK |
| | | TVLLLVTPFLAAAWSL |
| | | HP (SEQ ID NO: |
| | | 603) |
| SP1-IL-15- | ggcaccagcctgctgtgctggatggcactgtgcctgctgggagcagaccacgccga | GTSLLCWMALCLLGA |
| Anchoring | tgccggcatccacgtgttcatcctgggctgcttcagcgccggacttcctaaaacaga | DHADAGIHVFILGCFS |
| structure 1- | ggccaactgggtgaacgtgattagcgacctgaagaagatcgaggacctgatccag | AGLPKTEANWVNVIS |
| P2A-SP2-IL- | agcatgcacatcgacgccaccctgtacaccgagagcgatgtgcatcctagctgcaa | DLKKIEDLIQSMHIDA |
| 12p40- | ggtgaccgccatgaagtgcttcctgctggagctgcaggtgatcagcctggagagcg | TLYTESDVHPSCKVTA |
| Anchoring | gagatgccagcattcacgacacagtggaaaatctgatcatcctggccaacaacag | MKCFLLELQVISLESG |
| structure 2 | cctgagcagcaacggcaatgtgaccgagagcggctgtaaggagtgcgaggaact | DASIHDTVENLIILAN |
| | ggaggagaagaacatcaaggagttcctgcagagcttcgtgcacatcgtgcagatg | NSLSSNGNVTESGCK |
| | ttcatcaacaccagcagagccagggccgagttccttgaaaatggtgggacatcctt | ECEELEEKNIKEFLQSF |
| | atcagagaaaacagttcttctgctggtgactccatttctggcagcagcctggagcct | VHIVQMFINTSRARA |
| | tcatcccggctccggcgccacgaacttctctctgttaaagcaagcaggagacgtgg | EFLENGGTSLSEKTVL |
| | aagaaaaccccggtcccgctgtgatggcccctagaaccctgctgctgctgctgagc | LLVTPFLAAAWSLHP |
| | ggcgccctggccctgacacagacctgggccatatgggaactgaagaaagatgttt | GSGATNFSLLKQAGD |
| | atgtcgtagaattggattggtatccggatgcccctggagaaatggtggtcctcacct | VEENPGPAVMAPRTL |
| | gtgacacccctgaagaagatggtatcacctggaccttggaccagagcagtgaggt | LLLLSGALALTQTWAI |
| | cttaggctctggcaaaaccctgaccatccaagtcaaagagtttggagatgctggcc | WELKKDVYVVELDW |
| | agtacacctgtcacaaaggaggcgaggttctaagccattcgctcctgctgcttcaca | YPDAPGEMVVLTCDT |
| | aaaaggaagatggaatttggtccactgatattttaaaggaccagaaagaacccaa | PEEDGITWTLDQSSE |
| | aaataagacctttctaagatgcgaggccaagaattattctggacgtttcacctgctg | VLGSGKTLTIQVKEFG |
| | gtggctgacgacaatcagtactgatttgacattcagtgtcaaaagcagcagaggct | DAGQYTCHKGGEVLS |
| | cttctgacccccaaggggtgacgtgcggagctgctacactctctgcagagagagtc | HSLLLLHKKEDGIWST |
| | agaggggacaacaaggagtatgagtactcagtggagtgccaggaggacagtgcc | DILKDQKEPKNKTFLR |
| | tgcccagctgctgaggagagtctgcccattgaggtcatggtggatgccgttcacaa | CEAKNYSGRFTCWW |
| | gctcaagtatgaaaactacaccagcagcttcttcatcagggacatcatcaaacctg | LTTISTDLTFSVKSSRG |
| | acccacccaagaacttgcagctgaagccattaaagaattctcggcaggtggaggt | SSDPQGVTCGAATLS |
| | cagctgggagtaccctgacacctggagtactccacattcctacttctccctgacatt | AERVRGDNKEYEYSV |
| | ctgcgttcaggtccagggcaagagcaagagagaaaagaaagatagagtcttcacg | ECQEDSACPAAEESLP |
| | gacaagacctcagccacggtcatctgccgcaaaaatgccagcattagcgtgcggg | IEVMVDAVHKLKYEN |
| | cccaggaccgctactatagctcatcttggagcgaatgggcatctgtgccctgcagta | YTSSFFIRDIIKPDPPK |
| | gagccgagttcctggagaacggaggaacaagcctgagcgagaaaacagtgctgc | NLQLKPLKNSRQVEV |
| | tgctggtgaccccattcctggcagccgcctggagcctgcacccc (SEQ ID | SWEYPDTWSTPHSYF |
| | NO: 601) | SLTFCVQVQGKSKRE |
| | | KKDRVFTDKTSATVIC |
| | | RKNASISVRAQDRYYS |
| | | SSWSEWASVPCSRAE |
| | | FLENGGTSLSEKTVLLL |
| | | VTPFLAAAWSLHP |
| | | (SEQ ID NO: 604) |
| SP1-IL-15- | ggcaccagcctgctgtgctggatggcactgtgcctgctgggagcagaccacgccga | GTSLLCWMALCLLGA |
| Anchoring | tgccggcatccacgtgttcatcctgggctgcttcagcgccggacttcctaaaacaga | DHADAGIHVFILGCFS |
| structure 1- | ggccaactgggtgaacgtgattagcgacctgaagaagatcgaggacctgatccag | AGLPKTEANWVNVIS |
| P2A-SP2-IL-21- | agcatgcacatcgacgccaccctgtacaccgagagcgatgtgcatcctagctgcaa | DLKKIEDLIQSMHIDA |
| Anchoring | ggtgaccgccatgaagtgcttcctgctggagctgcaggtgatcagcctggagagcg | TLYTESDVHPSCKVTA |
| structure 2 | gagatgccagcattcacgacacagtggaaaatctgatcatcctggccaacaacag | MKCFLLELQVISLESG |
| | cctgagcagcaacggcaatgtgaccgagagcggctgtaaggagtgcgaggaact | DASIHDTVENLIILAN |
| | ggaggagaagaacatcaaggagttcctgcagagcttcgtgcacatcgtgcagatg | NSLSSNGNVTESGCK |
| | ttcatcaacaccagcagagccagggccgagttccttgaaaatggtgggacatcctt | ECEELEEKNIKEFLQSF |
| | atcagagaaaacagttcttctgctggtgactccatttctggcagcagcctggagcct | VHIVQMFINTSRARA |
| | tcatcccggctccggcgccacgaacttctctctgttaaagcaagcaggagacgtgg | EFLENGGTSLSEKTVL |
| | aagaaaaccccggtcccggaacaagcctgctgtgctggatggctctgtgcctgctg | LLVTPFLAAAWSLHP |
| | ggcgctgaccacgccgacgctcagggccaggacagacacatgatcagaatgaga | GSGATNFSLLKQAGD |
| | cagctgatcgacatcgtggaccagctgaagaactacgtgaacgacctggtgcccg | VEENPGPGTSLLCW |
| | agttcctgcccgctcctgaagacgtggaaacaaactgtgagtggagcgctttcagc | MALCLLGADHADAQ |
| | tgcttccagaaggcccagctgaagagcgccaataccggaaacaatgagagaatc | GQDRHMIRMRQLIDI |
| | atcaacgtgagcatcaagaagctgaagagaaagccccccagcaccaacgccgga | VDQLKNYVNDLVPEF |
| | agaagacaaaaacatagactgacctgccccagctgcgatagctacgagaaaaag | LPAPEDVETNCEWSA |
| | ccccccaaggagttcctggagagattcaagagcctgctgcagaagatgatccacc | FSCFQKAQLKSANTG |
| | agcacctgagcagcagaacccacggctctgaagatagcagagctagagccgaatt | NNERIINVSIKKLKRKP |
| | cctggagaacggagggacaagcctgagcgagaagacagtgctgctgctggtgac | PSTNAGRRQKHRLTC |
| | cccatttctggccgcagcatggagcctgcacccc (SEQ ID NO: 602) | PSCDSYEKKPPKEFLE |
| | | RFKSLLQKMIHQHLSS |
| | | RTHGSEDSRARAEFLE |
| | | NGGTSLSEKTVLLLVT |
| | | PFLAAAWSLHP |
| | | (SEQ ID NO: 605) |
| SP1-IL-7- | gctgtgatggcccctagaaccctgctgctgctgctgagcggcgccctggccctgac | |
| Anchoring | acagacctgggccgattgtgatattgaaggtaaagatggcaaacaatatgagagt | |
| structure 1- | gttctaatggtcagcatcgatcaattattggacagcatgaaagaaattggtagcaa | |
| IRES-SP2-IL-15- | ttgcctgaataatgaatttaacttttttaaaagacatatctgtgatgctaataagga | |
| Anchoring | aggtatgtttttattccgtgctgctcgcaagttgaggcaatttcttaaaatgaatag | |
| structure 2 | cactggtgattttgatctccacttattaaaagtttcagaaggcacaacaatactgtt | |
| | gaactgcactggccaggttaaaggaagaaaaccagctgccctgggtgaagcccaacc | |
| | aacaaagagtttggaagaaaataaatctttaaaggaacagaaaaaactgaatga | |
| | cttgtgtttcctaaagagactattacaagagataaaaacttgttggaataaaatttt | |
| | gatgggcactaaagaacacgaattccttgaaaatggtgggacatccttatcagag | |
| | aaaacagttcttctgctggtgactccatttctggcagcagcctggagccttcatccc | |
| | taacccctctccctcccccccccctaacgttactggccgaagccgcttggaataagg | |
| | ccggtgtgcgtttgtctatatgttattttccaccatattgccgtcttttggcaatgt | |
| | gagggcccggaaacctggccctgtcttcttgacgagcattcctaggggtctttcccc | |
| | tctcgccaaaggaatgcaaggtctgttgaatgtcgtgaaggaagcagttcctctgga | |
| | agcttcttgaagacaaacaacgtctgtagcgaccctttgcaggcagcggaacccccc | |
| | acctggcgacaggtgcctctgcggccaaaagccacgtgtataagatacacctgcaa | |
| | aggcggcacaaccccagtgccacgttgtgagttggatagttgtggaaagagtcaa | |
| | atggctctcctcaagcgtattcaacaaggggctgaaggatgcccagaaggtacccc | |
| | attgtatgggatctgatctggggcctcggtgcacatgctttacatgtgtttagtcga | |
| | ggttaaaaaaacgtctaggccccccgaaccacggggacgtggttttcctttgaaaaa | |
| | cacgatgataagccaccatgggcaccagcctgctgtgctggatggcactgtgcctg | |
| | ctgggagcagaccacgccgatgccggcatccacgtgttcatcctgggctgcttcag | |
| | cgccggacttcctaaaacagaggccaactgggtgaacgtgattagcgacctgaag | |
| | aagatcgaggacctgatccagagcatgcacatcgacgccaccctgtacaccgaga | |
| | gcgatgtgcatcctagctgcaaggtgaccgccatgaagtgcttcctgctggagctg | |
| | caggtgatcagcctggagagcggagatgccagcattcacgacacagtggaaaatc | |
| | tgatcatcctggccaacaacagcctgagcagcaacggcaatgtgaccgagagcgg | |
| | ctgtaaggagtgcgaggaactggaggagaagaacatcaaggagttcctgcagag | |
| | cttcgtgcacatcgtgcagatgttcatcaacaccagcagagccgagttcctggaga | |
| | acggtggcaccagcctttccgagaagaccgtcttgctgctggtgactccttttctg | |
| | gccgcggcttggtctctccacccc (SEQ ID NO: 625) | |
| SP1-IL-21- | ggaacaagcctgctgtgctggatggctctgtgcctgctgg | GTSLLCWMALCLLGA |
| Anchoring | gcgctgaccacgccgacgctcagggccaggacagacacat | DHADAQGQDRHMIR |
| structure 1- | gatcagaatgagacagctgatcgacatcgtggaccagctg | MRQLIDIVDQLKNYV |
| T2A-SP2-IL- | aagaactacgtgaacgacctggtgcccgagttcctgcccg | NDLVPEFLPAPEDVET |
| 12p40- | ctcctgaagacgtggaaacaaactgtgagtggagcgcttt | NCEWSAFSCFQKAQL |
| Anchoring | cagctgcttccagaaggcccagctgaagagcgccaatacc | KSANTGNNERIINVSI |
| structure 2 | ggaaacaatgagagaatcatcaacgtgagcatcaagaagc | KKLKRKPPSTNAGRR |
| | tgaagagaaagccccccagcaccaacgccggaagaagaca | QKHRLTCPSCDSYEKK |
| | aaaacatagactgacctgccccagctgcgatagctacgag | PPKEFLERFKSLLQKM |
| | aaaaagccccccaaggagttcctggagagattcaagagcc | IHQHLSSRTHGSEDSR |
| | tgctgcagaagatgatccaccagcacctgagcagcagaac | ARAEFLENGGTSLSEK |
| | ccacggctctgaagatagcagagctagagccgaattcctg | TVLLLVTPFLAAAWSL |
| | gagaacggagggacaagcctgagcgagaagacagtgctgc | HPAGTRGSGEGRGSL |
| | tgctggtgaccccatttctggccgcagcatggagcctgca | LTCGDVEENPGPLEA |
| | ccccgctggaacgcgtggctccggcgagggcaggggaagt | VMAPRTLLLLLSGALA |
| | cttctaacatgcggggacgtggaggaaaatcccggcccac | LTQTWAIWELKKDVY |
| | tcgaggctgtgatggcccctagaaccctgctgctgctgct | VVELDWYPDAPGEM |
| | gagcggcgccctggccctgacacagacctgggccatatgg | VVLTCDTPEEDGITW |
| | gaactgaagaaagatgtttatgtcgtagaattggattggt | TLDQSSEVLGSGKTLT |
| | atccggatgcccctggagaaatggtggtcctcacctgtga | IQVKEFGDAGQYTCH |
| | cacccctgaagaagatggtatcacctggaccttggaccag | KGGEVLSHSLLLLHKK |
| | agcagtgaggtcttaggctctggcaaaaccctgaccatcc | EDGIWSTDILKDQKE |
| | aagtcaaagagtttggagatgctggccagtacacctgtca | PKNKTFLRCEAKNYS |
| | caaaggaggcgaggttctaagccattcgctcctgctgctt | GRFTCWWLTTISTDL |
| | cacaaaaaggaagatggaatttggtccactgatattttaa | TFSVKSSRGSSDPQG |
| | aggaccagaaagaacccaaaaataagacctttctaagatg | VTCGAATLSAERVRG |
| | cgaggccaagaattattctggacgtttcacctgctggtgg | DNKEYEYSVECQEDS |
| | ctgacgacaatcagtactgatttgacattcagtgtcaaaa | ACPAAEESLPIEVMV |
| | gcagcagaggctcttctgacccccaaggggtgacgtgcgg | DAVHKLKYENYTSSFF |
| | agctgctacactctctgcagagagagtcagaggggacaac | IRDIIKPDPPKNLQLKP |
| | aaggagtatgagtactcagtggagtgccaggaggacagtg | LKNSRQVEVSWEYPD |
| | cctgcccagctgctgaggagagtctgcccattgaggtcat | TWSTPHSYFSLTFCV |
| | ggtggatgccgttcacaagctcaagtatgaaaactacacc | QVQGKSKREKKDRVF |
| | agcagcttcttcatcagggacatcatcaaacctgacccac | TDKTSATVICRKNASI |
| | ccaagaacttgcagctgaagccattaaagaattctcggca | SVRAQDRYYSSSWSE |
| | ggtggaggtcagctgggagtaccctgacacctggagtact | WASVPCSRAEFLENG |
| | ccacattcctacttctccctgacattctgcgttcaggtcc | GTSLSEKTVLLLVTPFL |
| | agggcaagagcaagagagaaaagaaagatagagtcttcac | AAAWSLHP (SEQ ID |
| | ggacaagacctcagccacggtcatctgccgcaaaaatgcc | NO: 639) |
| | agcattagcgtgcgggcccaggaccgctactatagctcat | |
| | cttggagcgaatgggcatctgtgccctgcagtagagccga | |
| | gttcctggagaacggaggaacaagcctgagcgagaaaaca | |
| | gtgctgctgctggtgaccccattcctggcagccgcctgga | |
| | gcctgcacccc (SEQ ID NO: 626) | |
| SP1-IL-21- | ggaacaagcctgctgtgctggatggctctgtgcctgctgggcgctgaccacgccga | GTSLLCWMALCLLGA |
| Anchoring | cgctcagggccaggacagacacatgatcagaatgagacagctgatcgacatcgtg | DHADAQGQDRHMIR |
| structure 1- | gaccagctgaagaactacgtgaacgacctggtgcccgagttcctgcccgctcctga | MRQLIDIVDQLKNYV |
| T2A-SP2-IL-15- | agacgtggaaacaaactgtgagtggagcgctttcagctgcttccagaaggcccag | NDLVPEFLPAPEDVET |
| Anchoring | ctgaagagcgccaataccggaaacaatgagagaatcatcaacgtgagcatcaag | NCEWSAFSCFQKAQL |
| structure 2 | aagctgaagagaaagccccccagcaccaacgccggaagaagacaaaaacatag | KSANTGNNERIINVSI |
| | actgacctgccccagctgcgatagctacgagaaaaagccccccaaggagttcctg | KKLKRKPPSTNAGRR |
| | gagagattcaagagcctgctgcagaagatgatccaccagcacctgagcagcaga | QKHRLTCPSCDSYEKK |
| | acccacggctctgaagatagcagagctagagccgaattcctggagaacggaggg | PPKEFLERFKSLLQKM |
| | acaagcctgagcgagaagacagtgctgctgctggtgaccccatttctggccgcagc | IHQHLSSRTHGSEDSR |
| | atggagcctgcaccccgctggaacgcgtggctccggcgagggcaggggaagtctt | ARAEFLENGGTSLSEK |
| | ctaacatgcggggacgtggaggaaaatcccggcccactcgagggcaccagcctgc | TVLLLVTPFLAAAWSL |
| | tgtgctggatggcactgtgcctgctgggagcagaccacgccgatgccggcatccac | HPAGTRGSGEGRGSL |
| | gtgttcatcctgggctgcttcagcgccggacttcctaaaacagaggccaactgggt | LTCGDVEENPGPLEG |
| | gaacgtgattagcgacctgaagaagatcgaggacctgatccagagcatgcacatc | TSLLCWMALCLLGAD |
| | gacgccaccctgtacaccgagagcgatgtgcatcctagctgcaaggtgaccgccat | HADAGIHVFILGCFSA |
| | gaagtgcttcctgctggagctgcaggtgatcagcctggagagcggagatgccagc | GLPKTEANWVNVISD |
| | attcacgacacagtggaaaatctgatcatcctggccaacaacagcctgagcagca | LKKIEDLIQSMHIDAT |
| | acggcaatgtgaccgagagcggctgtaaggagtgcgaggaactggaggagaaga | LYTESDVHPSCKVTA |
| | acatcaaggagttcctgcagagcttcgtgcacatcgtgcagatgttcatcaacacc | MKCFLLELQVISLESG |
| | agcagagccagggccgagttccttgaaaatggtgggacatccttatcagagaaaa | DASIHDTVENLIILAN |
| | cagttcttctgctggtgactccatttctggcagcagcctggagccttcatccc | NSLSSNGNVTESGCK |
| | (SEQ ID NO: 627) | ECEELEEKNIKEFLQSF |
| | | VHIVQMFINTSRARA |
| | | EFLENGGTSLSEKTVL |
| | | LLVTPFLAAAWSLHP |
| | | (SEQ ID NO: 640) |
| SP1-IL-12p40- | GCTGTGATGGCCCCTAGAACCCTGCTGCTGCTGCTGAGCGGCG | AVMAPRTLLLLLSGAL |
| Anchoring | CCCTGGCCCTGACACAGACCTGGGCCatatgggaactgaagaaagat | ALTQTWAIWELKKDV |
| structure 1- | gtttatgtcgtagaattggattggtatccggatgcccctggagaaatggtggtcctc | YVVELDWYPDAPGE |
| T2A- | acctgtgacacccctgaagaagatggtatcacctggaccttggaccagagcagtg | MVVLTCDTPEEDGIT |
| SP2-IL-15- | aggtcttaggctctggcaaaaccctgaccatccaagtcaaagagtttggagatgct | WTLDQSSEVLGSGKT |
| Anchoring | ggccagtacacctgtcacaaaggaggcgaggttctaagccattcgctcctgctgctt | LTIQVKEFGDAGQYT |
| structure 2 | cacaaaaaggaagatggaatttggtccactgatattttaaaggaccagaaagaac | CHKGGEVLSHSLLLLH |
| | ccaaaaataagacctttctaagatgcgaggccaagaattattctggacgtttcacct | KKEDGIWSTDILKDQ |
| | gctggtggctgacgacaatcagtactgatttgacattcagtgtcaaaagcagcaga | KEPKNKTFLRCEAKNY |
| | ggctcttctgacccccaaggggtgacgtgcggagctgctacactctctgcagagag | SGRFTCWWLTTISTD |
| | agtcagaggggacaacaaggagtatgagtactcagtggagtgccaggaggacag | LTFSVKSSRGSSDPQG |
| | tgcctgcccagctgctgaggagagtctgcccattgaggtcatggtggatgccgttca | VTCGAATLSAERVRG |
| | caagctcaagtatgaaaactacaccagcagcttcttcatcagggacatcatcaaac | DNKEYEYSVECQEDS |
| | ctgacccacccaagaacttgcagctgaagccattaaagaattctcggcaggtgga | ACPAAEESLPIEVMV |
| | ggtcagctgggagtaccctgacacctggagtactccacattcctacttctccctgac | DAVHKLKYENYTSSFF |
| | attctgcgttcaggtccagggcaagagcaagagagaaaagaaagatagagtcttc | IRDIIKPDPPKNLQLKP |
| | acggacaagacctcagccacggtcatctgccgcaaaaatgccagcattagcgtgc | LKNSRQVEVSWEYPD |
| | gggcccaggaccgctactatagctcatcttggagcgaatgggcatctgtgccctgc | TWSTPHSYFSLTFCV |
| | agtAGAGCCgaattcCTGGAGAACGGAGGGACAAGCCTGAGCGA | QVQGKSKREKKDRVF |
| | GAAGACAGTGCTGCTGCTGGTGACCCCATTTCTGGCCGCAGCA | TDKTSATVICRKNASI |
| | TGGAGCCTGCACCCCGCTGGAACGCGTggctccggcgagggcagggg | SVRAQDRYYSSSWSE |
| | aagtcttctaacatgcggggacgtggaggaaaatcccggcccaCTCGAGGGC | WASVPCSRAEFLENG |
| | ACCAGCCTGCTGTGCTGGATGGCACTGTGCCTGCTGGGAGCAG | GTSLSEKTVLLLVTPFL |
| | ACCACGCCGATGCCGGCATCCACGTGTTCATCCTGGGCTGCTTC | AAAWSLHPAGTRGS |
| | AGCGCCGGACTTCCTAAAACAGAGGCCAACTGGGTGAACGTG | GEGRGSLLTCGDVEE |
| | ATTAGCGACCTGAAGAAGATCGAGGACCTGATCCAGAGCATG | NPGPLEGTSLLCWM |
| | CACATCGACGCCACCCTGTACACCGAGAGCGATGTGCATCCTA | ALCLLGADHADAGIH |
| | GCTGCAAGGTGACCGCCATGAAGTGCTTCCTGCTGGAGCTGCA | VFILGCFSAGLPKTEA |
| | GGTGATCAGCCTGGAGAGCGGAGATGCCAGCATTCACGACAC | NWVNVISDLKKIEDLI |
| | AGTGGAAAATCTGATCATCCTGGCCAACAACAGCCTGAGCAGC | QSMHIDATLYTESDV |
| | AACGGCAATGTGACCGAGAGCGGCTGTAAGGAGTGCGAGGA | HPSCKVTAMKCFLLEL |
| | ACTGGAGGAGAAGAACATCAAGGAGTTCCTGCAGAGCTTCGT | QVISLESGDASIHDTV |
| | GCACATCGTGCAGATGTTCATCAACACCAGCAGAGCCAGGGCC | ENLIILANNSLSSNGN |
| | GAGTTCcttgaaaatggtgggacatccttatcagagaaaacagttcttctgctgg | VTESGCKECEELEEKN |
| | tgactccatttctggcagcagcctggagccttcatccc (SEQ ID NO: 628) | IKEFLQSFVHIVQMFI |
| | | NTSRARAEFLENGGT |
| | | SLSEKTVLLLVTPFLAA |
| | | AWSLHP (SEQ ID |
| | | NO: 641) |
| SP1-IL-7- | gctgtgatggcccctagaaccctgctgctgctgctgagcggcgccctggccctgac | AVMAPRTLLLLLSGAL |
| Anchoring | acagacctgggccgattgtgatattgaaggtaaagatggcaaacaatatgagagt | ALTQTWADCDIEGKD |
| structure 1- | gttctaatggtcagcatcgatcaattattggacagcatgaaagaaattggtagcaa | GKQYESVLMVSIDQL |
| P2A-SP2-IL-15- | ttgcctgaataatgaatttaacttttttaaaagacatatctgtgatgctaataagga | LDSMKEIGSNCLNNE |
| Anchoring | aggtatgtttttattccgtgctgctcgcaagttgaggcaatttcttaaaatgaatag | FNFFKRHICDANKEG |
| structure 2 | cactggtgattttgatctccacttattaaaagtttcagaaggcacaacaatactgtt | MFLFRAARKLRQFLK |
| | gaactgcactggccaggttaaaggaagaaaaccagctgccctgggtgaagcccaacc | MNSTGDFDLHLLKVS |
| | aacaaagagtttggaagaaaataaatctttaaaggaacagaaaaaactgaatga | EGTTILLNCTGQVKGR |
| | cttgtgtttcctaaagagactattacaagagataaaaacttgttggaataaaatttt | KPAALGEAQPTKSLEE |
| | gatgggcactaaagaacacgaattccttgaaaatggtgggacatccttatcagag | NKSLKEQKKLNDLCFL |
| | aaaacagttcttctgctggtgactccatttctggcagcagcctggagccttcatccc | KRLLQEIKTCWNKIL |
| | ggatctggagcaacaaacttctcactactcaaacaagcaggtgacgtggaggagaa | MGTKEHEFLENGGTS |
| | tccggggcccggcaccagcctgctgtgctggatggcactgtgcctgctgggagcag | LSEKTVLLLVTPFLAAA |
| | accacgccgatgccggcatccacgtgttcatcctgggctgcttcagcgccggacttc | WSLHPGSGATNFSLL |
| | ctaaaacagaggccaactgggtgaacgtgattagcgacctgaagaagatcgagg | KQAGDVEENPGPGT |
| | acctgatccagagcatgcacatcgacgccaccctgtacaccgagagcgatgtgcat | SLLCWMALCLLGADH |
| | cctagctgcaaggtgaccgccatgaagtgcttcctgctggagctgcaggtgatcag | ADAGIHVFILGCFSAG |
| | cctggagagcggagatgccagcattcacgacacagtggaaaatctgatcatcctg | LPKTEANWVNVISDL |
| | gccaacaacagcctgagcagcaacggcaatgtgaccgagagcggctgtaaggag | KKIEDLIQSMHIDATL |
| | tgcgaggaactggaggagaagaacatcaaggagttcctgcagagcttcgtgcaca | YTESDVHPSCKVTAM |
| | tcgtgcagatgttcatcaacaccagcagagccgagttcctggagaacggtggcacc | KCFLLELQVISLESGDA |
| | agcctttccgagaagaccgtcttgctgctggtgactccttttctggccgcggcttgg | SIHDTVENLIILANNSL |
| | tctctccacccc (SEQ ID NO: 629) | SSNGNVTESGCKECE |
| | | ELEEKNIKEFLQSFVHI |
| | | VQMFINTSRAEFLEN |
| | | GGTSLSEKTVLLLVTP |
| | | FLAAAWSLHP (SEQ |
| | | ID NO: 642) |
| IL-12p40-Lr1- | gctgtgatggcccctagaaccctgctgctgctgctgagcggcgccctggccctgac | AVMAPRTLLLLLSGAL |
| Ar1-E2A-IL-15- | acagacctgggccatatgggaactgaagaaagatgtttatgtcgtagaattggatt | ALTQTWAIWELKKDV |
| Lr1-Ar2 | ggtatccggatgcccctggagaaatggtggtcctcacctgtgacacccctgaagaa | YVVELDWYPDAPGE |
| | gatggtatcacctggaccttggaccagagcagtgaggtcttaggctctggcaaaac | MVVLTCDTPEEDGIT |
| | cctgaccatccaagtcaaagagtttggagatgctggccagtacacctgtcacaaag | WTLDQSSEVLGSGKT |
| | gaggcgaggttctaagccattcgctcctgctgcttcacaaaaaggaagatggaatt | LTIQVKEFGDAGQYT |
| | tggtccactgatattttaaaggaccagaaagaacccaaaaataagacctttctaag | CHKGGEVLSHSLLLLH |
| | atgcgaggccaagaattattctggacgtttcacctgctggtggctgacgacaatca | KKEDGIWSTDILKDQ |
| | gtactgatttgacattcagtgtcaaaagcagcagaggctcttctgacccccaaggg | KEPKNKTFLRCEAKNY |
| | gtgacgtgcggagctgctacactctctgcagagagagtcagaggggacaacaagg | SGRFTCWWLTTISTD |
| | agtatgagtactcagtggagtgccaggaggacagtgcctgcccagctgctgagga | LTFSVKSSRGSSDPQG |
| | gagtctgcccattgaggtcatggtggatgccgttcacaagctcaagtatgaaaact | VTCGAATLSAERVRG |
| | acaccagcagcttcttcatcagggacatcatcaaacctgacccacccaagaacttg | DNKEYEYSVECQEDS |
| | cagctgaagccattaaagaattctcggcaggtggaggtcagctgggagtaccctg | ACPAAEESLPIEVMV |
| | acacctggagtactccacattcctacttctccctgacattctgcgttcaggtccagg | DAVHKLKYENYTSSFF |
| | gcaagagcaagagagaaaagaaagatagagtcttcacggacaagacctcagccac | IRDIIKPDPPKNLQLKP |
| | ggtcatctgccgcaaaaatgccagcattagcgtgcgggcccaggaccgctactata | LKNSRQVEVSWEYPD |
| | gctcatcttggagcgaatgggcatctgtgccctgcagttctggaggaggaggatct | TWSTPHSYFSLTFCV |
| | ggcggaggaggaagtggaggaggaggctctggaggaggcggatctggaggagg | QVQGKSKREKKDRVF |
| | aagcctgcagatctacatctgggctcctctggctggcacctgcggagtgctgctgct | TDKTSATVICRKNASI |
| | gtctctggtgattactgggagcgggcagtgcactaactatgccctgctgaagctggc | SVRAQDRYYSSSWSE |
| | cggggacgtggaatctaaccccggacctggaacctccctgctgtgttggatggccc | WASVPCSSGGGGSG |
| | tgtgcctgctgggagctgatcacgctgacgctggaatccacgtgtttatcctgggct | GGGSGGGGSGGGGS |
| | gctttagcgccggcctgcctaaaaccgaagccaactgggtgaacgtgatctccgac | GGGSLQIYIWAPLAG |
| | ctgaagaagatcgaagacctgattcagtctatgcacatcgacgccaccctgtacac | TCGVLLLSLVITGSGQ |
| | cgagagtgacgtgcacccctcctgcaaagtgacagccatgaagtgcttcctgctgg | CTNYALLKLAGDVES |
| | agctgcaggtgatcagcctggaaagcggcgacgctagtatccacgacaccgtgga | NPGPGTSLLCWMAL |
| | gaacctgattattctggctaacaactccctgagcagtaacggaaacgtgaccgagt | CLLGADHADAGIHVFI |
| | ccggctgcaaagagtgcgaggagctggaggagaagaacattaaggagttcctgc | LGCFSAGLPKTEANW |
| | agagcttcgtgcacatcgtgcagatgttcatcaacaccagcagagccagtggcggc | VNVISDLKKIEDLIQS |
| | ggaggatctggaggcggaggaagcggaggaggaggatcaggaggaggaggaa | MHIDATLYTESDVHP |
| | gcggcggaggatcactgcagctgctgcctagttgggccatcaccctgatcagcgtg | SCKVTAMKCFLLELQ |
| | aacggaatcttcgtgatctgttgtctgacatactgttttgctcccaggtgcagagag | VISLESGDASIHDTVE |
| | aggagaaggaacgagcggctgagaagagaatccgtgcggcctgtg (SEQ ID | NLIILANNSLSSNGNV |
| | NO: 630) | TESGCKECEELEEKNI |
| | | KEFLQSFVHIVQMFIN |
| | | TSRASGGGGSGGGG |
| | | SGGGGSGGGGSGGG |
| | | SLQLLPSWAITLISVN |
| | | GIFVICCLTYCFAPRCR |
| | | ERRRNERLRRESVRP |
| | | V (SEQ ID NO: 643) |
| IL-12p40-Lr1- | gctgtgatggcccctagaaccctgctgctgctgctgagcggcgccctggccctgac | AVMAPRTLLLLLSGAL |
| Ar1-F2A-IL-15- | acagacctgggccatatgggaactgaagaaagatgtttatgtcgtagaattggatt | ALTQTWAIWELKKDV |
| Lr1-Ar2 | ggtatccggatgcccctggagaaatggtggtcctcacctgtgacacccctgaagaa | YVVELDWYPDAPGE |
| | gatggtatcacctggaccttggaccagagcagtgaggtcttaggctctggcaaaac | MVVLTCDTPEEDGIT |
| | cctgaccatccaagtcaaagagtttggagatgctggccagtacacctgtcacaaag | WTLDQSSEVLGSGKT |
| | gaggcgaggttctaagccattcgctcctgctgcttcacaaaaaggaagatggaatt | LTIQVKEFGDAGQYT |
| | tggtccactgatattttaaaggaccagaaagaacccaaaaataagacctttctaag | CHKGGEVLSHSLLLLH |
| | atgcgaggccaagaattattctggacgtttcacctgctggtggctgacgacaatca | KKEDGIWSTDILKDQ |
| | gtactgatttgacattcagtgtcaaaagcagcagaggctcttctgacccccaaggg | KEPKNKTFLRCEAKNY |
| | gtgacgtgcggagctgctacactctctgcagagagagtcagaggggacaacaagg | SGRFTCWWLTTISTD |
| | agtatgagtactcagtggagtgccaggaggacagtgcctgcccagctgctgagga | LTFSVKSSRGSSDPQG |
| | gagtctgcccattgaggtcatggtggatgccgttcacaagctcaagtatgaaaact | VTCGAATLSAERVRG |
| | acaccagcagcttcttcatcagggacatcatcaaacctgacccacccaagaacttg | DNKEYEYSVECQEDS |
| | cagctgaagccattaaagaattctcggcaggtggaggtcagctgggagtaccctg | ACPAAEESLPIEVMV |
| | acacctggagtactccacattcctacttctccctgacattctgcgttcaggtccagg | DAVHKLKYENYTSSFF |
| | gcaagagcaagagagaaaagaaagatagagtcttcacggacaagacctcagccac | IRDIIKPDPPKNLQLKP |
| | ggtcatctgccgcaaaaatgccagcattagcgtgcgggcccaggaccgctactata | LKNSRQVEVSWEYPD |
| | gctcatcttggagcgaatgggcatctgtgccctgcagttctggaggaggaggatct | TWSTPHSYFSLTFCV |
| | ggcggaggaggaagtggaggaggaggctctggaggaggcggatctggaggagg | QVQGKSKREKKDRVF |
| | gagtctgcagatctacatctgggctcctctggctggcacctgcggagtgctgctgct | TDKTSATVICRKNASI |
| | gtctctggtgattactagggccaagaggagcggaagcggggctacaaacttcagt | SVRAQDRYYSSSWSE |
| | ctgctgaagcaggctggcgacgtggaggaaaaccctggacctggaaccagcctgc | WASVPCSSGGGGSG |
| | tgtgctggatggctctgtgcctgctgggagctgaccacgctgatgctggaatccacg | GGGSGGGGSGGGGS |
| | tgtttatcctgggatgcttctctgccgggctgcctaagaccgaggctaattgggtga | GGGSLQIYIWAPLAG |
| | acgtgatcagcgacctgaaaaagatcgaagatctgatccagtctatgcacatcgac | TCGVLLLSLVITRAKRS |
| | gccactctgtacaccgagtccgacgtgcacccctcttgcaaggtgaccgctatgaa | GSGATNFSLLKQAGD |
| | gtgcttcctgctggagctgcaggtgatcagcctggagagcggagacgctagcatcc | VEENPGPGTSLLCW |
| | acgacacagtggaaaacctgattatcctggctaacaacagcctgagcagtaacgg | MALCLLGADHADAGI |
| | aaacgtgaccgagtccggctgcaaagagtgcgaggagctggaagagaagaacat | HVFILGCFSAGLPKTE |
| | taaggagttcctgcagagcttcgtgcacatcgtgcagatgttcatcaacaccagca | ANWVNVISDLKKIED |
| | gagccagtggcgggggaggatctggaggcggaggatctgggggaggaggaagc | LIQSMHIDATLYTESD |
| | ggaggaggagggagcggaggaggcagcctgcagctgctgccttcttgggctatca | VHPSCKVTAMKCFLL |
| | ccctgatcagcgtgaacggaatctttgtgatctgttgtctgacttattgtttcgccc | ELQVISLESGDASIHD |
| | ccagatgcagagagaggagaaggaatgagagactgagaagagaatcagtgaggcc | TVENLIILANNSLSSN |
| | cgtg (SEQ ID NO: 631) | GNVTESGCKECEELEE |
| | | KNIKEFLQSFVHIVQ |
| | | MFINTSRASGGGGSG |
| | | GGGSGGGGSGGGGS |
| | | GGGSLQLLPSWAITLI |
| | | SVNGIFVICCLTYCFAP |
| | | RCRERRRNERLRRES |
| | | VRPV (SEQ ID NO: |
| | | 644) |
| IL-12p40-Lr1- | gctgtgatggcccctagaaccctgctgctgctgctgagcggcgccctggccctgac | AVMAPRTLLLLLSGAL |
| Ar1-P2A-IL-15- | acagacctgggccatatgggaactgaagaaagatgtttatgtcgtagaattggatt | ALTQTWAIWELKKDV |
| Lr1-Ar2 | ggtatccggatgcccctggagaaatggtggtcctcacctgtgacacccctgaagaa | YVVELDWYPDAPGE |
| | gatggtatcacctggaccttggaccagagcagtgaggtcttaggctctggcaaaac | MVVLTCDTPEEDGIT |
| | cctgaccatccaagtcaaagagtttggagatgctggccagtacacctgtcacaaag | WTLDQSSEVLGSGKT |
| | gaggcgaggttctaagccattcgctcctgctgcttcacaaaaaggaagatggaatt | LTIQVKEFGDAGQYT |
| | tggtccactgatattttaaaggaccagaaagaacccaaaaataagacctttctaag | CHKGGEVLSHSLLLLH |
| | atgcgaggccaagaattattctggacgtttcacctgctggtggctgacgacaatca | KKEDGIWSTDILKDQ |
| | gtactgatttgacattcagtgtcaaaagcagcagaggctcttctgacccccaaggg | KEPKNKTFLRCEAKNY |
| | gtgacgtgcggagctgctacactctctgcagagagagtcagaggggacaacaagg | SGRFTCWWLTTISTD |
| | agtatgagtactcagtggagtgccaggaggacagtgcctgcccagctgctgagga | LTFSVKSSRGSSDPQG |
| | gagtctgcccattgaggtcatggtggatgccgttcacaagctcaagtatgaaaact | VTCGAATLSAERVRG |
| | acaccagcagcttcttcatcagggacatcatcaaacctgacccacccaagaacttg | DNKEYEYSVECQEDS |
| | cagctgaagccattaaagaattctcggcaggtggaggtcagctgggagtaccctg | ACPAAEESLPIEVMV |
| | acacctggagtactccacattcctacttctccctgacattctgcgttcaggtccagg | DAVHKLKYENYTSSFF |
| | gcaagagcaagagagaaaagaaagatagagtcttcacggacaagacctcagccac | IRDIIKPDPPKNLQLKP |
| | ggtcatctgccgcaaaaatgccagcattagcgtgcgggcccaggaccgctactata | LKNSRQVEVSWEYPD |
| | gctcatcttggagcgaatgggcatctgtgccctgcagttctggaggaggaggatct | TWSTPHSYFSLTFCV |
| | ggcggaggaggaagtggaggaggaggctctggaggaggcggatctggaggagg | QVQGKSKREKKDRVF |
| | gagtctgcagatctacatctgggctcctctggctggcacctgcggagtgctgctgct | TDKTSATVICRKNASI |
| | gtctctggtgattactgggtccggcgctacaaacttcagtctgctgaagcaggctgg | SVRAQDRYYSSSWSE |
| | cgatgtggaggaaaaccctggccctggaacctccctgctgtgttggatggctctgtg | WASVPCSSGGGGSG |
| | tctgctgggggctgaccacgctgatgctggaatccacgtgttcatcctgggctgctt | GGGSGGGGSGGGGS |
| | tagcgccggcctgcctaaaaccgaggccaattgggtgaacgtgatttctgacctgaa | GGGSLQIYIWAPLAG |
| | gaagatcgaggacctgattcagagtatgcatattgacgccaccctgtacaccgaga | TCGVLLLSLVITGSGA |
| | gtgacgtgcaccctagctgcaaagtgacagccatgaagtgcttcctgctggagctg | TNFSLLKQAGDVEEN |
| | caggtgatcagcctggaaagcggcgacgctagcatccacgacaccgtggaaaac | PGPGTSLLCWMALCL |
| | ctgattatcctggccaacaactccctgagcagtaacggaaacgtgaccgagtccgg | LGADHADAGIHVFIL |
| | ctgcaaagagtgcgaggagctggaagagaaaaacattaaagaattcctgcagtcc | GCFSAGLPKTEANWV |
| | ttcgtgcacatcgtgcagatgttcatcaacaccagcagagccagtggcgggggag | NVISDLKKIEDLIQSM |
| | gatctggaggcggaggatctgggggaggaggaagcggaggaggagggagcgga | HIDATLYTESDVHPSC |
| | ggaggcagcctgcagctgctgccttcttgggctatcaccctgatcagcgtgaacgg | KVTAMKCFLLELQVIS |
| | aatctttgtgatctgttgtctgacttattgtttcgcccccagatgcagagagaggag | LESGDASIHDTVENLII |
| | aaggaatgagagactgagaagagaatcagtgaggcccgtg (SEQ ID NO: | LANNSLSSNGNVTES |
| | 632) | GCKECEELEEKNIKEFL |
| | | QSFVHIVQMFINTSR |
| | | ASGGGGSGGGGSGG |
| | | GGSGGGGSGGGSLQ |
| | | LLPSWAITLISVNGIFV |
| | | ICCLTYCFAPRCRERR |
| | | RNERLRRESVRPV |
| | | (SEQ ID NO: 645) |
| IL-12p40-Lr1- | gctgtgatggcccctagaaccctgctgctgctgctgagcggcgccctggccctgac | AVMAPRTLLLLLSGAL |
| Ar1-T2A-IL-15- | acagacctgggccatatgggaactgaagaaagatgtttatgtcgtagaattggatt | ALTQTWAIWELKKDV |
| Lr1-Ar2 | ggtatccggatgcccctggagaaatggtggtcctcacctgtgacacccctgaagaa | YVVELDWYPDAPGE |
| | gatggtatcacctggaccttggaccagagcagtgaggtcttaggctctggcaaaac | MVVLTCDTPEEDGIT |
| | cctgaccatccaagtcaaagagtttggagatgctggccagtacacctgtcacaaag | WTLDQSSEVLGSGKT |
| | gaggcgaggttctaagccattcgctcctgctgcttcacaaaaaggaagatggaatt | LTIQVKEFGDAGQYT |
| | tggtccactgatattttaaaggaccagaaagaacccaaaaataagacctttctaag | CHKGGEVLSHSLLLLH |
| | atgcgaggccaagaattattctggacgtttcacctgctggtggctgacgacaatca | KKEDGIWSTDILKDQ |
| | gtactgatttgacattcagtgtcaaaagcagcagaggctcttctgacccccaaggg | KEPKNKTFLRCEAKNY |
| | gtgacgtgcggagctgctacactctctgcagagagagtcagaggggacaacaagg | SGRFTCWWLTTISTD |
| | agtatgagtactcagtggagtgccaggaggacagtgcctgcccagctgctgagga | LTFSVKSSRGSSDPQG |
| | gagtctgcccattgaggtcatggtggatgccgttcacaagctcaagtatgaaaact | VTCGAATLSAERVRG |
| | acaccagcagcttcttcatcagggacatcatcaaacctgacccacccaagaacttg | DNKEYEYSVECQEDS |
| | cagctgaagccattaaagaattctcggcaggtggaggtcagctgggagtaccctg | ACPAAEESLPIEVMV |
| | acacctggagtactccacattcctacttctccctgacattctgcgttcaggtccagg | DAVHKLKYENYTSSFF |
| | gcaagagcaagagagaaaagaaagatagagtcttcacggacaagacctcagccac | IRDIIKPDPPKNLQLKP |
| | ggtcatctgccgcaaaaatgccagcattagcgtgcgggcccaggaccgctactata | LKNSRQVEVSWEYPD |
| | gctcatcttggagcgaatgggcatctgtgccctgcagttctggaggaggaggatct | TWSTPHSYFSLTFCV |
| | ggcggaggaggaagtggaggaggaggctctggaggaggcggatctggaggagg | QVQGKSKREKKDRVF |
| | gagtctgcagatctacatctgggctcctctggctggcacctgcggagtgctgctgct | TDKTSATVICRKNASI |
| | gtctctggtgattactgggtccggcgaaggaagaggctctctgctgacatgcgggg | SVRAQDRYYSSSWSE |
| | atgtggaagagaatcccggccctggaacctccctgctgtgttggatggccctgtgtc | WASVPCSSGGGGSG |
| | tgctgggggctgatcacgctgatgccggaatccacgtgtttatcctgggctgcttca | GGGSGGGGSGGGGS |
| | gcgccgggctgcctaagacagaggctaactgggtgaacgtgatcagcgacctgaa | GGGSLQIYIWAPLAG |
| | aaagatcgaggacctgattcagagtatgcatatcgacgccaccctgtacaccgag | TCGVLLLSLVITGSGE |
| | agcgacgtgcatccttcctgcaaggtgacagccatgaagtgtttcctgctggagctg | GRGSLLTCGDVEENP |
| | caggtgatcagcctggaaagcggcgacgctagcatccacgacacagtggagaac | GPGTSLLCWMALCLL |
| | ctgatcatcctggccaacaacagcctgagcagcaacggaaacgtgaccgagtccg | GADHADAGIHVFILG |
| | gctgcaaagaatgtgaggaactggaggaaaagaacattaaggagttcctgcaga | CFSAGLPKTEANWVN |
| | gcttcgtgcacatcgtgcagatgttcatcaacaccagcagagccagtggcggggg | VISDLKKIEDLIQSMHI |
| | aggatctggaggcggaggatctgggggaggaggaagcggaggaggagggagcg | DATLYTESDVHPSCKV |
| | gaggaggcagcctgcagctgctgccttcttgggctatcaccctgatcagcgtgaac | TAMKCFLLELQVISLE |
| | ggaatctttgtgatctgttgtctgacttattgtttcgcccccagatgcagagagagg | SGDASIHDTVENLIILA |
| | agaaggaatgagagactgagaagagaatcagtgaggcccgtg (SEQ ID NO: | NNSLSSNGNVTESGC |
| | 633) | KECEELEEKNIKEFLQS |
| | | FVHIVQMFINTSRAS |
| | | GGGGSGGGGSGGG |
| | | GSGGGGSGGGSLQLL |
| | | PSWAITLISVNGIFVIC |
| | | CLTYCFAPRCRERRRN |
| | | ERLRRESVRPV (SEQ |
| | | ID NO: 646) |
| IL-12p40-Lr1- | gctgtgatggcccctagaaccctgctgctgctgctgagcggcgccctggccctgac | AVMAPRTLLLLLSGAL |
| Ar2-E2A-IL-15- | acagacctgggccatatgggaactgaagaaagatgtttatgtcgtagaattggatt | ALTQTWAIWELKKDV |
| Lr1-Ar1 | ggtatccggatgcccctggagaaatggtggtcctcacctgtgacacccctgaagaa | YVVELDWYPDAPGE |
| | gatggtatcacctggaccttggaccagagcagtgaggtcttaggctctggcaaaac | MVVLTCDTPEEDGIT |
| | cctgaccatccaagtcaaagagtttggagatgctggccagtacacctgtcacaaag | WTLDQSSEVLGSGKT |
| | gaggcgaggttctaagccattcgctcctgctgcttcacaaaaaggaagatggaatt | LTIQVKEFGDAGQYT |
| | tggtccactgatattttaaaggaccagaaagaacccaaaaataagacctttctaag | CHKGGEVLSHSLLLLH |
| | atgcgaggccaagaattattctggacgtttcacctgctggtggctgacgacaatca | KKEDGIWSTDILKDQ |
| | gtactgatttgacattcagtgtcaaaagcagcagaggctcttctgacccccaaggg | KEPKNKTFLRCEAKNY |
| | gtgacgtgcggagctgctacactctctgcagagagagtcagaggggacaacaagg | SGRFTCWWLTTISTD |
| | agtatgagtactcagtggagtgccaggaggacagtgcctgcccagctgctgagga | LTFSVKSSRGSSDPQG |
| | gagtctgcccattgaggtcatggtggatgccgttcacaagctcaagtatgaaaact | VTCGAATLSAERVRG |
| | acaccagcagcttcttcatcagggacatcatcaaacctgacccacccaagaacttg | DNKEYEYSVECQEDS |
| | cagctgaagccattaaagaattctcggcaggtggaggtcagctgggagtaccctg | ACPAAEESLPIEVMV |
| | acacctggagtactccacattcctacttctccctgacattctgcgttcaggtccagg | DAVHKLKYENYTSSFF |
| | gcaagagcaagagagaaaagaaagatagagtcttcacggacaagacctcagccac | IRDIIKPDPPKNLQLKP |
| | ggtcatctgccgcaaaaatgccagcattagcgtgcgggcccaggaccgctactata | LKNSRQVEVSWEYPD |
| | gctcatcttggagcgaatgggcatctgtgccctgcagttctggaggaggaggatct | TWSTPHSYFSLTFCV |
| | ggcggaggaggaagtggaggaggaggctctggaggaggcggatctggaggagg | QVQGKSKREKKDRVF |
| | gagtctgcagctgctgccaagttgggctatcacactgattagcgtgaacggaatctt | TDKTSATVICRKNASI |
| | tgtgatctgttgcctgacatactgctttgctcctaggtgcagggagaggagaagga | SVRAQDRYYSSSWSE |
| | atgagagactgagaagagagtccgtgaggcccgtgggaagcggacagtgtacaa | WASVPCSSGGGGSG |
| | actacgccctgctgaagctggccggcgatgtggaatccaaccccggacctggaac | GGGSGGGGSGGGGS |
| | aagcctgctgtgttggatggccctgtgtctgctgggcgctgatcatgctgacgccgg | GGGSLQLLPSWAITLI |
| | aatccacgtgtttatcctggggtgctttagcgccggcctgcctaaaaccgaggccaa | SVNGIFVICCLTYCFAP |
| | ttgggtgaacgtgatcagcgacctgaaaaagatcgaggacctgattcagagtatg | RCRERRRNERLRRES |
| | catatcgacgccaccctgtacaccgagagtgacgtgcacccctcctgcaaagtgac | VRPVGSGQCTNYALL |
| | cgctatgaagtgcttcctgctggagctgcaggtgatcagcctggaaagcggcgacg | KLAGDVESNPGPGTS |
| | ctagcatccacgacaccgtggaaaacctgatcatcctggccaacaactccctgagc | LLCWMALCLLGADH |
| | agcaacggaaacgtgaccgagtccggctgcaaagaatgtgaggaactggaggaa | ADAGIHVFILGCFSAG |
| | aagaacatcaaagaattcctgcagtctttcgtgcacatcgtgcagatgttcatcaac | LPKTEANWVNVISDL |
| | accagcagagccagcggcgggggaggatctggaggcggaggatctgggggagg | KKIEDLIQSMHIDATL |
| | aggaagcggaggaggagggagcggaggaggcagcctgcagatctatatttgggc | YTESDVHPSCKVTAM |
| | cccactggccggcacctgcggagtgctgctgctgtctctggtgattaca (SEQ | KCFLLELQVISLESGDA |
| | ID NO: 634) | SIHDTVENLIILANNSL |
| | | SSNGNVTESGCKECE |
| | | ELEEKNIKEFLQSFVHI |
| | | VQMFINTSRASGGG |
| | | GSGGGGSGGGGSGG |
| | | GGSGGGSLQIYIWAP |
| | | LAGTCGVLLLSLVIT |
| | | (SEQ ID NO: 647) |
| IL-12p40-Lr1- | gctgtgatggcccctagaaccctgctgctgctgctgagcggcgccctggccctgac | AVMAPRTLLLLLSGAL |
| Ar2-E2A-IL-15- | acagacctgggccatatgggaactgaagaaagatgtttatgtcgtagaattggatt | ALTQTWAIWELKKDV |
| Lr1-Ar2 | ggtatccggatgcccctggagaaatggtggtcctcacctgtgacacccctgaagaa | YVVELDWYPDAPGE |
| | gatggtatcacctggaccttggaccagagcagtgaggtcttaggctctggcaaaac | MVVLTCDTPEEDGIT |
| | cctgaccatccaagtcaaagagtttggagatgctggccagtacacctgtcacaaag | WTLDQSSEVLGSGKT |
| | gaggcgaggttctaagccattcgctcctgctgcttcacaaaaaggaagatggaatt | LTIQVKEFGDAGQYT |
| | tggtccactgatattttaaaggaccagaaagaacccaaaaataagacctttctaag | CHKGGEVLSHSLLLLH |
| | atgcgaggccaagaattattctggacgtttcacctgctggtggctgacgacaatca | KKEDGIWSTDILKDQ |
| | gtactgatttgacattcagtgtcaaaagcagcagaggctcttctgacccccaaggg | KEPKNKTFLRCEAKNY |
| | gtgacgtgcggagctgctacactctctgcagagagagtcagaggggacaacaagg | SGRFTCWWLTTISTD |
| | agtatgagtactcagtggagtgccaggaggacagtgcctgcccagctgctgagga | LTFSVKSSRGSSDPQG |
| | gagtctgcccattgaggtcatggtggatgccgttcacaagctcaagtatgaaaact | VTCGAATLSAERVRG |
| | acaccagcagcttcttcatcagggacatcatcaaacctgacccacccaagaacttg | DNKEYEYSVECQEDS |
| | cagctgaagccattaaagaattctcggcaggtggaggtcagctgggagtaccctg | ACPAAEESLPIEVMV |
| | acacctggagtactccacattcctacttctccctgacattctgcgttcaggtccagg | DAVHKLKYENYTSSFF |
| | gcaagagcaagagagaaaagaaagatagagtcttcacggacaagacctcagccac | IRDIIKPDPPKNLQLKP |
| | ggtcatctgccgcaaaaatgccagcattagcgtgcgggcccaggaccgctactata | LKNSRQVEVSWEYPD |
| | gctcatcttggagcgaatgggcatctgtgccctgcagttctggaggaggaggatct | TWSTPHSYFSLTFCV |
| | ggcggaggaggaagtggaggaggaggctctggaggaggcggatctggaggagg | QVQGKSKREKKDRVF |
| | gagtctgcagctgctgccaagttgggctatcacactgattagcgtgaacggaatctt | TDKTSATVICRKNASI |
| | tgtgatctgttgcctgacatactgctttgctcctaggtgcagggagaggagaagga | SVRAQDRYYSSSWSE |
| | atgagagactgagaagagagtccgtgaggcccgtgggaagcggacagtgtacaa | WASVPCSSGGGGSG |
| | actacgccctgctgaagctggccggcgatgtggaatccaaccccggacctggaac | GGGSGGGGSGGGGS |
| | aagcctgctgtgttggatggccctgtgtctgctgggcgctgatcatgctgacgccgg | GGGSLQLLPSWAITLI |
| | aatccacgtgtttatcctggggtgctttagcgccggcctgcctaaaaccgaggccaa | SVNGIFVICCLTYCFAP |
| | ttgggtgaacgtgatcagcgacctgaaaaagatcgaggacctgattcagagtatg | RCRERRRNERLRRES |
| | catatcgacgccaccctgtacaccgagagtgacgtgcacccctcctgcaaagtgac | VRPVGSGQCTNYALL |
| | cgctatgaagtgcttcctgctggagctgcaggtgatcagcctggaaagcggcgacg | KLAGDVESNPGPGTS |
| | ctagcatccacgacaccgtggaaaacctgatcatcctggccaacaactccctgagc | LLCWMALCLLGADH |
| | agcaacggaaacgtgaccgagtccggctgcaaagaatgtgaggaactggaggaa | ADAGIHVFILGCFSAG |
| | aagaacatcaaagaattcctgcagtctttcgtgcacatcgtgcagatgttcatcaac | LPKTEANWVNVISDL |
| | accagcagagccagcggcgggggaggatctggaggcggaggatctgggggagg | KKIEDLIQSMHIDATL |
| | aggaagcggaggaggagggagcggaggaggcagcctgcagctgctgccctcttg | YTESDVHPSCKVTAM |
| | ggctatcaccctgatcagcgtgaacgggatctttgtgatttgttgtctgacttactg | KCFLLELQVISLESGDA |
| | tttcgcccccagatgcagagagagaaggagaaacgagagactgaggagagaaagc | SIHDTVENLIILANNSL |
| | gtgaggcccgtg (SEQ ID NO: 635) | SSNGNVTESGCKECE |
| | | ELEEKNIKEFLQSFVHI |
| | | VQMFINTSRASGGG |
| | | GSGGGGSGGGGSGG |
| | | GGSGGGSLQLLPSW |
| | | AITLISVNGIFVICCLTY |
| | | CFAPRCRERRRNERL |
| | | RRESVRPV (SEQ ID |
| | | NO: 648) |
| IL-12p40-Lr1- | gctgtgatggcccctagaaccctgctgctgctgctgagcggcgccctggccctgac | AVMAPRTLLLLLSGAL |
| Ar2-T2A-IL-15- | acagacctgggccatatgggaactgaagaaagatgtttatgtcgtagaattggatt | ALTQTWAIWELKKDV |
| Lr1-Ar1 | ggtatccggatgcccctggagaaatggtggtcctcacctgtgacacccctgaagaa | YVVELDWYPDAPGE |
| | gatggtatcacctggaccttggaccagagcagtgaggtcttaggctctggcaaaac | MVVLTCDTPEEDGIT |
| | cctgaccatccaagtcaaagagtttggagatgctggccagtacacctgtcacaaag | WTLDQSSEVLGSGKT |
| | gaggcgaggttctaagccattcgctcctgctgcttcacaaaaaggaagatggaatt | LTIQVKEFGDAGQYT |
| | tggtccactgatattttaaaggaccagaaagaacccaaaaataagacctttctaag | CHKGGEVLSHSLLLLH |
| | atgcgaggccaagaattattctggacgtttcacctgctggtggctgacgacaatca | KKEDGIWSTDILKDQ |
| | gtactgatttgacattcagtgtcaaaagcagcagaggctcttctgacccccaaggg | KEPKNKTFLRCEAKNY |
| | gtgacgtgcggagctgctacactctctgcagagagagtcagaggggacaacaagg | SGRFTCWWLTTISTD |
| | agtatgagtactcagtggagtgccaggaggacagtgcctgcccagctgctgagga | LTFSVKSSRGSSDPQG |
| | gagtctgcccattgaggtcatggtggatgccgttcacaagctcaagtatgaaaact | VTCGAATLSAERVRG |
| | acaccagcagcttcttcatcagggacatcatcaaacctgacccacccaagaacttg | DNKEYEYSVECQEDS |
| | cagctgaagccattaaagaattctcggcaggtggaggtcagctgggagtaccctg | ACPAAEESLPIEVMV |
| | acacctggagtactccacattcctacttctccctgacattctgcgttcaggtccagg | DAVHKLKYENYTSSFF |
| | gcaagagcaagagagaaaagaaagatagagtcttcacggacaagacctcagccac | IRDIIKPDPPKNLQLKP |
| | ggtcatctgccgcaaaaatgccagcattagcgtgcgggcccaggaccgctactata | LKNSRQVEVSWEYPD |
| | gctcatcttggagcgaatgggcatctgtgccctgcagttctggaggaggaggatct | TWSTPHSYFSLTFCV |
| | ggcggaggaggaagtggaggaggaggctctggaggaggcggatctggaggagg | QVQGKSKREKKDRVF |
| | gagtctgcagctgctgccaagttgggctatcacactgattagcgtgaacggaatctt | TDKTSATVICRKNASI |
| | tgtgatctgttgcctgacatactgctttgctcctaggtgcagggagaggagaagga | SVRAQDRYYSSSWSE |
| | atgagagactgagaagagagtccgtgaggcccgtgggaagcggagaaggaaga | WASVPCSSGGGGSG |
| | ggaagcctgctgacctgcggggatgtggaagagaaccctggcccaggaacaagc | GGGSGGGGSGGGGS |
| | ctgctgtgctggatggccctgtgcctgctgggagctgatcatgctgacgccggaatc | GGGSLQLLPSWAITLI |
| | catgtgtttatcctgggatgcttcagcgccggactgcctaaaactgaggccaactgg | SVNGIFVICCLTYCFAP |
| | gtgaacgtgatttctgacctgaaaaagatcgaagacctgattcagagcatgcacat | RCRERRRNERLRRES |
| | cgacgccaccctgtacactgagtccgacgtgcacccctcctgcaaagtgacagcca | VRPVGSGEGRGSLLT |
| | tgaagtgtttcctgctggagctgcaggtgatcagcctggagagcggagacgctagc | CGDVEENPGPGTSLL |
| | atccacgacacagtggaaaacctgatcatcctggctaacaacagcctgagcagca | CWMALCLLGADHAD |
| | acggaaacgtgaccgagtccggctgcaaagagtgcgaggagctggaggagaag | AGIHVFILGCFSAGLP |
| | aacattaaggagttcctgcagagcttcgtgcacatcgtgcagatgttcatcaacacc | KTEANWVNVISDLKKI |
| | agcagagccagtggcgggggaggatctggaggcggaggatctgggggaggagg | EDLIQSMHIDATLYTE |
| | aagcggaggaggagggagcggaggaggcagcctgcagatctatatttgggcccc | SDVHPSCKVTAMKCF |
| | cctggccggcacatgcggagtgctgctgctgtctctggtgattaca (SEQ ID | LLELQVISLESGDASIH |
| | NO: 636) | DTVENLIILANNSLSS |
| | | NGNVTESGCKECEEL |
| | | EEKNIKEFLQSFVHIV |
| | | QMFINTSRASGGGGS |
| | | GGGGSGGGGSGGG |
| | | GSGGGSLQIYIWAPL |
| | | AGTCGVLLLSLVIT |
| | | (SEQ ID NO: 649) |
| IL-12p40-Lr1- | gctgtgatggcccctagaaccctgctgctgctgctgagcggcgccctggccctgac | AVMAPRTLLLLLSGAL |
| Ar2-T2A-IL-15- | acagacctgggccatatgggaactgaagaaagatgtttatgtcgtagaattggatt | ALTQTWAIWELKKDV |
| Lr1-Ar2 | ggtatccggatgcccctggagaaatggtggtcctcacctgtgacacccctgaagaa | YVVELDWYPDAPGE |
| | gatggtatcacctggaccttggaccagagcagtgaggtcttaggctctggcaaaac | MVVLTCDTPEEDGIT |
| | cctgaccatccaagtcaaagagtttggagatgctggccagtacacctgtcacaaag | WTLDQSSEVLGSGKT |
| | gaggcgaggttctaagccattcgctcctgctgcttcacaaaaaggaagatggaatt | LTIQVKEFGDAGQYT |
| | tggtccactgatattttaaaggaccagaaagaacccaaaaataagacctttctaag | CHKGGEVLSHSLLLLH |
| | atgcgaggccaagaattattctggacgtttcacctgctggtggctgacgacaatca | KKEDGIWSTDILKDQ |
| | gtactgatttgacattcagtgtcaaaagcagcagaggctcttctgacccccaaggg | KEPKNKTFLRCEAKNY |
| | gtgacgtgcggagctgctacactctctgcagagagagtcagaggggacaacaagg | SGRFTCWWLTTISTD |
| | agtatgagtactcagtggagtgccaggaggacagtgcctgcccagctgctgagga | LTFSVKSSRGSSDPQG |
| | gagtctgcccattgaggtcatggtggatgccgttcacaagctcaagtatgaaaact | VTCGAATLSAERVRG |
| | acaccagcagcttcttcatcagggacatcatcaaacctgacccacccaagaacttg | DNKEYEYSVECQEDS |
| | cagctgaagccattaaagaattctcggcaggtggaggtcagctgggagtaccctg | ACPAAEESLPIEVMV |
| | acacctggagtactccacattcctacttctccctgacattctgcgttcaggtccagg | DAVHKLKYENYTSSFF |
| | gcaagagcaagagagaaaagaaagatagagtcttcacggacaagacctcagccac | IRDIIKPDPPKNLQLKP |
| | ggtcatctgccgcaaaaatgccagcattagcgtgcgggcccaggaccgctactata | LKNSRQVEVSWEYPD |
| | gctcatcttggagcgaatgggcatctgtgccctgcagttctggaggaggaggatct | TWSTPHSYFSLTFCV |
| | ggcggaggaggaagtggaggaggaggctctggaggaggcggatctggaggagg | QVQGKSKREKKDRVF |
| | gagtctgcagctgctgccaagttgggctatcacactgattagcgtgaacggaatctt | TDKTSATVICRKNASI |
| | tgtgatctgttgcctgacatactgctttgctcctaggtgcagggagaggagaagga | SVRAQDRYYSSSWSE |
| | atgagagactgagaagagagtccgtgaggcccgtgggaagcggagaaggaaga | WASVPCSSGGGGSG |
| | ggaagcctgctgacctgcggggatgtggaagagaaccctggcccaggaacaagc | GGGSGGGGSGGGGS |
| | ctgctgtgctggatggccctgtgcctgctgggagctgatcatgctgacgccggaatc | GGGSLQLLPSWAITLI |
| | catgtgtttatcctgggatgcttcagcgccggactgcctaaaactgaggccaactgg | SVNGIFVICCLTYCFAP |
| | gtgaacgtgatttctgacctgaaaaagatcgaagacctgattcagagcatgcacat | RCRERRRNERLRRES |
| | cgacgccaccctgtacactgagtccgacgtgcacccctcctgcaaagtgacagcca | VRPVGSGEGRGSLLT |
| | tgaagtgtttcctgctggagctgcaggtgatcagcctggagagcggagacgctagc | CGDVEENPGPGTSLL |
| | atccacgacacagtggaaaacctgatcatcctggctaacaacagcctgagcagca | CWMALCLLGADHAD |
| | acggaaacgtgaccgagtccggctgcaaagagtgcgaggagctggaggagaag | AGIHVFILGCFSAGLP |
| | aacattaaggagttcctgcagagcttcgtgcacatcgtgcagatgttcatcaacacc | KTEANWVNVISDLKKI |
| | agcagagccagtggcgggggaggatctggaggcggaggatctgggggaggagg | EDLIQSMHIDATLYTE |
| | aagcggaggaggagggagcggaggaggcagcctgcagctgctgccctcttgggc | SDVHPSCKVTAMKCF |
| | tatcaccctgatcagcgtgaacgggatctttgtgatttgttgtctgacttactgtt | LLELQVISLESGDASIH |
| | tcgcccccagatgcagagagagaaggagaaacgagagactgaggagagaaagcgtg | DTVENLIILANNSLSS |
| | aggcccgtg (SEQ ID NO: 637) | NGNVTESGCKECEEL |
| | | EEKNIKEFLQSFVHIV |
| | | QMFINTSRASGGGGS |
| | | GGGGSGGGGSGGG |
| | | GSGGGSLQLLPSWAI |
| | | TLISVNGIFVICCLTYC |
| | | FAPRCRERRRNERLR |
| | | RESVRPV (SEQ ID |
| | | NO: 650) |
| IL-12p40-Lr8- | gctgtgatggcccctagaaccctgctgctgctgctgagcggcgccctggccctgac | AVMAPRTLLLLLSGAL |
| Ar1-E2A-IL-15- | acagacctgggccatatgggaactgaagaaagatgtttatgtcgtagaattggatt | ALTQTWAIWELKKDV |
| Lr1-Ar2 | ggtatccggatgcccctggagaaatggtggtcctcacctgtgacacccctgaagaa | YVVELDWYPDAPGE |
| | gatggtatcacctggaccttggaccagagcagtgaggtcttaggctctggcaaaac | MVVLTCDTPEEDGIT |
| | cctgaccatccaagtcaaagagtttggagatgctggccagtacacctgtcacaaag | WTLDQSSEVLGSGKT |
| | gaggcgaggttctaagccattcgctcctgctgcttcacaaaaaggaagatggaatt | LTIQVKEFGDAGQYT |
| | tggtccactgatattttaaaggaccagaaagaacccaaaaataagacctttctaag | CHKGGEVLSHSLLLLH |
| | atgcgaggccaagaattattctggacgtttcacctgctggtggctgacgacaatca | KKEDGIWSTDILKDQ |
| | gtactgatttgacattcagtgtcaaaagcagcagaggctcttctgacccccaaggg | KEPKNKTFLRCEAKNY |
| | gtgacgtgcggagctgctacactctctgcagagagagtcagaggggacaacaagg | SGRFTCWWLTTISTD |
| | agtatgagtactcagtggagtgccaggaggacagtgcctgcccagctgctgagga | LTFSVKSSRGSSDPQG |
| | gagtctgcccattgaggtcatggtggatgccgttcacaagctcaagtatgaaaact | VTCGAATLSAERVRG |
| | acaccagcagcttcttcatcagggacatcatcaaacctgacccacccaagaacttg | DNKEYEYSVECQEDS |
| | cagctgaagccattaaagaattctcggcaggtggaggtcagctgggagtaccctg | ACPAAEESLPIEVMV |
| | acacctggagtactccacattcctacttctccctgacattctgcgttcaggtccagg | DAVHKLKYENYTSSFF |
| | gcaagagcaagagagaaaagaaagatagagtcttcacggacaagacctcagccac | IRDIIKPDPPKNLQLKP |
| | ggtcatctgccgcaaaaatgccagcattagcgtgcgggcccaggaccgctactata | LKNSRQVEVSWEYPD |
| | gctcatcttggagcgaatgggcatctgtgccctgcagttctggaggaggaggatct | TWSTPHSYFSLTFCV |
| | ggcggaggaggaagtggaggaggaggctctggaggaggcggatctggaggagg | QVQGKSKREKKDRVF |
| | gggatctggaggcggaggatctggagggggaggatctgggggaggaggaagcg | TDKTSATVICRKNASI |
| | gaggaggagggtctctgcagatctacatctgggctcctctggctggaacatgtggc | SVRAQDRYYSSSWSE |
| | gtgctgctgctgagcctggtgatcacaggctctgggcagtgtaccaattatgctctg | WASVPCSSGGGGSG |
| | ctgaagctggctggggacgtggaaagcaaccccggacctggaacctctctgctgtg | GGGSGGGGSGGGGS |
| | ctggatggccctgtgcctgctgggagctgatcacgctgatgccggaatccacgtgtt | GGGGSGGGGSGGG |
| | cattctgggctgcttctctgccggcctgccaaaaactgaggctaactgggtgaatgt | GSGGGGSGGGGSLQI |
| | gattagtgacctgaaaaagatcgaggacctgatccagagtatgcatatcgacgcc | YIWAPLAGTCGVLLLS |
| | accctgtacaccgaatccgacgtgcacccctcctgcaaagtgaccgctatgaagtg | LVITGSGQCTNYALLK |
| | tttcctgctggagctgcaggtgatcagcctggagagcggagacgctagcatccacg | LAGDVESNPGPGTSL |
| | acaccgtggaaaacctgatcatcctggccaacaacagcctgagcagcaacggcaa | LCWMALCLLGADHA |
| | cgtgaccgagtccggatgcaaagagtgcgaggagctggaggagaagaacattaa | DAGIHVFILGCFSAGL |
| | agagttcctgcagagcttcgtgcacatcgtgcagatgttcatcaacaccagcagag | PKTEANWVNVISDLK |
| | ccagcggcggcggaggaagcggcggaggaggcagcggaggaggcggctctgga | KIEDLIQSMHIDATLY |
| | ggcggcggatctggcggcggaagcctgcagctgctgccttcttgggctatcaccct | TESDVHPSCKVTAMK |
| | gatcagcgtgaacggaatttttgtgatctgttgtctgacttactgttttgctccccg | CFLLELQVISLESGDAS |
| | gtgtagggagaggagaagaaacgagagactgagaagagaatcagtgaggcccgtg | IHDTVENLIILANNSLS |
| | (SEQ ID NO: 638) | SNGNVTESGCKECEE |
| | | LEEKNIKEFLQSFVHIV |
| | | QMFINTSRASGGGGS |
| | | GGGGSGGGGSGGG |
| | | GSGGGSLQLLPSWAI |
| | | TLISVNGIFVICCLTYC |
| | | FAPRCRERRRNERLR |
| | | RESVRPV (SEQ ID |
| | | NO: 651) |

TABLE A-4
VIRAL VECTOR CONSTRUCTS WITH THREE CYTOKINES
General structure:
SP1-Cytokine1-Anchoring structure 1-Linker-SP2-Cytokine2-Anchoring structure 2-Linker-SP3-
Cytokine3-Anchoring structure 3

| | | Amino Acid |
|---|---|---|
| Construct | Nucleic Acid Sequence | Sequence |
| SP1-IL-21- | ggaacaagcctgctgtgctggatggctctgtgcctgctgggcgctgaccacgccgac | GTSLLCWMALCLLGA |
| Anchoring | gctcagggccaggacagacacatgatcagaatgagacagctgatcgacatcgtgg | DHADAQGQDRHMI |
| structure 1-T2A- | accagctgaagaactacgtgaacgacctggtgcccgagttcctgcccgctcctgaag | RMRQLIDIVDQLKNY |
| SP2-IL-12p40- | acgtggaaacaaactgtgagtggagcgctttcagctgcttccagaaggcccagctg | VNDLVPEFLPAPEDV |
| Anchoring | aagagcgccaataccggaaacaatgagagaatcatcaacgtgagcatcaagaagc | ETNCEWSAFSCFQKA |
| structure 2-P2A- | tgaagagaaagccccccagcaccaacgccggaagaagacaaaaacatagactga | QLKSANTGNNERIIN |
| IL-15-Anchoring | cctgccccagctgcgatagctacgagaaaaagccccccaaggagttcctggagaga | VSIKKLKRKPPSTNAG |
| structure 3 | ttcaagagcctgctgcagaagatgatccaccagcacctgagcagcagaacccacgg | RRQKHRLTCPSCDSY |
| | ctctgaagatagcagagctagagccgaattcctggagaacggagggacaagcctg | EKKPPKEFLERFKSLL |
| | agcgagaagacagtgctgctgctggtgaccccatttctggccgcagcatggagcctg | QKMIHQHLSSRTHGS |
| | caccccgctggaacgcgtggctccggcgagggcaggggaagtcttctaacatgcgg | EDSRARAEFLENGGT |
| | ggacgtggaggaaaatcccggcccagctagcgctgtgatggcccctagaaccctgc | SLSEKTVLLLVTPFLAA |
| | tgctgctgctgagcggcgccctggccctgacacagacctgggccatatgggaactga | AWSLHPAGTRGSGE |
| | agaaagatgtttatgtcgtagaattggattggtatccggatgcccctggagaaatgg | GRGSLLTCGDVEENP |
| | tggtcctcacctgtgacacccctgaagaagatggtatcacctggaccttggaccaga | GPASAVMAPRTLLLL |
| | gcagtgaggtcttaggctctggcaaaaccctgaccatccaagtcaaagagtttggag | LSGALALTQTWAIWE |
| | atgctggccagtacacctgtcacaaaggaggcgaggttctaagccattcgctcctgc | LKKDVYVVELDWYPD |
| | tgcttcacaaaaaggaagatggaatttggtccactgatattttaaaggaccagaaag | APGEMVVLTCDTPEE |
| | aacccaaaaataagacctttctaagatgcgaggccaagaattattctggacgtttca | DGITWTLDQSSEVLG |
| | cctgctggtggctgacgacaatcagtactgatttgacattcagtgtcaaaagcagca | SGKTLTIQVKEFGDA |
| | gaggctcttctgacccccaaggggtgacgtgcggagctgctacactctctgcagaga | GQYTCHKGGEVLSHS |
| | gagtcagaggggacaacaaggagtatgagtactcagtggagtgccaggaggacag | LLLLHKKEDGIWSTDI |
| | tgcctgcccagctgctgaggagagtctgcccattgaggtcatggtggatgccgttcac | LKDQKEPKNKTFLRCE |
| | aagctcaagtatgaaaactacaccagcagcttcttcatcagggacatcatcaaacct | AKNYSGRFTCWWLT |
| | gacccacccaagaacttgcagctgaagccattaaagaattctcggcaggtggaggt | TISTDLTFSVKSSRGSS |
| | cagctgggagtaccctgacacctggagtactccacattcctacttctccctgacattc | DPQGVTCGAATLSAE |
| | tgcgttcaggtccagggcaagagcaagagagaaaagaaagatagagtcttcacgga | RVRGDNKEYEYSVEC |
| | caagacctcagccacggtcatctgccgcaaaaatgccagcattagcgtgcgggccc | QEDSACPAAEESLPIE |
| | aggaccgctactatagctcatcttggagcgaatgggcatctgtgccctgcagtagag | VMVDAVHKLKYENY |
| | ccgagttcctggagaacggaggaacaagcctgagcgagaaaacagtgctgctgctg | TSSFFIRDIIKPDPPKN |
| | gtgaccccattcctggcagccgcctggagcctgcaccccgctggaatcgatggcagc | LQLKPLKNSRQVEVS |
| | ggcgccacaaacttctctctgctaaagcaagcaggtgatgttgaagaaaaccccgg | WEYPDTWSTPHSYFS |
| | gcctctcgagggcaccagcctgctgtgctggatggcactgtgcctgctgggagcaga | LTFCVQVQGKSKREK |
| | ccacgccgatgccggcatccacgtgttcatcctgggctgcttcagcgccggacttcct | KDRVFTDKTSATVICR |
| | aaaacagaggccaactgggtgaacgtgattagcgacctgaagaagatcgaggacc | KNASISVRAQDRYYSS |
| | tgatccagagcatgcacatcgacgccaccctgtacaccgagagcgatgtgcatccta | SWSEWASVPCSRAEF |
| | gctgcaaggtgaccgccatgaagtgcttcctgctggagctgcaggtgatcagcctgg | LENGGTSLSEKTVLLL |
| | agagcggagatgccagcattcacgacacagtggaaaatctgatcatcctggccaac | VTPFLAAAWSLHPAG |
| | aacagcctgagcagcaacggcaatgtgaccgagagcggctgtaaggagtgcgagg | IDGSGATNFSLLKQA |
| | aactggaggagaagaacatcaaggagttcctgcagagcttcgtgcacatcgtgcag | GDVEENPGPLEGTSL |
| | atgttcatcaacaccagcagagccagggccgagttccttgaaaatggtgggacatcc | LCWMALCLLGADHA |
| | ttatcagagaaaacagttcttctgctggtgactccatttctggcagcagcctggagcc | DAGIHVFILGCFSAGL |
| | ttcatccc (SEQ ID NO: 652) | PKTEANWVNVISDLK |
| | | KIEDLIQSMHIDATLY |
| | | TESDVHPSCKVTAMK |
| | | CFLLELQVISLESGDAS |
| | | IHDTVENLIILANNSLS |
| | | SNGNVTESGCKECEE |
| | | LEEKNIKEFLQSFVHIV |
| | | QMFINTSRARAEFLE |
| | | NGGTSLSEKTVLLLVT |
| | | PFLAAAWSLHP |
| | | (SEQ ID NO: 655) |
| SP1-IL-12p40- | gctgtgatggcccctagaaccctgctgctgctgctgagcggcgccctggccctgaca | AVMAPRTLLLLLSGAL |
| Anchoring | cagacctgggccatatgggaactgaagaaagatgtttatgtcgtagaattggattgg | ALTQTWAIWELKKDV |
| structure 1-T2A- | tatccggatgcccctggagaaatggtggtcctcacctgtgacacccctgaagaagat | YVVELDWYPDAPGE |
| SP2-IL-21- | ggtatcacctggaccttggaccagagcagtgaggtcttaggctctggcaaaaccctg | MVVLTCDTPEEDGIT |
| Anchoring | accatccaagtcaaagagtttggagatgctggccagtacacctgtcacaaaggagg | WTLDQSSEVLGSGKT |
| structure 2-P2A- | cgaggttctaagccattcgctcctgctgcttcacaaaaaggaagatggaatttggtcc | LTIQVKEFGDAGQYT |
| IL-15-Anchoring | actgatattttaaaggaccagaaagaacccaaaaataagacctttctaagatgcga | CHKGGEVLSHSLLLLH |
| structure 3 | ggccaagaattattctggacgtttcacctgctggtggctgacgacaatcagtactgat | KKEDGIWSTDILKDQ |
| | ttgacattcagtgtcaaaagcagcagaggctcttctgacccccaaggggtgacgtgc | KEPKNKTFLRCEAKNY |
| | ggagctgctacactctctgcagagagagtcagaggggacaacaaggagtatgagt | SGRFTCWWLTTISTD |
| | actcagtggagtgccaggaggacagtgcctgcccagctgctgaggagagtctgccc | LTFSVKSSRGSSDPQG |
| | attgaggtcatggtggatgccgttcacaagctcaagtatgaaaactacaccagcagc | VTCGAATLSAERVRG |
| | ttcttcatcagggacatcatcaaacctgacccacccaagaacttgcagctgaagcca | DNKEYEYSVECQEDS |
| | ttaaagaattctcggcaggtggaggtcagctgggagtaccctgacacctggagtact | ACPAAEESLPIEVMV |
| | ccacattcctacttctccctgacattctgcgttcaggtccagggcaagagcaagaga | DAVHKLKYENYTSSFF |
| | gaaaagaaagatagagtcttcacggacaagacctcagccacggtcatctgccgcaa | IRDIIKPDPPKNLQLKP |
| | aaatgccagcattagcgtgcgggcccaggaccgctactatagctcatcttggagcga | LKNSRQVEVSWEYPD |
| | atgggcatctgtgccctgcagtagagccgagttcctggagaacggaggaacaagcc | TWSTPHSYFSLTFCV |
| | tgagcgagaaaacagtgctgctgctggtgaccccattcctggcagccgcctggagcc | QVQGKSKREKKDRVF |
| | tgcaccccgctggaacgcgtggctccggcgagggcaggggaagtcttctaacatgc | TDKTSATVICRKNASI |
| | ggggacgtggaggaaaatcccggcccagctagcggaacaagcctgctgtgctggat | SVRAQDRYYSSSWSE |
| | ggctctgtgcctgctgggcgctgaccacgccgacgctcagggccaggacagacaca | WASVPCSRAEFLENG |
| | tgatcagaatgagacagctgatcgacatcgtggaccagctgaagaactacgtgaac | GTSLSEKTVLLLVTPFL |
| | gacctggtgcccgagttcctgcccgctcctgaagacgtggaaacaaactgtgagtgg | AAAWSLHPAGTRGS |
| | agcgctttcagctgcttccagaaggcccagctgaagagcgccaataccggaaacaa | GEGRGSLLTCGDVEE |
| | tgagagaatcatcaacgtgagcatcaagaagctgaagagaaagccccccagcacc | NPGPASGTSLLCWM |
| | aacgccggaagaagacaaaaacatagactgacctgccccagctgcgatagctacg | ALCLLGADHADAQG |
| | agaaaaagccccccaaggagttcctggagagattcaagagcctgctgcagaagat | QDRHMIRMRQLIDIV |
| | gatccaccagcacctgagcagcagaacccacggctctgaagatagcagagctaga | DQLKNYVNDLVPEFL |
| | gccgaattcctggagaacggagggacaagcctgagcgagaagacagtgctgctgct | PAPEDVETNCEWSAF |
| | ggtgaccccatttctggccgcagcatggagcctgcaccccgctggaatcgatggcag | SCFQKAQLKSANTGN |
| | cggcgccacaaacttctctctgctaaagcaagcaggtgatgttgaagaaaaccccg | NERIINVSIKKLKRKPP |
| | ggcctctcgagggcaccagcctgctgtgctggatggcactgtgcctgctgggagcag | STNAGRRQKHRLTCP |
| | accacgccgatgccggcatccacgtgttcatcctgggctgcttcagcgccggacttcc | SCDSYEKKPPKEFLER |
| | taaaacagaggccaactgggtgaacgtgattagcgacctgaagaagatcgaggac | FKSLLQKMIHQHLSSR |
| | ctgatccagagcatgcacatcgacgccaccctgtacaccgagagcgatgtgcatcct | THGSEDSRARAEFLE |
| | agctgcaaggtgaccgccatgaagtgcttcctgctggagctgcaggtgatcagcctg | NGGTSLSEKTVLLLVT |
| | gagagcggagatgccagcattcacgacacagtggaaaatctgatcatcctggccaa | PFLAAAWSLHPAGID |
| | caacagcctgagcagcaacggcaatgtgaccgagagcggctgtaaggagtgcgag | GSGATNFSLLKQAGD |
| | gaactggaggagaagaacatcaaggagttcctgcagagcttcgtgcacatcgtgca | VEENPGPLEGTSLLC |
| | gatgttcatcaacaccagcagagccagggccgagttccttgaaaatggtgggacat | WMALCLLGADHADA |
| | ccttatcagagaaaacagttcttctgctggtgactccatttctggcagcagcctggag | GIHVFILGCFSAGLPK |
| | ccttcatccc (SEQ ID NO: 653) | TEANWVNVISDLKKIE |
| | | DLIQSMHIDATLYTES |
| | | DVHPSCKVTAMKCFL |
| | | LELQVISLESGDASIH |
| | | DTVENLIILANNSLSS |
| | | NGNVTESGCKECEEL |
| | | EEKNIKEFLQSFVHIV |
| | | QMFINTSRARAEFLE |
| | | NGGTSLSEKTVLLLVT |
| | | PFLAAAWSLHP |
| | | (SEQ ID NO: 656) |
| SP1-IL-15- | ggcaccagcctgctgtgctggatggcactgtgcctgctgggagcagaccacgccgat | GTSLLCWMALCLLGA |
| Anchoring | gccggcatccacgtgttcatcctgggctgcttcagcgccggacttcctaaaacagag | DHADAGIHVFILGCFS |
| structure 1-T2A- | gccaactgggtgaacgtgattagcgacctgaagaagatcgaggacctgatccagag | AGLPKTEANWVNVIS |
| SP2-IL-21- | catgcacatcgacgccaccctgtacaccgagagcgatgtgcatcctagctgcaaggt | DLKKIEDLIQSMHIDA |
| Anchoring | gaccgccatgaagtgcttcctgctggagctgcaggtgatcagcctggagagcggag | TLYTESDVHPSCKVTA |
| structure 2-P2A- | atgccagcattcacgacacagtggaaaatctgatcatcctggccaacaacagcctg | MKCFLLELQVISLESG |
| IL-12p40- | agcagcaacggcaatgtgaccgagagcggctgtaaggagtgcgaggaactggagg | DASIHDTVENLIILAN |
| Anchoring | agaagaacatcaaggagttcctgcagagcttcgtgcacatcgtgcagatgttcatca | NSLSSNGNVTESGCK |
| structure 3 | acaccagcagagccagggccgagttccttgaaaatggtgggacatccttatcagag | ECEELEEKNIKEFLQSF |
| | aaaacagttcttctgctggtgactccatttctggcagcagcctggagccttcatcccg | VHIVQMFINTSRARA |
| | ctggaacgcgtggctccggcgagggcaggggaagtcttctaacatgcggggacgtgg | EFLENGGTSLSEKTVL |
| | aggaaaatcccggcccagctagcggaacaagcctgctgtgctggatggctctgtgcc | LLVTPFLAAAWSLHP |
| | tgctgggcgctgaccacgccgacgctcagggccaggacagacacatgatcagaatg | AGTRGSGEGRGSLLT |
| | agacagctgatcgacatcgtggaccagctgaagaactacgtgaacgacctggtgcc | CGDVEENPGPASGTS |
| | cgagttcctgcccgctcctgaagacgtggaaacaaactgtgagtggagcgctttcag | LLCWMALCLLGADH |
| | ctgcttccagaaggcccagctgaagagcgccaataccggaaacaatgagagaatc | ADAQGQDRHMIRM |
| | atcaacgtgagcatcaagaagctgaagagaaagccccccagcaccaacgccggaa | RQLIDIVDQLKNYVN |
| | gaagacaaaaacatagactgacctgccccagctgcgatagctacgagaaaaagcc | DLVPEFLPAPEDVETN |
| | ccccaaggagttcctggagagattcaagagcctgctgcagaagatgatccaccagc | CEWSAFSCFQKAQLK |
| | acctgagcagcagaacccacggctctgaagatagcagagctagagccgaattcctg | SANTGNNERIINVSIK |
| | gagaacggagggacaagcctgagcgagaagacagtgctgctgctggtgaccccatt | KLKRKPPSTNAGRRQ |
| | tctggccgcagcatggagcctgcaccccgctggaatcgatggcagcggcgccacaa | KHRLTCPSCDSYEKKP |
| | acttctctctgctaaagcaagcaggtgatgttgaagaaaaccccgggcctctcgagg | PKEFLERFKSLLQKMI |
| | ctgtgatggcccctagaaccctgctgctgctgctgagcggcgccctggccctgacac | HQHLSSRTHGSEDSR |
| | agacctgggccatatgggaactgaagaaagatgtttatgtcgtagaattggattggt | ARAEFLENGGTSLSEK |
| | atccggatgcccctggagaaatggtggtcctcacctgtgacacccctgaagaagatg | TVLLLVTPFLAAAWSL |
| | gtatcacctggaccttggaccagagcagtgaggtcttaggctctggcaaaaccctga | HPAGIDGSGATNFSL |
| | ccatccaagtcaaagagtttggagatgctggccagtacacctgtcacaaaggaggc | LKQAGDVEENPGPLE |
| | gaggttctaagccattcgctcctgctgcttcacaaaaaggaagatggaatttggtcca | AVMAPRTLLLLLSGAL |
| | ctgatattttaaaggaccagaaagaacccaaaaataagacctttctaagatgcgag | ALTQTWAIWELKKDV |
| | gccaagaattattctggacgtttcacctgctggtggctgacgacaatcagtactgatt | YVVELDWYPDAPGE |
| | tgacattcagtgtcaaaagcagcagaggctcttctgacccccaaggggtgacgtgcg | MVVLTCDTPEEDGIT |
| | gagctgctacactctctgcagagagagtcagaggggacaacaaggagtatgagtac | WTLDQSSEVLGSGKT |
| | tcagtggagtgccaggaggacagtgcctgcccagctgctgaggagagtctgcccatt | LTIQVKEFGDAGQYT |
| | gaggtcatggtggatgccgttcacaagctcaagtatgaaaactacaccagcagcttc | CHKGGEVLSHSLLLLH |
| | ttcatcagggacatcatcaaacctgacccacccaagaacttgcagctgaagccatta | KKEDGIWSTDILKDQ |
| | aagaattctcggcaggtggaggtcagctgggagtaccctgacacctggagtactcca | KEPKNKTFLRCEAKNY |
| | cattcctacttctccctgacattctgcgttcaggtccagggcaagagcaagagagaa | SGRFTCWWLTTISTD |
| | aagaaagatagagtcttcacggacaagacctcagccacggtcatctgccgcaaaaa | LTFSVKSSRGSSDPQG |
| | tgccagcattagcgtgcgggcccaggaccgctactatagctcatcttggagcgaatg | VTCGAATLSAERVRG |
| | ggcatctgtgccctgcagtagagccgagttcctggagaacggaggaacaagcctga | DNKEYEYSVECQEDS |
| | gcgagaaaacagtgctgctgctggtgaccccattcctggcagccgcctggagcctgc | ACPAAEESLPIEVMV |
| | acccc (SEQ ID NO: 654) | DAVHKLKYENYTSSFF |
| | | IRDIIKPDPPKNLQLKP |
| | | LKNSRQVEVSWEYPD |
| | | TWSTPHSYFSLTFCV |
| | | QVQGKSKREKKDRVF |
| | | TDKTSATVICRKNASI |
| | | SVRAQDRYYSSSWSE |
| | | WASVPCSRAEFLENG |
| | | GTSLSEKTVLLLVTPFL |
| | | AAAWSLHP (SEQ ID |
| | | NO: 657) |

TABLE A-5
VIRAL VECTOR CONSTRUCTS WITH CAR AND ONE CYTOKINE
General structure 1: SP-Cytokine-Anchoring structure-Linker-CAR
General structure 2: CAR-Linker-SP-Cytokine-Anchoring structure

| Construct | Nucleic Acid Sequence | Amino Acid Sequence |
|---|---|---|
| SP-IL-15- | ggcaccagcctgctgtgctggatggcactgtgcctgctgggagcagaccacgccgatgcc | GTSLLCWMALCLLGAD |
| Anchoring | ggcatccacgtgttcatcctgggctgcttcagcgccggacttcctaaaacagaggccaac | HADAGIHVFILGCFSAG |
| structure- | tgggtgaacgtgattagcgacctgaagaagatcgaggacctgatccagagcatgcacat | LPKTEANWVNVISDLKK |
| P2A-CAR19 | cgacgccaccctgtacaccgagagcgatgtgcatcctagctgcaaggtgaccgccatga | IEDLIQSMHIDATLYTES |
| | agtgcttcctgctggagctgcaggtgatcagcctggagagcggagatgccagcattcacg | DVHPSCKVTAMKCFLLE |
| | acacagtggaaaatctgatcatcctggccaacaacagcctgagcagcaacggcaatgtg | LQVISLESGDASIHDTVE |
| | accgagagcggctgtaaggagtgcgaggaactggaggagaagaacatcaaggagttc | NLIILANNSLSSNGNVTE |
| | ctgcagagcttcgtgcacatcgtgcagatgttcatcaacaccagcagagccgaattcctt | SGCKECEELEEKNIKEFL |
| | gaaaatggtgggacatccttatcagagaaaacagttcttctgctggtgactccatttctgg | QSFVHIVQMFINTSRAE |
| | cagcagcctggagccttcatcccggctccggcgccacgaacttctctctgttaaagcaag | FLENGGTSLSEKTVLLLV |
| | caggagacgtggaagaaaaccccggtcccgccttaccagtgaccgccttgctcctgccg | TPFLAAAWSLHPGSGA |
| | ctggccttgctgctccacgccgccaggccggacatccagatgacacagactacatcctcc | TNFSLLKQAGDVEENP |
| | ctgtctgcctctctgggagacagagtcaccatcagttgcagggcaagtcaggacattagt | GPALPVTALLLPLALLLH |
| | aaatatttaaattggtatcagcagaaaccagatggaactgttaaactcctgatctaccat | AARPDIQMTQTTSSLSA |
| | acatcaagattacactcaggagtcccatcaaggttcagtggcagtgggtctggaacagat | SLGDRVTISCRASQDISK |
| | tattctctcaccattagcaacctggagcaagaagatattgccacttacttttgccaacagg | YLNWYQQKPDGTVKLLI |
| | gtaatacgcttccgtacacgttcggaggggggaccaagctggagatcacaggtggcggt | YHTSRLHSGVPSRFSGS |
| | ggctcgggcggtggtgggtcgggtggcggcggatctgaggtgaaactgcaggagtcagg | GSGTDYSLTISNLEQEDI |
| | acctggcctggtggcgccctcacagagcctgtccgtcacatgcactgtctcaggggtctca | ATYFCQQGNTLPYTFG |
| | ttacccgactatggtgtaagctggattcgccagcctccacgaaagggtctggagtggctg | GGTKLEITGGGGSGGG |
| | ggagtaatatggggtagtgaaaccacatactataattcagctctcaaatccagactgacc | GSGGGGSEVKLQESGP |
| | atcatcaaggacaactccaagagccaagttttcttaaaaatgaacagtctgcaaactgat | GLVAPSQSLSVTCTVSG |
| | gacacagccatttactactgtgccaaacattattactacggtggtagctatgctatggact | VSLPDYGVSWIRQPPRK |
| | actggggccaaggaacctcagtcaccgtctcctcaaccacgacgccagcgccgcgacca | GLEWLGVIWGSETTYY |
| | ccaacaccggcgcccaccatcgcgtcgcagcccctgtccctgcgcccagaggcgtgccg | NSALKSRLTIIKDNSKSQ |
| | gccagcggggggggcgcagtgcacacgagggggctggacttcgcctgtgatatctaca | VFLKMNSLQTDDTAIYY |
| | tctgggcgcccttggccgggacttgtggggtccttctcctgtcactggttatcaccctttac | CAKHYYYGGSYAMDY |
| | tgcaaacggggcagaaagaaactcctgtatatattcaaacaaccatttatgagaccagta | WGQGTSVTVSSTTTPA |
| | caaactactcaagaggaagatggctgtagctgccgatttccagaagaagaagaaggag | PRPPTPAPTIASQPLSLR |
| | gatgtgaactgagagtgaagttcagcaggagcgcagacgcccccgcgtaccagcaggg | PEACRPAAGGAVHTRG |
| | ccagaaccagctctataacgagctcaatctaggacgaagagaggagtacgatgttttgg | LDFACDIYIWAPLAGTC |
| | acaagagacgtggccgggaccctgagatggggggaaagccgagaaggaagaaccctc | GVLLLSLVITLYCKRGRK |
| | aggaaggcctgtacaatgaactgcagaaagataagatggcggaggcctacagtgagat | KLLYIFKQPFMRPVQTT |
| | tgggatgaaaggcgagcgccggaggggcaaggggcacgatggcctttaccagggtctc | QEEDGCSCRFPEEEEGG |
| | agtacagccaccaaggacacctacgacgcccttcacatgcaggccctgccccctcgc | CELRVKFSRSADAPAYQ |
| | (SEQ ID NO: 658) | QGQNQLYNELNLGRRE |
| | | EYDVLDKRRGRDPEMG |
| | | GKPRRKNPQEGLYNEL |
| | | QKDKMAEAYSEIGMKG |
| | | ERRRGKGHDGLYQGLS |
| | | TATKDTYDALHMQALP |
| | | PR (SEQ ID NO: 667) |
| SP-IL- | gctgtgatggcccctagaaccctgctgctgctgctgagcggcgccctggccctgacacag | AVMAPRTLLLLLSGALA |
| 12p40- | acctgggccatatgggaactgaagaaagatgtttatgtcgtagaattggattggtatccg | LTQTWAIWELKKDVYV |
| Anchoring | gatgcccctggagaaatggtggtcctcacctgtgacacccctgaagaagatggtatcacc | VELDWYPDAPGEMVVL |
| structure- | tggaccttggaccagagcagtgaggtcttaggctctggcaaaaccctgaccatccaagtc | TCDTPEEDGITWTLDQS |
| P2A-CAR19 | aaagagtttggagatgctggccagtacacctgtcacaaaggaggcgaggttctaagcca | SEVLGSGKTLTIQVKEFG |
| | ttcgctcctgctgcttcacaaaaaggaagatggaatttggtccactgatattttaaaggac | DAGQYTCHKGGEVLSH |
| | cagaaagaacccaaaaataagacctttctaagatgcgaggccaagaattattctggacg | SLLLLHKKEDGIWSTDIL |
| | tttcacctgctggtggctgacgacaatcagtactgatttgacattcagtgtcaaaagcagc | KDQKEPKNKTFLRCEAK |
| | agaggctcttctgacccccaaggggtgacgtgcggagctgctacactctctgcagagag | NYSGRFTCWWLTTISTD |
| | agtcagaggggacaacaaggagtatgagtactcagtggagtgccaggaggacagtgcc | LTFSVKSSRGSSDPQGV |
| | tgcccagctgctgaggagagtctgcccattgaggtcatggtggatgccgttcacaagctc | TCGAATLSAERVRGDN |
| | aagtatgaaaactacaccagcagcttcttcatcagggacatcatcaaacctgacccaccc | KEYEYSVECQEDSACPA |
| | aagaacttgcagctgaagccattaaagaattctcggcaggtggaggtcagctgggagta | AEESLPIEVMVDAVHKL |
| | ccctgacacctggagtactccacattcctacttctccctgacattctgcgttcaggtccagg | KYENYTSSFFIRDIIKPDP |
| | gcaagagcaagagagaaaagaaagatagagtcttcacggacaagacctcagccacgg | PKNLQLKPLKNSRQVEV |
| | tcatctgccgcaaaaatgccagcattagcgtgcgggcccaggaccgctactatagctcat | SWEYPDTWSTPHSYFSL |
| | cttggagcgaatgggcatctgtgccctgcagtagagccgagttcctggagaacggagga | TFCVQVQGKSKREKKD |
| | acaagcctgagcgagaaaacagtgctgctgctggtgaccccattcctggcagccgcctg | RVFTDKTSATVICRKNA |
| | gagcctgcaccccggaagtggagccacgaacttctctctgttaaagcaagcaggagacg | SISVRAQDRYYSSSWSE |
| | tggaagaaaaccccggtccctctcgctccgtggccttagctgtgctcgcgctactctctctt | WASVPCSRAEFLENGG |
| | tctggcctggaggctgacatccagatgacacagactacatcctccctgtctgcctctctgg | TSLSEKTVLLLVTPFLAA |
| | gagacagagtcaccatcagttgcagggcaagtcaggacattagtaaatatttaaattggt | AWSLHPGSGATNFSLLK |
| | atcagcagaaaccagatggaactgttaaactcctgatctaccatacatcaagattacact | QAGDVEENPGPSRSVA |
| | caggagtcccatcaaggttcagtggcagtgggtctggaacagattattctctcaccattag | LAVLALLSLSGLEADIQ |
| | caacctggagcaagaagatattgccacttacttttgccaacagggtaatacgcttccgta | MTQTTSSLSASLGDRVT |
| | cacgttcggaggggggaccaagctggagatcacaggtggcggtggctcgggcggtggt | ISCRASQDISKYLNWYQ |
| | gggtcgggtggcggcggatctgaggtgaaactgcaggagtcaggacctggcctggtggc | QKPDGTVKLLIYHTSRL |
| | gccctcacagagcctgtccgtcacatgcactgtctcaggggtctcattacccgactatggt | HSGVPSRFSGSGSGTDY |
| | gtaagctggattcgccagcctccacgaaagggtctggagtggctgggagtaatatgggg | SLTISNLEQEDIATYFCQ |
| | tagtgaaaccacatactataattcagctctcaaatccagactgaccatcatcaaggacaa | QGNTLPYTFGGGTKLEI |
| | ctccaagagccaagttttcttaaaaatgaacagtctgcaaactgatgacacagccattta | TGGGGSGGGGSGGGG |
| | ctactgtgccaaacattattactacggtggtagctatgctatggactactggggccaagg | SEVKLQESGPGLVAPSQ |
| | aacctcagtcaccgtctcctcaaccacgacgccagcgccgcgaccaccaacaccggcgc | SLSVTCTVSGVSLPDYG |
| | ccaccatcgcgtcgcagcccctgtccctgcgcccagaggcgtgccggccagcggcgggg | VSWIRQPPRKGLEWLG |
| | ggcgcagtgcacacgagggggctggacttcgcctgtgatatctacatctgggcgcccttg | VIWGSETTYYNSALKSR |
| | gccgggacttgtggggtccttctcctgtcactggttatcaccctttactgcaaacggggca | LTIIKDNSKSQVFLKMNS |
| | gaaagaaactcctgtatatattcaaacaaccatttatgagaccagtacaaactactcaag | LQTDDTAIYYCAKHYYY |
| | aggaagatggctgtagctgccgatttccagaagaagaagaaggaggatgtgaactgag | GGSYAMDYWGQGTSV |
| | agtgaagttcagcaggagcgcagacgcccccgcgtaccagcagggccagaaccagctc | TVSSTTTPAPRPPTPAPT |
| | tataacgagctcaatctaggacgaagagaggagtacgatgttttggacaagagacgtgg | IASQPLSLRPEACRPAA |
| | ccgggaccctgagatggggggaaagccgagaaggaagaaccctcaggaaggcctgta | GGAVHTRGLDFACDIYI |
| | caatgaactgcagaaagataagatggcggaggcctacagtgagattgggatgaaaggc | WAPLAGTCGVLLLSLVI |
| | gagcgccggaggggcaaggggcacgatggcctttaccagggtctcagtacagccacca | TLYCKRGRKKLLYIFKQP |
| | aggacacctacgacgcccttcacatgcaggccctgccccctcgc (SEQ ID NO: | FMRPVQTTQEEDGCSC |
| | 659) | RFPEEEEGGCELRVKFS |
| | | RSADAPAYQQGQNQLY |
| | | NELNLGRREEYDVLDKR |
| | | RGRDPEMGGKPRRKN |
| | | PQEGLYNELQKDKMAE |
| | | AYSEIGMKGERRRGKG |
| | | HDGLYQGLSTATKDTYD |
| | | ALHMQALPPR (SEQ |
| | | ID NO: 668) |
| SP-IL-21- | ggcaccagcctgctgtgctggatggcactgtgcctgctgggagcagaccacgccgatgcc | GTSLLCWMALCLLGAD |
| Anchoring | cagggccaggacagacacatgatcagaatgagacagctgatcgacatcgtggaccagc | HADAQGQDRHMIRMR |
| structure- | tgaagaactacgtgaacgacctggtgcccgagttcctgcccgctcctgaagacgtggaa | QLIDIVDQLKNYVNDLV |
| P2A-CAR19 | acaaactgtgagtggagcgctttcagctgcttccagaaggcccagctgaagagcgccaa | PEFLPAPEDVETNCEWS |
| | taccggaaacaatgagagaatcatcaacgtgagcatcaagaagctgaagagaaagccc | AFSCFQKAQLKSANTG |
| | cccagcaccaacgccggaagaagacaaaaacatagactgacctgccccagctgcgata | NNERIINVSIKKLKRKPP |
| | gctacgagaaaaagccccccaaggagttcctggagagattcaagagcctgctgcagaa | STNAGRRQKHRLTCPSC |
| | gatgatccaccagcacctgagcagcagaacccacggctctgaagatagcagagctgaa | DSYEKKPPKEFLERFKSL |
| | ttccttgaaaatggtgggacatccttatcagagaaaacagttcttctgctggtgactccatt | LQKMIHQHLSSRTHGSE |
| | tctggcagcagcctggagccttcatcccggatctggagcaacaaacttctcactactcaa | DSRAEFLENGGTSLSEK |
| | acaagcaggtgacgtggaggagaatccggggcccgccttaccagtgaccgccttgctcc | TVLLLVTPFLAAAWSLH |
| | tgccgctggccttgctgctccacgccgccaggccggacatccagatgacacagactacat | PGSGATNFSLLKQAGD |
| | cctccctgtctgcctctctgggagacagagtcaccatcagttgcagggcaagtcaggaca | VEENPGPALPVTALLLPL |
| | ttagtaaatatttaaattggtatcagcagaaaccagatggaactgttaaactcctgatcta | ALLLHAARPDIQMTOTT |
| | ccatacatcaagattacactcaggagtcccatcaaggttcagtggcagtgggtctggaac | SSLSASLGDRVTISCRAS |
| | agattattctctcaccattagcaacctggagcaagaagatattgccacttacttttgccaa | QDISKYLNWYQQKPDG |
| | cagggtaatacgcttccgtacacgttcggaggggggaccaagctggagatcacaggtgg | TVKLLIYHTSRLHSGVPS |
| | cggtggctcgggcggtggtgggtcgggtggcggcggatctgaggtgaaactgcaggagt | RFSGSGSGTDYSLTISNL |
| | caggacctggcctggtggcgccctcacagagcctgtccgtcacatgcactgtctcagggg | EQEDIATYFCQQGNTLP |
| | tctcattacccgactatggtgtaagctggattcgccagcctccacgaaagggtctggagt | YTFGGGTKLEITGGGGS |
| | ggctgggagtaatatggggtagtgaaaccacatactataattcagctctcaaatccagac | GGGGSGGGGSEVKLQE |
| | tgaccatcatcaaggacaactccaagagccaagttttcttaaaaatgaacagtctgcaaa | SGPGLVAPSQSLSVTCT |
| | ctgatgacacagccatttactactgtgccaaacattattactacggtggtagctatgctat | VSGVSLPDYGVSWIRQ |
| | ggactactggggccaaggaacctcagtcaccgtctcctcaaccacgacgccagcgccgc | PPRKGLEWLGVIWGSE |
| | gaccaccaacaccggcgcccaccatcgcgtcgcagcccctgtccctgcgcccagaggcg | TTYYNSALKSRLTIIKDNS |
| | tgccggccagcggcggggggcgcagtgcacacgagggggctggacttcgcctgtgatat | KSQVFLKMNSLQTDDT |
| | ctacatctgggcgcccttggccgggacttgtggggtccttctcctgtcactggttatcaccc | AIYYCAKHYYYGGSYAM |
| | tttactgcaaacggggcagaaagaaactcctgtatatattcaaacaaccatttatgagac | DYWGQGTSVTVSSTTT |
| | cagtacaaactactcaagaggaagatggctgtagctgccgatttccagaagaagaaga | PAPRPPTPAPTIASQPLS |
| | aggaggatgtgaactgagagtgaagttcagcaggagcgcagacgcccccgcgtaccag | LRPEACRPAAGGAVHT |
| | cagggccagaaccagctctataacgagctcaatctaggacgaagagaggagtacgatg | RGLDFACDIYIWAPLAG |
| | ttttggacaagagacgtggccgggaccctgagatggggggaaagccgagaaggaaga | TCGVLLLSLVITLYCKRG |
| | accctcaggaaggcctgtacaatgaactgcagaaagataagatggcggaggcctacag | RKKLLYIFKQPFMRPVQ |
| | tgagattgggatgaaaggcgagcgccggaggggcaaggggcacgatggcctttaccag | TTQEEDGCSCRFPEEEE |
| | ggtctcagtacagccaccaaggacacctacgacgcccttcacatgcaggccctgccccct | GGCELRVKFSRSADAPA |
| | cgc (SEQ ID NO: 660) | YQQGQNQLYNELNLGR |
| | | REEYDVLDKRRGRDPE |
| | | MGGKPRRKNPQEGLY |
| | | NELQKDKMAEAYSEIG |
| | | MKGERRRGKGHDGLY |
| | | QGLSTATKDTYDALHM |
| | | QALPPR (SEQ ID NO: |
| | | 669) |
| SP-IL- | gctgtgatggcccctagaaccctgctgctgctgctgagcggcgccctggccctgacacag | AVMAPRTLLLLLSGALA |
| 12p40- | acctgggccatatgggaactgaagaaagatgtttatgtcgtagaattggattggtatccg | LTQTWAIWELKKDVYV |
| Anchoring | gatgcccctggagaaatggtggtcctcacctgtgacacccctgaagaagatggtatcacc | VELDWYPDAPGEMVVL |
| structure- | tggaccttggaccagagcagtgaggtcttaggctctggcaaaaccctgaccatccaagtc | TCDTPEEDGITWTLDQS |
| T2A-CAR19 | aaagagtttggagatgctggccagtacacctgtcacaaaggaggcgaggttctaagcca | SEVLGSGKTLTIQVKEFG |
| | ttcgctcctgctgcttcacaaaaaggaagatggaatttggtccactgatattttaaaggac | DAGQYTCHKGGEVLSH |
| | cagaaagaacccaaaaataagacctttctaagatgcgaggccaagaattattctggacg | SLLLLHKKEDGIWSTDIL |
| | tttcacctgctggtggctgacgacaatcagtactgatttgacattcagtgtcaaaagcagc | KDQKEPKNKTFLRCEAK |
| | agaggctcttctgacccccaaggggtgacgtgcggagctgctacactctctgcagagag | NYSGRFTCWWLTTISTD |
| | agtcagaggggacaacaaggagtatgagtactcagtggagtgccaggaggacagtgcc | LTFSVKSSRGSSDPQGV |
| | tgcccagctgctgaggagagtctgcccattgaggtcatggtggatgccgttcacaagctc | TCGAATLSAERVRGDN |
| | aagtatgaaaactacaccagcagcttcttcatcagggacatcatcaaacctgacccaccc | KEYEYSVECQEDSACPA |
| | aagaacttgcagctgaagccattaaagaattctcggcaggtggaggtcagctgggagta | AEESLPIEVMVDAVHKL |
| | ccctgacacctggagtactccacattcctacttctccctgacattctgcgttcaggtccagg | KYENYTSSFFIRDIIKPDP |
| | gcaagagcaagagagaaaagaaagatagagtcttcacggacaagacctcagccacgg | PKNLQLKPLKNSRQVEV |
| | tcatctgccgcaaaaatgccagcattagcgtgcgggcccaggaccgctactatagctcat | SWEYPDTWSTPHSYFSL |
| | cttggagcgaatgggcatctgtgccctgcagtagagccgagttcctggagaacggagga | TFCVQVQGKSKREKKD |
| | acaagcctgagcgagaaaacagtgctgctgctggtgaccccattcctggcagccgcctg | RVFTDKTSATVICRKNA |
| | gagcctgcaccccggaagtggagagggcaggggaagtctactaacatgcggggacgtg | SISVRAQDRYYSSSWSE |
| | gaggaaaatcccggcccctctcgctccgtggccttagctgtgctcgcgctactctctctttc | WASVPCSRAEFLENGG |
| | tggcctggaggctgacatccagatgacacagactacatcctccctgtctgcctctctggga | TSLSEKTVLLLVTPFLAA |
| | gacagagtcaccatcagttgcagggcaagtcaggacattagtaaatatttaaattggtat | AWSLHPGSGEGRGSLL |
| | cagcagaaaccagatggaactgttaaactcctgatctaccatacatcaagattacactca | TCGDVEENPGPSRSVAL |
| | ggagtcccatcaaggttcagtggcagtgggtctggaacagattattctctcaccattagca | AVLALLSLSGLEADIQM |
| | acctggagcaagaagatattgccacttacttttgccaacagggtaatacgcttccgtaca | TQTTSSLSASLGDRVTIS |
| | cgttcggaggggggaccaagctggagatcacaggtggcggtggctcgggcggtggtgg | CRASQDISKYLNWYQQ |
| | gtcgggtggcggcggatctgaggtgaaactgcaggagtcaggacctggcctggtggcgc | KPDGTVKLLIYHTSRLHS |
| | cctcacagagcctgtccgtcacatgcactgtctcaggggtctcattacccgactatggtgt | GVPSRFSGSGSGTDYSL |
| | aagctggattcgccagcctccacgaaagggtctggagtggctgggagtaatatggggta | TISNLEQEDIATYFCQQ |
| | gtgaaaccacatactataattcagctctcaaatccagactgaccatcatcaaggacaact | GNTLPYTFGGGTKLEIT |
| | ccaagagccaagttttcttaaaaatgaacagtctgcaaactgatgacacagccatttact | GGGGSGGGGSGGGGS |
| | actgtgccaaacattattactacggtggtagctatgctatggactactggggccaaggaa | EVKLQESGPGLVAPSQS |
| | cctcagtcaccgtctcctcaaccacgacgccagcgccgcgaccaccaacaccggcgccc | LSVTCTVSGVSLPDYGV |
| | accatcgcgtcgcagcccctgtccctgcgcccagaggcgtgccggccagcgggggggg | SWIRQPPRKGLEWLGVI |
| | cgcagtgcacacgagggggctggacttcgcctgtgatatctacatctgggcgcccttggc | WGSETTYYNSALKSRLTI |
| | cgggacttgtggggtccttctcctgtcactggttatcaccctttactgcaaacggggcaga | IKDNSKSQVFLKMNSLQ |
| | aagaaactcctgtatatattcaaacaaccatttatgagaccagtacaaactactcaagag | TDDTAIYYCAKHYYYGG |
| | gaagatggctgtagctgccgatttccagaagaagaagaaggaggatgtgaactgagag | SYAMDYWGQGTSVTV |
| | tgaagttcagcaggagcgcagacgcccccgcgtaccagcagggccagaaccagctctat | SSTTTPAPRPPTPAPTIA |
| | aacgagctcaatctaggacgaagagaggagtacgatgttttggacaagagacgtggcc | SQPLSLRPEACRPAAGG |
| | gggaccctgagatggggggaaagccgagaaggaagaaccctcaggaaggcctgtaca | AVHTRGLDFACDIYIWA |
| | atgaactgcagaaagataagatggcggaggcctacagtgagattgggatgaaaggcga | PLAGTCGVLLLSLVITLY |
| | gcgccggaggggcaaggggcacgatggcctttaccagggtctcagtacagccaccaag | CKRGRKKLLYIFKQPFM |
| | gacacctacgacgcccttcacatgcaggccctgccccctcgc (SEQ ID NO: 661) | RPVQTTQEEDGCSCRFP |
| | | EEEEGGCELRVKFSRSA |
| | | DAPAYQQGQNQLYNEL |
| | | NLGRREEYDVLDKRRGR |
| | | DPEMGGKPRRKNPQE |
| | | GLYNELQKDKMAEAYS |
| | | EIGMKGERRRGKGHDG |
| | | LYQGLSTATKDTYDALH |
| | | MQALPPR (SEQ ID |
| | | NO: 670) |
| SP-IL-21- | ggcaccagcctgctgtgctggatggcactgtgcctgctgggagcagaccacgccgatgcc | GTSLLCWMALCLLGAD |
| Anchoring | cagggccaggacagacacatgatcagaatgagacagctgatcgacatcgtggaccagc | HADAQGQDRHMIRMR |
| structure- | tgaagaactacgtgaacgacctggtgcccgagttcctgcccgctcctgaagacgtggaa | QLIDIVDQLKNYVNDLV |
| T2A-CAR19 | acaaactgtgagtggagcgctttcagctgcttccagaaggcccagctgaagagcgccaa | PEFLPAPEDVETNCEWS |
| | taccggaaacaatgagagaatcatcaacgtgagcatcaagaagctgaagagaaagccc | AFSCFQKAQLKSANTG |
| | cccagcaccaacgccggaagaagacaaaaacatagactgacctgccccagctgcgata | NNERIINVSIKKLKRKPP |
| | gctacgagaaaaagccccccaaggagttcctggagagattcaagagcctgctgcagaa | STNAGRRQKHRLTCPSC |
| | gatgatccaccagcacctgagcagcagaacccacggctctgaagatagcagagctgaa | DSYEKKPPKEFLERFKSL |
| | ttccttgaaaatggtgggacatccttatcagagaaaacagttcttctgctggtgactccatt | LQKMIHQHLSSRTHGSE |
| | tctggcagcagcctggagccttcatcccggctccggcgagggcaggggaagtctactaa | DSRAEFLENGGTSLSEK |
| | catgcggggacgtggaggaaaatcccggccccgccttaccagtgaccgccttgctcctgc | TVLLLVTPFLAAAWSLH |
| | cgctggccttgctgctccacgccgccaggccggacatccagatgacacagactacatcct | PGSGEGRGSLLTCGDVE |
| | ccctgtctgcctctctgggagacagagtcaccatcagttgcagggcaagtcaggacatta | ENPGPALPVTALLLPLAL |
| | gtaaatatttaaattggtatcagcagaaaccagatggaactgttaaactcctgatctacc | LLHAARPDIQMTOTTSS |
| | atacatcaagattacactcaggagtcccatcaaggttcagtggcagtgggtctggaacag | LSASLGDRVTISCRASQ |
| | attattctctcaccattagcaacctggagcaagaagatattgccacttacttttgccaaca | DISKYLNWYQQKPDGT |
| | gggtaatacgcttccgtacacgttcggaggggggaccaagctggagatcacaggtggcg | VKLLIYHTSRLHSGVPSR |
| | gtggctcgggcggtggtgggtcgggtggcggcggatctgaggtgaaactgcaggagtca | FSGSGSGTDYSLTISNLE |
| | ggacctggcctggtggcgccctcacagagcctgtccgtcacatgcactgtctcaggggtct | QEDIATYFCQQGNTLPY |
| | cattacccgactatggtgtaagctggattcgccagcctccacgaaagggtctggagtggc | TFGGGTKLEITGGGGSG |
| | tgggagtaatatggggtagtgaaaccacatactataattcagctctcaaatccagactga | GGGSGGGGSEVKLQES |
| | ccatcatcaaggacaactccaagagccaagttttcttaaaaatgaacagtctgcaaactg | GPGLVAPSQSLSVTCTV |
| | atgacacagccatttactactgtgccaaacattattactacggtggtagctatgctatgga | SGVSLPDYGVSWIRQPP |
| | ctactggggccaaggaacctcagtcaccgtctcctcaaccacgacgccagcgccgcgac | RKGLEWLGVIWGSETT |
| | caccaacaccggcgcccaccatcgcgtcgcagcccctgtccctgcgcccagaggcgtgc | YYNSALKSRLTIIKDNSKS |
| | cggccagcggcggggggcgcagtgcacacgagggggctggacttcgcctgtgatatcta | QVFLKMNSLQTDDTAIY |
| | catctgggcgcccttggccgggacttgtggggtccttctcctgtcactggttatcacccttt | YCAKHYYYGGSYAMDY |
| | actgcaaacggggcagaaagaaactcctgtatatattcaaacaaccatttatgagacca | WGQGTSVTVSSTTTPA |
| | gtacaaactactcaagaggaagatggctgtagctgccgatttccagaagaagaagaag | PRPPTPAPTIASQPLSLR |
| | gaggatgtgaactgagagtgaagttcagcaggagcgcagacgcccccgcgtaccagca | PEACRPAAGGAVHTRG |
| | gggccagaaccagctctataacgagctcaatctaggacgaagagaggagtacgatgttt | LDFACDIYIWAPLAGTC |
| | tggacaagagacgtggccgggaccctgagatggggggaaagccgagaaggaagaacc | GVLLLSLVITLYCKRGRK |
| | ctcaggaaggcctgtacaatgaactgcagaaagataagatggcggaggcctacagtga | KLLYIFKQPFMRPVQTT |
| | gattgggatgaaaggcgagcgccggaggggcaaggggcacgatggcctttaccagggt | QEEDGCSCRFPEEEEGG |
| | ctcagtacagccaccaaggacacctacgacgcccttcacatgcaggccctgccccctcgc | CELRVKFSRSADAPAYQ |
| | (SEQ ID NO: 662) | QGQNQLYNELNLGRRE |
| | | EYDVLDKRRGRDPEMG |
| | | GKPRRKNPQEGLYNEL |
| | | QKDKMAEAYSEIGMKG |
| | | ERRRGKGHDGLYQGLS |
| | | TATKDTYDALHMQALP |
| | | PR (SEQ ID NO: 671) |
| CAR19- | gccttaccagtgaccgccttgctcctgccgctggccttgctgctccacgccgccaggccgg | ALPVTALLLPLALLLHAA |
| P2A-SP-IL- | acatccagatgacacagactacatcctccctgtctgcctctctgggagacagagtcacca | RPDIQMTQTTSSLSASL |
| 15- | tcagttgcagggcaagtcaggacattagtaaatatttaaattggtatcagcagaaaccag | GDRVTISCRASQDISKYL |
| Anchoring | atggaactgttaaactcctgatctaccatacatcaagattacactcaggagtcccatcaa | NWYQQKPDGTVKLLIY |
| structure | ggttcagtggcagtgggtctggaacagattattctctcaccattagcaacctggagcaag | HTSRLHSGVPSRFSGSG |
| | aagatattgccacttacttttgccaacagggtaatacgcttccgtacacgttcggaggggg | SGTDYSLTISNLEQEDIA |
| | gaccaagctggagatcacaggtggcggtggctcgggcggtggtgggtcgggtggcggc | TYFCQQGNTLPYTFGG |
| | ggatctgaggtgaaactgcaggagtcaggacctggcctggtggcgccctcacagagcct | GTKLEITGGGGSGGGG |
| | gtccgtcacatgcactgtctcaggggtctcattacccgactatggtgtaagctggattcgc | SGGGGSEVKLQESGPG |
| | cagcctccacgaaagggtctggagtggctgggagtaatatggggtagtgaaaccacata | LVAPSQSLSVTCTVSGV |
| | ctataattcagctctcaaatccagactgaccatcatcaaggacaactccaagagccaagt | SLPDYGVSWIRQPPRKG |
| | tttcttaaaaatgaacagtctgcaaactgatgacacagccatttactactgtgccaaacat | LEWLGVIWGSETTYYNS |
| | tattactacggtggtagctatgctatggactactggggccaaggaacctcagtcaccgtct | ALKSRLTIIKDNSKSQVF |
| | cctcaaccacgacgccagcgccgcgaccaccaacaccggcgcccaccatcgcgtcgca | LKMNSLQTDDTAIYYCA |
| | gcccctgtccctgcgcccagaggcgtgccggccagcggcggggggcgcagtgcacacg | KHYYYGGSYAMDYWG |
| | agggggctggacttcgcctgtgatatctacatctgggcgcccttggccgggacttgtgggg | QGTSVTVSSTTTPAPRP |
| | tccttctcctgtcactggttatcaccctttactgcaaacggggcagaaagaaactcctgtat | PTPAPTIASQPLSLRPEA |
| | atattcaaacaaccatttatgagaccagtacaaactactcaagaggaagatggctgtag | CRPAAGGAVHTRGLDF |
| | ctgccgatttccagaagaagaagaaggaggatgtgaactgagagtgaagttcagcagg | ACDIYIWAPLAGTCGVL |
| | agcgcagacgcccccgcgtaccagcagggccagaaccagctctataacgagctcaatct | LLSLVITLYCKRGRKKLLY |
| | aggacgaagagaggagtacgatgttttggacaagagacgtggccgggaccctgagatg | IFKQPFMRPVQTTQEE |
| | gggggaaagccgagaaggaagaaccctcaggaaggcctgtacaatgaactgcagaaa | DGCSCRFPEEEEGGCEL |
| | gataagatggcggaggcctacagtgagattgggatgaaaggcgagcgccggaggggc | RVKFSRSADAPAYQQG |
| | aaggggcacgatggcctttaccagggtctcagtacagccaccaaggacacctacgacgc | QNQLYNELNLGRREEY |
| | ccttcacatgcaggccctgccccctcgcggctccggcgccacgaacttctctctgttaaag | DVLDKRRGRDPEMGG |
| | caagcaggagacgtggaagaaaaccccggtcccggcaccagcctgctgtgctggatgg | KPRRKNPQEGLYNELQ |
| | cactgtgcctgctgggagcagaccacgccgatgccggcatccacgtgttcatcctgggct | KDKMAEAYSEIGMKGE |
| | gcttcagcgccggacttcctaaaacagaggccaactgggtgaacgtgattagcgacctg | RRRGKGHDGLYQGLST |
| | aagaagatcgaggacctgatccagagcatgcacatcgacgccaccctgtacaccgaga | ATKDTYDALHMQALPP |
| | gcgatgtgcatcctagctgcaaggtgaccgccatgaagtgcttcctgctggagctgcagg | RGSGATNFSLLKQAGD |
| | tgatcagcctggagagcggagatgccagcattcacgacacagtggaaaatctgatcatc | VEENPGPGTSLLCWMA |
| | ctggccaacaacagcctgagcagcaacggcaatgtgaccgagagcggctgtaaggagt | LCLLGADHADAGIHVFIL |
| | gcgaggaactggaggagaagaacatcaaggagttcctgcagagcttcgtgcacatcgtg | GCFSAGLPKTEANWVN |
| | cagatgttcatcaacaccagcagagccgaattccttgaaaatggtgggacatccttatca | VISDLKKIEDLIQSMHID |
| | gagaaaacagttcttctgctggtgactccatttctggcagcagcctggagccttcatccc | ATLYTESDVHPSCKVTA |
| | (SEQ ID NO: 663) | MKCFLLELQVISLESGD |
| | | ASIHDTVENLIILANNSL |
| | | SSNGNVTESGCKECEEL |
| | | EEKNIKEFLQSFVHIVQ |
| | | MFINTSRAEFLENGGTS |
| | | LSEKTVLLLVTPFLAAA |
| | | WSLHP (SEQ ID NO: |
| | | 672) |
| CAR19- | gccttaccagtgaccgccttgctcctgccgctggccttgctgctccacgccgccaggccgg | ALPVTALLLPLALLLHAA |
| P2A-SP-IL- | acatccagatgacacagactacatcctccctgtctgcctctctgggagacagagtcacca | RPDIQMTQTTSSLSASL |
| 12p40- | tcagttgcagggcaagtcaggacattagtaaatatttaaattggtatcagcagaaaccag | GDRVTISCRASQDISKYL |
| Anchoring | atggaactgttaaactcctgatctaccatacatcaagattacactcaggagtcccatcaa | NWYQQKPDGTVKLLIY |
| structure | ggttcagtggcagtgggtctggaacagattattctctcaccattagcaacctggagcaag | HTSRLHSGVPSRFSGSG |
| | aagatattgccacttacttttgccaacagggtaatacgcttccgtacacgttcggaggggg | SGTDYSLTISNLEQEDIA |
| | gaccaagctggagatcacaggtggcggtggctcgggcggtggtgggtcgggtggcggc | TYFCQQGNTLPYTFGG |
| | ggatctgaggtgaaactgcaggagtcaggacctggcctggtggcgccctcacagagcct | GTKLEITGGGGSGGGG |
| | gtccgtcacatgcactgtctcaggggtctcattacccgactatggtgtaagctggattcgc | SGGGGSEVKLQESGPG |
| | cagcctccacgaaagggtctggagtggctgggagtaatatggggtagtgaaaccacata | LVAPSQSLSVTCTVSGV |
| | ctataattcagctctcaaatccagactgaccatcatcaaggacaactccaagagccaagt | SLPDYGVSWIRQPPRKG |
| | tttcttaaaaatgaacagtctgcaaactgatgacacagccatttactactgtgccaaacat | LEWLGVIWGSETTYYNS |
| | tattactacggtggtagctatgctatggactactggggccaaggaacctcagtcaccgtct | ALKSRLTIIKDNSKSQVF |
| | cctcaaccacgacgccagcgccgcgaccaccaacaccggcgcccaccatcgcgtcgca | LKMNSLQTDDTAIYYCA |
| | gcccctgtccctgcgcccagaggcgtgccggccagcggcggggggcgcagtgcacacg | KHYYYGGSYAMDYWG |
| | agggggctggacttcgcctgtgatatctacatctgggcgcccttggccgggacttgtgggg | QGTSVTVSSTTTPAPRP |
| | tccttctcctgtcactggttatcaccctttactgcaaacggggcagaaagaaactcctgtat | PTPAPTIASQPLSLRPEA |
| | atattcaaacaaccatttatgagaccagtacaaactactcaagaggaagatggctgtag | CRPAAGGAVHTRGLDF |
| | ctgccgatttccagaagaagaagaaggaggatgtgaactgagagtgaagttcagcagg | ACDIYIWAPLAGTCGVL |
| | agcgcagacgcccccgcgtaccagcagggccagaaccagctctataacgagctcaatct | LLSLVITLYCKRGRKKLLY |
| | aggacgaagagaggagtacgatgttttggacaagagacgtggccgggaccctgagatg | IFKQPFMRPVQTTQEE |
| | gggggaaagccgagaaggaagaaccctcaggaaggcctgtacaatgaactgcagaaa | DGCSCRFPEEEEGGCEL |
| | gataagatggcggaggcctacagtgagattgggatgaaaggcgagcgccggaggggc | RVKFSRSADAPAYQQG |
| | aaggggcacgatggcctttaccagggtctcagtacagccaccaaggacacctacgacgc | QNQLYNELNLGRREEY |
| | ccttcacatgcaggccctgccccctcgcggatctggagcaacaaacttctcactactcaa | DVLDKRRGRDPEMGG |
| | acaagcaggtgacgtggaggagaatccggggcccgctgtgatggcccctagaaccctgc | KPRRKNPQEGLYNELQ |
| | tgctgctgctgagcggcgccctggccctgacacagacctgggcccaccatcatcatcatc | KDKMAEAYSEIGMKGE |
| | atgaattggactacaaagacgatgacgacaaggaattgatatgggaactgaagaaaga | RRRGKGHDGLYQGLST |
| | tgtttatgtcgtagaattggattggtatccggatgcccctggagaaatggtggtcctcacct | ATKDTYDALHMQALPP |
| | gtgacacccctgaagaagatggtatcacctggaccttggaccagagcagtgaggtctta | RGSGATNFSLLKQAGD |
| | ggctctggcaaaaccctgaccatccaagtcaaagagtttggagatgctggccagtacac | VEENPGPAVMAPRTLLL |
| | ctgtcacaaaggaggcgaggttctaagccattcgctcctgctgcttcacaaaaaggaag | LLSGALALTQTWAHHH |
| | atggaatttggtccactgatattttaaaggaccagaaagaacccaaaaataagacctttc | HHHELDYKDDDDKELI |
| | taagatgcgaggccaagaattattctggacgtttcacctgctggtggctgacgacaatca | WELKKDVYVVELDWYP |
| | gtactgatttgacattcagtgtcaaaagcagcagaggctcttctgacccccaaggggtga | DAPGEMVVLTCDTPEE |
| | cgtgcggagctgctacactctctgcagagagagtcagaggggacaacaaggagtatga | DGITWTLDQSSEVLGSG |
| | gtactcagtggagtgccaggaggacagtgcctgcccagctgctgaggagagtctgccca | KTLTIQVKEFGDAGQYT |
| | ttgaggtcatggtggatgccgttcacaagctcaagtatgaaaactacaccagcagcttctt | CHKGGEVLSHSLLLLHK |
| | catcagggacatcatcaaacctgacccacccaagaacttgcagctgaagccattaaaga | KEDGIWSTDILKDQKEP |
| | attctcggcaggtggaggtcagctgggagtaccctgacacctggagtactccacattcct | KNKTFLRCEAKNYSGRF |
| | acttctccctgacattctgcgttcaggtccagggcaagagcaagagagaaaagaaagat | TCWWLTTISTDLTFSVK |
| | agagtcttcacggacaagacctcagccacggtcatctgccgcaaaaatgccagcattag | SSRGSSDPQGVTCGAA |
| | cgtgcgggcccaggaccgctactatagctcatcttggagcgaatgggcatctgtgccctg | TLSAERVRGDNKEYEYS |
| | cagtgaattccttgaaaatggtgggacatccttatcagagaaaacagttcttctgctggtg | VECQEDSACPAAEESLPI |
| | actccatttctggcagcagcctggagccttcatccc (SEQ ID NO: 664) | EVMVDAVHKLKYENYT |
| | | SSFFIRDIIKPDPPKNLOL |
| | | KPLKNSRQVEVSWEYP |
| | | DTWSTPHSYFSLTFCVQ |
| | | VQGKSKREKKDRVFTD |
| | | KTSATVICRKNASISVRA |
| | | QDRYYSSSWSEWASVP |
| | | CSEFLENGGTSLSEKTVL |
| | | LLVTPFLAAAWSLHP |
| | | (SEQ ID NO: 673) |
| CAR19- | gccttaccagtgaccgccttgctcctgccgctggccttgctgctccacgccgccaggccgg | ALPVTALLLPLALLLHAA |
| T2A-SP-IL- | acatccagatgacacagactacatcctccctgtctgcctctctgggagacagagtcacca | RPDIQMTQTTSSLSASL |
| 12p40- | tcagttgcagggcaagtcaggacattagtaaatatttaaattggtatcagcagaaaccag | GDRVTISCRASQDISKYL |
| Anchoring | atggaactgttaaactcctgatctaccatacatcaagattacactcaggagtcccatcaa | NWYQQKPDGTVKLLIY |
| structure | ggttcagtggcagtgggtctggaacagattattctctcaccattagcaacctggagcaag | HTSRLHSGVPSRFSGSG |
| | aagatattgccacttacttttgccaacagggtaatacgcttccgtacacgttcggaggggg | SGTDYSLTISNLEQEDIA |
| | gaccaagctggagatcacaggtggcggtggctcgggcggtggtgggtcgggtggcggc | TYFCQQGNTLPYTFGG |
| | ggatctgaggtgaaactgcaggagtcaggacctggcctggtggcgccctcacagagcct | GTKLEITGGGGSGGGG |
| | gtccgtcacatgcactgtctcaggggtctcattacccgactatggtgtaagctggattcgc | SGGGGSEVKLQESGPG |
| | cagcctccacgaaagggtctggagtggctgggagtaatatggggtagtgaaaccacata | LVAPSQSLSVTCTVSGV |
| | ctataattcagctctcaaatccagactgaccatcatcaaggacaactccaagagccaagt | SLPDYGVSWIRQPPRKG |
| | tttcttaaaaatgaacagtctgcaaactgatgacacagccatttactactgtgccaaacat | LEWLGVIWGSETTYYNS |
| | tattactacggtggtagctatgctatggactactggggccaaggaacctcagtcaccgtct | ALKSRLTIIKDNSKSQVF |
| | cctcaaccacgacgccagcgccgcgaccaccaacaccggcgcccaccatcgcgtcgca | LKMNSLQTDDTAIYYCA |
| | gcccctgtccctgcgcccagaggcgtgccggccagcggcggggggcgcagtgcacacg | KHYYYGGSYAMDYWG |
| | agggggctggacttcgcctgtgatatctacatctgggcgcccttggccgggacttgtgggg | QGTSVTVSSTTTPAPRP |
| | tccttctcctgtcactggttatcaccctttactgcaaacggggcagaaagaaactcctgtat | PTPAPTIASQPLSLRPEA |
| | atattcaaacaaccatttatgagaccagtacaaactactcaagaggaagatggctgtag | CRPAAGGAVHTRGLDF |
| | ctgccgatttccagaagaagaagaaggaggatgtgaactgagagtgaagttcagcagg | ACDIYIWAPLAGTCGVL |
| | agcgcagacgcccccgcgtaccagcagggccagaaccagctctataacgagctcaatct | LLSLVITLYCKRGRKKLLY |
| | aggacgaagagaggagtacgatgttttggacaagagacgtggccgggaccctgagatg | IFKQPFMRPVQTTQEE |
| | gggggaaagccgagaaggaagaaccctcaggaaggcctgtacaatgaactgcagaaa | DGCSCRFPEEEEGGCEL |
| | gataagatggcggaggcctacagtgagattgggatgaaaggcgagcgccggaggggc | RVKFSRSADAPAYQQG |
| | aaggggcacgatggcctttaccagggtctcagtacagccaccaaggacacctacgacgc | QNQLYNELNLGRREEY |
| | ccttcacatgcaggccctgccccctcgcggctccggcgagggcaggggaagtctactaac | DVLDKRRGRDPEMGG |
| | atgcggggacgtggaggaaaatcccggccccgctgtgatggcccctagaaccctgctgc | KPRRKNPQEGLYNELQ |
| | tgctgctgagcggcgccctggccctgacacagacctgggcccaccatcatcatcatcatg | KDKMAEAYSEIGMKGE |
| | aattggactacaaagacgatgacgacaaggaattgatatgggaactgaagaaagatgt | RRRGKGHDGLYQGLST |
| | ttatgtcgtagaattggattggtatccggatgcccctggagaaatggtggtcctcacctgt | ATKDTYDALHMQALPP |
| | gacacccctgaagaagatggtatcacctggaccttggaccagagcagtgaggtcttagg | RGSGEGRGSLLTCGDVE |
| | ctctggcaaaaccctgaccatccaagtcaaagagtttggagatgctggccagtacacctg | ENPGPAVMAPRTLLLLL |
| | tcacaaaggaggcgaggttctaagccattcgctcctgctgcttcacaaaaaggaagatg | SGALALTQTWAHHHH |
| | gaatttggtccactgatattttaaaggaccagaaagaacccaaaaataagacctttctaa | HHELDYKDDDDKELIW |
| | gatgcgaggccaagaattattctggacgtttcacctgctggtggctgacgacaatcagta | ELKKDVYVVELDWYPD |
| | ctgatttgacattcagtgtcaaaagcagcagaggctcttctgacccccaaggggtgacgt | APGEMVVLTCDTPEED |
| | gcggagctgctacactctctgcagagagagtcagaggggacaacaaggagtatgagta | GITWTLDQSSEVLGSGK |
| | ctcagtggagtgccaggaggacagtgcctgcccagctgctgaggagagtctgcccattg | TLTIQVKEFGDAGQYTC |
| | aggtcatggtggatgccgttcacaagctcaagtatgaaaactacaccagcagcttcttca | HKGGEVLSHSLLLLHKK |
| | tcagggacatcatcaaacctgacccacccaagaacttgcagctgaagccattaaagaat | EDGIWSTDILKDQKEPK |
| | tctcggcaggtggaggtcagctgggagtaccctgacacctggagtactccacattcctac | NKTFLRCEAKNYSGRFT |
| | ttctccctgacattctgcgttcaggtccagggcaagagcaagagagaaaagaaagatag | CWWLTTISTDLTFSVKS |
| | agtcttcacggacaagacctcagccacggtcatctgccgcaaaaatgccagcattagcgt | SRGSSDPQGVTCGAAT |
| | gcgggcccaggaccgctactatagctcatcttggagcgaatgggcatctgtgccctgcag | LSAERVRGDNKEYEYSV |
| | tgaattccttgaaaatggtgggacatccttatcagagaaaacagttcttctgctggtgact | ECQEDSACPAAEESLPIE |
| | ccatttctggcagcagcctggagccttcatccc (SEQ ID NO: 665) | VMVDAVHKLKYENYTS |
| | | SFFIRDIIKPDPPKNLQLK |
| | | PLKNSRQVEVSWEYPD |
| | | TWSTPHSYFSLTFCVQV |
| | | QGKSKREKKDRVFTDKT |
| | | SATVICRKNASISVRAQ |
| | | DRYYSSSWSEWASVPC |
| | | SEFLENGGTSLSEKTVLL |
| | | LVTPFLAAAWSLHP |
| | | (SEQ ID NO: 674) |
| CAR19- | gccttaccagtgaccgccttgctcctgccgctggccttgctgctccacgccgccaggccgg | ALPVTALLLPLALLLHAA |
| T2A-SP-IL- | acatccagatgacacagactacatcctccctgtctgcctctctgggagacagagtcacca | RPDIQMTQTTSSLSASL |
| 21- | tcagttgcagggcaagtcaggacattagtaaatatttaaattggtatcagcagaaaccag | GDRVTISCRASQDISKYL |
| Anchoring | atggaactgttaaactcctgatctaccatacatcaagattacactcaggagtcccatcaa | NWYQQKPDGTVKLLIY |
| structure | ggttcagtggcagtgggtctggaacagattattctctcaccattagcaacctggagcaag | HTSRLHSGVPSRFSGSG |
| | aagatattgccacttacttttgccaacagggtaatacgcttccgtacacgttcggaggggg | SGTDYSLTISNLEQEDIA |
| | gaccaagctggagatcacaggtggcggtggctcgggcggtggtgggtcgggtggcggc | TYFCQQGNTLPYTFGG |
| | ggatctgaggtgaaactgcaggagtcaggacctggcctggtggcgccctcacagagcct | GTKLEITGGGGSGGGG |
| | gtccgtcacatgcactgtctcaggggtctcattacccgactatggtgtaagctggattcgc | SGGGGSEVKLQESGPG |
| | cagcctccacgaaagggtctggagtggctgggagtaatatggggtagtgaaaccacata | LVAPSQSLSVTCTVSGV |
| | ctataattcagctctcaaatccagactgaccatcatcaaggacaactccaagagccaagt | SLPDYGVSWIRQPPRKG |
| | tttcttaaaaatgaacagtctgcaaactgatgacacagccatttactactgtgccaaacat | LEWLGVIWGSETTYYNS |
| | tattactacggtggtagctatgctatggactactggggccaaggaacctcagtcaccgtct | ALKSRLTIIKDNSKSQVF |
| | cctcaaccacgacgccagcgccgcgaccaccaacaccggcgcccaccatcgcgtcgca | LKMNSLQTDDTAIYYCA |
| | gcccctgtccctgcgcccagaggcgtgccggccagcggcggggggcgcagtgcacacg | KHYYYGGSYAMDYWG |
| | agggggctggacttcgcctgtgatatctacatctgggcgcccttggccgggacttgtgggg | QGTSVTVSSTTTPAPRP |
| | tccttctcctgtcactggttatcaccctttactgcaaacggggcagaaagaaactcctgtat | PTPAPTIASQPLSLRPEA |
| | atattcaaacaaccatttatgagaccagtacaaactactcaagaggaagatggctgtag | CRPAAGGAVHTRGLDF |
| | ctgccgatttccagaagaagaagaaggaggatgtgaactgagagtgaagttcagcagg | ACDIYIWAPLAGTCGVL |
| | agcgcagacgcccccgcgtaccagcagggccagaaccagctctataacgagctcaatct | LLSLVITLYCKRGRKKLLY |
| | aggacgaagagaggagtacgatgttttggacaagagacgtggccgggaccctgagatg | IFKQPFMRPVQTTQEE |
| | gggggaaagccgagaaggaagaaccctcaggaaggcctgtacaatgaactgcagaaa | DGCSCRFPEEEEGGCEL |
| | gataagatggcggaggcctacagtgagattgggatgaaaggcgagcgccggaggggc | RVKFSRSADAPAYQQG |
| | aaggggcacgatggcctttaccagggtctcagtacagccaccaaggacacctacgacgc | QNQLYNELNLGRREEY |
| | ccttcacatgcaggccctgccccctcgcggctccggcgagggcaggggaagtctactaac | DVLDKRRGRDPEMGG |
| | atgcggggacgtggaggaaaatcccggccccGGCACCAGCCTGCTGTGCTGG | KPRRKNPQEGLYNELQ |
| | ATGGCACTGTGCCTGCTGGGAGCAGACCACGCCGATGCCCAGGGC | KDKMAEAYSEIGMKGE |
| | CAGGACAGACACATGATCAGAATGAGACAGCTGATCGACATCGTG | RRRGKGHDGLYQGLST |
| | GACCAGCTGAAGAACTACGTGAACGACCTGGTGCCCGAGTTCCTGC | ATKDTYDALHMQALPP |
| | CCGCTCCTGAAGACGTGGAAACAAACTGTGAGTGGAGCGCTTTCA | RGSGEGRGSLLTCGDVE |
| | GCTGCTTCCAGAAGGCCCAGCTGAAGAGCGCCAATACCGGAAACA | ENPGPGTSLLCWMALC |
| | ATGAGAGAATCATCAACGTGAGCATCAAGAAGCTGAAGAGAAAGC | LLGADHADAQGQDRH |
| | CCCCCAGCACCAACGCCGGAAGAAGACAAAAACATAGACTGACCT | MIRMRQLIDIVDQLKNY |
| | GCCCCAGCTGCGATAGCTACGAGAAAAAGCCCCCCAAGGAGTTCCT | VNDLVPEFLPAPEDVET |
| | GGAGAGATTCAAGAGCCTGCTGCAGAAGATGATCCACCAGCACCT | NCEWSAFSCFQKAQLK |
| | GAGCAGCAGAACCCACGGCTCTGAAGATAGCAGAGCTgaattccttga | SANTGNNERIINVSIKKL |
| | aaatggtgggacatccttatcagagaaaacagttcttctgctggtgactccatttctggca | KRKPPSTNAGRRQKHR |
| | gcagcctggagccttcatccc (SEQ ID NO: 666) | LTCPSCDSYEKKPPKEFL |
| | | ERFKSLLQKMIHQHLSS |
| | | RTHGSEDSRAEFLENGG |
| | | TSLSEKTVLLLVTPFLAA |
| | | AWSLHP (SEQ ID NO: |
| | | 675) |

TABLE A-6
VIRAL VECTOR CONSTRUCTS WITH CAR AND TWO CYTOKINES
General structure 1: CAR-Linker-SP1-Cytokine1-Anchoring
structure 1-Linker-SP2-Cytokine2-Anchoring structure 2
General structure 2: SP1-Cytokine1-Anchoring structure 1-
Linker-SP2-Cytokine2-Anchoring structure 2-Linker-CAR
General structure 3: SP1-Cytokine1-Anchoring structure 1-
Linker-CAR19-Linker-SP2-Cytokine2-Anchoring structure 2

| | | Amino Acid |
|---|---|---|
| Construct | Nucleic Acid Sequence | Sequence |
| CAR19-T2A- | tctcgctccgtggccttagctgtgctcgcgctactctctcttt | SRSVALAVLALLSLSGLE |
| SP1-IL-15- | ctggcctggaggctgacatccagatgacacagactacatcctc | ADIQMTQTTSSLSASLG |
| Anchoring | cctgtctgcctctctgggagacagagtcaccatcagttgcagg | DRVTISCRASQDISKYLN |
| structure 1- | gcaagtcaggacattagtaaatatttaaattggtatcagcaga | WYQQKPDGTVKLLIYH |
| P2A-SP2-IL- | aaccagatggaactgttaaactcctgatctaccatacatcaag | TSRLHSGVPSRFSGSGS |
| 12p40- | attacactcaggagtcccatcaaggttcagtggcagtgggtct | GTDYSLTISNLEQEDIAT |
| Anchoring | ggaacagattattctctcaccattagcaacctggagcaagaag | YFCQQGNTLPYTFGGG |
| structure 2 | atattgccacttacttttgccaacagggtaatacgcttccgta | TKLEITGGGGSGGGGS |
| | cacgttcggaggggggaccaagctggagatcacaggtggcggt | GGGGSEVKLQESGPGL |
| | ggctcgggcggtggtgggtcgggtggcggcggatctgaggtga | VAPSQSLSVTCTVSGVS |
| | aactgcaggagtcaggacctggcctggtggcgccctcacagag | LPDYGVSWIRQPPRKGL |
| | cctgtccgtcacatgcactgtctcaggggtctcattacccgac | EWLGVIWGSETTYYNS |
| | tatggtgtaagctggattcgccagcctccacgaaagggtctgg | ALKSRLTIIKDNSKSQVF |
| | agtggctgggagtaatatggggtagtgaaaccacatactataa | LKMNSLQTDDTAIYYCA |
| | ttcagctctcaaatccagactgaccatcatcaaggacaactcc | KHYYYGGSYAMDYWG |
| | aagagccaagttttcttaaaaatgaacagtctgcaaactgatg | QGTSVTVSSTTTPAPRP |
| | acacagccatttactactgtgccaaacattattactacggtgg | PTPAPTIASQPLSLRPEA |
| | tagctatgctatggactactggggccaaggaacctcagtcacc | CRPAAGGAVHTRGLDF |
| | cgcccaccatcgcgtcgcagcccctgtccctgcgcccagaggc | ACDIYIWAPLAGTCGVL |
| | gtctcctcaaccacgacgccagcgccgcgaccaccaacaccgg | LLSLVITLYCKRGRKKLLY |
| | gtgccggccagcggcggggggcgcagtgcacacgagggggctg | IFKQPFMRPVQTTQEE |
| | gacttcgcctgtgatatctacatctgggcgcccttggccggga | DGCSCRFPEEEEGGCEL |
| | cttgtggggtccttctcctgtcactggttatcaccctttactg | RVKFSRSADAPAYQQG |
| | caaacggggcagaaagaaactcctgtatatattcaaacaacca | QNQLYNELNLGRREEY |
| | tttatgagaccagtacaaactactcaagaggaagatggctgta | DVLDKRRGRDPEMGG |
| | gctgccgatttccagaagaagaagaaggaggatgtgaactgag | KPRRKNPQEGLYNELQ |
| | agtgaagttcagcaggagcgcagacgcccccgcgtaccagcag | KDKMAEAYSEIGMKGE |
| | ggccagaaccagctctataacgagctcaatctaggacgaagag | RRRGKGHDGLYQGLST |
| | aggagtacgatgttttggacaagagacgtggccgggaccctga | ATKDTYDALHMQALPP |
| | gatggggggaaagccgagaaggaagaaccctcaggaaggcctg | RGSGEGRGSLLTCGDVE |
| | tacaatgaactgcagaaagataagatggcggaggcctacagtg | ENPGPGTSLLCWMALC |
| | tggcctttaccagggtctcagtacagccaccaaggacacctac | LLGADHADAGIHVFILG |
| | agattgggatgaaaggcgagcgccggaggggcaaggggcacga | CFSAGLPKTEANWVNVI |
| | gacgcccttcacatgcaggccctgccccctcgcggaagtggag | SDLKKIEDLIQSMHIDAT |
| | agggcaggggaagtctactaacatgcggggacgtggaggaaaa | LYTESDVHPSCKVTAMK |
| | tcccggccccggcaccagcctgctgtgctggatggcactgtgc | CFLLELQVISLESGDASIH |
| | ctgctgggagcagaccacgccgatgccggcatccacgtgttca | DTVENLIILANNSLSSNG |
| | tcctgggctgcttcagcgccggacttcctaaaacagaggccaa | NVTESGCKECEELEEKNI |
| | ctgggtgaacgtgattagcgacctgaagaagatcgaggacctg | KEFLQSFVHIVQMFINT |
| | atgtgcatcctagctgcaaggtgaccgccatgaagtgcttcct | SRAEFLENGGTSLSEKTV |
| | atccagagcatgcacatcgacgccaccctgtacaccgagagcg | LLLVTPFLAAAWSLHPG |
| | attcacgacacagtggaaaatctgatcatcctggccaacaaca | SGATNFSLLKQAGDVEE |
| | gctggagctgcaggtgatcagcctggagagcggagatgccagc | NPGPAVMAPRTLLLLLS |
| | gcctgagcagcaacggcaatgtgaccgagagcggctgtaagga | GALALTQTWAIWELKK |
| | gtgcgaggaactggaggagaagaacatcaaggagttcctgcag | DVYVVELDWYPDAPGE |
| | agcttcgtgcacatcgtgcagatgttcatcaacaccagcagag | MVVLTCDTPEEDGITW |
| | ccgaattccttgaaaatggtgggacatccttatcagagaaaac | TLDQSSEVLGSGKTLTIQ |
| | agttcttctgctggtgactccatttctggcagcagcctggagc | VKEFGDAGQYTCHKGG |
| | cttcatcccggctccggcgccacgaacttctctctgttaaagc | EVLSHSLLLLHKKEDGIW |
| | ccctagaaccctgctgctgctgctgagcggcgccctggccctg | STDILKDQKEPKNKTFLR |
| | aagcaggagacgtggaagaaaaccccggtcccgctgtgatggc | CEAKNYSGRFTCWWLT |
| | acacagacctgggccatatgggaactgaagaaagatgtttatg | TISTDLTFSVKSSRGSSD |
| | tcgtagaattggattggtatccggatgcccctggagaaatggt | PQGVTCGAATLSAERV |
| | ggtcctcacctgtgacacccctgaagaagatggtatcacctgg | RGDNKEYEYSVECQEDS |
| | accttggaccagagcagtgaggtcttaggctctggcaaaaccc | ACPAAEESLPIEVMVDA |
| | tgaccatccaagtcaaagagtttggagatgctggccagtacac | VHKLKYENYTSSFFIRDII |
| | ctgtcacaaaggaggcgaggttctaagccattcgctcctgctg | KPDPPKNLQLKPLKNSR |
| | cttcacaaaaaggaagatggaatttggtccactgatattttaa | QVEVSWEYPDTWSTPH |
| | aggaccagaaagaacccaaaaataagacctttctaagatgcga | SYFSLTFCVQVQGKSKR |
| | ggccaagaattattctggacgtttcacctgctggtggctgacg | EKKDRVFTDKTSATVICR |
| | acaatcagtactgatttgacattcagtgtcaaaagcagcagag | KNASISVRAQDRYYSSS |
| | gctcttctgacccccaaggggtgacgtgcggagctgctacact | WSEWASVPCSRAEFLE |
| | ctctgcagagagagtcagaggggacaacaaggagtatgagtac | NGGTSLSEKTVLLLVTPF |
| | tcagtggagtgccaggaggacagtgcctgcccagctgctgagg | LAAAWSLHP (SEQ ID |
| | agagtctgcccattgaggtcatggtggatgccgttcacaagct | NO: 687) |
| | caagtatgaaaactacaccagcagcttcttcatcagggacatc | |
| | atcaaacctgacccacccaagaacttgcagctgaagccattaa | |
| | agaattctcggcaggtggaggtcagctgggagtaccctgacac | |
| | ctggagtactccacattcctacttctccctgacattctgcgtt | |
| | caggtccagggcaagagcaagagagaaaagaaagatagagtct | |
| | tcacggacaagacctcagccacggtcatctgccgcaaaaatgc | |
| | cagcattagcgtgcgggcccaggaccgctactatagctcatct | |
| | tggagcgaatgggcatctgtgccctgcagtagagccgagttcc | |
| | tggagaacggaggaacaagcctgagcgagaaaacagtgctgct | |
| | gctggtgaccccattcctggcagccgcctggagcctgcacccc | |
| | (SEQ ID NO: 676) | |
| CAR19-T2A- | gccttaccagtgaccgccttgctcctgccgctggccttgctgctccacgccgccaggccgg | ALPVTALLLPLALLLHAA |
| SP1-IL-21- | acatccagatgacacagactacatcctccctgtctgcctctctgggagacagagtcacca | RPDIQMTQTTSSLSASL |
| Anchoring | tcagttgcagggcaagtcaggacattagtaaatatttaaattggtatcagcagaaaccag | GDRVTISCRASQDISKYL |
| structure 1- | atggaactgttaaactcctgatctaccatacatcaagattacactcaggagtcccatcaa | NWYQQKPDGTVKLLIY |
| P2A-SP2-IL- | ggttcagtggcagtgggtctggaacagattattctctcaccattagcaacctggagcaag | HTSRLHSGVPSRFSGSG |
| 12p40- | aagatattgccacttacttttgccaacagggtaatacgcttccgtacacgttcggaggggg | SGTDYSLTISNLEQEDIA |
| Anchoring | gaccaagctggagatcacaggtggcggtggctcgggcggtggtgggtcgggtggcggc | TYFCQQGNTLPYTFGG |
| structure 2 | ggatctgaggtgaaactgcaggagtcaggacctggcctggtggcgccctcacagagcct | GTKLEITGGGGSGGGG |
| | gtccgtcacatgcactgtctcaggggtctcattacccgactatggtgtaagctggattcgc | SGGGGSEVKLQESGPG |
| | cagcctccacgaaagggtctggagtggctgggagtaatatggggtagtgaaaccacata | LVAPSQSLSVTCTVSGV |
| | ctataattcagctctcaaatccagactgaccatcatcaaggacaactccaagagccaagt | SLPDYGVSWIRQPPRKG |
| | tttcttaaaaatgaacagtctgcaaactgatgacacagccatttactactgtgccaaacat | LEWLGVIWGSETTYYNS |
| | tattactacggtggtagctatgctatggactactggggccaaggaacctcagtcaccgtct | ALKSRLTIIKDNSKSQVF |
| | cctcaaccacgacgccagcgccgcgaccaccaacaccggcgcccaccatcgcgtcgca | LKMNSLQTDDTAIYYCA |
| | gcccctgtccctgcgcccagaggcgtgccggccagcggcggggggcgcagtgcacacg | KHYYYGGSYAMDYWG |
| | agggggctggacttcgcctgtgatatctacatctgggcgcccttggccgggacttgtgggg | QGTSVTVSSTTTPAPRP |
| | tccttctcctgtcactggttatcaccctttactgcaaacggggcagaaagaaactcctgtat | PTPAPTIASQPLSLRPEA |
| | atattcaaacaaccatttatgagaccagtacaaactactcaagaggaagatggctgtag | CRPAAGGAVHTRGLDF |
| | ctgccgatttccagaagaagaagaaggaggatgtgaactgagagtgaagttcagcagg | ACDIYIWAPLAGTCGVL |
| | agcgcagacgcccccgcgtaccagcagggccagaaccagctctataacgagctcaatct | LLSLVITLYCKRGRKKLLY |
| | aggacgaagagaggagtacgatgttttggacaagagacgtggccgggaccctgagatg | IFKQPFMRPVQTTQEE |
| | gggggaaagccgagaaggaagaaccctcaggaaggcctgtacaatgaactgcagaaa | DGCSCRFPEEEEGGCEL |
| | gataagatggcggaggcctacagtgagattgggatgaaaggcgagcgccggaggggc | RVKFSRSADAPAYQQG |
| | aaggggcacgatggcctttaccagggtctcagtacagccaccaaggacacctacgacgc | QNQLYNELNLGRREEY |
| | ccttcacatgcaggccctgccccctcgcgctggaacgcgtggctccggcgagggcaggg | DVLDKRRGRDPEMGG |
| | gaagtcttctaacatgcggggacgtggaggaaaatcccggcccagctagcggaacaag | KPRRKNPQEGLYNELQ |
| | cctgctgtgctggatggctctgtgcctgctgggcgctgaccacgccgacgctcagggcca | KDKMAEAYSEIGMKGE |
| | ggacagacacatgatcagaatgagacagctgatcgacatcgtggaccagctgaagaac | RRRGKGHDGLYQGLST |
| | tacgtgaacgacctggtgcccgagttcctgcccgctcctgaagacgtggaaacaaactgt | ATKDTYDALHMQALPP |
| | gagtggagcgctttcagctgcttccagaaggcccagctgaagagcgccaataccggaaa | RAGTRGSGEGRGSLLTC |
| | caatgagagaatcatcaacgtgagcatcaagaagctgaagagaaagccccccagcacc | GDVEENPGPASGTSLLC |
| | aacgccggaagaagacaaaaacatagactgacctgccccagctgcgatagctacgaga | WMALCLLGADHADAQ |
| | aaaagccccccaaggagttcctggagagattcaagagcctgctgcagaagatgatccac | GQDRHMIRMRQLIDIV |
| | cagcacctgagcagcagaacccacggctctgaagatagcagagctagagccgaattcct | DQLKNYVNDLVPEFLPA |
| | ggagaacggagggacaagcctgagcgagaagacagtgctgctgctggtgaccccatttc | PEDVETNCEWSAFSCF |
| | tggccgcagcatggagcctgcaccccgctggaatcgatggcagcggcgccacaaacttc | QKAQLKSANTGNNERII |
| | tctctgctaaagcaagcaggtgatgttgaagaaaaccccgggcctctcgaggctgtgatg | NVSIKKLKRKPPSTNAG |
| | gcccctagaaccctgctgctgctgctgagcggcgccctggccctgacacagacctgggcc | RRQKHRLTCPSCDSYEK |
| | atatgggaactgaagaaagatgtttatgtcgtagaattggattggtatccggatgcccct | KPPKEFLERFKSLLQKMI |
| | ggagaaatggtggtcctcacctgtgacacccctgaagaagatggtatcacctggaccttg | HQHLSSRTHGSEDSRAR |
| | gaccagagcagtgaggtcttaggctctggcaaaaccctgaccatccaagtcaaagagtt | AEFLENGGTSLSEKTVLL |
| | tggagatgctggccagtacacctgtcacaaaggaggcgaggttctaagccattcgctcct | LVTPFLAAAWSLHPAGI |
| | gctgcttcacaaaaaggaagatggaatttggtccactgatattttaaaggaccagaaag | DGSGATNFSLLKQAGD |
| | aacccaaaaataagacctttctaagatgcgaggccaagaattattctggacgtttcacct | VEENPGPLEAVMAPRT |
| | gctggtggctgacgacaatcagtactgatttgacattcagtgtcaaaagcagcagaggct | LLLLLSGALALTQTWAI |
| | cttctgacccccaaggggtgacgtgcggagctgctacactctctgcagagagagtcaga | WELKKDVYVVELDWYP |
| | ggggacaacaaggagtatgagtactcagtggagtgccaggaggacagtgcctgcccag | DAPGEMVVLTCDTPEE |
| | ctgctgaggagagtctgcccattgaggtcatggtggatgccgttcacaagctcaagtatg | DGITWTLDQSSEVLGSG |
| | aaaactacaccagcagcttcttcatcagggacatcatcaaacctgacccacccaagaac | KTLTIQVKEFGDAGQYT |
| | ttgcagctgaagccattaaagaattctcggcaggtggaggtcagctgggagtaccctgac | CHKGGEVLSHSLLLLHK |
| | acctggagtactccacattcctacttctccctgacattctgcgttcaggtccagggcaaga | KEDGIWSTDILKDQKEP |
| | gcaagagagaaaagaaagatagagtcttcacggacaagacctcagccacggtcatctg | KNKTFLRCEAKNYSGRF |
| | ccgcaaaaatgccagcattagcgtgcgggcccaggaccgctactatagctcatcttggag | TCWWLTTISTDLTFSVK |
| | cgaatgggcatctgtgccctgcagtagagccgagttcctggagaacggaggaacaagcc | SSRGSSDPQGVTCGAA |
| | tgagcgagaaaacagtgctgctgctggtgaccccattcctggcagccgcctggagcctgc | TLSAERVRGDNKEYEYS |
| | acccc (SEQ ID NO: 677) | VECQEDSACPAAEESLPI |
| | | EVMVDAVHKLKYENYT |
| | | SSFFIRDIIKPDPPKNLQL |
| | | KPLKNSRQVEVSWEYP |
| | | DTWSTPHSYFSLTFCVQ |
| | | VQGKSKREKKDRVFTD |
| | | KTSATVICRKNASISVRA |
| | | QDRYYSSSWSEWASVP |
| | | CSRAEFLENGGTSLSEKT |
| | | VLLLVTPFLAAAWSLHP |
| | | (SEQ ID NO: 688) |
| SP1-IL-15- | ggcaccagcctgctgtgctggatggcactgtgcctgctgggagcagaccacgccgatgcc | GTSLLCWMALCLLGAD |
| Anchoring | ggcatccacgtgttcatcctgggctgcttcagcgccggacttcctaaaacagaggccaac | HADAGIHVFILGCFSAG |
| structure 1- | tgggtgaacgtgattagcgacctgaagaagatcgaggacctgatccagagcatgcacat | LPKTEANWVNVISDLKK |
| P2A-SP2-IL- | cgacgccaccctgtacaccgagagcgatgtgcatcctagctgcaaggtgaccgccatga | IEDLIQSMHIDATLYTES |
| 12p40- | agtgcttcctgctggagctgcaggtgatcagcctggagagcggagatgccagcattcacg | DVHPSCKVTAMKCFLLE |
| Anchoring | acacagtggaaaatctgatcatcctggccaacaacagcctgagcagcaacggcaatgtg | LQVISLESGDASIHDTVE |
| structure 2- | accgagagcggctgtaaggagtgcgaggaactggaggagaagaacatcaaggagttc | NLIILANNSLSSNGNVTE |
| T2A-CAR19 | ctgcagagcttcgtgcacatcgtgcagatgttcatcaacaccagcagagccgaattcctt | SGCKECEELEEKNIKEFL |
| | gaaaatggtgggacatccttatcagagaaaacagttcttctgctggtgactccatttctgg | QSFVHIVQMFINTSRAE |
| | cagcagcctggagccttcatcccggctccggcgccacgaacttctctctgttaaagcaag | FLENGGTSLSEKTVLLLV |
| | caggagacgtggaagaaaaccccggtcccgctgtgatggcccctagaaccctgctgctg | TPFLAAAWSLHPGSGA |
| | ctgctgagcggcgccctggccctgacacagacctgggccatatgggaactgaagaaaga | TNFSLLKQAGDVEENP |
| | tgtttatgtcgtagaattggattggtatccggatgcccctggagaaatggtggtcctcacct | GPAVMAPRTLLLLLSGA |
| | gtgacacccctgaagaagatggtatcacctggaccttggaccagagcagtgaggtctta | LALTQTWAIWELKKDV |
| | ggctctggcaaaaccctgaccatccaagtcaaagagtttggagatgctggccagtacac | YVVELDWYPDAPGEM |
| | ctgtcacaaaggaggcgaggttctaagccattcgctcctgctgcttcacaaaaaggaag | VVLTCDTPEEDGITWTL |
| | atggaatttggtccactgatattttaaaggaccagaaagaacccaaaaataagacctttc | DQSSEVLGSGKTLTIQV |
| | taagatgcgaggccaagaattattctggacgtttcacctgctggtggctgacgacaatca | KEFGDAGQYTCHKGGE |
| | gtactgatttgacattcagtgtcaaaagcagcagaggctcttctgacccccaaggggtga | VLSHSLLLLHKKEDGIWS |
| | cgtgcggagctgctacactctctgcagagagagtcagaggggacaacaaggagtatga | TDILKDQKEPKNKTFLRC |
| | gtactcagtggagtgccaggaggacagtgcctgcccagctgctgaggagagtctgccca | EAKNYSGRFTCWWLTT |
| | ttgaggtcatggtggatgccgttcacaagctcaagtatgaaaactacaccagcagcttctt | ISTDLTFSVKSSRGSSDP |
| | catcagggacatcatcaaacctgacccacccaagaacttgcagctgaagccattaaaga | QGVTCGAATLSAERVR |
| | attctcggcaggtggaggtcagctgggagtaccctgacacctggagtactccacattcct | GDNKEYEYSVECQEDSA |
| | acttctccctgacattctgcgttcaggtccagggcaagagcaagagagaaaagaaagat | CPAAEESLPIEVMVDAV |
| | agagtcttcacggacaagacctcagccacggtcatctgccgcaaaaatgccagcattag | HKLKYENYTSSFFIRDIIK |
| | cgtgcgggcccaggaccgctactatagctcatcttggagcgaatgggcatctgtgccctg | PDPPKNLQLKPLKNSRQ |
| | cagtagagccgagttcctggagaacggaggaacaagcctgagcgagaaaacagtgctg | VEVSWEYPDTWSTPHS |
| | ctgctggtgaccccattcctggcagccgcctggagcctgcaccccggaagtggagaggg | YFSLTFCVQVQGKSKRE |
| | caggggaagtctactaacatgcggggacgtggaggaaaatcccggcccctctcgctccg | KKDRVFTDKTSATVICR |
| | tggccttagctgtgctcgcgctactctctctttctggcctggaggctgacatccagatgaca | KNASISVRAQDRYYSSS |
| | cagactacatcctccctgtctgcctctctgggagacagagtcaccatcagttgcagggca | WSEWASVPCSRAEFLE |
| | agtcaggacattagtaaatatttaaattggtatcagcagaaaccagatggaactgttaaa | NGGTSLSEKTVLLLVTPF |
| | ctcctgatctaccatacatcaagattacactcaggagtcccatcaaggttcagtggcagtg | LAAAWSLHPGSGEGRG |
| | ggtctggaacagattattctctcaccattagcaacctggagcaagaagatattgccactt | SLLTCGDVEENPGPSRS |
| | acttttgccaacagggtaatacgcttccgtacacgttcggaggggggaccaagctggag | VALAVLALLSLSGLEADI |
| | atcacaggtggcggtggctcgggcggtggtgggtcgggtggcggcggatctgaggtgaa | QMTQTTSSLSASLGDR |
| | actgcaggagtcaggacctggcctggtggcgccctcacagagcctgtccgtcacatgcac | VTISCRASQDISKYLNW |
| | tgtctcaggggtctcattacccgactatggtgtaagctggattcgccagcctccacgaaag | YQQKPDGTVKLLIYHTS |
| | ggtctggagtggctgggagtaatatggggtagtgaaaccacatactataattcagctctc | RLHSGVPSRFSGSGSGT |
| | aaatccagactgaccatcatcaaggacaactccaagagccaagttttcttaaaaatgaa | DYSLTISNLEQEDIATYF |
| | cagtctgcaaactgatgacacagccatttactactgtgccaaacattattactacggtggt | CQQGNTLPYTFGGGTK |
| | agctatgctatggactactggggccaaggaacctcagtcaccgtctcctcaaccacgacg | LEITGGGGSGGGGSGG |
| | ccagcgccgcgaccaccaacaccggcgcccaccatcgcgtcgcagcccctgtccctgcg | GGSEVKLQESGPGLVAP |
| | cccagaggcgtgccggccagcggcggggggcgcagtgcacacgagggggctggacttc | SQSLSVTCTVSGVSLPD |
| | gcctgtgatatctacatctgggcgcccttggccgggacttgtggggtccttctcctgtcact | YGVSWIRQPPRKGLEW |
| | ggttatcaccctttactgcaaacggggcagaaagaaactcctgtatatattcaaacaacc | LGVIWGSETTYYNSALK |
| | atttatgagaccagtacaaactactcaagaggaagatggctgtagctgccgatttccaga | SRLTIIKDNSKSQVFLKM |
| | agaagaagaaggaggatgtgaactgagagtgaagttcagcaggagcgcagacgcccc | NSLQTDDTAIYYCAKHY |
| | cgcgtaccagcagggccagaaccagctctataacgagctcaatctaggacgaagagag | YYGGSYAMDYWGQGT |
| | gagtacgatgttttggacaagagacgtggccgggaccctgagatggggggaaagccga | SVTVSSTTTPAPRPPTPA |
| | gaaggaagaaccctcaggaaggcctgtacaatgaactgcagaaagataagatggcgg | PTIASQPLSLRPEACRPA |
| | aggcctacagtgagattgggatgaaaggcgagcgccggaggggcaaggggcacgatg | AGGAVHTRGLDFACDIY |
| | gcctttaccagggtctcagtacagccaccaaggacacctacgacgcccttcacatgcagg | IWAPLAGTCGVLLLSLVI |
| | ccctgccccctcgc (SEQ ID NO: 678) | TLYCKRGRKKLLYIFKQP |
| | | FMRPVQTTQEEDGCSC |
| | | RFPEEEEGGCELRVKFS |
| | | RSADAPAYQQGQNQLY |
| | | NELNLGRREEYDVLDKR |
| | | RGRDPEMGGKPRRKN |
| | | PQEGLYNELQKDKMAE |
| | | AYSEIGMKGERRRGKG |
| | | HDGLYQGLSTATKDTYD |
| | | ALHMQALPPR (SEQ |
| | | ID NO: 689) |
| SP1-IL-15- | ggaacaagcctgctgtgctggatggctctgtgcctgctgggcgctgaccacgccgacgct | GTSLLCWMALCLLGAD |
| Anchoring | ggcatccacgtgttcatcctgggctgcttcagcgccggacttcctaaaacagaggccaac | HADAGIHVFILGCFSAG |
| structure 1- | tgggtgaacgtgattagcgacctgaagaagatcgaggacctgatccagagcatgcacat | LPKTEANWVNVISDLKK |
| P2A-SP2-IL- | cgacgccaccctgtacaccgagagcgatgtgcatcctagctgcaaggtgaccgccatga | IEDLIQSMHIDATLYTES |
| 21- | agtgcttcctgctggagctgcaggtgatcagcctggagagcggagatgccagcattcacg | DVHPSCKVTAMKCFLLE |
| Anchoring | acacagtggaaaatctgatcatcctggccaacaacagcctgagcagcaacggcaatgtg | LQVISLESGDASIHDTVE |
| structure 2- | accgagagcggctgtaaggagtgcgaggaactggaggagaagaacatcaaggagttc | NLIILANNSLSSNGNVTE |
| T2A-CAR19 | ctgcagagcttcgtgcacatcgtgcagatgttcatcaacaccagcagagccagagccga | SGCKECEELEEKNIKEFL |
| | gtttctggagaacggagggacaagcctgagcgagaagacagtgctgctgctggtgaccc | QSFVHIVQMFINTSRAR |
| | catttctggccgcagcatggagcctgcaccccggctccggcgccacgaacttctctctgtt | AEFLENGGTSLSEKTVLL |
| | aaagcaagcaggagacgtggaagaaaaccccggtcccggcaccagcctgctgtgctgg | LVTPFLAAAWSLHPGSG |
| | atggcactgtgcctgctgggagcagaccacgccgatgcccagggccaggacagacaca | ATNFSLLKQAGDVEENP |
| | tgatcagaatgagacagctgatcgacatcgtggaccagctgaagaactacgtgaacgac | GPGTSLLCWMALCLLG |
| | ctggtgcccgagttcctgcccgctcctgaagacgtggaaacaaactgtgagtggagcgct | ADHADAQGQDRHMIR |
| | ttcagctgcttccagaaggcccagctgaagagcgccaataccggaaacaatgagagaat | MRQLIDIVDQLKNYVN |
| | catcaacgtgagcatcaagaagctgaagagaaagccccccagcaccaacgccggaag | DLVPEFLPAPEDVETNC |
| | aagacaaaaacatagactgacctgccccagctgcgatagctacgagaaaaagcccccc | EWSAFSCFQKAQLKSA |
| | aaggagttcctggagagattcaagagcctgctgcagaagatgatccaccagcacctgag | NTGNNERIINVSIKKLKR |
| | cagcagaacccacggctctgaagatagcagagctagagccgaattccttgaaaatggtg | KPPSTNAGRRQKHRLTC |
| | ggacatccttatcagagaaaacagttcttctgctggtgactccatttctggcagcagcctg | PSCDSYEKKPPKEFLERF |
| | gagccttcatcccggctccggcgagggcaggggaagtctactaacatgcggggacgtgg | KSLLQKMIHQHLSSRTH |
| | aggaaaatcccggccccgccttaccagtgaccgccttgctcctgccgctggccttgctgct | GSEDSRARAEFLENGGT |
| | ccacgccgccaggccggacatccagatgacacagactacatcctccctgtctgcctctct | SLSEKTVLLLVTPFLAAA |
| | gggagacagagtcaccatcagttgcagggcaagtcaggacattagtaaatatttaaatt | WSLHPGSGEGRGSLLTC |
| | ggtatcagcagaaaccagatggaactgttaaactcctgatctaccatacatcaagattac | GDVEENPGPALPVTALL |
| | actcaggagtcccatcaaggttcagtggcagtgggtctggaacagattattctctcaccat | LPLALLLHAARPDIQMT |
| | tagcaacctggagcaagaagatattgccacttacttttgccaacagggtaatacgcttcc | QTTSSLSASLGDRVTISC |
| | gtacacgttcggaggggggaccaagctggagatcacaggtggcggtggctcgggcggt | RASQDISKYLNWYQQK |
| | ggtgggtcgggtggcggcggatctgaggtgaaactgcaggagtcaggacctggcctggt | PDGTVKLLIYHTSRLHSG |
| | ggcgccctcacagagcctgtccgtcacatgcactgtctcaggggtctcattacccgactat | VPSRFSGSGSGTDYSLTI |
| | ggtgtaagctggattcgccagcctccacgaaagggtctggagtggctgggagtaatatg | SNLEQEDIATYFCQQGN |
| | gggtagtgaaaccacatactataattcagctctcaaatccagactgaccatcatcaagga | TLPYTFGGGTKLEITGG |
| | caactccaagagccaagttttcttaaaaatgaacagtctgcaaactgatgacacagccat | GGSGGGGSGGGGSEV |
| | ttactactgtgccaaacattattactacggtggtagctatgctatggactactggggccaa | KLQESGPGLVAPSQSLS |
| | ggaacctcagtcaccgtctcctcaaccacgacgccagcgccgcgaccaccaacaccggc | VTCTVSGVSLPDYGVS |
| | gcccaccatcgcgtcgcagcccctgtccctgcgcccagaggcgtgccggccagcggcgg | WIRQPPRKGLEWLGVI |
| | ggggcgcagtgcacacgagggggctggacttcgcctgtgatatctacatctgggcgccct | WGSETTYYNSALKSRLTI |
| | tggccgggacttgtggggtccttctcctgtcactggttatcaccctttactgcaaacggggc | IKDNSKSQVFLKMNSLQ |
| | agaaagaaactcctgtatatattcaaacaaccatttatgagaccagtacaaactactcaa | TDDTAIYYCAKHYYYGG |
| | gaggaagatggctgtagctgccgatttccagaagaagaagaaggaggatgtgaactga | SYAMDYWGQGTSVTV |
| | gagtgaagttcagcaggagcgcagacgcccccgcgtaccagcagggccagaaccagct | SSTTTPAPRPPTPAPTIA |
| | ctataacgagctcaatctaggacgaagagaggagtacgatgttttggacaagagacgtg | SQPLSLRPEACRPAAGG |
| | gccgggaccctgagatggggggaaagccgagaaggaagaaccctcaggaaggcctgt | AVHTRGLDFACDIYIWA |
| | acaatgaactgcagaaagataagatggcggaggcctacagtgagattgggatgaaagg | PLAGTCGVLLLSLVITLY |
| | cgagcgccggaggggcaaggggcacgatggcctttaccagggtctcagtacagccacc | CKRGRKKLLYIFKQPFM |
| | aaggacacctacgacgcccttcacatgcaggccctgccccctcgc (SEQ ID NO: | RPVQTTQEEDGCSCRFP |
| | 679) | EEEEGGCELRVKFSRSA |
| | | DAPAYQQGQNQLYNEL |
| | | NLGRREEYDVLDKRRGR |
| | | DPEMGGKPRRKNPQE |
| | | GLYNELQKDKMAEAYS |
| | | EIGMKGERRRGKGHDG |
| | | LYQGLSTATKDTYDALH |
| | | MQALPPR (SEQ ID |
| | | NO: 690) |
| SP1-IL-21- | ggaacaagcctgctgtgctggatggctctgtgcctgctgggcgctgaccacgccgacgct | GTSLLCWMALCLLGAD |
| Anchoring | cagggccaggacagacacatgatcagaatgagacagctgatcgacatcgtggaccagc | HADAQGQDRHMIRMR |
| structure 1- | tgaagaactacgtgaacgacctggtgcccgagttcctgcccgctcctgaagacgtggaa | QLIDIVDQLKNYVNDLV |
| P2A-SP2-IL- | acaaactgtgagtggagcgctttcagctgcttccagaaggcccagctgaagagcgccaa | PEFLPAPEDVETNCEWS |
| 15- | taccggaaacaatgagagaatcatcaacgtgagcatcaagaagctgaagagaaagccc | AFSCFQKAQLKSANTG |
| Anchoring | cccagcaccaacgccggaagaagacaaaaacatagactgacctgccccagctgcgata | NNERIINVSIKKLKRKPP |
| structure 2- | gctacgagaaaaagccccccaaggagttcctggagagattcaagagcctgctgcagaa | STNAGRRQKHRLTCPSC |
| T2A-CAR19 | gatgatccaccagcacctgagcagcagaacccacggctctgaagatagcagagctaga | DSYEKKPPKEFLERFKSL |
| | gccgagtttctggagaacggagggacaagcctgagcgagaagacagtgctgctgctggt | LQKMIHQHLSSRTHGSE |
| | gaccccatttctggccgcagcatggagcctgcaccccggatctggagcaacaaacttctc | DSRARAEFLENGGTSLS |
| | actactcaaacaagcaggtgacgtggaggagaatccggggcccggcaccagcctgctgt | EKTVLLLVTPFLAAAWS |
| | gctggatggcactgtgcctgctgggagcagaccacgccgatgccggcatccacgtgttca | LHPGSGATNFSLLKQAG |
| | tcctgggctgcttcagcgccggacttcctaaaacagaggccaactgggtgaacgtgatta | DVEENPGPGTSLLCWM |
| | gcgacctgaagaagatcgaggacctgatccagagcatgcacatcgacgccaccctgtac | ALCLLGADHADAGIHVF |
| | accgagagcgatgtgcatcctagctgcaaggtgaccgccatgaagtgcttcctgctggag | ILGCFSAGLPKTEANWV |
| | ctgcaggtgatcagcctggagagcggagatgccagcattcacgacacagtggaaaatct | NVISDLKKIEDLIQSMHI |
| | gatcatcctggccaacaacagcctgagcagcaacggcaatgtgaccgagagcggctgta | DATLYTESDVHPSCKVT |
| | aggagtgcgaggaactggaggagaagaacatcaaggagttcctgcagagcttcgtgca | AMKCFLLELQVISLESG |
| | catcgtgcagatgttcatcaacaccagcagagccagagccgaattccttgaaaatggtg | DASIHDTVENLIILANNS |
| | ggacatccttatcagagaaaacagttcttctgctggtgactccatttctggcagcagcctg | LSSNGNVTESGCKECEE |
| | gagccttcatcccggctccggcgagggcaggggaagtctactaacatgcggggacgtgg | LEEKNIKEFLQSFVHIVQ |
| | aggaaaatcccggccccgccttaccagtgaccgccttgctcctgccgctggccttgctgct | MFINTSRARAEFLENGG |
| | ccacgccgccaggccggacatccagatgacacagactacatcctccctgtctgcctctct | TSLSEKTVLLLVTPFLAA |
| | gggagacagagtcaccatcagttgcagggcaagtcaggacattagtaaatatttaaatt | AWSLHPGSGEGRGSLL |
| | ggtatcagcagaaaccagatggaactgttaaactcctgatctaccatacatcaagattac | TCGDVEENPGPALPVT |
| | actcaggagtcccatcaaggttcagtggcagtgggtctggaacagattattctctcaccat | ALLLPLALLLHAARPDIQ |
| | tagcaacctggagcaagaagatattgccacttacttttgccaacagggtaatacgcttcc | MTQTTSSLSASLGDRVT |
| | gtacacgttcggaggggggaccaagctggagatcacaggtggcggtggctcgggcggt | ISCRASQDISKYLNWYQ |
| | ggtgggtcgggtggcggcggatctgaggtgaaactgcaggagtcaggacctggcctggt | QKPDGTVKLLIYHTSRL |
| | ggcgccctcacagagcctgtccgtcacatgcactgtctcaggggtctcattacccgactat | HSGVPSRFSGSGSGTDY |
| | ggtgtaagctggattcgccagcctccacgaaagggtctggagtggctgggagtaatatg | SLTISNLEQEDIATYFCQ |
| | gggtagtgaaaccacatactataattcagctctcaaatccagactgaccatcatcaagga | QGNTLPYTFGGGTKLEI |
| | caactccaagagccaagttttcttaaaaatgaacagtctgcaaactgatgacacagccat | TGGGGSGGGGSGGGG |
| | ttactactgtgccaaacattattactacggtggtagctatgctatggactactggggccaa | SEVKLQESGPGLVAPSQ |
| | ggaacctcagtcaccgtctcctcaaccacgacgccagcgccgcgaccaccaacaccggc | SLSVTCTVSGVSLPDYG |
| | gcccaccatcgcgtcgcagcccctgtccctgcgcccagaggcgtgccggccagcggcgg | VSWIRQPPRKGLEWLG |
| | ggggcgcagtgcacacgagggggctggacttcgcctgtgatatctacatctgggcgccct | VIWGSETTYYNSALKSR |
| | tggccgggacttgtggggtccttctcctgtcactggttatcaccctttactgcaaacggggc | LTIIKDNSKSQVFLKMNS |
| | agaaagaaactcctgtatatattcaaacaaccatttatgagaccagtacaaactactcaa | LQTDDTAIYYCAKHYYY |
| | gaggaagatggctgtagctgccgatttccagaagaagaagaaggaggatgtgaactga | GGSYAMDYWGQGTSV |
| | gagtgaagttcagcaggagcgcagacgcccccgcgtaccagcagggccagaaccagct | TVSSTTTPAPRPPTPAPT |
| | ctataacgagctcaatctaggacgaagagaggagtacgatgttttggacaagagacgtg | IASQPLSLRPEACRPAA |
| | gccgggaccctgagatggggggaaagccgagaaggaagaaccctcaggaaggcctgt | GGAVHTRGLDFACDIYI |
| | acaatgaactgcagaaagataagatggcggaggcctacagtgagattgggatgaaagg | WAPLAGTCGVLLLSLVI |
| | cgagcgccggaggggcaaggggcacgatggcctttaccagggtctcagtacagccacc | TLYCKRGRKKLLYIFKQP |
| | aaggacacctacgacgcccttcacatgcaggccctgccccctcgc (SEQ ID NO: | FMRPVQTTQEEDGCSC |
| | 680) | RFPEEEEGGCELRVKFS |
| | | RSADAPAYQQGQNQLY |
| | | NELNLGRREEYDVLDKR |
| | | RGRDPEMGGKPRRKN |
| | | PQEGLYNELQKDKMAE |
| | | AYSEIGMKGERRRGKG |
| | | HDGLYQGLSTATKDTYD |
| | | ALHMQALPPR (SEQ |
| | | ID NO: 691) |
| SP1-IL- | gctgtgatggcccctagaaccctgctgctgctgctgagcggcgccctggccctgacacag | AVMAPRTLLLLLSGALA |
| 12p40- | acctgggccatatgggaactgaagaaagatgtttatgtcgtagaattggattggtatccg | LTQTWAIWELKKDVYV |
| Anchoring | gatgcccctggagaaatggtggtcctcacctgtgacacccctgaagaagatggtatcacc | VELDWYPDAPGEMVVL |
| structure 1- | tggaccttggaccagagcagtgaggtcttaggctctggcaaaaccctgaccatccaagtc | TCDTPEEDGITWTLDQS |
| P2A-SP2-IL- | aaagagtttggagatgctggccagtacacctgtcacaaaggaggcgaggttctaagcca | SEVLGSGKTLTIQVKEFG |
| 21- | ttcgctcctgctgcttcacaaaaaggaagatggaatttggtccactgatattttaaaggac | DAGQYTCHKGGEVLSH |
| Anchoring | cagaaagaacccaaaaataagacctttctaagatgcgaggccaagaattattctggacg | SLLLLHKKEDGIWSTDIL |
| structure 2- | tttcacctgctggtggctgacgacaatcagtactgatttgacattcagtgtcaaaagcagc | KDQKEPKNKTFLRCEAK |
| T2A-CAR19 | agaggctcttctgacccccaaggggtgacgtgcggagctgctacactctctgcagagag | NYSGRFTCWWLTTISTD |
| | agtcagaggggacaacaaggagtatgagtactcagtggagtgccaggaggacagtgcc | LTFSVKSSRGSSDPQGV |
| | tgcccagctgctgaggagagtctgcccattgaggtcatggtggatgccgttcacaagctc | TCGAATLSAERVRGDN |
| | aagtatgaaaactacaccagcagcttcttcatcagggacatcatcaaacctgacccaccc | KEYEYSVECQEDSACPA |
| | aagaacttgcagctgaagccattaaagaattctcggcaggtggaggtcagctgggagta | AEESLPIEVMVDAVHKL |
| | ccctgacacctggagtactccacattcctacttctccctgacattctgcgttcaggtccagg | KYENYTSSFFIRDIIKPDP |
| | gcaagagcaagagagaaaagaaagatagagtcttcacggacaagacctcagccacgg | PKNLQLKPLKNSRQVEV |
| | tcatctgccgcaaaaatgccagcattagcgtgcgggcccaggaccgctactatagctcat | SWEYPDTWSTPHSYFSL |
| | cttggagcgaatgggcatctgtgccctgcagtagagccgagttcctggagaacggagga | TFCVQVQGKSKREKKD |
| | acaagcctgagcgagaaaacagtgctgctgctggtgaccccattcctggcagccgcctg | RVFTDKTSATVICRKNA |
| | gagcctgcaccccggaagtggagccacgaacttctctctgttaaagcaagcaggagacg | SISVRAQDRYYSSSWSE |
| | tggaagaaaaccccggtcccggcaccagcctgctgtgctggatggcactgtgcctgctgg | WASVPCSRAEFLENGG |
| | gagcagaccacgccgatgcccagggccaggacagacacatgatcagaatgagacagct | TSLSEKTVLLLVTPFLAA |
| | gatcgacatcgtggaccagctgaagaactacgtgaacgacctggtgcccgagttcctgcc | AWSLHPGSGATNFSLLK |
| | cgctcctgaagacgtggaaacaaactgtgagtggagcgctttcagctgcttccagaagg | QAGDVEENPGPGTSLL |
| | cccagctgaagagcgccaataccggaaacaatgagagaatcatcaacgtgagcatcaa | CWMALCLLGADHADA |
| | gaagctgaagagaaagccccccagcaccaacgccggaagaagacaaaaacatagact | QGQDRHMIRMRQLIDI |
| | gacctgccccagctgcgatagctacgagaaaaagccccccaaggagttcctggagagat | VDQLKNYVNDLVPEFLP |
| | tcaagagcctgctgcagaagatgatccaccagcacctgagcagcagaacccacggctct | APEDVETNCEWSAFSC |
| | gaagatagcagagctagagccgaattccttgaaaatggtgggacatccttatcagagaa | FQKAQLKSANTGNNERI |
| | aacagttcttctgctggtgactccatttctggcagcagcctggagccttcatcccggctccg | INVSIKKLKRKPPSTNAG |
| | gcgagggcaggggaagtctactaacatgcggggacgtggaggaaaatcccggccccgc | RRQKHRLTCPSCDSYEK |
| | cttaccagtgaccgccttgctcctgccgctggccttgctgctccacgccgccaggccggac | KPPKEFLERFKSLLQKMI |
| | atccagatgacacagactacatcctccctgtctgcctctctgggagacagagtcaccatc | HQHLSSRTHGSEDSRAR |
| | agttgcagggcaagtcaggacattagtaaatatttaaattggtatcagcagaaaccagat | AEFLENGGTSLSEKTVLL |
| | ggaactgttaaactcctgatctaccatacatcaagattacactcaggagtcccatcaagg | LVTPFLAAAWSLHPGSG |
| | ttcagtggcagtgggtctggaacagattattctctcaccattagcaacctggagcaagaa | EGRGSLLTCGDVEENPG |
| | gatattgccacttacttttgccaacagggtaatacgcttccgtacacgttcggagggggga | PALPVTALLLPLALLLHA |
| | ccaagctggagatcacaggtggcggtggctcgggcggtggtgggtcgggtggcggcgg | ARPDIQMTQTTSSLSAS |
| | atctgaggtgaaactgcaggagtcaggacctggcctggtggcgccctcacagagcctgt | LGDRVTISCRASQDISKY |
| | ccgtcacatgcactgtctcaggggtctcattacccgactatggtgtaagctggattcgcca | LNWYQQKPDGTVKLLIY |
| | gcctccacgaaagggtctggagtggctgggagtaatatggggtagtgaaaccacatact | HTSRLHSGVPSRFSGSG |
| | ataattcagctctcaaatccagactgaccatcatcaaggacaactccaagagccaagttt | SGTDYSLTISNLEQEDIA |
| | tcttaaaaatgaacagtctgcaaactgatgacacagccatttactactgtgccaaacatt | TYFCQQGNTLPYTFGG |
| | attactacggtggtagctatgctatggactactggggccaaggaacctcagtcaccgtct | GTKLEITGGGGSGGGG |
| | cctcaaccacgacgccagcgccgcgaccaccaacaccggcgcccaccatcgcgtcgca | SGGGGSEVKLQESGPG |
| | gcccctgtccctgcgcccagaggcgtgccggccagcggcggggggcgcagtgcacacg | LVAPSQSLSVTCTVSGV |
| | agggggctggacttcgcctgtgatatctacatctgggcgcccttggccgggacttgtgggg | SLPDYGVSWIRQPPRKG |
| | tccttctcctgtcactggttatcaccctttactgcaaacggggcagaaagaaactcctgtat | LEWLGVIWGSETTYYNS |
| | atattcaaacaaccatttatgagaccagtacaaactactcaagaggaagatggctgtag | ALKSRLTIIKDNSKSQVF |
| | ctgccgatttccagaagaagaagaaggaggatgtgaactgagagtgaagttcagcagg | LKMNSLQTDDTAIYYCA |
| | agcgcagacgcccccgcgtaccagcagggccagaaccagctctataacgagctcaatct | KHYYYGGSYAMDYWG |
| | aggacgaagagaggagtacgatgttttggacaagagacgtggccgggaccctgagatg | QGTSVTVSSTTTPAPRP |
| | gggggaaagccgagaaggaagaaccctcaggaaggcctgtacaatgaactgcagaaa | PTPAPTIASQPLSLRPEA |
| | gataagatggcggaggcctacagtgagattgggatgaaaggcgagcgccggaggggc | CRPAAGGAVHTRGLDF |
| | aaggggcacgatggcctttaccagggtctcagtacagccaccaaggacacctacgacgc | ACDIYIWAPLAGTCGVL |
| | ccttcacatgcaggccctgccccctcgc (SEQ ID NO: 681) | LLSLVITLYCKRGRKKLLY |
| | | IFKQPFMRPVQTTQEE |
| | | DGCSCRFPEEEEGGCEL |
| | | RVKFSRSADAPAYQQG |
| | | QNQLYNELNLGRREEY |
| | | DVLDKRRGRDPEMGG |
| | | KPRRKNPQEGLYNELQ |
| | | KDKMAEAYSEIGMKGE |
| | | RRRGKGHDGLYQGLST |
| | | ATKDTYDALHMQALPP |
| | | R (SEQ ID NO: 692) |
| SP1-IL-21- | ggcaccagcctgctgtgctggatggcactgtgcctgctgggagcagaccacgccgatgcc | GTSLLCWMALCLLGAD |
| Anchoring | cagggccaggacagacacatgatcagaatgagacagctgatcgacatcgtggaccagc | HADAQGQDRHMIRMR |
| structure 1- | tgaagaactacgtgaacgacctggtgcccgagttcctgcccgctcctgaagacgtggaa | QLIDIVDQLKNYVNDLV |
| P2A-SP2-IL- | acaaactgtgagtggagcgctttcagctgcttccagaaggcccagctgaagagcgccaa | PEFLPAPEDVETNCEWS |
| 12p40- | taccggaaacaatgagagaatcatcaacgtgagcatcaagaagctgaagagaaagccc | AFSCFQKAQLKSANTG |
| Anchoring | cccagcaccaacgccggaagaagacaaaaacatagactgacctgccccagctgcgata | NNERIINVSIKKLKRKPP |
| structure 2- | gctacgagaaaaagccccccaaggagttcctggagagattcaagagcctgctgcagaa | STNAGRRQKHRLTCPSC |
| T2A-CAR19 | gatgatccaccagcacctgagcagcagaacccacggctctgaagatagcagagctaga | DSYEKKPPKEFLERFKSL |
| | gccgaattccttgaaaatggtgggacatccttatcagagaaaacagttcttctgctggtga | LQKMIHQHLSSRTHGSE |
| | ctccatttctggcagcagcctggagccttcatcccggatctggagcaacaaacttctcact | DSRARAEFLENGGTSLS |
| | actcaaacaagcaggtgacgtggaggagaatccggggcccgctgtgatggcccctaga | EKTVLLLVTPFLAAAWS |
| | accctgctgctgctgctgagcggcgccctggccctgacacagacctgggccatatgggaa | LHPGSGATNFSLLKQAG |
| | ctgaagaaagatgtttatgtcgtagaattggattggtatccggatgcccctggagaaatg | DVEENPGPAVMAPRTL |
| | gtggtcctcacctgtgacacccctgaagaagatggtatcacctggaccttggaccagagc | LLLLSGALALTQTWAIW |
| | agtgaggtcttaggctctggcaaaaccctgaccatccaagtcaaagagtttggagatgct | ELKKDVYVVELDWYPD |
| | ggccagtacacctgtcacaaaggaggcgaggttctaagccattcgctcctgctgcttcac | APGEMVVLTCDTPEED |
| | aaaaaggaagatggaatttggtccactgatattttaaaggaccagaaagaacccaaaa | GITWTLDQSSEVLGSGK |
| | ataagacctttctaagatgcgaggccaagaattattctggacgtttcacctgctggtggct | TLTIQVKEFGDAGQYTC |
| | gacgacaatcagtactgatttgacattcagtgtcaaaagcagcagaggctcttctgaccc | HKGGEVLSHSLLLLHKK |
| | ccaaggggtgacgtgcggagctgctacactctctgcagagagagtcagaggggacaac | EDGIWSTDILKDQKEPK |
| | aaggagtatgagtactcagtggagtgccaggaggacagtgcctgcccagctgctgagga | NKTFLRCEAKNYSGRFT |
| | gagtctgcccattgaggtcatggtggatgccgttcacaagctcaagtatgaaaactacac | CWWLTTISTDLTFSVKS |
| | cagcagcttcttcatcagggacatcatcaaacctgacccacccaagaacttgcagctgaa | SRGSSDPQGVTCGAAT |
| | gccattaaagaattctcggcaggtggaggtcagctgggagtaccctgacacctggagta | LSAERVRGDNKEYEYSV |
| | ctccacattcctacttctccctgacattctgcgttcaggtccagggcaagagcaagagag | ECQEDSACPAAEESLPIE |
| | aaaagaaagatagagtcttcacggacaagacctcagccacggtcatctgccgcaaaaa | VMVDAVHKLKYENYTS |
| | tgccagcattagcgtgcgggcccaggaccgctactatagctcatcttggagcgaatgggc | SFFIRDIIKPDPPKNLQLK |
| | atctgtgccctgcagtagagccgagttcctggagaacggaggaacaagcctgagcgaga | PLKNSRQVEVSWEYPD |
| | aaacagtgctgctgctggtgaccccattcctggcagccgcctggagcctgcaccccggaa | TWSTPHSYFSLTFCVQV |
| | gtggagagggcaggggaagtctactaacatgcggggacgtggaggaaaatcccggccc | QGKSKREKKDRVFTDKT |
| | cgccttaccagtgaccgccttgctcctgccgctggccttgctgctccacgccgccaggccg | SATVICRKNASISVRAQ |
| | gacatccagatgacacagactacatcctccctgtctgcctctctgggagacagagtcacc | DRYYSSSWSEWASVPC |
| | atcagttgcagggcaagtcaggacattagtaaatatttaaattggtatcagcagaaacca | SRAEFLENGGTSLSEKTV |
| | gatggaactgttaaactcctgatctaccatacatcaagattacactcaggagtcccatca | LLLVTPFLAAAWSLHPG |
| | aggttcagtggcagtgggtctggaacagattattctctcaccattagcaacctggagcaa | SGEGRGSLLTCGDVEEN |
| | gaagatattgccacttacttttgccaacagggtaatacgcttccgtacacgttcggagggg | PGPALPVTALLLPLALLL |
| | ggaccaagctggagatcacaggtggcggtggctcgggcggtggtgggtcgggtggcgg | HAARPDIQMTQTTSSLS |
| | cggatctgaggtgaaactgcaggagtcaggacctggcctggtggcgccctcacagagcc | ASLGDRVTISCRASQDIS |
| | tgtccgtcacatgcactgtctcaggggtctcattacccgactatggtgtaagctggattcgc | KYLNWYQQKPDGTVKL |
| | cagcctccacgaaagggtctggagtggctgggagtaatatggggtagtgaaaccacata | LIYHTSRLHSGVPSRFSG |
| | ctataattcagctctcaaatccagactgaccatcatcaaggacaactccaagagccaagt | SGSGTDYSLTISNLEQED |
| | tttcttaaaaatgaacagtctgcaaactgatgacacagccatttactactgtgccaaacat | IATYFCQQGNTLPYTFG |
| | tattactacggtggtagctatgctatggactactggggccaaggaacctcagtcaccgtct | GGTKLEITGGGGSGGG |
| | cctcaaccacgacgccagcgccgcgaccaccaacaccggcgcccaccatcgcgtcgca | GSGGGGSEVKLQESGP |
| | gcccctgtccctgcgcccagaggcgtgccggccagcggcggggggcgcagtgcacacg | GLVAPSQSLSVTCTVSG |
| | agggggctggacttcgcctgtgatatctacatctgggcgcccttggccgggacttgtgggg | VSLPDYGVSWIRQPPRK |
| | tccttctcctgtcactggttatcaccctttactgcaaacggggcagaaagaaactcctgtat | GLEWLGVIWGSETTYY |
| | atattcaaacaaccatttatgagaccagtacaaactactcaagaggaagatggctgtag | NSALKSRLTIIKDNSKSQ |
| | ctgccgatttccagaagaagaagaaggaggatgtgaactgagagtgaagttcagcagg | VFLKMNSLQTDDTAIYY |
| | agcgcagacgcccccgcgtaccagcagggccagaaccagctctataacgagctcaatct | CAKHYYYGGSYAMDY |
| | aggacgaagagaggagtacgatgttttggacaagagacgtggccgggaccctgagatg | WGQGTSVTVSSTTTPA |
| | gggggaaagccgagaaggaagaaccctcaggaaggcctgtacaatgaactgcagaaa | PRPPTPAPTIASQPLSLR |
| | gataagatggcggaggcctacagtgagattgggatgaaaggcgagcgccggaggggc | PEACRPAAGGAVHTRG |
| | aaggggcacgatggcctttaccagggtctcagtacagccaccaaggacacctacgacgc | LDFACDIYIWAPLAGTC |
| | ccttcacatgcaggccctgccccctcgc (SEQ ID NO: 682) | GVLLLSLVITLYCKRGRK |
| | | KLLYIFKQPFMRPVQTT |
| | | QEEDGCSCRFPEEEEGG |
| | | CELRVKFSRSADAPAYQ |
| | | QGQNQLYNELNLGRRE |
| | | EYDVLDKRRGRDPEMG |
| | | GKPRRKNPQEGLYNEL |
| | | QKDKMAEAYSEIGMKG |
| | | ERRRGKGHDGLYQGLS |
| | | TATKDTYDALHMQALP |
| | | PR (SEQ ID NO: 693) |
| SP1-IL- | gctgtgatggcccctagaaccctgctgctgctgctgagcggcgccctggccctgacacag | AVMAPRTLLLLLSGALA |
| 12p40- | acctgggccatatgggaactgaagaaagatgtttatgtcgtagaattggattggtatccg | LTQTWAIWELKKDVYV |
| Anchoring | gatgcccctggagaaatggtggtcctcacctgtgacacccctgaagaagatggtatcacc | VELDWYPDAPGEMVVL |
| structure 1- | tggaccttggaccagagcagtgaggtcttaggctctggcaaaaccctgaccatccaagtc | TCDTPEEDGITWTLDQS |
| T2A-SP2-IL- | aaagagtttggagatgctggccagtacacctgtcacaaaggaggcgaggttctaagcca | SEVLGSGKTLTIQVKEFG |
| 21- | ttcgctcctgctgcttcacaaaaaggaagatggaatttggtccactgatattttaaaggac | DAGQYTCHKGGEVLSH |
| Anchoring | cagaaagaacccaaaaataagacctttctaagatgcgaggccaagaattattctggacg | SLLLLHKKEDGIWSTDIL |
| structure 2- | tttcacctgctggtggctgacgacaatcagtactgatttgacattcagtgtcaaaagcagc | KDQKEPKNKTFLRCEAK |
| P2A-CAR19 | agaggctcttctgacccccaaggggtgacgtgcggagctgctacactctctgcagagag | NYSGRFTCWWLTTISTD |
| | agtcagaggggacaacaaggagtatgagtactcagtggagtgccaggaggacagtgcc | LTFSVKSSRGSSDPQGV |
| | tgcccagctgctgaggagagtctgcccattgaggtcatggtggatgccgttcacaagctc | TCGAATLSAERVRGDN |
| | aagtatgaaaactacaccagcagcttcttcatcagggacatcatcaaacctgacccaccc | KEYEYSVECQEDSACPA |
| | aagaacttgcagctgaagccattaaagaattctcggcaggtggaggtcagctgggagta | AEESLPIEVMVDAVHKL |
| | ccctgacacctggagtactccacattcctacttctccctgacattctgcgttcaggtccagg | KYENYTSSFFIRDIIKPDP |
| | gcaagagcaagagagaaaagaaagatagagtcttcacggacaagacctcagccacgg | PKNLQLKPLKNSRQVEV |
| | tcatctgccgcaaaaatgccagcattagcgtgcgggcccaggaccgctactatagctcat | SWEYPDTWSTPHSYFSL |
| | cttggagcgaatgggcatctgtgccctgcagtagagccgagttcctggagaacggagga | TFCVQVQGKSKREKKD |
| | acaagcctgagcgagaaaacagtgctgctgctggtgaccccattcctggcagccgcctg | RVFTDKTSATVICRKNA |
| | gagcctgcaccccgctggaacgcgtggctccggcgagggcaggggaagtcttctaacat | SISVRAQDRYYSSSWSE |
| | gcggggacgtggaggaaaatcccggcccagctagcggaacaagcctgctgtgctggat | WASVPCSRAEFLENGG |
| | ggctctgtgcctgctgggcgctgaccacgccgacgctcagggccaggacagacacatga | TSLSEKTVLLLVTPFLAA |
| | tcagaatgagacagctgatcgacatcgtggaccagctgaagaactacgtgaacgacctg | AWSLHPAGTRGSGEGR |
| | gtgcccgagttcctgcccgctcctgaagacgtggaaacaaactgtgagtggagcgctttc | GSLLTCGDVEENPGPAS |
| | agctgcttccagaaggcccagctgaagagcgccaataccggaaacaatgagagaatca | GTSLLCWMALCLLGAD |
| | tcaacgtgagcatcaagaagctgaagagaaagccccccagcaccaacgccggaagaa | HADAQGQDRHMIRMR |
| | gacaaaaacatagactgacctgccccagctgcgatagctacgagaaaaagccccccaa | QLIDIVDQLKNYVNDLV |
| | ggagttcctggagagattcaagagcctgctgcagaagatgatccaccagcacctgagca | PEFLPAPEDVETNCEWS |
| | gcagaacccacggctctgaagatagcagagctagagccgaattcctggagaacggagg | AFSCFQKAQLKSANTG |
| | gacaagcctgagcgagaagacagtgctgctgctggtgaccccatttctggccgcagcat | NNERIINVSIKKLKRKPP |
| | ggagcctgcaccccgctggaatcgatggcagcggcgccacaaacttctctctgctaaagc | STNAGRRQKHRLTCPSC |
| | aagcaggtgatgttgaagaaaaccccgggcctctcgaggccttaccagtgaccgccttgc | DSYEKKPPKEFLERFKSL |
| | tcctgccgctggccttgctgctccacgccgccaggccggacatccagatgacacagacta | LQKMIHQHLSSRTHGSE |
| | catcctccctgtctgcctctctgggagacagagtcaccatcagttgcagggcaagtcagg | DSRARAEFLENGGTSLS |
| | acattagtaaatatttaaattggtatcagcagaaaccagatggaactgttaaactcctga | EKTVLLLVTPFLAAAWS |
| | tctaccatacatcaagattacactcaggagtcccatcaaggttcagtggcagtgggtctg | LHPAGIDGSGATNFSLL |
| | gaacagattattctctcaccattagcaacctggagcaagaagatattgccacttacttttg | KQAGDVEENPGPLEAL |
| | ccaacagggtaatacgcttccgtacacgttcggaggggggaccaagctggagatcacag | PVTALLLPLALLLHAARP |
| | gtggcggtggctcgggcggtggtgggtcgggtggcggcggatctgaggtgaaactgcag | DIQMTQTTSSLSASLGD |
| | gagtcaggacctggcctggtggcgccctcacagagcctgtccgtcacatgcactgtctca | RVTISCRASQDISKYLN |
| | ggggtctcattacccgactatggtgtaagctggattcgccagcctccacgaaagggtctg | WYQQKPDGTVKLLIYH |
| | gagtggctgggagtaatatggggtagtgaaaccacatactataattcagctctcaaatcc | TSRLHSGVPSRFSGSGS |
| | agactgaccatcatcaaggacaactccaagagccaagttttcttaaaaatgaacagtctg | GTDYSLTISNLEQEDIAT |
| | caaactgatgacacagccatttactactgtgccaaacattattactacggtggtagctatg | YFCQQGNTLPYTFGGG |
| | ctatggactactggggccaaggaacctcagtcaccgtctcctcaaccacgacgccagcg | TKLEITGGGGSGGGGS |
| | ccgcgaccaccaacaccggcgcccaccatcgcgtcgcagcccctgtccctgcgcccaga | GGGGSEVKLQESGPGL |
| | ggcgtgccggccagcggcggggggcgcagtgcacacgagggggctggacttcgcctgt | VAPSQSLSVTCTVSGVS |
| | gatatctacatctgggcgcccttggccgggacttgtggggtccttctcctgtcactggttat | LPDYGVSWIRQPPRKGL |
| | caccctttactgcaaacggggcagaaagaaactcctgtatatattcaaacaaccatttat | EWLGVIWGSETTYYNS |
| | gagaccagtacaaactactcaagaggaagatggctgtagctgccgatttccagaagaag | ALKSRLTIIKDNSKSQVF |
| | aagaaggaggatgtgaactgagagtgaagttcagcaggagcgcagacgcccccgcgta | LKMNSLQTDDTAIYYCA |
| | ccagcagggccagaaccagctctataacgagctcaatctaggacgaagagaggagtac | KHYYYGGSYAMDYWG |
| | gatgttttggacaagagacgtggccgggaccctgagatggggggaaagccgagaagga | QGTSVTVSSTTTPAPRP |
| | agaaccctcaggaaggcctgtacaatgaactgcagaaagataagatggcggaggccta | PTPAPTIASQPLSLRPEA |
| | cagtgagattgggatgaaaggcgagcgccggaggggcaaggggcacgatggcctttac | CRPAAGGAVHTRGLDF |
| | cagggtctcagtacagccaccaaggacacctacgacgcccttcacatgcaggccctgcc | ACDIYIWAPLAGTCGVL |
| | ccctcgc (SEQ ID NO: 683) | LLSLVITLYCKRGRKKLLY |
| | | IFKQPFMRPVQTTQEE |
| | | DGCSCRFPEEEEGGCEL |
| | | RVKFSRSADAPAYQQG |
| | | QNQLYNELNLGRREEY |
| | | DVLDKRRGRDPEMGG |
| | | KPRRKNPQEGLYNELQ |
| | | KDKMAEAYSEIGMKGE |
| | | RRRGKGHDGLYQGLST |
| | | ATKDTYDALHMQALPP |
| | | R (SEQ ID NO: 694) |
| SP1-IL-21- | ggaacaagcctgctgtgctggatggctctgtgcctgctgggcgctgaccacgccgacgct | GTSLLCWMALCLLGAD |
| Anchoring | cagggccaggacagacacatgatcagaatgagacagctgatcgacatcgtggaccagc | HADAQGQDRHMIRMR |
| structure 1- | tgaagaactacgtgaacgacctggtgcccgagttcctgcccgctcctgaagacgtggaa | QLIDIVDQLKNYVNDLV |
| T2A-SP2-IL- | acaaactgtgagtggagcgctttcagctgcttccagaaggcccagctgaagagcgccaa | PEFLPAPEDVETNCEWS |
| 12p40- | taccggaaacaatgagagaatcatcaacgtgagcatcaagaagctgaagagaaagccc | AFSCFQKAQLKSANTG |
| Anchoring | cccagcaccaacgccggaagaagacaaaaacatagactgacctgccccagctgcgata | NNERIINVSIKKLKRKPP |
| structure 2- | gctacgagaaaaagccccccaaggagttcctggagagattcaagagcctgctgcagaa | STNAGRRQKHRLTCPSC |
| P2A-CAR19 | gatgatccaccagcacctgagcagcagaacccacggctctgaagatagcagagctaga | DSYEKKPPKEFLERFKSL |
| | gccgaattcctggagaacggagggacaagcctgagcgagaagacagtgctgctgctgg | LQKMIHQHLSSRTHGSE |
| | tgaccccatttctggccgcagcatggagcctgcaccccgctggaacgcgtggctccggcg | DSRARAEFLENGGTSLS |
| | agggcaggggaagtcttctaacatgcggggacgtggaggaaaatcccggcccagctag | EKTVLLLVTPFLAAAWS |
| | cgctgtgatggcccctagaaccctgctgctgctgctgagcggcgccctggccctgacaca | LHPAGTRGSGEGRGSLL |
| | gacctgggccatatgggaactgaagaaagatgtttatgtcgtagaattggattggtatcc | TCGDVEENPGPASAVM |
| | ggatgcccctggagaaatggtggtcctcacctgtgacacccctgaagaagatggtatcac | APRTLLLLLSGALALTQT |
| | ctggaccttggaccagagcagtgaggtcttaggctctggcaaaaccctgaccatccaagt | WAIWELKKDVYVVELD |
| | caaagagtttggagatgctggccagtacacctgtcacaaaggaggcgaggttctaagcc | WYPDAPGEMVVLTCD |
| | attcgctcctgctgcttcacaaaaaggaagatggaatttggtccactgatattttaaagga | TPEEDGITWTLDQSSEV |
| | ccagaaagaacccaaaaataagacctttctaagatgcgaggccaagaattattctggac | LGSGKTLTIQVKEFGDA |
| | gtttcacctgctggtggctgacgacaatcagtactgatttgacattcagtgtcaaaagcag | GQYTCHKGGEVLSHSLL |
| | cagaggctcttctgacccccaaggggtgacgtgcggagctgctacactctctgcagagag | LLHKKEDGIWSTDILKD |
| | agtcagaggggacaacaaggagtatgagtactcagtggagtgccaggaggacagtgcc | QKEPKNKTFLRCEAKNY |
| | tgcccagctgctgaggagagtctgcccattgaggtcatggtggatgccgttcacaagctc | SGRFTCWWLTTISTDLT |
| | aagtatgaaaactacaccagcagcttcttcatcagggacatcatcaaacctgacccaccc | FSVKSSRGSSDPQGVTC |
| | aagaacttgcagctgaagccattaaagaattctcggcaggtggaggtcagctgggagta | GAATLSAERVRGDNKE |
| | ccctgacacctggagtactccacattcctacttctccctgacattctgcgttcaggtccagg | YEYSVECQEDSACPAAE |
| | gcaagagcaagagagaaaagaaagatagagtcttcacggacaagacctcagccacgg | ESLPIEVMVDAVHKLKY |
| | tcatctgccgcaaaaatgccagcattagcgtgcgggcccaggaccgctactatagctcat | ENYTSSFFIRDIIKPDPPK |
| | cttggagcgaatgggcatctgtgccctgcagtagagccgagttcctggagaacggagga | NLQLKPLKNSRQVEVS |
| | acaagcctgagcgagaaaacagtgctgctgctggtgaccccattcctggcagccgcctg | WEYPDTWSTPHSYFSLT |
| | gagcctgcaccccgctggaatcgatggcagcggcgccacaaacttctctctgctaaagca | FCVQVQGKSKREKKDR |
| | agcaggtgatgttgaagaaaaccccgggcctctcgaggccttaccagtgaccgccttgct | VFTDKTSATVICRKNASI |
| | cctgccgctggccttgctgctccacgccgccaggccggacatccagatgacacagactac | SVRAQDRYYSSSWSEW |
| | atcctccctgtctgcctctctgggagacagagtcaccatcagttgcagggcaagtcagga | ASVPCSRAEFLENGGTS |
| | cattagtaaatatttaaattggtatcagcagaaaccagatggaactgttaaactcctgatc | LSEKTVLLLVTPFLAAA |
| | taccatacatcaagattacactcaggagtcccatcaaggttcagtggcagtgggtctgga | WSLHPAGIDGSGATNF |
| | acagattattctctcaccattagcaacctggagcaagaagatattgccacttacttttgcc | SLLKQAGDVEENPGPLE |
| | aacagggtaatacgcttccgtacacgttcggaggggggaccaagctggagatcacaggt | ALPVTALLLPLALLLHAA |
| | ggcggtggctcgggcggtggtgggtcgggtggcggcggatctgaggtgaaactgcagga | RPDIQMTQTTSSLSASL |
| | gtcaggacctggcctggtggcgccctcacagagcctgtccgtcacatgcactgtctcagg | GDRVTISCRASQDISKYL |
| | ggtctcattacccgactatggtgtaagctggattcgccagcctccacgaaagggtctgga | NWYQQKPDGTVKLLIY |
| | gtggctgggagtaatatggggtagtgaaaccacatactataattcagctctcaaatccag | HTSRLHSGVPSRFSGSG |
| | actgaccatcatcaaggacaactccaagagccaagttttcttaaaaatgaacagtctgca | SGTDYSLTISNLEQEDIA |
| | aactgatgacacagccatttactactgtgccaaacattattactacggtggtagctatgct | TYFCQQGNTLPYTFGG |
| | atggactactggggccaaggaacctcagtcaccgtctcctcaaccacgacgccagcgcc | GTKLEITGGGGSGGGG |
| | gcgaccaccaacaccggcgcccaccatcgcgtcgcagcccctgtccctgcgcccagagg | SGGGGSEVKLQESGPG |
| | cgtgccggccagcggcggggggcgcagtgcacacgagggggctggacttcgcctgtgat | LVAPSQSLSVTCTVSGV |
| | atctacatctgggcgcccttggccgggacttgtggggtccttctcctgtcactggttatcac | SLPDYGVSWIRQPPRKG |
| | cctttactgcaaacggggcagaaagaaactcctgtatatattcaaacaaccatttatgag | LEWLGVIWGSETTYYNS |
| | accagtacaaactactcaagaggaagatggctgtagctgccgatttccagaagaagaa | ALKSRLTIIKDNSKSQVF |
| | gaaggaggatgtgaactgagagtgaagttcagcaggagcgcagacgcccccgcgtacc | LKMNSLQTDDTAIYYCA |
| | agcagggccagaaccagctctataacgagctcaatctaggacgaagagaggagtacga | KHYYYGGSYAMDYWG |
| | tgttttggacaagagacgtggccgggaccctgagatggggggaaagccgagaaggaag | QGTSVTVSSTTTPAPRP |
| | aaccctcaggaaggcctgtacaatgaactgcagaaagataagatggcggaggcctaca | PTPAPTIASQPLSLRPEA |
| | gtgagattgggatgaaaggcgagcgccggaggggcaaggggcacgatggcctttacca | CRPAAGGAVHTRGLDF |
| | gggtctcagtacagccaccaaggacacctacgacgcccttcacatgcaggccctgcccc | ACDIYIWAPLAGTCGVL |
| | ctcgc (SEQ ID NO: 684) | LLSLVITLYCKRGRKKLLY |
| | | IFKQPFMRPVQTTQEE |
| | | DGCSCRFPEEEEGGCEL |
| | | RVKFSRSADAPAYQQG |
| | | QNQLYNELNLGRREEY |
| | | DVLDKRRGRDPEMGG |
| | | KPRRKNPQEGLYNELQ |
| | | KDKMAEAYSEIGMKGE |
| | | RRRGKGHDGLYQGLST |
| | | ATKDTYDALHMQALPP |
| | | R (SEQ ID NO: 695) |
| SP1-IL- | gctgtgatggcccctagaaccctgctgctgctgctgagcggcgccctggccctgacacag | AVMAPRTLLLLLSGALA |
| 12p40- | acctgggccatatgggaactgaagaaagatgtttatgtcgtagaattggattggtatccg | LTQTWAIWELKKDVYV |
| Anchoring | gatgcccctggagaaatggtggtcctcacctgtgacacccctgaagaagatggtatcacc | VELDWYPDAPGEMVVL |
| structure 1- | tggaccttggaccagagcagtgaggtcttaggctctggcaaaaccctgaccatccaagtc | TCDTPEEDGITWTLDQS |
| T2A-CAR19- | aaagagtttggagatgctggccagtacacctgtcacaaaggaggcgaggttctaagcca | SEVLGSGKTLTIQVKEFG |
| P2A-SP2-IL- | ttcgctcctgctgcttcacaaaaaggaagatggaatttggtccactgatattttaaaggac | DAGQYTCHKGGEVLSH |
| 21- | cagaaagaacccaaaaataagacctttctaagatgcgaggccaagaattattctggacg | SLLLLHKKEDGIWSTDIL |
| Anchoring | tttcacctgctggtggctgacgacaatcagtactgatttgacattcagtgtcaaaagcagc | KDQKEPKNKTFLRCEAK |
| structure 2 | agaggctcttctgacccccaaggggtgacgtgcggagctgctacactctctgcagagag | NYSGRFTCWWLTTISTD |
| | agtcagaggggacaacaaggagtatgagtactcagtggagtgccaggaggacagtgcc | LTFSVKSSRGSSDPQGV |
| | tgcccagctgctgaggagagtctgcccattgaggtcatggtggatgccgttcacaagctc | TCGAATLSAERVRGDN |
| | aagtatgaaaactacaccagcagcttcttcatcagggacatcatcaaacctgacccaccc | KEYEYSVECQEDSACPA |
| | aagaacttgcagctgaagccattaaagaattctcggcaggtggaggtcagctgggagta | AEESLPIEVMVDAVHKL |
| | ccctgacacctggagtactccacattcctacttctccctgacattctgcgttcaggtccagg | KYENYTSSFFIRDIIKPDP |
| | gcaagagcaagagagaaaagaaagatagagtcttcacggacaagacctcagccacgg | PKNLQLKPLKNSRQVEV |
| | tcatctgccgcaaaaatgccagcattagcgtgcgggcccaggaccgctactatagctcat | SWEYPDTWSTPHSYFSL |
| | cttggagcgaatgggcatctgtgccctgcagtagagccgagttcctggagaacggagga | TFCVQVQGKSKREKKD |
| | acaagcctgagcgagaaaacagtgctgctgctggtgaccccattcctggcagccgcctg | RVFTDKTSATVICRKNA |
| | gagcctgcaccccgctggaacgcgtggctccggcgagggcaggggaagtcttctaacat | SISVRAQDRYYSSSWSE |
| | gcggggacgtggaggaaaatcccggcccagctagcgccttaccagtgaccgccttgctc | WASVPCSRAEFLENGG |
| | ctgccgctggccttgctgctccacgccgccaggccggacatccagatgacacagactaca | TSLSEKTVLLLVTPFLAA |
| | tcctccctgtctgcctctctgggagacagagtcaccatcagttgcagggcaagtcaggac | AWSLHPAGTRGSGEGR |
| | attagtaaatatttaaattggtatcagcagaaaccagatggaactgttaaactcctgatct | GSLLTCGDVEENPGPAS |
| | accatacatcaagattacactcaggagtcccatcaaggttcagtggcagtgggtctggaa | ALPVTALLLPLALLLHAA |
| | cagattattctctcaccattagcaacctggagcaagaagatattgccacttacttttgcca | RPDIQMTQTTSSLSASL |
| | acagggtaatacgcttccgtacacgttcggaggggggaccaagctggagatcacaggtg | GDRVTISCRASQDISKYL |
| | gcggtggctcgggcggtggtgggtcgggtggcggcggatctgaggtgaaactgcaggag | NWYQQKPDGTVKLLIY |
| | tcaggacctggcctggtggcgccctcacagagcctgtccgtcacatgcactgtctcaggg | HTSRLHSGVPSRFSGSG |
| | gtctcattacccgactatggtgtaagctggattcgccagcctccacgaaagggtctggag | SGTDYSLTISNLEQEDIA |
| | tggctgggagtaatatggggtagtgaaaccacatactataattcagctctcaaatccaga | TYFCQQGNTLPYTFGG |
| | ctgaccatcatcaaggacaactccaagagccaagttttcttaaaaatgaacagtctgcaa | GTKLEITGGGGSGGGG |
| | actgatgacacagccatttactactgtgccaaacattattactacggtggtagctatgcta | SGGGGSEVKLQESGPG |
| | tggactactggggccaaggaacctcagtcaccgtctcctcaaccacgacgccagcgccg | LVAPSQSLSVTCTVSGV |
| | cgaccaccaacaccggcgcccaccatcgcgtcgcagcccctgtccctgcgcccagaggc | SLPDYGVSWIRQPPRKG |
| | gtgccggccagcggcggggggcgcagtgcacacgagggggctggacttcgcctgtgata | LEWLGVIWGSETTYYNS |
| | tctacatctgggcgcccttggccgggacttgtggggtccttctcctgtcactggttatcacc | ALKSRLTIIKDNSKSQVF |
| | ctttactgcaaacggggcagaaagaaactcctgtatatattcaaacaaccatttatgaga | LKMNSLQTDDTAIYYCA |
| | ccagtacaaactactcaagaggaagatggctgtagctgccgatttccagaagaagaag | KHYYYGGSYAMDYWG |
| | aaggaggatgtgaactgagagtgaagttcagcaggagcgcagacgcccccgcgtacca | QGTSVTVSSTTTPAPRP |
| | gcagggccagaaccagctctataacgagctcaatctaggacgaagagaggagtacgat | PTPAPTIASQPLSLRPEA |
| | gttttggacaagagacgtggccgggaccctgagatggggggaaagccgagaaggaag | CRPAAGGAVHTRGLDF |
| | aaccctcaggaaggcctgtacaatgaactgcagaaagataagatggcggaggcctaca | ACDIYIWAPLAGTCGVL |
| | gtgagattgggatgaaaggcgagcgccggaggggcaaggggcacgatggcctttacca | LLSLVITLYCKRGRKKLLY |
| | gggtctcagtacagccaccaaggacacctacgacgcccttcacatgcaggccctgcccc | IFKQPFMRPVQTTQEE |
| | ctcgcgctggaatcgatggcagcggcgccacaaacttctctctgctaaagcaagcaggtg | DGCSCRFPEEEEGGCEL |
| | atgttgaagaaaaccccgggcctctcgagggaacaagcctgctgtgctggatggctctgt | RVKFSRSADAPAYQQG |
| | gcctgctgggcgctgaccacgccgacgctcagggccaggacagacacatgatcagaat | QNQLYNELNLGRREEY |
| | gagacagctgatcgacatcgtggaccagctgaagaactacgtgaacgacctggtgcccg | DVLDKRRGRDPEMGG |
| | agttcctgcccgctcctgaagacgtggaaacaaactgtgagtggagcgctttcagctgct | KPRRKNPQEGLYNELQ |
| | tccagaaggcccagctgaagagcgccaataccggaaacaatgagagaatcatcaacgt | KDKMAEAYSEIGMKGE |
| | gagcatcaagaagctgaagagaaagccccccagcaccaacgccggaagaagacaaaa | RRRGKGHDGLYQGLST |
| | acatagactgacctgccccagctgcgatagctacgagaaaaagccccccaaggagttcc | ATKDTYDALHMQALPP |
| | tggagagattcaagagcctgctgcagaagatgatccaccagcacctgagcagcagaac | RAGIDGSGATNFSLLKQ |
| | ccacggctctgaagatagcagagctagagccgaattcctggagaacggagggacaagc | AGDVEENPGPLEGTSLL |
| | ctgagcgagaagacagtgctgctgctggtgaccccatttctggccgcagcatggagcctg | CWMALCLLGADHADA |
| | cacccc (SEQ ID NO: 685) | QGQDRHMIRMRQLIDI |
| | | VDQLKNYVNDLVPEFLP |
| | | APEDVETNCEWSAFSC |
| | | FQKAQLKSANTGNNERI |
| | | INVSIKKLKRKPPSTNAG |
| | | RRQKHRLTCPSCDSYEK |
| | | KPPKEFLERFKSLLQKMI |
| | | HQHLSSRTHGSEDSRAR |
| | | AEFLENGGTSLSEKTVLL |
| | | LVTPFLAAAWSLHP |
| | | (SEQ ID NO: 696) |
| SP1-IL-21- | ggaacaagcctgctgtgctggatggctctgtgcctgctgggcg | GTSLLCWMALCLLGA |
| Anchoring | ctgaccacgccgacgctcagggccaggacagacacatgatcag | DHADAQGQDRHMIRM |
| structure 1- | aatgagacagctgatcgacatcgtggaccagctgaagaactac | RQLIDIVDQLKNYVN |
| T2A-CAR19- | gtgaacgacctggtgcccgagttcctgcccgctcctgaagacg | DLVPEFLPAPEDVET |
| P2A-SP2-IL- | tggaaacaaactgtgagtggagcgctttcagctgcttccagaa | NCEWSAFSCFQKAQL |
| 12p40- | ggcccagctgaagagcgccaataccggaaacaatgagagaatc | KSANTGNNERIINVS |
| Anchoring | atcaacgtgagcatcaagaagctgaagagaaagccccccagca | IKKLKRKPPSTNAGR |
| structure 2 | ccaacgccggaagaagacaaaaacatagactgacctgccccag | RQKHRLTCPSCDSYE |
| | ctgcgatagctacgagaaaaagccccccaaggagttcctggag | KKPPKEFLERFKSLL |
| | agattcaagagcctgctgcagaagatgatccaccagcacctga | QKMIHQHLSSRTHGS |
| | gcagcagaacccacggctctgaagatagcagagctagagccga | EDSRARAEFLENGGT |
| | attcctggagaacggagggacaagcctgagcgagaagacagtg | SLSEKTVLLLVTPFL |
| | ctgctgctggtgaccccatttctggccgcagcatggagcctgc | AAAWSLHPAGTRGSG |
| | accccgctggaacgcgtggctccggcgagggcaggggaagtct | EGRGSLLTCGDVEEN |
| | tctaacatgcggggacgtggaggaaaatcccggcccagctagc | PGPASALPVTALLLP |
| | gccttaccagtgaccgccttgctcctgccgctggccttgctgc | LALLLHAARPDIQMT |
| | tccacgccgccaggccggacatccagatgacacagactacatc | QTTSSLSASLGDRVT |
| | ctccctgtctgcctctctgggagacagagtcaccatcagttgc | ISCRASQDISKYLNW |
| | agggcaagtcaggacattagtaaatatttaaattggtatcagc | YQQKPDGTVKLLIYH |
| | agaaaccagatggaactgttaaactcctgatctaccatacatc | TSRLHSGVPSRFSGS |
| | aagattacactcaggagtcccatcaaggttcagtggcagtggg | GSGTDYSLTISNLEQ |
| | tctggaacagattattctctcaccattagcaacctggagcaag | EDIATYFCQQGNTLP |
| | aagatattgccacttacttttgccaacagggtaatacgcttcc | YTFGGGTKLEITGGG |
| | gtacacgttcggaggggggaccaagctggagatcacaggtggc | GSGGGGSGGGGSEVK |
| | ggtggctcgggcggtggtgggtcgggtggcggcggatctgagg | LQESGPGLVAPSQSL |
| | tgaaactgcaggagtcaggacctggcctggtggcgccctcaca | SVTCTVSGVSLPDYG |
| | gagcctgtccgtcacatgcactgtctcaggggtctcattaccc | VSWIRQPPRKGLEWL |
| | gactatggtgtaagctggattcgccagcctccacgaaagggtc | GVIWGSETTYYNSAL |
| | tggagtggctgggagtaatatggggtagtgaaaccacatacta | KSRLTIIKDNSKSQV |
| | taattcagctctcaaatccagactgaccatcatcaaggacaac | FLKMNSLQTDDTAIY |
| | tccaagagccaagttttcttaaaaatgaacagtctgcaaactg | YCAKHYYYGGSYAMD |
| | atgacacagccatttactactgtgccaaacattattactacgg | YWGQGTSVTVSSTTT |
| | tggtagctatgctatggactactggggccaaggaacctcagtc | PAPRPPTPAPTIASQ |
| | accgtctcctcaaccacgacgccagcgccgcgaccaccaacac | PLSLRPEACRPAAGG |
| | cggcgcccaccatcgcgtcgcagcccctgtccctgcgcccaga | AVHTRGLDFACDIYI |
| | ggcgtgccggccagcggcggggggcgcagtgcacacgaggggg | WAPLAGTCGVLLLSL |
| | ctggacttcgcctgtgatatctacatctgggcgcccttggccg | VITLYCKRGRKKLLY |
| | ggacttgtggggtccttctcctgtcactggttatcacccttta | IFKQPFMRPVQTTQE |
| | ctgcaaacggggcagaaagaaactcctgtatatattcaaacaa | EDGCSCRFPEEEEGG |
| | ccatttatgagaccagtacaaactactcaagaggaagatggct | CELRVKFSRSADAPA |
| | gtagctgccgatttccagaagaagaagaaggaggatgtgaact | YQQGQNQLYNELNLG |
| | gagagtgaagttcagcaggagcgcagacgcccccgcgtaccag | RREEYDVLDKRRGRD |
| | cagggccagaaccagctctataacgagctcaatctaggacgaa | PEMGGKPRRKNPQEG |
| | gagaggagtacgatgttttggacaagagacgtggccgggaccc | LYNELQKDKMAEAYS |
| | tgagatggggggaaagccgagaaggaagaaccctcaggaaggc | EIGMKGERRRGKGHD |
| | ctgtacaatgaactgcagaaagataagatggcggaggcctaca | GLYQGLSTATKDTYD |
| | gtgagattgggatgaaaggcgagcgccggaggggcaaggggca | ALHMQALPPRAGIDG |
| | cgatggcctttaccagggtctcagtacagccaccaaggacacc | SGATNFSLLKQAGDV |
| | tacgacgcccttcacatgcaggccctgccccctcgcgctggaa | EENPGPLEAVMAPRT |
| | tcgatggcagcggcgccacaaacttctctctgctaaagcaagc | LLLLLSGALALTQTW |
| | aggtgatgttgaagaaaaccccgggcctctcgaggctgtgatg | AIWELKKDVYVVELD |
| | gcccctagaaccctgctgctgctgctgagcggcgccctggccc | WYPDAPGEMVVLTCD |
| | tgacacagacctgggccatatgggaactgaagaaagatgttta | TPEEDGITWTLDQSS |
| | tgtcgtagaattggattggtatccggatgcccctggagaaatg | EVLGSGKTLTIQVKE |
| | gtggtcctcacctgtgacacccctgaagaagatggtatcacct | FGDAGQYTCHKGGEV |
| | ggaccttggaccagagcagtgaggtcttaggctctggcaaaac | LSHSLLLLHKKEDGI |
| | cctgaccatccaagtcaaagagtttggagatgctggccagtac | WSTDILKDQKEPKNK |
| | acctgtcacaaaggaggcgaggttctaagccattcgctcctgc | TFLRCEAKNYSGRFT |
| | tgcttcacaaaaaggaagatggaatttggtccactgatatttt | CWWLTTISTDLTFSV |
| | aaaggaccagaaagaacccaaaaataagacctttctaagatgc | KSSRGSSDPQGVTCG |
| | gaggccaagaattattctggacgtttcacctgctggtggctga | AATLSAERVRGDNKE |
| | cgacaatcagtactgatttgacattcagtgtcaaaagcagcag | YEYSVECQEDSACPA |
| | aggctcttctgacccccaaggggtgacgtgcggagctgctaca | AEESLPIEVMVDAVH |
| | ctctctgcagagagagtcagaggggacaacaaggagtatgagt | KLKYENYTSSFFIRD |
| | actcagtggagtgccaggaggacagtgcctgcccagctgctga | IIKPDPPKNLQLKPL |
| | ggagagtctgcccattgaggtcatggtggatgccgttcacaag | KNSRQVEVSWEYPDT |
| | ctcaagtatgaaaactacaccagcagcttcttcatcagggaca | WSTPHSYFSLTFCVQ |
| | tcatcaaacctgacccacccaagaacttgcagctgaagccatt | VQGKSKREKKDRVFT |
| | aaagaattctcggcaggtggaggtcagctgggagtaccctgac | DKTSATVICRKNASI |
| | acctggagtactccacattcctacttctccctgacattctgcg | SVRAQDRYYSSSWSE |
| | ttcaggtccagggcaagagcaagagagaaaagaaagatagagt | WASVPCSRAEFLENG |
| | cttcacggacaagacctcagccacggtcatctgccgcaaaaat | GTSLSEKTVLLLVTP |
| | gccagcattagcgtgcgggcccaggaccgctactatagctcat | FLAAAWSLHP (SEQ |
| | cttggagcgaatgggcatctgtgccctgcagtagagccgagtt | ID NO: 697) |
| | cctggagaacggaggaacaagcctgagcgagaaaacagtgctg | |
| | ctgctggtgaccccattcctggcagccgcctggagcctgcacc | |
| | cc (SEQ ID NO: 686) | |

TABLE A-7
VIRAL VECTOR CONSTRUCTS WITH CAR AND THREE CYTOKINES

General structure 1: SP1-Cytokine1-Anchoring structure 1-
Linker-SP2-Cytokine2-Anchoring structure
2-Linker-SP3-Cytokine3-Anchoring structure 3-Linker-CAR19
General structure 2: CAR19-Linker-SP1-Cytokine1-Anchoring structure
1-Linker-SP2-Cytokine2-Anchoring structure 2-Linker-SP3-Cytokine3-
Anchoring structure 3
General structure 3: SP1-Cytokine1-Anchoring structure 1-Linker-CAR19-
Linker-SP2-Cytokine2-Anchoring structure 2-Linker-SP3-Cytokine3-
Anchoring structure 3
General structure 4: SP1-Cytokine1-Anchoring structure 1-Linker-SP2-
Cytokine2-Anchoring structure 2-Linker-CAR19-Linker-SP3-Cytokine3-
Anchoring structure 3
SP1-IL-21-Anchoring structure 1-T2A-SP2-IL-12p40-Anchoring structure 2-
P2A-IL-15-Anchoring structure 3-T2A-CAR19
Nucleic Acid Sequence
ggaacaagcctgctgtgctggatggctctgtgcctgctgggcgctgaccacgccgacgctcagggcca
ggacagacacatgatcagaatgagacagctgatcgacatcgtggaccagctgaagaactacgtgaacg
acctggtgcccgagttcctgcccgctcctgaagacgtggaaacaaactgtgagtggagcgctttcagc
tgcttccagaaggcccagctgaagagcgccaataccggaaacaatgagagaatcatcaacgtgagcat
caagaagctgaagagaaagccccccagcaccaacgccggaagaagacaaaaacatagactgacctgcc
ccagctgcgatagctacgagaaaaagccccccaaggagttcctggagagattcaagagcctgctgcag
aagatgatccaccagcacctgagcagcagaacccacggctctgaagatagcagagctagagccgaatt
cctggagaacggagggacaagcctgagcgagaagacagtgctgctgctggtgaccccatttctggccg
cagcatggagcctgcaccccgctggaacgcgtggctccggcgagggcaggggaagtcttctaacatgc
ggggacgtggaggaaaatcccggcccagctagcgctgtgatggcccctagaaccctgctgctgctgct
gagcggcgccctggccctgacacagacctgggccatatgggaactgaagaaagatgtttatgtcgtag
aattggattggtatccggatgcccctggagaaatggtggtcctcacctgtgacacccctgaagaagat
ggtatcacctggaccttggaccagagcagtgaggtcttaggctctggcaaaaccctgaccatccaagt
caaagagtttggagatgctggccagtacacctgtcacaaaggaggcgaggttctaagccattcgctcc
tgctgcttcacaaaaaggaagatggaatttggtccactgatattttaaaggaccagaaagaacccaaa
aataagacctttctaagatgcgaggccaagaattattctggacgtttcacctgctggtggctgacgac
aatcagtactgatttgacattcagtgtcaaaagcagcagaggctcttctgacccccaaggggtgacgt
gcggagctgctacactctctgcagagagagtcagaggggacaacaaggagtatgagtactcagtggag
tgccaggaggacagtgcctgcccagctgctgaggagagtctgcccattgaggtcatggtggatgccgt
tcacaagctcaagtatgaaaactacaccagcagcttcttcatcagggacatcatcaaacctgacccac
ccaagaacttgcagctgaagccattaaagaattctcggcaggtggaggtcagctgggagtaccctgac
acctggagtactccacattcctacttctccctgacattctgcgttcaggtccagggcaagagcaagag
agaaaagaaagatagagtcttcacggacaagacctcagccacggtcatctgccgcaaaaatgccagca
ttagcgtgcgggcccaggaccgctactatagctcatcttggagcgaatgggcatctgtgccctgcagt
agagccgagttcctggagaacggaggaacaagcctgagcgagaaaacagtgctgctgctggtgacccc
attcctggcagccgcctggagcctgcaccccgctggaatcgatggcagcggcgccacaaacttctctc
tgctaaagcaagcaggtgatgttgaagaaaaccccgggcctctcgagggcaccagcctgctgtgctgg
atggcactgtgcctgctgggagcagaccacgccgatgccggcatccacgtgttcatcctgggctgctt
cagcgccggacttcctaaaacagaggccaactgggtgaacgtgattagcgacctgaagaagatcgagg
acctgatccagagcatgcacatcgacgccaccctgtacaccgagagcgatgtgcatcctagctgcaag
gtgaccgccatgaagtgcttcctgctggagctgcaggtgatcagcctggagagcggagatgccagcat
tcacgacacagtggaaaatctgatcatcctggccaacaacagcctgagcagcaacggcaatgtgaccg
agagcggctgtaaggagtgcgaggaactggaggagaagaacatcaaggagttcctgcagagcttcgtg
cacatcgtgcagatgttcatcaacaccagcagagccagggccgagttccttgaaaatggtgggacatc
cttatcagagaaaacagttcttctgctggtgactccatttctggcagcagcctggagccttcatcccg
gctccggcgagggcaggggaagtctactaacatgcggggacgtggaggaaaatcccggccccgcctta
ccagtgaccgccttgctcctgccgctggccttgctgctccacgccgccaggccggacatccagatgac
acagactacatcctccctgtctgcctctctgggagacagagtcaccatcagttgcagggcaagtcagg
acattagtaaatatttaaattggtatcagcagaaaccagatggaactgttaaactcctgatctaccat
acatcaagattacactcaggagtcccatcaaggttcagtggcagtgggtctggaacagattattctct
caccattagcaacctggagcaagaagatattgccacttacttttgccaacagggtaatacgcttccgt
acacgttcggaggggggaccaagctggagatcacaggtggcggtggctcgggcggtggtgggtcgggt
ggcggcggatctgaggtgaaactgcaggagtcaggacctggcctggtggcgccctcacagagcctgtc
cgtcacatgcactgtctcaggggtctcattacccgactatggtgtaagctggattcgccagcctccac
gaaagggtctggagtggctgggagtaatatggggtagtgaaaccacatactataattcagctctcaaa
tccagactgaccatcatcaaggacaactccaagagccaagttttcttaaaaatgaacagtctgcaaac
tgatgacacagccatttactactgtgccaaacattattactacggtggtagctatgctatggactact
ggggccaaggaacctcagtcaccgtctcctcaaccacgacgccagcgccgcgaccaccaacaccggcg
cccaccatcgcgtcgcagcccctgtccctgcgcccagaggcgtgccggccagcggcggggggcgcagt
gcacacgagggggctggacttcgcctgtgatatctacatctgggcgcccttggccgggacttgtgggg
tccttctcctgtcactggttatcaccctttactgcaaacggggcagaaagaaactcctgtatatattc
aaacaaccatttatgagaccagtacaaactactcaagaggaagatggctgtagctgccgatttccaga
agaagaagaaggaggatgtgaactgagagtgaagttcagcaggagcgcagacgcccccgcgtaccagc
agggccagaaccagctctataacgagctcaatctaggacgaagagaggagtacgatgttttggacaag
agacgtggccgggaccctgagatggggggaaagccgagaaggaagaaccctcaggaaggcctgtacaa
tgaactgcagaaagataagatggcggaggcctacagtgagattgggatgaaaggcgagcgccggaggg
gcaaggggcacgatggcctttaccagggtctcagtacagccaccaaggacacctacgacgcccttcac
atgcaggccctgccccctcgcgttttcttaaaaatgaacagtctgcaaactgatgacacagccattta
ctactgtgccaaacattattactacggtggtagctatgctatggactactggggccaaggaacctcag
tcaccgtctcctcaaccacgacgccagcgccgcgaccaccaacaccggcgcccaccatcgcgtcgcag
cccctgtccctgcgcccagaggcgtgccggccagcggcggggggcgcagtgcacacgagggggctgga
cttcgcctgtgatatctacatctgggcgcccttggccgggacttgtggggtccttctcctgtcactgg
ttatcaccctttactgcaaacggggcagaaagaaactcctgtatatattcaaacaaccatttatgaga
ccagtacaaactactcaagaggaagatggctgtagctgccgatttccagaagaagaagaaggaggatg
tgaactgagagtgaagttcagcaggagcgcagacgcccccgcgtaccagcagggccagaaccagctct
ataacgagctcaatctaggacgaagagaggagtacgatgttttggacaagagacgtggccgggaccct
gagatggggggaaagccgagaaggaagaaccctcaggaaggcctgtacaatgaactgcagaaagataa
gatggcggaggcctacagtgagattgggatgaaaggcgagcgccggaggggcaaggggcacgatggcc
tttaccagggtctcagtacagccaccaaggacacctacgacgcccttcacatgcaggccctgccccct
cgc (SEQ ID NO: 698)
Amino Acid sequence
GTSLLCWMALCLLGADHADAQGQDRHMIRMRQLIDIVDQLKNYVNDLVPEFLPAPEDVETNCEWSAFS
CFQKAQLKSANTGNNERIINVSIKKLKRKPPSTNAGRROKHRLTCPSCDSYEKKPPKEFLERFKSLLQ
KMIHQHLSSRTHGSEDSRARAEFLENGGTSLSEKTVLLLVTPFLAAAWSLHPAGTRGSGEGRGSLLTC
GDVEENPGPASAVMAPRTLLLLLSGALALTQTWAIWELKKDVYVVELDWYPDAPGEMVVLTCDTPEED
GITWTLDQSSEVLGSGKTLTIQVKEFGDAGQYTCHKGGEVLSHSLLLLHKKEDGIWSTDILKDQKEPK
NKTFLRCEAKNYSGRFTCWWLTTISTDLTFSVKSSRGSSDPQGVTCGAATLSAERVRGDNKEYEYSVE
CQEDSACPAAEESLPIEVMVDAVHKLKYENYTSSFFIRDIIKPDPPKNLQLKPLKNSRQVEVSWEYPD
TWSTPHSYFSLTFCVQVQGKSKREKKDRVFTDKTSATVICRKNASISVRAQDRYYSSSWSEWASVPCS
RAEFLENGGTSLSEKTVLLLVTPFLAAAWSLHPAGIDGSGATNFSLLKQAGDVEENPGPLEGTSLLCW
MALCLLGADHADAGIHVFILGCFSAGLPKTEANWVNVISDLKKIEDLIQSMHIDATLYTESDVHPSCK
VTAMKCFLLELQVISLESGDASIHDTVENLIILANNSLSSNGNVTESGCKECEELEEKNIKEFLQSFV
HIVQMFINTSRARAEFLENGGTSLSEKTVLLLVTPFLAAAWSLHPGSGEGRGSLLTCGDVEENPGPAL
PVTALLLPLALLLHAARPDIQMTOTTSSLSASLGDRVTISCRASQDISKYLNWYQQKPDGTVKLLIYH
TSRLHSGVPSRFSGSGSGTDYSLTISNLEQEDIATYFCQQGNTLPYTFGGGTKLEITGGGGSGGGGSG
GGGSEVKLQESGPGLVAPSQSLSVTCTVSGVSLPDYGVSWIRQPPRKGLEWLGVIWGSETTYYNSALK
SRLTIIKDNSKSQVFLKMNSLQTDDTAIYYCAKHYYYGGSYAMDYWGQGTSVTVSSTTTPAPRPPTPA
PTIASQPLSLRPEACRPAAGGAVHTRGLDFACDIYIWAPLAGTCGVLLLSLVITLYCKRGRKKLLYIF
KOPFMRPVQTTQEEDGCSCRFPEEEEGGCELRVKFSRSADAPAYQQGQNQLYNELNLGRREEYDVLDK
RRGRDPEMGGKPRRKNPQEGLYNELQKDKMAEAYSEIGMKGERRRGKGHDGLYQGLSTATKDTYDALH
MQALPPR (SEQ ID NO: 699)

TABLE A-8
NUCLEIC ACID SEQUENCES OF VIRAL VECTOR CONSTRUCTS WITH CAR
AND ONE CYTOKINE
General structure 1: SP-Cytokine-Anchoring structure-Linker-CAR
General structure 2: CAR-Linker-SP-Cytokine-Anchoring structure
General structure 3: CAR-Linker-SP-Cytokine Soluble form
Exemplary molecule 1 CAR19-T2A-SP-IL-4-Anchoring structure

| SEQ | | |
|---|---|---|
| ID | | |
| NO: | Sequence | Description |
| 510 | gccttaccagtgaccgccttgctcctgccgctggccttgctgctccacgc | CAR19-T2A- |
| | cgccaggccggacatccagatgacacagactacatcctccctgtctgcct | SP-IL-4- |
| | ctctgggagacagagtcaccatcagttgcagggcaagtcaggacattagt | Anchoring |
| | aaatatttaaattggtatcagcagaaaccagatggaactgttaaactcct | structure full |
| | gatctaccatacatcaagattacactcaggagtcccatcaaggttcagtg | construct |
| | gcagtgggtctggaacagattattctctcaccattagcaacctggagcaa | |
| | gaagatattgccacttacttttgccaacagggtaatacgcttccgtacac | |
| | gttcggaggggggaccaagctggagatcacaggtggcggtggctcgggcg | |
| | gtggtgggtcgggtggcggcggatctgaggtgaaactgcaggagtcagga | |
| | cctggcctggtggcgccctcacagagcctgtccgtcacatgcactgtctc | |
| | aggggtctcattacccgactatggtgtaagctggattcgccagcctccac | |
| | gaaagggtctggagtggctgggagtaatatggggtagtgaaaccacatac | |
| | tataattcagctctcaaatccagactgaccatcatcaaggacaactccaa | |
| | gagccaagttttcttaaaaatgaacagtctgcaaactgatgacacagcca | |
| | tttactactgtgccaaacattattactacggtggtagctatgctatggac | |
| | tactggggccaaggaacctcagtcaccgtctcctcaaccacgacgccagc | |
| | gccgcgaccaccaacaccggcgcccaccatcgcgtcgcagcccctgtccc | |
| | tgcgcccagaggcgtgccggccagcggcggggggcgcagtgcacacgagg | |
| | gggctggacttcgcctgtgatatctacatctgggcgcccttggccgggac | |
| | ttgtggggtccttctcctgtcactggttatcaccctttactgcaaacggg | |
| | gcagaaagaaactcctgtatatattcaaacaaccatttatgagaccagta | |
| | caaactactcaagaggaagatggctgtagctgccgatttccagaagaaga | |
| | agaaggaggatgtgaactgagagtgaagttcagcaggagcgcagacgccc | |
| | ccgcgtaccagcagggccagaaccagctctataacgagctcaatctagga | |
| | cgaagagaggagtacgatgttttggacaagagacgtggccgggaccctga | |
| | gatggggggaaagccgagaaggaagaaccctcaggaaggcctgtacaatg | |
| | aactgcagaaagataagatggcggaggcctacagtgagattgggatgaaa | |
| | ggcgagcgccggaggggcaaggggcacgatggcctttaccagggtctcag | |
| | tacagccaccaaggacacctacgacgcccttcacatgcaggccctgcccc | |
| | ctcgcggctccggcgagggcaggggaagtctactaacatgcggggacgtg | |
| | gaggaaaatcccggccccggtctcacctcccaactgcttccccctctgtt | |
| | cttcctgctagcatgtgccggcaactttgtccacggacacaagtgcgata | |
| | tcaccttacaggagatcatcaaaactttgaacagcctcacagagcagaag | |
| | actctgtgcaccgagttgaccgtaacagacatctttgctgcctccaagaa | |
| | cacaactgagaaggaaaccttctgcagggctgcgactgtgctccggcagt | |
| | tctacagccaccatgagaaggacactcgctgcctgggtgcgactgcacag | |
| | cagttccacaggcacaagcagctgatccgattcctgaaacggctcgacag | |
| | gaacctctggggcctggcgggcttgaattcctgtcctgtgaaggaagcca | |
| | accagagtacgttggaaaacttcttggaaaggctaaagacgatcatgaga | |
| | gagaaatattcaaagtgttcgagctctggaggaggaggatctggcggagg | |
| | aggaagtggaggaggaggctctggaggaggcggatctggaggagggagtc | |
| | tgcagcttgaaaatggtgggacatccttatcagagaaaacagttcttctg | |
| | ctggtgactccatttctggcagcagcctggagccttcatccc | |
| 511 | ggtctcacctcccaactgcttccccctctgttcttcctgctagcatgtgc | SP-IL-4- |
| | cggcaactttgtccacggacacaagtgcgatatcaccttacaggagatca | Anchoring |
| | tcaaaactttgaacagcctcacagagcagaagactctgtgcaccgagttg | structure |
| | accgtaacagacatctttgctgcctccaagaacacaactgagaaggaaac | |
| | cttctgcagggctgcgactgtgctccggcagttctacagccaccatgaga | |
| | aggacactcgctgcctgggtgcgactgcacagcagttccacaggcacaag | |
| | cagctgatccgattcctgaaacggctcgacaggaacctctggggcctggc | |
| | gggcttgaattcctgtcctgtgaaggaagccaaccagagtacgttggaaa | |
| | acttcttggaaaggctaaagacgatcatgagagagaaatattcaaagtgt | |
| | tcgagctctggaggaggaggatctggcggaggaggaagtggaggaggagg | |
| | ctctggaggaggcggatctggaggagggagtctgcagcttgaaaatggtg | |
| | ggacatccttatcagagaaaacagttcttctgctggtgactccatttctg | |
| | gcagcagcctggagccttcatccc | |
| 450 | gccttaccagtgaccgccttgctcctgccgctggccttgctgctccacgc | Leader (CD8α) |
| | cgccaggccg | |
| 451 | gacatccagatgacacagactacatcctccctgtctgcctctctgggaga | scFv (CD19) |
| | cagagtcaccatcagttgcagggcaagtcaggacattagtaaatatttaa | |
| | attggtatcagcagaaaccagatggaactgttaaactcctgatctaccat | |
| | acatcaagattacactcaggagtcccatcaaggttcagtggcagtgggtc | |
| | tggaacagattattctctcaccattagcaacctggagcaagaagatattg | |
| | ccacttacttttgccaacagggtaatacgcttccgtacacgttcggaggg | |
| | gggaccaagctggagatcacaggtggcggtggctcgggcggtggtgggtc | |
| | gggtggcggcggatctgaggtgaaactgcaggagtcaggacctggcctgg | |
| | tggcgccctcacagagcctgtccgtcacatgcactgtctcaggggtctca | |
| | ttacccgactatggtgtaagctggattcgccagcctccacgaaagggtct | |
| | ggagtggctgggagtaatatggggtagtgaaaccacatactataattcag | |
| | ctctcaaatccagactgaccatcatcaaggacaactccaagagccaagtt | |
| | ttcttaaaaatgaacagtctgcaaactgatgacacagccatttactactg | |
| | tgccaaacattattactacggtggtagctatgctatggactactggggcc | |
| | aaggaacctcagtcaccgtctcctca | |
| 452 | accacgacgccagcgccgcgaccaccaacaccggcgcccaccatcgcgtc | Hinge (CD8α) |
| | gcagcccctgtccctgcgcccagaggcgtgccggccagcggggggggcg | |
| | cagtgcacacgagggggctggacttcgcctgtgat | |
| 453 | atctacatctgggcgcccttggccgggacttgtggggtccttctcctgtc | Transmembrane |
| | actggttatcaccctttactgc | domain (CD8α) |
| 454 | aaacggggcagaaagaaactcctgtatatattcaaacaaccatttatgag | Costimulatory |
| | accagtacaaactactcaagaggaagatggctgtagctgccgatttccag | domain (4- |
| | aagaagaagaaggaggatgtgaactg | 1BB) |
| 455 | agagtgaagttcagcaggagcgcagacgcccccgcgtaccagcagggcca | Intracellular |
| | gaaccagctctataacgagctcaatctaggacgaagagaggagtacgatg | signal domain |
| | ttttggacaagagacgtggccgggaccctgagatggggggaaagccgaga | (CD3ζ) |
| | aggaagaaccctcaggaaggcctgtacaatgaactgcagaaagataagat | |
| | ggcggaggcctacagtgagattgggatgaaaggcgagcgccggaggggca | |
| | aggggcacgatggcctttaccagggtctcagtacagccaccaaggacacc | |
| | tacgacgcccttcacatgcaggccctgccccctcgc | |
| 456 | gccttaccagtgaccgccttgctcctgccgctggccttgctgctccacgc | Complete |
| | cgccaggccggacatccagatgacacagactacatcctccctgtctgcct | sequence of |
| | ctctgggagacagagtcaccatcagttgcagggcaagtcaggacattagt | CAR (CD8α |
| | aaatatttaaattggtatcagcagaaaccagatggaactgttaaactcct | Leader + Anti |
| | gatctaccatacatcaagattacactcaggagtcccatcaaggttcagtg | CD19- |
| | gcagtgggtctggaacagattattctctcaccattagcaacctggagcaa | scFv + CD8α |
| | gaagatattgccacttacttttgccaacagggtaatacgcttccgtacac | Hinge + CD8α |
| | gttcggaggggggaccaagctggagatcacaggtggcggtggctcgggcg | Transmembrane |
| | gtggtgggtcgggtggcggcggatctgaggtgaaactgcaggagtcagga | + 4-1BB + CD3ζ) |
| | cctggcctggtggcgccctcacagagcctgtccgtcacatgcactgtctc | |
| | aggggtctcattacccgactatggtgtaagctggattcgccagcctccac | |
| | gaaagggtctggagtggctgggagtaatatggggtagtgaaaccacatac | |
| | tataattcagctctcaaatccagactgaccatcatcaaggacaactccaa | |
| | gagccaagttttcttaaaaatgaacagtctgcaaactgatgacacagcca | |
| | tttactactgtgccaaacattattactacggtggtagctatgctatggac | |
| | tactggggccaaggaacctcagtcaccgtctcctcaaccacgacgccagc | |
| | gccgcgaccaccaacaccggcgcccaccatcgcgtcgcagcccctgtccc | |
| | tgcgcccagaggcgtgccggccagcggcggggggcgcagtgcacacgagg | |
| | gggctggacttcgcctgtgatatctacatctgggcgcccttggccgggac | |
| | ttgtggggtccttctcctgtcactggttatcaccctttactgcaaacggg | |
| | gcagaaagaaactcctgtatatattcaaacaaccatttatgagaccagta | |
| | caaactactcaagaggaagatggctgtagctgccgatttccagaagaaga | |
| | agaaggaggatgtgaactgagagtgaagttcagcaggagcgcagacgccc | |
| | ccgcgtaccagcagggccagaaccagctctataacgagctcaatctagga | |
| | cgaagagaggagtacgatgttttggacaagagacgtggccgggaccctga | |
| | gatggggggaaagccgagaaggaagaaccctcaggaaggcctgtacaatg | |
| | aactgcagaaagataagatggcggaggcctacagtgagattgggatgaaa | |
| | ggcgagcgccggaggggcaaggggcacgatggcctttaccagggtctcag | |
| | tacagccaccaaggacacctacgacgcccttcacatgcaggccctgcccc | |
| | ctcgc | |
| | ggctccggc | linker |
| 351 | gagggcaggggaagtctactaacatgcggggacgtggaggaaaatcccgg | T2A peptide |
| | cccc | |
| 58 | ggtctcacctcccaactgcttccccctctgttcttcctgctagcatgtgc | Signal (IL-4) |
| | cggcaactttgtccacgga | |
| 163 | cacaagtgcgatatcaccttacaggagatcatcaaaactttgaacagcct | IL-4 |
| | cacagagcagaagactctgtgcaccgagttgaccgtaacagacatctttg | |
| | ctgcctccaagaacacaactgagaaggaaaccttctgcagggctgcgact | |
| | gtgctccggcagttctacagccaccatgagaaggacactcgctgcctggg | |
| | tgcgactgcacagcagttccacaggcacaagcagctgatccgattcctga | |
| | aacggctcgacaggaacctctggggcctggcgggcttgaattcctgtcct | |
| | gtgaaggaagccaaccagagtacgttggaaaacttcttggaaaggctaaa | |
| | gacgatcatgagagagaaatattcaaagtgttcgagc | |
| 509 | tctggaggaggaggatctggcggaggaggaagtggaggaggaggctctgg | Lr1 |
| | aggaggcggatctggaggagggagtctgcag | |
| 250 | cttgaaaatggtgggacatccttatcagagaaaacagttcttctgctggt | GPI-attachment |
| | gactccatttctggcagcagcctggagccttcatccc | signal (CD59) |

Exemplary molecule 2
SP-IL-10-Anchoring structure-T2A-CAR19

| | | |
|---|---|---|
| 512 | cacagctcagcactgctctgttgcctggtcctcctgactggggtgagggc | SP-IL-10- |
| | cagcccaggccagggcacccagtctgagaacagctgcacccacttcccag | Anchoring |
| | gcaacctgcctaacatgcttcgagatctccgagatgccttcagcagagtg | structure-T2A- |
| | aagactttctttcaaatgaaggatcagctggacaacttgttgttaaagga | CAR19 |
| | gtccttgctggaggactttaagggttacctgggttgccaagccttgtctg | |
| | agatgatccagttttacctggaggaggtgatgccccaagctgagaaccaa | |
| | gacccagacatcaaggcgcatgtgaactccctgggggagaacctgaagac | |
| | cctcaggctgaggctacggcgctgtcatcgatttcttccctgtgaaaaca | |
| | agagcaaggccgtggagcaggtgaagaatgcctttaataagctccaagag | |
| | aaaggcatctacaaagccatgagtgagtttgacatcttcatcaactacat | |
| | agaagcctacatgacaatgaagatacgaaacTCTGGAGGAGGAGGATCTG | |
| | GCGGAGGAGGAAGTGGAGGAGGAGGCTCTGGAGGAGGCGGATCTGGAGGA | |
| | GGGAGTCTGCAGcttgaaaatggtgggacatccttatcagagaaaacagt | |
| | tcttctgctggtgactccatttctggcagcagcctggagccttcatcccg | |
| | gctccggcgagggcaggggaagtctactaacatgcggggacgtggaggaa | |
| | aatcccggccccgccttaccagtgaccgccttgctcctgccgctggcctt | |
| | gctgctccacgccgccaggccggacatccagatgacacagactacatcct | |
| | ccctgtctgcctctctgggagacagagtcaccatcagttgcagggcaagt | |
| | caggacattagtaaatatttaaattggtatcagcagaaaccagatggaac | |
| | tgttaaactcctgatctaccatacatcaagattacactcaggagtcccat | |
| | caaggttcagtggcagtgggtctggaacagattattctctcaccattagc | |
| | aacctggagcaagaagatattgccacttacttttgccaacagggtaatac | |
| | gcttccgtacacgttcggaggggggaccaagctggagatcacaggtggcg | |
| | gtggctcgggcggtggtgggtcgggtggcggcggatctgaggtgaaactg | |
| | caggagtcaggacctggcctggtggcgccctcacagagcctgtccgtcac | |
| | atgcactgtctcaggggtctcattacccgactatggtgtaagctggattc | |
| | gccagcctccacgaaagggtctggagtggctgggagtaatatggggtagt | |
| | gaaaccacatactataattcagctctcaaatccagactgaccatcatcaa | |
| | ggacaactccaagagccaagttttcttaaaaatgaacagtctgcaaactg | |
| | atgacacagccatttactactgtgccaaacattattactacggtggtagc | |
| | tatgctatggactactggggccaaggaacctcagtcaccgtctcctcaac | |
| | cacgacgccagcgccgcgaccaccaacaccggcgcccaccatcgcgtcgc | |
| | agcccctgtccctgcgcccagaggcgtgccggccagcggcggggggcgca | |
| | gtgcacacgagggggctggacttcgcctgtgatatctacatctgggcgcc | |
| | cttggccgggacttgtggggtccttctcctgtcactggttatcacccttt | |
| | actgcaaacggggcagaaagaaactcctgtatatattcaaacaaccattt | |
| | atgagaccagtacaaactactcaagaggaagatggctgtagctgccgatt | |
| | tccagaagaagaagaaggaggatgtgaactgagagtgaagttcagcagga | |
| | gcgcagacgcccccgcgtaccagcagggccagaaccagctctataacgag | |
| | ctcaatctaggacgaagagaggagtacgatgttttggacaagagacgtgg | |
| | ccgggaccctgagatggggggaaagccgagaaggaagaaccctcaggaag | |
| | gcctgtacaatgaactgcagaaagataagatggcggaggcctacagtgag | |
| | attgggatgaaaggcgagcgccggaggggcaaggggcacgatggccttta | |
| | ccagggtctcagtacagccaccaaggacacctacgacgcccttcacatgc | |
| | aggccctgccccctcgc | |
| 513 | cacagctcagcactgctctgttgcctggtcctcctgactggggtgagggc | SP-IL-10- |
| | cagcccaggccagggcacccagtctgagaacagctgcacccacttcccag | Anchoring |
| | gcaacctgcctaacatgcttcgagatctccgagatgccttcagcagagtg | structure |
| | aagactttctttcaaatgaaggatcagctggacaacttgttgttaaagga | |
| | gtccttgctggaggactttaagggttacctgggttgccaagccttgtctg | |
| | agatgatccagttttacctggaggaggtgatgccccaagctgagaaccaa | |
| | gacccagacatcaaggcgcatgtgaactccctgggggagaacctgaagac | |
| | cctcaggctgaggctacggcgctgtcatcgatttcttccctgtgaaaaca | |
| | agagcaaggccgtggagcaggtgaagaatgcctttaataagctccaagag | |
| | aaaggcatctacaaagccatgagtgagtttgacatcttcatcaactacat | |
| | agaagcctacatgacaatgaagatacgaaacTCTGGAGGAGGAGGATCTG | |
| | GCGGAGGAGGAAGTGGAGGAGGAGGCTCTGGAGGAGGCGGATCTGGAGGA | |
| | GGGAGTCTGCAGcttgaaaatggtgggacatccttatcagagaaaacagt | |
| | tcttctgctggtgactccatttctggcagcagcctggagccttcatcccg | |
| | gctccggc | |
| 59 | cacagctcagcactgctctgttgcctggtcctcctgactggggtgagggc | Signal (IL-10) |
| | c | |
| 164 | agcccaggccagggcacccagtctgagaacagctgcacccacttcccagg | IL-10 |
| | caacctgcctaacatgcttcgagatctccgagatgccttcagcagagtga | |
| | agactttctttcaaatgaaggatcagctggacaacttgttgttaaaggag | |
| | tccttgctggaggactttaagggttacctgggttgccaagccttgtctga | |
| | gatgatccagttttacctggaggaggtgatgccccaagctgagaaccaag | |
| | acccagacatcaaggcgcatgtgaactccctgggggagaacctgaagacc | |
| | ctcaggctgaggctacggcgctgtcatcgatttcttccctgtgaaaacaa | |
| | gagcaaggccgtggagcaggtgaagaatgcctttaataagctccaagaga | |
| | aaggcatctacaaagccatgagtgagtttgacatcttcatcaactacata | |
| | gaagcctacatgacaatgaagatacgaaac | |
| 509 | tctggaggaggaggatctggcggaggaggaagtggaggaggaggctctgg | Lr1 |
| | aggaggcggatctggaggagggagtctgcag | |
| 250 | cttgaaaatggtgggacatccttatcagagaaaacagttcttctgctggt | GPI-attachment |
| | gactccatttctggcagcagcctggagccttcatccc | signal (CD59) |
| | ggctccggc | linker |
| 351 | gagggcaggggaagtctactaacatgcggggacgtggaggaaaatcccgg | T2A peptide |
| | cccc | |
| | atg | Start codon |
| 450 | gccttaccagtgaccgccttgctcctgccgctggccttgctgctccacgc | Leader (CD8α) |
| | cgccaggccg | |
| 451 | gacatccagatgacacagactacatcctccctgtctgcctctctgggaga | scFv (CD19) |
| | cagagtcaccatcagttgcagggcaagtcaggacattagtaaatatttaa | |
| | attggtatcagcagaaaccagatggaactgttaaactcctgatctaccat | |
| | acatcaagattacactcaggagtcccatcaaggttcagtggcagtgggtc | |
| | tggaacagattattctctcaccattagcaacctggagcaagaagatattg | |
| | ccacttacttttgccaacagggtaatacgcttccgtacacgttcggaggg | |
| | gggaccaagctggagatcacaggtggcggtggctcgggcggtggtgggtc | |
| | gggtggcggcggatctgaggtgaaactgcaggagtcaggacctggcctgg | |
| | tggcgccctcacagagcctgtccgtcacatgcactgtctcaggggtctca | |
| | ttacccgactatggtgtaagctggattcgccagcctccacgaaagggtct | |
| | ggagtggctgggagtaatatggggtagtgaaaccacatactataattcag | |
| | ctctcaaatccagactgaccatcatcaaggacaactccaagagccaagtt | |
| | ttcttaaaaatgaacagtctgcaaactgatgacacagccatttactactg | |
| | tgccaaacattattactacggtggtagctatgctatggactactggggcc | |
| | aaggaacctcagtcaccgtctcctca | |
| 452 | accacgacgccagcgccgcgaccaccaacaccggcgcccaccatcgcgtc | Hinge (CD8α) |
| | gcagcccctgtccctgcgcccagaggcgtgccggccagcggcggggggcg | |
| | cagtgcacacgagggggctggacttcgcctgtgat | |
| 453 | atctacatctgggcgcccttggccgggacttgtggggtccttctcctgtc | Transmembrane |
| | actggttatcaccctttactgc | domain (CD8α) |
| 454 | aaacggggcagaaagaaactcctgtatatattcaaacaaccatttatgag | Costimulatory |
| | accagtacaaactactcaagaggaagatggctgtagctgccgatttccag | domain (4- |
| | aagaagaagaaggaggatgtgaactg | 1BB) |
| 455 | agagtgaagttcagcaggagcgcagacgcccccgcgtaccagcagggcca | Intracellular |
| | gaaccagctctataacgagctcaatctaggacgaagagaggagtacgatg | signal domain |
| | ttttggacaagagacgtggccgggaccctgagatggggggaaagccgaga | (CD3ζ) |
| | aggaagaaccctcaggaaggcctgtacaatgaactgcagaaagataagat | |
| | ggcggaggcctacagtgagattgggatgaaaggcgagcgccggaggggca | |
| | aggggcacgatggcctttaccagggtctcagtacagccaccaaggacacc | |
| | tacgacgcccttcacatgcaggccctgccccctcgc | |
| 456 | gccttaccagtgaccgccttgctcctgccgctggccttgctgctccacgc | Complete |
| | cgccaggccggacatccagatgacacagactacatcctccctgtctgcct | sequence of |
| | ctctgggagacagagtcaccatcagttgcagggcaagtcaggacattagt | CAR (CD8α |
| | aaatatttaaattggtatcagcagaaaccagatggaactgttaaactcct | Leader + Anti |
| | gatctaccatacatcaagattacactcaggagtcccatcaaggttcagtg | CD19- |
| | gcagtgggtctggaacagattattctctcaccattagcaacctggagcaa | scFv + CD8α |
| | gaagatattgccacttacttttgccaacagggtaatacgcttccgtacac | Hinge + CD8α |
| | gttcggaggggggaccaagctggagatcacaggtggcggtggctcgggcg | Transmembrane |
| | gtggtgggtcgggtggcggcggatctgaggtgaaactgcaggagtcagga | + 4-1BB + CD3) |
| | cctggcctggtggcgccctcacagagcctgtccgtcacatgcactgtctc | |
| | aggggtctcattacccgactatggtgtaagctggattcgccagcctccac | |
| | gaaagggtctggagtggctgggagtaatatggggtagtgaaaccacatac | |
| | tataattcagctctcaaatccagactgaccatcatcaaggacaactccaa | |
| | gagccaagttttcttaaaaatgaacagtctgcaaactgatgacacagcca | |
| | tttactactgtgccaaacattattactacggtggtagctatgctatggac | |
| | tactggggccaaggaacctcagtcaccgtctcctcaaccacgacgccagc | |
| | gccgcgaccaccaacaccggcgcccaccatcgcgtcgcagcccctgtccc | |
| | tgcgcccagaggcgtgccggccagcggcggggggcgcagtgcacacgagg | |
| | gggctggacttcgcctgtgatatctacatctgggcgcccttggccgggac | |
| | ttgtggggtccttctcctgtcactggttatcaccctttactgcaaacggg | |
| | gcagaaagaaactcctgtatatattcaaacaaccatttatgagaccagta | |
| | caaactactcaagaggaagatggctgtagctgccgatttccagaagaaga | |
| | agaaggaggatgtgaactgagagtgaagttcagcaggagcgcagacgccc | |
| | ccgcgtaccagcagggccagaaccagctctataacgagctcaatctagga | |
| | cgaagagaggagtacgatgttttggacaagagacgtggccgggaccctga | |
| | gatggggggaaagccgagaaggaagaaccctcaggaaggcctgtacaatg | |
| | aactgcagaaagataagatggcggaggcctacagtgagattgggatgaaa | |
| | ggcgagcgccggaggggcaaggggcacgatggcctttaccagggtctcag | |
| | tacagccaccaaggacacctacgacgcccttcacatgcaggccctgcccc | |
| | ctcgc | |

Exemplary molecule 3 CAR19-T2A-SP-IL-4-Lr1-Ar1

| | | |
|---|---|---|
| 518 | gccttaccagtgaccgccttgctcctgccgctggccttgctgctccacgc | CAR19-T2A- |
| | cgccaggccggacatccagatgacacagactacatcctccctgtctgcct | SP-IL-4-Lr1- |
| | ctctgggagacagagtcaccatcagttgcagggcaagtcaggacattagt | Ar1 full |
| | aaatatttaaattggtatcagcagaaaccagatggaactgttaaactcct | construct |
| | gatctaccatacatcaagattacactcaggagtcccatcaaggttcagtg | |
| | gcagtgggtctggaacagattattctctcaccattagcaacctggagcaa | |
| | gaagatattgccacttacttttgccaacagggtaatacgcttccgtacac | |
| | gttcggaggggggaccaagctggagatcacaggtggcggtggctcgggcg | |
| | gtggtgggtcgggtggcggcggatctgaggtgaaactgcaggagtcagga | |
| | cctggcctggtggcgccctcacagagcctgtccgtcacatgcactgtctc | |
| | aggggtctcattacccgactatggtgtaagctggattcgccagcctccac | |
| | gaaagggtctggagtggctgggagtaatatggggtagtgaaaccacatac | |
| | tataattcagctctcaaatccagactgaccatcatcaaggacaactccaa | |
| | gagccaagttttcttaaaaatgaacagtctgcaaactgatgacacagcca | |
| | tttactactgtgccaaacattattactacggtggtagctatgctatggac | |
| | tactggggccaaggaacctcagtcaccgtctcctcaaccacgacgccagc | |
| | gccgcgaccaccaacaccggcgcccaccatcgcgtcgcagcccctgtccc | |
| | tgcgcccagaggcgtgccggccagcggggggggcgcagtgcacacgagg | |
| | gggctggacttcgcctgtgatatctacatctgggcgcccttggccgggac | |
| | ttgtggggtccttctcctgtcactggttatcaccctttactgcaaacggg | |
| | gcagaaagaaactcctgtatatattcaaacaaccatttatgagaccagta | |
| | caaactactcaagaggaagatggctgtagctgccgatttccagaagaaga | |
| | agaaggaggatgtgaactgagagtgaagttcagcaggagcgcagacgccc | |
| | ccgcgtaccagcagggccagaaccagctctataacgagctcaatctagga | |
| | cgaagagaggagtacgatgttttggacaagagacgtggccgggaccctga | |
| | gatggggggaaagccgagaaggaagaaccctcaggaaggcctgtacaatg | |
| | aactgcagaaagataagatggcggaggcctacagtgagattgggatgaaa | |
| | ggcgagcgccggaggggcaaggggcacgatggcctttaccagggtctcag | |
| | tacagccaccaaggacacctacgacgcccttcacatgcaggccctgcccc | |
| | ctcgcggctccggcgagggcaggggaagtctactaacatgcggggacgtg | |
| | gaggaaaatcccggccccggtctcacctcccaactgcttccccctctgtt | |
| | cttcctgctagcatgtgccggcaactttgtccacggacacaagtgcgata | |
| | tcaccttacaggagatcatcaaaactttgaacagcctcacagagcagaag | |
| | actctgtgcaccgagttgaccgtaacagacatctttgctgcctccaagaa | |
| | cacaactgagaaggaaaccttctgcagggctgcgactgtgctccggcagt | |
| | tctacagccaccatgagaaggacactcgctgcctgggtgcgactgcacag | |
| | cagttccacaggcacaagcagctgatccgattcctgaaacggctcgacag | |
| | gaacctctggggcctggcgggcttgaattcctgtcctgtgaaggaagcca | |
| | accagagtacgttggaaaacttcttggaaaggctaaagacgatcatgaga | |
| | gagaaatattcaaagtgttcgagctctggaggaggaggatctggcggagg | |
| | aggaagtggaggaggaggctctggaggaggcggatctggaggagggagtc | |
| | tgcagatctacatctgggctcctctggctggcacctgcggagtgctgctg | |
| | ctgtctctggtgattact | |
| 450 | gccttaccagtgaccgccttgctcctgccgctggccttgctgctccacgc | Leader (CD8α) |
| | cgccaggccg | |
| 451 | gacatccagatgacacagactacatcctccctgtctgcctctctgggaga | scFv (CD19) |
| | cagagtcaccatcagttgcagggcaagtcaggacattagtaaatatttaa | |
| | attggtatcagcagaaaccagatggaactgttaaactcctgatctaccat | |
| | acatcaagattacactcaggagtcccatcaaggttcagtggcagtgggtc | |
| | tggaacagattattctctcaccattagcaacctggagcaagaagatattg | |
| | ccacttacttttgccaacagggtaatacgcttccgtacacgttcggaggg | |
| | gggaccaagctggagatcacaggtggcggtggctcgggcggtggtgggtc | |
| | gggtggcggcggatctgaggtgaaactgcaggagtcaggacctggcctgg | |
| | tggcgccctcacagagcctgtccgtcacatgcactgtctcaggggtctca | |
| | ttacccgactatggtgtaagctggattcgccagcctccacgaaagggtct | |
| | ggagtggctgggagtaatatggggtagtgaaaccacatactataattcag | |
| | ctctcaaatccagactgaccatcatcaaggacaactccaagagccaagtt | |
| | ttcttaaaaatgaacagtctgcaaactgatgacacagccatttactactg | |
| | tgccaaacattattactacggtggtagctatgctatggactactggggcc | |
| | aaggaacctcagtcaccgtctcctca | |
| 452 | accacgacgccagcgccgcgaccaccaacaccggcgcccaccatcgcgtc | Hinge (CD8α) |
| | gcagcccctgtccctgcgcccagaggcgtgccggccagcggcggggggcg | |
| | cagtgcacacgagggggctggacttcgcctgtgat | |
| 453 | atctacatctgggcgcccttggccgggacttgtggggtccttctcctgtc | Transmembrane |
| | actggttatcaccctttactgc | domain (CD8α) |
| 454 | aaacggggcagaaagaaactcctgtatatattcaaacaaccatttatgag | Costimulatory |
| | accagtacaaactactcaagaggaagatggctgtagctgccgatttccag | domain (4- |
| | aagaagaagaaggaggatgtgaactg | 1BB) |
| 455 | agagtgaagttcagcaggagcgcagacgcccccgcgtaccagcagggcca | Intracellular |
| | gaaccagctctataacgagctcaatctaggacgaagagaggagtacgatg | signal domain |
| | ttttggacaagagacgtggccgggaccctgagatggggggaaagccgaga | (CD3ζ) |
| | aggaagaaccctcaggaaggcctgtacaatgaactgcagaaagataagat | Complete |
| | ggcggaggcctacagtgagattgggatgaaaggcgagcgccggaggggca | sequence of |
| | aggggcacgatggcctttaccagggtctcagtacagccaccaaggacacc | CAR (CD8α |
| | tacgacgcccttcacatgcaggccctgccccctcgc | Leader + Anti |
| 456 | gccttaccagtgaccgccttgctcctgccgctggccttgctgctccacgc | CD19- |
| | cgccaggccggacatccagatgacacagactacatcctccctgtctgcct | scFv + CD8α |
| | ctctgggagacagagtcaccatcagttgcagggcaagtcaggacattagt | Hinge + CD8α |
| | aaatatttaaattggtatcagcagaaaccagatggaactgttaaactcct | Transmembrane |
| | gatctaccatacatcaagattacactcaggagtcccatcaaggttcagtg | + 4-1BB + CD3ζ) |
| | gcagtgggtctggaacagattattctctcaccattagcaacctggagcaa | |
| | gaagatattgccacttacttttgccaacagggtaatacgcttccgtacac | |
| | gttcggaggggggaccaagctggagatcacaggtggcggtggctcgggcg | |
| | gtggtgggtcgggtggcggcggatctgaggtgaaactgcaggagtcagga | |
| | cctggcctggtggcgccctcacagagcctgtccgtcacatgcactgtctc | |
| | aggggtctcattacccgactatggtgtaagctggattcgccagcctccac | |
| | gaaagggtctggagtggctgggagtaatatggggtagtgaaaccacatac | |
| | tataattcagctctcaaatccagactgaccatcatcaaggacaactccaa | |
| | gagccaagttttcttaaaaatgaacagtctgcaaactgatgacacagcca | |
| | tttactactgtgccaaacattattactacggtggtagctatgctatggac | |
| | tactggggccaaggaacctcagtcaccgtctcctcaaccacgacgccagc | |
| | gccgcgaccaccaacaccggcgcccaccatcgcgtcgcagcccctgtccc | |
| | tgcgcccagaggcgtgccggccagcggcggggggcgcagtgcacacgagg | |
| | gggctggacttcgcctgtgatatctacatctgggcgcccttggccgggac | |
| | ttgtggggtccttctcctgtcactggttatcaccctttactgcaaacggg | |
| | gcagaaagaaactcctgtatatattcaaacaaccatttatgagaccagta | |
| | caaactactcaagaggaagatggctgtagctgccgatttccagaagaaga | |
| | agaaggaggatgtgaactgagagtgaagttcagcaggagcgcagacgccc | |
| | ccgcgtaccagcagggccagaaccagctctataacgagctcaatctagga | |
| | cgaagagaggagtacgatgttttggacaagagacgtggccgggaccctga | |
| | gatggggggaaagccgagaaggaagaaccctcaggaaggcctgtacaatg | |
| | aactgcagaaagataagatggcggaggcctacagtgagattgggatgaaa | |
| | ggcgagcgccggaggggcaaggggcacgatggcctttaccagggtctcag | |
| | tacagccaccaaggacacctacgacgcccttcacatgcaggccctgcccc | |
| | ctcgc | |
| | ggctccggc | linker |
| 351 | gagggcaggggaagtctactaacatgcggggacgtggaggaaaatcccgg | T2A peptide |
| | cccc | |
| 58 | ggtctcacctcccaactgcttccccctctgttcttcctgctagcatgtgc | Signal (IL-4) |
| | cggcaactttgtccacgga | |
| 163 | cacaagtgcgatatcaccttacaggagatcatcaaaactttgaacagcct | IL-4 |
| | cacagagcagaagactctgtgcaccgagttgaccgtaacagacatctttg | |
| | ctgcctccaagaacacaactgagaaggaaaccttctgcagggctgcgact | |
| | gtgctccggcagttctacagccaccatgagaaggacactcgctgcctggg | |
| | tgcgactgcacagcagttccacaggcacaagcagctgatccgattcctga | |
| | aacggctcgacaggaacctctggggcctggcgggcttgaattcctgtcct | |
| | gtgaaggaagccaaccagagtacgttggaaaacttcttggaaaggctaaa | |
| | gacgatcatgagagagaaatattcaaagtgttcgagc | |
| 509 | tctggaggaggaggatctggcggaggaggaagtggaggaggaggctctgg | Lr1 |
| | aggaggcggatctggaggagggagtctgcag | |
| 253 | atctacatctgggctcctctggctggcacctgcggagtgctgctgctgtc | transmembrane |
| | tctggtgattact | peptide (Ar1) |

Exemplary molecule 4 CAR19-T2A-SP-IL-4-Lr1-Ar2

| | | |
|---|---|---|
| 519 | gccttaccagtgaccgccttgctcctgccgctggccttgctgctccacgc | CAR19-T2A- |
| | cgccaggccggacatccagatgacacagactacatcctccctgtctgcct | SP-IL-4-Lr1- |
| | ctctgggagacagagtcaccatcagttgcagggcaagtcaggacattagt | Ar2 Full |
| | aaatatttaaattggtatcagcagaaaccagatggaactgttaaactcct | construct |
| | gatctaccatacatcaagattacactcaggagtcccatcaaggttcagtg | |
| | gcagtgggtctggaacagattattctctcaccattagcaacctggagcaa | |
| | gaagatattgccacttacttttgccaacagggtaatacgcttccgtacac | |
| | gttcggaggggggaccaagctggagatcacaggtggcggtggctcgggcg | |
| | gtggtgggtcgggtggcggcggatctgaggtgaaactgcaggagtcagga | |
| | cctggcctggtggcgccctcacagagcctgtccgtcacatgcactgtctc | |
| | aggggtctcattacccgactatggtgtaagctggattcgccagcctccac | |
| | gaaagggtctggagtggctgggagtaatatggggtagtgaaaccacatac | |
| | tataattcagctctcaaatccagactgaccatcatcaaggacaactccaa | |
| | gagccaagttttcttaaaaatgaacagtctgcaaactgatgacacagcca | |
| | tttactactgtgccaaacattattactacggtggtagctatgctatggac | |
| | tactggggccaaggaacctcagtcaccgtctcctcaaccacgacgccagc | |
| | gccgcgaccaccaacaccggcgcccaccatcgcgtcgcagcccctgtccc | |
| | tgcgcccagaggcgtgccggccagcggcggggggcgcagtgcacacgagg | |
| | gggctggacttcgcctgtgatatctacatctgggcgcccttggccgggac | |
| | ttgtggggtccttctcctgtcactggttatcaccctttactgcaaacggg | |
| | gcagaaagaaactcctgtatatattcaaacaaccatttatgagaccagta | |
| | caaactactcaagaggaagatggctgtagctgccgatttccagaagaaga | |
| | agaaggaggatgtgaactgagagtgaagttcagcaggagcgcagacgccc | |
| | ccgcgtaccagcagggccagaaccagctctataacgagctcaatctagga | |
| | cgaagagaggagtacgatgttttggacaagagacgtggccgggaccctga | |
| | gatggggggaaagccgagaaggaagaaccctcaggaaggcctgtacaatg | |
| | aactgcagaaagataagatggcggaggcctacagtgagattgggatgaaa | |
| | ggcgagcgccggaggggcaaggggcacgatggcctttaccagggtctcag | |
| | tacagccaccaaggacacctacgacgcccttcacatgcaggccctgcccc | |
| | ctcgcggctccggcgagggcaggggaagtctactaacatgcggggacgtg | |
| | gaggaaaatcccggccccggtctcacctcccaactgcttccccctctgtt | |
| | cttcctgctagcatgtgccggcaactttgtccacggacacaagtgcgata | |
| | tcaccttacaggagatcatcaaaactttgaacagcctcacagagcagaag | |
| | actctgtgcaccgagttgaccgtaacagacatctttgctgcctccaagaa | |
| | cacaactgagaaggaaaccttctgcagggctgcgactgtgctccggcagt | |
| | tctacagccaccatgagaaggacactcgctgcctgggtgcgactgcacag | |
| | cagttccacaggcacaagcagctgatccgattcctgaaacggctcgacag | |
| | gaacctctggggcctggcgggcttgaattcctgtcctgtgaaggaagcca | |
| | accagagtacgttggaaaacttcttggaaaggctaaagacgatcatgaga | |
| | gagaaatattcaaagtgttcgagcagtggcgggggaggatctggaggcgg | |
| | aggatctgggggaggaggaagcggaggaggagggagcggaggaggcagcc | |
| | tgcagctgctgccttcttgggctatcaccctgatcagcgtgaacggaatc | |
| | tttgtgatctgttgtctgacttattgtttcgcccccagatgcagagagag | |
| | gagaaggaatgagagactgagaagagaatcagtgaggcccgtg | |
| 450 | gccttaccagtgaccgccttgctcctgccgctggccttgctgctccacgc | Leader (CD8α) |
| | cgccaggccg | |
| 451 | gacatccagatgacacagactacatcctccctgtctgcctctctgggaga | scFv (CD19) |
| | cagagtcaccatcagttgcagggcaagtcaggacattagtaaatatttaa | |
| | attggtatcagcagaaaccagatggaactgttaaactcctgatctaccat | |
| | acatcaagattacactcaggagtcccatcaaggttcagtggcagtgggtc | |
| | tggaacagattattctctcaccattagcaacctggagcaagaagatattg | |
| | ccacttacttttgccaacagggtaatacgcttccgtacacgttcggaggg | |
| | gggaccaagctggagatcacaggtggcggtggctcgggcggtggtgggtc | |
| | gggtggcggcggatctgaggtgaaactgcaggagtcaggacctggcctgg | |
| | tggcgccctcacagagcctgtccgtcacatgcactgtctcaggggtctca | |
| | ttacccgactatggtgtaagctggattcgccagcctccacgaaagggtct | |
| | ggagtggctgggagtaatatggggtagtgaaaccacatactataattcag | |
| | ctctcaaatccagactgaccatcatcaaggacaactccaagagccaagtt | |
| | ttcttaaaaatgaacagtctgcaaactgatgacacagccatttactactg | |
| | tgccaaacattattactacggtggtagctatgctatggactactggggcc | |
| | aaggaacctcagtcaccgtctcctca | |
| 452 | accacgacgccagcgccgcgaccaccaacaccggcgcccaccatcgcgtc | Hinge (CD8α) |
| | gcagcccctgtccctgcgcccagaggcgtgccggccagcggcggggggcg | |
| | cagtgcacacgagggggctggacttcgcctgtgat | |
| 453 | atctacatctgggcgcccttggccgggacttgtggggtccttctcctgtc | Transmembrane |
| | actggttatcaccctttactgc | domain (CD8α) |
| 454 | aaacggggcagaaagaaactcctgtatatattcaaacaaccatttatgag | Costimulatory |
| | accagtacaaactactcaagaggaagatggctgtagctgccgatttccag | domain (4- |
| | aagaagaagaaggaggatgtgaactg | 1BB) |
| 455 | agagtgaagttcagcaggagcgcagacgcccccgcgtaccagcagggcca | Intracellular |
| | gaaccagctctataacgagctcaatctaggacgaagagaggagtacgatg | signal domain |
| | ttttggacaagagacgtggccgggaccctgagatggggggaaagccgaga | (CD3ζ) |
| | aggaagaaccctcaggaaggcctgtacaatgaactgcagaaagataagat | Complete |
| | ggcggaggcctacagtgagattgggatgaaaggcgagcgccggaggggca | sequence of |
| | aggggcacgatggcctttaccagggtctcagtacagccaccaaggacacc | CAR (CD8α |
| | tacgacgcccttcacatgcaggccctgccccctcgc | Leader + Anti |
| 456 | gccttaccagtgaccgccttgctcctgccgctggccttgctgctccacgc | CD19- |
| | cgccaggccggacatccagatgacacagactacatcctccctgtctgcct | scFv + CD8α |
| | ctctgggagacagagtcaccatcagttgcagggcaagtcaggacattagt | Hinge + CD8α |
| | aaatatttaaattggtatcagcagaaaccagatggaactgttaaactcct | Transmembrane |
| | gatctaccatacatcaagattacactcaggagtcccatcaaggttcagtg | + 4-1BB + CD3ζ) |
| | gcagtgggtctggaacagattattctctcaccattagcaacctggagcaa | |
| | gaagatattgccacttacttttgccaacagggtaatacgcttccgtacac | |
| | gttcggaggggggaccaagctggagatcacaggtggcggtggctcgggcg | |
| | gtggtgggtcgggtggcggcggatctgaggtgaaactgcaggagtcagga | |
| | cctggcctggtggcgccctcacagagcctgtccgtcacatgcactgtctc | |
| | aggggtctcattacccgactatggtgtaagctggattcgccagcctccac | |
| | gaaagggtctggagtggctgggagtaatatggggtagtgaaaccacatac | |
| | tataattcagctctcaaatccagactgaccatcatcaaggacaactccaa | |
| | gagccaagttttcttaaaaatgaacagtctgcaaactgatgacacagcca | |
| | tttactactgtgccaaacattattactacggtggtagctatgctatggac | |
| | tactggggccaaggaacctcagtcaccgtctcctcaaccacgacgccagc | |
| | gccgcgaccaccaacaccggcgcccaccatcgcgtcgcagcccctgtccc | |
| | tgcgcccagaggcgtgccggccagcggggggggcgcagtgcacacgagg | |
| | gggctggacttcgcctgtgatatctacatctgggcgcccttggccgggac | |
| | ttgtggggtccttctcctgtcactggttatcaccctttactgcaaacggg | |
| | gcagaaagaaactcctgtatatattcaaacaaccatttatgagaccagta | |
| | caaactactcaagaggaagatggctgtagctgccgatttccagaagaaga | |
| | agaaggaggatgtgaactgagagtgaagttcagcaggagcgcagacgccc | |
| | ccgcgtaccagcagggccagaaccagctctataacgagctcaatctagga | |
| | cgaagagaggagtacgatgttttggacaagagacgtggccgggaccctga | |
| | gatggggggaaagccgagaaggaagaaccctcaggaaggcctgtacaatg | |
| | aactgcagaaagataagatggcggaggcctacagtgagattgggatgaaa | |
| | ggcgagcgccggaggggcaaggggcacgatggcctttaccagggtctcag | |
| | tacagccaccaaggacacctacgacgcccttcacatgcaggccctgcccc | |
| | ctcgc | |
| | ggctccggc | Peptide linker |
| 351 | gagggcaggggaagtctactaacatgcggggacgtggaggaaaatcccgg | T2A peptide |
| | cccc | |
| 58 | ggtctcacctcccaactgcttccccctctgttcttcctgctagcatgtgc | Signal (IL-4) |
| | cggcaactttgtccacgga | |
| 163 | cacaagtgcgatatcaccttacaggagatcatcaaaactttgaacagcct | IL-4 |
| | cacagagcagaagactctgtgcaccgagttgaccgtaacagacatctttg | |
| | ctgcctccaagaacacaactgagaaggaaaccttctgcagggctgcgact | |
| | gtgctccggcagttctacagccaccatgagaaggacactcgctgcctggg | |
| | tgcgactgcacagcagttccacaggcacaagcagctgatccgattcctga | |
| | aacggctcgacaggaacctctggggcctggcgggcttgaattcctgtcct | |
| | gtgaaggaagccaaccagagtacgttggaaaacttcttggaaaggctaaa | |
| | gacgatcatgagagagaaatattcaaagtgttcgagc | |
| 520 | agtggcgggggaggatctggaggcggaggatctgggggaggaggaagcgg | Lr1 |
| | aggaggagggagcggaggaggcagcctgcag | |
| 255 | ctgctgccttcttgggctatcaccctgatcagcgtgaacggaatctttgt | Transmembrane |
| | gatctgttgtctgacttattgtttcgcccccagatgcagagagaggagaa | peptide (Ar2) |
| | ggaatgagagactgagaagagaatcagtgaggcccgtg | |

Exemplary molecule 5 SP-IL-10-Lr1-Ar1-T2A-CAR19

| | | |
|---|---|---|
| 521 | cacagctcagcactgctctgttgcctggtcctcctgactggggtgagggc | SP-IL-10-Lr1- |
| | cagcccaggccagggcacccagtctgagaacagctgcacccacttcccag | Ar1-T2A- |
| | gcaacctgcctaacatgcttcgagatctccgagatgccttcagcagagtg | CAR19 Full |
| | aagactttctttcaaatgaaggatcagctggacaacttgttgttaaagga | construct |
| | gtccttgctggaggactttaagggttacctgggttgccaagccttgtctg | |
| | agatgatccagttttacctggaggaggtgatgccccaagctgagaaccaa | |
| | gacccagacatcaaggcgcatgtgaactccctgggggagaacctgaagac | |
| | cctcaggctgaggctacggcgctgtcatcgatttcttccctgtgaaaaca | |
| | agagcaaggccgtggagcaggtgaagaatgcctttaataagctccaagag | |
| | aaaggcatctacaaagccatgagtgagtttgacatcttcatcaactacat | |
| | agaagcctacatgacaatgaagatacgaaactctggaggaggaggatctg | |
| | gcggaggaggaagtggaggaggaggctctggaggaggcggatctggagga | |
| | gggagtctgcagatctacatctgggctcctctggctggcacctgcggagt | |
| | gctgctgctgtctctggtgattactggctccggcgagggcaggggaagtc | |
| | tactaacatgcggggacgtggaggaaaatcccggccccgccttaccagtg | |
| | accgccttgctcctgccgctggccttgctgctccacgccgccaggccgga | |
| | catccagatgacacagactacatcctccctgtctgcctctctgggagaca | |
| | gagtcaccatcagttgcagggcaagtcaggacattagtaaatatttaaat | |
| | tggtatcagcagaaaccagatggaactgttaaactcctgatctaccatac | |
| | atcaagattacactcaggagtcccatcaaggttcagtggcagtgggtctg | |
| | gaacagattattctctcaccattagcaacctggagcaagaagatattgcc | |
| | acttacttttgccaacagggtaatacgcttccgtacacgttcggaggggg | |
| | gaccaagctggagatcacaggtggcggtggctcgggcggtggtgggtcgg | |
| | gtggcggcggatctgaggtgaaactgcaggagtcaggacctggcctggtg | |
| | gcgccctcacagagcctgtccgtcacatgcactgtctcaggggtctcatt | |
| | acccgactatggtgtaagctggattcgccagcctccacgaaagggtctgg | |
| | agtggctgggagtaatatggggtagtgaaaccacatactataattcagct | |
| | ctcaaatccagactgaccatcatcaaggacaactccaagagccaagtttt | |
| | cttaaaaatgaacagtctgcaaactgatgacacagccatttactactgtg | |
| | ccaaacattattactacggtggtagctatgctatggactactggggccaa | |
| | ggaacctcagtcaccgtctcctcaaccacgacgccagcgccgcgaccacc | |
| | aacaccggcgcccaccatcgcgtcgcagcccctgtccctgcgcccagagg | |
| | cgtgccggccagcggcggggggcgcagtgcacacgagggggctggacttc | |
| | gcctgtgatatctacatctgggcgcccttggccgggacttgtggggtcct | |
| | tctcctgtcactggttatcaccctttactgcaaacggggcagaaagaaac | |
| | tcctgtatatattcaaacaaccatttatgagaccagtacaaactactcaa | |
| | gaggaagatggctgtagctgccgatttccagaagaagaagaaggaggatg | |
| | tgaactgagagtgaagttcagcaggagcgcagacgcccccgcgtaccagc | |
| | agggccagaaccagctctataacgagctcaatctaggacgaagagaggag | |
| | tacgatgttttggacaagagacgtggccgggaccctgagatggggggaaa | |
| | gccgagaaggaagaaccctcaggaaggcctgtacaatgaactgcagaaag | |
| | ataagatggcggaggcctacagtgagattgggatgaaaggcgagcgccgg | |
| | aggggcaaggggcacgatggcctttaccagggtctcagtacagccaccaa | |
| | ggacacctacgacgcccttcacatgcaggccctgccccctcgc | |
| 59 | cacagctcagcactgctctgttgcctggtcctcctgactggggtgagggc | Signal (IL-10) |
| | c | |
| 164 | agcccaggccagggcacccagtctgagaacagctgcacccacttcccagg | IL-10 |
| | caacctgcctaacatgcttcgagatctccgagatgccttcagcagagtga | |
| | agactttctttcaaatgaaggatcagctggacaacttgttgttaaaggag | |
| | tccttgctggaggactttaagggttacctgggttgccaagccttgtctga | |
| | gatgatccagttttacctggaggaggtgatgccccaagctgagaaccaag | |
| | acccagacatcaaggcgcatgtgaactccctgggggagaacctgaagacc | |
| | ctcaggctgaggctacggcgctgtcatcgatttcttccctgtgaaaacaa | |
| | gagcaaggccgtggagcaggtgaagaatgcctttaataagctccaagaga | |
| | aaggcatctacaaagccatgagtgagtttgacatcttcatcaactacata | |
| | gaagcctacatgacaatgaagatacgaaac | |
| 509 | tctggaggaggaggatctggcggaggaggaagtggaggaggaggctctgg | Lr1 |
| | aggaggcggatctggaggagggagtctgcag | |
| 253 | atctacatctgggctcctctggctggcacctgcggagtgctgctgctgtc | transmembrane |
| | tctggtgattact | peptide (Ar1) |
| | ggctccggc | linker |
| 351 | gagggcaggggaagtctactaacatgcggggacgtggaggaaaatcccgg | T2A peptide |
| | cccc | |
| 450 | gccttaccagtgaccgccttgctcctgccgctggccttgctgctccacgc | Leader (CD8α) |
| | cgccaggccg | |
| 451 | gacatccagatgacacagactacatcctccctgtctgcctctctgggaga | scFv (CD19) |
| | cagagtcaccatcagttgcagggcaagtcaggacattagtaaatatttaa | |
| | attggtatcagcagaaaccagatggaactgttaaactcctgatctaccat | |
| | acatcaagattacactcaggagtcccatcaaggttcagtggcagtgggtc | |
| | tggaacagattattctctcaccattagcaacctggagcaagaagatattg | |
| | ccacttacttttgccaacagggtaatacgcttccgtacacgttcggaggg | |
| | gggaccaagctggagatcacaggtggcggtggctcgggcggtggtgggtc | |
| | gggtggcggcggatctgaggtgaaactgcaggagtcaggacctggcctgg | |
| | tggcgccctcacagagcctgtccgtcacatgcactgtctcaggggtctca | |
| | ttacccgactatggtgtaagctggattcgccagcctccacgaaagggtct | |
| | ggagtggctgggagtaatatggggtagtgaaaccacatactataattcag | |
| | ctctcaaatccagactgaccatcatcaaggacaactccaagagccaagtt | |
| | ttcttaaaaatgaacagtctgcaaactgatgacacagccatttactactg | |
| | tgccaaacattattactacggtggtagctatgctatggactactggggcc | |
| | aaggaacctcagtcaccgtctcctca | |
| 452 | accacgacgccagcgccgcgaccaccaacaccggcgcccaccatcgcgtc | Hinge (CD8α) |
| | gcagcccctgtccctgcgcccagaggcgtgccggccagcggggggggcg | |
| | cagtgcacacgagggggctggacttcgcctgtgat | |
| 453 | atctacatctgggcgcccttggccgggacttgtggggtccttctcctgtc | Transmembrane |
| | actggttatcaccctttactgc | domain (CD8α) |
| 454 | aaacggggcagaaagaaactcctgtatatattcaaacaaccatttatgag | Costimulatory |
| | accagtacaaactactcaagaggaagatggctgtagctgccgatttccag | domain (4- |
| | aagaagaagaaggaggatgtgaactg | 1BB) |
| 455 | agagtgaagttcagcaggagcgcagacgcccccgcgtaccagcagggcca | Intracellular |
| | gaaccagctctataacgagctcaatctaggacgaagagaggagtacgatg | signal domain |
| | ttttggacaagagacgtggccgggaccctgagatggggggaaagccgaga | (CD3ζ) |
| | aggaagaaccctcaggaaggcctgtacaatgaactgcagaaagataagat | |
| | ggcggaggcctacagtgagattgggatgaaaggcgagcgccggaggggca | |
| | aggggcacgatggcctttaccagggtctcagtacagccaccaaggacacc | |
| | tacgacgcccttcacatgcaggccctgccccctcgc | |
| 456 | gccttaccagtgaccgccttgctcctgccgctggccttgctgctccacgc | Complete |
| | cgccaggccggacatccagatgacacagactacatcctccctgtctgcct | sequence of |
| | ctctgggagacagagtcaccatcagttgcagggcaagtcaggacattagt | CAR (CD8α |
| | aaatatttaaattggtatcagcagaaaccagatggaactgttaaactcct | Leader + Anti |
| | gatctaccatacatcaagattacactcaggagtcccatcaaggttcagtg | CD19- |
| | gcagtgggtctggaacagattattctctcaccattagcaacctggagcaa | scFv + CD8α |
| | gaagatattgccacttacttttgccaacagggtaatacgcttccgtacac | Hinge + CD8α |
| | gttcggaggggggaccaagctggagatcacaggtggcggtggctcgggcg | Transmembrane |
| | gtggtgggtcgggtggcggcggatctgaggtgaaactgcaggagtcagga | + 4-1BB + CD3ζ) |
| | cctggcctggtggcgccctcacagagcctgtccgtcacatgcactgtctc | |
| | aggggtctcattacccgactatggtgtaagctggattcgccagcctccac | |
| | gaaagggtctggagtggctgggagtaatatggggtagtgaaaccacatac | |
| | tataattcagctctcaaatccagactgaccatcatcaaggacaactccaa | |
| | gagccaagttttcttaaaaatgaacagtctgcaaactgatgacacagcca | |
| | tttactactgtgccaaacattattactacggtggtagctatgctatggac | |
| | tactggggccaaggaacctcagtcaccgtctcctcaaccacgacgccagc | |
| | gccgcgaccaccaacaccggcgcccaccatcgcgtcgcagcccctgtccc | |
| | tgcgcccagaggcgtgccggccagcggcggggggcgcagtgcacacgagg | |
| | gggctggacttcgcctgtgatatctacatctgggcgcccttggccgggac | |
| | ttgtggggtccttctcctgtcactggttatcaccctttactgcaaacggg | |
| | gcagaaagaaactcctgtatatattcaaacaaccatttatgagaccagta | |
| | caaactactcaagaggaagatggctgtagctgccgatttccagaagaaga | |
| | agaaggaggatgtgaactgagagtgaagttcagcaggagcgcagacgccc | |
| | ccgcgtaccagcagggccagaaccagctctataacgagctcaatctagga | |
| | cgaagagaggagtacgatgttttggacaagagacgtggccgggaccctga | |
| | gatggggggaaagccgagaaggaagaaccctcaggaaggcctgtacaatg | |
| | aactgcagaaagataagatggcggaggcctacagtgagattgggatgaaa | |
| | ggcgagcgccggaggggcaaggggcacgatggcctttaccagggtctcag | |
| | tacagccaccaaggacacctacgacgcccttcacatgcaggccctgcccc | |
| | ctcgc | |

Exemplary molecule 6 CAR19-T2A-SP-IL-10-Lr1-Ar1

| | | |
|---|---|---|
| 522 | gccttaccagtgaccgccttgctcctgccgctggccttgctgctccacgc | CAR19-T2A- |
| | cgccaggccggacatccagatgacacagactacatcctccctgtctgcct | SP-IL-10-Lr1- |
| | ctctgggagacagagtcaccatcagttgcagggcaagtcaggacattagt | Ar1 Full |
| | aaatatttaaattggtatcagcagaaaccagatggaactgttaaactcct | construct |
| | gatctaccatacatcaagattacactcaggagtcccatcaaggttcagtg | |
| | gcagtgggtctggaacagattattctctcaccattagcaacctggagcaa | |
| | gaagatattgccacttacttttgccaacagggtaatacgcttccgtacac | |
| | gttcggaggggggaccaagctggagatcacaggtggcggtggctcgggcg | |
| | gtggtgggtcgggtggcggcggatctgaggtgaaactgcaggagtcagga | |
| | cctggcctggtggcgccctcacagagcctgtccgtcacatgcactgtctc | |
| | aggggtctcattacccgactatggtgtaagctggattcgccagcctccac | |
| | gaaagggtctggagtggctgggagtaatatggggtagtgaaaccacatac | |
| | tataattcagctctcaaatccagactgaccatcatcaaggacaactccaa | |
| | gagccaagttttcttaaaaatgaacagtctgcaaactgatgacacagcca | |
| | tttactactgtgccaaacattattactacggtggtagctatgctatggac | |
| | tactggggccaaggaacctcagtcaccgtctcctcaaccacgacgccagc | |
| | gccgcgaccaccaacaccggcgcccaccatcgcgtcgcagcccctgtccc | |
| | tgcgcccagaggcgtgccggccagcggggggggcgcagtgcacacgagg | |
| | gggctggacttcgcctgtgatatctacatctgggcgcccttggccgggac | |
| | ttgtggggtccttctcctgtcactggttatcaccctttactgcaaacggg | |
| | gcagaaagaaactcctgtatatattcaaacaaccatttatgagaccagta | |
| | caaactactcaagaggaagatggctgtagctgccgatttccagaagaaga | |
| | agaaggaggatgtgaactgagagtgaagttcagcaggagcgcagacgccc | |
| | ccgcgtaccagcagggccagaaccagctctataacgagctcaatctagga | |
| | cgaagagaggagtacgatgttttggacaagagacgtggccgggaccctga | |
| | gatggggggaaagccgagaaggaagaaccctcaggaaggcctgtacaatg | |
| | aactgcagaaagataagatggcggaggcctacagtgagattgggatgaaa | |
| | ggcgagcgccggaggggcaaggggcacgatggcctttaccagggtctcag | |
| | tacagccaccaaggacacctacgacgcccttcacatgcaggccctgcccc | |
| | ctcgcggctccggcgagggcaggggaagtctactaacatgcggggacgtg | |
| | gaggaaaatcccggcccccacagctcagcactgctctgttgcctggtcct | |
| | cctgactggggtgagggccagcccaggccagggcacccagtctgagaaca | |
| | gctgcacccacttcccaggcaacctgcctaacatgcttcgagatctccga | |
| | gatgccttcagcagagtgaagactttctttcaaatgaaggatcagctgga | |
| | caacttgttgttaaaggagtccttgctggaggactttaagggttacctgg | |
| | gttgccaagccttgtctgagatgatccagttttacctggaggaggtgatg | |
| | ccccaagctgagaaccaagacccagacatcaaggcgcatgtgaactccct | |
| | gggggagaacctgaagaccctcaggctgaggctacggcgctgtcatcgat | |
| | ttcttccctgtgaaaacaagagcaaggccgtggagcaggtgaagaatgcc | |
| | tttaataagctccaagagaaaggcatctacaaagccatgagtgagtttga | |
| | catcttcatcaactacatagaagcctacatgacaatgaagatacgaaact | |
| | ctggaggaggaggatctggcggaggaggaagtggaggaggaggctctgga | |
| | ggaggcggatctggaggagggagtctgcagatctacatctgggctcctct | |
| | ggctggcacctgcggagtgctgctgctgtctctggtgattact | |
| 450 | gccttaccagtgaccgccttgctcctgccgctggccttgctgctccacgc | Leader (CD8α) |
| | cgccaggccg | |
| 451 | gacatccagatgacacagactacatcctccctgtctgcctctctgggaga | scFv (CD19) |
| | cagagtcaccatcagttgcagggcaagtcaggacattagtaaatatttaa | |
| | attggtatcagcagaaaccagatggaactgttaaactcctgatctaccat | |
| | acatcaagattacactcaggagtcccatcaaggttcagtggcagtgggtc | |
| | tggaacagattattctctcaccattagcaacctggagcaagaagatattg | |
| | ccacttacttttgccaacagggtaatacgcttccgtacacgttcggaggg | |
| | gggaccaagctggagatcacaggtggcggtggctcgggcggtggtgggtc | |
| | gggtggcggcggatctgaggtgaaactgcaggagtcaggacctggcctgg | |
| | tggcgccctcacagagcctgtccgtcacatgcactgtctcaggggtctca | |
| | ttacccgactatggtgtaagctggattcgccagcctccacgaaagggtct | |
| | ggagtggctgggagtaatatggggtagtgaaaccacatactataattcag | |
| | ctctcaaatccagactgaccatcatcaaggacaactccaagagccaagtt | |
| | ttcttaaaaatgaacagtctgcaaactgatgacacagccatttactactg | |
| | tgccaaacattattactacggtggtagctatgctatggactactggggcc | |
| | aaggaacctcagtcaccgtctcctca | |
| 452 | accacgacgccagcgccgcgaccaccaacaccggcgcccaccatcgcgtc | Hinge (CD8α) |
| | gcagcccctgtccctgcgcccagaggcgtgccggccagcggcggggggcg | |
| | cagtgcacacgagggggctggacttcgcctgtgat | |
| 453 | atctacatctgggcgcccttggccgggacttgtggggtccttctcctgtc | Transmembrane |
| | actggttatcaccctttactgc | domain (CD8α) |
| 454 | aaacggggcagaaagaaactcctgtatatattcaaacaaccatttatgag | Costimulatory |
| | accagtacaaactactcaagaggaagatggctgtagctgccgatttccag | domain (4- |
| | aagaagaagaaggaggatgtgaactg | 1BB) |
| 455 | agagtgaagttcagcaggagcgcagacgcccccgcgtaccagcagggcca | Intracellular |
| | gaaccagctctataacgagctcaatctaggacgaagagaggagtacgatg | signal domain |
| | ttttggacaagagacgtggccgggaccctgagatggggggaaagccgaga | (CD3ζ) |
| | aggaagaaccctcaggaaggcctgtacaatgaactgcagaaagataagat | |
| | ggcggaggcctacagtgagattgggatgaaaggcgagcgccggaggggca | |
| | aggggcacgatggcctttaccagggtctcagtacagccaccaaggacacc | |
| | tacgacgcccttcacatgcaggccctgccccctcgc | |
| 456 | gccttaccagtgaccgccttgctcctgccgctggccttgctgctccacgc | Complete |
| | cgccaggccggacatccagatgacacagactacatcctccctgtctgcct | sequence of |
| | ctctgggagacagagtcaccatcagttgcagggcaagtcaggacattagt | CAR (CD8α |
| | aaatatttaaattggtatcagcagaaaccagatggaactgttaaactcct | Leader + Anti |
| | gatctaccatacatcaagattacactcaggagtcccatcaaggttcagtg | CD19- |
| | gcagtgggtctggaacagattattctctcaccattagcaacctggagcaa | scFv + CD8α |
| | gaagatattgccacttacttttgccaacagggtaatacgcttccgtacac | Hinge + CD8α |
| | gttcggaggggggaccaagctggagatcacaggtggcggtggctcgggcg | |
| | gtggtgggtcgggtggcggcggatctgaggtgaaactgcaggagtcagga | Transmembrane |
| | cctggcctggtggcgccctcacagagcctgtccgtcacatgcactgtctc | + 4-1BB + CD3ζ) |
| | aggggtctcattacccgactatggtgtaagctggattcgccagcctccac | |
| | gaaagggtctggagtggctgggagtaatatggggtagtgaaaccacatac | |
| | tataattcagctctcaaatccagactgaccatcatcaaggacaactccaa | |
| | gagccaagttttcttaaaaatgaacagtctgcaaactgatgacacagcca | |
| | tttactactgtgccaaacattattactacggtggtagctatgctatggac | |
| | tactggggccaaggaacctcagtcaccgtctcctcaaccacgacgccagc | |
| | gccgcgaccaccaacaccggcgcccaccatcgcgtcgcagcccctgtccc | |
| | tgcgcccagaggcgtgccggccagcggcggggggcgcagtgcacacgagg | |
| | gggctggacttcgcctgtgatatctacatctgggcgcccttggccgggac | |
| | ttgtggggtccttctcctgtcactggttatcaccctttactgcaaacggg | |
| | gcagaaagaaactcctgtatatattcaaacaaccatttatgagaccagta | |
| | caaactactcaagaggaagatggctgtagctgccgatttccagaagaaga | |
| | agaaggaggatgtgaactgagagtgaagttcagcaggagcgcagacgccc | |
| | ccgcgtaccagcagggccagaaccagctctataacgagctcaatctagga | |
| | cgaagagaggagtacgatgttttggacaagagacgtggccgggaccctga | |
| | gatggggggaaagccgagaaggaagaaccctcaggaaggcctgtacaatg | |
| | aactgcagaaagataagatggcggaggcctacagtgagattgggatgaaa | |
| | ggcgagcgccggaggggcaaggggcacgatggcctttaccagggtctcag | |
| | tacagccaccaaggacacctacgacgcccttcacatgcaggccctgcccc | |
| | ctcgc | |
| | ggctccggc | Linker |
| 351 | gagggcaggggaagtctactaacatgcggggacgtggaggaaaatcccgg | T2A peptide |
| | cccc | |
| 59 | cacagctcagcactgctctgttgcctggtcctcctgactggggtgagggc | Signal (IL-10) |
| | c | |
| 164 | agcccaggccagggcacccagtctgagaacagctgcacccacttcccagg | IL-10 |
| | caacctgcctaacatgcttcgagatctccgagatgccttcagcagagtga | |
| | agactttctttcaaatgaaggatcagctggacaacttgttgttaaaggag | |
| | tccttgctggaggactttaagggttacctgggttgccaagccttgtctga | |
| | gatgatccagttttacctggaggaggtgatgccccaagctgagaaccaag | |
| | acccagacatcaaggcgcatgtgaactccctgggggagaacctgaagacc | |
| | ctcaggctgaggctacggcgctgtcatcgatttcttccctgtgaaaacaa | |
| | gagcaaggccgtggagcaggtgaagaatgcctttaataagctccaagaga | |
| | aaggcatctacaaagccatgagtgagtttgacatcttcatcaactacata | |
| | gaagcctacatgacaatgaagatacgaaac | |
| 509 | tctggaggaggaggatctggcggaggaggaagtggaggaggaggctctgg | Lr1 |
| | aggaggcggatctggaggagggagtctgcag | |
| 253 | atctacatctgggctcctctggctggcacctgcggagtgctgctgctgtc | transmembrane |
| | tctggtgattact | peptide (Ar1) |

Exemplary molecule 7
CAR19-T2A-SP-IL-10

| | | |
|---|---|---|
| 514 | gccttaccagtgaccgccttgctcctgccgctggccttgctgctccacgc | CAR19-T2A- |
| | cgccaggccggacatccagatgacacagactacatcctccctgtctgcct | SP-IL-10 |
| | ctctgggagacagagtcaccatcagttgcagggcaagtcaggacattagt | |
| | aaatatttaaattggtatcagcagaaaccagatggaactgttaaactcct | |
| | gatctaccatacatcaagattacactcaggagtcccatcaaggttcagtg | |
| | gcagtgggtctggaacagattattctctcaccattagcaacctggagcaa | |
| | gaagatattgccacttacttttgccaacagggtaatacgcttccgtacac | |
| | gttcggaggggggaccaagctggagatcacaggtggcggtggctcgggcg | |
| | gtggtgggtcgggtggcggcggatctgaggtgaaactgcaggagtcagga | |
| | cctggcctggtggcgccctcacagagcctgtccgtcacatgcactgtctc | |
| | aggggtctcattacccgactatggtgtaagctggattcgccagcctccac | |
| | gaaagggtctggagtggctgggagtaatatggggtagtgaaaccacatac | |
| | tataattcagctctcaaatccagactgaccatcatcaaggacaactccaa | |
| | gagccaagttttcttaaaaatgaacagtctgcaaactgatgacacagcca | |
| | tttactactgtgccaaacattattactacggtggtagctatgctatggac | |
| | tactggggccaaggaacctcagtcaccgtctcctcaaccacgacgccagc | |
| | gccgcgaccaccaacaccggcgcccaccatcgcgtcgcagcccctgtccc | |
| | tgcgcccagaggcgtgccggccagcggcggggggcgcagtgcacacgagg | |
| | gggctggacttcgcctgtgatatctacatctgggcgcccttggccgggac | |
| | ttgtggggtccttctcctgtcactggttatcaccctttactgcaaacggg | |
| | gcagaaagaaactcctgtatatattcaaacaaccatttatgagaccagta | |
| | caaactactcaagaggaagatggctgtagctgccgatttccagaagaaga | |
| | agaaggaggatgtgaactgagagtgaagttcagcaggagcgcagacgccc | |
| | ccgcgtaccagcagggccagaaccagctctataacgagctcaatctagga | |
| | cgaagagaggagtacgatgttttggacaagagacgtggccgggaccctga | |
| | gatggggggaaagccgagaaggaagaaccctcaggaaggcctgtacaatg | |
| | aactgcagaaagataagatggcggaggcctacagtgagattgggatgaaa | |
| | ggcgagcgccggaggggcaaggggcacgatggcctttaccagggtctcag | |
| | tacagccaccaaggacacctacgacgcccttcacatgcaggccctgcccc | |
| | ctcgcggctccggcgagggcaggggaagtctactaacatgcggggacgtg | |
| | gaggaaaatcccggcccccacagctcagcactgctctgttgcctggtcct | |
| | cctgactggggtgagggccagcccaggccagggcacccagtctgagaaca | |
| | gctgcacccacttcccaggcaacctgcctaacatgcttcgagatctccga | |
| | gatgccttcagcagagtgaagactttctttcaaatgaaggatcagctgga | |
| | caacttgttgttaaaggagtccttgctggaggactttaagggttacctgg | |
| | gttgccaagccttgtctgagatgatccagttttacctggaggaggtgatg | |
| | ccccaagctgagaaccaagacccagacatcaaggcgcatgtgaactccct | |
| | gggggagaacctgaagaccctcaggctgaggctacggcgctgtcatcgat | |
| | ttcttccctgtgaaaacaagagcaaggccgtggagcaggtgaagaatgcc | |
| | tttaataagctccaagagaaaggcatctacaaagccatgagtgagtttga | |
| | catcttcatcaactacatagaagcctacatgacaatgaagatacgaaac | |
| 450 | gccttaccagtgaccgccttgctcctgccgctggccttgctgctccacgc | Leader (CD8α) |
| | cgccaggccg | |
| 451 | gacatccagatgacacagactacatcctccctgtctgcctctctgggaga | scFv (CD19) |
| | cagagtcaccatcagttgcagggcaagtcaggacattagtaaatatttaa | |
| | attggtatcagcagaaaccagatggaactgttaaactcctgatctaccat | |
| | acatcaagattacactcaggagtcccatcaaggttcagtggcagtgggtc | |
| | tggaacagattattctctcaccattagcaacctggagcaagaagatattg | |
| | ccacttacttttgccaacagggtaatacgcttccgtacacgttcggaggg | |
| | gggaccaagctggagatcacaggtggcggtggctcgggcggtggtgggtc | |
| | gggtggcggcggatctgaggtgaaactgcaggagtcaggacctggcctgg | |
| | tggcgccctcacagagcctgtccgtcacatgcactgtctcaggggtctca | |
| | ttacccgactatggtgtaagctggattcgccagcctccacgaaagggtct | |
| | ggagtggctgggagtaatatggggtagtgaaaccacatactataattcag | |
| | ctctcaaatccagactgaccatcatcaaggacaactccaagagccaagtt | |
| | ttcttaaaaatgaacagtctgcaaactgatgacacagccatttactactg | |
| | tgccaaacattattactacggtggtagctatgctatggactactggggcc | |
| | aaggaacctcagtcaccgtctcctca | |
| 452 | accacgacgccagcgccgcgaccaccaacaccggcgcccaccatcgcgtc | Hinge (CD8α) |
| | gcagcccctgtccctgcgcccagaggcgtgccggccagcggcggggggcg | |
| | cagtgcacacgagggggctggacttcgcctgtgat | |
| 453 | atctacatctgggcgcccttggccgggacttgtggggtccttctcctgtc | Transmembrane |
| | actggttatcaccctttactgc | domain (CD8α) |
| 454 | aaacggggcagaaagaaactcctgtatatattcaaacaaccatttatgag | Costimulatory |
| | accagtacaaactactcaagaggaagatggctgtagctgccgatttccag | domain (4- |
| | aagaagaagaaggaggatgtgaactg | 1BB) |
| 455 | agagtgaagttcagcaggagcgcagacgcccccgcgtaccagcagggcca | Intracellular |
| | gaaccagctctataacgagctcaatctaggacgaagagaggagtacgatg | signal domain |
| | ttttggacaagagacgtggccgggaccctgagatggggggaaagccgaga | (CD3ζ) |
| | aggaagaaccctcaggaaggcctgtacaatgaactgcagaaagataagat | |
| | ggcggaggcctacagtgagattgggatgaaaggcgagcgccggaggggca | |
| 456 | aggggcacgatggcctttaccagggtctcagtacagccaccaaggacacc | |
| | tacgacgcccttcacatgcaggccctgccccctcgc | |
| | gccttaccagtgaccgccttgctcctgccgctggccttgctgctccacgc | Complete |
| | cgccaggccggacatccagatgacacagactacatcctccctgtctgcct | sequence of |
| | ctctgggagacagagtcaccatcagttgcagggcaagtcaggacattagt | CAR (CD8α |
| | aaatatttaaattggtatcagcagaaaccagatggaactgttaaactcct | Leader + Anti |
| | gatctaccatacatcaagattacactcaggagtcccatcaaggttcagtg | CD19- |
| | gcagtgggtctggaacagattattctctcaccattagcaacctggagcaa | scFv + CD8α |
| | gaagatattgccacttacttttgccaacagggtaatacgcttccgtacac | Hinge + CD8α |
| | gttcggaggggggaccaagctggagatcacaggtggcggtggctcgggcg | Transmembrane |
| | gtggtgggtcgggtggcggcggatctgaggtgaaactgcaggagtcagga | + 4-1BB + CD3ζ) |
| | cctggcctggtggcgccctcacagagcctgtccgtcacatgcactgtctc | |
| | aggggtctcattacccgactatggtgtaagctggattcgccagcctccac | |
| | gaaagggtctggagtggctgggagtaatatggggtagtgaaaccacatac | |
| | tataattcagctctcaaatccagactgaccatcatcaaggacaactccaa | |
| | gagccaagttttcttaaaaatgaacagtctgcaaactgatgacacagcca | |
| | tttactactgtgccaaacattattactacggtggtagctatgctatggac | |
| | tactggggccaaggaacctcagtcaccgtctcctcaaccacgacgccagc | |
| | gccgcgaccaccaacaccggcgcccaccatcgcgtcgcagcccctgtccc | |
| | tgcgcccagaggcgtgccggccagcggcggggggcgcagtgcacacgagg | |
| | gggctggacttcgcctgtgatatctacatctgggcgcccttggccgggac | |
| | ttgtggggtccttctcctgtcactggttatcaccctttactgcaaacggg | |
| | gcagaaagaaactcctgtatatattcaaacaaccatttatgagaccagta | |
| | caaactactcaagaggaagatggctgtagctgccgatttccagaagaaga | |
| | agaaggaggatgtgaactgagagtgaagttcagcaggagcgcagacgccc | |
| | ccgcgtaccagcagggccagaaccagctctataacgagctcaatctagga | |
| | cgaagagaggagtacgatgttttggacaagagacgtggccgggaccctga | |
| | gatggggggaaagccgagaaggaagaaccctcaggaaggcctgtacaatg | |
| | aactgcagaaagataagatggcggaggcctacagtgagattgggatgaaa | |
| | ggcgagcgccggaggggcaaggggcacgatggcctttaccagggtctcag | |
| | tacagccaccaaggacacctacgacgcccttcacatgcaggccctgcccc | |
| | ctcgc | |
| | ggctccggc | linker |
| 351 | gagggcaggggaagtctactaacatgcggggacgtggaggaaaatcccgg | T2A peptide |
| | cccc | |
| 59 | cacagctcagcactgctctgttgcctggtcctcctgactggggtgagggc | Signal (IL-10) |
| | c | |
| 164 | agcccaggccagggcacccagtctgagaacagctgcacccacttcccagg | IL-10 |
| | caacctgcctaacatgcttcgagatctccgagatgccttcagcagagtga | |
| | agactttctttcaaatgaaggatcagctggacaacttgttgttaaaggag | |
| | tccttgctggaggactttaagggttacctgggttgccaagccttgtctga | |
| | gatgatccagttttacctggaggaggtgatgccccaagctgagaaccaag | |
| | acccagacatcaaggcgcatgtgaactccctgggggagaacctgaagacc | |
| | ctcaggctgaggctacggcgctgtcatcgatttcttccctgtgaaaacaa | |
| | gagcaaggccgtggagcaggtgaagaatgcctttaataagctccaagaga | |
| | aaggcatctacaaagccatgagtgagtttgacatcttcatcaactacata | |
| | gaagcctacatgacaatgaagatacgaaac | |

TABLE A-9
AMINO ACID SEQUENCES OF VIRAL VECTOR CONSTRUCTS WITH CAR
AND ONE CYTOKINE

General structure 1: SP-Cytokine-Anchoring structure-Linker-CAR
General structure 2: CAR-Linker-SP-Cytokine-Anchoring structure
General structure 3: CAR-Linker-SP-Cytokine Soluble form
Exemplary molecule 1 CAR19-T2A-SP-IL-4-Anchoring structure

| SEQ | | |
|---|---|---|
| ID NO: | Sequence | Description |
| 515 | ALPVTALLLPLALLLHAARPDIQMTQTTSSLSASLGDRVTISCRASQD | CAR19- |
| | ISKYLNWYQQKPDGTVKLLIYHTSRLHSGVPSRFSGSGSGTDYSLTIS | T2A-SP-IL- |
| | NLEQEDIATYFCQQGNTLPYTFGGGTKLEITGGGGSGGGGSGGGGSEV | 4- |
| | KLQESGPGLVAPSQSLSVTCTVSGVSLPDYGVSWIRQPPRKGLEWLGV | Anchoring |
| | IWGSETTYYNSALKSRLTIIKDNSKSQVFLKMNSLQTDDTAIYYCAKH | structure |
| | YYYGGSYAMDYWGQGTSVTVSSTTTPAPRPPTPAPTIASQPLSLRPEA | Full |
| | CRPAAGGAVHTRGLDFACDIYIWAPLAGTCGVLLLSLVITLYCKRGRK | construct |
| | KLLYIFKQPFMRPVQTTQEEDGCSCRFPEEEEGGCELRVKFSRSADAP | |
| | AYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQ | |
| | ALPPRGSGEGRGSLLTCGDVEENPGPGLTSQLLPPLFFLLACAGNFVH | |
| | GHKCDITLQEIIKTLNSLTEQKTLCTELTVTDIFAASKNTTEKETFCR | |
| | AATVLRQFYSHHEKDTRCLGATAQQFHRHKQLIRFLKRLDRNLWGLAG | |
| | LNSCPVKEANQSTLENFLERLKTIMREKYSKCSSSGGGGSGGGGSGGG | |
| | GSGGGGSGGGSLQLENGGTSLSEKTVLLLVTPFLAAAWSLHP | |
| 400 | ALPVTALLLPLALLLHAARP | Leader |
| | | (CD8α) |
| 401 | DIQMTQTTSSLSASLGDRVTISCRASQDISKYLNWYQQKPDGTVKLLI | scFv |
| | YHTSRLHSGVPSRFSGSGSGTDYSLTISNLEQEDIATYFCQQGNTLPY | (CD19) |
| | TFGGGTKLEITGGGGSGGGGSGGGGSEVKLQESGPGLVAPSQSLSVTC | |
| | TVSGVSLPDYGVSWIRQPPRKGLEWLGVIWGSETTYYNSALKSRLTII | |
| | KDNSKSQVFLKMNSLQTDDTAIYYCAKHYYYGGSYAMDYWGQGTSVTV | |
| | SS | |
| 402 | TTTPAPRPPTPAPTIASQPLSLRPEACRPAAGGAVHTRGLDFACD | Hinge |
| | | (CD8α) |
| 403 | IYIWAPLAGTCGVLLLSLVITLYC | Transmembrane |
| | | domain |
| | | (CD8α) |
| 404 | KRGRKKLLYIFKQPFMRPVQTTQEEDGCSCRFPEEEEGGCEL | Costimulatory |
| | | domain |
| | | (4-1BB) |
| 405 | RVKFSRSADAPAYQQGQNQLYNELNLGRREEYDVLDKRRGRDPEMGGK | Intracellular |
| | PRRKNPQEGLYNELQKDKMAEAYSEIGMKGERRRGKGHDGLYQGLSTA | signal |
| | TKDTYDALHMQALPPR | domain |
| | | (CD3ζ) |
| 406 | ALPVTALLLPLALLLHAARPDIQMTQTTSSLSASLGDRVTISCRASQD | Complete |
| | ISKYLNWYQQKPDGTVKLLIYHTSRLHSGVPSRFSGSGSGTDYSLTIS | sequence of |
| | NLEQEDIATYFCQQGNTLPYTFGGGTKLEITGGGGSGGGGSGGGGSEV | CAR |
| | KLQESGPGLVAPSQSLSVTCTVSGVSLPDYGVSWIRQPPRKGLEWLGV | (CD8α |
| | IWGSETTYYNSALKSRLTIIKDNSKSQVFLKMNSLQTDDTAIYYCAKH | Leader + |
| | YYYGGSYAMDYWGQGTSVTVSSTTTPAPRPPTPAPTIASQPLSLRPEA | Anti CD19- |
| | CRPAAGGAVHTRGLDFACDIYIWAPLAGTCGVLLLSLVITLYCKRGRK | scFv + |
| | KLLYIFKQPFMRPVQTTQEEDGCSCRFPEEEEGGCELRVKFSRSADAP | CD8α |
| | AYQQGQNQLYNELNLGRREEYDVLDKRRGRDPEMGGKPRRKNPQEGLY | Hinge + |
| | NELQKDKMAEAYSEIGMKGERRRGKGHDGLYQGLSTATKDTYDALHMQ | CD8α |
| | ALPPR | Transmembrane |
| | | + 4- |
| | | 1BB + |
| | | CD3ζ) |
| | GSG | linker |
| 301 | EGRGSLLTCGDVEENPGP | T2A |
| | | peptide |
| 113 | GLTSQLLPPLFFLLACAGNFVHG | Signal (IL- |
| | | 4) |
| 101 | HKCDITLQEIIKTLNSLTEQKTLCTELTVTDIFAASKNTTEKETFCRA | IL-4 |
| | ATVLRQFYSHHEKDTRCLGATAQQFHRHKQLIRFLKRLDRNLWGLAGL | |
| | NSCPVKEANQSTLENFLERLKTIMREKYSKCSS | |
| 506 | SGGGGSGGGGSGGGGSGGGGSGGGSLQ | Lr1 |
| 200 | LENGGTSLSEKTVLLLVTPFLAAAWSLHP | GPI- |
| | | attachment |
| | | signal |
| | | (CD59) |

Exemplary molecule 2
SP-IL-10-Anchoring structure-T2A-CAR19

| | | |
|---|---|---|
| 516 | HSSALLCCLVLLTGVRASPGQGTQSENSCTHFPGNLPNMLRDLRDAFS | SP-IL-10- |
| | RVKTFFQMKDQLDNLLLKESLLEDFKGYLGCQALSEMIQFYLEEVMPQ | Anchoring |
| | AENQDPDIKAHVNSLGENLKTLRLRLRRCHRFLPCENKSKAVEQVKNA | structure- |
| | FNKLQEKGIYKAMSEFDIFINYIEAYMTMKIRNSGGGGSGGGGSGGGG | T2A- |
| | SGGGGSGGGSLQLENGGTSLSEKTVLLLVTPFLAAAWSLHPGSGEGRG | CAR19 Full |
| | SLLTCGDVEENPGPALPVTALLLPLALLLHAARPDIQMTQTTSSLSAS | construct |
| | LGDRVTISCRASQDISKYLNWYQQKPDGTVKLLIYHTSRLHSGVPSRF | |
| | SGSGSGTDYSLTISNLEQEDIATYFCQQGNTLPYTFGGGTKLEITGGG | |
| | GSGGGGSGGGGSEVKLQESGPGLVAPSQSLSVTCTVSGVSLPDYGVSW | |
| | IRQPPRKGLEWLGVIWGSETTYYNSALKSRLTIIKDNSKSQVFLKMNS | |
| | LQTDDTAIYYCAKHYYYGGSYAMDYWGQGTSVTVSSTTTPAPRPPTPA | |
| | PTIASQPLSLRPEACRPAAGGAVHTRGLDFACDIYIWAPLAGTCGVLL | |
| | LSLVITLYCKRGRKKLLYIFKQPFMRPVQTTQEEDGCSCRFPEEEEGG | |
| | CELRVKFSRSADAPAYQQGQNQLYNELNLGRREEYDVLDKRRGRDPEM | |
| | GGKPRRKNPQEGLYNELQKDKMAEAYSEIGMKGERRRGKGHDGLYQGL | |
| | STATKDTYDALHMQALPPR | |
| 114 | HSSALLCCLVLLTGVRA | Signal (IL- |
| | | 10) |
| 104 | SPGQGTQSENSCTHFPGNLPNMLRDLRDAFSRVKTFFQMKDQLDNLLL | IL-10 |
| | KESLLEDFKGYLGCQALSEMIQFYLEEVMPQAENQDPDIKAHVNSLGE | |
| | NLKTLRLRLRRCHRFLPCENKSKAVEQVKNAFNKLQEKGIYKAMSEFD | |
| | IFINYIEAYMTMKIRN | |
| 506 | SGGGGSGGGGSGGGGSGGGGSGGGSLQ | Lr1 |
| 200 | LENGGTSLSEKTVLLLVTPFLAAAWSLHP | GPI- |
| | | attachment |
| | | signal |
| | | (CD59) |
| | GSG | linker |
| 301 | EGRGSLLTCGDVEENPGP | T2A |
| | | peptide |
| 400 | ALPVTALLLPLALLLHAARP | Leader |
| | | (CD8α) |
| 401 | DIQMTQTTSSLSASLGDRVTISCRASQDISKYLNWYQQKPDGTVKLLI | scFv |
| | YHTSRLHSGVPSRFSGSGSGTDYSLTISNLEQEDIATYFCQQGNTLPY | (CD19) |
| | TFGGGTKLEITGGGGSGGGGSGGGGSEVKLQESGPGLVAPSQSLSVTC | |
| | TVSGVSLPDYGVSWIRQPPRKGLEWLGVIWGSETTYYNSALKSRLTII | |
| | KDNSKSQVFLKMNSLQTDDTAIYYCAKHYYYGGSYAMDYWGQGTSVTV | |
| | SS | |
| 402 | TTTPAPRPPTPAPTIASQPLSLRPEACRPAAGGAVHTRGLDFACD | Hinge |
| | | (CD8α) |
| 403 | IYIWAPLAGTCGVLLLSLVITLYC | Transmembrane |
| | | domain |
| | | (CD8α) |
| 404 | KRGRKKLLYIFKQPFMRPVQTTQEEDGCSCRFPEEEEGGCEL | Costimulatory |
| | | domain |
| | | (4-1BB) |
| 405 | RVKFSRSADAPAYQQGQNQLYNELNLGRREEYDVLDKRRGRDPEMGGK | Intracellular |
| | PRRKNPQEGLYNELQKDKMAEAYSEIGMKGERRRGKGHDGLYQGLSTA | signal |
| | TKDTYDALHMQALPPR | domain |
| | | (CD32) |
| 406 | ALPVTALLLPLALLLHAARPDIQMTQTTSSLSASLGDRVTISCRASQD | Complete |
| | ISKYLNWYQQKPDGTVKLLIYHTSRLHSGVPSRFSGSGSGTDYSLTIS | sequence of |
| | NLEQEDIATYFCQQGNTLPYTFGGGTKLEITGGGGSGGGGSGGGGSEV | CAR |
| | KLQESGPGLVAPSQSLSVTCTVSGVSLPDYGVSWIRQPPRKGLEWLGV | (CD8α |
| | IWGSETTYYNSALKSRLTIIKDNSKSQVFLKMNSLQTDDTAIYYCAKH | Leader + |
| | YYYGGSYAMDYWGQGTSVTVSSTTTPAPRPPTPAPTIASQPLSLRPEA | Anti CD19- |
| | CRPAAGGAVHTRGLDFACDIYIWAPLAGTCGVLLLSLVITLYCKRGRK | scFv + |
| | KLLYIFKQPFMRPVQTTQEEDGCSCRFPEEEEGGCELRVKFSRSADAP | CD8α |
| | AYQQGQNQLYNELNLGRREEYDVLDKRRGRDPEMGGKPRRKNPQEGLY | Hinge + |
| | NELQKDKMAEAYSEIGMKGERRRGKGHDGLYQGLSTATKDTYDALHMQ | CD8α |
| | ALPPR | Transmembrane |
| | | + 4- |
| | | 1BB + |
| | | CD3ζ) |

Exemplary molecule 3 CAR19-T2A-SP-IL-4-Lr1-Ar1

| | | |
|---|---|---|
| 523 | ALPVTALLLPLALLLHAARPDIQMTQTTSSLSASLGDRVTISCRASQD | CAR19- |
| | ISKYLNWYQQKPDGTVKLLIYHTSRLHSGVPSRFSGSGSGTDYSLTIS | T2A-SP-IL- |
| | NLEQEDIATYFCQQGNTLPYTFGGGTKLEITGGGGSGGGGSGGGGSEV | 4-Lr1-Ar1 |
| | KLQESGPGLVAPSQSLSVTCTVSGVSLPDYGVSWIRQPPRKGLEWLGV | full |
| | IWGSETTYYNSALKSRLTIIKDNSKSQVFLKMNSLQTDDTAIYYCAKH | construct |
| | YYYGGSYAMDYWGQGTSVTVSSTTTPAPRPPTPAPTIASQPLSLRPEA | |
| | CRPAAGGAVHTRGLDFACDIYIWAPLAGTCGVLLLSLVITLYCKRGRK | |
| | KLLYIFKQPFMRPVQTTQEEDGCSCRFPEEEEGGCELRVKFSRSADAP | |
| | AYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQ | |
| | ALPPRGSGEGRGSLLTCGDVEENPGPGLTSQLLPPLFFLLACAGNFVH | |
| | GHKCDITLQEIIKTLNSLTEQKTLCTELTVTDIFAASKNTTEKETFCR | |
| | AATVLRQFYSHHEKDTRCLGATAQQFHRHKQLIRFLKRLDRNLWGLAG | |
| | LNSCPVKEANQSTLENFLERLKTIMREKYSKCSSSGGGGSGGGGSGGG | |
| | GSGGGGSGGGSLQIYIWAPLAGTCGVLLLSLVIT | |
| 400 | ALPVTALLLPLALLLHAARP | Leader |
| | | (CD8α) |
| 401 | DIQMTQTTSSLSASLGDRVTISCRASQDISKYLNWYQQKPDGTVKLLI | scFv |
| | YHTSRLHSGVPSRFSGSGSGTDYSLTISNLEQEDIATYFCQQGNTLPY | (CD19) |
| | TFGGGTKLEITGGGGSGGGGSGGGGSEVKLQESGPGLVAPSQSLSVTC | |
| | TVSGVSLPDYGVSWIRQPPRKGLEWLGVIWGSETTYYNSALKSRLTII | |
| | KDNSKSQVFLKMNSLQTDDTAIYYCAKHYYYGGSYAMDYWGQGTSVTV | |
| | SS | |
| 402 | TTTPAPRPPTPAPTIASQPLSLRPEACRPAAGGAVHTRGLDFACD | Hinge |
| | | (CD8α) |
| 403 | IYIWAPLAGTCGVLLLSLVITLYC | Transmembrane |
| | | domain |
| | | (CD8α) |
| 404 | KRGRKKLLYIFKQPFMRPVQTTQEEDGCSCRFPEEEEGGCEL | Costimulatory |
| | | domain |
| | | (4-1BB) |
| 405 | RVKFSRSADAPAYQQGQNQLYNELNLGRREEYDVLDKRRGRDPEMGGK | Intracellular |
| | PRRKNPQEGLYNELQKDKMAEAYSEIGMKGERRRGKGHDGLYQGLSTA | signal |
| | TKDTYDALHMQALPPR | domain |
| | | (CD3ζ) |
| 406 | ALPVTALLLPLALLLHAARPDIQMTQTTSSLSASLGDRVTISCRASQD | Complete |
| | ISKYLNWYQQKPDGTVKLLIYHTSRLHSGVPSRFSGSGSGTDYSLTIS | sequence of |
| | NLEQEDIATYFCQQGNTLPYTFGGGTKLEITGGGGSGGGGSGGGGSEV | CAR |
| | KLQESGPGLVAPSQSLSVTCTVSGVSLPDYGVSWIRQPPRKGLEWLGV | (CD8α |
| | IWGSETTYYNSALKSRLTIIKDNSKSQVFLKMNSLQTDDTAIYYCAKH | Leader + |
| | YYYGGSYAMDYWGQGTSVTVSSTTTPAPRPPTPAPTIASQPLSLRPEA | Anti CD19- |
| | CRPAAGGAVHTRGLDFACDIYIWAPLAGTCGVLLLSLVITLYCKRGRK | scFv + |
| | KLLYIFKQPFMRPVQTTQEEDGCSCRFPEEEEGGCELRVKFSRSADAP | CD8α |
| | AYQQGQNQLYNELNLGRREEYDVLDKRRGRDPEMGGKPRRKNPQEGLY | Hinge + |
| | NELQKDKMAEAYSEIGMKGERRRGKGHDGLYQGLSTATKDTYDALHMQ | CD8α |
| | ALPPR | Transmembrane |
| | | + 4- |
| | | 1BB + |
| | | CD3ζ) |
| | GSG | linker |
| 301 | EGRGSLLTCGDVEENPGP | T2A |
| | | peptide |
| 113 | GLTSQLLPPLFFLLACAGNFVHG | Signal (IL- |
| | | 4) |
| 101 | HKCDITLQEIIKTLNSLTEQKTLCTELTVTDIFAASKNTTEKETFCRA | IL-4 |
| | ATVLRQFYSHHEKDTRCLGATAQQFHRHKQLIRFLKRLDRNLWGLAGL | |
| | NSCPVKEANQSTLENFLERLKTIMREKYSKCSS | |
| 506 | SGGGGSGGGGSGGGGSGGGGSGGGSLQ | Lr1 |
| 203 | IYIWAPLAGTCGVLLLSLVIT | transmembrane |
| | | peptide |
| | | (Ar1) |

Exemplary molecule 4 CAR19-T2A-SP-IL-4-Lr1-Ar2

| | | |
|---|---|---|
| 524 | ALPVTALLLPLALLLHAARPDIQMTQTTSSLSASLGDRVTISCRASQD | CAR19- |
| | ISKYLNWYQQKPDGTVKLLIYHTSRLHSGVPSRFSGSGSGTDYSLTIS | T2A-SP-IL- |
| | NLEQEDIATYFCQQGNTLPYTFGGGTKLEITGGGGSGGGGSGGGGSEV | 4-Lr1-Ar2 |
| | KLQESGPGLVAPSQSLSVTCTVSGVSLPDYGVSWIRQPPRKGLEWLGV | Full |
| | IWGSETTYYNSALKSRLTIIKDNSKSQVFLKMNSLQTDDTAIYYCAKH | construct |
| | YYYGGSYAMDYWGQGTSVTVSSTTTPAPRPPTPAPTIASQPLSLRPEA | |
| | CRPAAGGAVHTRGLDFACDIYIWAPLAGTCGVLLLSLVITLYCKRGRK | |
| | KLLYIFKQPFMRPVQTTQEEDGCSCRFPEEEEGGCELRVKFSRSADAP | |
| | AYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQ | |
| | ALPPRGSGEGRGSLLTCGDVEENPGPGLTSQLLPPLFFLLACAGNFVH | |
| | GHKCDITLQEIIKTLNSLTEQKTLCTELTVTDIFAASKNTTEKETFCR | |
| | AATVLRQFYSHHEKDTRCLGATAQQFHRHKQLIRFLKRLDRNLWGLAG | |
| | LNSCPVKEANQSTLENFLERLKTIMREKYSKCSSSGGGGSGGGGSGGG | |
| | GSGGGGSGGGSLQLLPSWAITLISVNGIFVICCLTYCFAPRCRERRRN | |
| | ERLRRESVRPV | |
| 400 | ALPVTALLLPLALLLHAARP | Leader |
| | | (CD8α) |
| 401 | DIQMTQTTSSLSASLGDRVTISCRASQDISKYLNWYQQKPDGTVKLLI | scFv |
| | YHTSRLHSGVPSRFSGSGSGTDYSLTISNLEQEDIATYFCQQGNTLPY | (CD19) |
| | TFGGGTKLEITGGGGSGGGGSGGGGSEVKLQESGPGLVAPSQSLSVTC | |
| | TVSGVSLPDYGVSWIRQPPRKGLEWLGVIWGSETTYYNSALKSRLTII | |
| | KDNSKSQVFLKMNSLQTDDTAIYYCAKHYYYGGSYAMDYWGQGTSVTV | |
| | SS | |
| 402 | TTTPAPRPPTPAPTIASQPLSLRPEACRPAAGGAVHTRGLDFACD | Hinge |
| | | (CD8α) |
| 403 | IYIWAPLAGTCGVLLLSLVITLYC | Transmembrane |
| | | domain |
| | | (CD8α) |
| 404 | KRGRKKLLYIFKQPFMRPVQTTQEEDGCSCRFPEEEEGGCEL | Costimulatory |
| | | domain |
| | | (4-1BB) |
| 405 | RVKFSRSADAPAYQQGQNQLYNELNLGRREEYDVLDKRRGRDPEMGGK | Intracellular |
| | PRRKNPQEGLYNELQKDKMAEAYSEIGMKGERRRGKGHDGLYQGLSTA | signal |
| | TKDTYDALHMQALPPR | domain |
| | | (CD3ζ) |
| 406 | ALPVTALLLPLALLLHAARPDIQMTQTTSSLSASLGDRVTISCRASQD | Complete |
| | ISKYLNWYQQKPDGTVKLLIYHTSRLHSGVPSRFSGSGSGTDYSLTIS | sequence of |
| | NLEQEDIATYFCQQGNTLPYTFGGGTKLEITGGGGSGGGGSGGGGSEV | CAR |
| | KLQESGPGLVAPSQSLSVTCTVSGVSLPDYGVSWIRQPPRKGLEWLGV | (CD8α |
| | IWGSETTYYNSALKSRLTIIKDNSKSQVFLKMNSLQTDDTAIYYCAKH | Leader + Anti |
| | YYYGGSYAMDYWGQGTSVTVSSTTTPAPRPPTPAPTIASQPLSLRPEA | CD19- |
| | CRPAAGGAVHTRGLDFACDIYIWAPLAGTCGVLLLSLVITLYCKRGRK | scFv + CD8α |
| | KLLYIFKQPFMRPVQTTQEEDGCSCRFPEEEEGGCELRVKFSRSADAP | Hinge + CD88 |
| | AYQQGQNQLYNELNLGRREEYDVLDKRRGRDPEMGGKPRRKNPQEGLY | Transmembrane |
| | NELQKDKMAEAYSEIGMKGERRRGKGHDGLYQGLSTATKDTYDALHMQ | + 4- |
| | ALPPR | 1BB + CD3ζ) |
| | GSG | Peptide |
| | | linker |
| 301 | EGRGSLLTCGDVEENPGP | T2A |
| | | peptide |
| 113 | GLTSQLLPPLFFLLACAGNFVHG | Signal (IL- |
| | | 4) |
| 101 | HKCDITLQEIIKTLNSLTEQKTLCTELTVTDIFAASKNTTEKETFCRA | IL-4 |
| | ATVLRQFYSHHEKDTRCLGATAQQFHRHKQLIRFLKRLDRNLWGLAGL | |
| | NSCPVKEANQSTLENFLERLKTIMREKYSKCSS | |
| 506 | SGGGGSGGGGSGGGGSGGGGSGGGSLQ | Lr1 |
| 204 | LLPSWAITLISVNGIFVICCLTYCFAPRCRERRRNERLRRESVRPV | Transmembrane |
| | | peptide |
| | | (Ar2) |

Exemplary molecule 5 SP-IL-10-Lr1-Ar1-T2A-CAR19

| | | |
|---|---|---|
| 525 | HSSALLCCLVLLTGVRASPGQGTQSENSCTHFPGNLPNMLRDLRDAFS | SP-IL-10- |
| | RVKTFFQMKDQLDNLLLKESLLEDFKGYLGCQALSEMIQFYLEEVMPQ | Lr1-Ar1- |
| | AENQDPDIKAHVNSLGENLKTLRLRLRRCHRFLPCENKSKAVEQVKNA | T2A- |
| | FNKLQEKGIYKAMSEFDIFINYIEAYMTMKIRNSGGGGSGGGGSGGGG | CAR19 Full |
| | SGGGGSGGGSLQIYIWAPLAGTCGVLLLSLVITGSGEGRGSLLTCGDV | construct |
| | EENPGPALPVTALLLPLALLLHAARPDIQMTQTTSSLSASLGDRVTIS | |
| | CRASQDISKYLNWYQQKPDGTVKLLIYHTSRLHSGVPSRFSGSGSGTD | |
| | YSLTISNLEQEDIATYFCQQGNTLPYTFGGGTKLEITGGGGSGGGGSG | |
| | GGGSEVKLQESGPGLVAPSQSLSVTCTVSGVSLPDYGVSWIRQPPRKG | |
| | LEWLGVIWGSETTYYNSALKSRLTIIKDNSKSQVFLKMNSLQTDDTAI | |
| | YYCAKHYYYGGSYAMDYWGQGTSVTVSSTTTPAPRPPTPAPTIASQPL | |
| | SLRPEACRPAAGGAVHTRGLDFACDIYIWAPLAGTCGVLLLSLVITLY | |
| | CKRGRKKLLYIFKQPFMRPVQTTQEEDGCSCRFPEEEEGGCELRVKES | |
| | RSADAPAYQQGQNQLYNELNLGRREEYDVLDKRRGRDPEMGGKPRRKN | |
| | PQEGLYNELQKDKMAEAYSEIGMKGERRRGKGHDGLYQGLSTATKDTY | |
| | DALHMQALPPR | |
| 114 | HSSALLCCLVLLTGVRA | Signal (IL- |
| | | 10) |
| 104 | SPGQGTQSENSCTHFPGNLPNMLRDLRDAFSRVKTFFQMKDQLDNLLL | IL-10 |
| | KESLLEDFKGYLGCQALSEMIQFYLEEVMPQAENQDPDIKAHVNSLGE | |
| | NLKTLRLRLRRCHRFLPCENKSKAVEQVKNAFNKLQEKGIYKAMSEFD | |
| | IFINYIEAYMTMKIRN | |
| 506 | SGGGGSGGGGSGGGGSGGGGSGGGSLQ | Lr1 |
| 203 | IYIWAPLAGTCGVLLLSLVIT | transmembrane |
| | | peptide |
| | | (Ar1) |
| | GSG | linker |
| 301 | EGRGSLLTCGDVEENPGP | T2A |
| | | peptide |
| 400 | ALPVTALLLPLALLLHAARP | Leader |
| | | (CD8α) |
| 401 | DIQMTQTTSSLSASLGDRVTISCRASQDISKYLNWYQQKPDGTVKLLI | scFv |
| | YHTSRLHSGVPSRFSGSGSGTDYSLTISNLEQEDIATYFCQQGNTLPY | (CD19) |
| | TFGGGTKLEITGGGGSGGGGSGGGGSEVKLQESGPGLVAPSQSLSVTC | |
| | TVSGVSLPDYGVSWIRQPPRKGLEWLGVIWGSETTYYNSALKSRLTII | |
| | KDNSKSQVFLKMNSLQTDDTAIYYCAKHYYYGGSYAMDYWGQGTSVTV | |
| | SS | |
| 402 | TTTPAPRPPTPAPTIASQPLSLRPEACRPAAGGAVHTRGLDFACD | Hinge |
| | | (CD8α) |
| 403 | IYIWAPLAGTCGVLLLSLVITLYC | Transmembrane |
| | | domain |
| | | (CD8α) |
| 404 | KRGRKKLLYIFKQPFMRPVQTTQEEDGCSCRFPEEEEGGCEL | Costimulatory |
| | | domain |
| | | (4-1BB) |
| 405 | RVKFSRSADAPAYQQGQNQLYNELNLGRREEYDVLDKRRGRDPEMGGK | Intracellular |
| | PRRKNPQEGLYNELQKDKMAEAYSEIGMKGERRRGKGHDGLYQGLSTA | signal |
| | TKDTYDALHMQALPPR | domain |
| | | (CD3ζ) |
| 406 | ALPVTALLLPLALLLHAARPDIQMTQTTSSLSASLGDRVTISCRASQD | Complete |
| | ISKYLNWYQQKPDGTVKLLIYHTSRLHSGVPSRFSGSGSGTDYSLTIS | sequence of |
| | NLEQEDIATYFCQQGNTLPYTFGGGTKLEITGGGGSGGGGSGGGGSEV | CAR |
| | KLQESGPGLVAPSQSLSVTCTVSGVSLPDYGVSWIRQPPRKGLEWLGV | (CD8α |
| | IWGSETTYYNSALKSRLTIIKDNSKSQVFLKMNSLQTDDTAIYYCAKH | Leader + Anti |
| | YYYGGSYAMDYWGQGTSVTVSSTTTPAPRPPTPAPTIASQPLSLRPEA | CD19- |
| | CRPAAGGAVHTRGLDFACDIYIWAPLAGTCGVLLLSLVITLYCKRGRK | scFv + CD8α |
| | KLLYIFKQPFMRPVQTTQEEDGCSCRFPEEEEGGCELRVKFSRSADAP | Hinge + CD88 |
| | AYQQGQNQLYNELNLGRREEYDVLDKRRGRDPEMGGKPRRKNPQEGLY | Transmembrane |
| | NELQKDKMAEAYSEIGMKGERRRGKGHDGLYQGLSTATKDTYDALHMQ | + 4- |
| | ALPPR | 1BB + CD3ζ) |

Exemplary molecule 6 CAR19-T2A-SP-IL-10-Lr1-Arl

| | | |
|---|---|---|
| 526 | ALPVTALLLPLALLLHAARPDIQMTQTTSSLSASLGDRVTISCRASQD | CAR19- |
| | ISKYLNWYQQKPDGTVKLLIYHTSRLHSGVPSRESGSGSGTDYSLTIS | T2A-SP-IL- |
| | NLEQEDIATYFCQQGNTLPYTFGGGTKLEITGGGGSGGGGSGGGGSEV | 10-Lr1-Ar1 |
| | KLQESGPGLVAPSQSLSVTCTVSGVSLPDYGVSWIRQPPRKGLEWLGV | Full |
| | IWGSETTYYNSALKSRLTIIKDNSKSQVFLKMNSLQTDDTAIYYCAKH | construct |
| | YYYGGSYAMDYWGQGTSVTVSSTTTPAPRPPTPAPTIASQPLSLRPEA | |
| | CRPAAGGAVHTRGLDFACDIYIWAPLAGTCGVLLLSLVITLYCKRGRK | |
| | KLLYIFKQPFMRPVQTTQEEDGCSCRFPEEEEGGCELRVKFSRSADAP | |
| | AYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQ | |
| | ALPPRGSGEGRGSLLTCGDVEENPGPHSSALLCCLVLLTGVRASPGQG | |
| | TQSENSCTHFPGNLPNMLRDLRDAFSRVKTFFQMKDQLDNLLLKESLL | |
| | EDFKGYLGCQALSEMIQFYLEEVMPQAENQDPDIKAHVNSLGENLKTL | |
| | RLRLRRCHRFLPCENKSKAVEQVKNAFNKLQEKGIYKAMSEFDIFINY | |
| | IEAYMTMKIRNSGGGGSGGGGSGGGGSGGGGSGGGSLQIYIWAPLAGT | |
| | CGVLLLSLVIT | |
| 400 | ALPVTALLLPLALLLHAARP | Leader |
| | | (CD8α) |
| 401 | DIQMTQTTSSLSASLGDRVTISCRASQDISKYLNWYQQKPDGTVKLLI | scFv |
| | YHTSRLHSGVPSRFSGSGSGTDYSLTISNLEQEDIATYFCQQGNTLPY | (CD19) |
| | TFGGGTKLEITGGGGSGGGGSGGGGSEVKLQESGPGLVAPSQSLSVTC | |
| | TVSGVSLPDYGVSWIRQPPRKGLEWLGVIWGSETTYYNSALKSRLTII | |
| | KDNSKSQVFLKMNSLQTDDTAIYYCAKHYYYGGSYAMDYWGQGTSVTV | |
| | SS | |
| 402 | TTTPAPRPPTPAPTIASQPLSLRPEACRPAAGGAVHTRGLDFACD | Hinge |
| | | (CD8α) |
| 403 | IYIWAPLAGTCGVLLLSLVITLYC | Transmembrane |
| | | domain |
| | | (CD8α) |
| 404 | KRGRKKLLYI FKQPFMRPVQTTQEEDGCSCRFPEEEEGGCEL | Costimulatory |
| | | domain |
| | | (4-1BB) |
| 405 | RVKFSRSADAPAYQQGQNQLYNELNLGRREEYDVLDKRRGRDPEMGGK | Intracellular |
| | PRRKNPQEGLYNELQKDKMAEAYSEIGMKGERRRGKGHDGLYQGLSTA | signal |
| | TKDTYDALHMQALPPR | domain |
| | | (CD3ζ) |
| 406 | ALPVTALLLPLALLLHAARPDIQMTQTTSSLSASLGDRVTISCRASQD | Complete |
| | ISKYLNWYQQKPDGTVKLLIYHTSRLHSGVPSRFSGSGSGTDYSLTIS | sequence of |
| | NLEQEDIATYFCQQGNTLPYTFGGGTKLEITGGGGSGGGGSGGGGSEV | CAR |
| | KLQESGPGLVAPSQSLSVTCTVSGVSLPDYGVSWIRQPPRKGLEWLGV | (CD8α |
| | IWGSETTYYNSALKSRLTIIKDNSKSQVFLKMNSLQTDDTAIYYCAKH | Leader + Anti |
| | YYYGGSYAMDYWGQGTSVTVSSTTTPAPRPPTPAPTIASQPLSLRPEA | CD19- |
| | CRPAAGGAVHTRGLDFACDIYIWAPLAGTCGVLLLSLVITLYCKRGRK | scFv + CD8α |
| | KLLYIFKQPFMRPVQTTQEEDGCSCRFPEEEEGGCELRVKFSRSADAP | Hinge + CD88 |
| | AYQQGQNQLYNELNLGRREEYDVLDKRRGRDPEMGGKPRRKNPQEGLY | Transmembrane |
| | NELQKDKMAEAYSEIGMKGERRRGKGHDGLYQGLSTATKDTYDALHMQ | + 4- |
| | ALPPR | 1BB + CD3ζ) |
| | GSG | Linker |
| 301 | EGRGSLLTCGDVEENPGP | T2A |
| | | peptide |
| 114 | HSSALLCCLVLLTGVRA | Signal (IL- |
| | | 10) |
| 104 | SPGQGTQSENSCTHFPGNLPNMLRDLRDAFSRVKTFFQMKDQLDNLLL | IL-10 |
| | KESLLEDFKGYLGCQALSEMIQFYLEEVMPQAENQDPDIKAHVNSLGE | |
| | NLKTLRLRLRRCHRFLPCENKSKAVEQVKNAFNKLQEKGIYKAMSEFD | |
| | IFINYIEAYMTMKIRN | |
| 506 | SGGGGSGGGGSGGGGSGGGGSGGGSLQ | Lr1 |
| 203 | IYIWAPLAGTCGVLLLSLVIT | Ar1 |

Exemplary molecule 7
CAR19-T2A-SP-IL-10

| | | |
|---|---|---|
| 517 | ALPVTALLLPLALLLHAARPDIQMTQTTSSLSASLGDRVTISCRASQD | Full |
| | ISKYLNWYQQKPDGTVKLLIYHTSRLHSGVPSRFSGSGSGTDYSLTIS | construct |
| | NLEQEDIATYFCQQGNTLPYTFGGGTKLEITGGGGSGGGGSGGGGSEV | |
| | KLQESGPGLVAPSQSLSVTCTVSGVSLPDYGVSWIRQPPRKGLEWLGV | |
| | IWGSETTYYNSALKSRLTIIKDNSKSQVFLKMNSLQTDDTAIYYCAKH | |
| | YYYGGSYAMDYWGQGTSVTVSSTTTPAPRPPTPAPTIASQPLSLRPEA | |
| | CRPAAGGAVHTRGLDFACDIYIWAPLAGTCGVLLLSLVITLYCKRGRK | |
| | KLLYIFKQPFMRPVQTTQEEDGCSCRFPEEEEGGCELRVKFSRSADAP | |
| | AYQQGQNQLYNELNLGRREEYDVLDKRRGRDPEMGGKPRRKNPQEGLY | |
| | NELQKDKMAEAYSEIGMKGERRRGKGHDGLYQGLSTATKDTYDALHMQ | |
| | ALPPRGSGEGRGSLLTCGDVEENPGPHSSALLCCLVLLTGVRASPGQG | |
| | TQSENSCTHFPGNLPNMLRDLRDAFSRVKTFFQMKDQLDNLLLKESLL | |
| | EDFKGYLGCQALSEMIQFYLEEVMPQAENQDPDIKAHVNSLGENLKTL | |
| | RLRLRRCHRFLPCENKSKAVEQVKNAFNKLQEKGIYKAMSEFDIFINY | |
| | IEAYMTMKIRN | |
| 400 | ALPVTALLLPLALLLHAARP | Leader |
| | | (CD8α) |
| 401 | DIQMTQTTSSLSASLGDRVTISCRASQDISKYLNWYQQKPDGTVKLLI | scFv |
| | YHTSRLHSGVPSRFSGSGSGTDYSLTISNLEQEDIATYFCQQGNTLPY | (CD19) |
| | TFGGGTKLEITGGGGSGGGGSGGGGSEVKLQESGPGLVAPSQSLSVTC | |
| | TVSGVSLPDYGVSWIRQPPRKGLEWLGVIWGSETTYYNSALKSRLTII | |
| | KDNSKSQVFLKMNSLQTDDTAIYYCAKHYYYGGSYAMDYWGQGTSVTV | |
| | SS | |
| 402 | TTTPAPRPPTPAPTIASQPLSLRPEACRPAAGGAVHTRGLDFACD | Hinge |
| | | (CD8α) |
| 403 | IYIWAPLAGTCGVLLLSLVITLYC | Transmembrane |
| | | domain |
| | | (CD8α) |
| 404 | KRGRKKLLYIFKQPFMRPVQTTQEEDGCSCRFPEEEEGGCEL | Costimulatory |
| | | domain |
| | | (4-1BB) |
| 405 | RVKFSRSADAPAYQQGQNQLYNELNLGRREEYDVLDKRRGRDPEMGGK | Intracellular |
| | PRRKNPQEGLYNELQKDKMAEAYSEIGMKGERRRGKGHDGLYQGLSTA | signal |
| | TKDTYDALHMQALPPR | domain |
| | | (CD3ζ) |
| 406 | ALPVTALLLPLALLLHAARPDIQMTQTTSSLSASLGDRVTISCRASQD | Complete |
| | ISKYLNWYQQKPDGTVKLLIYHTSRLHSGVPSRFSGSGSGTDYSLTIS | sequence of |
| | NLEQEDIATYFCQQGNTLPYTFGGGTKLEITGGGGSGGGGSGGGGSEV | CAR |
| | KLQESGPGLVAPSQSLSVTCTVSGVSLPDYGVSWIRQPPRKGLEWLGV | (CD8α |
| | IWGSETTYYNSALKSRLTIIKDNSKSQVFLKMNSLQTDDTAIYYCAKH | Leader + |
| | YYYGGSYAMDYWGQGTSVTVSSTTTPAPRPPTPAPTIASQPLSLRPEA | Anti CD19- |
| | CRPAAGGAVHTRGLDFACDIYIWAPLAGTCGVLLLSLVITLYCKRGRK | scFv + |
| | KLLYIFKQPFMRPVQTTQEEDGCSCRFPEEEEGGCELRVKFSRSADAP | CD8α |
| | AYQQGQNQLYNELNLGRREEYDVLDKRRGRDPEMGGKPRRKNPQEGLY | Hinge + |
| | NELQKDKMAEAYSEIGMKGERRRGKGHDGLYQGLSTATKDTYDALHMQ | CD8α |
| | ALPPR | Transmembrane |
| | | + 4- |
| | | 1BB + |
| | | CD3ζ) |
| | GSG | linker |
| 301 | EGRGSLLTCGDVEENPGP | T2A |
| | | peptide |
| 114 | HSSALLCCLVLLTGVRA | Signal (IL- |
| | | 10) |
| 104 | SPGQGTQSENSCTHFPGNLPNMLRDLRDAFSRVKTFFQMKDQLDNLLL | IL-10 |
| | KESLLEDFKGYLGCQALSEMIQFYLEEVMPQAENQDPDIKAHVNSLGE | |
| | NLKTLRLRLRRCHRFLPCENKSKAVEQVKNAFNKLQEKGIYKAMSEFD | |
| | IFINYIEAYMTMKIRN | |

TABLE A-10
NUCLEIC ACID SEQUENCES OF VIRAL VECTOR CONSTRUCTS WITH CAR
AND TWO CYTOKINES
Exemplary molecule 8 CAR19-T2A-SP-IL4-Anchoring structure-P2A-SP-IL15-Anchoring structure

| SEQ | | |
|---|---|---|
| ID NO: | Sequence | Description |
| 527 | gccttaccagtgaccgccttgctcctgccgctggccttgctgctccacgc | CAR19-T2A- |
| | cgccaggccggacatccagatgacacagactacatcctccctgtctgcct | SP-IL4- |
| | ctctgggagacagagtcaccatcagttgcagggcaagtcaggacattagt | Anchoring |
| | aaatatttaaattggtatcagcagaaaccagatggaactgttaaactcct | structure- |
| | gatctaccatacatcaagattacactcaggagtcccatcaaggttcagtg | P2A-SP- |
| | gcagtgggtctggaacagattattctctcaccattagcaacctggagcaa | IL15- |
| | gaagatattgccacttacttttgccaacagggtaatacgcttccgtacac | Anchoring |
| | gttcggaggggggaccaagctggagatcacaggtggcggtggctcgggcg | structure Full |
| | gtggtgggtcgggtggcggcggatctgaggtgaaactgcaggagtcagga | construct |
| | cctggcctggtggcgccctcacagagcctgtccgtcacatgcactgtctc | |
| | aggggtctcattacccgactatggtgtaagctggattcgccagcctccac | |
| | gaaagggtctggagtggctgggagtaatatggggtagtgaaaccacatac | |
| | tataattcagctctcaaatccagactgaccatcatcaaggacaactccaa | |
| | gagccaagttttcttaaaaatgaacagtctgcaaactgatgacacagcca | |
| | tttactactgtgccaaacattattactacggtggtagctatgctatggac | |
| | tactggggccaaggaacctcagtcaccgtctcctcaaccacgacgccagc | |
| | gccgcgaccaccaacaccggcgcccaccatcgcgtcgcagcccctgtccc | |
| | tgcgcccagaggcgtgccggccagcggcggggggcgcagtgcacacgagg | |
| | gggctggacttcgcctgtgatatctacatctgggcgcccttggccgggac | |
| | ttgtggggtccttctcctgtcactggttatcaccctttactgcaaacggg | |
| | gcagaaagaaactcctgtatatattcaaacaaccatttatgagaccagta | |
| | caaactactcaagaggaagatggctgtagctgccgatttccagaagaaga | |
| | agaaggaggatgtgaactgagagtgaagttcagcaggagcgcagacgccc | |
| | ccgcgtaccagcagggccagaaccagctctataacgagctcaatctagga | |
| | cgaagagaggagtacgatgttttggacaagagacgtggccgggaccctga | |
| | gatggggggaaagccgagaaggaagaaccctcaggaaggcctgtacaatg | |
| | aactgcagaaagataagatggcggaggcctacagtgagattgggatgaaa | |
| | ggcgagcgccggaggggcaaggggcacgatggcctttaccagggtctcag | |
| | tacagccaccaaggacacctacgacgcccttcacatgcaggccctgcccc | |
| | ctcgcggctccggcgagggcaggggaagtctactaacatgcggggacgtg | |
| | gaggaaaatcccggccccggtctcacctcccaactgcttccccctctgtt | |
| | cttcctgctagcatgtgccggcaactttgtccacggacacaagtgcgata | |
| | tcaccttacaggagatcatcaaaactttgaacagcctcacagagcagaag | |
| | actctgtgcaccgagttgaccgtaacagacatctttgctgcctccaagaa | |
| | cacaactgagaaggaaaccttctgcagggctgcgactgtgctccggcagt | |
| | tctacagccaccatgagaaggacactcgctgcctgggtgcgactgcacag | |
| | cagttccacaggcacaagcagctgatccgattcctgaaacggctcgacag | |
| | gaacctctggggcctggcgggcttgaattcctgtcctgtgaaggaagcca | |
| | accagagtacgttggaaaacttcttggaaaggctaaagacgatcatgaga | |
| | gagaaatattcaaagtgttcgagctctggaggaggaggatctggcggagg | |
| | aggaagtggaggaggaggctctggaggaggcggatctggaggagggagtc | |
| | tgcagcttgaaaatggtgggacatccttatcagagaaaacagttcttctg | |
| | ctggtgactccatttctggcagcagcctggagccttcatcccggaagcgg | |
| | agccacgaacttctctctgttaaagcaagcaggagatgttgaagaaaacc | |
| | ccgggcctggcaccagcctgctgtgctggatggcactgtgcctgctggga | |
| | gcagaccacgccgatgccggcatccacgtgttcatcctgggctgcttcag | |
| | cgccggacttcctaaaacagaggccaactgggtgaacgtgattagcgacc | |
| | tgaagaagatcgaggacctgatccagagcatgcacatcgacgccaccctg | |
| | tacaccgagagcgatgtgcatcctagctgcaaggtgaccgccatgaagtg | |
| | cttcctgctggagctgcaggtgatcagcctggagagcggagatgccagca | |
| | ttcacgacacagtggaaaatctgatcatcctggccaacaacagcctgagc | |
| | agcaacggcaatgtgaccgagagcggctgtaaggagtgcgaggaactgga | |
| | ggagaagaacatcaaggagttcctgcagagcttcgtgcacatcgtgcaga | |
| | tgttcatcaacaccagcagagcctctggaggaggaggatctggcggagga | |
| | ggaagtggaggaggaggctctggaggaggcggatctggaggagggagtct | |
| | gcagcttgaaaatggtgggacatccttatcagagaaaacagttcttctgc | |
| | tggtgactccatttctggcagcagcctggagccttcatccc | |
| 450 | gccttaccagtgaccgccttgctcctgccgctggccttgctgctccacgc | Leader |
| | cgccaggccg | (CD8α) |
| 451 | gacatccagatgacacagactacatcctccctgtctgcctctctgggaga | scFv (CD19) |
| | cagagtcaccatcagttgcagggcaagtcaggacattagtaaatatttaa | |
| | attggtatcagcagaaaccagatggaactgttaaactcctgatctaccat | |
| | acatcaagattacactcaggagtcccatcaaggttcagtggcagtgggtc | |
| | tggaacagattattctctcaccattagcaacctggagcaagaagatattg | |
| | ccacttacttttgccaacagggtaatacgcttccgtacacgttcggaggg | |
| | gggaccaagctggagatcacaggtggcggtggctcgggcggtggtgggtc | |
| | gggggcggcggatctgaggtgaaactgcaggagtcaggacctggcctgg | |
| | tggcgccctcacagagcctgtccgtcacatgcactgtctcaggggtctca | |
| | ttacccgactatggtgtaagctggattcgccagcctccacgaaagggtct | |
| | ggagtggctgggagtaatatggggtagtgaaaccacatactataattcag | |
| | ctctcaaatccagactgaccatcatcaaggacaactccaagagccaagtt | |
| | ttcttaaaaatgaacagtctgcaaactgatgacacagccatttactactg | |
| | tgccaaacattattactacggtggtagctatgctatggactactggggcc | |
| | aaggaacctcagtcaccgtctcctca | |
| 452 | accacgacgccagcgccgcgaccaccaacaccggcgcccaccatcgcgtc | Hinge |
| | gcagcccctgtccctgcgcccagaggcgtgccggccagcggcggggggcg | (CD8α) |
| | cagtgcacacgagggggctggacttcgcctgtgat | |
| 453 | atctacatctgggcgcccttggccgggacttgtggggtccttctcctgtc | Transmembrane |
| | actggttatcaccctttactgc | domain |
| | | (CD8a) |
| 454 | aaacggggcagaaagaaactcctgtatatattcaaacaaccatttatgag | Costimulatory |
| | accagtacaaactactcaagaggaagatggctgtagctgccgatttccag | domain (4- |
| | aagaagaagaaggaggatgtgaactg | 1BB) |
| 455 | agagtgaagttcagcaggagcgcagacgcccccgcgtaccagcagggcca | Intracellular |
| | gaaccagctctataacgagctcaatctaggacgaagagaggagtacgatg | signal domain |
| | ttttggacaagagacgtggccgggaccctgagatggggggaaagccgaga | (CD3ζ) |
| | aggaagaaccctcaggaaggcctgtacaatgaactgcagaaagataagat | |
| | ggcggaggcctacagtgagattgggatgaaaggcgagcgccggaggggca | |
| | aggggcacgatggcctttaccagggtctcagtacagccaccaaggacacc | |
| | tacgacgcccttcacatgcaggccctgccccctcgc | |
| 456 | gccttaccagtgaccgccttgctcctgccgctggccttgctgctccacgc | Complete |
| | cgccaggccggacatccagatgacacagactacatcctccctgtctgcct | sequence of |
| | ctctgggagacagagtcaccatcagttgcagggcaagtcaggacattagt | CAR (CD8α |
| | aaatatttaaattggtatcagcagaaaccagatggaactgttaaactcct | Leader + Anti |
| | gatctaccatacatcaagattacactcaggagtcccatcaaggttcagtg | CD19- |
| | gcagtgggtctggaacagattattctctcaccattagcaacctggagcaa | scFv + CD8α |
| | gaagatattgccacttacttttgccaacagggtaatacgcttccgtacac | Hinge + CD8α |
| | gttcggaggggggaccaagctggagatcacaggtggcggtggctcgggcg | Transmembrane |
| | gtggtgggtcgggtggcggcggatctgaggtgaaactgcaggagtcagga | + 4- |
| | cctggcctggtggcgccctcacagagcctgtccgtcacatgcactgtctc | 1BB + CD3ζ) |
| | aggggtctcattacccgactatggtgtaagctggattcgccagcctccac | |
| | gaaagggtctggagtggctgggagtaatatggggtagtgaaaccacatac | |
| | tataattcagctctcaaatccagactgaccatcatcaaggacaactccaa | |
| | gagccaagttttcttaaaaatgaacagtctgcaaactgatgacacagcca | |
| | tttactactgtgccaaacattattactacggtggtagctatgctatggac | |
| | tactggggccaaggaacctcagtcaccgtctcctcaaccacgacgccagc | |
| | gccgcgaccaccaacaccggcgcccaccatcgcgtcgcagcccctgtccc | |
| | tgcgcccagaggcgtgccggccagcggcggggggcgcagtgcacacgagg | |
| | gggctggacttcgcctgtgatatctacatctgggcgcccttggccgggac | |
| | ttgtggggtccttctcctgtcactggttatcaccctttactgcaaacggg | |
| | gcagaaagaaactcctgtatatattcaaacaaccatttatgagaccagta | |
| | caaactactcaagaggaagatggctgtagctgccgatttccagaagaaga | |
| | agaaggaggatgtgaactgagagtgaagttcagcaggagcgcagacgccc | |
| | ccgcgtaccagcagggccagaaccagctctataacgagctcaatctagga | |
| | cgaagagaggagtacgatgttttggacaagagacgtggccgggaccctga | |
| | gatggggggaaagccgagaaggaagaaccctcaggaaggcctgtacaatg | |
| | aactgcagaaagataagatggcggaggcctacagtgagattgggatgaaa | |
| | ggcgagcgccggaggggcaaggggcacgatggcctttaccagggtctcag | |
| | tacagccaccaaggacacctacgacgcccttcacatgcaggccctgcccc | |
| | ctcgc | |
| | ggctccggc | linker |
| 351 | gagggcaggggaagtctactaacatgcggggacgtggaggaaaatcccgg | T2A peptide |
| | CCCC | |
| 58 | ggtctcacctcccaactgcttccccctctgttcttcctgctagcatgtgc | Signal (IL-4) |
| | cggcaactttgtccacgga | |
| 163 | cacaagtgcgatatcaccttacaggagatcatcaaaactttgaacagcct | IL-4 |
| | cacagagcagaagactctgtgcaccgagttgaccgtaacagacatctttg | |
| | ctgcctccaagaacacaactgagaaggaaaccttctgcagggctgcgact | |
| | gtgctccggcagttctacagccaccatgagaaggacactcgctgcctggg | |
| | tgcgactgcacagcagttccacaggcacaagcagctgatccgattcctga | |
| | aacggctcgacaggaacctctggggcctggcgggcttgaattcctgtcct | |
| | gtgaaggaagccaaccagagtacgttggaaaacttcttggaaaggctaaa | |
| | gacgatcatgagagagaaatattcaaagtgttcgagc | |
| 509 | tctggaggaggaggatctggcggaggaggaagtggaggaggaggctctgg | Lr1 |
| | aggaggcggatctggaggagggagtctgcag | |
| 250 | cttgaaaatggtgggacatccttatcagagaaaacagttcttctgctggt | GPI- |
| | gactccatttctggcagcagcctggagccttcatccc | attachment |
| | | signal (CD59) |
| | ggaagcgga | linker |
| 355 | gccacgaacttctctctgttaaagcaagcaggagatgttgaagaaaaccc | P2A |
| | cgggcct | |
| 53 | ggcaccagcctgctgtgctggatggcactgtgcctgctgggagcagacca | Signal (TCR) |
| | cgccgatgcc | |
| 156 | ggcatccacgtgttcatcctgggctgcttcagcgccggacttcctaaaac | IL-15 |
| | agaggccaactgggtgaacgtgattagcgacctgaagaagatcgaggacc | |
| | tgatccagagcatgcacatcgacgccaccctgtacaccgagagcgatgtg | |
| | catcctagctgcaaggtgaccgccatgaagtgcttcctgctggagctgca | |
| | ggtgatcagcctggagagcggagatgccagcattcacgacacagtggaaa | |
| | atctgatcatcctggccaacaacagcctgagcagcaacggcaatgtgacc | |
| | gagagcggctgtaaggagtgcgaggaactggaggagaagaacatcaagga | |
| | gttcctgcagagcttcgtgcacatcgtgcagatgttcatcaacaccagca | |
| | gagcc | |
| 509 | tctggaggaggaggatctggcggaggaggaagtggaggaggaggctctgg | Lr1 |
| | aggaggcggatctggaggagggagtctgcag | |
| 250 | cttgaaaatggtgggacatccttatcagagaaaacagttcttctgctggt | GPI- |
| | gactccatttctggcagcagcctggagccttcatccc | attachment |
| | | signal (CD59) |

Exemplary molecule 9 CAR19-T2A-SP-IL4-Lr1-Ar1-P2A-SP-IL15- Lr1-Arl

| | | |
|---|---|---|
| 528 | gccttaccagtgaccgccttgctcctgccgctggccttgctgctccacgc | CAR19-T2A- |
| | cgccaggccggacatccagatgacacagactacatcctccctgtctgcct | SP-IL4-Lr1- |
| | ctctgggagacagagtcaccatcagttgcagggcaagtcaggacattagt | Ar1-P2A-SP- |
| | aaatatttaaattggtatcagcagaaaccagatggaactgttaaactcct | IL15-Lr1- |
| | gatctaccatacatcaagattacactcaggagtcccatcaaggttcagtg | Arl full |
| | gcagtgggtctggaacagattattctctcaccattagcaacctggagcaa | construct |
| | gaagatattgccacttacttttgccaacagggtaatacgcttccgtacac | |
| | gttcggaggggggaccaagctggagatcacaggtggcggtggctcgggcg | |
| | gtggtgggtcgggtggcggcggatctgaggtgaaactgcaggagtcagga | |
| | cctggcctggtggcgccctcacagagcctgtccgtcacatgcactgtctc | |
| | aggggtctcattacccgactatggtgtaagctggattcgccagcctccac | |
| | gaaagggtctggagtggctgggagtaatatggggtagtgaaaccacatac | |
| | tataattcagctctcaaatccagactgaccatcatcaaggacaactccaa | |
| | gagccaagttttcttaaaaatgaacagtctgcaaactgatgacacagcca | |
| | tttactactgtgccaaacattattactacggtggtagctatgctatggac | |
| | tactggggccaaggaacctcagtcaccgtctcctcaaccacgacgccagc | |
| | gccgcgaccaccaacaccggcgcccaccatcgcgtcgcagcccctgtccc | |
| | tgcgcccagaggcgtgccggccagcggcggggggcgcagtgcacacgagg | |
| | gggctggacttcgcctgtgatatctacatctgggcgcccttggccgggac | |
| | ttgtggggtccttctcctgtcactggttatcaccctttactgcaaacggg | |
| | gcagaaagaaactcctgtatatattcaaacaaccatttatgagaccagta | |
| | caaactactcaagaggaagatggctgtagctgccgatttccagaagaaga | |
| | agaaggaggatgtgaactgagagtgaagttcagcaggagcgcagacgccc | |
| | ccgcgtaccagcagggccagaaccagctctataacgagctcaatctagga | |
| | cgaagagaggagtacgatgttttggacaagagacgtggccgggaccctga | |
| | gatggggggaaagccgagaaggaagaaccctcaggaaggcctgtacaatg | |
| | aactgcagaaagataagatggcggaggcctacagtgagattgggatgaaa | |
| | ggcgagcgccggaggggcaaggggcacgatggcctttaccagggtctcag | |
| | tacagccaccaaggacacctacgacgcccttcacatgcaggccctgcccc | |
| | ctcgcggctccggcgagggcaggggaagtctactaacatgcggggacgtg | |
| | gaggaaaatcccggccccggtctcacctcccaactgcttccccctctgtt | |
| | cttcctgctagcatgtgccggcaactttgtccacggacacaagtgcgata | |
| | tcaccttacaggagatcatcaaaactttgaacagcctcacagagcagaag | |
| | actctgtgcaccgagttgaccgtaacagacatctttgctgcctccaagaa | |
| | cacaactgagaaggaaaccttctgcagggctgcgactgtgctccggcagt | |
| | tctacagccaccatgagaaggacactcgctgcctgggtgcgactgcacag | |
| | cagttccacaggcacaagcagctgatccgattcctgaaacggctcgacag | |
| | gaacctctggggcctggcgggcttgaattcctgtcctgtgaaggaagcca | |
| | accagagtacgttggaaaacttcttggaaaggctaaagacgatcatgaga | |
| | gagaaatattcaaagtgttcgagctctggaggaggaggatctggcggagg | |
| | aggaagtggaggaggaggctctggaggaggcggatctggaggagggagtc | |
| | tgcagatctacatctgggctcctctggctggcacctgcggagtgctgctg | |
| | ctgtctctggtgattactggaagcggagccacgaacttctctctgttaaa | |
| | gcaagcaggagatgttgaagaaaaccccgggcctggcaccagcctgctgt | |
| | gctggatggcactgtgcctgctgggagcagaccacgccgatgccggcatc | |
| | cacgtgttcatcctgggctgcttcagcgccggacttcctaaaacagaggc | |
| | caactgggtgaacgtgattagcgacctgaagaagatcgaggacctgatcc | |
| | agagcatgcacatcgacgccaccctgtacaccgagagcgatgtgcatcct | |
| | agctgcaaggtgaccgccatgaagtgcttcctgctggagctgcaggtgat | |
| | cagcctggagagcggagatgccagcattcacgacacagtggaaaatctga | |
| | tcatcctggccaacaacagcctgagcagcaacggcaatgtgaccgagagc | |
| | ggctgtaaggagtgcgaggaactggaggagaagaacatcaaggagttcct | |
| | gcagagcttcgtgcacatcgtgcagatgttcatcaacaccagcagagcct | |
| | ctggaggaggaggatctggcggaggaggaagtggaggaggaggctctgga | |
| | ggaggcggatctggaggagggagtctgcagatctacatctgggctcctct | |
| | ggctggcacctgcggagtgctgctgctgtctctggtgattact | |
| 450 | gccttaccagtgaccgccttgctcctgccgctggccttgctgctccacgc | Leader |
| | cgccaggccg | (CD8α) |
| 451 | gacatccagatgacacagactacatcctccctgtctgcctctctgggaga | scFv (CD19) |
| | cagagtcaccatcagttgcagggcaagtcaggacattagtaaatatttaa | |
| | attggtatcagcagaaaccagatggaactgttaaactcctgatctaccat | |
| | acatcaagattacactcaggagtcccatcaaggttcagtggcagtgggtc | |
| | tggaacagattattctctcaccattagcaacctggagcaagaagatattg | |
| | ccacttacttttgccaacagggtaatacgcttccgtacacgttcggaggg | |
| | gggaccaagctggagatcacaggtggcggtggctcgggcggtggtgggtc | |
| | gggtggcggcggatctgaggtgaaactgcaggagtcaggacctggcctgg | |
| | tggcgccctcacagagcctgtccgtcacatgcactgtctcaggggtctca | |
| | ttacccgactatggtgtaagctggattcgccagcctccacgaaagggtct | |
| | ggagtggctgggagtaatatggggtagtgaaaccacatactataattcag | |
| | ctctcaaatccagactgaccatcatcaaggacaactccaagagccaagtt | |
| | ttcttaaaaatgaacagtctgcaaactgatgacacagccatttactactg | |
| | tgccaaacattattactacggtggtagctatgctatggactactggggcc | |
| | aaggaacctcagtcaccgtctcctca | |
| 452 | accacgacgccagcgccgcgaccaccaacaccggcgcccaccatcgcgtc | Hinge |
| | gcagcccctgtccctgcgcccagaggcgtgccggccagcggcggggggcg | (CD8α) |
| | cagtgcacacgagggggctggacttcgcctgtgat | |
| 453 | atctacatctgggcgcccttggccgggacttgtggggtccttctcctgtc | Transmembrane |
| | actggttatcaccctttactgc | domain |
| | | (CD8α) |
| 454 | aaacggggcagaaagaaactcctgtatatattcaaacaaccatttatgag | Costimulatory |
| | accagtacaaactactcaagaggaagatggctgtagctgccgatttccag | domain (4- |
| | aagaagaagaaggaggatgtgaactg | 1BB) |
| 455 | agagtgaagttcagcaggagcgcagacgcccccgcgtaccagcagggcca | Intracellular |
| | gaaccagctctataacgagctcaatctaggacgaagagaggagtacgatg | signal domain |
| | ttttggacaagagacgtggccgggaccctgagatggggggaaagccgaga | (CD3ζ) |
| | aggaagaaccctcaggaaggcctgtacaatgaactgcagaaagataagat | |
| | ggcggaggcctacagtgagattgggatgaaaggcgagcgccggaggggca | |
| | aggggcacgatggcctttaccagggtctcagtacagccaccaaggacacc | |
| | tacgacgcccttcacatgcaggccctgccccctcgc | |
| 456 | gccttaccagtgaccgccttgctcctgccgctggccttgctgctccacgc | Complete |
| | cgccaggccggacatccagatgacacagactacatcctccctgtctgcct | sequence of |
| | ctctgggagacagagtcaccatcagttgcagggcaagtcaggacattagt | CAR (CD8α |
| | aaatatttaaattggtatcagcagaaaccagatggaactgttaaactcct | Leader + Anti |
| | gatctaccatacatcaagattacactcaggagtcccatcaaggttcagtg | CD19- |
| | gcagtgggtctggaacagattattctctcaccattagcaacctggagcaa | scFv + CD8α |
| | gaagatattgccacttacttttgccaacagggtaatacgcttccgtacac | Hinge + CD8α |
| | gttcggaggggggaccaagctggagatcacaggtggcggtggctcgggcg | Transmembrane |
| | gtggtgggtcgggtggcggcggatctgaggtgaaactgcaggagtcagga | + 4- |
| | cctggcctggtggcgccctcacagagcctgtccgtcacatgcactgtctc | 1BB+CD3ζ) |
| | aggggtctcattacccgactatggtgtaagctggattcgccagcctccac | |
| | gaaagggtctggagtggctgggagtaatatggggtagtgaaaccacatac | |
| | tataattcagctctcaaatccagactgaccatcatcaaggacaactccaa | |
| | gagccaagttttcttaaaaatgaacagtctgcaaactgatgacacagcca | |
| | tttactactgtgccaaacattattactacggtggtagctatgctatggac | |
| | tactggggccaaggaacctcagtcaccgtctcctcaaccacgacgccagc | |
| | gccgcgaccaccaacaccggcgcccaccatcgcgtcgcagcccctgtccc | |
| | tgcgcccagaggcgtgccggccagcggcggggggcgcagtgcacacgagg | |
| | gggctggacttcgcctgtgatatctacatctgggcgcccttggccgggac | |
| | ttgtggggtccttctcctgtcactggttatcaccctttactgcaaacggg | |
| | gcagaaagaaactcctgtatatattcaaacaaccatttatgagaccagta | |
| | caaactactcaagaggaagatggctgtagctgccgatttccagaagaaga | |
| | agaaggaggatgtgaactgagagtgaagttcagcaggagcgcagacgccc | |
| | ccgcgtaccagcagggccagaaccagctctataacgagctcaatctagga | |
| | cgaagagaggagtacgatgttttggacaagagacgtggccgggaccctga | |
| | gatggggggaaagccgagaaggaagaaccctcaggaaggcctgtacaatg | |
| | aactgcagaaagataagatggcggaggcctacagtgagattgggatgaaa | |
| | ggcgagcgccggaggggcaaggggcacgatggcctttaccagggtctcag | |
| | tacagccaccaaggacacctacgacgcccttcacatgcaggccctgcccc | |
| | ctcgc | |
| | ggctccggc | linker |
| 351 | gagggcaggggaagtctactaacatgcggggacgtggaggaaaatcccgg | T2A peptide |
| | cccc | |
| 58 | ggtctcacctcccaactgcttccccctctgttcttcctgctagcatgtgc | Signal (IL-4) |
| | cggcaactttgtccacgga | |
| 163 | cacaagtgcgatatcaccttacaggagatcatcaaaactttgaacagcct | IL-4 |
| | cacagagcagaagactctgtgcaccgagttgaccgtaacagacatctttg | |
| | ctgcctccaagaacacaactgagaaggaaaccttctgcagggctgcgact | |
| | gtgctccggcagttctacagccaccatgagaaggacactcgctgcctggg | |
| | tgcgactgcacagcagttccacaggcacaagcagctgatccgattcctga | |
| | aacggctcgacaggaacctctggggcctggcgggcttgaattcctgtcct | |
| | gtgaaggaagccaaccagagtacgttggaaaacttcttggaaaggctaaa | |
| | gacgatcatgagagagaaatattcaaagtgttcgagc | |
| 509 | tctggaggaggaggatctggcggaggaggaagtggaggaggaggctctgg | Lr1 |
| | aggaggcggatctggaggagggagtctgcag | |
| 253 | atctacatctgggctcctctggctggcacctgcggagtgctgctgctgtc | transmembrane |
| | tctggtgattact | peptide |
| | | (Ar1) |
| | ggaagcgga | linker |
| 355 | gccacgaacttctctctgttaaagcaagcaggagatgttgaagaaaaccc | P2A |
| | cgggcct | |
| 53 | ggcaccagcctgctgtgctggatggcactgtgcctgctgggagcagacca | Signal (TCR) |
| | cgccgatgcc | |
| 156 | ggcatccacgtgttcatcctgggctgcttcagcgccggacttcctaaaac | IL-15 |
| | agaggccaactgggtgaacgtgattagcgacctgaagaagatcgaggacc | |
| | tgatccagagcatgcacatcgacgccaccctgtacaccgagagcgatgtg | |
| | catcctagctgcaaggtgaccgccatgaagtgcttcctgctggagctgca | |
| | ggtgatcagcctggagagcggagatgccagcattcacgacacagtggaaa | |
| | atctgatcatcctggccaacaacagcctgagcagcaacggcaatgtgacc | |
| | gagagcggctgtaaggagtgcgaggaactggaggagaagaacatcaagga | |
| | gttcctgcagagcttcgtgcacatcgtgcagatgttcatcaacaccagca | |
| | gagcc | |
| 509 | tctggaggaggaggatctggcggaggaggaagtggaggaggaggctctgg | Lr1 |
| | aggaggcggatctggaggagggagtctgcag | |
| 253 | atctacatctgggctcctctggctggcacctgcggagtgctgctgctgtc | transmembrane |
| | tctggtgattact | peptide |
| | | (Ar1) |

Exemplary molecule 10 SP-IL4-Anchoring structure-T2A-SP-IL15-Anchoring structure

| | | |
|---|---|---|
| 529 | ggtctcacctcccaactgcttccccctctgttcttcctgctagcatgtgc | SP-IL4- |
| | cggcaactttgtccacggacacaagtgcgatatcaccttacaggagatca | Anchoring |
| | tcaaaactttgaacagcctcacagagcagaagactctgtgcaccgagttg | structure- |
| | accgtaacagacatctttgctgcctccaagaacacaactgagaaggaaac | T2A-SP- |
| | cttctgcagggctgcgactgtgctccggcagttctacagccaccatgaga | IL15- |
| | aggacactcgctgcctgggtgcgactgcacagcagttccacaggcacaag | Anchoring |
| | cagctgatccgattcctgaaacggctcgacaggaacctctggggcctggc | structure full |
| | gggcttgaattcctgtcctgtgaaggaagccaaccagagtacgttggaaa | construct |
| | acttcttggaaaggctaaagacgatcatgagagagaaatattcaaagtgt | |
| | tcgagctctggaggaggaggatctggcggaggaggaagtggaggaggagg | |
| | ctctggaggaggcggatctggaggagggagtctgcagcttgaaaatggtg | |
| | ggacatccttatcagagaaaacagttcttctgctggtgactccatttctg | |
| | gcagcagcctggagccttcatcccggctccggcgagggcaggggaagtct | |
| | actaacatgcggggacgtggaggaaaatcccggccccggcaccagcctgc | |
| | tgtgctggatggcactgtgcctgctgggagcagaccacgccgatgccggc | |
| | atccacgtgttcatcctgggctgcttcagcgccggacttcctaaaacaga | |
| | ggccaactgggtgaacgtgattagcgacctgaagaagatcgaggacctga | |
| | tccagagcatgcacatcgacgccaccctgtacaccgagagcgatgtgcat | |
| | cctagctgcaaggtgaccgccatgaagtgcttcctgctggagctgcaggt | |
| | gatcagcctggagagcggagatgccagcattcacgacacagtggaaaatc | |
| | tgatcatcctggccaacaacagcctgagcagcaacggcaatgtgaccgag | |
| | agcggctgtaaggagtgcgaggaactggaggagaagaacatcaaggagtt | |
| | cctgcagagcttcgtgcacatcgtgcagatgttcatcaacaccagcagag | |
| | cctctggaggaggaggatctggcggaggaggaagtggaggaggaggctct | |
| | ggaggaggcggatctggaggagggagtctgcagcttgaaaatggtgggac | |
| | atccttatcagagaaaacagttcttctgctggtgactccatttctggcag | |
| | cagcctggagccttcatccc | |
| 58 | ggtctcacctcccaactgcttccccctctgttcttcctgctagcatgtgc | Signal (IL-4) |
| | cggcaactttgtccacgga | |
| 163 | cacaagtgcgatatcaccttacaggagatcatcaaaactttgaacagcct | IL-4 |
| | cacagagcagaagactctgtgcaccgagttgaccgtaacagacatctttg | |
| | ctgcctccaagaacacaactgagaaggaaaccttctgcagggctgcgact | |
| | gtgctccggcagttctacagccaccatgagaaggacactcgctgcctggg | |
| | tgcgactgcacagcagttccacaggcacaagcagctgatccgattcctga | |
| | aacggctcgacaggaacctctggggcctggcgggcttgaattcctgtcct | |
| | gtgaaggaagccaaccagagtacgttggaaaacttcttggaaaggctaaa | |
| | gacgatcatgagagagaaatattcaaagtgttcgagc | |
| 509 | tctggaggaggaggatctggcggaggaggaagtggaggaggaggctctgg | Lr1 |
| | aggaggcggatctggaggagggagtctgcag | |
| 250 | cttgaaaatggtgggacatccttatcagagaaaacagttcttctgctggt | GPI- |
| | gactccatttctggcagcagcctggagccttcatccc | attachment |
| | | signal (CD59) |
| | ggctccggc | linker |
| 351 | gagggcaggggaagtctactaacatgcggggacgtggaggaaaatcccgg | T2A peptide |
| | CCCC | |
| 53 | ggcaccagcctgctgtgctggatggcactgtgcctgctgggagcagacca | Signal (TCR) |
| | cgccgatgcc | |
| 156 | ggcatccacgtgttcatcctgggctgcttcagcgccggacttcctaaaac | IL-15 |
| | agaggccaactgggtgaacgtgattagcgacctgaagaagatcgaggacc | |
| | tgatccagagcatgcacatcgacgccaccctgtacaccgagagcgatgtg | |
| | catcctagctgcaaggtgaccgccatgaagtgcttcctgctggagctgca | |
| | ggtgatcagcctggagagcggagatgccagcattcacgacacagtggaaa | |
| | atctgatcatcctggccaacaacagcctgagcagcaacggcaatgtgacc | |
| | gagagcggctgtaaggagtgcgaggaactggaggagaagaacatcaagga | |
| | gttcctgcagagcttcgtgcacatcgtgcagatgttcatcaacaccagca | |
| | gagcc | |
| 509 | tctggaggaggaggatctggcggaggaggaagtggaggaggaggctctgg | Lr1 |
| | aggaggcggatctggaggagggagtctgcag | |
| 250 | cttgaaaatggtgggacatccttatcagagaaaacagttcttctgctggt | GPI- |
| | gactccatttctggcagcagcctggagccttcatccc | attachment |
| | | signal (CD59) |

Exemplary molecule 11 SP-IL4-Lr1-Ar1-T2A-SP-IL15-Lr1-Arl

| | | |
|---|---|---|
| 533 | ggtctcacctcccaactgcttccccctctgttcttcctgctagcatgtgc | SP-IL4-Lr1- |
| | cggcaactttgtccacggacacaagtgcgatatcaccttacaggagatca | Ar1-T2A-SP- |
| | tcaaaactttgaacagcctcacagagcagaagactctgtgcaccgagttg | IL15-Lr1-Ar1 |
| | accgtaacagacatctttgctgcctccaagaacacaactgagaaggaaac | full construct |
| | cttctgcagggctgcgactgtgctccggcagttctacagccaccatgaga | |
| | aggacactcgctgcctgggtgcgactgcacagcagttccacaggcacaag | |
| | cagctgatccgattcctgaaacggctcgacaggaacctctggggcctggc | |
| | gggcttgaattcctgtcctgtgaaggaagccaaccagagtacgttggaaa | |
| | acttcttggaaaggctaaagacgatcatgagagagaaatattcaaagtgt | |
| | tcgagctctggaggaggaggatctggcggaggaggaagtggaggaggagg | |
| | ctctggaggaggcggatctggaggagggagtctgcagatctacatctggg | |
| | ctcctctggctggcacctgcggagtgctgctgctgtctctggtgattact | |
| | ggctccggcgagggcaggggaagtctactaacatgcggggacgtggagga | |
| | aaatcccggccccggcaccagcctgctgtgctggatggcactgtgcctgc | |
| | tgggagcagaccacgccgatgccggcatccacgtgttcatcctgggctgc | |
| | ttcagcgccggacttcctaaaacagaggccaactgggtgaacgtgattag | |
| | cgacctgaagaagatcgaggacctgatccagagcatgcacatcgacgcca | |
| | ccctgtacaccgagagcgatgtgcatcctagctgcaaggtgaccgccatg | |
| | aagtgcttcctgctggagctgcaggtgatcagcctggagagcggagatgc | |
| | cagcattcacgacacagtggaaaatctgatcatcctggccaacaacagcc | |
| | tgagcagcaacggcaatgtgaccgagagcggctgtaaggagtgcgaggaa | |
| | ctggaggagaagaacatcaaggagttcctgcagagcttcgtgcacatcgt | |
| | gcagatgttcatcaacaccagcagagcctctggaggaggaggatctggcg | |
| | gaggaggaagtggaggaggaggctctggaggaggcggatctggaggaggg | |
| | agtctgcagatctacatctgggctcctctggctggcacctgcggagtgct | |
| | gctgctgtctctggtgattact | |
| 58 | ggtctcacctcccaactgcttccccctctgttcttcctgctagcatgtgc | Signal (IL-4) |
| | cggcaactttgtccacgga | |
| 163 | cacaagtgcgatatcaccttacaggagatcatcaaaactttgaacagcct | IL-4 |
| | cacagagcagaagactctgtgcaccgagttgaccgtaacagacatctttg | |
| | ctgcctccaagaacacaactgagaaggaaaccttctgcagggctgcgact | |
| | gtgctccggcagttctacagccaccatgagaaggacactcgctgcctggg | |
| | tgcgactgcacagcagttccacaggcacaagcagctgatccgattcctga | |
| | aacggctcgacaggaacctctggggcctggcgggcttgaattcctgtcct | |
| | gtgaaggaagccaaccagagtacgttggaaaacttcttggaaaggctaaa | |
| | gacgatcatgagagagaaatattcaaagtgttcgagc | |
| 509 | tctggaggaggaggatctggcggaggaggaagtggaggaggaggctctgg | Lr1 |
| | aggaggcggatctggaggagggagtctgcag | |
| 253 | atctacatctgggctcctctggctggcacctgcggagtgctgctgctgtc | transmembrane |
| | tctggtgattact | peptide |
| | | (Ar1) |
| | ggctccggc | linker |
| 53 | ggcaccagcctgctgtgctggatggcactgtgcctgctgggagcagacca | Signal (TCR) |
| | cgccgatgcc | |
| 156 | ggcatccacgtgttcatcctgggctgcttcagcgccggacttcctaaaac | IL-15 |
| | agaggccaactgggtgaacgtgattagcgacctgaagaagatcgaggacc | |
| | tgatccagagcatgcacatcgacgccaccctgtacaccgagagcgatgtg | |
| | catcctagctgcaaggtgaccgccatgaagtgcttcctgctggagctgca | |
| | ggtgatcagcctggagagcggagatgccagcattcacgacacagtggaaa | |
| | atctgatcatcctggccaacaacagcctgagcagcaacggcaatgtgacc | |
| | gagagcggctgtaaggagtgcgaggaactggaggagaagaacatcaagga | |
| | gttcctgcagagcttcgtgcacatcgtgcagatgttcatcaacaccagca | |
| | gagcc | |
| 509 | tctggaggaggaggatctggcggaggaggaagtggaggaggaggctctgg | Lr1 |
| | aggaggcggatctggaggagggagtctgcag | |
| 253 | atctacatctgggctcctctggctggcacctgcggagtgctgctgctgtc | transmembrane |
| | tctggtgattact | peptide |
| | | (Ar1) |

TABLE A-11
AMINO ACID SEQUENCES OF VIRAL VECTOR CONSTRUCTS WITH CAR
AND TWO CYTOKINES
Exemplary molecule 8 CAR19-T2A-SP-IL4-Anchoring structure-P2A-SP-IL15-Anchoring structure

| SEQ | | |
|---|---|---|
| ID NO: | Sequence | Description |
| 530 | ALPVTALLLPLALLLHAARPDIQMTQTTSSLSASLGDRVTISCRASQDI | CAR19-T2A- |
| | SKYLNWYQQKPDGTVKLLIYHTSRLHSGVPSRFSGSGSGTDYSLTISNL | SP-IL4- |
| | EQEDIATYFCQQGNTLPYTFGGGTKLEITGGGGSGGGGSGGGGSEVKLQ | Anchoring |
| | ESGPGLVAPSQSLSVTCTVSGVSLPDYGVSWIRQPPRKGLEWLGVIWGS | structure-P2A- |
| | ETTYYNSALKSRLTIIKDNSKSQVFLKMNSLQTDDTAIYYCAKHYYYGG | SP-IL15- |
| | SYAMDYWGQGTSVTVSSTTTPAPRPPTPAPTIASQPLSLRPEACRPAAG | Anchoring |
| | GAVHTRGLDFACDIYIWAPLAGTCGVLLLSLVITLYCKRGRKKLLYIFK | structure Full |
| | QPFMRPVQTTQEEDGCSCRFPEEEEGGCELRVKFSRSADAPAYQQGQNQ | construct |
| | LYNELNLGRREEYDVLDKRRGRDPEMGGKPRRKNPQEGLYNELQKDKMA | |
| | EAYSEIGMKGERRRGKGHDGLYQGLSTATKDTYDALHMQALPPRGSGEG | |
| | RGSLLTCGDVEENPGPGLTSQLLPPLFFLLACAGNFVHGHKCDITLQEI | |
| | IKTLNSLTEQKTLCTELTVTDIFAASKNTTEKETFCRAATVLRQFYSHH | |
| | EKDTRCLGATAQQFHRHKQLIRFLKRLDRNLWGLAGLNSCPVKEANQST | |
| | LENFLERLKTIMREKYSKCSSSGGGGSGGGGSGGGGSGGGGSGGGSLQL | |
| | ENGGTSLSEKTVLLLVTPFLAAAWSLHPGSGATNFSLLKQAGDVEENPG | |
| | PGTSLLCWMALCLLGADHADAGIHVFILGCFSAGLPKTEANWVNVISDL | |
| | KKIEDLIQSMHIDATLYTESDVHPSCKVTAMKCFLLELQVISLESGDAS | |
| | IHDTVENLIILANNSLSSNGNVTESGCKECEELEEKNIKEFLQSFVHIV | |
| | QMFINTSRASGGGGSGGGGSGGGGSGGGGSGGGSLQLENGGTSLSEKTV | |
| | LLLVTPFLAAAWSLHP | |
| 400 | ALPVTALLLPLALLLHAARP | Leader (CD8α) |
| 401 | DIQMTQTTSSLSASLGDRVTISCRASQDISKYLNWYQQKPDGTVKLLIY | scFv (CD19) |
| | HTSRLHSGVPSRFSGSGSGTDYSLTISNLEQEDIATYFCQQGNTLPYTF | |
| | GGGTKLEITGGGGSGGGGSGGGGSEVKLQESGPGLVAPSQSLSVTCTVS | |
| | GVSLPDYGVSWIRQPPRKGLEWLGVIWGSETTYYNSALKSRLTIIKDNS | |
| | KSQVFLKMNSLQTDDTAIYYCAKHYYYGGSYAMDYWGQGTSVTVSS | |
| 402 | TTTPAPRPPTPAPTIASQPLSLRPEACRPAAGGAVHTRGLDFACD | Hinge (CD8α) |
| 403 | IYIWAPLAGTCGVLLLSLVITLYC | Transmembrane |
| | | domain (CD8α) |
| 404 | KRGRKKLLYIFKQPFMRPVQTTQEEDGCSCRFPEEEEGGCEL | Costimulatory |
| | | domain (4- |
| | | 1BB) |
| 405 | RVKFSRSADAPAYQQGQNQLYNELNLGRREEYDVLDKRRGRDPEMGGKP | Intracellular |
| | RRKNPQEGLYNELQKDKMAEAYSEIGMKGERRRGKGHDGLYQGLSTATK | signal domain |
| | DTYDALHMQALPPR | (CD3ζ) |
| 406 | ALPVTALLLPLALLLHAARPDIQMTQTTSSLSASLGDRVTISCRASQDI | Complete |
| | SKYLNWYQQKPDGTVKLLIYHTSRLHSGVPSRFSGSGSGTDYSLTISNL | sequence of |
| | EQEDIATYFCQQGNTLPYTFGGGTKLEITGGGGSGGGGSGGGGSEVKLQ | CAR (CD8α |
| | ESGPGLVAPSQSLSVTCTVSGVSLPDYGVSWIRQPPRKGLEWLGVIWGS | Leader + Anti |
| | ETTYYNSALKSRLTIIKDNSKSQVFLKMNSLQTDDTAIYYCAKHYYYGG | CD19-scFv + |
| | SYAMDYWGQGTSVTVSSTTTPAPRPPTPAPTIASQPLSLRPEACRPAAG | CD8α Hinge + |
| | GAVHTRGLDFACDIYIWAPLAGTCGVLLLSLVITLYCKRGRKKLLYIFK | CD8α |
| | QPFMRPVQTTQEEDGCSCRFPEEEEGGCELRVKFSRSADAPAYQQGQNQ | Transmembrane |
| | LYNELNLGRREEYDVLDKRRGRDPEMGGKPRRKNPQEGLYNELQKDKMA | + 4-1BB + |
| | EAYSEIGMKGERRRGKGHDGLYQGLSTATKDTYDALHMQALPPR | CD3ζ) |
| | GSG | linker |
| 301 | EGRGSLLTCGDVEENPGP | T2A peptide |
| 113 | GLTSQLLPPLFFLLACAGNFVHG | Signal (IL-4) |
| 101 | HKCDITLQEIIKTLNSLTEQKTLCTELTVTDIFAASKNTTEKETFCRAA | IL-4 |
| | TVLRQFYSHHEKDTRCLGATAQQFHRHKQLIRFLKRLDRNLWGLAGLNS | |
| | CPVKEANQSTLENFLERLKTIMREKYSKCSS | |
| 506 | SGGGGSGGGGSGGGGSGGGGSGGGSLQ | Lr1 |
| 200 | LENGGTSLSEKTVLLLVTPFLAAAWSLHP | GPI-attachment |
| | | signal (CD59) |
| | GSG | Linker |
| 300 | ATNFSLLKQAGDVEENPGP | P2A peptide |
| 3 | GTSLLCWMALCLLGADHADA | Signal (TCR) |
| 106 | GIHVFILGCFSAGLPKTEANWVNVISDLKKIEDLIQSMHIDATLYTESD | IL-15 |
| | VHPSCKVTAMKCFLLELQVISLESGDASIHDTVENLIILANNSLSSNGN | |
| | VTESGCKECEELEEKNIKEFLQSFVHIVQMFINTSRA | |
| 506 | SGGGGSGGGGSGGGGSGGGGSGGGSLQ | Lr1 |
| 200 | LENGGTSLSEKTVLLLVTPFLAAAWSLHP | GPI-attachment |
| | | signal (CD59) |

Exemplary molecule 10 CAR19-T2A-SP-IL4-Lr1-Ar1-P2A-SP-IL15- Lr1-Ar1

| | | |
|---|---|---|
| 531 | ALPVTALLLPLALLLHAARPDIQMTQTTSSLSASLGDRVTISCRASQDI | CAR19-T2A- |
| | SKYLNWYQQKPDGTVKLLIYHTSRLHSGVPSRFSGSGSGTDYSLTISNL | SP-IL4-Lr1- |
| | EQEDIATYFCQQGNTLPYTFGGGTKLEITGGGGSGGGGSGGGGSEVKLQ | Ar1-P2A-SP- |
| | ESGPGLVAPSQSLSVTCTVSGVSLPDYGVSWIRQPPRKGLEWLGVIWGS | IL15-Lr1-Arl |
| | ETTYYNSALKSRLTIIKDNSKSQVFLKMNSLQTDDTAIYYCAKHYYYGG | full construct |
| | SYAMDYWGQGTSVTVSSTTTPAPRPPTPAPTIASQPLSLRPEACRPAAG | |
| | GAVHTRGLDFACDIYIWAPLAGTCGVLLLSLVITLYCKRGRKKLLYIFK | |
| | QPFMRPVQTTQEEDGCSCRFPEEEEGGCELRVKFSRSADAPAYQQGQNQ | |
| | LYNELNLGRREEYDVLDKRRGRDPEMGGKPRRKNPQEGLYNELQKDKMA | |
| | EAYSEIGMKGERRRGKGHDGLYQGLSTATKDTYDALHMQALPPRGSGEG | |
| | RGSLLTCGDVEENPGPGLTSQLLPPLFFLLACAGNFVHGHKCDITLQEI | |
| | IKTLNSLTEQKTLCTELTVTDIFAASKNTTEKETFCRAATVLRQFYSHH | |
| | EKDTRCLGATAQQFHRHKQLIRFLKRLDRNLWGLAGLNSCPVKEANQST | |
| | LENFLERLKTIMREKYSKCSSSGGGGSGGGGSGGGGSGGGGSGGGSLQI | |
| | YIWAPLAGTCGVLLLSLVITGSGATNFSLLKQAGDVEENPGPGTSLLCW | |
| | MALCLLGADHADAGIHVFILGCFSAGLPKTEANWVNVISDLKKIEDLIQ | |
| | SMHIDATLYTESDVHPSCKVTAMKCFLLELQVISLESGDASIHDTVENL | |
| | IILANNSLSSNGNVTESGCKECEELEEKNIKEFLQSFVHIVQMFINTSR | |
| | ASGGGGSGGGGSGGGGSGGGGSGGGSLQIYIWAPLAGTCGVLLLSLVIT | |
| 400 | ALPVTALLLPLALLLHAARP | Leader (CD8α) |
| 401 | DIQMTQTTSSLSASLGDRVTISCRASQDISKYLNWYQQKPDGTVKLLIY | scFv (CD19) |
| | HTSRLHSGVPSRFSGSGSGTDYSLTISNLEQEDIATYFCQQGNTLPYTF | |
| | GGGTKLEITGGGGSGGGGSGGGGSEVKLQESGPGLVAPSQSLSVTCTVS | |
| | GVSLPDYGVSWIRQPPRKGLEWLGVIWGSETTYYNSALKSRLTIIKDNS | |
| | KSQVFLKMNSLQTDDTAIYYCAKHYYYGGSYAMDYWGQGTSVTVSS | |
| 402 | TTTPAPRPPTPAPTIASQPLSLRPEACRPAAGGAVHTRGLDFACD | Hinge (CD8α) |
| 403 | IYIWAPLAGTCGVLLLSLVITLYC | Transmembrane |
| | | domain (CD8α) |
| 404 | KRGRKKLLYIFKQPFMRPVQTTQEEDGCSCRFPEEEEGGCEL | Costimulatory |
| | | domain (4- |
| | | 1BB) |
| 405 | RVKFSRSADAPAYQQGQNQLYNELNLGRREEYDVLDKRRGRDPEMGGKP | Intracellular |
| | RRKNPQEGLYNELQKDKMAEAYSEIGMKGERRRGKGHDGLYQGLSTATK | signal domain |
| | DTYDALHMQALPPR | (CD3ζ) |
| 406 | ALPVTALLLPLALLLHAARPDIQMTQTTSSLSASLGDRVTISCRASQDI | Complete |
| | SKYLNWYQQKPDGTVKLLIYHTSRLHSGVPSRFSGSGSGTDYSLTISNL | sequence of |
| | EQEDIATYFCQQGNTLPYTFGGGTKLEITGGGGSGGGGSGGGGSEVKLQ | CAR (CD8α |
| | ESGPGLVAPSQSLSVTCTVSGVSLPDYGVSWIRQPPRKGLEWLGVIWGS | Leader + Anti |
| | ETTYYNSALKSRLTIIKDNSKSQVFLKMNSLQTDDTAIYYCAKHYYYGG | CD19-scFv + |
| | SYAMDYWGQGTSVTVSSTTTPAPRPPTPAPTIASQPLSLRPEACRPAAG | CD8α Hinge + |
| | GAVHTRGLDFACDIYIWAPLAGTCGVLLLSLVITLYCKRGRKKLLYIFK | CD8α |
| | QPFMRPVQTTQEEDGCSCRFPEEEEGGCELRVKFSRSADAPAYQQGQNQ | Transmembrane |
| | LYNELNLGRREEYDVLDKRRGRDPEMGGKPRRKNPQEGLYNELQKDKMA | + 4-1BB + |
| | EAYSEIGMKGERRRGKGHDGLYQGLSTATKDTYDALHMQALPPR | CD3ζ) |
| | GSG | linker |
| 301 | EGRGSLLTCGDVEENPGP | T2A peptide |
| 113 | GLTSQLLPPLFFLLACAGNFVHG | Signal (IL-4) |
| 101 | HKCDITLQEIIKTLNSLTEQKTLCTELTVTDIFAASKNTTEKETFCRAA | IL-4 |
| | TVLRQFYSHHEKDTRCLGATAQQFHRHKQLIRFLKRLDRNLWGLAGLNS | |
| | CPVKEANQSTLENFLERLKTIMREKYSKCSS | |
| 506 | SGGGGSGGGGSGGGGSGGGGSGGGSLQ | Lr1 |
| 203 | IYIWAPLAGTCGVLLLSLVIT | transmembrane |
| | | peptide (Ar1) |
| | GSG | linker |
| 300 | ATNFSLLKQAGDVEENPGP | P2A peptide |
| 3 | GTSLLCWMALCLLGADHADA | Signal (TCR) |
| 106 | GIHVFILGCFSAGLPKTEANWVNVISDLKKIEDLIQSMHIDATLYTESD | IL-15 |
| | VHPSCKVTAMKCFLLELQVISLESGDASIHDTVENLIILANNSLSSNGN | |
| | VTESGCKECEELEEKNIKEFLQSFVHIVQMFINTSRA | |
| 506 | SGGGGSGGGGSGGGGSGGGGSGGGSLQ | Lr1 |
| 203 | IYIWAPLAGTCGVLLLSLVIT | transmembrane |
| | | peptide (Ar1) |

Exemplary molecule 10 SP-IL4-Anchoring structure-T2A-SP-IL15-Anchoring structure

| | | |
|---|---|---|
| 532 | GLTSQLLPPLFFLLACAGNFVHGHKCDITLQEIIKTLNSLTEQKTLCTE | SP-IL4- |
| | LTVTDIFAASKNTTEKETFCRAATVLRQFYSHHEKDTRCLGATAQQFHR | Anchoring |
| | HKQLIRFLKRLDRNLWGLAGLNSCPVKEANQSTLENFLERLKTIMREKY | structure-T2A- |
| | SKCSSSGGGGSGGGGSGGGGSGGGGSGGGSLQLENGGTSLSEKTVLLLV | SP-IL15- |
| | TPFLAAAWSLHPGSGEGRGSLLTCGDVEENPGPGTSLLCWMALCLLGAD | Anchoring |
| | HADAGIHVFILGCFSAGLPKTEANWVNVISDLKKIEDLIQSMHIDATLY | structure full |
| | TESDVHPSCKVTAMKCFLLELQVISLESGDASIHDTVENLIILANNSLS | construct |
| | SNGNVTESGCKECEELEEKNIKEFLQSFVHIVQMFINTSRASGGGGSGG | |
| | GGSGGGGSGGGGSGGGSLQLENGGTSLSEKTVLLLVTPFLAAAWSLHP | |
| 113 | GLTSQLLPPLFFLLACAGNFVHG | Signal (IL-4) |
| 101 | HKCDITLQEIIKTLNSLTEQKTLCTELTVTDIFAASKNTTEKETFCRAA | IL-4 |
| | TVLRQFYSHHEKDTRCLGATAQQFHRHKQLIRFLKRLDRNLWGLAGLNS | |
| | CPVKEANQSTLENFLERLKTIMREKYSKCSS | |
| 506 | SGGGGSGGGGSGGGGSGGGGSGGGSLQ | Lr1 |
| 200 | LENGGTSLSEKTVLLLVTPFLAAAWSLHP | GPI-attachment |
| | | signal (CD59) |
| | GSG | Linker |
| 301 | EGRGSLLTCGDVEENPGP | T2A peptide |
| 3 | GTSLLCWMALCLLGADHADA | Signal (TCR) |
| 106 | GIHVFILGCFSAGLPKTEANWVNVISDLKKIEDLIQSMHIDATLYTESD | IL-15 |
| | VHPSCKVTAMKCFLLELQVISLESGDASIHDTVENLIILANNSLSSNGN | |
| | VTESGCKECEELEEKNIKEFLQSFVHIVQMFINTSRA | |
| 506 | SGGGGSGGGGSGGGGSGGGGSGGGSLQ | Lr1 |
| 200 | LENGGTSLSEKTVLLLVTPFLAAAWSLHP | GPI-attachment |
| | | signal (CD59) |

Exemplary molecule 11 SP-IL4-Lr1-Ar1-T2A-SP-IL15-Lr1-Ar1

| | | |
|---|---|---|
| 534 | GLTSQLLPPLFFLLACAGNFVHGHKCDITLQEIIKTLNSLTEQKTLCTE | SP-IL4-Lr1- |
| | LTVTDIFAASKNTTEKETFCRAATVLRQFYSHHEKDTRCLGATAQQFHR | Ar1-T2A-SP- |
| | HKQLIRFLKRLDRNLWGLAGLNSCPVKEANQSTLENFLERLKTIMREKY | IL15-Lr1-Arl |
| | SKCSSSGGGGSGGGGSGGGGSGGGGSGGGSLQIYIWAPLAGTCGVLLLS | full construct |
| | LVITGSGEGRGSLLTCGDVEENPGPGTSLLCWMALCLLGADHADAGIHV | |
| | FILGCFSAGLPKTEANWVNVISDLKKIEDLIQSMHIDATLYTESDVHPS | |
| | CKVTAMKCFLLELQVISLESGDASIHDTVENLIILANNSLSSNGNVTES | |
| | GCKECEELEEKNIKEFLQSFVHIVQMFINTSRASGGGGSGGGGGGGGS | |
| | GGGGSGGGSLQIYIWAPLAGTCGVLLLSLVIT | |
| 113 | GLTSQLLPPLFFLLACAGNFVHG | Signal (IL-4) |
| 101 | HKCDITLQEIIKTLNSLTEQKTLCTELTVTDIFAASKNTTEKETFCRAA | IL-4 |
| | TVLRQFYSHHEKDTRCLGATAQQFHRHKQLIRFLKRLDRNLWGLAGLNS | |
| | CPVKEANQSTLENFLERLKTIMREKYSKCSS | |
| 506 | SGGGGGGGGSGGGGSGGGGSGGGSLQ | Lr1 |
| 203 | IYIWAPLAGTCGVLLLSLVIT | transmembrane |
| | | peptide (Ar1) |
| | GSG | linker |
| 301 | EGRGSLLTCGDVEENPGP | T2A peptide |
| 3 | GTSLLCWMALCLLGADHADA | Signal (TCR) |
| 106 | GIHVFILGCFSAGLPKTEANWVNVISDLKKIEDLIQSMHIDATLYTESD | IL-15 |
| | VHPSCKVTAMKCFLLELQVISLESGDASIHDTVENLIILANNSLSSNGN | |
| | VTESGCKECEELEEKNIKEFLQSFVHIVQMFINTSRA | |
| 506 | SGGGGSGGGGSGGGGSGGGGSGGGSLQ | Lr1 |
| 203 | IYIWAPLAGTCGVLLLSLVIT | transmembrane |
| | | peptide (Ar1) |

### Example B. Lentiviral Vector Preparation

This example illustrates the process of preparing lentiviral vector comprising the various constructs described in Example A, according to some embodiments of the present disclosure.

Plasmid construction and identification: the nucleic acid sequences of the target constructs shown in Table B-1 were synthesized and cloned sequentially into the pK14 vector, and the correct insertion of the targe gene was confirmed by sequencing. Additional constructs shown in Tables A-2, A-3, A-4, A-5, A-6, A-7, A-8, A-9, A-10, and A-11 can be synthetized and tested using the same methods described in Examples B, C, D, and E.

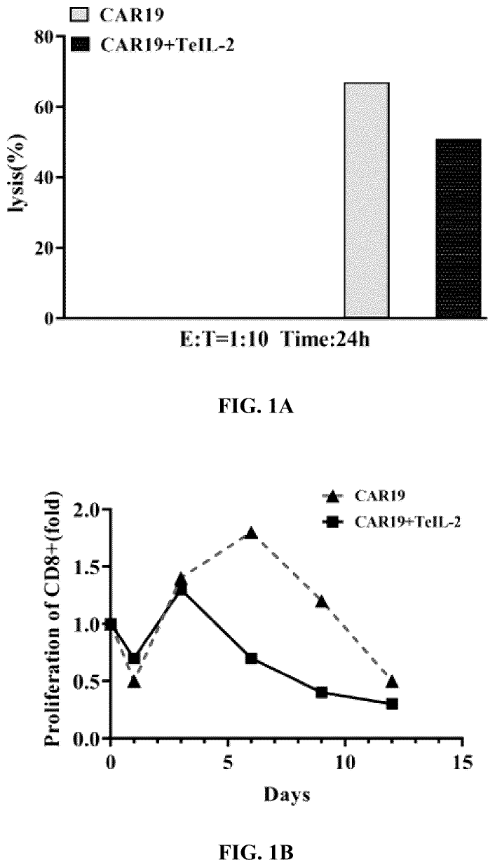

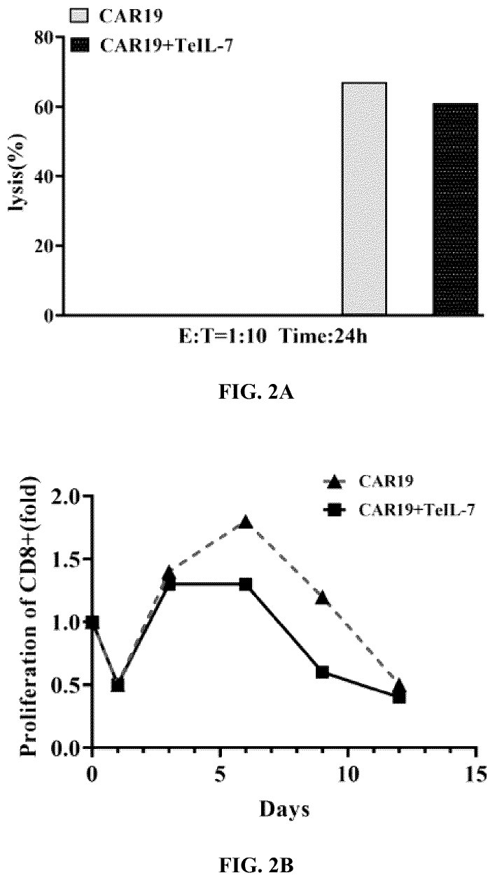

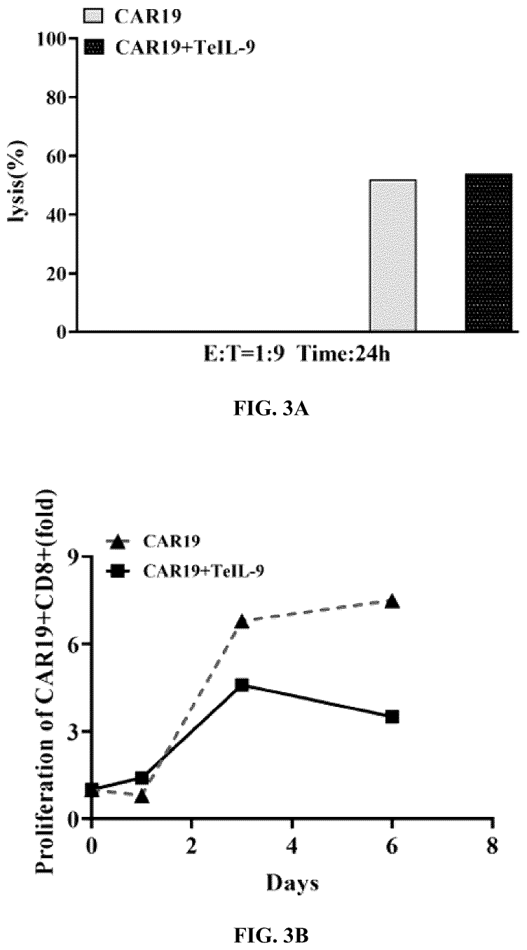

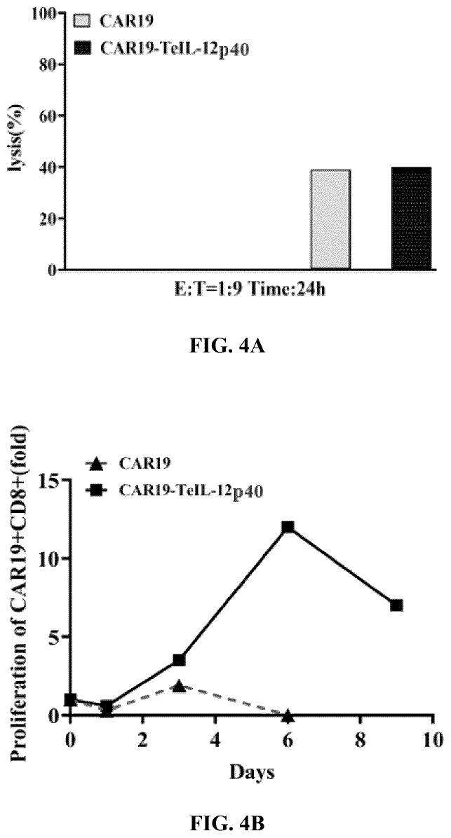

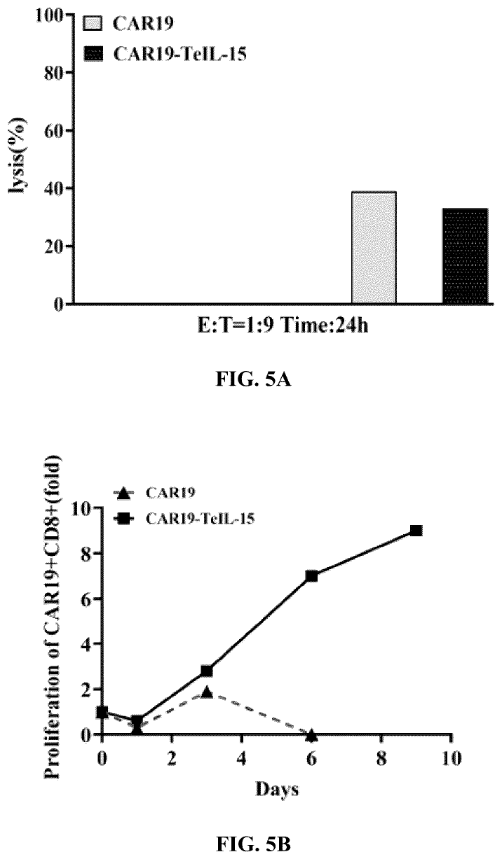

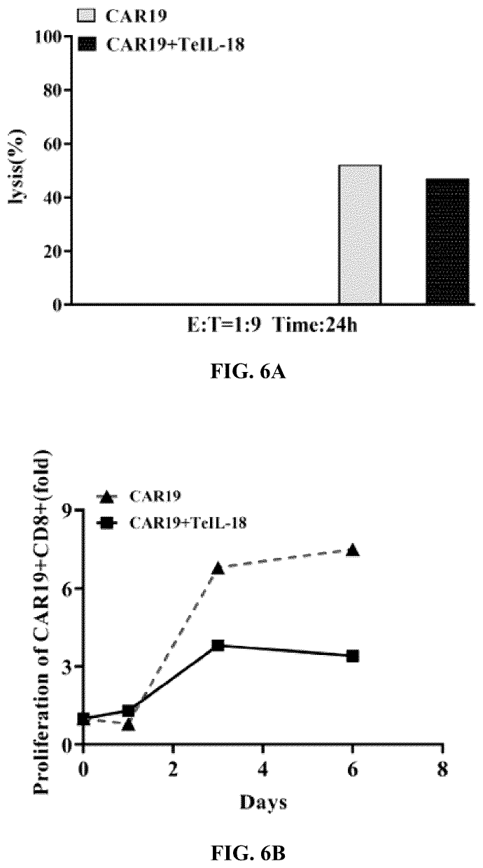

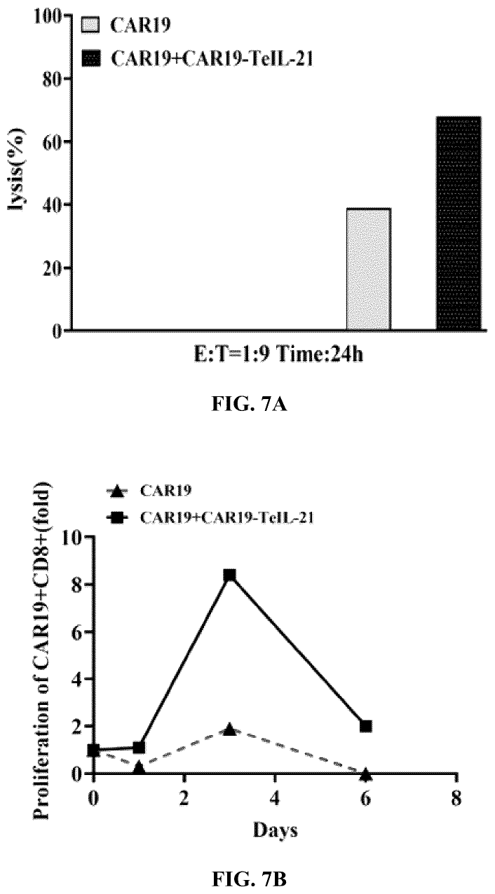

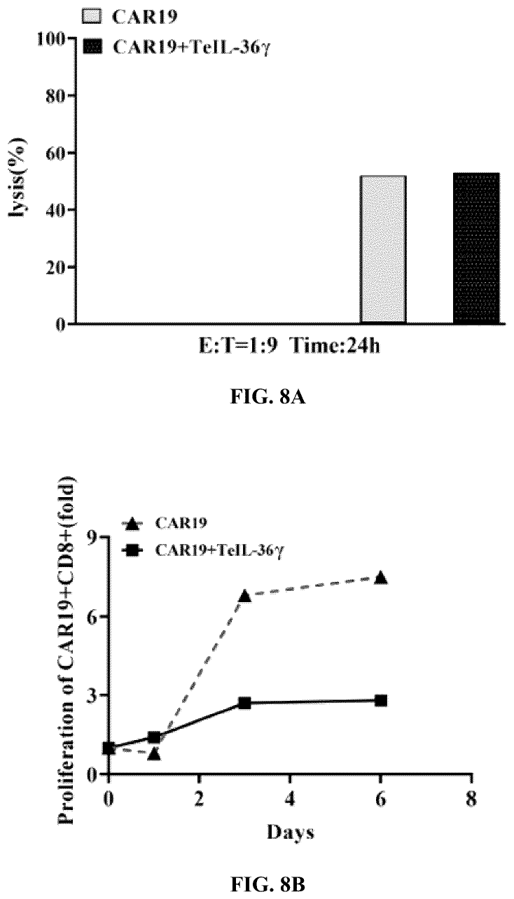

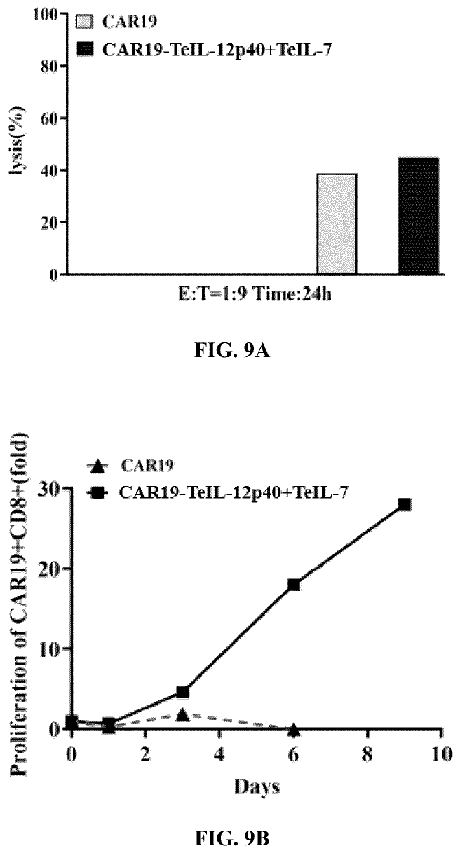

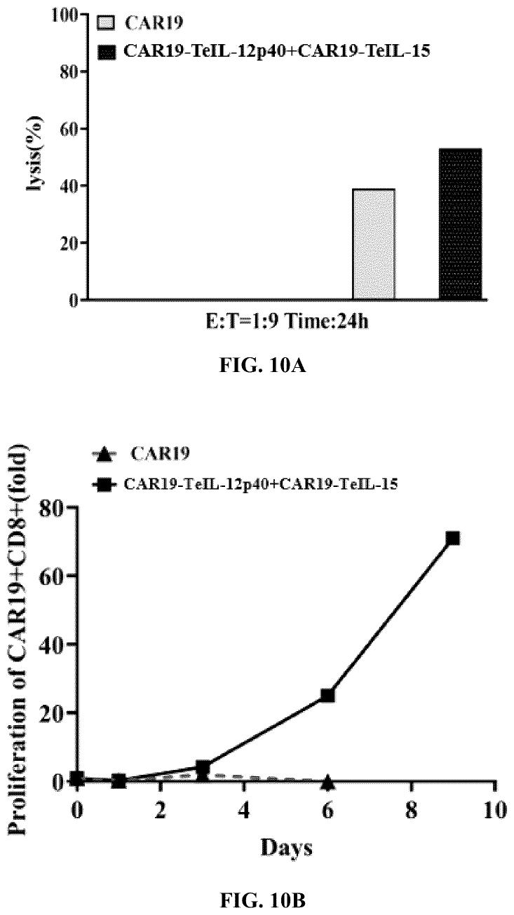

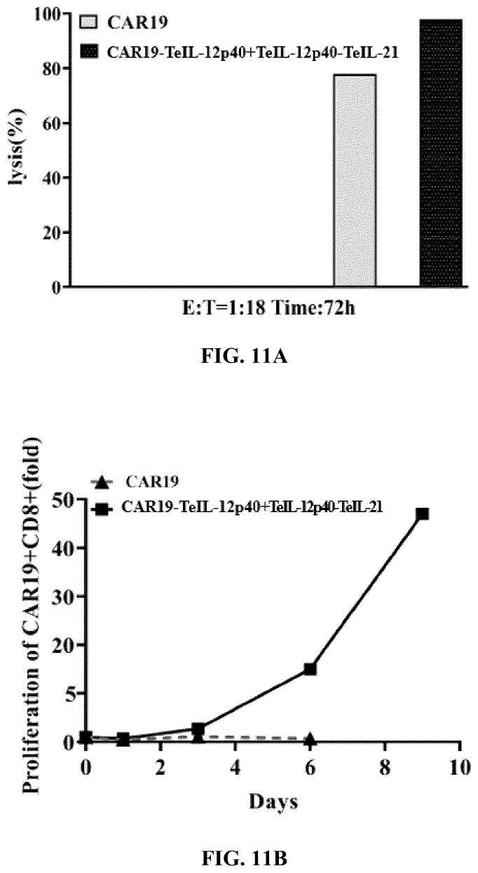

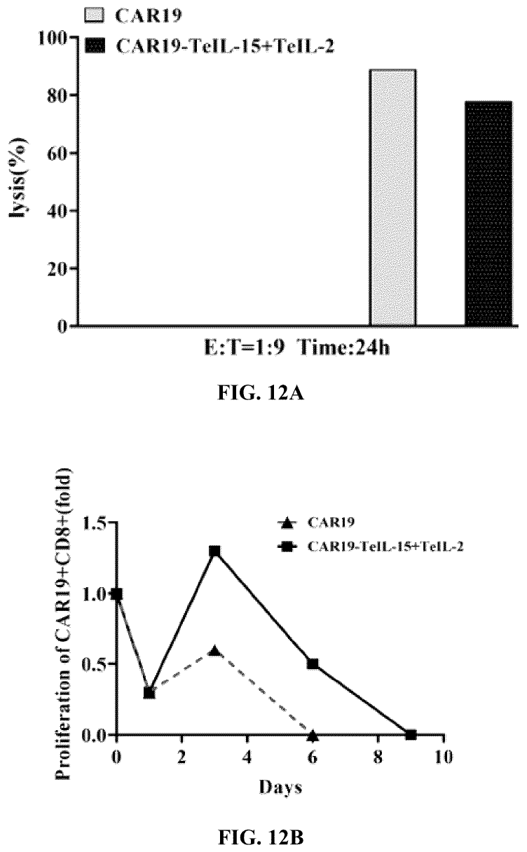

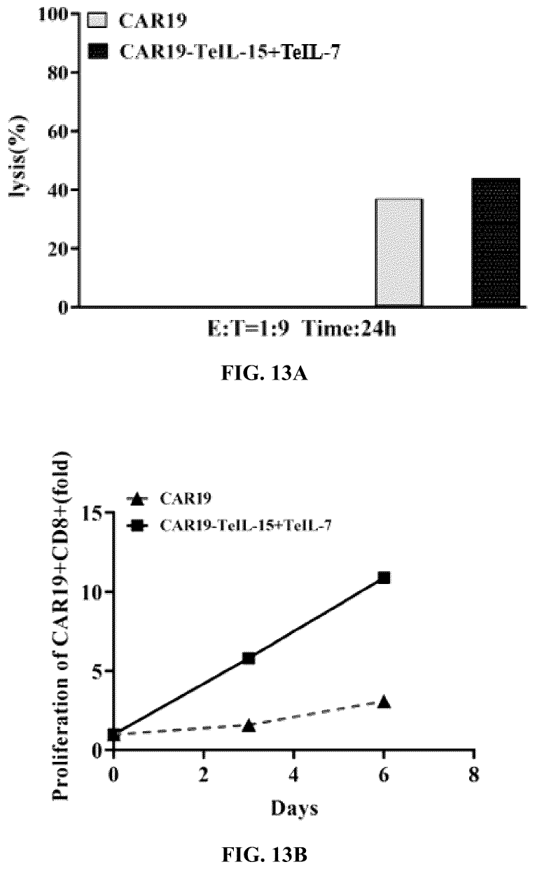

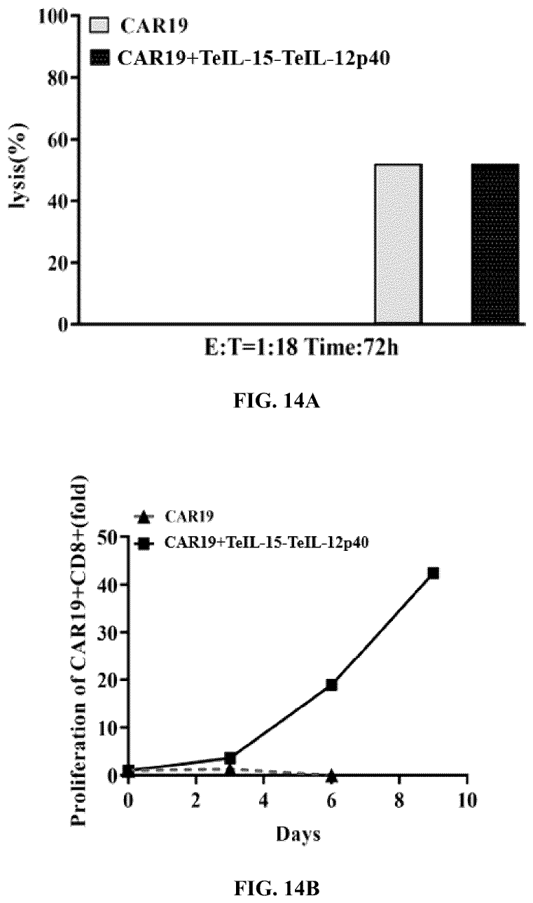

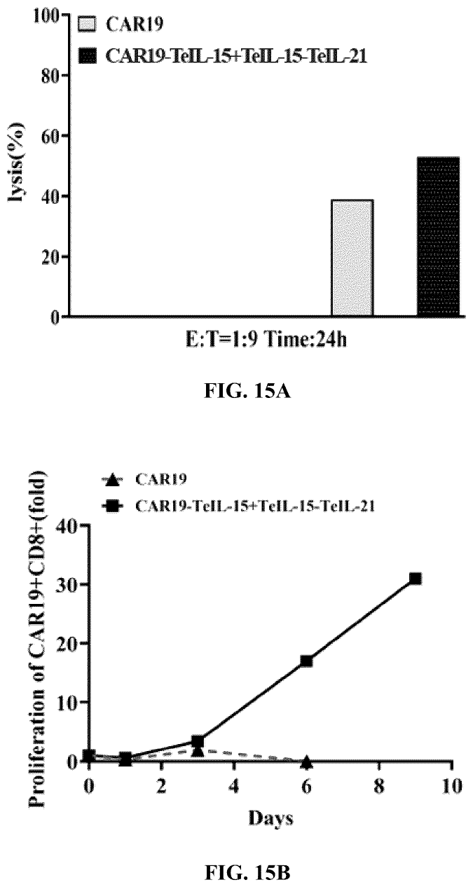

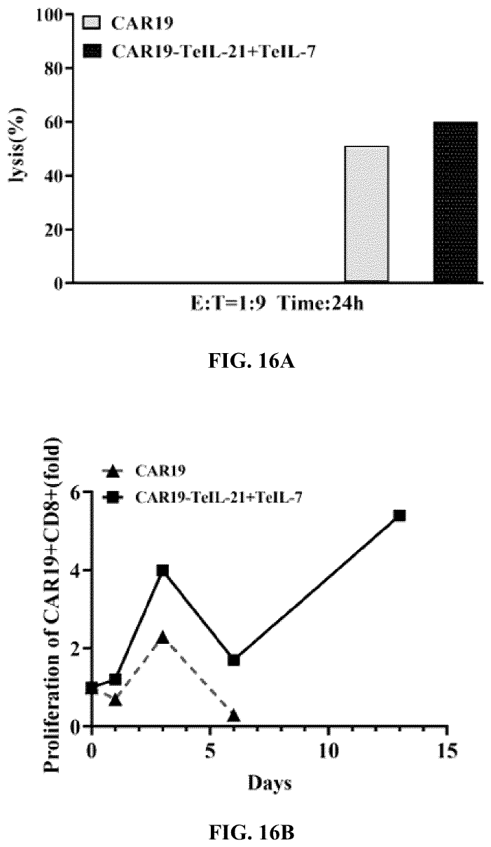

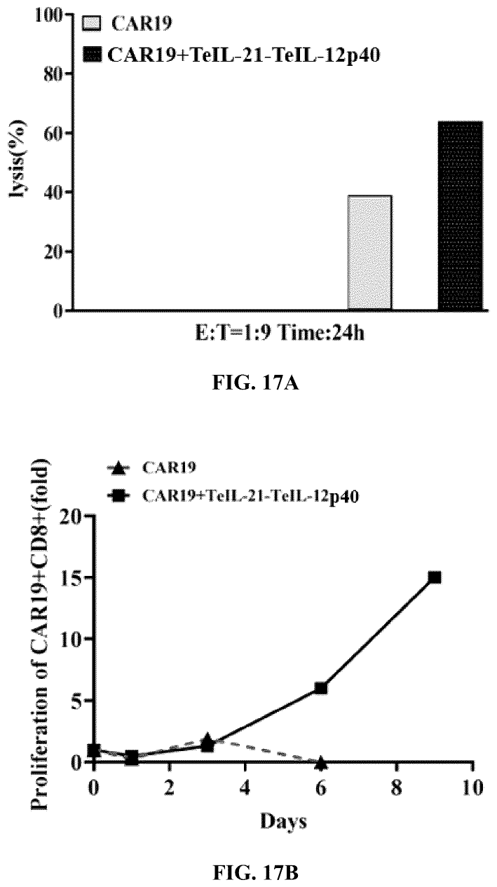

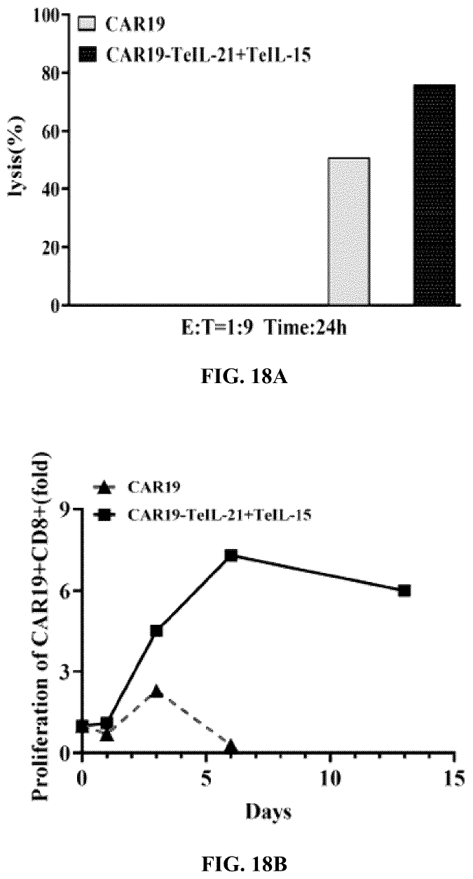

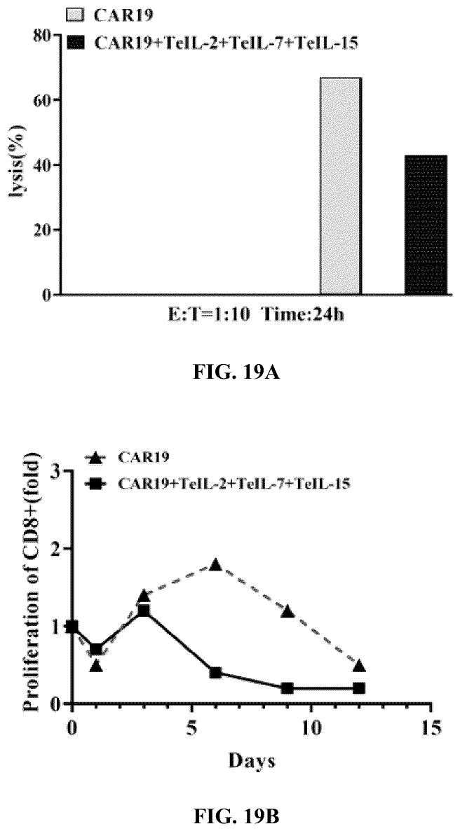

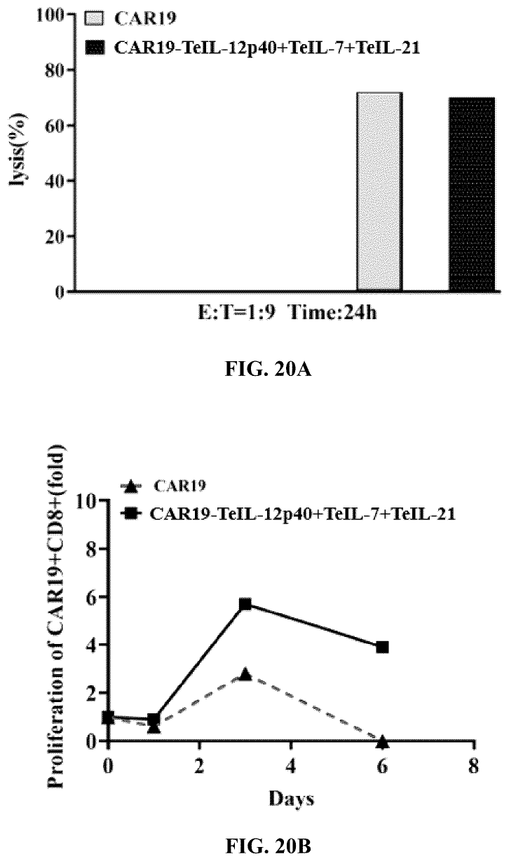

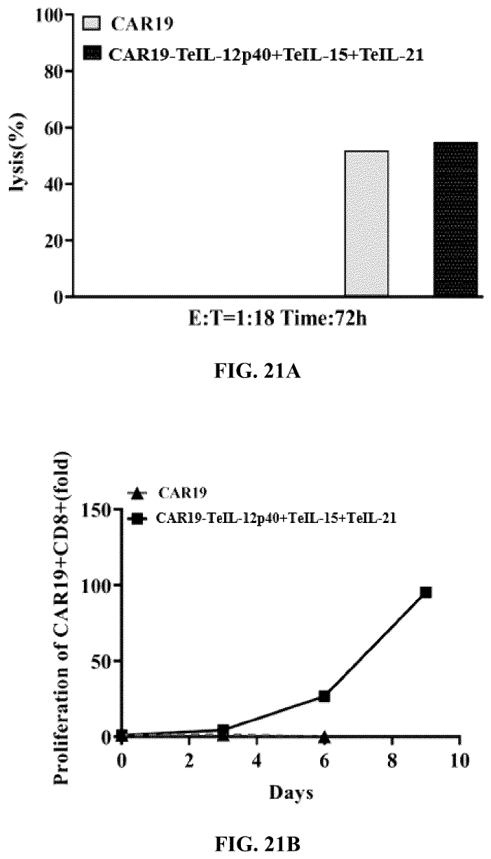

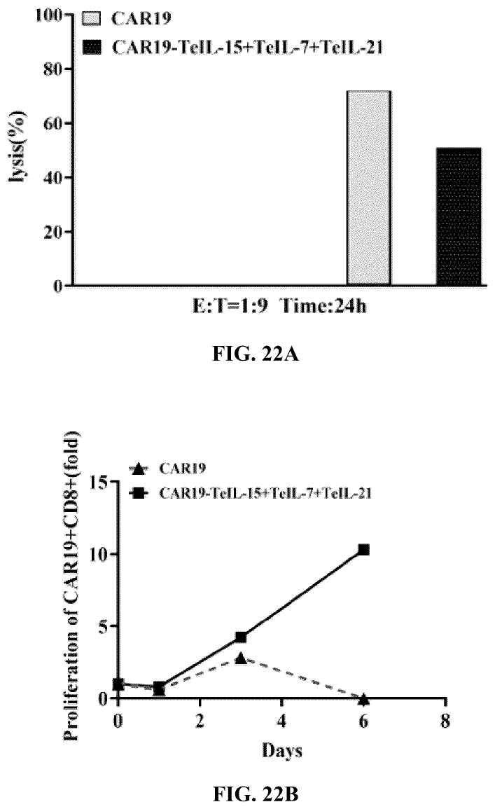

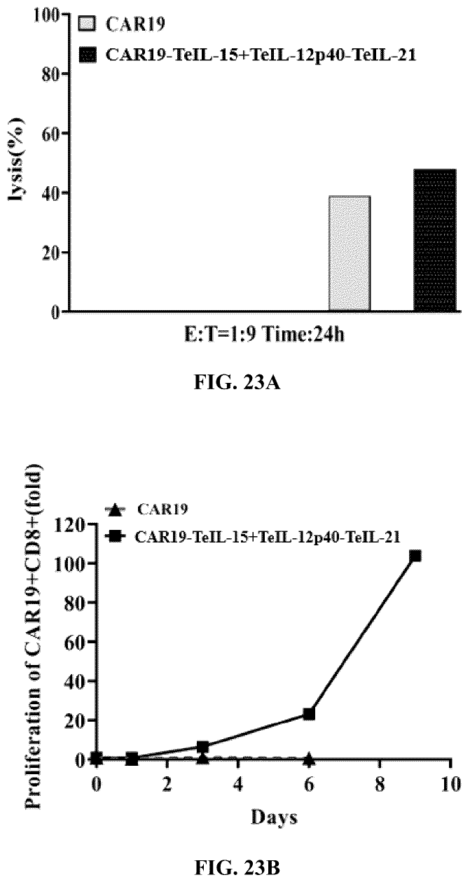

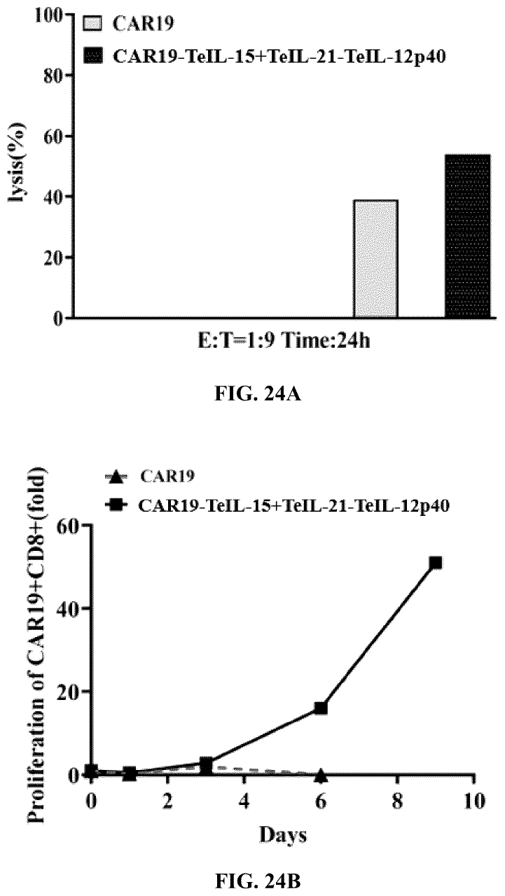

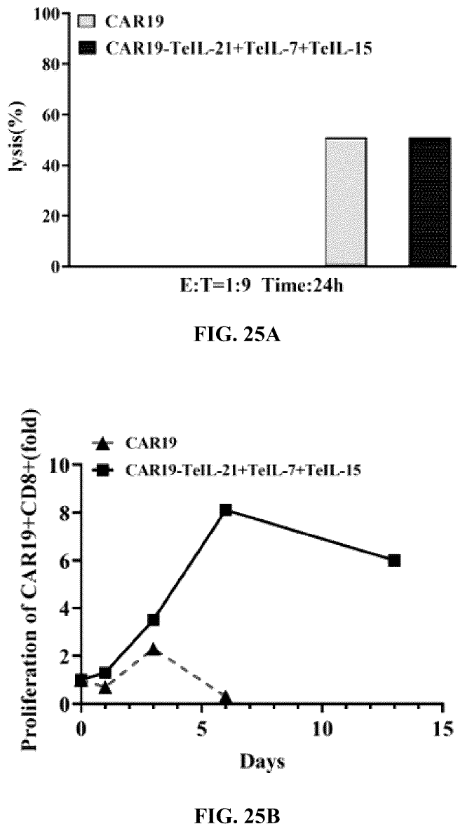

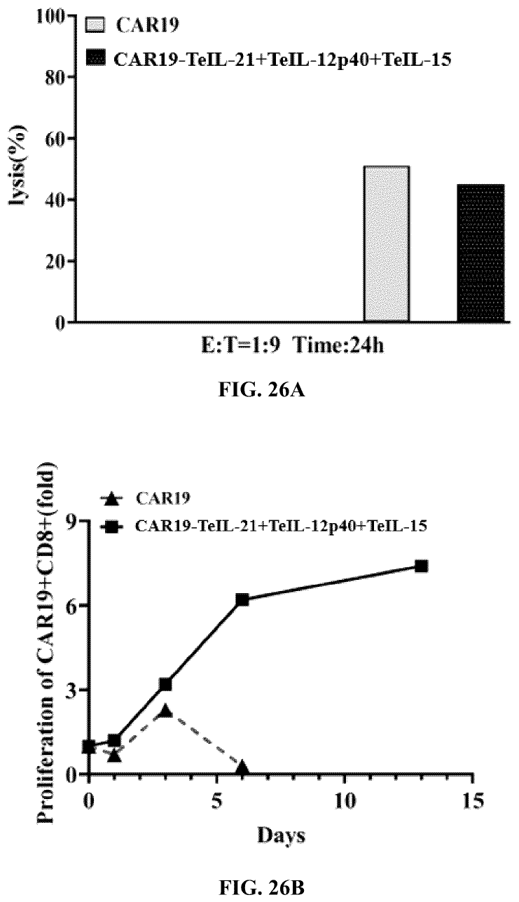

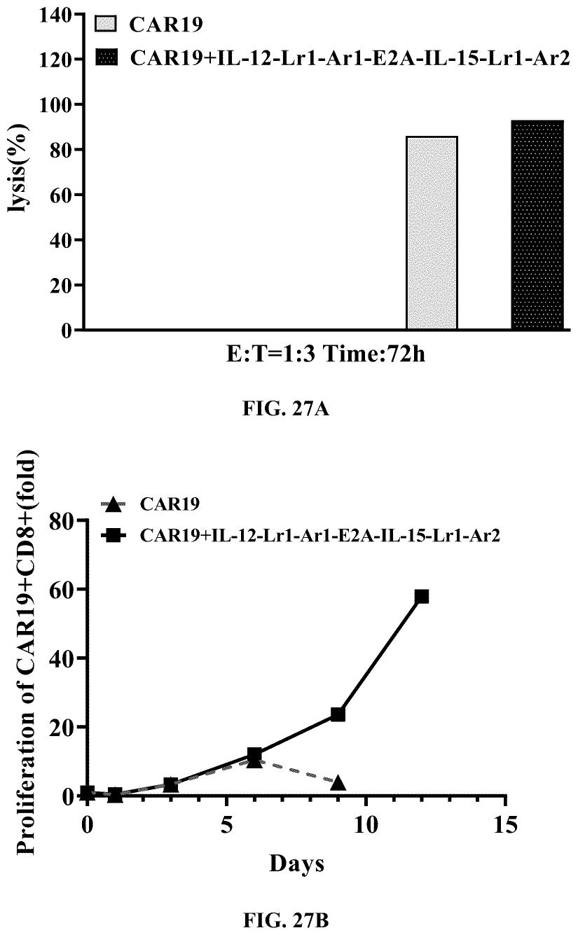

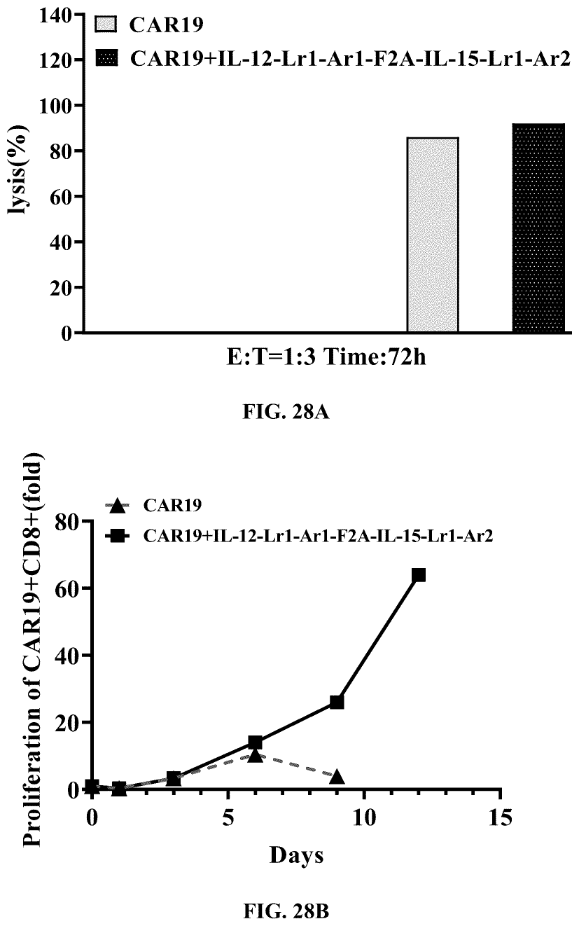

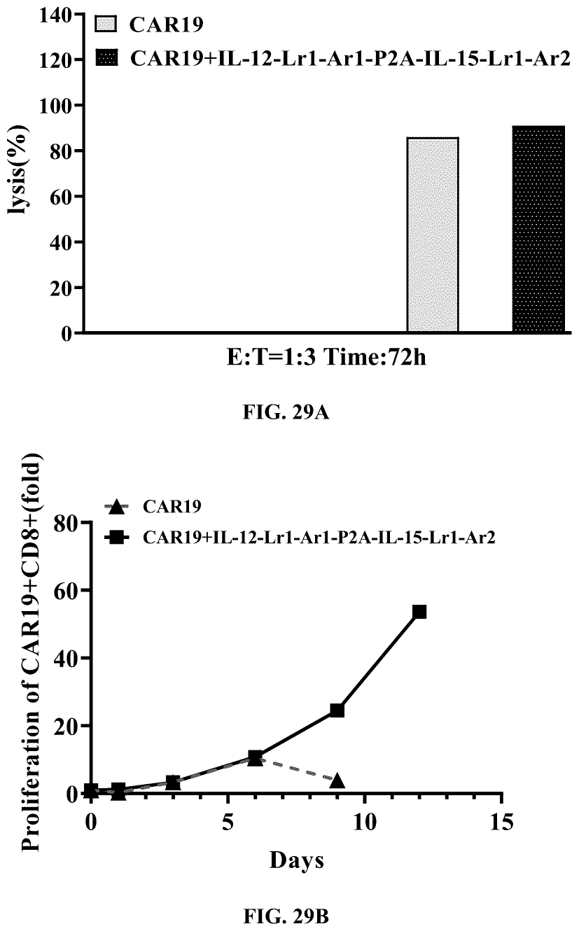

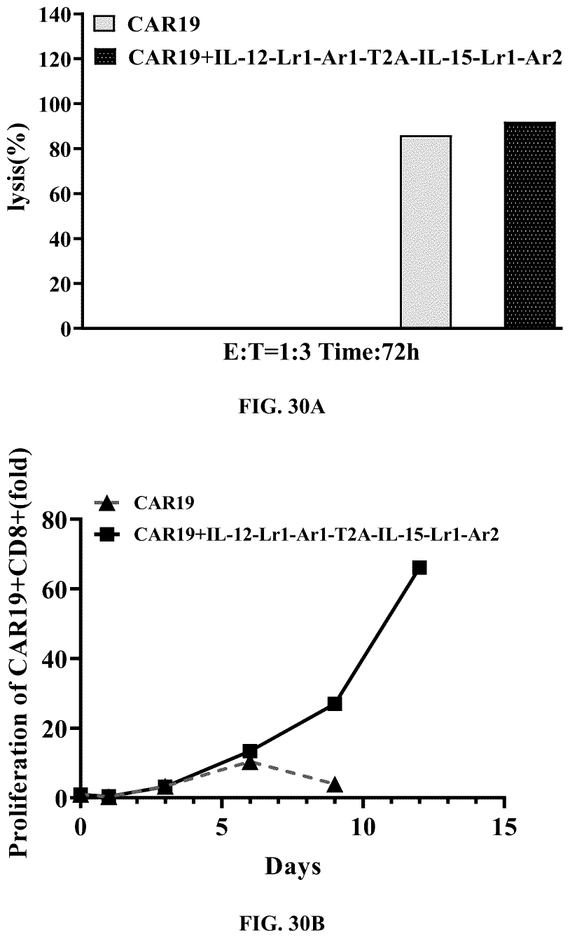

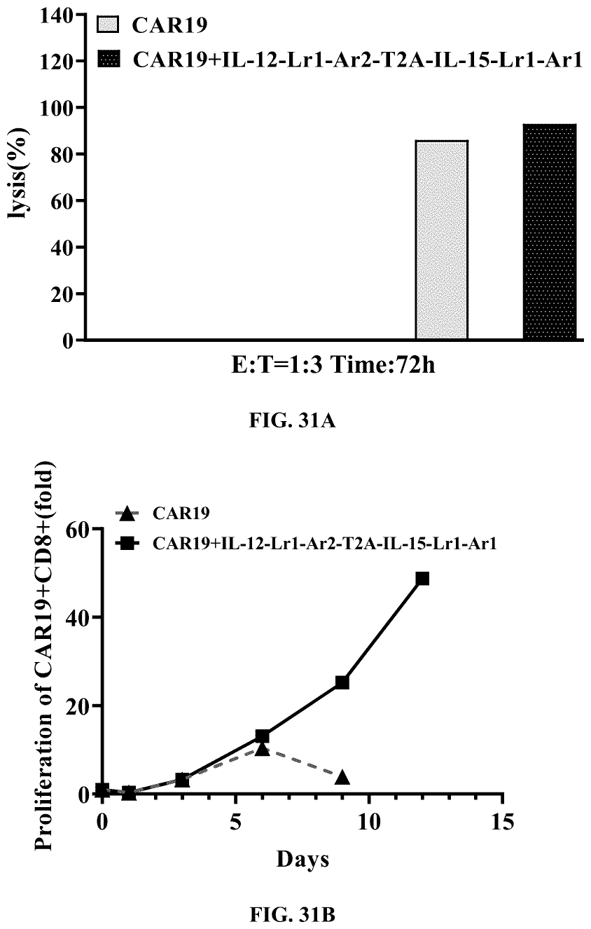

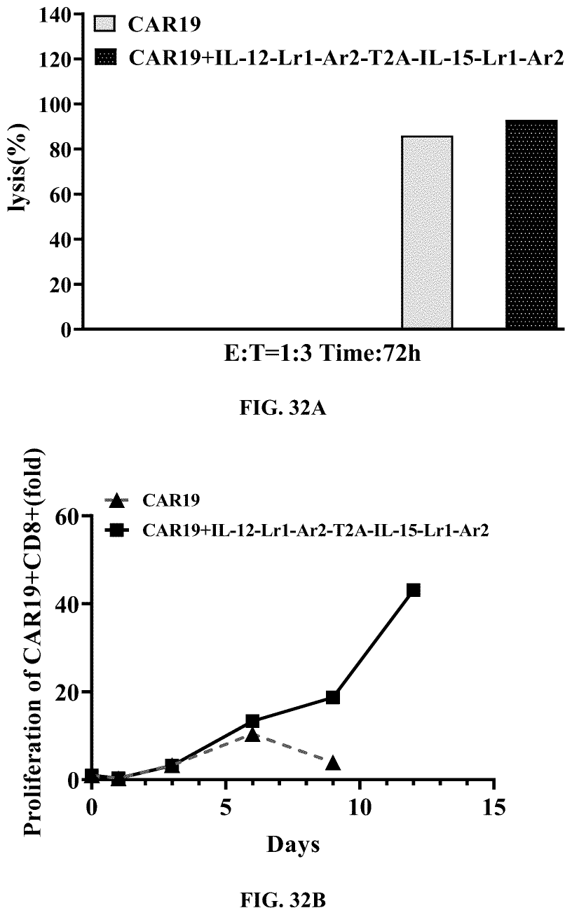

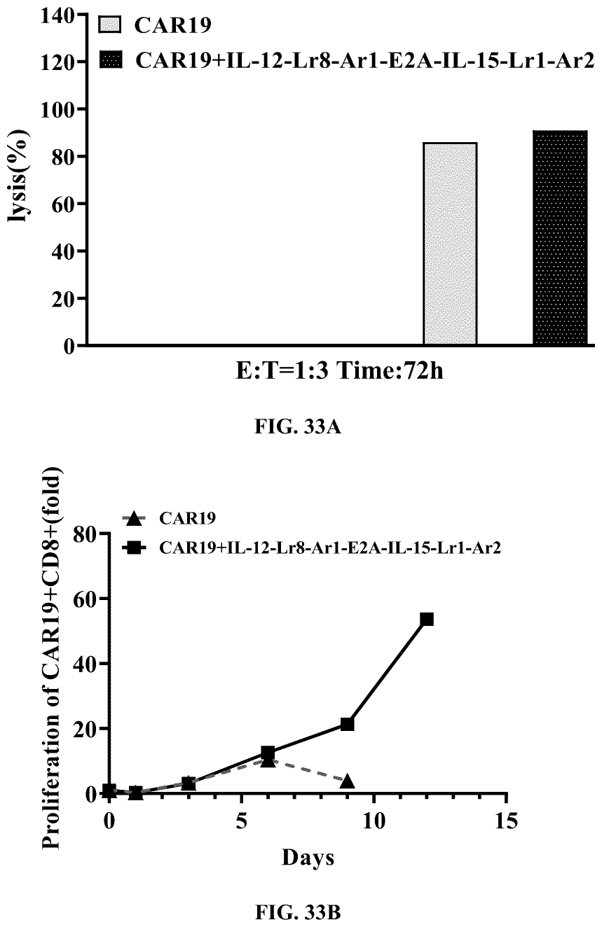

TABLE B-1
Constructs for each group and the corresponding figures showing the testing results

| | | Figures | |
|---|---|---|---|
| | Final expression | showing | Description in the |
| Constructs used | on cell surface | Results | Figures* |
| CAR19 (SEQ ID NO: 456); | CAR19, IL-2 | FIGS. 1A-B | CAR19 + TeIL-2 |
| SP-IL-2-Anchoring structure from Table | | | |
| A-2; | | | |
| CAR19 (SEQ ID NO: 456); | CAR19, IL-7 | FIGS. 2A-B | CAR19 + TeIL-7 |
| SP-IL-7-Anchoring structure from Table | | | |
| A-2; | | | |
| CAR19 (SEQ ID NO: 456); | CAR19, IL-9 | FIGS. 3A-B | CAR19 + TeIL-9 |
| SP-IL-9-Anchoring structure from Table | | | |
| A-2; | | | |
| CAR19-P2A-SP-IL-12p40-Anchoring | CAR19, IL-12p40 | FIGS. 4A-B | CAR19-TeIL-12p40 |
| structure from Table A-5; | | | |
| CAR19-P2A-SP-IL-15-Anchoring | CAR19, IL-15 | FIGS. 5A-B | CAR19-TeIL-15 |
| structure from Table A-5; | | | |
| CAR19 (SEQ ID NO: 456); | CAR19, IL-18 | FIGS. 6A-B | CAR19 + TeIL-18 |
| SP-IL-18-Anchoring structure from Table | | | |
| A-2; | | | |
| CAR19 (SEQ ID NO: 456); | CAR19, IL-21 | FIGS. 7A-B | CAR19 + CAR19-TeIL-21 |
| CAR19-T2A-SP-IL-21-Anchoring | | | |
| structure from Table A-5; | | | |
| CAR19 (SEQ ID NO: 456); | CAR19, IL-36γ | FIGS. 8A-B | CAR19 + TeIL-36γ |
| SP-IL-36γ-Anchoring structure from | | | |
| Table A-2; | | | |
| CAR19-P2A-SP-IL-12p40-Anchoring | CAR19, IL-12p40, | FIGS. 9A-B | CAR19-TeIL- |
| structure from Table A-5; | IL-7 | | 12p40 + TeIL-7 |
| SP-IL-7-Anchoring structure from Table | | | |
| A-2; | | | |
| CAR19-P2A-SP-IL-12p40-Anchoring | CAR19, IL-12p40, | FIGS. 10A-B | CAR19-TeIL- |
| structure from Table A-5; | IL-15 | | 12p40 + CAR19-TeIL-15 |
| CAR19-P2A-SP-IL-15-Anchoring | | | |
| structure from Table A-5; | | | |
| CAR19-P2A-SP-IL-12p40-Anchoring | CAR19, IL-12p40, | FIGS. 11A-B | CAR19-TeIL- |
| structure from Table A-5; | IL-21 | | 12p40 + TeIL-12p40-TeIL- |
| SP1-IL-12p40-Anchoring structure 1- | | | 21 |
| T2A-SP2-IL-21-Anchoring structure 2 | | | |
| from Table A-3; | | | |
| CAR19-P2A-SP-IL-15-Anchoring | CAR19, IL-15, IL- | FIGS. 12A-B | CAR19-TeIL-15 + TeIL-2 |
| structure from Table A-5; | 2 | | |
| SP-IL-2-Anchoring structure from Table | | | |
| A-2; | | | |
| CAR19-P2A-SP-IL-15-Anchoring | CAR19, IL-15, IL- | FIGS. 13A-B | CAR19-TeIL-15 + TeIL-7 |
| structure from Table A-5; | 7 | | |
| SP-IL-7-Anchoring structure from Table | | | |
| A-2; | | | |
| CAR19 (SEQ ID NO: 456); | CAR19, IL-15, IL- | FIGS. 14A-B | CAR19 + TeIL-15-TeIL- |
| SP1-IL-15-Anchoring structure 1-P2A- | 12p40 | | 12p40 |
| SP2-IL-12p40-Anchoring structure 2 | | | |
| from Table A-3; | | | |
| CAR19-P2A-SP-IL-15-Anchoring | CAR19, IL-15, IL- | FIGS. 15A-B | CAR19-TeIL-15 + TeIL-15- |
| structure from Table A-5; | 21 | | TeIL-21 |
| SP1-IL-15-Anchoring structure 1-P2A- | | | |
| SP2-IL-21-Anchoring structure 2 from | | | |
| Table A-3; | | | |
| CAR19-T2A-SP-IL-21-Anchoring | CAR19, IL-21, IL- | FIGS. 16A-B | CAR19-TeIL-21 + TeIL-7 |
| structure from Table A-5; | 7 | | |
| SP-IL-7-Anchoring structure from Table | | | |
| A-2; | | | |
| CAR19 (SEQ ID NO: 456); | CAR19, IL-21, IL- | FIGS. 17A-B | CAR19 + TeIL-21-TeIL- |
| SP1-IL-21-Anchoring structure 1-T2A- | 12p40 | | 12p40 |
| SP2-IL-12p40-Anchoring structure 2 | | | |
| from Table A-3; | | | |
| CAR19-T2A-SP-IL-21-Anchoring | CAR19, IL-21, IL- | FIGS. 18A-B | CAR19-TeIL-21 + TeIL-15 |
| structure from Table A-5; | 15 | | |
| SP-IL-15-Anchoring structure from Table | | | |
| A-2; | | | |
| CAR19 (SEQ ID NO: 456); | CAR19, IL-2, IL- | FIGS. 19A-B | CAR19 + TeIL-2 + TeIL- |
| SP-IL-2-Anchoring structure from Table | 7, IL-15 | | 7 + TeIL-15 |
| A-2; | | | |
| SP-IL-7-Anchoring structure from Table | | | |
| A-2; | | | |
| SP-IL-15-Anchoring structure from Table | | | |
| A-2; | | | |
| CAR19-P2A-SP-IL-12p40-Anchoring | CAR19, IL-12p40, | FIGS. 20A-B | CAR19-TeIL- |
| structure from Table A-5; | IL-7, IL-21 | | 12p40 + TeIL-7 + TeIL-21 |
| SP-IL-7-Anchoring structure from Table | | | |
| A-2; | | | |
| SP-IL-21-Anchoring structure from Table | | | |
| A-2; | | | |
| CAR19-P2A-SP-IL-12p40-Anchoring | CAR19, IL-12p40, | FIGS. 21A-B | CAR19-TeIL- |
| structure from Table A-5; | IL-15, IL-21 | | 12p40 + TeIL-15 + TeIL-21 |
| SP-IL-15-Anchoring structure from Table | | | |
| A-2; SP-IL-21-Anchoring structure from Table | | | |
| A-2; | | | |
| CAR19-P2A-SP-IL-15-Anchoring | CAR19, IL-15, IL- | FIGS. 22A-B | CAR19-TeIL-15 + TeIL- |
| structure from Table A-5; | 7, IL-21 | | 7 + TeIL-21 |
| SP-IL-7-Anchoring structure from Table | | | |
| A-2; | | | |
| SP-IL-21-Anchoring structure from Table | | | |
| A-2; | | | |
| CAR19-P2A-SP-IL-15-Anchoring | CAR19, IL-15, IL- | FIGS. 23A-B | CAR19-TeIL-15 + TeIL- |
| structure from Table A-5; | 12p40, IL-21 | | 12p40-TeIL-21 |
| SP1-IL-12p40-Anchoring structure 1- | | | |
| T2A-SP2-IL-21-Anchoring structure 2 | | | |
| from Table A-3; | | | |
| CAR19-P2A-SP-IL-15-Anchoring | CAR19, IL-15, IL- | FIGS. 24A-B | CAR19-TeIL-15 + TeIL-21- |
| structure from Table A-5; | 12p40, IL-21 | | TeIL-12p40 |
| SP1-IL-21-Anchoring structure 1-T2A- | | | |
| SP2-IL-12p40-Anchoring structure 2 | | | |
| from Table A-3; | | | |
| CAR19-T2A-SP-IL-21-Anchoring | CAR19, IL-15, IL- | FIGS. 25A-B | CAR19-TeIL-21 + TeIL- |
| structure from Table A-5; | 7, IL-21 | | 7 + TeIL-15 |
| SP-IL-7-Anchoring structure from Table | | | |
| A-2; | | | |
| SP-IL-15-Anchoring structure from Table | | | |
| A-2; | | | |
| CAR19-T2A-SP-IL-21-Anchoring | CAR19, IL-15, IL- | FIGS. 26A-B | CAR19-TeIL-21 + TeIL- |
| structure from Table A-5; | 12p40, IL-21 | | 12p40 + TeIL-15 |
| SP-IL-12p40-Anchoring structure from | | | |
| Table A-2; | | | |
| SP-IL-15-Anchoring structure from Table | | | |
| A-2; | | | |
| CAR19 (SEQ ID NO: 456); | CAR 19, IL- | FIGS. 27A-B | CAR19 + IL-12-Lr1-Ar1- |
| IL-12p40-Lr1-Ar1-E2A-IL-15-Lr1-Ar2 | 12p40, IL-15 | | E2A-IL-15-Lr1-Ar2 |
| from Table A-3; | | | |
| CAR19 (SEQ ID NO: 456); | CAR 19, IL- | FIGS. 28A-B | CAR19 + IL-12-Lr1-Ar1- |
| IL-12p40-Lr1-Ar1-F2A-IL-15-Lr1-Ar2 | 12p40, IL-15 | | F2A-IL-15-Lr1-Ar2 |
| from Table A-3; | | | |
| CAR19 (SEQ ID NO: 456); | CAR 19, IL- | FIGS. 29A-B | CAR19 + IL-12-Lr1-Ar1- |
| IL-12p40-Lr1-Ar1-P2A-IL-15-Lr1-Ar2 | 12p40, IL-15 | | P2A-IL-15-Lr1-Ar2 |
| from Table A-3; | | | |
| CAR19 (SEQ ID NO: 456); | CAR 19, IL- | FIGS. 30A-B | CAR19 + IL-12-Lr1-Ar1- |
| IL-12p40-Lr1-Ar1-T2A-IL-15-Lr1-Ar2 | 12p40, IL-15 | | T2A-IL-15-Lr1-Ar2 |
| from Table A-3; | | | |
| CAR19 (SEQ ID NO: 456); | CAR 19, IL- | FIGS. 31A-B | CAR19 + IL-12-Lr1-Ar2- |
| IL-12p40-Lr1-Ar2-T2A-IL-15-Lr1-Ar1 | 12p40, IL-15 | | T2A-IL-15-Lr1-Ar1 |
| from Table A-3; | | | |
| CAR19 (SEQ ID NO: 456); | CAR 19, IL- | FIGS. 32A-B | CAR19 + IL-12-Lr1-Ar2- |
| IL-12p40-Lr1-Ar2-T2A-IL-15-Lr1-Ar2 | 12p40, IL-15 | | T2A-IL-15-Lr1-Ar2 |
| from Table A-3; | | | |
| CAR19 (SEQ ID NO: 456); | CAR 19, IL- | FIGS. 33A-B | CAR19 + IL-12-Lr8-Ar1- |
| IL-12p40-Lr8-Ar1-E2A-IL-15-Lr1-Ar2 | 12p40, IL-15 | | E2A-IL-15-Lr1-Ar2 |
| from Table A-3; | | | |

*Unless specified otherwise, the symbol “+” when used in the context of describing nucleic acid constructs means the constructs are on different vectors;
unless specified otherwise, the term “IL-12” when used in the figures, figure legends, or figure description refers to “IL-12p40.”

Lentivirus packaging: 5 ml Optim-MEM was added into a sterile 15 ml centrifuge tube, the viral packaging vector and viral envelope vector were added according to the ratio of pK14-CD19:pLP1:pLP2:pLP-VSVG=5:4:3:1. Then. 800 μL PEI transfection reagent was added and mixed immediately, followed by incubation at room temperature for 15 min. After that, the plasmid/vector/transfection reagent complex was added dropwise into the culture flask of 293T cells. Virus supernatants were collected at 24 h and 48 h, combined, and ultracentrifuged (25000 g. 4° C., 3 h) to obtain concentrated lentivirus.

The general preparation method of the vector: 5 ml Optim-MEM was added into a sterile 15 ml centrifuge tube, then the plasmid was added according to the ratio of lentiviral recombinant vector carrying the target gene: pLP1:pLP2:pLP-VSVG=5:4:3:1. Then. 800 ul PEI transfection reagent was added and mix immediately, followed by incubation at room temperature for 15 min. After that, the plasmid/vector/transfection reagent complex was added dropwise into the culture flask of 293T cells. Virus supernatants were collected at 24 h and 48 h, combined, and ultracentrifuged (25000 g. 4° C. 3 h) to obtain concentrated lentivirus.

### Example C. T Cell Transduction and Culturing

This example illustrates the process of transducing and culturing T cells by the lentiviral vector described in Example B, according to some embodiments of the present disclosure.

The cryopreserved human umbilical cord blood was thawed, and the cells were cultured at a density of about 1×10⁶/mL in 1640 medium (HyClone, Cytiva). T cells were stimulated by magnetic beads with anti-CD3 and CD28 antibodies (Miltenyi biotec, Germany). Magnetic beads and T cells were mixed at a ratio of 1:1. The recombinant human IL-7 (Acrobiosystems. Beijing) and recombinant human IL-15 (Acrobiosystems. Beijing) were added to the medium to reach a final concentration of 10 ng/mL. The medium was incubated at 37° C. 5% CO₂ for 48 hours, then T cells were sorted by CD4/CD8 magnetic beads.

The CD8+ T cells were obtained after sorting and were adjusted to about 2×10⁵/mL density. The concentrated lentivirus prepared in Example B was added to the CD8+ T cells in an amount of MOI=25. Then, the cells were cultured in 1640 medium (Hyclone. Cytiva) supplemented with recombinant human IL-7 at a final concentration of 10 ng/mL, recombinant human IL-15 at a final concentration of 10 ng/ml, recombinant IL-2 (Quankang, Shangdong) at a final concentration of 200 IU/mL. L-glutamine (Gibco, US) at a final concentration of 2 mM, 2-mercaptoethanol (Gibco, US) at a final concentration of 55 μM. The cells were cultured for 48 h, and the engineered T cells expressing the target construct were obtained. Subsequent cell media replenishment was performed every 2-3 days at a cell density of about 4×10⁵/mL.

When the virus-infected T cells were cultured for 5 days, the T cell construct expression was evaluated. The anti-scFv antibody labeled with Fluorescein Isothiocyanate (FITC) was used to detect CAR19 (Acrobiosystems. Beijing), the anti-IL-12 antibody conjugated to Phycoerythrin (PE) detects IL-12 (Thermo, US), and labeled IL-15 antibody conjugated to Allophycocyanin (APC) detects IL-15 (R&D, Minnesota, MN). Data were acquired using a flow cytometer (Agilent. California) and analyzed with its own software (NovoExpress).

The CD4+ T cells were obtained after sorting and were adjusted to about 2×10\hat{ }5/mL density. The concentrated lentivirus prepared in Example B was added to the CD4+ CAR-T cells in an amount of MOI=25. Then, the cells were cultured in 1640 medium (Hyclone. Cytiva) supplemented with recombinant human IL-7 at a final concentration of 10 ng/mL, recombinant human IL-15 at a final concentration of 10 ng/mL, recombinant IL-2 (Quankang. Shangdong) at a final concentration of 200 IU/mL. L-glutamine (Gibco, US) at a final concentration of 2 mM, 2-mercaptoethanol (Gibco, US) at a final concentration of 55 μM. The cells were cultured for 48 h, and the engineered T cells expressing the target construct were obtained. Subsequent cell media replenishment was performed every 2-3 days at a cell density of about 4×10\hat{ }5/mL

The final expression of CAR and cytokines on membranes of T cells are summarized in Table A-1.

### Example D. T Cell Cytotoxicity Assessment

This example illustrates the process of evaluating the cytotoxicity of the engineered T cells described in Example C, according to some embodiments of the present disclosure.

CAR19-T cells (control) or T cells expressing target constructs were mixed with tumor cells expressing Enhanced Green Fluorescent Protein (EGFP) at a ratio of 1:9 (or otherwise stated in the Figures as E:T ratio), respectively, and then added to a 24-well plate. The plates were placed in a 37° C. 5% CO₂ incubator for co-cultivation for 24 h and 72 h. The tumor cells were selected from Nalm6-EGFP, Nalmawa-EGFP or other B cell line tumors. The ability of T cells to recognize and kill hematological tumor cells was assessed and compared in vitro. Cytotoxicity was evaluated based on the number of tumor cells that were alive as compared to the initial number of tumor cells before T cells were added. The data of each group are shown in the bar charts as illustrated in FIGS. 1A, 2A, 3A, 4A, 5A, 6A, 7A, 8A, 9A, 10A, 11A, 12A, 13A, 14A, 15A, 16A, 17A, 18A, 19A, 20A, 21A, 22A, 23A, 24A, 25A, 26A, 27A, 28A, 29A, 30A, 31A, 32A, 33A, 34, and 37. E:T ratio refers to Effector to Target ratio, which is the ratio of Engineered T cells (either CAR-19 control group or target-expressing group) to tumor cells.

### Example E. T Cell Proliferation Assessment

This example illustrates the process of evaluating the proliferation and viability of the engineered T cells described in Example C, according to some embodiments of the present disclosure.

To evaluate the ability of T cells to continuously expand in vitro after killing the tumor cells. CAR19-T cells (control) or T cells expressing target construct was mixed with tumor cells in a 1:9 effector-to-tumor ratio, respectively, and was added in a 24-well plate. Conjugated APC-labeled anti-CD8 antibody and FITC-labeled anti-scFv antibody were used for simultaneous staining and T cells were detected on flow cytometry (day 0) then on the third day, and every three days thereafter. Cells were maintained at a density of 4×10⁵/mL until T cells are dead. The cumulative proliferation folds were calculated based on (the number of cells on day n)/(number of cells on day 0). The data of each group are shown in the line graphs as illustrated in FIGS. 1B, 2B, 3B, 4B, 5B, 6B, 7B, 8B, 9B, 10B, 11B, 12B, 13B, 14B, 15B, 16B, 17B, 18B, 19B, 20B, 21B, 22B, 23B, 24B, 25B, 26B, 27B, 28B, 29B, 30B, 31B, 32B, 33B, 34A, 34B, 35A, 35B, 36A, 36B, 38A, and 38B.

### Example F. T Cell Cytotoxicity Assessment

This example illustrates the process of evaluating the cytotoxicity of the engineered CAR-T cells (CAR19+TeIL-4) comprising constructs as described in Table A-8 and A-9. The engineered CAR-T cells were produced using the method described in Examples B and C.

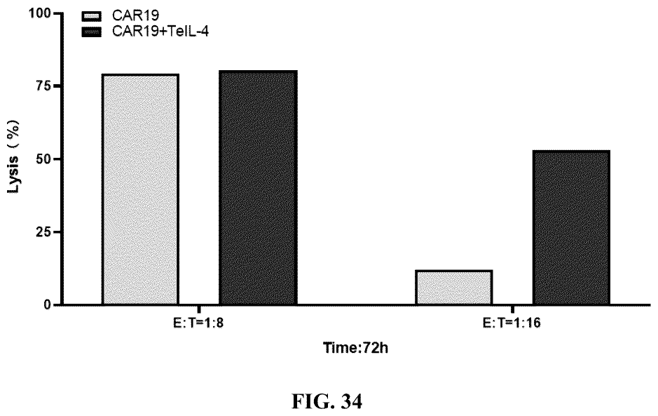

CAR19 T cells (control group. CAR19) or CAR-T cells expressing target constructs (CAR19+TeIL-4) were mixed with tumor cells expressing Enhanced Green Fluorescent Protein (EGFP) at a ratio of 1:8 or 1:16 (as stated in the Figures as E:T ratio), respectively, and then added to a 48-well plate. The plates were placed in a 37° C. 5% CO₂ incubator for co-cultivation for 72 h. Nalm6-EGFP tumor cells were used. The ability of CAR-T cells to recognize and kill hematological tumor cells was assessed and compared in vitro. Cytotoxicity was evaluated based on the number of tumor cells that were alive as compared to the initial number of tumor cells before CAR-T cells were added. Percentage of cell lysis are shown in the bar charts as illustrated in FIG. 34. E:T ratio refers to Effector to Target ratio, which is the ratio of Engineered T cells (either CAR-19 control group or target-expressing group) to tumor cells.

CAR19+TeIL-4 group at E:T ratio of 1:16 showed significantly higher percentage of cell lysis (˜50%) as compared to the CAR19 T cells control group (˜12%) after the 72 h co-cultivation. As such, the CAR19+TeIL-4 group demonstrated higher ability of tumor killing as compared to the CAR19 T cells control group.

### Example G. T Cell Proliferation Assessment

This example illustrates the process of evaluating the proliferation and viability of the engineered T cells (CAR19+TeIL-4) comprising constructs as described in Table A-8 and A-9. The engineered CAR-T cells were produced using the method described in Examples B and C.

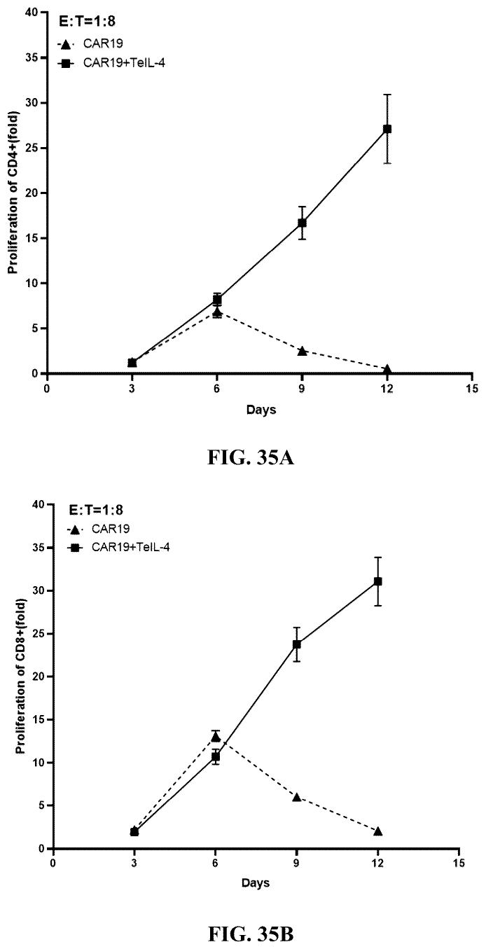

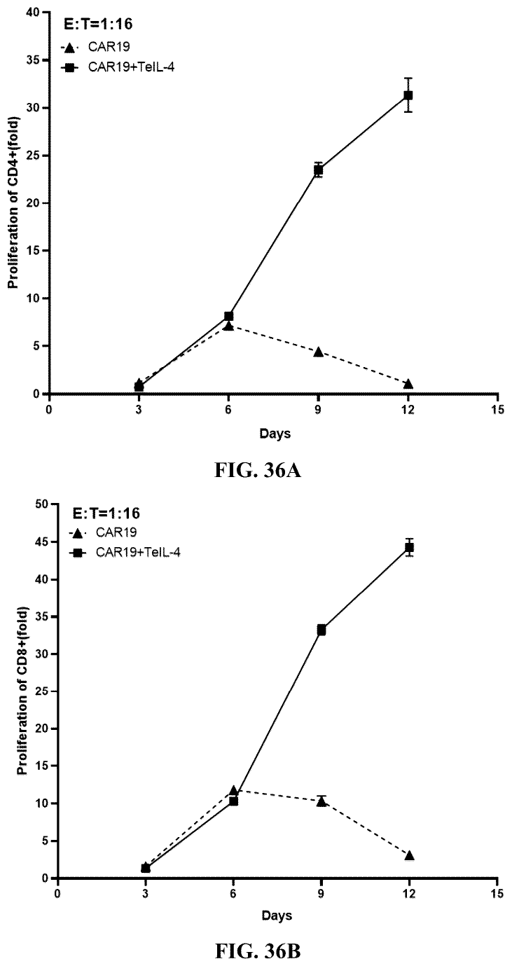

To evaluate the ability of CAR-T cells to continuously expand in vitro after killing the tumor cells. CAR19 T cells (control group. CAR19) or CAR-T cells expressing target construct (CAR19+TeIL-4) were mixed with tumor cells in 1:8 and 1:16 effector-to-tumor ratio, respectively, and was added in a 48-well plate. Conjugated APC-labeled anti-CD8 antibody, anti-CD4 antibody and FITC-labeled anti-scFv antibody were used for simultaneous staining of CD8+ CAR-T cells and CD4+ CAR-T cells, respectively. CD4+ CAR-T cells and CD8+ CAR-T cells were detected using flow cytometry (day 0), then on the third day, and every three days thereafter for 12 days. Cells were maintained at a density of 4×10⁵/mL until the T cells stopped proliferating. The cumulative proliferation fold change was calculated based on (the number of cells on day n)/(number of cells on day 0). The data of each group are shown in the line graphs as illustrated in FIGS. 35A-36B.

At E:T ratio of both 1:8 and 1:16, proliferation of both CD4+ CAR-T cells and CD8+ CAR-T cells began to decrease after day 6 in the CAR19 T cells control group. At day 12, proliferation stopped for both CD4+ CAR-T cells and CD8+ CAR-T cells in the CAR19 T cells control group. In contrast, both CD4+ CAR-T cells and CD8+ CAR-T cells showed continuous proliferation for the 12 day-duration of the assessment (FIGS. 35A-36B). As such, CAR19+TeIL-4 group demonstrated improved proliferation and viability of the engineered T cells as compared to the CAR19 T cells control group.

### Example H. Continuous T Cell Cytotoxicity Assessment

This example illustrates the process of evaluating the ability of continuous cytotoxicity of the engineered T cells (CAR19+TeIL-10)) comprising constructs described in Table A-8 and A-9. The engineered CAR-T cells were produced using the method described in Examples B and C.

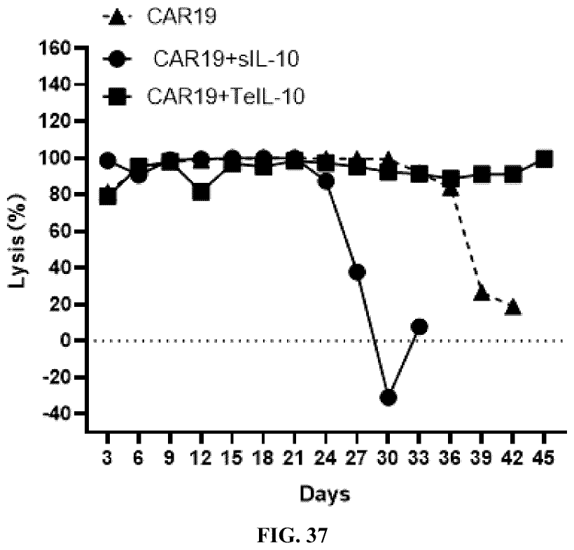

CD4+ CAR-T cells and CD8+ CAR-T cells obtained according to the method described in Example C and the cells were mixed at 1:1 ratio. CAR19 T cells (control group. CAR19). T cells expressing soluble IL-10) (control group. CAR19+sIL-10), or T cells expressing target construct (CAR19+TeIL-10) were mixed with 4.5×10⁵ Raji tumor cells expressing EGFP (Raji-GFP) in 1:8 and 1:16 effector-to-tumor ratio, respectively, and were added in a 48-well plate on day 0. Beginning on day 6, 4.5×10⁵ Raji-GFP tumor cells were added every 3 days until day 42. The plates were placed in a 37° C. 5% CO₂ incubator for co-cultivation. Flow cytometry was performed every 3 days to assess viability of T cells. Cytotoxicity was evaluated based on the number of tumor cells that were alive as compared to the total number of tumor cells after the prior round of adding tumor cells, continuous T cell cytotoxicity for each group is shown as percentage cell lysis in FIG. 37.

The ability of continuous cytotoxicity of CAR-T cells expressing target construct (CAR19+TeIL-10) was maintained at close to 100% for the 45-day duration of the assessment, while the cytotoxicity of CAR 19 T cells (control group. CAR19) and CAR-T cells expressing soluble IL-10) (control group. CAR19+sIL-10) began to decline at 36 day and 24 day, respectively (FIG. 37). As such, of T cells expressing target construct (CAR19+TeIL-10) demonstrated persistent cytotoxicity.

### Example I. Continuous T Cell Proliferation Assessment

This example illustrates the process of evaluating the ability of continuous proliferation of the engineered T cells (CAR19+TeIL-10) comprising constructs as described in Table A-8 and A-9. The engineered CAR-T cells were produced using the method described in Examples B and C.

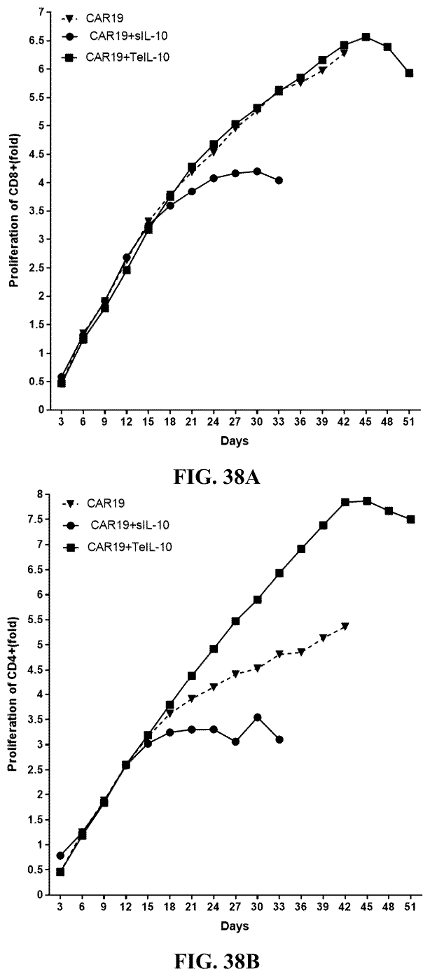

CD4+ CAR-T cells and CD8+ CAR-T cells obtained according to the method described in Example C and the cells were mixed at 1:1 ratio. CAR19-T cells (control group. CAR19). CAR-T cells expressing soluble IL-10 (control group. CAR19+sIL-10), or CAR19-T cells expressing target construct (CAR19+TeIL-10) were mixed with 4.5×10⁵ Raji tumor cells expressing EGFP (Raji-GFP) in 1:8 and 1:16 effector-to-tumor ratio, respectively, and were added in a 48-well plate on day 0. Beginning on day 6, 4.5×10⁵ Raji-GFP tumor cells were added every 3 days until day 42. The plates were placed in a 37° C. 5% CO2 incubator for co-cultivation. Flow cytometry was performed every 3 days to assess viability of CAR-T cells. The cumulative proliferation fold change was calculated based on (the number of cells on day n)/(number of cells on day 0). The data of each group are shown in the line graphs as illustrated in FIGS. 38A-B.

### Example J. In Vivo Assessment of Tumor Growth

This example illustrates the process of evaluating the effect of the engineered T cells comprising constructs described in Tables A-2. A-5. A-8, and A-9 on tumor growth in vivo. The engineered CAR-T cells were produced using the method described in Examples B and C.

Human acute lymphoblastic leukemia tumor cells NALM6-LUC, which stably express CD19 and luciferase were injected into severely immunodeficient mice (NOG mice) through tail vein injection at 2×10⁵/200 μL/animal. In vivo imaging was performed. Mice with a total fluorescent value of Total Flux≥1×10⁶ p/s were deemed as successful tumor engraftment group. 24 mice in the successful tumor engraftment group were randomly assigned into four treatment groups, and equal amount of four different test articles (vehicle. CAR19 T-cells. CAR19+TeIL-4 T cells. CAR19+TeIL-4+TeIL-15 T cells, respectively) were administered through tail vein injection. In the vehicle group (Group 1), the vehicle solution (containing 50% (v/v) CryoStor® CS10 cryopreservation media. 30% (v/v) human serum albumin (HSA). 20% (v/v) normal saline) was administered at 200 μL/animal: the CAR19-T cell group (Group 2) was administered at 3×10⁶/200 L/animal: the CAR19+TeIL-4 T cells group (Group 3) was administered at 3×10⁶/200 μL/animal: the CAR19+TeIL-4+TeIL-15 T cells group (Group 4) was administered at 3×10⁶/200 μL/animal. The CAR19+TeIL-4+TeIL-15 T cells of Group 4 was generated by transducing T cells with two viral vectors comprising constructs comprising the TeIL-4 and the TeIL-15 as described in Tables A-5 and A-2, respectively.

Efficacy was determined using average fluorescence intensity change of in vivo imaging and median survival time. Pharmacodynamic effect of the test article is determined in the tumor animal model. Detailed protocol is shown in Table J-1.

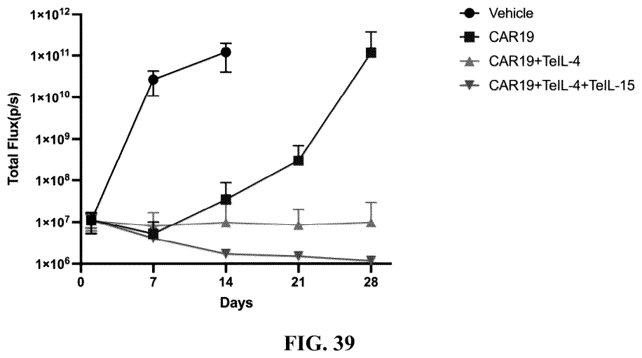

After 28 days of treatment, the average fluorescence intensity change is shown in FIG. 39. Higher Flux (p/s) value indicates higher tumor burden, which is indicative of lower tumor-killing efficacy of the test article. In the vehicle control Group 1, all mice were dead by day 14. In the CAR19-T cell group (Group 2), tumor burden increased significant after day 14, reaching the same maximum level as the tumor burden of Group 1 by day 28. In contrast, the CAR19+TeIL-4 T cells group (Group 3) and the CAR19+TeIL-4+TeIL-15 T cells group (Group 4) continued to inhibit tumor growth by day 28.

TABLE J-1
IN VIVO STUDY PROTOCOL

| Experimental | Strain | NOG | |
|---|---|---|---|
| animal | Species | Mouse | |
| | Age | 5-6 | weeks |
| | Body weight | 18-22 | g |

| | |
|---|---|
| Number/sex/group | 6/female/group |
| Total number | 24 |

| | |
|---|---|
| Test article | CAR19 + TeIL-4 T cells |
| | (T cells express CAR and Membrane anchoring IL-4) |
| | CAR19 + TeIL-4 + TeIL-15 T cells (T cells express CAR and Membrane |
| | anchoring IL-4 and Membrane anchoring IL-15; the T cells were |
| | transfected with two viral vector constructs comprising TeIL-4 and TeIL- |
| | 15, respectively) |
| Control product | Vehicle (media) |
| | Vehicle and CAR19-T cells (T cells express CAR only) |
| Route of administration | Tail vein injection |
| Descriptions of | NALM6-ZsGreen-Luc was injected to NOG mice via tail vein at 2E5/200 |
| disease/injury model and | μL/animal. Mice were then injected with the test article and controls, |
| transplantation procedures | respectively, 2 days after the tumor transplantation. |
| Study grouping and dose | Group 1: Vehicle group, 200 μL/animal |
| | Group 2: CAR19-T cells group, 3E6/200 μL/animal |
| | Group 3: CAR19 + TeIL-4 T cells group, 3E6/200 μL/animal |
| | Group 4: CAR19 + TeIL-4 + TeIL-15 T cells group, 3E6/200 μL/animal |
| Dose regimen | Single intravenous injection via tail vein |
| Randomization | Yes |
| Test | General clinical observation, twice daily. |
| | Body weight measurement, twice weekly. |
| | In vivo imaging of mice prior to administration of test articles and |
| | every week after test article infusion. |
| | Median days of survival |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the present disclosure may be employed in practicing the present disclosure. It is intended that the following claims define the scope of the present disclosure and that methods and structures within the scope of these claims and their equivalents be covered thereby.

### NUMBERED EMBODIMENTS

Embodiment 1. A nucleic acid molecule comprising a nucleic acid sequence that encodes a polypeptide comprising a signal peptide, a cytokine peptide, and a non-peptide anchor attachment signal,

wherein the cytokine peptide comprises at least a portion of IL-2, IL-4, IL-7, IL-9, IL-10, IL-18, IL-23, IL-27, IL-36γ, IL-23p19, or IL-1α, or a variant thereof.

Embodiment 2. An immune cell comprising an exogenous nucleic acid sequence encoding a polypeptide comprising a cytokine peptide and a non-peptide anchor attachment signal,

wherein the cytokine peptide comprises at least a portion of IL-2, IL-4, IL-7, IL-9, IL-10, IL-18, IL-23, IL-27, IL-36γ, IL-23p19, or IL-1α, or a variant thereof.

Embodiment 3. An immune cell comprising an exogenous nucleic acid sequence encoding a polypeptide comprising a cytokine peptide, and a non-peptide anchor attachment signal, wherein the cytokine peptide is at least a portion of IL-12p40 or at least a portion of IL-12p35 and the exogenous nucleic acid sequence does not encode both IL-12p40 and IL-12p35, and wherein the immune cell does not comprise a stimulus response element (SRE) derived from PDE5.

Embodiment 4. An immune cell comprising a protein comprising a cytokine peptide and a non-peptide anchor, wherein the cytokine peptide comprises at least a portion of IL-2, IL-4, IL-7, IL-9, IL-10, IL-18, IL-23, IL-27, IL-36γ, IL-23p19, or IL-1α, or a variant thereof.

Embodiment 5. The immune cell of embodiment 4, wherein the protein is processed from a polypeptide comprising the cytokine peptide and a non-peptide anchor attachment signal, and wherein the non-peptide anchor attachment is replaced by the non-peptide anchor during protein processing.

Embodiment 6. The immune cell of embodiment 5, wherein the polypeptide is encoded by an exogenous nucleic acid sequence.

Embodiment 7. The immune cell of any one of embodiments 2-6, wherein the polypeptide or the protein comprises a signal peptide.

Embodiment 8. The nucleic acid molecule of embodiment 1 or the immune cell of embodiment 7, wherein the signal peptide, the cytokine peptide, and the non-peptide anchor attachment signal are operably linked in a direction from a N-terminal of the polypeptide to a C-terminal of the polypeptide.

Embodiment 9. The nucleic acid molecule of embodiments 1 or 8 or the immune cell of any one of embodiments 2 or 4-8, wherein the non-peptide anchor attachment signal comprises a glycolipid-attachment signal.

Embodiment 10. The nucleic acid molecule or the immune cell of embodiment 9, wherein the glycolipid-attachment signal comprises a glycosylphosphatidylinositol (GPI)-attachment signal.

Embodiment 11. The nucleic acid molecule or the immune cell of embodiment 10, wherein the GPI-attachment signal comprises an amino acid sequence that is at least 75%, 80%, 85%, 90%, 95%, 98%, 99%, or 100% identical to the sequence set forth in SEQ ID NO: 200.

Embodiment 12. The nucleic acid molecule or the immune cell of embodiment 10, wherein the GPI-attachment signal is encoded by a nucleic acid sequence that is at least 75%, 80%, 85%, 90%, 95%, 98%, 99%, or 100% identical to any of the sequences set forth in SEQ ID NOs: 250-252.

Embodiment 13. A nucleic acid molecule comprising a nucleic acid sequence that encodes a polypeptide comprising a cytokine peptide and a peptide anchor,

wherein the cytokine peptide comprises at least a portion of IL-2, IL-4, IL-7, IL-9, IL-10, IL-18, IL-23, IL-27, IL-36γ, IL-23p19, or IL-1α, or a variant thereof.

Embodiment 14. A cell comprising the nucleic acid molecule of embodiment 13.

Embodiment 15. A cell comprising a polypeptide comprising a cytokine peptide and a peptide anchor, wherein the cytokine peptide comprises at least a portion of IL-2, IL-4, IL-7, IL-9, IL-10, IL-18, IL-23, IL-27, IL-36γ, IL-23p19, or IL-1α, or a variant thereof.

Embodiment 16. The nucleic acid molecule of embodiment 13 or the cell of embodiment 14 or 15, wherein the polypeptide comprises a signal peptide.

Embodiment 17. The nucleic acid molecule or the cell of embodiment 16, wherein the signal peptide, the cytokine peptide, and the peptide anchor are operably linked in a direction from a N-terminal of the polypeptide to a C-terminal of the polypeptide.

Embodiment 18. The nucleic acid molecule of any one of embodiments 13, 16, or 17 or the cell of any one of embodiments 14-17, wherein the peptide anchor comprises a transmembrane peptide sequence.

Embodiment 19. The nucleic acid molecule or the cell of embodiment 18, wherein the transmembrane peptide sequence comprises a B7-1 transmembrane amino acid sequence, a B7-2 transmembrane amino acid sequence. B7-H1 transmembrane amino acid sequence. B7-H3 transmembrane amino acid sequence, tumor necrosis factor receptor 2 (TNFR2) transmembrane amino acid sequence, a CD8α transmembrane amino acid sequence, a CD28 transmembrane amino acid sequence, a CD3ζ transmembrane amino acid sequence, a CTLA-4 (CD152) transmembrane amino acid sequence, or a PD-L1 transmembrane amino acid sequence, or any fragments or variants thereof.

Embodiment 20. The nucleic acid molecule or the cell of embodiment 18 or 19, wherein the transmembrane peptide sequence comprises an amino acid sequence that is at least 75%, 80%, 85%, 90%, 95%, 98%, 99%, or 100% identical to any of the sequences set forth in SEQ ID NOs: 203-204.

Embodiment 21. The nucleic acid molecule or the cell of embodiment 18 or 19, wherein the transmembrane peptide sequence is encoded by a nucleic acid sequence that is at least 75%, 80%, 85%, 90%, 95%, 98%, 99%, or 100% identical to any of the sequences set forth in SEQ ID NOs: 253-255.

Embodiment 22. The nucleic acid molecule of any one of embodiments 1, 8-12, or 16-21, the immune cell of any one of embodiments 7-12, the cell of any one of embodiments 16-21, wherein the signal peptide comprises CD4 signal peptide, CD8α signal peptide, CD28 signal peptide, CD33 signal peptide. CD137 (4-1BB) signal peptide, IL-2 signal peptide, IgE signal peptide, IgG1 signal peptide. GM-CSF signal peptide, HLA-A signal peptide, HLA signal peptide, TCR signal peptide, or β2M signal peptide, or a combination thereof.

Embodiment 23. The nucleic acid molecule, the immune cell, or the cell of embodiment 22, wherein the signal peptide comprises an amino acid sequence that is at least 75%, 80%, 85%, 90%, 95%, 98%, 99%, or 100% identical to any of the sequences set forth in SEQ ID NOs: 2-7.

Embodiment 24. The nucleic acid molecule, the immune cell, or the cell of embodiment 22, wherein the signal peptide is encoded by a nucleic acid sequence that is at least 75%, 80%, 85%, 90%, 95%, 98%, 99%, or 100% identical to any of the sequences set forth in SEQ ID NOs: 52-57.

Embodiment 25. The nucleic acid molecule of any one of embodiments 1, 8-12, or 16-21, the immune cell of any one of embodiments 7-12, the cell of any one of embodiments 16-21, wherein the signal peptide is a naturally occurring signal peptide of a wild-type cytokine.

Embodiment 26. The nucleic acid molecule, the immune cell, or the cell of embodiment 25, wherein the signal peptide comprises an IL-2 signal peptide, an IL-4 signal peptide, an IL-7 signal peptide, an IL-9 signal peptide, an IL-10 signal peptide, an IL-12p40 signal peptide, an IL-15 signal peptide, an IL-18 signal peptide, an IL-21 signal peptide, an IL-23 signal peptide, an IL-27 signal peptide, an IL-36γ signal peptide, an IL-23p19 signal peptide, or an IL-1α signal peptide, or a functional variant thereof.

Embodiment 27. The nucleic acid molecule, the immune cell, or the cell of embodiment 25, wherein the signal peptide comprises an amino acid sequence that is at least 75%, 80%, 85%, 90%, 95%, 98%, 99%, or 100% identical to the sequence set forth in SEQ ID NO: 113 or 114.

Embodiment 28. The nucleic acid molecule, the immune cell, or the cell of embodiment 25, wherein the signal peptide is encoded by a nucleic acid sequence that is at least 75%, 80%, 85%, 90%, 95%, 98%, or 99%, or 100% identical to the sequence set forth in SEQ ID NO: 58 or 59.

Embodiment 29. The nucleic acid molecule of any one of embodiments 1, 8-13, or 16-28, the immune cell of any one of embodiments 2-12 or 22-28, or the cell of any one of embodiments 14-28, wherein the cytokine peptide comprises at least a portion of IL-2 or a variant thereof.

Embodiment 30. The nucleic acid molecule of any one of embodiments 1, 8-13, or 16-28, the immune cell of any one of embodiments 2-12 or 22-28, or the cell of any one of embodiments 14-28, wherein the cytokine peptide comprises at least a portion of IL-4 or a variant thereof.

Embodiment 31. The nucleic acid molecule of any one of embodiments 1, 8-13, or 16-28, the immune cell of any one of embodiments 2-12 or 22-28, or the cell of any one of embodiments 14-28, wherein the cytokine peptide comprises at least a portion of IL-7 or a variant thereof.

Embodiment 32. The nucleic acid molecule of any one of embodiments 1, 8-13, or 16-28, the immune cell of any one of embodiments 2-12 or 22-28, or the cell of any one of embodiments 14-28, wherein the cytokine peptide comprises at least a portion of IL-9 or a variant thereof.

Embodiment 33. The nucleic acid molecule of any one of embodiments 1, 8-13, or 16-28, the immune cell of any one of embodiments 2-12 or 22-28, or the cell of any one of embodiments 14-28, wherein the cytokine peptide comprises at least a portion of IL-10 or a variant thereof.

Embodiment 34. The nucleic acid molecule of any one of embodiments 1, 8-13, or 16-28, the immune cell of any one of embodiments 2-12 or 22-28, or the cell of any one of embodiments 14-28, wherein the cytokine peptide comprises at least a portion of IL-18 or a variant thereof.

Embodiment 35. The nucleic acid molecule of any one of embodiments 1, 8-13, or 16-28, the immune cell of any one of embodiments 2-12 or 22-28, or the cell of any one of embodiments 14-28, wherein the cytokine peptide comprises at least a portion of IL-23 or a variant thereof.

Embodiment 36. The nucleic acid molecule of any one of embodiments 1, 8-13, or 16-28, the immune cell of any one of embodiments 2-12 or 22-28, or the cell of any one of embodiments 14-28, wherein the cytokine peptide comprises at least a portion of IL-27 or a variant thereof.

Embodiment 37. The nucleic acid molecule of any one of embodiments 1, 8-13, or 16-28, the immune cell of any one of embodiments 2-12 or 22-28, or the cell of any one of embodiments 14-28, wherein the cytokine peptide comprises at least a portion of IL-36γ or a variant thereof.

Embodiment 38. The nucleic acid molecule of any one of embodiments 1, 8-13, or 16-28, the immune cell of any one of embodiments 2-12 or 22-28, or the cell of any one of embodiments 14-28, wherein the cytokine peptide comprises at least a portion of IL-23p19 or a variant thereof.

Embodiment 39. The nucleic acid molecule of any one of embodiments 1, 8-13, or 16-28, the immune cell of any one of embodiments 2-12 or 22-28, or the cell of any one of embodiments 14-28, wherein the cytokine peptide comprises at least a portion of IL-1α or a variant thereof.

Embodiment 40. The nucleic acid molecule of any one of embodiments 1, 8-13, or 16-39, the immune cell of any one of embodiments 2-12 or 22-39, or the cell of any one of embodiments 14-39, wherein the variant of the cytokine peptide comprises an alteration, substitution, deletion, addition, or chemical modification of one or more amino acids, one or more unnatural amino acids, or any combination thereof.

Embodiment 41. The nucleic acid molecule of any one of embodiments 1, 8-13, or 16-40), the immune cell of any one of embodiments 2-12 or 22-40), or the cell of any one of embodiments 14-40, wherein the cytokine peptide comprises an amino acid sequence that is at least 75%, 80%, 85%, 90%, 95%, 98%, 99%, or 100% identical to any of the sequences set forth in SEQ ID NOs: 100-104, 107, or 109-112.

Embodiment 42. The nucleic acid molecule of any one of embodiments 1, 8-13, or 16-40, the immune cell of any one of embodiments 2-12 or 22-40, or the cell of any one of embodiments 14-40, wherein the cytokine peptide is encoded by a nucleic acid sequence that is at least 75%, 80%, 85%, 90%, 95%, 98%, 99%, or 100% identical to any of the sequences set forth in SEQ ID NOs: 150-154, 157, or 159-164.

Embodiment 43. The nucleic acid molecule of any one of embodiments 1, 8-13, or 16-42, the immune cell of any one of embodiments 2-12 or 22-42, or the cell of any one of embodiments 14-42, wherein the nucleic acid molecule or the immune cell or the cell further comprises a targeting sequence encoding a targeting moiety, and wherein the targeting moiety comprises a chimeric antigen receptor, a T cell receptor, a B cell receptor, or any combination thereof.

Embodiment 44. The nucleic acid molecule, the immune cell, or the cell of embodiment 43, wherein the targeting sequence is under control of a different promoter as the nucleic acid sequence or the exogenous nucleic acid sequence.

Embodiment 45. The nucleic acid molecule, the immune cell, or the cell of embodiment 43, wherein the targeting sequence is under control of a same promoter as the nucleic acid sequence or the exogenous nucleic acid sequence.

Embodiment 46. The nucleic acid molecule, the immune cell, or the cell of embodiment 43 or 44, wherein the targeting sequence encodes a chimeric antigen receptor (CAR).

Embodiment 47. The nucleic acid molecule, the immune cell, or the cell of embodiment 46, wherein the chimeric antigen receptor (CAR) comprises a ligand binding domain, and wherein the ligand binding domain targets CD19, CD20, CD22, CD30, CD33, CD38, CD70, CD123, CD138, CD171, CD5, CD7, MUC1, AFP, CEA, PSCA, PSMA, HER2, EGFR, IL13Ralpha2, GD2, NKG2D, EGFTvIII, CS1, CCL1. BCMA, Mesothelin, ROR1, FLT3, FAP, TAG72, CD38, CD44v6, CEA, EPCAM, B7H3, KIT, IL-13Ralpha2, PRSS21, VEGR2, LewisY, CD24, PDGFR-beta, SSEA-4, AFP, NCAM, Claudin18.2, GPC3, GM3, TGS5, HMWMAA, TEM7R, CLDN6, GPRC5D, CXORF61, CD97, CD179a, ALK, PLAC1, GloboH, NY-BR-1, UPK2, HAVCR1, ADRB3, PANX3, GPR20, LY6K, OR51E2, TRAP, WT1, NY-ESO-1, LAGE-1a, or MAGE-A1.

Embodiment 48. The nucleic acid molecule, the immune cell, or the cell of embodiment 46 or 47, wherein the ligand binding domain targets CD19.

Embodiment 49. The nucleic acid molecule, the immune cell, or the cell of any one of embodiments 43-48, wherein the targeting sequence encodes CAR19.

Embodiment 50. The nucleic acid molecule, the immune cell, or the cell of any one of embodiments 46-49, wherein the CAR comprises an amino acid sequence that is at least 75%, 80%, 85%, 90%, 95%, 98%, 99%, or 100% identical to any of the sequences set forth in SEQ ID NOs: 4 or 400-407.

Embodiment 51. The nucleic acid molecule, the immune cell, or the cell of any one of embodiments 46-49, wherein the CAR is encoded by a nucleic acid sequence that is at least 75%, 80%, 85%, 90%, 95%, 98%, 99%, or 100% identical to any of the sequences set forth in SEQ ID NOs: 54 or 450-457.

Embodiment 52. The nucleic acid molecule, the immune cell, or the cell of any one of embodiments 46-49, wherein the targeting sequence is linked to the nucleic acid sequence or the exogenous nucleic acid sequence via a nucleic acid sequence encoding a cleavable linker.

Embodiment 53. The nucleic acid molecule, the immune cell, or the cell of embodiment 52, wherein the cleavable linker comprises P2A peptide, T2A peptide, E2A peptide, F2A peptide, or IRES peptide.

Embodiment 54. The nucleic acid molecule, the immune cell, or the cell of embodiment 52 or 53, wherein the cleavable linker is encoded by a nucleic acid sequence having at least 75%, 80%, 85%, 90%, 95%, 98%, 99%, or 100% identical to any of the sequences set forth in SEQ ID NOs: 350-355.

Embodiment 55. The nucleic acid molecule, the immune cell, or the cell of any one of embodiments 52-54, wherein the cleavable linker connects the CAR and the signal peptide.

Embodiment 56. The nucleic acid molecule, the immune cell, or the cell of any one of embodiments 52-54, wherein the cleavable linker connects the CAR and the peptide anchor.

Embodiment 57. The nucleic acid molecule, the immune cell, or the cell of any one of embodiments 52-54, wherein the cleavable linker connects the CAR and the non-peptide anchor attachment signal.

Embodiment 58. The nucleic acid molecule, the immune cell, or the cell of any one of embodiments 52-57, wherein the cleavable linker comprises an amino acid sequence that is at least 75%, 80%, 85%, 90%, 95%, 98%, 99%, or 100% identical to any of the sequences set forth in SEQ ID NOs: 300-303.

Embodiment 59. The nucleic acid molecule of any one of embodiments 1, 8-13, or 16-58, the immune cell of any one of embodiments 2-12 or 22-58, or the cell of any one of embodiments 14-58, wherein the polypeptide or protein further comprises a peptide linker.

Embodiment 60. The nucleic acid molecule, the immune cell, or the cell of embodiment 59, wherein the peptide linker connects the cytokine peptide to the peptide anchor.

Embodiment 61. The nucleic acid molecule, the immune cell, or the cell of embodiment 59, wherein the peptide linker connects the cytokine peptide to the non-peptide anchor attachment signal.

Embodiment 62. The nucleic acid molecule, the immune cell, or the cell of embodiment 59, wherein the peptide linker connects the peptide anchor to the cleavable linker.

Embodiment 63. The nucleic acid molecule, the immune cell, or the cell of embodiment 59, wherein the peptide linker connects the non-peptide anchor attachment signal to the cleavable linker.

Embodiment 64. The nucleic acid molecule, the immune cell, or the cell of any one of embodiments 59-63, wherein the peptide linker comprises a GS linker, a Lr1 linker, or a Lr8 linker.

Embodiment 65. The nucleic acid molecule, the immune cell, or the cell of any one of embodiments 59-64, wherein the peptide linker comprises an amino acid sequence that is at least 75%, 80%, 85%, 90%, 95%, 98%, 99%, or 100% identical to any of the sequences set forth in SEQ ID NOs: 500, 501, 504, 506, or 507, or the sequence of LE, AS, GSG, or EF.

Embodiment 66. The nucleic acid molecule of any one of embodiments 1, 8-13, or 16-65, wherein the nucleic acid molecule is RNA.

Embodiment 67. The nucleic acid molecule of any one of embodiments 1, 8-13, or 16-65, wherein the nucleic acid molecule is DNA.

Embodiment 68. The nucleic acid molecule of any one of embodiments 1, 8-13, or 16-66, wherein the nucleic acid molecule is a linear RNA.

Embodiment 69. The nucleic acid molecule of any one of embodiments 1, 8-13, or 16-66, wherein the nucleic acid molecule is a circular RNA.

Embodiment 70. The nucleic acid molecule of any one of embodiments 1, 8-13, or 16-69, wherein the nucleic acid molecule is a vector.

Embodiment 71. The nucleic acid molecule of embodiment 70, wherein the vector is a viral vector.

Embodiment 72. The nucleic acid molecule of embodiment 70, wherein the vector is a retroviral vector, a lentiviral vector, an adenoviral vector, or an adeno-associated viral vector.

Embodiment 73. The cell of any one of embodiments 14-65, wherein the cell is a bacterial cell, yeast cell, or an insect cell.

Embodiment 74. The cell of any one of embodiments 14-65, wherein the cell is an immune cell or a tumor cell.

Embodiment 75. The immune cell of any one of embodiments 2-12 or 22-65 or the cell of any one of embodiments 14-65 or 74, wherein the immune cell is an engineered immune cell.

Embodiment 76. The immune cell of any one of embodiments 2-12 or 22-65 or the cell of any one of embodiments 14-65 or 74, wherein the immune cell is a T cell.

Embodiment 77. The immune cell of any one of embodiments 2-12 or 22-65 or the cell of any one of embodiments 14-65 or 74, wherein the immune cell is a tumor infiltrating lymphocyte (TIL).

Embodiment 78. The immune cell or the cell of embodiment 75, wherein the engineered immune cell is a natural killer (NK) cell.

Embodiment 79. The immune cell of any one of embodiments 2-12, 22-65, or 75-78 or the cell of any one of embodiments 14-65 or 74-78, wherein the targeting sequence and the nucleic acid sequence are present in a same plasmid inside the immune cell.

Embodiment 80. The immune cell of any one of embodiments 2-12, 22-65, or 75-78 or the cell of any one of embodiments 14-65 or 74-78, wherein the targeting sequence and the nucleic acid sequence are present in two different plasmids inside the immune cell.

Embodiment 81. The immune cell of any one of embodiments 2-12, 22-65, or 75-80 or the cell of any one of embodiments 14-65 or 74-80, wherein the targeting sequence and the nucleic acid sequence are present in a genome of the immune cell.

Embodiment 82. The immune cell of any one of embodiments 2-12, 22-65, or 75-81 or the cell of any one of embodiments 14-65 or 74-81, wherein cytotoxicity of said immune cell is increased relative to a comparable immune cell that
(a) does not comprise the exogenous nucleic acid sequence encoding the polypeptide comprising the cytokine peptide and the non-peptide anchor attachment signal;
(b) does not comprise the nucleic acid sequence encoding the polypeptide comprising the signal peptide, the cytokine peptide and the non-peptide anchor attachment signal;
(c) does not comprise the nucleic acid sequence encoding the polypeptide comprising the cytokine peptide and the peptide anchor; or
(d) does not comprise protein comprising the cytokine peptide and (i) the non-peptide anchor or (ii) the peptide anchor.

Embodiment 83. The immune cell or the cell of embodiment 82, wherein said increase in cytotoxicity of said immune cell is measured by an in vitro cytotoxicity assay described in Examples A-D.

Embodiment 84. The immune cell or the cell of embodiment 82, wherein said increase in cytotoxicity of said immune cell is measured in vitro or in vivo.

Embodiment 85. The immune cell or the cell of embodiment 82, wherein said increase in cytotoxicity of said immune cell is at least about 5%, 10%, 20%, 30%, 40%, 50% or more.

Embodiment 86. The immune cell of any one of embodiments 2-12, 22-65, or 75-85 or the cell of any one of embodiments 14-65 or 74-85, wherein a population of said immune cells proliferates for a longer period of time relative to a population of comparable immune cells that
(a) does not comprise the exogenous nucleic acid sequence encoding the polypeptide comprising the cytokine peptide and the non-peptide anchor attachment signal;
(b) does not comprise the nucleic acid sequence encoding the polypeptide comprising the signal peptide, the cytokine peptide and the non-peptide anchor attachment signal;
(c) does not comprise the nucleic acid sequence encoding the polypeptide comprising the cytokine peptide and the peptide anchor; or
(d) does not comprise protein comprising the cytokine peptide and (i) the non-peptide anchor or (ii) the peptide anchor.

Embodiment 87. The immune cell or the cell of embodiment 86, wherein proliferation of said population of said immune cells is measured by an in vitro immune cell proliferation assay described in Examples A-E.

Embodiment 88. The immune cell or the cell of embodiment 86, wherein proliferation of said population of said immune cells is measured in vitro or in vivo.

Embodiment 89. The immune cell or the cell of embodiment 86, wherein proliferation of said population of said immune cells lasts for a period of time at least about 5%, 10%, 20%, 30%, 40%, 50%, 100%, 2 times, 3 times, 5 times, 10 times or longer than said population of said comparable immune cells.

Embodiment 90. A nucleic acid molecule comprising a first nucleic acid sequence and a second nucleic acid sequence, wherein:
(a) the first nucleic acid sequence encodes a first polypeptide comprising a first cytokine peptide and (i) a first non-peptide anchor attachment signal or (ii) a first peptide anchor;
(b) the second nucleic acid sequence encodes a second polypeptide comprising a second cytokine peptide and (i) a second non-peptide anchor attachment signal or (ii) a second peptide anchor; and wherein each of the first cytokine peptide and the second cytokine peptide independently comprises at least a portion of IL-2, IL-4, IL-7, IL-9, IL-10, IL-12, IL-12p40, IL-15, IL-18, IL-23, IL-27, IL-36γ, IL-23p19, or IL-1α or a variant thereof.

Embodiment 91. A nucleic acid molecule comprising a first nucleic acid sequence and a second nucleic acid sequence, wherein:
(a) the first nucleic acid sequence encodes a first polypeptide comprising a first cytokine peptide and (i) a first non-peptide anchor attachment signal or (ii) a first peptide anchor;
(b) the second nucleic acid sequence encodes a second polypeptide comprising a second cytokine peptide and (i) a second non-peptide anchor attachment signal or (ii) a second peptide anchor; and wherein each of the first cytokine peptide and the second cytokine peptide independently comprises at least a portion of IL-2, IL-4, IL-7, IL-9, IL-10, IL-12, IL-12p40, IL-18, IL-21, IL-23, IL-27, IL-36γ, IL-23p19, or IL-1α or a variant thereof.

Embodiment 92. A system comprising a first nucleic acid sequence and a different second nucleic acid sequence, wherein:
(a) the first nucleic acid sequence encodes a first polypeptide comprising a first cytokine peptide and (i) a first non-peptide anchor attachment signal or (ii) a first peptide anchor;
(b) the second nucleic acid sequence encodes a second polypeptide comprising a second cytokine peptide and (i) a second non-peptide anchor attachment signal or (ii) a second peptide anchor; and wherein each of the first cytokine peptide and the second cytokine peptide independently comprises at least a portion of IL-2, IL-4, IL-7, IL-9, IL-10, IL-12, IL-12p40, IL-15, IL-18, IL-23, IL-27, IL-36γ, IL-23p19, or IL-1α or a variant thereof.

Embodiment 93. A system comprising a first nucleic acid sequence and a different second nucleic acid sequence, wherein:
(a) the first nucleic acid sequence encodes a first polypeptide comprising a first cytokine peptide and (i) a first non-peptide anchor attachment signal or (ii) a first peptide anchor;
(b) the second nucleic acid sequence encodes a second polypeptide comprising a second cytokine peptide and (i) a second non-peptide anchor attachment signal or (ii) a second peptide anchor; and wherein each of the first cytokine peptide and the second cytokine peptide independently comprises at least a portion of IL-2, IL-4, IL-7, IL-9, IL-10, IL-12, IL-12p40, IL-18, IL-21, IL-23, IL-27, IL-36γ, IL-23p19, or IL-1α or a variant thereof.

Embodiment 94. A nucleic acid molecule comprising a first nucleic acid sequence and a second nucleic acid sequence, wherein:
(a) the first nucleic acid sequence encodes a first polypeptide comprising a first cytokine peptide and (i) a first non-peptide anchor attachment signal or (ii) a first peptide anchor;
(b) the second nucleic acid sequence encodes a second polypeptide comprising a second cytokine peptide and (i) a second non-peptide anchor attachment signal or (ii) a second peptide anchor; and wherein the first cytokine peptide is a proinflammatory cytokine peptide, and the second cytokine peptide is an anti-inflammatory cytokine peptide.

Embodiment 95. A system comprising a first nucleic acid sequence and a different second nucleic acid sequence, wherein:
(a) the first nucleic acid sequence encodes a first polypeptide comprising a first cytokine peptide and (i) a first non-peptide anchor attachment signal or (ii) a first peptide anchor;
(b) the second nucleic acid sequence encodes a second polypeptide comprising a second cytokine peptide and (i) a second non-peptide anchor attachment signal or (ii) a second peptide anchor; and wherein the first cytokine peptide is a proinflammatory cytokine peptide, and the second cytokine peptide is an anti-inflammatory cytokine peptide.

Embodiment 96. A cell comprising the nucleic acid molecule of embodiment 90, 91, or 94.

Embodiment 97. A cell comprising a first protein and a second protein.
wherein the first protein comprises a first cytokine peptide and (i) a first non-peptide anchor or (ii) a first peptide anchor;
wherein the second protein comprises a second cytokine peptide and (i) a second non-peptide anchor or (ii) a second peptide anchor; and
wherein each of the first cytokine peptide and the second cytokine peptide independently comprises at least a portion of IL-2, IL-4, IL-7, IL-9, IL-10, IL-12, IL-12p40, IL-15, IL-18, IL-23, IL-27, IL-36γ, IL-23p19, or IL-1α or a variant thereof.

Embodiment 98. A cell comprising a first protein and a second protein.
wherein the first protein comprises a first cytokine peptide and (i) a first non-peptide anchor or (ii) a first peptide anchor;
wherein the second protein comprises a second cytokine peptide and (i) a second non-peptide anchor or (ii) a second peptide anchor; and
wherein each of the first cytokine peptide and the second cytokine peptide independently comprises at least a portion of IL-2, IL-4, IL-7, IL-9, IL-10, IL-12, IL-12p40, IL-18, IL-21, IL-23, IL-27, IL-36γ, IL-23p19, or IL-1α or a variant thereof.

Embodiment 99. A cell comprising a first protein and a second protein,
wherein the first protein comprises a first cytokine peptide and (i) a first non-peptide anchor or (ii) a first peptide anchor;
wherein the second protein comprises a second cytokine peptide and (i) a second non-peptide anchor or (ii) a second peptide anchor; and
wherein the first cytokine peptide is a proinflammatory cytokine peptide, and the second cytokine peptide is an anti-inflammatory cytokine peptide.

Embodiment 100. The cell of any one of embodiments 97-99, wherein the first protein is processed from a first polypeptide comprising the first cytokine peptide and (i) a first non-peptide anchor attachment signal or (ii) the first peptide anchor, wherein the first non-peptide anchor attachment signal is replaced by the first non-peptide anchor during protein processing.

Embodiment 101. The cell of embodiment 100, wherein the first polypeptide is encoded by a first nucleic acid sequence.

Embodiment 102. The cell of any one of embodiments 97-101, wherein the second protein is processed from a second polypeptide comprising the second cytokine peptide and (i) a second non-peptide anchor attachment signal or (ii) the second peptide anchor, wherein the second non-peptide anchor attachment signal is replaced by the second non-peptide anchor during protein processing.

Embodiment 103. The cell of embodiment 102, wherein the second polypeptide is encoded by a second nucleic acid sequence.

Embodiment 104. The nucleic acid molecule of embodiment 90, 91, or 94, the system of embodiment 92, 93, or 95, or the cell of embodiment 96 or 103, wherein the second nucleic acid sequence and the first nucleic acid sequence are under control of a same promoter.

Embodiment 105. The nucleic acid molecule of embodiment 90, 91, or 94, the system of embodiment 92, 93, or 95, or the cell of embodiment 96 or 103, wherein the second nucleic acid sequence and the first nucleic acid sequence are under control of two different promoters.

Embodiment 106. The nucleic acid molecule of any one of embodiments 90, 91, 94, or 104-105, the system of any one of embodiments 92, 93, 95, or 104-105, or the cell of any one of embodiments 96 or 103-105, wherein the first nucleic acid sequence and the second nucleic acid sequence are operably linked in a 5′ to 3′ direction.

Embodiment 107. The nucleic acid molecule of any one of embodiments 90, 91, 94, or 104-105, the system of any one of embodiments 92, 93, 95, or 104-105, or the cell of any one of embodiments 96 or 103-105, wherein the second nucleic acid sequence and the first nucleic acid sequence are operably linked in a 5′ to 3′ direction.

Embodiment 108. The nucleic acid molecule of any one of embodiments 90, 91, 94, or 104-107, the system of any one of embodiments 92, 93, 95, or 104-107, or the cell of any one of embodiments 96 or 103-107, wherein the second nucleic acid sequence and the first nucleic acid sequence are linked via a nucleic acid sequence encoding a cleavable linker.

Embodiment 109. The nucleic acid molecule of any one of embodiments 90, 91, 94, or 104-108, the system of any one of embodiments 92, 93, 95, or 104-108, or the cell of any one of embodiments 96-108, wherein the first protein or the first polypeptide comprises a first signal peptide.

Embodiment 110. The nucleic acid molecule, the system, or the cell of embodiment 109, wherein the first signal peptide, the first cytokine peptide, and (i) the first non-peptide anchor attachment signal or (ii) the first peptide anchor are operably linked in a direction from a N-terminal of the polypeptide to a C-terminal of the polypeptide.

Embodiment 111. The nucleic acid molecule of any one of embodiments 90, 91, 94, or 104-110, the system of any one of embodiments 92, 93, 95, or 104-110, or the cell of any one of embodiments 96-110, wherein the second protein or the second polypeptide comprises a second signal peptide.

Embodiment 112. The nucleic acid molecule, the system, or the cell of embodiment 111, wherein the second signal peptide, the second cytokine peptide, and (i) the second non-peptide anchor attachment signal or (ii) the second peptide anchor are operably linked in a direction from a N-terminal of the polypeptide to a C-terminal of the polypeptide.

Embodiment 113. The nucleic acid molecule, the system, or the cell of any one of embodiments 109-112, wherein each of the first signal peptide and the second signal peptide independently comprises CD4 signal peptide, CD8α signal peptide, CD28 signal peptide, CD33 signal peptide, CD137 (4-1BB) signal peptide, IL-2 signal peptide, IgE signal peptide, IgG1 signal peptide. GM-CSF signal peptide, HLA-A signal peptide, HLA signal peptide, TCR signal peptide, or β2M signal peptide, or a combination thereof.

Embodiment 114. The nucleic acid molecule, the system, or the cell of any one of embodiments 109-113, wherein each of the first signal peptide and the second signal peptide independently comprises an amino acid sequence that is at least 75%, 80%, 85%, 90%, 95%, 98%, 99%, or 100% identical to any of the sequences set forth in SEQ ID NOs: 2-7.

Embodiment 115. The nucleic acid molecule, the system, or the cell of any one of embodiments 109-113, wherein each of the first signal peptide and the second signal peptide is independently encoded by a nucleic acid sequence that is at least 75%, 80%, 85%, 90%, 95%, 98%, 99%, or 100% identical to any of the sequences set forth in SEQ ID NOs: 52-57.

Embodiment 116. The nucleic acid molecule of any one of embodiments 90, 91, 94, or 104-115, the system of any one of embodiments 92, 93, 95, or 104-115, or the cell of any one of embodiments 96 or 100-115, wherein the first non-peptide anchor attachment signal comprises a glycolipid-attachment signal.

Embodiment 117. The nucleic acid molecule of any one of embodiments 90, 91, 94, or 104-116, the system of any one of embodiments 92, 93, 95, or 104-116, or the cell of any one of embodiments 96 or 100-116, wherein the second non-peptide anchor attachment signal comprises a glycolipid-attachment signal.

Embodiment 118. The nucleic acid molecule, the system, or the cell of embodiment 116 or 117, wherein the glycolipid-attachment signal comprises a GPI-attachment signal.

Embodiment 119. The nucleic acid molecule, the system, or the cell of embodiment 118, wherein the GPI-attachment signal comprises an amino acid sequence that is at least 75%, 80%, 85%, 90%, 95%, 98%, 99%, or 100% identical to the sequence set forth in SEQ ID NO: 200.

Embodiment 120. The nucleic acid molecule, the system, or the cell of embodiment 118, wherein the GPI-attachment signal is encoded by a nucleic acid sequence that is at least 75%, 80%, 85%, 90%, 95%, 98%, 99%, or 100% identical to any of the sequences set forth in SEQ ID NOs: 250-252.

Embodiment 121. The nucleic acid molecule of any one of embodiments 90, 91, 94, or 104-120, the system of any one of embodiments 92, 93, or 95, or 104-120, or the cell of any one of embodiments 96-120, wherein the first peptide anchor and/or the second peptide anchor comprises a transmembrane peptide sequence.

Embodiment 122. The nucleic acid molecule, the system, or the cell of embodiment 121, wherein the transmembrane peptide sequence comprises a B7-1 transmembrane amino acid sequence, a B7-2 transmembrane amino acid sequence. B7-H1 transmembrane amino acid sequence. B7-H3 transmembrane amino acid sequence, tumor necrosis factor receptor 2 (TNFR2) transmembrane amino acid sequence, a CD8α transmembrane amino acid sequence, a CD28 transmembrane amino acid sequence, a CD3ζ transmembrane amino acid sequence, a CTLA-4 (CD152) transmembrane amino acid sequence, or a PD-L1 transmembrane amino acid sequence, or any fragments or variants thereof.

Embodiment 123. The nucleic acid molecule, the system, or the cell of embodiment 121 or 122, wherein the transmembrane peptide sequence comprises an amino acid sequence that is at least 75%, 80%, 85%, 90%, 95%, 98%, 99%, or 100% identical to any of the sequences set forth in SEQ ID NOs: 203-204.

Embodiment 124. The nucleic acid molecule, the system, or the cell of embodiment 121 or 122, wherein the transmembrane peptide sequence is encoded by a nucleic acid sequence that is at least 75%, 80%, 85%, 90%, 95%, 98%, 99%, or 100% identical to any of the sequences set forth in SEQ ID NOs: 253-255.

Embodiment 125. The nucleic acid molecule of any one of embodiments 94 or 104-124, the system of any one of embodiments 95 or 104-124, or the cell of any one of embodiments 99-124, wherein the anti-inflammatory cytokine peptide comprises at least a portion of IL-4, IL-10, or IL-27, or a variant thereof, and wherein the proinflammatory cytokine peptide comprises at least a portion of IL-2, IL-7, IL-9, IL-12, IL-12p40, IL-15, IL-18, IL-21, IL-23, IL-36γ, IL-23p19, or IL-1α, or a variant thereof.

Embodiment 126. The nucleic acid molecule of any one of embodiments 94 or 104-124, the system of any one of embodiments 95 or 104-124, or the cell of any one of embodiments 99-124, wherein the anti-inflammatory cytokine peptide comprises at least a portion of IL-4 or a variant thereof, and wherein the pro-inflammatory cytokine peptide comprises at least a portion of IL-2, IL-7, IL-9, IL-12, IL-12p40, IL-15, IL-18, IL-21, IL-23, IL-27, IL-36γ, IL-23p19, or IL-1α or a variant thereof.

Embodiment 127. The nucleic acid molecule of any one of embodiments 94 or 104-124, the system of any one of embodiments 95 or 104-124, or the cell of any one of embodiments 99-124, wherein the anti-inflammatory cytokine peptide comprises an amino acid sequence that is at least 75%, 80%, 85%, 90%, 95%, 98%, 99%, or 100% identical to the sequence set forth in SEQ ID NO: 101, 104, or 109, and wherein the proinflammatory cytokine peptide comprises an amino acid sequence that is at least 75%, 80%, 85%, 90%, 95%, 98%, 99%, or 100% identical to any one of the sequences set forth in SEQ ID NOs: 100, 102, 103, 105-108, or 110-112.

Embodiment 128. The nucleic acid molecule of any one of embodiments 94 or 104-124, the system of any one of embodiments 95 or 104-124, or the cell of any one of embodiments 99-124, wherein the anti-inflammatory cytokine peptide is encoded by a nucleic acid sequence that is at least 75%, 80%, 85%, 90%, 95%, 98%, 99%, or 100% identical to the sequence set forth in SEQ ID NO: 151, 163, 154, 164, or 159, and wherein the proinflammatory cytokine peptide is encoded by a nucleic acid sequence that is at least 75%, 80%, 85%, 90%, 95%, 98%, 99%, or 100% identical to any one of the sequences set forth in SEQ ID NOs: 150, 152, 153, 155-158, or 160-162.

Embodiment 129. The nucleic acid molecule of any one of embodiments 94 or 104-124, the system of any one of embodiments 95 or 104-124, or the cell of any one of embodiments 99-124, wherein the anti-inflammatory cytokine peptide comprises an amino acid sequence that is at least 75%, 80%, 85%, 90%, 95%, 98%, 99%, or 100% identical to the sequence set forth in SEQ ID NO: 101, and wherein the pro-inflammatory cytokine peptide comprises an amino acid sequence that is at least 75%, 80%, 85%, 90%, 95%, 98%, 99%, or 100% identical to any one of the sequences set forth in SEQ ID NOs: 100, 102, 103, 105-108, or 110-112.

Embodiment 130. The nucleic acid molecule of any one of embodiments 94 or 104-124, the system of any one of embodiments 95 or 104-124, or the cell of any one of embodiments 99-124, wherein the anti-inflammatory cytokine peptide is encoded by a nucleic acid sequence that is at least 75%, 80%, 85%, 90%, 95%, 98%, 99%, or 100% identical to the sequence set forth in SEQ ID NO: 151 or 163, and wherein the proinflammatory cytokine peptide is encoded by a nucleic acid sequence that is at least 75%, 80%, 85%, 90%, 95%, 98%, 99%, or 100% identical to any one of the sequences set forth in SEQ ID NOs: 150, 152, 153, 155-158, or 160-162.

Embodiment 131. The nucleic acid molecule of any one of embodiments 90 or 104-124, the system of any one of embodiments 92 or 104-124, or the cell of any one of embodiments 96-97 or 99-124, wherein the first cytokine peptide comprises at least a portion of IL-4 or a variant thereof, and wherein the second cytokine peptide comprises at least a portion of IL-2, IL-7, IL-9, IL-10, IL-12, IL-12p40, IL-15, IL-18, IL-23, IL-27, IL-36γ, IL-23p19, or IL-1α or a variant thereof.

Embodiment 132. The nucleic acid molecule of any one of embodiments 90 or 104-124, the system of any one of embodiments 92 or 104-124, or the cell of any one of embodiments 96-97 or 99-124, wherein the first cytokine peptide comprises an amino acid sequence that is at least 75%, 80%, 85%, 90%, 95%, 98%, 99%, or 100% identical to the sequence set forth in SEQ ID NO: 101, and wherein the second cytokine peptide comprises an amino acid sequence that is at least 75%, 80%, 85%, 90%, 95%, 98%, 99%, or 100% identical to any one of the sequences set forth in SEQ ID NOs: 100, 102-107, or 109-112.

Embodiment 133. The nucleic acid molecule of any one of embodiments 90 or 104-124, the system of any one of embodiments 92 or 104-124, or the cell of any one of embodiments 96-97 or 99-124, wherein the first cytokine peptide is encoded by a nucleic acid sequence that is at least 75%, 80%, 85%, 90%, 95%, 98%, 99%, or 100% identical to the sequence set forth in SEQ ID NO: 151 or 163, and wherein the second cytokine peptide is encoded by a nucleic acid sequence that is at least 75%, 80%, 85%, 90%, 95%, 98%, 99%, or 100% identical to any one of the sequences set forth in SEQ ID NOs: 150, 152-157, 159-162, or 164.

Embodiment 134. The nucleic acid molecule of any one of embodiments 91 or 104-124, the system of any one of embodiments 93 or 104-124, or the cell of any one of embodiments 96 or 98-124, wherein the first cytokine peptide comprises at least a portion of IL-4 or a variant thereof, and wherein the second cytokine peptide comprises at least a portion of IL-2, IL-7, IL-9, IL-10, IL-12, IL-12p40, IL-18, IL-21, IL-23, IL-27, IL-36γ, IL-23p19, or IL-1α or a variant thereof.

Embodiment 135. The nucleic acid molecule of any one of embodiments 91 or 104-124, the system of any one of embodiments 93 or 104-124, or the cell of any one of embodiments 96 or 98-124, wherein the first cytokine peptide comprises an amino acid sequence that is at least 75%, 80%, 85%, 90%, 95%, 98%, 99%, or 100% identical to the sequence set forth in SEQ ID NO: 101, and wherein the second cytokine peptide comprises an amino acid sequence that is at least 75%, 80%, 85%, 90%, 95%, 98%, 99%, or 100% identical to any one of the sequences set forth in SEQ ID NOs: 100, 102-105, or 107-112.

Embodiment 136. The nucleic acid molecule of any one of embodiments 91 or 104-124, the system of any one of embodiments 93 or 104-124, or the cell of any one of embodiments 96 or 98-124, wherein the first cytokine peptide is encoded by a nucleic acid sequence that is at least 75%, 80%, 85%, 90%, 95%, 98%, 99%, or 100% identical to the sequence set forth in SEQ ID NO: 151 or 163, and wherein the second cytokine peptide is encoded by a nucleic acid sequence that is at least 75%, 80%, 85%, 90%, 95%, 98%, 99%, or 100% identical to any one of the sequences set forth in SEQ ID NOs: 150, 152-155, 157-162, or 164.

Embodiment 137. The nucleic acid molecule of any one of embodiments 90-91 or 104-124, the system of any one of embodiments 92-93 or 104-124, or the cell of any one of embodiments 96-124, wherein the first cytokine peptide comprises at least a portion of IL-2 or a variant thereof.

Embodiment 138. The nucleic acid molecule of any one of embodiments 90-91 or 104-124, the system of any one of embodiments 92-93 or 104-124, or the cell of any one of embodiments 96-124, wherein the first cytokine peptide comprises at least a portion of IL-4 or a variant thereof.

Embodiment 139. The nucleic acid molecule of any one of embodiments 90-91 or 104-124, the system of any one of embodiments 92-93 or 104-124, or the cell of any one of embodiments 96-124, wherein the first cytokine peptide comprises at least a portion of IL-7 or a variant thereof.

Embodiment 140. The nucleic acid molecule of any one of embodiments 90-91 or 104-124, the system of any one of embodiments 92-93 or 104-124, or the cell of any one of embodiments 96-124, wherein the first cytokine peptide comprises at least a portion of IL-9 or a variant thereof.

Embodiment 141. The nucleic acid molecule of any one of embodiments 90-91 or 104-124, the system of any one of embodiments 92-93 or 104-124, or the cell of any one of embodiments 96-124, wherein the first cytokine peptide comprises at least a portion of IL-10 or a variant thereof.

Embodiment 142. The nucleic acid molecule of any one of embodiments 90-91 or 104-124, the system of any one of embodiments 92-93 or 104-124, or the cell of any one of embodiments 96-124, wherein the first cytokine peptide comprises at least a portion of IL-12 or a variant thereof.

Embodiment 143. The nucleic acid molecule of any one of embodiments 90-91 or 104-124, the system of any one of embodiments 92-93 or 104-124, or the cell of any one of embodiments 96-124, wherein the first cytokine peptide comprises at least a portion of IL-12p40 or a variant thereof.

Embodiment 144. The nucleic acid molecule of any one of embodiments 90-91 or 104-124, the system of any one of embodiments 92-93 or 104-124, or the cell of any one of embodiments 96-124, wherein the first cytokine peptide comprises at least a portion of IL-18 or a variant thereof.

Embodiment 145. The nucleic acid molecule of any one of embodiments 90-91 or 104-124, the system of any one of embodiments 92-93 or 104-124, or the cell of any one of embodiments 96-124, wherein the first cytokine peptide comprises at least a portion of IL-23 or a variant thereof.

Embodiment 146. The nucleic acid molecule of any one of embodiments 90-91 or 104-124, the system of any one of embodiments 92-93 or 104-124, or the cell of any one of embodiments 96-124, wherein the first cytokine peptide comprises at least a portion of IL-27 or a variant thereof.

Embodiment 147. The nucleic acid molecule of any one of embodiments 90-91 or 104-124, the system of any one of embodiments 92-93 or 104-124, or the cell of any one of embodiments 96-124, wherein the first cytokine peptide comprises at least a portion of IL-36γ or a variant thereof.

Embodiment 148. The nucleic acid molecule of any one of embodiments 90-91 or 104-124, the system of any one of embodiments 92-93 or 104-124, or the cell of any one of embodiments 96-124, wherein the first cytokine peptide comprises at least a portion of IL-23p19 or a variant thereof.

Embodiment 149. The nucleic acid molecule of any one of embodiments 90-91 or 104-124, the system of any one of embodiments 92-93 or 104-124, or the cell of any one of embodiments 96-124, wherein the first cytokine peptide comprises at least a portion of IL-1α or a variant thereof.

Embodiment 150. The nucleic acid molecule of any one of embodiments 90 or 104-124, the system of any one of embodiments 92 or 104-124, or the cell of any one of embodiments 96-97 or 100-124, wherein the first cytokine peptide comprises at least a portion of IL-15 or a variant thereof.

Embodiment 151. The nucleic acid molecule of any one of embodiments 91 or 104-124, the system of any one of embodiments 93 or 104-124, or the cell of any one of embodiments 96 or 98-124, wherein the first cytokine peptide comprises at least a portion of IL-21 or a variant thereof.

Embodiment 152. The nucleic acid molecule of any one of embodiments 90 or 104-124, the system of any one of embodiments 92 or 104-124, or the cell of any one of embodiments 96-97 or 100-124, wherein the first cytokine peptide comprises an amino acid sequence that is at least 75%, 80%, 85%, 90%, 95%, 98%, 99%, or 100% identical to any of the sequences set forth in SEQ ID NOs: 100-107 or 109-112.

Embodiment 153. The nucleic acid molecule of any one of embodiments 90 or 104-124, the system of any one of embodiments 92 or 104-124, or the cell of any one of embodiments 96-97 or 100-124, wherein the first cytokine peptide is encoded by a nucleic acid sequence that is at least 75%, 80%, 85%, 90%, 95%, 98%, 99%, or 100% identical to any of the sequences set forth in SEQ ID NOs: 150-157 or 159-164.

Embodiment 154. The nucleic acid molecule of any one of embodiments 91 or 104-124, the system of any one of embodiments 93 or 104-124, or the cell of any one of embodiments 96 or 98-124, wherein the first cytokine peptide comprises an amino acid sequence that is at least 75%, 80%, 85%, 90%, 95%, 98%, 99%, or 100% identical to any of the sequences set forth in SEQ ID NOs: 100-105 or 107-112.

Embodiment 155. The nucleic acid molecule of any one of embodiments 91 or 104-124, the system of any one of embodiments 93 or 104-124, or the cell of any one of embodiments 96 or 98-124, wherein the first cytokine peptide is encoded by a nucleic acid sequence that is at least 75%, 80%, 85%, 90%, 95%, 98%, 99%, or 100% identical to any of the sequences set forth in SEQ ID NOs: 150-155 or 157-164.

Embodiment 156. The nucleic acid molecule of any one of embodiments 90-91 or 104-155, the system of any one of embodiments 92-93 or 104-155, or the cell of any one of embodiments 96-155, wherein the second cytokine peptide comprises at least a portion of IL-2 or a variant thereof.

Embodiment 157. The nucleic acid molecule of any one of embodiments 90-91 or 104-155, the system of any one of embodiments 92-93 or 104-155, or the cell of any one of embodiments 96-155, wherein the second cytokine peptide comprises at least a portion of IL-4 or a variant thereof.

Embodiment 158. The nucleic acid molecule of any one of embodiments 90-91 or 104-155, the system of any one of embodiments 92-93 or 104-155, or the cell of any one of embodiments 96-155, wherein the second cytokine peptide comprises at least a portion of IL-7 or a variant thereof.

Embodiment 159. The nucleic acid molecule of any one of embodiments 90-91 or 104-155, the system of any one of embodiments 92-93 or 104-155, or the cell of any one of embodiments 96-155, wherein the second cytokine peptide comprises at least a portion of IL-9 or a variant thereof.

Embodiment 160. The nucleic acid molecule of any one of embodiments 90-91 or 104-155, the system of any one of embodiments 92-93 or 104-155, or the cell of any one of embodiments 96-155, wherein the second cytokine peptide comprises at least a portion of IL-10 or a variant thereof.

Embodiment 161. The nucleic acid molecule of any one of embodiments 90-91 or 104-155, the system of any one of embodiments 92-93 or 104-155, or the cell of any one of embodiments 96-155, wherein the second cytokine peptide comprises at least a portion of IL-12 or a variant thereof.

Embodiment 162. The nucleic acid molecule of any one of embodiments 90-91 or 104-155, the system of any one of embodiments 92-93 or 104-155, or the cell of any one of embodiments 96-155, wherein the second cytokine peptide comprises at least a portion of IL-12p40 or a variant thereof.

Embodiment 163. The nucleic acid molecule of any one of embodiments 90-91 or 104-155, the system of any one of embodiments 92-93 or 104-155, or the cell of any one of embodiments 96-155, wherein the second cytokine peptide comprises at least a portion of IL-18 or a variant thereof.

Embodiment 164. The nucleic acid molecule of any one of embodiments 90-91 or 104-155, the system of any one of embodiments 92-93 or 104-155, or the cell of any one of embodiments 96-155, wherein the second cytokine peptide comprises at least a portion of IL-23 or a variant thereof.

Embodiment 165. The nucleic acid molecule of any one of embodiments 90-91 or 104-155, the system of any one of embodiments 92-93 or 104-155, or the cell of any one of embodiments 96-155, wherein the second cytokine peptide comprises at least a portion of IL-27 or a variant thereof.

Embodiment 166. The nucleic acid molecule of any one of embodiments 90-91 or 104-155, the system of any one of embodiments 92-93 or 104-155, or the cell of any one of embodiments 96-155, wherein the second cytokine peptide comprises at least a portion of IL-36γ or a variant thereof.

Embodiment 167. The nucleic acid molecule of any one of embodiments 90-91 or 104-155, the system of any one of embodiments 92-93 or 104-155, or the cell of any one of embodiments 96-155, wherein the second cytokine peptide comprises at least a portion of IL-23p19 or a variant thereof.

Embodiment 168. The nucleic acid molecule of any one of embodiments 90-91 or 104-155, the system of any one of embodiments 92-93 or 104-155, or the cell of any one of embodiments 96-155, wherein the second cytokine peptide comprises at least a portion of IL-1α or a variant thereof.

Embodiment 169. The nucleic acid molecule of any one of embodiments 90 or 104-155, the system of any one of embodiments 92 or 104-155, or the cell of any one of embodiments 96-97 or 100-155, wherein the second cytokine peptide comprises at least a portion of IL-15 or a variant thereof.

Embodiment 170. The nucleic acid molecule of any one of embodiments 91 or 104-155, the system of any one of embodiments 93 or 104-155, or the cell of any one of embodiments 96 or 98-155, wherein the second cytokine peptide comprises at least a portion of IL-21 or a variant thereof.

Embodiment 171. The nucleic acid molecule of any one of embodiments 90 or 104-155, the system of any one of embodiments 92 or 104-155, or the cell of any one of embodiments 96-97 or 100-155, wherein the second cytokine peptide comprises an amino acid sequence that is at least 75%, 80%, 85%, 90%, 95%, 98%, 99%, or 100% identical to any of the sequences set forth in SEQ ID NOs: 100-107, or 109-112.

Embodiment 172. The nucleic acid molecule of any one of embodiments 90 or 104-155, the system of any one of embodiments 92 or 104-155, or the cell of any one of embodiments 96-97 or 100-155, wherein the second cytokine peptide is encoded by a nucleic acid sequence that is at least 75%, 80%, 85%, 90%, 95%, 98%, 99%, or 100% identical to any of the sequences set forth in SEQ ID NOs: 150-157 or 159-164.

Embodiment 173. The nucleic acid molecule of any one of embodiments 91 or 104-155, the system of any one of embodiments 93 or 104-155, or the cell of any one of embodiments 96 or 98-155, wherein the second cytokine peptide comprises an amino acid sequence that is at least 75%, 80%, 85%, 90%, 95%, 98%, 99%, or 100% identical to any of the sequences set forth in SEQ ID NOs: 100-105 or 107-112.

Embodiment 174. The nucleic acid molecule of any one of embodiments 91 or 104-155, the system of any one of embodiments 93 or 104-155, or the cell of any one of embodiments 96 or 98-155, wherein the second cytokine peptide is encoded by a nucleic acid sequence that is at least 75%, 80%, 85%, 90%, 95%, 98%, 99%, or 100% identical to any of the sequences set forth in SEQ ID NOs: 150-155 or 157-164.

Embodiment 175. The nucleic acid molecule of any one of embodiments 90-91 or 104-174, the system of any one of embodiments 92-93 or 104-174, or the cell of any one of embodiments 96-174, wherein the first cytokine peptide comprises at least a portion of IL-2 or a variant thereof, and the second cytokine peptide comprises at least a portion of IL-7 or a variant thereof.

Embodiment 176. The nucleic acid molecule of any one of embodiments 90-91 or 104-174, the system of any one of embodiments 92-93 or 104-174, or the cell of any one of embodiments 96-174, wherein the first cytokine peptide comprises at least a portion of IL-2 or a variant thereof, and the second cytokine peptide comprises at least a portion of IL-12 or a variant thereof.

Embodiment 177. The nucleic acid molecule of any one of embodiments 90-91 or 104-174, the system of any one of embodiments 92-93 or 104-174, or the cell of any one of embodiments 96-174, wherein the first cytokine peptide comprises at least a portion of IL-2 or a variant thereof, and the second cytokine peptide comprises at least a portion of IL-15 or a variant thereof.

Embodiment 178. The nucleic acid molecule of any one of embodiments 91 or 104-174, the system of any one of embodiments 93 or 104-174, or the cell of any one of embodiments 96 or 98-174, wherein the first cytokine peptide comprises at least a portion of IL-2 or a variant thereof, and the second cytokine peptide comprises at least a portion of IL-21 or a variant thereof.

Embodiment 179. The nucleic acid molecule of any one of embodiments 90-91 or 104-174, the system of any one of embodiments 92-93 or 104-174, or the cell of any one of embodiments 96-174, wherein the first cytokine peptide comprises at least a portion of IL-2 or a variant thereof, and the second cytokine peptide comprises at least a portion of IL-12p40 or a variant thereof.

Embodiment 180. The nucleic acid molecule of any one of embodiments 90-91 or 104-174, the system of any one of embodiments 92-93 or 104-174, or the cell of any one of embodiments 96-174, wherein the first cytokine peptide comprises at least a portion of IL-2 or a variant thereof, and the second cytokine peptide comprises at least a portion of IL-23p19 or a variant thereof.

Embodiment 181. The nucleic acid molecule of any one of embodiments 90-91 or 104-174, the system of any one of embodiments 92-93 or 104-174, or the cell of any one of embodiments 96-174, wherein the first cytokine peptide comprises at least a portion of IL-7 or a variant thereof, and the second cytokine peptide comprises at least a portion of IL-12 or a variant thereof.

Embodiment 182. The nucleic acid molecule of any one of embodiments 90 or 104-174, the system of any one of embodiments 92 or 104-174, or the cell of any one of embodiments 96-97 or 100-174, wherein the first cytokine peptide comprises at least a portion of IL-7 or a variant thereof, and the second cytokine peptide comprises at least a portion of IL-15 or a variant thereof.

Embodiment 183. The nucleic acid molecule of any one of embodiments 91 or 104-174, the system of any one of embodiments 93 or 104-174, or the cell of any one of embodiments 96 or 98-174, wherein the first cytokine peptide comprises at least a portion of IL-7 or a variant thereof, and the second cytokine peptide comprises at least a portion of IL-21 or a variant thereof.

Embodiment 184. The nucleic acid molecule of any one of embodiments 90-91 or 104-174, the system of any one of embodiments 92-93 or 104-174, or the cell of any one of embodiments 96-174, wherein the first cytokine peptide comprises at least a portion of IL-7 or a variant thereof, and the second cytokine peptide comprises at least a portion of IL-12p40 or a variant thereof.

Embodiment 185. The nucleic acid molecule of any one of embodiments 90-91 or 104-174, the system of any one of embodiments 92-93 or 104-174, or the cell of any one of embodiments 96-174, wherein the first cytokine peptide comprises at least a portion of IL-7 or a variant thereof, and the second cytokine peptide comprises at least a portion of IL-23p19 or a variant thereof.

Embodiment 186. The nucleic acid molecule of any one of embodiments 90 or 104-174, the system of any one of embodiments 92 or 104-174, or the cell of any one of embodiments 96-97 or 100-174, wherein the first cytokine peptide comprises at least a portion of IL-12 or a variant thereof, and the second cytokine peptide comprises at least a portion of IL-15 or a variant thereof.

Embodiment 187. The nucleic acid molecule of any one of embodiments 91 or 104-174, the system of any one of embodiments 93 or 104-174, or the cell of any one of embodiments 96 or 98-174, wherein the first cytokine peptide comprises at least a portion of IL-12 or a variant thereof, and the second cytokine peptide comprises at least a portion of IL-21 or a variant thereof.

Embodiment 188. The nucleic acid molecule of any one of embodiments 90-91 or 104-174, the system of any one of embodiments 92-93 or 104-174, or the cell of any one of embodiments 96-174, wherein the first cytokine peptide comprises at least a portion of IL-12 or a variant thereof, and the second cytokine peptide comprises at least a portion of IL-12p40 or a variant thereof.

Embodiment 189. The nucleic acid molecule of any one of embodiments 90-91 or 104-174, the system of any one of embodiments 92-93 or 104-174, or the cell of any one of embodiments 96-174, wherein the first cytokine peptide comprises at least a portion of IL-12 or a variant thereof, and the second cytokine peptide comprises at least a portion of IL-23p19 or a variant thereof.

Embodiment 190. The nucleic acid molecule of any one of embodiments 90 or 104-174, the system of any one of embodiments 92 or 104-174, or the cell of any one of embodiments 96-97 or 100-174, wherein the first cytokine peptide comprises at least a portion of IL-15 or a variant thereof, and the second cytokine peptide comprises at least a portion of IL-12p40 or a variant thereof.

Embodiment 191. The nucleic acid molecule of any one of embodiments 90 or 104-174, the system of any one of embodiments 92 or 104-174, or the cell of any one of embodiments 96-97 or 100-174, wherein the first cytokine peptide comprises at least a portion of IL-15 or a variant thereof, and the second cytokine peptide comprises at least a portion of IL-23p19 or a variant thereof.

Embodiment 192. The nucleic acid molecule of any one of embodiments 91 or 104-174, the system of any one of embodiments 93 or 104-174, or the cell of any one of embodiments 96 or 98-174, wherein the first cytokine peptide comprises at least a portion of IL-21 or a variant thereof, and the second cytokine peptide comprises at least a portion of IL-12p40 or a variant thereof.

Embodiment 193. The nucleic acid molecule of any one of embodiments 91 or 104-174, the system of any one of embodiments 93 or 104-174, or the cell of any one of embodiments 96 or 98-174, wherein the first cytokine peptide comprises at least a portion of IL-21 or a variant thereof, and the second cytokine peptide comprises at least a portion of IL-23p19 or a variant thereof.

Embodiment 194. The nucleic acid molecule of any one of embodiments 90-91 or 104-174, the system of any one of embodiments 92-93 or 104-174, or the cell of any one of embodiments 96-174, wherein the first cytokine peptide comprises at least a portion of IL-12p40 or a variant thereof, and the second cytokine peptide comprises at least a portion of IL-23p19 or a variant thereof.

Embodiment 195. The nucleic acid molecule of any one of embodiments 90, 91, 94, or 104-194, the system of any one of embodiments 92, 93, 95, or 104-194, or the cell of any one of embodiments 96-194, wherein the variant of the first cytokine peptide and the second cytokine peptide comprises an alteration, substitution, deletion, addition, or chemical modification of one or more amino acids, one or more unnatural amino acids, or any combination thereof.

Embodiment 196. The nucleic acid molecule of any one of embodiments 90, 91, 94, or 104-195, the system of any one of embodiments 92, 93, 95, or 104-195, or the cell of any one of embodiments 96-195, wherein the nucleic acid molecule, the system, or the cell further comprises a targeting sequence encoding a targeting moiety, and wherein the targeting moiety comprises a chimeric antigen receptor, a T cell receptor, a B cell receptor, or any combination thereof.

Embodiment 197. The nucleic acid molecule, the system, or the cell of embodiment 196, wherein the targeting sequence and the first nucleic acid sequence are under control of two different promoters.

Embodiment 198. The nucleic acid molecule, the system, or the cell of embodiment 196, wherein the targeting sequence and the first nucleic acid sequence are under control of a same promoter.

Embodiment 199. The nucleic acid molecule, the system, or the cell of any one of embodiments 196-198, wherein the targeting sequence and the second nucleic acid sequence are under control of two different promoters.

Embodiment 200. The nucleic acid molecule, the system, or the cell of any one of embodiments 196-198, wherein the targeting sequence and the second nucleic acid sequence are under control of a same promoter.

Embodiment 201. The nucleic acid molecule, the system, or the cell of any one of embodiments 196-200, wherein the targeting sequence encodes a chimeric antigen receptor (CAR).

Embodiment 202. The nucleic acid molecule, the system, or the cell of embodiment 201, wherein the chimeric antigen receptor (CAR) comprises a ligand binding domain, and wherein the ligand binding domain targets CD19, CD20, CD22, CD30, CD33, CD38, CD70, CD123, CD138, CD171, CD5, CD7, MUC1, AFP, CEA, PSCA, PSMA, HER2, EGFR, IL13Ralpha2, GD2, NKG2D, EGFTvIII, CS1, CCL1, BCMA, Mesothelin, ROR1, FLT3, FAP, TAG72, CD38, CD44v6, CEA, EPCAM, B7H3, KIT, IL-13Ralpha2, PRSS21, VEGR2, LewisY, CD24, PDGFR-beta, SSEA-4, AFP, NCAM, Claudin18.2, GPC3, GM3, TGS5, HMWMAA, TEM7R, CLDN6, GPRC5D, CXORF61, CD97, CD179a, ALK, PLAC1, GloboH, NY-BR-1, UPK2, HAVCR1, ADRB3, PANX3, GPR20, LY6K, OR51E2, TRAP, WT1, NY-ESO-1, LAGE-1a, or MAGE-A1.

Embodiment 203. The nucleic acid molecule, the system, or the cell of embodiment 202, wherein the ligand binding domain targets CD19.

Embodiment 204. The nucleic acid molecule, the system, or the cell of any one of embodiments 196-203, wherein the targeting sequence encodes CAR19.

Embodiment 205. The nucleic acid molecule, the system, or the cell of any one of embodiments 201-204, wherein the CAR comprises an amino acid sequence that is at least 75%, 80%, 85%, 90%, 95%, 98%, 99%, or 100% identical to any of the sequences set forth in SEQ ID NOs: 4 or 400-407.

Embodiment 206. The nucleic acid molecule, the system, or the cell of any one of embodiments 201-204, wherein the CAR is encoded by a nucleic acid sequence that is at least 75%, 80%, 85%, 90%, 95%, 98%, 99%, or 100% identical to any of the sequences set forth in SEQ ID NOs: 54 or 450-457.

Embodiment 207. The nucleic acid molecule, the system, or the cell of any one of embodiments 196-204, wherein the targeting sequence is linked to the first nucleic acid sequence or the second nucleic acid sequence via a nucleic acid sequence encoding a cleavable linker.

Embodiment 208. The nucleic acid molecule, the system, or the cell of embodiment 108 or 207, wherein the cleavable linker comprises P2A peptide, T2A peptide, E2A peptide, F2A peptide, or IRES peptide.

Embodiment 209. The nucleic acid molecule, the system, or the cell of embodiment 208, wherein the cleavable linker is encoded by a nucleic acid sequence having at least 75%, 80%, 85%, 90%, 95%, 98%, 99%, or 100% identical to any of the sequences set forth in SEQ ID NOs: 350-355.

Embodiment 210. The nucleic acid molecule, the system, or the cell of any one of embodiments 207-209, wherein the cleavable linker connects the CAR and the first signal peptide and/or the second signal peptide.

Embodiment 211. The nucleic acid molecule, the system, or the cell of any one of embodiments 207-210, wherein the cleavable linker connects the CAR and the first peptide anchor and/or the second peptide anchor.

Embodiment 212. The nucleic acid molecule, the system, or the cell of any one of embodiments 207-211, wherein the cleavable linker connects the CAR and the first non-peptide anchor attachment signal and/or the second non-peptide anchor attachment signal.

Embodiment 213. The nucleic acid molecule, the system, or the cell of any one of embodiments 108 or 207-212, wherein the cleavable linker comprises an amino acid sequence that is at least 75%, 80%, 85%, 90%, 95%, 98%, 99%, or 100% identical to any of the sequences set forth in SEQ ID NOs: 300-303.

Embodiment 214. The nucleic acid molecule of any one of embodiments 90, 91, 94, or 104-213, the system of any one of embodiments 92, 93, 95, or 104-213, or the cell of any one of embodiments 96-213, wherein each of the first polypeptide, the first protein, the second polypeptide, and the second protein independently further comprises a peptide linker.

Embodiment 215. The nucleic acid molecule, the system, or the cell of embodiment 214, wherein the peptide linker connects the first cytokine peptide to the first peptide anchor and/or connects the second cytokine peptide to the second peptide anchor.

Embodiment 216. The nucleic acid molecule, the system, or the cell of embodiment 214 or 215, wherein the peptide linker connects the first cytokine peptide to the first non-peptide anchor attachment signal and/or connects the second cytokine peptide to the second non-peptide anchor attachment signal.

Embodiment 217. The nucleic acid molecule, the system, or the cell of any one of embodiments 214-216, wherein the peptide linker connects the first peptide anchor and/or the second peptide anchor to the cleavable linker.

Embodiment 218. The nucleic acid molecule, the system, or the cell of any one of embodiments 214-217, wherein the peptide linker connects the first non-peptide anchor attachment signal and/or the second non-peptide anchor attachment signal to the cleavable linker.

Embodiment 219. The nucleic acid molecule, the system, or the cell of any one of embodiments 214-218, wherein the peptide linker comprises a GS linker, a Lr1 linker, or a Lr8 linker.

Embodiment 220. The nucleic acid molecule, the system, or the cell of any one of embodiments 214-219, wherein the peptide linker comprises an amino acid sequence that is at least 75%, 80%, 85%, 90%, 95%, 98%, 99%, or 100% identical to any of the sequences set forth in SEQ ID NOs: 500, 501, 504, 506, or 507, or the sequence of LE, AS, GSG, or EF.

Embodiment 221. The nucleic acid molecule, the system, or the cell of any one of embodiments 214-219, wherein the peptide linker is encoded by a nucleic acid sequence that is at least 75%, 80%, 85%, 90%, 95%, 98%, or 99%, or 100% identical to the sequence of ggctccggc, ggaagcgga, gagttc, the sequence of SEQ ID NO: 509, or the sequence of SEQ ID NO: 520.

Embodiment 222. The nucleic acid molecule of any one of embodiments 90, 91, 94, or 104-221, wherein the nucleic acid molecule is RNA.

Embodiment 223. The nucleic acid molecule of any one of embodiments 90, 91, 94, or 104-221, wherein the nucleic acid molecule is DNA.

Embodiment 224. The nucleic acid molecule of any one of embodiments 90, 91, 94, or 104-222, wherein the nucleic acid molecule is a linear RNA.

Embodiment 225. The nucleic acid molecule of any one of embodiments 90, 91, 94, or 104-222, wherein the nucleic acid molecule is a circular RNA.

Embodiment 226. The nucleic acid molecule of any one of embodiments 90, 91, 94, or 104-225, wherein the nucleic acid molecule is a vector.

Embodiment 227. The nucleic acid molecule of embodiment 226, wherein the vector is a viral vector.

Embodiment 228. The nucleic acid molecule of embodiment 226, wherein the vector is a retroviral vector, a lentiviral vector, an adenoviral vector, or an adeno-associated viral vector.

Embodiment 229. The system of any one of embodiments 92, 93, 95, or 104-221, wherein the targeting sequence and the first nucleic acid sequence are present in a same plasmid inside the system.

Embodiment 230. The system of any one of embodiments 92, 93, 95, or 104-221, wherein the targeting sequence and the first nucleic acid sequence are present in two different plasmids inside the system.

Embodiment 231. The system of any one of embodiments 92, 93, 95, 104-221, or 229-230, wherein the targeting sequence and the second nucleic acid sequence are present in a same plasmid inside the system.

Embodiment 232. The system of any one of embodiments 92, 93, 95, 104-221, or 229-230, wherein the targeting sequence and the second nucleic acid sequence are present in two different plasmids inside the system.

Embodiment 233. The system of any one of embodiments 92, 93, 95, 104-221, or 229-232, wherein the first nucleic acid sequence and the second nucleic acid sequence are present in a same plasmid inside the system.

Embodiment 234. The system of any one of embodiments 92, 93, 95, 104-221, or 229-232, wherein the first nucleic acid sequence and the second nucleic acid sequence are present in two different plasmids inside the system.

Embodiment 235. The cell of any one of embodiments 96-221, wherein the cell is a bacterial cell, yeast cell, or an insect cell.

Embodiment 236. The cell of any one of embodiments 96-221 or 235, wherein the cell is an immune cell or a tumor cell.

Embodiment 237. The cell of any one of embodiments 96-221 or 235-236, wherein the cell is an engineered immune cell.

Embodiment 238. The cell of any one of embodiments 96-221 or 235-236, wherein the cell is a T cell. Embodiment 239. The cell of any one of embodiments 96-221 or 235-236, wherein the cell is a tumor infiltrating lymphocyte (TIL).

Embodiment 240. The cell of embodiment 237, wherein the engineered immune cell is a natural killer (NK) cell.

Embodiment 241. The cell of any one of embodiments 96, 101-221, or 235-240, wherein the targeting sequence and the first nucleic acid sequence are present in a same plasmid inside the cell.

Embodiment 242. The cell of any one of embodiments 96, 101-221, or 235-240, wherein the targeting sequence and the first nucleic acid sequence are present in two different plasmids inside the cell.

Embodiment 243. The cell of any one of embodiments 96, 103-221, or 235-242, wherein the targeting sequence and the second nucleic acid sequence are present in a same plasmid inside the cell.

Embodiment 244. The cell of any one of embodiments 96, 103-221, or 235-242, wherein the targeting sequence and the second nucleic acid sequence are present in two different plasmids inside the cell.

Embodiment 245. The cell of any one of embodiments 96, 103-221, or 235-244, wherein the first nucleic acid sequence and the second nucleic acid sequence are present in a same plasmid inside the cell.

Embodiment 246. The cell of any one of embodiments 96, 103-221, or 235-244, wherein the first nucleic acid sequence and the second nucleic acid sequence are present in two different plasmids inside the cell.

Embodiment 247. The cell of any one of embodiments 96, 103-221, or 235-246, wherein the targeting sequence, the first nucleic acid sequence, and the second nucleic acid sequence are present in a genome of the cell.

Embodiment 248. The cell of any one of embodiments 236-247, wherein cytotoxicity of said immune cell is increased relative to a comparable immune cell that
(a) does not comprise both of: (1) the first nucleic acid sequence encodes the first polypeptide comprising the first cytokine peptide and (i) the first non-peptide anchor attachment signal or (ii) the first peptide anchor; and (2) the second nucleic acid sequence encodes the second polypeptide comprising the second cytokine peptide and (i) the second non-peptide anchor attachment signal or (ii) the second peptide anchor; or
(b) does not comprise both of: (1) the first protein comprising the first cytokine peptide and (i) the first non-peptide anchor or (ii) the first peptide anchor; and (2) the second protein comprising the second cytokine peptide and (i) the second non-peptide anchor or (ii) the second peptide anchor.

Embodiment 249. The cell of embodiment 248, wherein said increase in cytotoxicity of said immune cell is measured by an in vitro cytotoxicity assay described in Examples A-D.

Embodiment 250. The cell of embodiment 248, wherein said increase in cytotoxicity of said immune cell is measured in vitro or in vivo.

Embodiment 251. The cell of embodiment 248, wherein said increase in cytotoxicity of said immune cell is at least about 5%, 10%, 20%, 30%, 40%, 50% or more.

Embodiment 252. The cell of any one of embodiments 236-251, wherein a population of said immune cells proliferates for a longer period of time relative to a population of comparable immune cells that
(a) does not comprise the first nucleic acid sequence encodes the first polypeptide comprising the first cytokine peptide and (i) the first non-peptide anchor attachment signal or (ii) the first peptide anchor, and/or does not comprise the second nucleic acid sequence encodes the second polypeptide comprising the second cytokine peptide and (i) the second non-peptide anchor attachment signal or (ii) the second peptide anchor; or
(b) does not comprise the first protein comprising the first cytokine peptide and (i) the first non-peptide anchor or (ii) the first peptide anchor, and/or does not comprise the second protein comprising the second cytokine peptide and (i) the second non-peptide anchor or (ii) the second peptide anchor.

Embodiment 253. The cell of embodiment 252, wherein proliferation of said population of said immune cells is measured by an in vitro immune cell proliferation assay described in Examples A-E.

Embodiment 254. The cell of embodiment 252, wherein proliferation of said population of said immune cells is measured in vitro or in vivo.

Embodiment 255. The cell of embodiment 252, wherein proliferation of said population of said immune cells lasts for a period of time at least about 5%, 10%, 20%, 30%, 40%, 50%, 100%, 2 times, 3 times, 5 times, 10 times or longer than said population of said comparable immune cells.

Embodiment 256. A nucleic acid molecule comprising a first nucleic acid sequence, a second nucleic acid sequence, and a third nucleic acid sequence, wherein:
(a) the first nucleic acid sequence encodes a first polypeptide comprising a first cytokine peptide and (i) a first non-peptide anchor attachment signal or (ii) a first peptide anchor.
(b) the second nucleic acid sequence encodes a second polypeptide comprising a second cytokine peptide and (i) a second non-peptide anchor attachment signal or (ii) a second peptide anchor.
(c) the third nucleic acid sequence encodes a third polypeptide comprising a third cytokine peptide and (i) a third non-peptide anchor attachment signal or (ii) a third peptide anchor, wherein each of the first cytokine peptide, the second cytokine peptide, and the third cytokine peptide independently comprises at least a portion of IL-2, IL-4, IL-7, IL-9, IL-10, IL-12, IL-12p40, IL-15, IL-18, IL-21, IL-23, IL-27, IL-36γ, IL-23p19, or IL-1α or a variant thereof.

Embodiment 257. A system comprising a first nucleic acid sequence, a second nucleic acid sequence, and a third nucleic acid sequence, wherein:
(a) the first nucleic acid sequence encodes a first polypeptide comprising a first cytokine peptide and (i) a first non-peptide anchor attachment signal or (ii) a first peptide anchor.
(b) the second nucleic acid sequence encodes a second polypeptide comprising a second cytokine peptide and (i) a second non-peptide anchor attachment signal or (ii) a second peptide anchor.
(c) the third nucleic acid sequence encodes a third polypeptide comprising a third cytokine peptide and (i) a third non-peptide anchor attachment signal or (ii) a third peptide anchor,
wherein each of the first cytokine peptide, the second cytokine peptide, and the third cytokine peptide independently comprises at least a portion of IL-2, IL-4, IL-7, IL-9, IL-10, IL-12, IL-12p40, IL-15, IL-18, IL-21, IL-23, IL-27, IL-36γ, IL-23p19, or IL-1α or a variant thereof, and wherein the first nucleic acid sequence, the second nucleic acid sequence, and the third nucleic acid sequence are different.

Embodiment 258. A cell comprising the nucleic acid molecule of embodiment 256.

Embodiment 259. A cell comprising a first protein, a second protein, and a third protein, wherein:
(a) the first protein comprises a first cytokine peptide and (i) a first non-peptide anchor or (ii) a first peptide anchor.
(b) the second protein comprises a second cytokine peptide and (i) a second non-peptide anchor or (ii) a second peptide anchor.
(c) the third protein comprises a third cytokine peptide and (i) a third non-peptide anchor or (ii) a third peptide anchor,
wherein each of the first cytokine peptide, the second cytokine peptide, and the third cytokine peptide independently comprises at least a portion of IL-2, IL-4, IL-7, IL-9, IL-10, IL-12, IL-12p40, IL-15, IL-18, IL-21, IL-23, IL-27, IL-36γ, IL-23p19, or IL-1α or a variant thereof.

Embodiment 260. The cell of embodiment 259, wherein the first protein is processed from a first polypeptide comprising the first cytokine peptide and (i) a first non-peptide anchor attachment signal or (ii) the first peptide anchor, wherein the first non-peptide anchor attachment signal is replaced by the first non-peptide anchor during protein processing.

Embodiment 261. The cell of embodiment 260, wherein the first polypeptide is encoded by a first nucleic acid sequence.

Embodiment 262. The cell of any one of embodiments 259-261, wherein the second protein is processed from a second polypeptide comprising the second cytokine peptide and (i) a second non-peptide anchor attachment signal or (ii) the second peptide anchor, wherein the second non-peptide anchor attachment signal is replaced by the second non-peptide anchor during protein processing.

Embodiment 263. The cell of embodiment 262, wherein the second polypeptide is encoded by a second nucleic acid sequence.

Embodiment 264. The cell of any one of embodiments 259-263, wherein the third protein is processed from a third polypeptide comprising the third cytokine peptide and a third non-peptide anchor attachment signal or the third peptide anchor, wherein the third non-peptide anchor attachment signal is replaced by the third non-peptide anchor during protein processing.

Embodiment 265. The cell of embodiment 264, wherein the third polypeptide is encoded by a third nucleic acid sequence.

Embodiment 266. The nucleic acid molecule of embodiment 256, the system of embodiment 257, or the cell of any one of embodiments 263-265, wherein the second nucleic acid sequence and the first nucleic acid sequence are under control of a same promoter.

Embodiment 267. The nucleic acid molecule of embodiment 256, the system of embodiment 257, or the cell of any one of embodiments 263-265, wherein the second nucleic acid sequence and the first nucleic acid sequence are under control of two different promoters.

Embodiment 268. The nucleic acid molecule of any one of embodiments 256 or 266-267, the system of any one of embodiments 257 or 266-267, or the cell of any one of embodiments 265-267, wherein the second nucleic acid sequence and the third nucleic acid sequence are under control of a same promoter.

Embodiment 269. The nucleic acid molecule of any one of embodiments 256 or 266-267, the system of any one of embodiments 257 or 266-267, or the cell of any one of embodiments 265-267, wherein the second nucleic acid sequence and the third nucleic acid sequence are under control of two different promoters.

Embodiment 270. The nucleic acid molecule of any one of embodiments 256 or 266-269, the system of any one of embodiments 257 or 266-269, or the cell of any one of embodiments 265-269, wherein the first nucleic acid sequence and the third nucleic acid sequence are under control of a same promoter.

Embodiment 271. The nucleic acid molecule of any one of embodiments 256 or 266-269, the system of any one of embodiments 257 or 266-269, or the cell of any one of embodiments 265-269, wherein the first nucleic acid sequence and the third nucleic acid sequence are under control of two different promoters.

Embodiment 272. The nucleic acid molecule of any one of embodiments 256 or 266-271, the system of any one of embodiments 257 or 266-271, or the cell of any one of embodiments 265-271, wherein the first nucleic acid sequence, the second nucleic acid sequence, and the third nucleic acid sequence, are operably linked in a 5′ to 3′ direction.

Embodiment 273. The nucleic acid molecule of any one of embodiments 256 or 266-271, the system of any one of embodiments 257 or 266-271, or the cell of any one of embodiments 265-271, wherein the first nucleic acid sequence, the third nucleic acid sequence, and the second nucleic acid sequence are operably linked in a 5′ to 3′ direction.

Embodiment 274. The nucleic acid molecule of any one of embodiments 256 or 266-271, the system of any one of embodiments 257 or 266-271, or the cell of any one of embodiments 265-271, wherein the second nucleic acid sequence, the first nucleic acid sequence, and the third nucleic acid sequence, are operably linked in a 5′ to 3′ direction.

Embodiment 275. The nucleic acid molecule of any one of embodiments 256 or 266-271, the system of any one of embodiments 257 or 266-271, or the cell of any one of embodiments 265-271, wherein the second nucleic acid sequence, the third nucleic acid sequence, and the first nucleic acid sequence are operably linked in a 5′ to 3′ direction.

Embodiment 276. The nucleic acid molecule of any one of embodiments 256 or 266-271, the system of any one of embodiments 257 or 266-271, or the cell of any one of embodiments 265-271, wherein the third nucleic acid sequence, the second nucleic acid sequence, and the first nucleic acid sequence are operably linked in a 5′ to 3′ direction.

Embodiment 277. The nucleic acid molecule of any one of embodiments 256 or 266-271, the system of any one of embodiments 257 or 266-271, or the cell of any one of embodiments 265-271, wherein the third nucleic acid sequence, the first nucleic acid sequence, and the second nucleic acid sequence are operably linked in a 5′ to 3′ direction.

Embodiment 278. The nucleic acid molecule of any one of embodiments 256 or 266-277, the system of any one of embodiments 257 or 266-277, or the cell of any one of embodiments 265-277, wherein the first nucleic acid sequence and the second nucleic acid sequence are linked via a nucleic acid sequence encoding a cleavable linker.

Embodiment 279. The nucleic acid molecule of any one of embodiments 256 or 266-278, the system of any one of embodiments 257 or 266-278, or the cell of any one of embodiments 265-278, wherein the second nucleic acid sequence and the third nucleic acid sequence are linked via a nucleic acid sequence encoding a cleavable linker.

Embodiment 280. The nucleic acid molecule of any one of embodiments 256 or 266-279, the system of any one of embodiments 257 or 266-279, or the cell of any one of embodiments 265-279, wherein the first nucleic acid sequence and the third nucleic acid sequence are linked via a nucleic acid sequence encoding a cleavable linker.

Embodiment 281. The nucleic acid molecule of any one of embodiments 256 or 266-280, the system of any one of embodiments 257 or 266-280, or the cell of any one of embodiments 258-280, wherein the first protein or the first polypeptide comprises a first signal peptide.

Embodiment 282. The nucleic acid molecule, the system, or the cell of embodiment 281, wherein the first signal peptide, the first cytokine peptide, and (i) the first non-peptide anchor attachment signal or (ii) the first peptide anchor are operably linked in a direction from a N-terminal of the polypeptide to a C-terminal of the polypeptide.

Embodiment 283. The nucleic acid molecule of any one of embodiments 256 or 266-282, the system of any one of embodiments 257 or 266-282, or the cell of any one of embodiments 258-282, wherein the second protein or the second polypeptide comprises a second signal peptide.

Embodiment 284. The nucleic acid molecule, the system, or the cell of embodiment 283, wherein the second signal peptide, the second cytokine peptide, and (i) the second non-peptide anchor attachment signal or (ii) the second peptide anchor are operably linked in a direction from a N-terminal of the polypeptide to a C-terminal of the polypeptide.

Embodiment 285. The nucleic acid molecule of any one of embodiments 256 or 266-284, the system of any one of embodiments 257 or 266-284, or the cell of any one of embodiments 258-284, wherein the third protein or the third polypeptide comprises a third signal peptide.

Embodiment 286. The nucleic acid molecule, the system, or the cell of embodiment 285, wherein the third signal peptide, the third cytokine peptide, and (i) the third non-peptide anchor attachment signal or (ii) the third peptide anchor are operably linked in a direction from a N-terminal of the polypeptide to a C-terminal of the polypeptide.

Embodiment 287. The nucleic acid molecule, the system, or the cell of any one of embodiments 281-286, wherein each of the first signal peptide, the second signal peptide, and the third signal peptide independently comprises CD4 signal peptide, CD8α signal peptide, CD28 signal peptide, CD33 signal peptide, CD137 (4-1BB) signal peptide, IL-2 signal peptide, IgE signal peptide, IgG1 signal peptide. GM-CSF signal peptide, HLA-A signal peptide, HLA signal peptide, TCR signal peptide, or β2M signal peptide, or a combination thereof.

Embodiment 288. The nucleic acid molecule, the system, or the cell of any one of embodiments 281-286, wherein each of the first signal peptide, the second signal peptide, and the third signal peptide independently comprises an amino acid sequence that is at least 75%, 80%, 85%, 90%, 95%, 98%, 99%, or 100% identical to any of the sequences set forth in SEQ ID NOs: 2-7.

Embodiment 289. The nucleic acid molecule, the system, or the cell of any one of embodiments 281-286, wherein each of the first signal peptide, the second signal peptide, and the third signal peptide is independently encoded by a nucleic acid sequence that is at least 75%, 80%, 85%, 90%, 95%, 98%, 99%, or 100% identical to any of the sequences set forth in SEQ ID NOs: 52-57.

Embodiment 290. The nucleic acid molecule of any one of embodiments 256 or 266-289, the system of any one of embodiments 257 or 266-289, or the cell of any one of embodiments 260-289, wherein the first non-peptide anchor attachment signal comprises a glycolipid-attachment signal.

Embodiment 291. The nucleic acid molecule of any one of embodiments 256 or 266-290, the system of any one of embodiments 257 or 266-290, or the cell of any one of embodiments 262-290, wherein the second non-peptide anchor attachment signal comprises a glycolipid-attachment signal.

Embodiment 292. The nucleic acid molecule of any one of embodiments 256 or 266-291, the system of any one of embodiments 257 or 266-291, or the cell of any one of embodiments 264-291, wherein the third non-peptide anchor attachment signal comprises a glycolipid-attachment signal.

Embodiment 293. The nucleic acid molecule, the system, or the cell of any one of embodiments 290-292, wherein the glycolipid-attachment signal comprises a GPI-attachment signal.

Embodiment 294. The nucleic acid molecule, the system, or the cell of embodiment 293, wherein the GPI-attachment signal comprises an amino acid sequence that is at least 75%, 80%, 85%, 90%, 95%, 98%, 99%, or 100% identical to the sequence set forth in SEQ ID NO: 200.

Embodiment 295. The nucleic acid molecule, the system, or the cell of embodiment 293, wherein the GPI-attachment signal is encoded by a nucleic acid sequence that is at least 75%, 80%, 85%, 90%, 95%, 98%, 99%, or 100% identical to any of the sequences set forth in SEQ ID NOs: 250-252.

Embodiment 296. The nucleic acid molecule of any one of embodiments 256 or 266-295, the system of any one of embodiments 257 or 266-295, or the cell of any one of embodiments 258-295, wherein each of the first peptide anchor, the second peptide anchor, and the third peptide anchor independently comprises a transmembrane peptide sequence.

Embodiment 297. The nucleic acid molecule, the system, or the cell of embodiment 296, wherein the transmembrane peptide sequence comprises a B7-1 transmembrane amino acid sequence, a B7-2 transmembrane amino acid sequence. B7-H1 transmembrane amino acid sequence. B7-H3 transmembrane amino acid sequence, tumor necrosis factor receptor 2 (TNFR2) transmembrane amino acid sequence, a CD8α transmembrane amino acid sequence, a CD28 transmembrane amino acid sequence, a CD3ζ transmembrane amino acid sequence, a CTLA-4 (CD152) transmembrane amino acid sequence, or a PD-L1 transmembrane amino acid sequence, or any fragments or variants thereof.

Embodiment 298. The nucleic acid molecule, the system, or the cell of embodiment 296 or 297, wherein the transmembrane peptide sequence comprises an amino acid sequence that is at least 75%, 80%, 85%, 90%, 95%, 98%, 99%, or 100% identical to any of the sequences set forth in SEQ ID NOs: 203-204.

Embodiment 299. The nucleic acid molecule, the system, or the cell of embodiment 296 or 297, wherein the transmembrane peptide sequence is encoded by a nucleic acid sequence that is at least 75%, 80%, 85%, 90%, 95%, 98%, 99%, or 100% identical to any of the sequences set forth in SEQ ID NOs: 253-255.

Embodiment 300. The nucleic acid molecule of any one of embodiments 256 or 266-299, the system of any one of embodiments 257 or 266-299, or the cell of any one of embodiments 258-299, wherein the first cytokine peptide comprises at least a portion of IL-2 or a variant thereof.

Embodiment 301. The nucleic acid molecule of any one of embodiments 256 or 266-299, the system of any one of embodiments 257 or 266-299, or the cell of any one of embodiments 258-299, wherein the first cytokine peptide comprises at least a portion of IL-4 or a variant thereof.

Embodiment 302. The nucleic acid molecule of any one of embodiments 256 or 266-299, the system of any one of embodiments 257 or 266-299, or the cell of any one of embodiments 258-299, wherein the first cytokine peptide comprises at least a portion of IL-7 or a variant thereof.

Embodiment 303. The nucleic acid molecule of any one of embodiments 256 or 266-299, the system of any one of embodiments 257 or 266-299, or the cell of any one of embodiments 258-299, wherein the first cytokine peptide comprises at least a portion of IL-9 or a variant thereof.

Embodiment 304. The nucleic acid molecule of any one of embodiments 256 or 266-299, the system of any one of embodiments 257 or 266-299, or the cell of any one of embodiments 258-299, wherein the first cytokine peptide comprises at least a portion of IL-10 or a variant thereof.

Embodiment 305. The nucleic acid molecule of any one of embodiments 256 or 266-299, the system of any one of embodiments 257 or 266-299, or the cell of any one of embodiments 258-299, wherein the first cytokine peptide comprises at least a portion of IL-12 or a variant thereof.

Embodiment 306. The nucleic acid molecule of any one of embodiments 256 or 266-299, the system of any one of embodiments 257 or 266-299, or the cell of any one of embodiments 258-299, wherein the first cytokine peptide comprises at least a portion of IL-12p40 or a variant thereof.

Embodiment 307. The nucleic acid molecule of any one of embodiments 256 or 266-299, the system of any one of embodiments 257 or 266-299, or the cell of any one of embodiments 258-299, wherein the first cytokine peptide comprises at least a portion of IL-18 or a variant thereof.

Embodiment 308. The nucleic acid molecule of any one of embodiments 256 or 266-299, the system of any one of embodiments 257 or 266-299, or the cell of any one of embodiments 258-299, wherein the first cytokine peptide comprises at least a portion of IL-23 or a variant thereof.

Embodiment 309. The nucleic acid molecule of any one of embodiments 256 or 266-299, the system of any one of embodiments 257 or 266-299, or the cell of any one of embodiments 258-299, wherein the first cytokine peptide comprises at least a portion of IL-27 or a variant thereof.

Embodiment 310. The nucleic acid molecule of any one of embodiments 256 or 266-299, the system of any one of embodiments 257 or 266-299, or the cell of any one of embodiments 258-299, wherein the first cytokine peptide comprises at least a portion of IL-36γ or a variant thereof.

Embodiment 311. The nucleic acid molecule of any one of embodiments 256 or 266-299, the system of any one of embodiments 257 or 266-299, or the cell of any one of embodiments 258-299, wherein the first cytokine peptide comprises at least a portion of IL-23p19 or a variant thereof.

Embodiment 312. The nucleic acid molecule of any one of embodiments 256 or 266-299, the system of any one of embodiments 257 or 266-299, or the cell of any one of embodiments 258-299, wherein the first cytokine peptide comprises at least a portion of IL-1α or a variant thereof.

Embodiment 313. The nucleic acid molecule of any one of embodiments 256 or 266-299, the system of any one of embodiments 257 or 266-299, or the cell of any one of embodiments 258-299, wherein the first cytokine peptide comprises at least a portion of IL-15 or a variant thereof.

Embodiment 314. The nucleic acid molecule of any one of embodiments 256 or 266-299, the system of any one of embodiments 257 or 266-299, or the cell of any one of embodiments 258-299, wherein the first cytokine peptide comprises at least a portion of IL-21 or a variant thereof.

Embodiment 315. The nucleic acid molecule of any one of embodiments 256 or 266-299, the system of any one of embodiments 257 or 266-299, or the cell of any one of embodiments 258-299, wherein the first cytokine peptide comprises an amino acid sequence that is at least 75%, 80%, 85%, 90%, 95%, 98%, 99%, or 100% identical to any of the sequences set forth in SEQ ID NOs: 100-112.

Embodiment 316. The nucleic acid molecule of any one of embodiments 256 or 266-299, the system of any one of embodiments 257 or 266-299, or the cell of any one of embodiments 258-299, wherein the first cytokine peptide is encoded by a nucleic acid sequence that is at least 75%, 80%, 85%, 90%, 95%, 98%, 99%, or 100% identical to any of the sequences set forth in SEQ ID NOs: 150-164.

Embodiment 317. The nucleic acid molecule of any one of embodiments 256 or 266-316, the system of any one of embodiments 257 or 266-316, or the cell of any one of embodiments 258-316, wherein the second cytokine peptide comprises at least a portion of IL-2 or a variant thereof.

Embodiment 318. The nucleic acid molecule of any one of embodiments 256 or 266-316, the system of any one of embodiments 257 or 266-316, or the cell of any one of embodiments 258-316, wherein the second cytokine peptide comprises at least a portion of IL-4 or a variant thereof.

Embodiment 319. The nucleic acid molecule of any one of embodiments 256 or 266-316, the system of any one of embodiments 257 or 266-316, or the cell of any one of embodiments 258-316, wherein the second cytokine peptide comprises at least a portion of IL-7 or a variant thereof.

Embodiment 320. The nucleic acid molecule of any one of embodiments 256 or 266-316, the system of any one of embodiments 257 or 266-316, or the cell of any one of embodiments 258-316, wherein the second cytokine peptide comprises at least a portion of IL-9 or a variant thereof.

Embodiment 321. The nucleic acid molecule of any one of embodiments 256 or 266-316, the system of any one of embodiments 257 or 266-316, or the cell of any one of embodiments 258-316, wherein the second cytokine peptide comprises at least a portion of IL-10 or a variant thereof.

Embodiment 322. The nucleic acid molecule of any one of embodiments 256 or 266-316, the system of any one of embodiments 257 or 266-316, or the cell of any one of embodiments 258-316, wherein the second cytokine peptide comprises at least a portion of IL-12 or a variant thereof.

Embodiment 323. The nucleic acid molecule of any one of embodiments 256 or 266-316, the system of any one of embodiments 257 or 266-316, or the cell of any one of embodiments 258-316, wherein the second cytokine peptide comprises at least a portion of IL-12p40 or a variant thereof.

Embodiment 324. The nucleic acid molecule of any one of embodiments 256 or 266-316, the system of any one of embodiments 257 or 266-316, or the cell of any one of embodiments 258-316, wherein the second cytokine peptide comprises at least a portion of IL-18 or a variant thereof.

Embodiment 325. The nucleic acid molecule of any one of embodiments 256 or 266-316, the system of any one of embodiments 257 or 266-316, or the cell of any one of embodiments 258-316, wherein the second cytokine peptide comprises at least a portion of IL-23 or a variant thereof.

Embodiment 326. The nucleic acid molecule of any one of embodiments 256 or 266-316, the system of any one of embodiments 257 or 266-316, or the cell of any one of embodiments 258-316, wherein the second cytokine peptide comprises at least a portion of IL-27 or a variant thereof.

Embodiment 327. The nucleic acid molecule of any one of embodiments 256 or 266-316, the system of any one of embodiments 257 or 266-316, or the cell of any one of embodiments 258-316, wherein the second cytokine peptide comprises at least a portion of IL-36γ or a variant thereof.

Embodiment 328. The nucleic acid molecule of any one of embodiments 256 or 266-316, the system of any one of embodiments 257 or 266-316, or the cell of any one of embodiments 258-316, wherein the second cytokine peptide comprises at least a portion of IL-23p19 or a variant thereof.

Embodiment 329. The nucleic acid molecule of any one of embodiments 256 or 266-316, the system of any one of embodiments 257 or 266-316, or the cell of any one of embodiments 258-316, wherein the second cytokine peptide comprises at least a portion of IL-1α or a variant thereof.

Embodiment 330. The nucleic acid molecule of any one of embodiments 256 or 266-316, the system of any one of embodiments 257 or 266-316, or the cell of any one of embodiments 258-316, wherein the second cytokine peptide comprises at least a portion of IL-15 or a variant thereof.

Embodiment 331. The nucleic acid molecule of any one of embodiments 256 or 266-316, the system of any one of embodiments 257 or 266-316, or the cell of any one of embodiments 258-316, wherein the second cytokine peptide comprises at least a portion of IL-21 or a variant thereof.

Embodiment 332. The nucleic acid molecule of any one of embodiments 256 or 266-316, the system of any one of embodiments 257 or 266-316, or the cell of any one of embodiments 258-316, wherein the second cytokine peptide comprises an amino acid sequence that is at least 75%, 80%, 85%, 90%, 95%, 98%, 99%, or 100% identical to any of the sequences set forth in SEQ ID NOs: 100-112.

Embodiment 333. The nucleic acid molecule of any one of embodiments 256 or 266-316, the system of any one of embodiments 257 or 266-316, or the cell of any one of embodiments 258-316, wherein the second cytokine peptide is encoded by a nucleic acid sequence that is at least 75%, 80%, 85%, 90%, 95%, 98%, 99%, or 100% identical to any of the sequences set forth in SEQ ID NOs: 150-164.

Embodiment 334. The nucleic acid molecule of any one of embodiments 256 or 266-333, the system of any one of embodiments 257 or 266-333, or the cell of any one of embodiments 258-333, wherein the third cytokine peptide comprises at least a portion of IL-2 or a variant thereof.

Embodiment 335. The nucleic acid molecule of any one of embodiments 256 or 266-333, the system of any one of embodiments 257 or 266-333, or the cell of any one of embodiments 258-333, wherein the third cytokine peptide comprises at least a portion of IL-4 or a variant thereof.

Embodiment 336. The nucleic acid molecule of any one of embodiments 256 or 266-333, the system of any one of embodiments 257 or 266-333, or the cell of any one of embodiments 258-333, wherein the third cytokine peptide comprises at least a portion of IL-7 or a variant thereof.

Embodiment 337. The nucleic acid molecule of any one of embodiments 256 or 266-333, the system of any one of embodiments 257 or 266-333, or the cell of any one of embodiments 258-333, wherein the third cytokine peptide comprises at least a portion of IL-9 or a variant thereof.

Embodiment 338. The nucleic acid molecule of any one of embodiments 256 or 266-333, the system of any one of embodiments 257 or 266-333, or the cell of any one of embodiments 258-333, wherein the third cytokine peptide comprises at least a portion of IL-10 or a variant thereof.

Embodiment 339. The nucleic acid molecule of any one of embodiments 256 or 266-333, the system of any one of embodiments 257 or 266-333, or the cell of any one of embodiments 258-333, wherein the third cytokine peptide comprises at least a portion of IL-12 or a variant thereof.

Embodiment 340. The nucleic acid molecule of any one of embodiments 256 or 266-333, the system of any one of embodiments 257 or 266-333, or the cell of any one of embodiments 258-333, wherein the third cytokine peptide comprises at least a portion of IL-12p40 or a variant thereof.

Embodiment 341. The nucleic acid molecule of any one of embodiments 256 or 266-333, the system of any one of embodiments 257 or 266-333, or the cell of any one of embodiments 258-333, wherein the third cytokine peptide comprises at least a portion of IL-18 or a variant thereof.

Embodiment 342. The nucleic acid molecule of any one of embodiments 256 or 266-333, the system of any one of embodiments 257 or 266-333, or the cell of any one of embodiments 258-333, wherein the third cytokine peptide comprises at least a portion of IL-23 or a variant thereof.

Embodiment 343. The nucleic acid molecule of any one of embodiments 256 or 266-333, the system of any one of embodiments 257 or 266-333, or the cell of any one of embodiments 258-333, wherein the third cytokine peptide comprises at least a portion of IL-27 or a variant thereof.

Embodiment 344. The nucleic acid molecule of any one of embodiments 256 or 266-333, the system of any one of embodiments 257 or 266-333, or the cell of any one of embodiments 258-333, wherein the third cytokine peptide comprises at least a portion of IL-36γ or a variant thereof.

Embodiment 345. The nucleic acid molecule of any one of embodiments 256 or 266-333, the system of any one of embodiments 257 or 266-333, or the cell of any one of embodiments 258-333, wherein the third cytokine peptide comprises at least a portion of IL-23p19 or a variant thereof.

Embodiment 346. The nucleic acid molecule of any one of embodiments 256 or 266-333, the system of any one of embodiments 257 or 266-333, or the cell of any one of embodiments 258-333, wherein the third cytokine peptide comprises at least a portion of IL-1α or a variant thereof.

Embodiment 347. The nucleic acid molecule of any one of embodiments 256 or 266-333, the system of any one of embodiments 257 or 266-333, or the cell of any one of embodiments 258-333, wherein the third cytokine peptide comprises at least a portion of IL-15 or a variant thereof.

Embodiment 348. The nucleic acid molecule of any one of embodiments 256 or 266-333, the system of any one of embodiments 257 or 266-333, or the cell of any one of embodiments 258-333, wherein the third cytokine peptide comprises at least a portion of IL-21 or a variant thereof.

Embodiment 349. The nucleic acid molecule of any one of embodiments 256 or 266-333, the system of any one of embodiments 257 or 266-333, or the cell of any one of embodiments 258-333, wherein the third cytokine peptide comprises an amino acid sequence that is at least 75%, 80%, 85%, 90%, 95%, 98%, 99%, or 100% identical to any of the sequences set forth in SEQ ID NOs: 100-112.

Embodiment 350. The nucleic acid molecule of any one of embodiments 256 or 266-333, the system of any one of embodiments 257 or 266-333, or the cell of any one of embodiments 258-333, wherein the third cytokine peptide is encoded by a nucleic acid sequence that is at least 75%, 80%, 85%, 90%, 95%, 98%, 99%, or 100% identical to any of the sequences set forth in SEQ ID NOs: 150-164.

Embodiment 351. The nucleic acid molecule of any one of embodiments 256 or 266-350, the system of any one of embodiments 257 or 266-350, or the cell of any one of embodiments 258-350, wherein the first cytokine peptide comprises at least a portion of IL-2 or a variant thereof, the second cytokine peptide comprises at least a portion of IL-7 or a variant thereof, and the third cytokine peptide comprises at least a portion of IL-12 or a variant thereof.

Embodiment 352. The nucleic acid molecule of any one of embodiments 256 or 266-350, the system of any one of embodiments 257 or 266-350, or the cell of any one of embodiments 258-350, wherein the first cytokine peptide comprises at least a portion of IL-2 or a variant thereof, the second cytokine peptide comprises at least a portion of IL-7 or a variant thereof, and the third cytokine peptide comprises at least a portion of IL-15 or a variant thereof.

Embodiment 353. The nucleic acid molecule of any one of embodiments 256 or 266-350, the system of any one of embodiments 257 or 266-350, or the cell of any one of embodiments 258-350, wherein the first cytokine peptide comprises at least a portion of IL-2 or a variant thereof, the second cytokine peptide comprises at least a portion of IL-7 or a variant thereof, and the third cytokine peptide comprises at least a portion of IL-21 or a variant thereof.

Embodiment 354. The nucleic acid molecule of any one of embodiments 256 or 266-350, the system of any one of embodiments 257 or 266-350, or the cell of any one of embodiments 258-350, wherein the first cytokine peptide comprises at least a portion of IL-2 or a variant thereof, the second cytokine peptide comprises at least a portion of IL-7 or a variant thereof, and the third cytokine peptide comprises at least a portion of IL-12p40 or a variant thereof.

Embodiment 355. The nucleic acid molecule of any one of embodiments 256 or 266-350, the system of any one of embodiments 257 or 266-350, or the cell of any one of embodiments 258-350, wherein the first cytokine peptide comprises at least a portion of IL-2 or a variant thereof, the second cytokine peptide comprises at least a portion of IL-7 or a variant thereof, and the third cytokine peptide comprises at least a portion of IL-23p19 or a variant thereof.

Embodiment 356. The nucleic acid molecule of any one of embodiments 256 or 266-350, the system of any one of embodiments 257 or 266-350, or the cell of any one of embodiments 258-350, wherein the first cytokine peptide comprises at least a portion of IL-2 or a variant thereof, the second cytokine peptide comprises at least a portion of IL-12 or a variant thereof, and the third cytokine peptide comprises at least a portion of IL-15 or a variant thereof.

Embodiment 357. The nucleic acid molecule of any one of embodiments 256 or 266-350, the system of any one of embodiments 257 or 266-350, or the cell of any one of embodiments 258-350, wherein the first cytokine peptide comprises at least a portion of IL-2 or a variant thereof, the second cytokine peptide comprises at least a portion of IL-12 or a variant thereof, and the third cytokine peptide comprises at least a portion of IL-21 or a variant thereof.

Embodiment 358. The nucleic acid molecule of any one of embodiments 256 or 266-350, the system of any one of embodiments 257 or 266-350, or the cell of any one of embodiments 258-350, wherein the first cytokine peptide comprises at least a portion of IL-2 or a variant thereof, the second cytokine peptide comprises at least a portion of IL-12 or a variant thereof, and the third cytokine peptide comprises at least a portion of IL-12p40 or a variant thereof.

Embodiment 359. The nucleic acid molecule of any one of embodiments 256 or 266-350, the system of any one of embodiments 257 or 266-350, or the cell of any one of embodiments 258-350, wherein the first cytokine peptide comprises at least a portion of IL-2 or a variant thereof, the second cytokine peptide comprises at least a portion of IL-12 or a variant thereof, and the third cytokine peptide comprises at least a portion of IL-23p19 or a variant thereof.

Embodiment 360. The nucleic acid molecule of any one of embodiments 256 or 266-350, the system of any one of embodiments 257 or 266-350, or the cell of any one of embodiments 258-350, wherein the first cytokine peptide comprises at least a portion of IL-2 or a variant thereof, the second cytokine peptide comprises at least a portion of IL-15 or a variant thereof, and the third cytokine peptide comprises at least a portion of IL-21 or a variant thereof.

Embodiment 361. The nucleic acid molecule of any one of embodiments 256 or 266-350, the system of any one of embodiments 257 or 266-350), or the cell of any one of embodiments 258-350, wherein the first cytokine peptide comprises at least a portion of IL-2 or a variant thereof, the second cytokine peptide comprises at least a portion of IL-15 or a variant thereof, and the third cytokine peptide comprises at least a portion of IL-12p40 or a variant thereof.

Embodiment 362. The nucleic acid molecule of any one of embodiments 256 or 266-350, the system of any one of embodiments 257 or 266-350, or the cell of any one of embodiments 258-350, wherein the first cytokine peptide comprises at least a portion of IL-2 or a variant thereof, the second cytokine peptide comprises at least a portion of IL-15 or a variant thereof, and the third cytokine peptide comprises at least a portion of IL-23p19 or a variant thereof.

Embodiment 363. The nucleic acid molecule of any one of embodiments 256 or 266-350), the system of any one of embodiments 257 or 266-350), or the cell of any one of embodiments 258-350, wherein the first cytokine peptide comprises at least a portion of IL-2 or a variant thereof, the second cytokine peptide comprises at least a portion of IL-21 or a variant thereof, and the third cytokine peptide comprises at least a portion of IL-12p40 or a variant thereof.

Embodiment 364. The nucleic acid molecule of any one of embodiments 256 or 266-350), the system of any one of embodiments 257 or 266-350), or the cell of any one of embodiments 258-350, wherein the first cytokine peptide comprises at least a portion of IL-2 or a variant thereof, the second cytokine peptide comprises at least a portion of IL-21 or a variant thereof, and the third cytokine peptide comprises at least a portion of IL-23p19 or a variant thereof.

Embodiment 365. The nucleic acid molecule of any one of embodiments 256 or 266-350, the system of any one of embodiments 257 or 266-350), or the cell of any one of embodiments 258-350, wherein the first cytokine peptide comprises at least a portion of IL-2 or a variant thereof, the second cytokine peptide comprises at least a portion of IL-12p40 or a variant thereof, and the third cytokine peptide comprises at least a portion of IL-23p19 or a variant thereof.

Embodiment 366. The nucleic acid molecule of any one of embodiments 256 or 266-350, the system of any one of embodiments 257 or 266-350, or the cell of any one of embodiments 258-350, wherein the first cytokine peptide comprises at least a portion of IL-7 or a variant thereof, the second cytokine peptide comprises at least a portion of IL-12 or a variant thereof, and the third cytokine peptide comprises at least a portion of IL-15 or a variant thereof.

Embodiment 367. The nucleic acid molecule of any one of embodiments 256 or 266-350, the system of any one of embodiments 257 or 266-350, or the cell of any one of embodiments 258-350, wherein the first cytokine peptide comprises at least a portion of IL-7 or a variant thereof, the second cytokine peptide comprises at least a portion of IL-12 or a variant thereof, and the third cytokine peptide comprises at least a portion of IL-21 or a variant thereof.

Embodiment 368. The nucleic acid molecule of any one of embodiments 256 or 266-350, the system of any one of embodiments 257 or 266-350, or the cell of any one of embodiments 258-350, wherein the first cytokine peptide comprises at least a portion of IL-7 or a variant thereof, the second cytokine peptide comprises at least a portion of IL-12 or a variant thereof, and the third cytokine peptide comprises at least a portion of IL-12p40 or a variant thereof.

Embodiment 369. The nucleic acid molecule of any one of embodiments 256 or 266-350, the system of any one of embodiments 257 or 266-350, or the cell of any one of embodiments 258-350, wherein the first cytokine peptide comprises at least a portion of IL-7 or a variant thereof, the second cytokine peptide comprises at least a portion of IL-12 or a variant thereof, and the third cytokine peptide comprises at least a portion of IL-23p19 or a variant thereof.

Embodiment 370. The nucleic acid molecule of any one of embodiments 256 or 266-350, the system of any one of embodiments 257 or 266-350, or the cell of any one of embodiments 258-350, wherein the first cytokine peptide comprises at least a portion of IL-7 or a variant thereof, the second cytokine peptide comprises at least a portion of IL-15 or a variant thereof, and the third cytokine peptide comprises at least a portion of IL-21 or a variant thereof.

Embodiment 371. The nucleic acid molecule of any one of embodiments 256 or 266-350, the system of any one of embodiments 257 or 266-350, or the cell of any one of embodiments 258-350, wherein the first cytokine peptide comprises at least a portion of IL-7 or a variant thereof, the second cytokine peptide comprises at least a portion of IL-15 or a variant thereof, and the third cytokine peptide comprises at least a portion of IL-12p40 or a variant thereof.

Embodiment 372. The nucleic acid molecule of any one of embodiments 256 or 266-350, the system of any one of embodiments 257 or 266-350, or the cell of any one of embodiments 258-350, wherein the first cytokine peptide comprises at least a portion of IL-7 or a variant thereof, the second cytokine peptide comprises at least a portion of IL-15 or a variant thereof, and the third cytokine peptide comprises at least a portion of IL-23p19 or a variant thereof.

Embodiment 373. The nucleic acid molecule of any one of embodiments 256 or 266-350, the system of any one of embodiments 257 or 266-350, or the cell of any one of embodiments 258-350, wherein the first cytokine peptide comprises at least a portion of IL-7 or a variant thereof, the second cytokine peptide comprises at least a portion of IL-21 or a variant thereof, and the third cytokine peptide comprises at least a portion of IL-12p40 or a variant thereof.

Embodiment 374. The nucleic acid molecule of any one of embodiments 256 or 266-350, the system of any one of embodiments 257 or 266-350, or the cell of any one of embodiments 258-350, wherein the first cytokine peptide comprises at least a portion of IL-7 or a variant thereof, the second cytokine peptide comprises at least a portion of IL-21 or a variant thereof, and the third cytokine peptide comprises at least a portion of IL-23p19 or a variant thereof.

Embodiment 375. The nucleic acid molecule of any one of embodiments 256 or 266-350), the system of any one of embodiments 257 or 266-350, or the cell of any one of embodiments 258-350, wherein the first cytokine peptide comprises at least a portion of IL-7 or a variant thereof, the second cytokine peptide comprises at least a portion of IL-12p40 or a variant thereof, and the third cytokine peptide comprises at least a portion of IL-23p19 or a variant thereof.

Embodiment 376. The nucleic acid molecule of any one of embodiments 256 or 266-350, the system of any one of embodiments 257 or 266-350, or the cell of any one of embodiments 258-350, wherein the first cytokine peptide comprises at least a portion of IL-12 or a variant thereof, the second cytokine peptide comprises at least a portion of IL-15 or a variant thereof, and the third cytokine peptide comprises at least a portion of IL-21 or a variant thereof.

Embodiment 377. The nucleic acid molecule of any one of embodiments 256 or 266-350, the system of any one of embodiments 257 or 266-350), or the cell of any one of embodiments 258-350, wherein the first cytokine peptide comprises at least a portion of IL-12 or a variant thereof, the second cytokine peptide comprises at least a portion of IL-15 or a variant thereof, and the third cytokine peptide comprises at least a portion of IL-12p40 or a variant thereof.

Embodiment 378. The nucleic acid molecule of any one of embodiments 256 or 266-350), the system of any one of embodiments 257 or 266-350, or the cell of any one of embodiments 258-350, wherein the first cytokine peptide comprises at least a portion of IL-12 or a variant thereof, the second cytokine peptide comprises at least a portion of IL-15 or a variant thereof, and the third cytokine peptide comprises at least a portion of IL-23p19 or a variant thereof.

Embodiment 379. The nucleic acid molecule of any one of embodiments 256 or 266-350, the system of any one of embodiments 257 or 266-350), or the cell of any one of embodiments 258-350, wherein the first cytokine peptide comprises at least a portion of IL-12 or a variant thereof, the second cytokine peptide comprises at least a portion of IL-21 or a variant thereof, and the third cytokine peptide comprises at least a portion of IL-12p40 or a variant thereof.

Embodiment 380. The nucleic acid molecule of any one of embodiments 256 or 266-350, the system of any one of embodiments 257 or 266-350, or the cell of any one of embodiments 258-350, wherein the first cytokine peptide comprises at least a portion of IL-12 or a variant thereof, the second cytokine peptide comprises at least a portion of IL-21 or a variant thereof, and the third cytokine peptide comprises at least a portion of IL-23p19 or a variant thereof.

Embodiment 381. The nucleic acid molecule of any one of embodiments 256 or 266-350, the system of any one of embodiments 257 or 266-350, or the cell of any one of embodiments 258-350, wherein the first cytokine peptide comprises at least a portion of IL-12 or a variant thereof, the second cytokine peptide comprises at least a portion of IL-12p40 or a variant thereof, and the third cytokine peptide comprises at least a portion of IL-23p19 or a variant thereof.

Embodiment 382. The nucleic acid molecule of any one of embodiments 256 or 266-350, the system of any one of embodiments 257 or 266-350, or the cell of any one of embodiments 258-350, wherein the first cytokine peptide comprises at least a portion of IL-15 or a variant thereof, the second cytokine peptide comprises at least a portion of IL-21 or a variant thereof, and the third cytokine peptide comprises at least a portion of IL-12p40 or a variant thereof.

Embodiment 383. The nucleic acid molecule of any one of embodiments 256 or 266-350, the system of any one of embodiments 257 or 266-350, or the cell of any one of embodiments 258-350, wherein the first cytokine peptide comprises at least a portion of IL-15 or a variant thereof, the second cytokine peptide comprises at least a portion of IL-21 or a variant thereof, and the third cytokine peptide comprises at least a portion of IL-23p19 or a variant thereof.

Embodiment 384. The nucleic acid molecule of any one of embodiments 256 or 266-350, the system of any one of embodiments 257 or 266-350, or the cell of any one of embodiments 258-350, wherein the first cytokine peptide comprises at least a portion of IL-15 or a variant thereof, the second cytokine peptide comprises at least a portion of IL-12p40 or a variant thereof, and the third cytokine peptide comprises at least a portion of IL-23p19 or a variant thereof.

Embodiment 385. The nucleic acid molecule of any one of embodiments 256 or 266-350, the system of any one of embodiments 257 or 266-350, or the cell of any one of embodiments 258-350, wherein the first cytokine peptide comprises at least a portion of IL-21 or a variant thereof, the second cytokine peptide comprises at least a portion of IL-12p40 or a variant thereof, and the third cytokine peptide comprises at least a portion of IL-23p19 or a variant thereof.

Embodiment 386. The nucleic acid molecule of any one of embodiments 256 or 266-385, the system of any one of embodiments 257 or 266-385, or the cell of any one of embodiments 258-385, wherein the variant of the first cytokine peptide, the second cytokine peptide, and the third cytokine peptide comprises an alteration, substitution, deletion, addition, or chemical modification of one or more amino acids, one or more unnatural amino acids, or any combination thereof.

Embodiment 387. The nucleic acid molecule of any one of embodiments 256 or 266-386, the system of any one of embodiments 257 or 266-386, or the cell of any one of embodiments 258-386, wherein the nucleic acid molecule or the system or the cell further comprises a targeting sequence encoding a targeting moiety, wherein the targeting moiety comprises a chimeric antigen receptor, a T cell receptor, a B cell receptor, or any combination thereof.

Embodiment 388. The nucleic acid molecule, the system, or the cell of embodiment 387, wherein the targeting sequence and the first nucleic acid sequence are under control of two different promoters.

Embodiment 389. The nucleic acid molecule, the system, or the cell of embodiment 387, wherein the targeting sequence and the first nucleic acid sequence are under control of a same promoter.

Embodiment 390. The nucleic acid molecule, the system, or the cell of any one of embodiments 387-389, wherein the targeting sequence and the second nucleic acid sequence are under control of two different promoters.

Embodiment 391. The nucleic acid molecule, the system, or the cell of any one of embodiments 387-389, wherein the targeting sequence and the second nucleic acid sequence are under control of a same promoter.

Embodiment 392. The nucleic acid molecule, the system, or the cell of any one of embodiments 387-391, wherein the targeting sequence and the third nucleic acid sequence are under control of two different promoters.

Embodiment 393. The nucleic acid molecule, the system, or the cell of any one of embodiments 387-391, wherein the targeting sequence and the third nucleic acid sequence are under control of a same promoter.

Embodiment 394. The nucleic acid molecule, the system, or the cell of any one of embodiments 387-393, wherein the targeting sequence encodes a chimeric antigen receptor (CAR).

Embodiment 395. The nucleic acid molecule, the system, or the cell of embodiment 394, wherein the chimeric antigen receptor (CAR) comprises a ligand binding domain, and wherein the ligand binding domain targets CD19, CD20, CD22, CD30, CD33, CD38, CD70, CD123, CD138, CD171, CD5, CD7, MUC1, AFP, CEA, PSCA, PSMA, HER2, EGFR, IL13Ralpha2, GD2, NKG2D, EGFTvIII, CS1, CCL1, BCMA, Mesothelin, ROR1, FLT3, FAP, TAG72, CD38, CD44v6, CEA, EPCAM, B7H3, KIT, IL-13Ralpha2, PRSS21, VEGR2, LewisY, CD24, PDGFR-beta, SSEA-4, AFP, NCAM, Claudin18.2, GPC3, GM3, TGS5, HMWMAA, TEM7R, CLDN6, GPRC5D, CXORF61, CD97, CD179a, ALK, PLAC1, GloboH, NY-BR-1, UPK2, HAVCR1, ADRB3, PANX3, GPR20, LY6K, OR51E2, TRAP, WT1, NY-ESO-1, LAGE-1a, or MAGE-A1.

Embodiment 396. The nucleic acid molecule, the system, or the cell of embodiment 395, wherein the ligand binding domain targets CD19.

Embodiment 397. The nucleic acid molecule, the system, or the cell of any one of embodiments 387-396, wherein the targeting sequence encodes CAR19.

Embodiment 398. The nucleic acid molecule, the system, or the cell of any one of embodiments 394-397, wherein the CAR comprises an amino acid sequence that is at least 75%, 80%, 85%, 90%, 95%, 98%, 99%, or 100% identical to any of the sequences set forth in SEQ ID NOs: 4 or 400-407.

Embodiment 399. The nucleic acid molecule, the system, or the cell of any one of embodiments 394-397, wherein the CAR is encoded by a nucleic acid sequence that is at least 75%, 80%, 85%, 90%, 95%, 98%, 99%, or 100% identical to any of the sequences set forth in SEQ ID NOs: 54 or 450-457.

Embodiment 400. The nucleic acid molecule, the system, or the cell of any one of embodiments 196-204, wherein the targeting sequence is linked to the first nucleic acid sequence, the second nucleic acid sequence, or the third nucleic acid sequence via a nucleic acid sequence encoding a cleavable linker.

Embodiment 401. The nucleic acid molecule, the system, or the cell of any one of embodiments 278-280 or 400, wherein the cleavable linker comprises P2A peptide, T2A peptide, E2A peptide, F2A peptide, or IRES peptide.

Embodiment 402. The nucleic acid molecule, the system, or the cell of embodiment 401, wherein the cleavable linker is encoded by a nucleic acid sequence having at least at least 75%, 80%, 85%, 90%, 95%, 98%, 99%, or 100% identical to any of the sequences set forth in SEQ ID NOs: 350-355.

Embodiment 403. The nucleic acid molecule, the system, or the cell of any one of embodiments 278-280 or 400-402, wherein the cleavable linker connects the CAR and the first signal peptide, the second signal peptide and/or the third signal peptide.

Embodiment 404. The nucleic acid molecule, the system, or the cell of any one of embodiments 278-280 or 400-403, wherein the cleavable linker connects the CAR and the first peptide anchor, the second peptide anchor and/or the third peptide anchor.

Embodiment 405. The nucleic acid molecule, the system, or the cell of any one of embodiments 278-280 or 400-404, wherein the cleavable linker connects the CAR and the first non-peptide anchor attachment signal, the second non-peptide anchor attachment signal and/or the third non-peptide anchor attachment signal.

Embodiment 406. The nucleic acid molecule, the system, or the cell of any one of embodiments 278-280 or 400-405, wherein the cleavable linker comprises an amino acid sequence that is at least 75%, 80%, 85%, 90%, 95%, 98%, 99%, or 100% identical to any of the sequences set forth in SEQ ID NOs: 300-303.

Embodiment 407. The nucleic acid molecule of any one of embodiments 256 or 266-406, the system of any one of embodiments 257 or 266-406, or the cell of any one of embodiments 258-406, wherein each of the first polypeptide, the first protein, the second polypeptide, the second protein, the third polypeptide, and the third protein independently further comprises a peptide linker.

Embodiment 408. The nucleic acid molecule, the system, or the cell of embodiment 407, wherein the peptide linker connects the first cytokine peptide to the first peptide anchor, connects the second cytokine peptide to the second peptide anchor and/or connects the third cytokine peptide to the third peptide anchor.

Embodiment 409. The nucleic acid molecule, the system, or the cell of embodiment 406 or 407, wherein the peptide linker connects the first cytokine peptide to the first non-peptide anchor attachment signal, connects the second cytokine peptide to the second non-peptide anchor attachment signal, and/or connects the third cytokine peptide to the third non-peptide anchor attachment signal.

Embodiment 410. The nucleic acid molecule, the system, or the cell of any one of embodiments 406-409, wherein the peptide linker connects the first peptide anchor, the peptide anchor, and/or the third peptide anchor to the cleavable linker.

Embodiment 411. The nucleic acid molecule, the system, or the cell of any one of embodiments 406-410, wherein the peptide linker connects the non-peptide anchor attachment signal to the cleavable linker.

Embodiment 412. The nucleic acid molecule, the system, or the cell of any one of embodiments 406-411, wherein the peptide linker comprises a GS linker, a Lr1 linker, or a Lr8 linker.

Embodiment 413. The nucleic acid molecule, the system, or the cell of any one of embodiments 406-412, wherein the peptide linker comprises an amino acid sequence that is at least 75%, 80%, 85%, 90%, 95%, 98%, 99%, or 100% identical to any of the sequences set forth in SEQ ID NOs: 500, 501, 504, 506, or 507, or the sequence of LE, AS, GSG, or EF.

Embodiment 414. The nucleic acid molecule of any one of embodiments 256 or 266-413, wherein the nucleic acid molecule is RNA.

Embodiment 415. The nucleic acid molecule of any one of embodiments 256 or 266-413, wherein the nucleic acid molecule is DNA.

Embodiment 416. The nucleic acid molecule of any one of embodiments 256 or 266-414, wherein the nucleic acid molecule is a linear RNA.

Embodiment 417. The nucleic acid molecule of any one of embodiments 256 or 266-414, wherein the nucleic acid molecule is a circular RNA.

Embodiment 418. The nucleic acid molecule of any one of embodiments 256 or 266-417, wherein the nucleic acid molecule is a vector.

Embodiment 419. The nucleic acid molecule of embodiment 418, wherein the vector is a viral vector.

Embodiment 420. The nucleic acid molecule of embodiment 418, wherein the vector is a retroviral vector, a lentiviral vector, an adenoviral vector, or an adeno-associated viral vector.

Embodiment 421. The system of any one of embodiments 257 or 266-413, wherein the targeting sequence and the first nucleic acid sequence are present in a same plasmid inside the system.

Embodiment 422. The system of any one of embodiments 257 or 266-413, wherein the targeting sequence and the first nucleic acid sequence are present in two different plasmids inside the system.

Embodiment 423. The system of any one of embodiments 257, 266-413, or 421-422, wherein the targeting sequence and the second nucleic acid sequence are present in a same plasmid inside the system.

Embodiment 424. The system of any one of embodiments 257, 266-413, or 421-422, wherein the targeting sequence and the second nucleic acid sequence are present in two different plasmids inside the system.

Embodiment 425. The system of any one of embodiments 257, 266-413, or 421-424, wherein the targeting sequence and the third nucleic acid sequence are present in a same plasmid inside the system.

Embodiment 426. The system of any one of embodiments 257, 266-413, or 421-424, wherein the targeting sequence and the third nucleic acid sequence are present in two different plasmids inside the system.

Embodiment 427. The system of any one of embodiments 257, 266-413, or 421-426, wherein the first nucleic acid sequence and the second nucleic acid sequence are present in a same plasmid inside the system.

Embodiment 428. The system of any one of embodiments 257, 266-413, or 421-426, wherein the first nucleic acid sequence and the second nucleic acid sequence are present in two different plasmids inside the system.

Embodiment 429. The system of any one of embodiments 257, 266-413, or 421-428, wherein the first nucleic acid sequence and the third nucleic acid sequence are present in a same plasmid inside the system.

Embodiment 430. The system of any one of embodiments 257, 266-413, or 421-428, wherein the first nucleic acid sequence and the third nucleic acid sequence are present in two different plasmids inside the system.

Embodiment 431. The system of any one of embodiments 257, 266-413, or 421-430, wherein the second nucleic acid sequence and the third nucleic acid sequence are present in a same plasmid inside the system.

Embodiment 432. The system of any one of embodiments 257, 266-413, or 421-430, wherein the second nucleic acid sequence and the third nucleic acid sequence are present in two different plasmids inside the system.

Embodiment 433. The cell of any one of embodiments 258-413, wherein the cell is a bacterial cell, yeast cell, or an insect cell.

Embodiment 434. The cell of any one of embodiments 258-413 or 433, wherein the cell is an immune cell or a tumor cell.

Embodiment 435. The cell of any one of embodiments 258-413 or 433-434, wherein the cell is an engineered immune cell.

Embodiment 436. The cell of any one of embodiments 258-413 or 433-434, wherein the cell is a T cell.

Embodiment 437. The cell of any one of embodiments 258-413 or 433-434, wherein the cell is a tumor infiltrating lymphocyte (TIL).

Embodiment 438. The cell of embodiment 435, wherein the engineered immune cell is a natural killer (NK) cell.

Embodiment 439. The cell of any one of embodiments 258, 261-413, or 433-438, wherein the targeting sequence and the first nucleic acid sequence are present in a same plasmid inside the cell.

Embodiment 440. The cell of any one of embodiments 258, 261-413, or 433-438, wherein the targeting sequence and the first nucleic acid sequence are present in two different plasmids inside the cell.

Embodiment 441. The cell of any one of embodiments 258, 263-413, or 433-440, wherein the targeting sequence and the second nucleic acid sequence are present in a same plasmid inside the cell.

Embodiment 442. The cell of any one of embodiments 258, 263-413, or 433-440, wherein the targeting sequence and the second nucleic acid sequence are present in two different plasmids inside the cell.

Embodiment 443. The cell of any one of embodiments 258, 265-413, or 433-442, wherein the targeting sequence and the third nucleic acid sequence are present in a same plasmid inside the cell.

Embodiment 444. The cell of any one of embodiments 258, 265-413, or 433-442, wherein the targeting sequence and the third nucleic acid sequence are present in two different plasmids inside the cell.

Embodiment 445. The cell of any one of embodiments 258, 263-413, or 433-444, wherein the first nucleic acid sequence and the second nucleic acid sequence are present in a same plasmid inside the cell.

Embodiment 446. The cell of any one of embodiments 258, 263-413, or 433-444, wherein the first nucleic acid sequence and the second nucleic acid sequence are present in two different plasmids inside the cell.

Embodiment 447. The cell of any one of embodiments 258, 265-413, or 433-446, wherein the first nucleic acid sequence and the third nucleic acid sequence are present in a same plasmid inside the cell.

Embodiment 448. The cell of any one of embodiments 258, 265-413, or 433-446, wherein the first nucleic acid sequence and the third nucleic acid sequence are present in two different plasmids inside the cell.

Embodiment 449. The cell of any one of embodiments 258, 265-413, or 433-448, wherein the second nucleic acid sequence and the third nucleic acid sequence are present in a same plasmid inside the cell.

Embodiment 450. The cell of any one of embodiments 258, 265-413, or 433-448, wherein the second nucleic acid sequence and the third nucleic acid sequence are present in two different plasmids inside the cell.

Embodiment 451. The cell of any one of embodiments 258, 265-413, or 433-450, wherein the targeting sequence, the first nucleic acid sequence, the second nucleic acid sequence, and the third nucleic acid sequence are present in a genome of the cell.

Embodiment 452. The cell of any one of embodiments 434-451, wherein cytotoxicity of said immune cell is increased relative to a comparable immune cell that
(a) does not comprise all of: (1) the first nucleic acid sequence encodes the first polypeptide comprising the first cytokine peptide and (i) a first non-peptide anchor attachment signal or (ii) the first peptide anchor; (2) the second nucleic acid sequence encodes the second polypeptide comprising the second cytokine peptide and (i) the second non-peptide anchor attachment signal or (ii) the second peptide anchor; and (3) the third nucleic acid sequence encodes the third polypeptide comprising the third cytokine peptide and (i) the third non-peptide anchor attachment signal or (ii) the third peptide anchor; or
(b) does not comprise all of: (1) the first protein comprising the first cytokine peptide and (i) the first non-peptide anchor or (ii) the first peptide anchor; (2) the second protein comprising the second cytokine peptide and (i) the second non-peptide anchor or (ii) the second peptide anchor; and (3) the third protein comprising the third cytokine peptide and (i) the third non-peptide anchor or (ii) the third peptide anchor.

Embodiment 453. The cell of embodiment 452, wherein said increase in cytotoxicity of said immune cell is measured by an in vitro cytotoxicity assay described in Examples A-D.

Embodiment 454. The cell of embodiment 452, wherein said increase in cytotoxicity of said immune cell is measured in vitro or in vivo.

Embodiment 455. The cell of embodiment 452, wherein said increase in cytotoxicity of said immune cell is at least about 5%, 10%, 20%, 30%, 40%, 50% or more.

Embodiment 456. The cell of any one of embodiments 434-455, wherein a population of said immune cells proliferates for a longer period of time relative to a population of comparable immune cells that
(a) does not comprise all of: (1) the first nucleic acid sequence encodes the first polypeptide comprising the first cytokine peptide and (i) a first non-peptide anchor attachment signal or (ii) the first peptide anchor; (2) the second nucleic acid sequence encodes the second polypeptide comprising the second cytokine peptide and (i) the second non-peptide anchor attachment signal or (ii) the second peptide anchor; and (3) the third nucleic acid sequence encodes the third polypeptide comprising the third cytokine peptide and (i) the third non-peptide anchor attachment signal or (ii) the third peptide anchor; or
(b) does not comprise all of: (1) the first protein comprising the first cytokine peptide and (i) the first non-peptide anchor or (ii) the first peptide anchor; (2) the second protein comprising the second cytokine peptide and (i) the second non-peptide anchor or (ii) the second peptide anchor; and (3) the third protein comprising the third cytokine peptide and (i) the third non-peptide anchor or (ii) the third peptide anchor.

Embodiment 457. The cell of embodiment 456, wherein proliferation of said population of said immune cells is measured by an in vitro immune cell proliferation assay described in Examples A-E.

Embodiment 458. The cell of embodiment 456, wherein proliferation of said population of said immune cells is measured in vitro or in vivo.

Embodiment 459. The cell of embodiment 456, wherein proliferation of said population of said immune cells lasts for a period of time at least about 5%, 10%, 20%, 30%, 40%, 50%, 100%, 2 times, 3 times, 5 times, 10 times or longer than said population of said comparable immune cells.

Embodiment 460. A pharmaceutical composition comprising the immune cell of any one of embodiments 2-12, 22-65, or 75-89 or the cell of any one of embodiments 14-65, 73-89, 96-221, 235-255, 258-413, or 433-459, and a pharmaceutically acceptable excipient or carrier.

Embodiment 461. A kit, comprising:
(a) the immune cell of any one of embodiments 2-12, 22-65, or 75-89, the cell of any one of embodiments 14-65, 73-89, 96-221, 235-255, 258-413, or 433-459, or the pharmaceutical composition of embodiment 460); and
(b) an information material containing instructions for administering a dosage of the immune cell, the cell, or a dosage form of the pharmaceutical composition to a subject.

Embodiment 462. A method of treating a subject in need thereof, the method comprising administering to the subject the immune cell of any one of embodiments 2-12, 22-65, or 75-89, the cell of any one of embodiments 14-65, 73-89, 96-221, 235-255, 258-413, or 433-459, or the pharmaceutical composition of embodiment 460.

Embodiment 463. The method of embodiment 462, wherein the immune cell or the cell is allogeneic to the subject.

Embodiment 464. The method of embodiment 462, wherein the immune cell or the cell is autologous to the subject.

Embodiment 465. The method of embodiment 464, further comprising obtaining a population of immune cells, and engineering the population of immune cells or a progeny thereof to produce the engineered immune cell.

Embodiment 466. The method of any one of embodiments 462-465, wherein the immune cell, the cell, or the pharmaceutical composition treats a cancer in the subject.

Embodiment 467. The method of embodiment 466, wherein the cancer comprises a solid tumor.

Embodiment 468. The method of embodiment 466, wherein the cancer comprises leukemia.

Embodiment 469. The method of embodiment 466, wherein the cancer comprises melanoma.

Embodiment 470. The method of embodiment 466, wherein the cancer comprises lymphoma.

Embodiment 471. The method of embodiment 466, wherein the cancer comprises adrenal gland cancer, bladder cancer, bone cancer, brain tumor, breast cancer, cervical cancer, colorectal cancer, esophageal cancer, fallopian tube cancer, gastrointestinal cancer, glioma, glioblastoma, head and neck cancer, hematopoietic malignancy, leukemia, liver cancer, lung cancer, lymphoma, myeloma, nasal cancer, nasopharyngeal cancer, oral cancer, oropharyngeal cancer, ovarian cancer, pancreatic cancer, prostate cancer, sarcoma, stomach cancer, squamous cell lung cancer, testicular cancer, thyroid cancer, uterine cancer, or any combination thereof.

Embodiment 472. The method of any one of embodiments 462-471, wherein the subject has not received lymphodepletion prior to the administration.

Embodiment 473. Use of the immune cell of any one of embodiments 2-12, 22-65, or 75-89, the cell of any one of embodiments 14-65, 73-89, 96-221, 235-255, 258-413, or 433-459, or the pharmaceutical composition of embodiment 460, in the manufacture of a medicament to treat a subject.

Embodiment 474. The use according to embodiment 473, wherein the immune cell or the cell is allogeneic to the subject.

Embodiment 475. The use according to embodiment 473, wherein the immune cell or the cell is autologous to the subject.

Embodiment 476. The use according to any one of embodiments 473-475, wherein the immune cell or the cell treats a cancer in the subject.

Embodiment 477. The use according to embodiment 476, wherein the cancer comprises a solid tumor.

Embodiment 478. The use according to embodiment 476, wherein the cancer comprises leukemia.

Embodiment 479. The use according to embodiment 476, wherein the cancer comprises melanoma.

Embodiment 480. The use according to embodiment 476, wherein the cancer comprises lymphoma.

Embodiment 481. The use according to embodiment 476, wherein the cancer comprises adrenal gland cancer, bladder cancer, bone cancer, brain tumor, breast cancer, cervical cancer, colorectal cancer, esophageal cancer, fallopian tube cancer, gastrointestinal cancer, glioma, glioblastoma, head and neck cancer, hematopoietic malignancy, leukemia, liver cancer, lung cancer, lymphoma, myeloma, nasal cancer, nasopharyngeal cancer, oral cancer, oropharyngeal cancer, ovarian cancer, pancreatic cancer, prostate cancer, sarcoma, stomach cancer, squamous cell lung cancer, testicular cancer, thyroid cancer, uterine cancer, or any combination thereof.

Embodiment 482. A method for making an engineered immune cell, comprising introducing the nucleic acid molecule of any one of embodiments 1, 8-13, 16-72, 90, 91, 94, 104-228, 256 or 266-420, or the system of any one of embodiments 92, 93, 95, 104-221, 229-232, 257, 266-413, or 421-430 into an immune cell.

Embodiment 483. A method for making the immune cell of any one of embodiments 2-12, 22-65, or 75-89 or the cell of any one of embodiments 14-65 or 73-89, 96-221, 235-255, 258-413, or 434-459.

Embodiment 484. A method for making a pharmaceutical composition, the method comprising combining the immune cell of any one of embodiments 2-12, 22-65, or 75-89 or the cell of any one of embodiments 14-65, 73-89, 96-221, 235-255, 258-413, or 434-459, with a pharmaceutically acceptable excipient or carrier.

Embodiment 485. A nucleic acid molecule comprising a nucleic acid sequence that is at least 75%, 80%, 85%, 90%, 95%, 98%, 99%, or 100% identical to any of the sequences set forth in SEQ ID NOs: 510-514, 518-519, 521-522, 527-529, 533, 600-602, 606, 611-617, 625-638, 652-654, 658-666, 676-686, or 698.

Embodiment 486. A polypeptide comprising an amino acid sequence that is at least 75%, 80%, 85%, 90%, 95%, 98%, 99%, or 100% identical to any of the sequences set forth in SEQ ID NOs: 515-517, 523-526, 530-532, 534, 603-605, 607, 618-624, 639-651, 655-657, 667-675, 687-697, or 699.

Embodiment 487. A nucleic acid molecule comprising a nucleic acid sequence that encodes the polypeptide of embodiment 486.

Embodiment 488. The immune cell of any one of embodiments 3, 7-12, 22-61, or 71-84, wherein the immune cell does not comprise a stimulus response element (SRE) and wherein the cytokine peptide is IL-12p40.

1.-102. (canceled)

103. A nucleic acid molecule comprising a first nucleic acid sequence and a second nucleic acid sequence, wherein:
(a) the first nucleic acid sequence encodes a first polypeptide comprising a first cytokine peptide and (i) a first non-peptide anchor attachment signal or (ii) a first peptide anchor;
(b) the second nucleic acid sequence encodes a second polypeptide comprising a second cytokine peptide and (i) a second non-peptide anchor attachment signal or (ii) a second peptide anchor; and
wherein the first cytokine peptide is a proinflammatory cytokine peptide, and the second cytokine peptide is an anti-inflammatory cytokine peptide.

104. The nucleic acid molecule of claim 103, wherein the wherein the anti-inflammatory cytokine peptide comprises at least a portion of IL-10, IL-4, or IL-27, or a variant thereof.

105. The nucleic acid molecule of claim 103, wherein the proinflammatory cytokine peptide comprises at least a portion of IL-2, IL-7, IL-9, IL-12, IL-12p40, IL-15, IL-18, IL-21, IL-23, IL-36γ, IL-23p19, or IL-1α, or a variant thereof.

106. The nucleic acid molecule of claim 103, wherein the anti-inflammatory cytokine peptide is encoded by a nucleic acid sequence that is at least 75%, 80%, 85%, 90%, 95%, 98%, 99%, or 100% identical to the sequence set forth in SEQ ID NO: 154, 151, 163, 164, or 159.

107. The nucleic acid molecule of claim 103, wherein the proinflammatory cytokine peptide is encoded by a nucleic acid sequence that is at least 75%, 80%, 85%, 90%, 95%, 98%, 99%, or 100% identical to any one of the sequences set forth in SEQ ID NOs: 150, 152, 153, 155-158, or 160-162.

108. The nucleic acid molecule of claim 103, wherein the second nucleic acid sequence and the first nucleic acid sequence are linked via a nucleic acid sequence encoding a cleavable linker, wherein the cleavable linker comprises P2A peptide, T2A peptide, E2A peptide, F2A peptide, or IRES peptide.

109. The nucleic acid molecule of claim 108, wherein the cleavable linker comprises an amino acid sequence that is at least 75%, 80%, 85%, 90%, 95%, 98%, 99%, or 100% identical to any of the sequences set forth in SEQ ID NOs: 300-303.

110. The nucleic acid molecule of claim 103, wherein
(a) the first polypeptide comprises a first signal peptide, wherein the first signal peptide, the first cytokine peptide, and (i) the first non-peptide anchor attachment signal or (ii) the first peptide anchor are operably linked in a direction from a N-terminal of the polypeptide to a C-terminal of the polypeptide; and/or
(b) the second polypeptide comprises a second signal peptide, wherein the second signal peptide, the second cytokine peptide, and (i) the second non-peptide anchor attachment signal or (ii) the second peptide anchor are operably linked in a direction from a N-terminal of the polypeptide to a C-terminal of the polypeptide.

111. The nucleic acid molecule of claim 110, wherein the first signal peptide or the second signal peptide comprises an amino acid sequence that is at least 75%, 80%, 85%, 90%, 95%, 98%, 99%, or 100% identical to the sequence set forth in SEQ ID NO: 114 or 113.

112. The nucleic acid molecule of claim 103, wherein the nucleic acid molecule further comprises a targeting sequence encoding a targeting moiety, and wherein the targeting moiety comprises a chimeric antigen receptor (CAR), a T cell receptor, a B cell receptor, or any combination thereof.

113. The nucleic acid molecule of claim 112, wherein the CAR comprises an amino acid sequence that is at least 75%, 80%, 85%, 90%, 95%, 98%, 99%, or 100% identical to any of the sequences set forth in SEQ ID NOs: 4 or 400-407.

114. The nucleic acid molecule of claim 103, wherein (a) the first non-peptide anchor attachment signal comprises a glycolipid-attachment signal, and/or (b) the second non-peptide anchor attachment signal comprises a glycolipid-attachment signal; wherein the glycolipid-attachment signal comprises a GPI-attachment signal.

115. The nucleic acid molecule of claim 114, wherein the GPI-attachment signal comprises an amino acid sequence that is at least 75%, 80%, 85%, 90%, 95%, 98%, 99%, or 100% identical to the sequence set forth in SEQ ID NO: 200.

116. The nucleic acid molecule of claim 103, wherein the first polypeptide or the second polypeptide independently further comprises a peptide linker.

117. The nucleic acid molecule of claim 116, wherein the peptide linker comprises an amino acid sequence that is at least 75%, 80%, 85%, 90%, 95%, 98%, 99%, or 100% identical to any of the sequences set forth in SEQ ID NOs: 506, 500, 501, 504, or 507, or the sequence of LE, AS, GSG, or EF.

118. The nucleic acid molecule of claim 103, wherein the first nucleic acid sequence or the second nucleic acid sequence comprises a nucleic acid sequence that is at least 75%, 80%, 85%, 90%, 95%, 98%, 99%, or 100% identical to any of the sequences set forth in SEQ ID NOs: 512, 521, 522, 510, 511, 513-514, 518-519, 527-529, 533, 600-602, 606, 611-617, 625-638, 652-654, 658-666, 676-686, or 698.

119. The nucleic acid molecule of claim 103, wherein the first polypeptide or the second polypeptide comprises an amino acid sequence that is at least 75%, 80%, 85%, 90%, 95%, 98%, 99%, or 100% identical to any of the sequences set forth in SEQ ID NOs: 516, 525, 526, 515, 517, 523, 524, 526, 530-532, 534, 603-605, 607, 618-624, 639-651, 655-657, 667-675, 687-697, or 699.

120. A nucleic acid molecule comprising a nucleic acid sequence that is at least 75%, 80%, 85%, 90%, 95%, 98%, 99%, or 100% identical to any of the sequences set forth in SEQ ID NOs: 512, 521, 522, 510, 511, 513-514, 518-519, 527-529, 533, 600-602, 606, 611-617, 625-638, 652-654, 658-666, 676-686, or 698.

121. A polypeptide comprising an amino acid sequence that is at least 75%, 80%, 85%, 90%, 95%, 98%, 99%, or 100% identical to any of the sequences set forth in SEQ ID NOs: 516, 525, 526, 515, 517, 523, 524, 526, 530-532, 534, 603-605, 607, 618-624, 639-651, 655-657, 667-675, 687-697, or 699.

122. A cell comprising the nucleic acid molecule of claim 103.

123. A method of making an engineered immune cell, comprising introducing the nucleic acid molecule of claim 103 into an immune cell.

124. A method of treating a disease in a subject in need thereof, comprising administering to the subject a therapeutically effective amount of the cell of claim 122.


SEQUENCE LISTING (711 sequences)

SEQ ID NO: 1 - skipped

SEQ ID NO: 2 - protein, 23 aa, synthetic construct
AVMAPRTLLLLLSGALALTQTWA

SEQ ID NO: 3 - protein, 20 aa, synthetic construct
GTSLLCWMALCLLGADHADA

SEQ ID NO: 4 - protein, 19 aa, synthetic construct
SRSVALAVLALLSLSGLEA

SEQ ID NO: 5 - protein, 17 aa, synthetic construct
LLAMVLTSALLLCSVAG

SEQ ID NO: 6 - protein, 35 aa, synthetic construct
AAEPVEDNCINFVAMKFIDNTLYFIAEDDENLESD

SEQ ID NO: 7 - protein, 16 aa, synthetic construct
RGTPGDADGGGRAVYQ

SEQ ID NO: 8-51 - skipped (44 entries)

SEQ ID NO: 52 - DNA, 69 bp, synthetic construct
  source 1..69: mol_type = other DNA
gctgtgatggcccctagaaccctgctgctgctgctgagcggcgccctggccctgacacagacctgggcc

SEQ ID NO: 53 - DNA, 60 bp, synthetic construct
  source 1..60: mol_type = other DNA
ggcaccagcctgctgtgctggatggcactgtgcctgctgggagcagaccacgccgatgcc

SEQ ID NO: 54 - DNA, 57 bp, synthetic construct
  source 1..57: mol_type = other DNA
tctcgctccgtggccttagctgtgctcgcgctactctctctttctggcctggaggct

SEQ ID NO: 55 - DNA, 51 bp, synthetic construct
  source 1..51: mol_type = other DNA
ctgctggccatggtgctgacaagcgccctgctgctgtgctctgtggccggg

SEQ ID NO: 56 - DNA, 105 bp, synthetic construct
  source 1..105: mol_type = other DNA
gccgctgagcccgtggaggacaactgcatcaacttcgtggccatgaagttcatcgacaacaccctgtactttatcgccgaggacgacgagaacctggagagcgac

SEQ ID NO: 57 - DNA, 48 bp, synthetic construct
  source 1..48: mol_type = other DNA
agagggacccccggggacgccgacggaggaggaagagcagtgtaccag

SEQ ID NO: 58 - DNA, 69 bp, synthetic construct
  source 1..69: mol_type = other DNA
ggtctcacctcccaactgcttccccctctgttcttcctgctagcatgtgccggcaactttgtccacgga

SEQ ID NO: 59 - DNA, 51 bp, synthetic construct
  source 1..51: mol_type = other DNA
cacagctcagcactgctctgttgcctggtcctcctgactggggtgagggcc

SEQ ID NO: 60-99 - skipped (40 entries)

SEQ ID NO: 100 - protein, 133 aa, synthetic construct [sequence omitted]

SEQ ID NO: 101 - protein, 129 aa, synthetic construct [sequence omitted]

SEQ ID NO: 102 - protein, 152 aa, synthetic construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 103 - protein, 126 aa, synthetic construct
QGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKI

SEQ ID NO: 104 - protein, 160 aa, synthetic construct
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 105 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 106 - protein, 135 aa, synthetic construct
GIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRA

SEQ ID NO: 107 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 108 - protein, 135 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRA

SEQ ID NO: 109 - protein, 215 aa, synthetic construct
FPRPPGRPQLSLQELRREFTVSLHLARKLLSEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 110 - protein, 152 aa, synthetic construct
SMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNIND

SEQ ID NO: 111 - protein, 170 aa, synthetic construct
RAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 112 - protein, 271 aa, synthetic construct
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 113 - protein, 23 aa, synthetic construct
GLTSQLLPPLFFLLACAGNFVHG

SEQ ID NO: 114 - protein, 17 aa, synthetic construct
HSSALLCCLVLLTGVRA

SEQ ID NO: 115-149 - skipped (35 entries)

SEQ ID NO: 150 - DNA, 399 bp, synthetic construct
  source 1..399: mol_type = other DNA
gcccctaccagcagctccaccaagaagacccagctgcagctggagcacctcctgctggacctgcagatgatcctgaacggcatcaacaactataagaatcctaagctgacaagaatgctgacctttaagttctacatgcctaagaaagctaccgagctgaagcacctgcaatgtctggaagaggagctgaaacctctggaagaggtgctgaatctggcccagagcaaaaacttccacctcagacctagagatctgatcagcaacatcaacgtgatcgtgctggaactgaagggcagcgagacaacattcatgtgcgagtacgccgacgagacagccaccattgtggaattcctgaaccggtggatcaccttctgccagtccatcatctctacactgacc

SEQ ID NO: 151 - DNA, 387 bp, synthetic construct
  source 1..387: mol_type = other DNA
cacaagtgcgacatcaccctgcaagagatcatcaagaccctgaacagcctgaccgagcagaagaccctgtgcaccgagctgaccgtgaccgacatcttcgccgctagcaagaacaccaccgagaaggagaccttctgcagagccgccaccgtgctgagacagttctacagccaccacgagaaggacacaagatgcctgggcgccaccgctcagcagttccacagacacaagcagctgatcagattcctgaagagactggacagaaacctgtggggcctggccggcctgaacagctgccccgtgaaggaggccaatcagagcaccctggagaacttcctggagagactgaagaccatcatgagagagaagtacagcaagtgcagcagc

SEQ ID NO: 152 - DNA, 456 bp, synthetic construct
  source 1..456: mol_type = other DNA
gattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacac

SEQ ID NO: 153 - DNA, 378 bp, synthetic construct
  source 1..378: mol_type = other DNA
caggggtgccctaccctggctggaatcctggacatcaacttcctgatcaacaagatgcaggaggaccccgcttccaagtgccactgctccgccaatgtgacctcctgcctgtgtctgggcattccctccgacaactgtactagaccctgcttcagcgagcgcctgagccagatgactaacaccaccatgcagacccgctaccccctgatcttctcccgcgtcaagaagtccgtcgaagtgctgaagaacaacaagtgcccctacttctcctgcgagcagccctgcaaccagaccaccgccggcaacgccctgaccttcctgaaaagcctgctggagatcttccagaaggagaaaatgcgcggcatgagaggcaagatc

SEQ ID NO: 154 - DNA, 480 bp, synthetic construct
  source 1..480: mol_type = other DNA
agccccgggcaaggcacacagagcgagaacagctgcacccacttccccggcaacctgcccaacatgctgagagacctgagagacgccttcagcagagtgaagaccttctttcagatgaaggatcagctggacaacctgctcctgaaggagagcctgctggaggacttcaagggctacctgggctgccaagccctgagcgagatgattcagttctacctggaagaggtgatgccccaagccgagaaccaagaccccgacatcaaggcccacgtgaacagcctgggcgagaacctgaagaccctgagactgagactgagaagatgccacagattcctgccctgcgagaacaagagcaaggccgtggagcaagtgaagaacgccttcaacaagctgcaagagaagggcatctacaaggccatgagcgagttcgacatcttcatcaactacatcgaggcctacatgaccatgaagatcagaaac

SEQ ID NO: 155 - DNA, 918 bp, synthetic construct
  source 1..918: mol_type = other DNA
atatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagt

SEQ ID NO: 156 - DNA, 405 bp, synthetic construct
  source 1..405: mol_type = other DNA
ggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagcc

SEQ ID NO: 157 - DNA, 471 bp, synthetic construct
  source 1..471: mol_type = other DNA
tactttggcaagctggagagcaagctgagcgtgatccggaacctgaacgaccaggtgctgttcatcgaccagggcaatcggcctctgtttgaggacatgaccgacagcgactgcagagacaacgcacccagaaccatcttcatcatctccatgtacaaggactcccagccaaggggcatggccgtgaccatcagcgtgaaatgcgagaaaatcagcacactgtcatgcgagaacaagatcatcagcttcaaggaaatgaacccccccgacaacatcaaggacacaaaaagcgacatcatcttcttccagagatccgtccccggccacgacaacaaaatgcagttcgagagctcctcctacgagggctacttcctggcctgcgaaaaagaaagagacctgttcaagctgatcctgaagaaggaggacgagctgggcgacagaagcatcatgttcaccgtgcagaacgaggac

SEQ ID NO: 158 - DNA, 405 bp, synthetic construct
  source 1..405: mol_type = other DNA
cagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagct

SEQ ID NO: 159 - DNA, 645 bp, synthetic construct
  source 1..645: mol_type = other DNA
ttccctcggccccccggcagacctcagctgagcctgcaagagctgagaagagagttcaccgtgagcctgcacctggctagaaagctgctgagcgaggtgagaggccaagcccacagattcgccgagagccacctgcccggcgtgaacctgtacctgctgcccctgggcgagcagctgcccgacgtgagcctgaccttccaagcctggagaagactgagcgaccccgagagactgtgcttcatcagcaccaccctgcagcccttccacgccctgctgggcggcctgggcacccaaggcagatggaccaacatggagagaatgcagctgtgggccatgagactggacctgagagacctgcagagacacctgagattccaagtgctggccgccggcttcaacctgcccgaggaagaggaagaggaagaggaggaagaggaggaagagcggaagggcctgctgcctggcgctctcggcagcgccctgcaagggcctgctcaagtgagctggcctcagctgctgagcacctacagactgctgcacagcctggagctggtgctgagcagagccgtgagagagctgctcctgctgagcaaggccggccacagcgtgtggcccctgggcttccccaccctgagccctcagccc

SEQ ID NO: 160 - DNA, 456 bp, synthetic construct
  source 1..456: mol_type = other DNA
agcatgtgcaagcccatcaccgggaccatcaacgacctgaaccagcaggtgtggaccctgcagggacagaacctggtggccgtgcccagaagcgatagcgtgacacccgtgaccgtcgccgtcattacctgcaagtaccccgaagccctggaacagggcagaggcgatcccatctacctggggattcaaaaccccgaaatgtgcctgtactgcgagaaggtcggcgagcagcctaccctgcagctgaaggaacagaaaatcatggacctgtacggacagcctgagcccgtgaaacccttcctgttctacagagctaagaccggccgcaccagcacactggagagcgtcgccttccccgactggttcatcgccagcagcaaaagagaccagcccatcatcctgacctcagagctgggaaagagctacaacaccgccttcgagctgaacatcaacgat

SEQ ID NO: 161 - DNA, 510 bp, synthetic construct
  source 1..510: mol_type = other DNA
agggctgtccccgggggttcctccccggcctggacccagtgtcagcagctttcacagaagctgtgcactctggcctggtctgctcaccccctggtgggccatatggatctgcgcgaggagggcgacgaggaaacgaccaacgacgtgccacacatccagtgcggggacggctgtgatcctcagggtctccgtgacaactcccagttttgcctgcagcgcatccaccagggcctcatcttttacgagaaactgctgggctccgacatcttcaccggcgagccctctctgctgcccgacagcccggttggacagctgcacgcctccctactgggtttgagccagctgctccagccagagggccaccactgggagacccagcaaattccctctcttagtccttcgcagccgtggcaacgcctgcttctccgcttcaagattctgcggtcgttgcaggccttcgtggccgtggcggctcgagtgttcgcgcatggagcagccacactgagccct

SEQ ID NO: 162 - DNA, 813 bp, synthetic construct
  source 1..813: mol_type = other DNA
atggccaaggtgcccgacatgttcgaggacctgaagaactgctacagcgagaacgaggaggacagcagcagcatcgaccacctgagcctgaatcagaagagcttctaccacgtgagctacggccccctgcacgagggctgcatggatcagagcgtgagcctgagcatcagcgagacaagcaagacaagcaagctgaccttcaaggagagcatggtggtcgtggccaccaacggcaaggtgctgaagaagagaagactgagcctgagccaatccattacagacgatgacctggaggccatcgccaacgacagcgaggaagagatcatcaagcctagaagcgcccccttcagcttcctgagcaacgtgaagtacaacttcatgagaatcatcaagtacgagttcatcctgaacgacgccctgaatcagagcatcatcagagccaacgatcagtacctgaccgccgctgccctgcacaacctggacgaggccgtgaagttcgacatgggcgcctacaagagcagcaaggacgacgccaagatcaccgtgatcctgagaatcagcaagacacagctgtacgtgaccgcccaagacgaggatcagcccgtgctgctgaaggagatgcccgagatccccaagaccatcaccggcagcgagaccaacctgctgttcttctgggagacccacggcaccaagaactacttcacaagcgtggcccaccccaacctgttcatcgccaccaagcaagactactgggtgtgcctggccggcggcccccctagcatcaccgactttcagatcctggagaaccaagcc

SEQ ID NO: 163 - DNA, 387 bp, synthetic construct
  source 1..387: mol_type = other DNA
cacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagc

SEQ ID NO: 164 - DNA, 480 bp, synthetic construct
  source 1..480: mol_type = other DNA
agcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaac

SEQ ID NO: 165-199 - skipped (35 entries)

SEQ ID NO: 200 - protein, 29 aa, synthetic construct
LENGGTSLSEKTVLLLVTPFLAAAWSLHP

SEQ ID NO: 201-202 - skipped (2 entries)

SEQ ID NO: 203 - protein, 21 aa, synthetic construct
IYIWAPLAGTCGVLLLSLVIT

SEQ ID NO: 204 - protein, 46 aa, synthetic construct
LLPSWAITLISVNGIFVICCLTYCFAPRCRERRRNERLRRESVRPV

SEQ ID NO: 205-249 - skipped (45 entries)

SEQ ID NO: 250 - DNA, 87 bp, synthetic construct
  source 1..87: mol_type = other DNA
cttgaaaatggtgggacatccttatcagagaaaacagttcttctgctggtgactccatttctggcagcagcctggagccttcatccc

SEQ ID NO: 251 - DNA, 87 bp, synthetic construct
  source 1..87: mol_type = other DNA
ctggagaacggagggacaagcctgagcgagaagacagtgctgctgctggtgaccccatttctggccgcagcatggagcctgcacccc

SEQ ID NO: 252 - DNA, 87 bp, synthetic construct
  source 1..87: mol_type = other DNA
ctggagaacggggggacaagcctgtccgagaagaccgtgctgctgctggtcactcccttcctggccgccgcctggagcctgcatccc

SEQ ID NO: 253 - DNA, 63 bp, synthetic construct
  source 1..63: mol_type = other DNA
atctacatctgggctcctctggctggcacctgcggagtgctgctgctgtctctggtgattact

SEQ ID NO: 254 - DNA, 138 bp, synthetic construct
  source 1..138: mol_type = other DNA
ctgctgcctagttgggccatcaccctgatcagcgtgaacggaatcttcgtgatctgttgtctgacatactgttttgctcccaggtgcagagagaggagaaggaacgagcggctgagaagagaatccgtgcggcctgtg

SEQ ID NO: 255 - DNA, 138 bp, synthetic construct
  source 1..138: mol_type = other DNA
ctgctgccttcttgggctatcaccctgatcagcgtgaacggaatctttgtgatctgttgtctgacttattgtttcgcccccagatgcagagagaggagaaggaatgagagactgagaagagaatcagtgaggcccgtg

SEQ ID NO: 256-299 - skipped (44 entries)

SEQ ID NO: 300 - protein, 19 aa, synthetic construct
ATNFSLLKQAGDVEENPGP

SEQ ID NO: 301 - protein, 18 aa, synthetic construct
EGRGSLLTCGDVEENPGP

SEQ ID NO: 302 - protein, 20 aa, synthetic construct
QCTNYALLKLAGDVESNPGP

SEQ ID NO: 303 - protein, 27 aa, synthetic construct
RAKRSGSGATNFSLLKQAGDVEENPGP

SEQ ID NO: 304-349 - skipped (46 entries)

SEQ ID NO: 350 - DNA, 57 bp, synthetic construct
  source 1..57: mol_type = other DNA
gccacgaacttctctctgttaaagcaagcaggagacgtggaagaaaaccccggtccc

SEQ ID NO: 351 - DNA, 54 bp, synthetic construct
  source 1..54: mol_type = other DNA
gagggcaggggaagtctactaacatgcggggacgtggaggaaaatcccggcccc

SEQ ID NO: 352 - DNA, 60 bp, synthetic construct
  source 1..60: mol_type = other DNA
cagtgtaccaattatgctctgctgaagctggctggggacgtggaaagcaaccccggacct

SEQ ID NO: 353 - DNA, 81 bp, synthetic construct
  source 1..81: mol_type = other DNA
agggccaagaggagcggaagcggggctacaaacttcagtctgctgaagcaggctggcgacgtggaggaaaaccctggacct

SEQ ID NO: 354 - DNA, 574 bp, synthetic construct
  source 1..574: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 355 - DNA, 57 bp, synthetic construct
  source 1..57: mol_type = other DNA
gccacgaacttctctctgttaaagcaagcaggagatgttgaagaaaaccccgggcct

SEQ ID NO: 356-399 - skipped (44 entries)

SEQ ID NO: 400 - protein, 20 aa, synthetic construct
ALPVTALLLPLALLLHAARP

SEQ ID NO: 401 - protein, 242 aa, synthetic construct
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 402 - protein, 45 aa, synthetic construct
TTTPAPRPPTPAPTIASQPLSLRPEACRPAAGGAVHTRGLDFACD

SEQ ID NO: 403 - protein, 24 aa, synthetic construct
IYIWAPLAGTCGVLLLSLVITLYC

SEQ ID NO: 404 - protein, 42 aa, synthetic construct
KRGRKKLLYIFKQPFMRPVQTTQEEDGCSCRFPEEEEGGCEL

SEQ ID NO: 405 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 406 - protein, 485 aa, synthetic construct
ALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 407 - protein, 484 aa, synthetic construct
SRSVALAVLALLSLSGLEADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 408-449 - skipped (42 entries)

SEQ ID NO: 450 - DNA, 60 bp, synthetic construct
  source 1..60: mol_type = other DNA
gccttaccagtgaccgccttgctcctgccgctggccttgctgctccacgccgccaggccg

SEQ ID NO: 451 - DNA, 726 bp, synthetic construct
  source 1..726: mol_type = other DNA
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctca

SEQ ID NO: 452 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 453 - DNA, 72 bp, synthetic construct
  source 1..72: mol_type = other DNA
atctacatctgggcgcccttggccgggacttgtggggtccttctcctgtcactggttatcaccctttactgc

SEQ ID NO: 454 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 455 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 456 - DNA, 1455 bp, synthetic construct
  source 1..1455: mol_type = other DNA
gccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 457 - DNA, 1452 bp, synthetic construct
  source 1..1452: mol_type = other DNA
tctcgctccgtggccttagctgtgctcgcgctactctctctttctggcctggaggctgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 458-499 - skipped (42 entries)

SEQ ID NO: 500 - protein, 4 aa, synthetic construct
RAEF

SEQ ID NO: 501 - protein, 7 aa, synthetic construct
AGTRGSG

SEQ ID NO: 502-503 - skipped (2 entries)

SEQ ID NO: 504 - protein, 7 aa, synthetic construct
AGIDGSG

SEQ ID NO: 505 - skipped

SEQ ID NO: 506 - protein, 27 aa, synthetic construct
SGGGGSGGGGSGGGGSGGGGSGGGSLQ

SEQ ID NO: 507 - protein, 48 aa, synthetic construct
SGGGGSGGGGSGGGGSGGGGSGGGGSGGGGSGGGGSGGGGSGGGGSLQ

SEQ ID NO: 508 - skipped

SEQ ID NO: 509 - DNA, 81 bp, synthetic construct
  source 1..81: mol_type = other DNA
tctggaggaggaggatctggcggaggaggaagtggaggaggaggctctggaggaggcggatctggaggagggagtctgcag

SEQ ID NO: 510 - DNA, 2142 bp, synthetic construct
  source 1..2142: mol_type = other DNA
gccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctccggcgagggcaggggaagtctactaacatgcggggacgtggaggaaaatcccggccccggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctctggaggaggaggatctggcggaggaggaagtggaggaggaggctctggaggaggcggatctggaggagggagtctgcagcttgaaaatggtgggacatccttatcagagaaaacagttcttctgctggtgactccatttctggcagcagcctggagccttcatccc

SEQ ID NO: 511 - DNA, 624 bp, synthetic construct
  source 1..624: mol_type = other DNA
ggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctctggaggaggaggatctggcggaggaggaagtggaggaggaggctctggaggaggcggatctggaggagggagtctgcagcttgaaaatggtgggacatccttatcagagaaaacagttcttctgctggtgactccatttctggcagcagcctggagccttcatccc

SEQ ID NO: 512 - DNA, 2217 bp, synthetic construct
  source 1..2217: mol_type = other DNA
cacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactctggaggaggaggatctggcggaggaggaagtggaggaggaggctctggaggaggcggatctggaggagggagtctgcagcttgaaaatggtgggacatccttatcagagaaaacagttcttctgctggtgactccatttctggcagcagcctggagccttcatcccggctccggcgagggcaggggaagtctactaacatgcggggacgtggaggaaaatcccggccccgccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 513 - DNA, 708 bp, synthetic construct
  source 1..708: mol_type = other DNA
cacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactctggaggaggaggatctggcggaggaggaagtggaggaggaggctctggaggaggcggatctggaggagggagtctgcagcttgaaaatggtgggacatccttatcagagaaaacagttcttctgctggtgactccatttctggcagcagcctggagccttcatcccggctccggc

SEQ ID NO: 514 - DNA, 2049 bp, synthetic construct
  source 1..2049: mol_type = other DNA
gccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctccggcgagggcaggggaagtctactaacatgcggggacgtggaggaaaatcccggcccccacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaac

SEQ ID NO: 515 - protein, 714 aa, synthetic construct
ALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPGLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSSGGGGSGGGGSGGGGSGGGGSGGGSLQLENGGTSLSEKTVLLLVTPFLAAAWSLHP

SEQ ID NO: 516 - protein, 739 aa, synthetic construct
HSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSGGGGSGGGGSGGGGSGGGGSGGGSLQLENGGTSLSEKTVLLLVTPFLAAAWSLHPGSGEGRGSLLTCGDVEENPGPALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 517 - protein, 683 aa, synthetic construct
ALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 518 - DNA, 2118 bp, synthetic construct
  source 1..2118: mol_type = other DNA
gccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctccggcgagggcaggggaagtctactaacatgcggggacgtggaggaaaatcccggccccggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctctggaggaggaggatctggcggaggaggaagtggaggaggaggctctggaggaggcggatctggaggagggagtctgcagatctacatctgggctcctctggctggcacctgcggagtgctgctgctgtctctggtgattact

SEQ ID NO: 519 - DNA, 2193 bp, synthetic construct
  source 1..2193: mol_type = other DNA
gccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctccggcgagggcaggggaagtctactaacatgcggggacgtggaggaaaatcccggccccggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagcagtggcgggggaggatctggaggcggaggatctgggggaggaggaagcggaggaggagggagcggaggaggcagcctgcagctgctgccttcttgggctatcaccctgatcagcgtgaacggaatctttgtgatctgttgtctgacttattgtttcgcccccagatgcagagagaggagaaggaatgagagactgagaagagaatcagtgaggcccgtg

SEQ ID NO: 520 - DNA, 81 bp, synthetic construct
  source 1..81: mol_type = other DNA
agtggcgggggaggatctggaggcggaggatctgggggaggaggaagcggaggaggagggagcggaggaggcagcctgcag

SEQ ID NO: 521 - DNA, 2193 bp, synthetic construct
  source 1..2193: mol_type = other DNA
cacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactctggaggaggaggatctggcggaggaggaagtggaggaggaggctctggaggaggcggatctggaggagggagtctgcagatctacatctgggctcctctggctggcacctgcggagtgctgctgctgtctctggtgattactggctccggcgagggcaggggaagtctactaacatgcggggacgtggaggaaaatcccggccccgccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 522 - DNA, 2193 bp, synthetic construct
  source 1..2193: mol_type = other DNA
gccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctccggcgagggcaggggaagtctactaacatgcggggacgtggaggaaaatcccggcccccacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactctggaggaggaggatctggcggaggaggaagtggaggaggaggctctggaggaggcggatctggaggagggagtctgcagatctacatctgggctcctctggctggcacctgcggagtgctgctgctgtctctggtgattact

SEQ ID NO: 523 - protein, 706 aa, synthetic construct
ALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPGLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSSGGGGSGGGGSGGGGSGGGGSGGGSLQIYIWAPLAGTCGVLLLSLVIT

SEQ ID NO: 524 - protein, 731 aa, synthetic construct
ALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPGLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSSGGGGSGGGGSGGGGSGGGGSGGGSLQLLPSWAITLISVNGIFVICCLTYCFAPRCRERRRNERLRRESVRPV

SEQ ID NO: 525 - protein, 731 aa, synthetic construct
HSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSGGGGSGGGGSGGGGSGGGGSGGGSLQIYIWAPLAGTCGVLLLSLVITGSGEGRGSLLTCGDVEENPGPALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 526 - protein, 731 aa, synthetic construct
ALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRNSGGGGSGGGGSGGGGSGGGGSGGGSLQIYIWAPLAGTCGVLLLSLVIT

SEQ ID NO: 527 - DNA, 2841 bp, synthetic construct
  source 1..2841: mol_type = other DNA
gccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctccggcgagggcaggggaagtctactaacatgcggggacgtggaggaaaatcccggccccggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctctggaggaggaggatctggcggaggaggaagtggaggaggaggctctggaggaggcggatctggaggagggagtctgcagcttgaaaatggtgggacatccttatcagagaaaacagttcttctgctggtgactccatttctggcagcagcctggagccttcatcccggaagcggagccacgaacttctctctgttaaagcaagcaggagatgttgaagaaaaccccgggcctggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagcctctggaggaggaggatctggcggaggaggaagtggaggaggaggctctggaggaggcggatctggaggagggagtctgcagcttgaaaatggtgggacatccttatcagagaaaacagttcttctgctggtgactccatttctggcagcagcctggagccttcatccc

SEQ ID NO: 528 - DNA, 2793 bp, synthetic construct
  source 1..2793: mol_type = other DNA
gccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctccggcgagggcaggggaagtctactaacatgcggggacgtggaggaaaatcccggccccggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctctggaggaggaggatctggcggaggaggaagtggaggaggaggctctggaggaggcggatctggaggagggagtctgcagatctacatctgggctcctctggctggcacctgcggagtgctgctgctgtctctggtgattactggaagcggagccacgaacttctctctgttaaagcaagcaggagatgttgaagaaaaccccgggcctggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagcctctggaggaggaggatctggcggaggaggaagtggaggaggaggctctggaggaggcggatctggaggagggagtctgcagatctacatctgggctcctctggctggcacctgcggagtgctgctgctgtctctggtgattact

SEQ ID NO: 529 - DNA, 1320 bp, synthetic construct
  source 1..1320: mol_type = other DNA
ggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctctggaggaggaggatctggcggaggaggaagtggaggaggaggctctggaggaggcggatctggaggagggagtctgcagcttgaaaatggtgggacatccttatcagagaaaacagttcttctgctggtgactccatttctggcagcagcctggagccttcatcccggctccggcgagggcaggggaagtctactaacatgcggggacgtggaggaaaatcccggccccggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagcctctggaggaggaggatctggcggaggaggaagtggaggaggaggctctggaggaggcggatctggaggagggagtctgcagcttgaaaatggtgggacatccttatcagagaaaacagttcttctgctggtgactccatttctggcagcagcctggagccttcatccc

SEQ ID NO: 530 - protein, 947 aa, synthetic construct
ALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPGLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSSGGGGSGGGGSGGGGSGGGGSGGGSLQLENGGTSLSEKTVLLLVTPFLAAAWSLHPGSGATNFSLLKQAGDVEENPGPGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRASGGGGSGGGGSGGGGSGGGGSGGGSLQLENGGTSLSEKTVLLLVTPFLAAAWSLHP

SEQ ID NO: 531 - protein, 931 aa, synthetic construct
ALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPGLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSSGGGGSGGGGSGGGGSGGGGSGGGSLQIYIWAPLAGTCGVLLLSLVITGSGATNFSLLKQAGDVEENPGPGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRASGGGGSGGGGSGGGGSGGGGSGGGSLQIYIWAPLAGTCGVLLLSLVIT

SEQ ID NO: 532 - protein, 440 aa, synthetic construct
GLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSSGGGGSGGGGSGGGGSGGGGSGGGSLQLENGGTSLSEKTVLLLVTPFLAAAWSLHPGSGEGRGSLLTCGDVEENPGPGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRASGGGGSGGGGSGGGGSGGGGSGGGSLQLENGGTSLSEKTVLLLVTPFLAAAWSLHP

SEQ ID NO: 533 - DNA, 1272 bp, synthetic construct
  source 1..1272: mol_type = other DNA
ggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctctggaggaggaggatctggcggaggaggaagtggaggaggaggctctggaggaggcggatctggaggagggagtctgcagatctacatctgggctcctctggctggcacctgcggagtgctgctgctgtctctggtgattactggctccggcgagggcaggggaagtctactaacatgcggggacgtggaggaaaatcccggccccggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagcctctggaggaggaggatctggcggaggaggaagtggaggaggaggctctggaggaggcggatctggaggagggagtctgcagatctacatctgggctcctctggctggcacctgcggagtgctgctgctgtctctggtgattact

SEQ ID NO: 534 - protein, 424 aa, synthetic construct
GLTSQLLPPLFFLLACAGNFVHGHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSSSGGGGSGGGGSGGGGSGGGGSGGGSLQIYIWAPLAGTCGVLLLSLVITGSGEGRGSLLTCGDVEENPGPGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRASGGGGSGGGGSGGGGSGGGGSGGGSLQIYIWAPLAGTCGVLLLSLVIT

SEQ ID NO: 535-599 - skipped (65 entries)

SEQ ID NO: 600 - DNA, 1713 bp, synthetic construct
  source 1..1713: mol_type = other DNA
gctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgagttcctggagaacggaggaacaagcctgagcgagaaaacagtgctgctgctggtgaccccattcctggcagccgcctggagcctgcaccccggctccggcgagggcaggggaagtctactaacatgcggggacgtggaggaaaatcccggccccggaacaagcctgctgtgctggatggctctgtgcctgctgggcgctgaccacgccgacgctcagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctagagccgaattcctggagaacggagggacaagcctgagcgagaagacagtgctgctgctggtgaccccatttctggccgcagcatggagcctgcacccc

SEQ ID NO: 601 - DNA, 1716 bp, synthetic construct
  source 1..1716: mol_type = other DNA
ggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagccagggccgagttccttgaaaatggtgggacatccttatcagagaaaacagttcttctgctggtgactccatttctggcagcagcctggagccttcatcccggctccggcgccacgaacttctctctgttaaagcaagcaggagacgtggaagaaaaccccggtcccgctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgagttcctggagaacggaggaacaagcctgagcgagaaaacagtgctgctgctggtgaccccattcctggcagccgcctggagcctgcacccc

SEQ ID NO: 602 - DNA, 1194 bp, synthetic construct
  source 1..1194: mol_type = other DNA
ggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagccagggccgagttccttgaaaatggtgggacatccttatcagagaaaacagttcttctgctggtgactccatttctggcagcagcctggagccttcatcccggctccggcgccacgaacttctctctgttaaagcaagcaggagacgtggaagaaaaccccggtcccggaacaagcctgctgtgctggatggctctgtgcctgctgggcgctgaccacgccgacgctcagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctagagccgaattcctggagaacggagggacaagcctgagcgagaagacagtgctgctgctggtgaccccatttctggccgcagcatggagcctgcacccc

SEQ ID NO: 603 - protein, 571 aa, synthetic construct
AVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHPGSGEGRGSLLTCGDVEENPGPGTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRARAEFLENGGTSLSEKTVLLLVTPFLAAAWSLHP

SEQ ID NO: 604 - protein, 572 aa, synthetic construct
GTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRARAEFLENGGTSLSEKTVLLLVTPFLAAAWSLHPGSGATNFSLLKQAGDVEENPGPAVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHP

SEQ ID NO: 605 - protein, 398 aa, synthetic construct
GTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRARAEFLENGGTSLSEKTVLLLVTPFLAAAWSLHPGSGATNFSLLKQAGDVEENPGPGTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRARAEFLENGGTSLSEKTVLLLVTPFLAAAWSLHP

SEQ ID NO: 606 - DNA, 672 bp, synthetic construct
  source 1..672: mol_type = other DNA
atggccgctgagcccgtggaggacaactgcatcaacttcgtggccatgaagttcatcgacaacaccctgtactttatcgccgaggacgacgagaacctggagagcgactactttggcaagctggagagcaagctgagcgtgatccggaacctgaacgaccaggtgctgttcatcgaccagggcaatcggcctctgtttgaggacatgaccgacagcgactgcagagacaacgcacccagaaccatcttcatcatctccatgtacaaggactcccagccaaggggcatggccgtgaccatcagcgtgaaatgcgagaaaatcagcacactgtcatgcgagaacaagatcatcagcttcaaggaaatgaacccccccgacaacatcaaggacacaaaaagcgacatcatcttcttccagagatccgtccccggccacgacaacaaaatgcagttcgagagctcctcctacgagggctacttcctggcctgcgaaaaagaaagagacctgttcaagctgatcctgaagaaggaggacgagctgggcgacagaagcatcatgttcaccgtgcagaacgaggacgagttcctggagaacggggggacaagcctgtccgagaagaccgtgctgctgctggtcactcccttcctggccgccgcctggagcctgcatccc

SEQ ID NO: 607 - protein, 224 aa, synthetic construct
MAAEPVEDNCINFVAMKFIDNTLYFIAEDDENLESDYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDEFLENGGTSLSEKTVLLLVTPFLAAAWSLHP

SEQ ID NO: 608-610 - skipped (3 entries)

SEQ ID NO: 611 - DNA, 561 bp, synthetic construct
  source 1..561: mol_type = other DNA
gctgtgatggcccctagaaccctgctgctgctgctgagcggcgccctggccctgacacagacctgggccgcccctaccagcagctccaccaagaagacccagctgcagctggagcacctcctgctggacctgcagatgatcctgaacggcatcaacaactataagaatcctaagctgacaagaatgctgacctttaagttctacatgcctaagaaagctaccgagctgaagcacctgcaatgtctggaagaggagctgaaacctctggaagaggtgctgaatctggcccagagcaaaaacttccacctcagacctagagatctgatcagcaacatcaacgtgatcgtgctggaactgaagggcagcgagacaacattcatgtgcgagtacgccgacgagacagccaccattgtggaattcctgaaccggtggatcaccttctgccagtccatcatctctacactgaccgaattccttgaaaatggtgggacatccttatcagagaaaacagttcttctgctggtgactccatttctggcagcagcctggagccttcatccc

SEQ ID NO: 612 - DNA, 618 bp, synthetic construct
  source 1..618: mol_type = other DNA
gctgtgatggcccctagaaccctgctgctgctgctgagcggcgccctggccctgacacagacctgggccgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacacgaattccttgaaaatggtgggacatccttatcagagaaaacagttcttctgctggtgactccatttctggcagcagcctggagccttcatccc

SEQ ID NO: 613 - DNA, 522 bp, synthetic construct
  source 1..522: mol_type = other DNA
ctgctggccatggtgctgacaagcgccctgctgctgtgctctgtggccgggcaggggtgccctaccctggctggaatcctggacatcaacttcctgatcaacaagatgcaggaggaccccgcttccaagtgccactgctccgccaatgtgacctcctgcctgtgtctgggcattccctccgacaactgtactagaccctgcttcagcgagcgcctgagccagatgactaacaccaccatgcagacccgctaccccctgatcttctcccgcgtcaagaagtccgtcgaagtgctgaagaacaacaagtgcccctacttctcctgcgagcagccctgcaaccagaccaccgccggcaacgccctgaccttcctgaaaagcctgctggagatcttccagaaggagaaaatgcgcggcatgagaggcaagatcgagtttctggagaacggagggacaagcctgagcgagaagacagtgctgctgctggtgaccccatttctggccgcagcatggagcctgcacccc

SEQ ID NO: 614 - DNA, 1134 bp, synthetic construct
  source 1..1134: mol_type = other DNA
gctgtgatggcccctagaaccctgctgctgctgctgagcggcgccctggccctgacacagacctgggcccaccatcatcatcatcatgaattggactacaaagacgatgacgacaaggaattgatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgaattccttgaaaatggtgggacatccttatcagagaaaacagttcttctgctggtgactccatttctggcagcagcctggagccttcatccc

SEQ ID NO: 615 - DNA, 558 bp, synthetic construct
  source 1..558: mol_type = other DNA
ggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagccgaattccttgaaaatggtgggacatccttatcagagaaaacagttcttctgctggtgactccatttctggcagcagcctggagccttcatccc

SEQ ID NO: 616 - DNA, 558 bp, synthetic construct
  source 1..558: mol_type = other DNA
ggcaccagcctgctgtgctggatggcactgtgcctgctgggagcagaccacgccgatgcccagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctgaattccttgaaaatggtgggacatccttatcagagaaaacagttcttctgctggtgactccatttctggcagcagcctggagccttcatccc

SEQ ID NO: 617 - DNA, 600 bp, synthetic construct
  source 1..600: mol_type = other DNA
atgagagggacccccggggacgccgacggaggaggaagagcagtgtaccagagcatgtgcaagcccatcaccgggaccatcaacgacctgaaccagcaggtgtggaccctgcagggacagaacctggtggccgtgcccagaagcgatagcgtgacacccgtgaccgtcgccgtcattacctgcaagtaccccgaagccctggaacagggcagaggcgatcccatctacctggggattcaaaaccccgaaatgtgcctgtactgcgagaaggtcggcgagcagcctaccctgcagctgaaggaacagaaaatcatggacctgtacggacagcctgagcccgtgaaacccttcctgttctacagagctaagaccggccgcaccagcacactggagagcgtcgccttccccgactggttcatcgccagcagcaaaagagaccagcccatcatcctgacctcagagctgggaaagagctacaacaccgccttcgagctgaacatcaacgatgagttcctggagaacggggggacaagcctgtccgagaagaccgtgctgctgctggtcactcccttcctggccgccgcctggagcctgcatccc

SEQ ID NO: 618 - protein, 187 aa, synthetic construct
AVMAPRTLLLLLSGALALTQTWAAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTEFLENGGTSLSEKTVLLLVTPFLAAAWSLHP

SEQ ID NO: 619 - protein, 206 aa, synthetic construct
AVMAPRTLLLLLSGALALTQTWADCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHEFLENGGTSLSEKTVLLLVTPFLAAAWSLHP

SEQ ID NO: 620 - protein, 174 aa, synthetic construct
LLAMVLTSALLLCSVAGQGCPTLAGILDINFLINKMQEDPASKCHCSANVTSCLCLGIPSDNCTRPCFSERLSQMTNTTMQTRYPLIFSRVKKSVEVLKNNKCPYFSCEQPCNQTTAGNALTFLKSLLEIFQKEKMRGMRGKIEFLENGGTSLSEKTVLLLVTPFLAAAWSLHP

SEQ ID NO: 621 - protein, 360 aa, synthetic construct
AVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSEFLENGGTSLSEKTVLLLVTPFLAAAWSLHP

SEQ ID NO: 622 - protein, 186 aa, synthetic construct
GTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRAEFLENGGTSLSEKTVLLLVTPFLAAAWSLHP

SEQ ID NO: 623 - protein, 186 aa, synthetic construct
GTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRAEFLENGGTSLSEKTVLLLVTPFLAAAWSLHP

SEQ ID NO: 624 - protein, 200 aa, synthetic construct
MRGTPGDADGGGRAVYQSMCKPITGTINDLNQQVWTLQGQNLVAVPRSDSVTPVTVAVITCKYPEALEQGRGDPIYLGIQNPEMCLYCEKVGEQPTLQLKEQKIMDLYGQPEPVKPFLFYRAKTGRTSTLESVAFPDWFIASSKRDQPIILTSELGKSYNTAFELNINDEFLENGGTSLSEKTVLLLVTPFLAAAWSLHP

SEQ ID NO: 625 - DNA, 1762 bp, synthetic construct
  source 1..1762: mol_type = other DNA
gctgtgatggcccctagaaccctgctgctgctgctgagcggcgccctggccctgacacagacctgggccgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacacgaattccttgaaaatggtgggacatccttatcagagaaaacagttcttctgctggtgactccatttctggcagcagcctggagccttcatccctaacccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagccaccatgggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagccgagttcctggagaacggtggcaccagcctttccgagaagaccgtcttgctgctggtgactccttttctggccgcggcttggtctctccacccc

SEQ ID NO: 626 - DNA, 1731 bp, synthetic construct
  source 1..1731: mol_type = other DNA
ggaacaagcctgctgtgctggatggctctgtgcctgctgggcgctgaccacgccgacgctcagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctagagccgaattcctggagaacggagggacaagcctgagcgagaagacagtgctgctgctggtgaccccatttctggccgcagcatggagcctgcaccccgctggaacgcgtggctccggcgagggcaggggaagtcttctaacatgcggggacgtggaggaaaatcccggcccactcgaggctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgagttcctggagaacggaggaacaagcctgagcgagaaaacagtgctgctgctggtgaccccattcctggcagccgcctggagcctgcacccc

SEQ ID NO: 627 - DNA, 1209 bp, synthetic construct
  source 1..1209: mol_type = other DNA
ggaacaagcctgctgtgctggatggctctgtgcctgctgggcgctgaccacgccgacgctcagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctagagccgaattcctggagaacggagggacaagcctgagcgagaagacagtgctgctgctggtgaccccatttctggccgcagcatggagcctgcaccccgctggaacgcgtggctccggcgagggcaggggaagtcttctaacatgcggggacgtggaggaaaatcccggcccactcgagggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagccagggccgagttccttgaaaatggtgggacatccttatcagagaaaacagttcttctgctggtgactccatttctggcagcagcctggagccttcatccc

SEQ ID NO: 628 - DNA, 1731 bp, synthetic construct
  source 1..1731: mol_type = other DNA
gctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgaattcctggagaacggagggacaagcctgagcgagaagacagtgctgctgctggtgaccccatttctggccgcagcatggagcctgcaccccgctggaacgcgtggctccggcgagggcaggggaagtcttctaacatgcggggacgtggaggaaaatcccggcccactcgagggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagccagggccgagttccttgaaaatggtgggacatccttatcagagaaaacagttcttctgctggtgactccatttctggcagcagcctggagccttcatccc

SEQ ID NO: 629 - DNA, 1242 bp, synthetic construct
  source 1..1242: mol_type = other DNA
gctgtgatggcccctagaaccctgctgctgctgctgagcggcgccctggccctgacacagacctgggccgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacacgaattccttgaaaatggtgggacatccttatcagagaaaacagttcttctgctggtgactccatttctggcagcagcctggagccttcatcccggatctggagcaacaaacttctcactactcaaacaagcaggtgacgtggaggagaatccggggcccggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagccgagttcctggagaacggtggcaccagcctttccgagaagaccgtcttgctgctggtgactccttttctggccgcggcttggtctctccacccc

SEQ ID NO: 630 - DNA, 1884 bp, synthetic construct
  source 1..1884: mol_type = other DNA
gctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttctggaggaggaggatctggcggaggaggaagtggaggaggaggctctggaggaggcggatctggaggaggaagcctgcagatctacatctgggctcctctggctggcacctgcggagtgctgctgctgtctctggtgattactgggagcgggcagtgcactaactatgccctgctgaagctggccggggacgtggaatctaaccccggacctggaacctccctgctgtgttggatggccctgtgcctgctgggagctgatcacgctgacgctggaatccacgtgtttatcctgggctgctttagcgccggcctgcctaaaaccgaagccaactgggtgaacgtgatctccgacctgaagaagatcgaagacctgattcagtctatgcacatcgacgccaccctgtacaccgagagtgacgtgcacccctcctgcaaagtgacagccatgaagtgcttcctgctggagctgcaggtgatcagcctggaaagcggcgacgctagtatccacgacaccgtggagaacctgattattctggctaacaactccctgagcagtaacggaaacgtgaccgagtccggctgcaaagagtgcgaggagctggaggagaagaacattaaggagttcctgcagagcttcgtgcacatcgtgcagatgttcatcaacaccagcagagccagtggcggcggaggatctggaggcggaggaagcggaggaggaggatcaggaggaggaggaagcggcggaggatcactgcagctgctgcctagttgggccatcaccctgatcagcgtgaacggaatcttcgtgatctgttgtctgacatactgttttgctcccaggtgcagagagaggagaaggaacgagcggctgagaagagaatccgtgcggcctgtg

SEQ ID NO: 631 - DNA, 1896 bp, synthetic construct
  source 1..1896: mol_type = other DNA
gctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttctggaggaggaggatctggcggaggaggaagtggaggaggaggctctggaggaggcggatctggaggagggagtctgcagatctacatctgggctcctctggctggcacctgcggagtgctgctgctgtctctggtgattactagggccaagaggagcggaagcggggctacaaacttcagtctgctgaagcaggctggcgacgtggaggaaaaccctggacctggaaccagcctgctgtgctggatggctctgtgcctgctgggagctgaccacgctgatgctggaatccacgtgtttatcctgggatgcttctctgccgggctgcctaagaccgaggctaattgggtgaacgtgatcagcgacctgaaaaagatcgaagatctgatccagtctatgcacatcgacgccactctgtacaccgagtccgacgtgcacccctcttgcaaggtgaccgctatgaagtgcttcctgctggagctgcaggtgatcagcctggagagcggagacgctagcatccacgacacagtggaaaacctgattatcctggctaacaacagcctgagcagtaacggaaacgtgaccgagtccggctgcaaagagtgcgaggagctggaagagaagaacattaaggagttcctgcagagcttcgtgcacatcgtgcagatgttcatcaacaccagcagagccagtggcgggggaggatctggaggcggaggatctgggggaggaggaagcggaggaggagggagcggaggaggcagcctgcagctgctgccttcttgggctatcaccctgatcagcgtgaacggaatctttgtgatctgttgtctgacttattgtttcgcccccagatgcagagagaggagaaggaatgagagactgagaagagaatcagtgaggcccgtg

SEQ ID NO: 632 - DNA, 1881 bp, synthetic construct
  source 1..1881: mol_type = other DNA
gctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttctggaggaggaggatctggcggaggaggaagtggaggaggaggctctggaggaggcggatctggaggagggagtctgcagatctacatctgggctcctctggctggcacctgcggagtgctgctgctgtctctggtgattactgggtccggcgctacaaacttcagtctgctgaagcaggctggcgatgtggaggaaaaccctggccctggaacctccctgctgtgttggatggctctgtgtctgctgggggctgaccacgctgatgctggaatccacgtgttcatcctgggctgctttagcgccggcctgcctaaaaccgaggccaattgggtgaacgtgatttctgacctgaagaagatcgaggacctgattcagagtatgcatattgacgccaccctgtacaccgagagtgacgtgcaccctagctgcaaagtgacagccatgaagtgcttcctgctggagctgcaggtgatcagcctggaaagcggcgacgctagcatccacgacaccgtggaaaacctgattatcctggccaacaactccctgagcagtaacggaaacgtgaccgagtccggctgcaaagagtgcgaggagctggaagagaaaaacattaaagaattcctgcagtccttcgtgcacatcgtgcagatgttcatcaacaccagcagagccagtggcgggggaggatctggaggcggaggatctgggggaggaggaagcggaggaggagggagcggaggaggcagcctgcagctgctgccttcttgggctatcaccctgatcagcgtgaacggaatctttgtgatctgttgtctgacttattgtttcgcccccagatgcagagagaggagaaggaatgagagactgagaagagaatcagtgaggcccgtg

SEQ ID NO: 633 - DNA, 1878 bp, synthetic construct
  source 1..1878: mol_type = other DNA
gctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttctggaggaggaggatctggcggaggaggaagtggaggaggaggctctggaggaggcggatctggaggagggagtctgcagatctacatctgggctcctctggctggcacctgcggagtgctgctgctgtctctggtgattactgggtccggcgaaggaagaggctctctgctgacatgcggggatgtggaagagaatcccggccctggaacctccctgctgtgttggatggccctgtgtctgctgggggctgatcacgctgatgccggaatccacgtgtttatcctgggctgcttcagcgccgggctgcctaagacagaggctaactgggtgaacgtgatcagcgacctgaaaaagatcgaggacctgattcagagtatgcatatcgacgccaccctgtacaccgagagcgacgtgcatccttcctgcaaggtgacagccatgaagtgtttcctgctggagctgcaggtgatcagcctggaaagcggcgacgctagcatccacgacacagtggagaacctgatcatcctggccaacaacagcctgagcagcaacggaaacgtgaccgagtccggctgcaaagaatgtgaggaactggaggaaaagaacattaaggagttcctgcagagcttcgtgcacatcgtgcagatgttcatcaacaccagcagagccagtggcgggggaggatctggaggcggaggatctgggggaggaggaagcggaggaggagggagcggaggaggcagcctgcagctgctgccttcttgggctatcaccctgatcagcgtgaacggaatctttgtgatctgttgtctgacttattgtttcgcccccagatgcagagagaggagaaggaatgagagactgagaagagaatcagtgaggcccgtg

SEQ ID NO: 634 - DNA, 1884 bp, synthetic construct
  source 1..1884: mol_type = other DNA
gctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttctggaggaggaggatctggcggaggaggaagtggaggaggaggctctggaggaggcggatctggaggagggagtctgcagctgctgccaagttgggctatcacactgattagcgtgaacggaatctttgtgatctgttgcctgacatactgctttgctcctaggtgcagggagaggagaaggaatgagagactgagaagagagtccgtgaggcccgtgggaagcggacagtgtacaaactacgccctgctgaagctggccggcgatgtggaatccaaccccggacctggaacaagcctgctgtgttggatggccctgtgtctgctgggcgctgatcatgctgacgccggaatccacgtgtttatcctggggtgctttagcgccggcctgcctaaaaccgaggccaattgggtgaacgtgatcagcgacctgaaaaagatcgaggacctgattcagagtatgcatatcgacgccaccctgtacaccgagagtgacgtgcacccctcctgcaaagtgaccgctatgaagtgcttcctgctggagctgcaggtgatcagcctggaaagcggcgacgctagcatccacgacaccgtggaaaacctgatcatcctggccaacaactccctgagcagcaacggaaacgtgaccgagtccggctgcaaagaatgtgaggaactggaggaaaagaacatcaaagaattcctgcagtctttcgtgcacatcgtgcagatgttcatcaacaccagcagagccagcggcgggggaggatctggaggcggaggatctgggggaggaggaagcggaggaggagggagcggaggaggcagcctgcagatctatatttgggccccactggccggcacctgcggagtgctgctgctgtctctggtgattaca

SEQ ID NO: 635 - DNA, 1959 bp, synthetic construct
  source 1..1959: mol_type = other DNA
gctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttctggaggaggaggatctggcggaggaggaagtggaggaggaggctctggaggaggcggatctggaggagggagtctgcagctgctgccaagttgggctatcacactgattagcgtgaacggaatctttgtgatctgttgcctgacatactgctttgctcctaggtgcagggagaggagaaggaatgagagactgagaagagagtccgtgaggcccgtgggaagcggacagtgtacaaactacgccctgctgaagctggccggcgatgtggaatccaaccccggacctggaacaagcctgctgtgttggatggccctgtgtctgctgggcgctgatcatgctgacgccggaatccacgtgtttatcctggggtgctttagcgccggcctgcctaaaaccgaggccaattgggtgaacgtgatcagcgacctgaaaaagatcgaggacctgattcagagtatgcatatcgacgccaccctgtacaccgagagtgacgtgcacccctcctgcaaagtgaccgctatgaagtgcttcctgctggagctgcaggtgatcagcctggaaagcggcgacgctagcatccacgacaccgtggaaaacctgatcatcctggccaacaactccctgagcagcaacggaaacgtgaccgagtccggctgcaaagaatgtgaggaactggaggaaaagaacatcaaagaattcctgcagtctttcgtgcacatcgtgcagatgttcatcaacaccagcagagccagcggcgggggaggatctggaggcggaggatctgggggaggaggaagcggaggaggagggagcggaggaggcagcctgcagctgctgccctcttgggctatcaccctgatcagcgtgaacgggatctttgtgatttgttgtctgacttactgtttcgcccccagatgcagagagagaaggagaaacgagagactgaggagagaaagcgtgaggcccgtg

SEQ ID NO: 636 - DNA, 1878 bp, synthetic construct
  source 1..1878: mol_type = other DNA
gctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttctggaggaggaggatctggcggaggaggaagtggaggaggaggctctggaggaggcggatctggaggagggagtctgcagctgctgccaagttgggctatcacactgattagcgtgaacggaatctttgtgatctgttgcctgacatactgctttgctcctaggtgcagggagaggagaaggaatgagagactgagaagagagtccgtgaggcccgtgggaagcggagaaggaagaggaagcctgctgacctgcggggatgtggaagagaaccctggcccaggaacaagcctgctgtgctggatggccctgtgcctgctgggagctgatcatgctgacgccggaatccatgtgtttatcctgggatgcttcagcgccggactgcctaaaactgaggccaactgggtgaacgtgatttctgacctgaaaaagatcgaagacctgattcagagcatgcacatcgacgccaccctgtacactgagtccgacgtgcacccctcctgcaaagtgacagccatgaagtgtttcctgctggagctgcaggtgatcagcctggagagcggagacgctagcatccacgacacagtggaaaacctgatcatcctggctaacaacagcctgagcagcaacggaaacgtgaccgagtccggctgcaaagagtgcgaggagctggaggagaagaacattaaggagttcctgcagagcttcgtgcacatcgtgcagatgttcatcaacaccagcagagccagtggcgggggaggatctggaggcggaggatctgggggaggaggaagcggaggaggagggagcggaggaggcagcctgcagatctatatttgggcccccctggccggcacatgcggagtgctgctgctgtctctggtgattaca

SEQ ID NO: 637 - DNA, 1953 bp, synthetic construct
  source 1..1953: mol_type = other DNA
gctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttctggaggaggaggatctggcggaggaggaagtggaggaggaggctctggaggaggcggatctggaggagggagtctgcagctgctgccaagttgggctatcacactgattagcgtgaacggaatctttgtgatctgttgcctgacatactgctttgctcctaggtgcagggagaggagaaggaatgagagactgagaagagagtccgtgaggcccgtgggaagcggagaaggaagaggaagcctgctgacctgcggggatgtggaagagaaccctggcccaggaacaagcctgctgtgctggatggccctgtgcctgctgggagctgatcatgctgacgccggaatccatgtgtttatcctgggatgcttcagcgccggactgcctaaaactgaggccaactgggtgaacgtgatttctgacctgaaaaagatcgaagacctgattcagagcatgcacatcgacgccaccctgtacactgagtccgacgtgcacccctcctgcaaagtgacagccatgaagtgtttcctgctggagctgcaggtgatcagcctggagagcggagacgctagcatccacgacacagtggaaaacctgatcatcctggctaacaacagcctgagcagcaacggaaacgtgaccgagtccggctgcaaagagtgcgaggagctggaggagaagaacattaaggagttcctgcagagcttcgtgcacatcgtgcagatgttcatcaacaccagcagagccagtggcgggggaggatctggaggcggaggatctgggggaggaggaagcggaggaggagggagcggaggaggcagcctgcagctgctgccctcttgggctatcaccctgatcagcgtgaacgggatctttgtgatttgttgtctgacttactgtttcgcccccagatgcagagagagaaggagaaacgagagactgaggagagaaagcgtgaggcccgtg

SEQ ID NO: 638 - DNA, 1947 bp, synthetic construct
  source 1..1947: mol_type = other DNA
gctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttctggaggaggaggatctggcggaggaggaagtggaggaggaggctctggaggaggcggatctggaggagggggatctggaggcggaggatctggagggggaggatctgggggaggaggaagcggaggaggagggtctctgcagatctacatctgggctcctctggctggaacatgtggcgtgctgctgctgagcctggtgatcacaggctctgggcagtgtaccaattatgctctgctgaagctggctggggacgtggaaagcaaccccggacctggaacctctctgctgtgctggatggccctgtgcctgctgggagctgatcacgctgatgccggaatccacgtgttcattctgggctgcttctctgccggcctgccaaaaactgaggctaactgggtgaatgtgattagtgacctgaaaaagatcgaggacctgatccagagtatgcatatcgacgccaccctgtacaccgaatccgacgtgcacccctcctgcaaagtgaccgctatgaagtgtttcctgctggagctgcaggtgatcagcctggagagcggagacgctagcatccacgacaccgtggaaaacctgatcatcctggccaacaacagcctgagcagcaacggcaacgtgaccgagtccggatgcaaagagtgcgaggagctggaggagaagaacattaaagagttcctgcagagcttcgtgcacatcgtgcagatgttcatcaacaccagcagagccagcggcggcggaggaagcggcggaggaggcagcggaggaggcggctctggaggcggcggatctggcggcggaagcctgcagctgctgccttcttgggctatcaccctgatcagcgtgaacggaatttttgtgatctgttgtctgacttactgttttgctccccggtgtagggagaggagaagaaacgagagactgagaagagaatcagtgaggcccgtg

SEQ ID NO: 639 - protein, 577 aa, synthetic construct
GTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRARAEFLENGGTSLSEKTVLLLVTPFLAAAWSLHPAGTRGSGEGRGSLLTCGDVEENPGPLEAVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHP

SEQ ID NO: 640 - protein, 403 aa, synthetic construct
GTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRARAEFLENGGTSLSEKTVLLLVTPFLAAAWSLHPAGTRGSGEGRGSLLTCGDVEENPGPLEGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRARAEFLENGGTSLSEKTVLLLVTPFLAAAWSLHP

SEQ ID NO: 641 - protein, 577 aa, synthetic construct
AVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHPAGTRGSGEGRGSLLTCGDVEENPGPLEGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRARAEFLENGGTSLSEKTVLLLVTPFLAAAWSLHP

SEQ ID NO: 642 - protein, 414 aa, synthetic construct
AVMAPRTLLLLLSGALALTQTWADCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHEFLENGGTSLSEKTVLLLVTPFLAAAWSLHPGSGATNFSLLKQAGDVEENPGPGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRAEFLENGGTSLSEKTVLLLVTPFLAAAWSLHP

SEQ ID NO: 643 - protein, 628 aa, synthetic construct
AVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSGGGGSGGGGSGGGGSGGGGSGGGSLQIYIWAPLAGTCGVLLLSLVITGSGQCTNYALLKLAGDVESNPGPGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRASGGGGSGGGGSGGGGSGGGGSGGGSLQLLPSWAITLISVNGIFVICCLTYCFAPRCRERRRNERLRRESVRPV

SEQ ID NO: 644 - protein, 632 aa, synthetic construct
AVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSGGGGSGGGGSGGGGSGGGGSGGGSLQIYIWAPLAGTCGVLLLSLVITRAKRSGSGATNFSLLKQAGDVEENPGPGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRASGGGGSGGGGSGGGGSGGGGSGGGSLQLLPSWAITLISVNGIFVICCLTYCFAPRCRERRRNERLRRESVRPV

SEQ ID NO: 645 - protein, 627 aa, synthetic construct
AVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSGGGGSGGGGSGGGGSGGGGSGGGSLQIYIWAPLAGTCGVLLLSLVITGSGATNFSLLKQAGDVEENPGPGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRASGGGGSGGGGSGGGGSGGGGSGGGSLQLLPSWAITLISVNGIFVICCLTYCFAPRCRERRRNERLRRESVRPV

SEQ ID NO: 646 - protein, 626 aa, synthetic construct
AVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSGGGGSGGGGSGGGGSGGGGSGGGSLQIYIWAPLAGTCGVLLLSLVITGSGEGRGSLLTCGDVEENPGPGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRASGGGGSGGGGSGGGGSGGGGSGGGSLQLLPSWAITLISVNGIFVICCLTYCFAPRCRERRRNERLRRESVRPV

SEQ ID NO: 647 - protein, 628 aa, synthetic construct
AVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSGGGGSGGGGSGGGGSGGGGSGGGSLQLLPSWAITLISVNGIFVICCLTYCFAPRCRERRRNERLRRESVRPVGSGQCTNYALLKLAGDVESNPGPGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRASGGGGSGGGGSGGGGSGGGGSGGGSLQIYIWAPLAGTCGVLLLSLVIT

SEQ ID NO: 648 - protein, 653 aa, synthetic construct
AVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSGGGGSGGGGSGGGGSGGGGSGGGSLQLLPSWAITLISVNGIFVICCLTYCFAPRCRERRRNERLRRESVRPVGSGQCTNYALLKLAGDVESNPGPGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRASGGGGSGGGGSGGGGSGGGGSGGGSLQLLPSWAITLISVNGIFVICCLTYCFAPRCRERRRNERLRRESVRPV

SEQ ID NO: 649 - protein, 626 aa, synthetic construct
AVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSGGGGSGGGGSGGGGSGGGGSGGGSLQLLPSWAITLISVNGIFVICCLTYCFAPRCRERRRNERLRRESVRPVGSGEGRGSLLTCGDVEENPGPGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRASGGGGSGGGGSGGGGSGGGGSGGGSLQIYIWAPLAGTCGVLLLSLVIT

SEQ ID NO: 650 - protein, 651 aa, synthetic construct
AVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSGGGGSGGGGSGGGGSGGGGSGGGSLQLLPSWAITLISVNGIFVICCLTYCFAPRCRERRRNERLRRESVRPVGSGEGRGSLLTCGDVEENPGPGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRASGGGGSGGGGSGGGGSGGGGSGGGSLQLLPSWAITLISVNGIFVICCLTYCFAPRCRERRRNERLRRESVRPV

SEQ ID NO: 651 - protein, 649 aa, synthetic construct
AVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSSGGGGSGGGGSGGGGSGGGGSGGGGSGGGGSGGGGSGGGGSGGGGSLQIYIWAPLAGTCGVLLLSLVITGSGQCTNYALLKLAGDVESNPGPGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRASGGGGSGGGGSGGGGSGGGGSGGGSLQLLPSWAITLISVNGIFVICCLTYCFAPRCRERRRNERLRRESVRPV

SEQ ID NO: 652 - DNA, 2379 bp, synthetic construct
  source 1..2379: mol_type = other DNA
ggaacaagcctgctgtgctggatggctctgtgcctgctgggcgctgaccacgccgacgctcagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctagagccgaattcctggagaacggagggacaagcctgagcgagaagacagtgctgctgctggtgaccccatttctggccgcagcatggagcctgcaccccgctggaacgcgtggctccggcgagggcaggggaagtcttctaacatgcggggacgtggaggaaaatcccggcccagctagcgctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgagttcctggagaacggaggaacaagcctgagcgagaaaacagtgctgctgctggtgaccccattcctggcagccgcctggagcctgcaccccgctggaatcgatggcagcggcgccacaaacttctctctgctaaagcaagcaggtgatgttgaagaaaaccccgggcctctcgagggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagccagggccgagttccttgaaaatggtgggacatccttatcagagaaaacagttcttctgctggtgactccatttctggcagcagcctggagccttcatccc

SEQ ID NO: 653 - DNA, 2379 bp, synthetic construct
  source 1..2379: mol_type = other DNA
gctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgagttcctggagaacggaggaacaagcctgagcgagaaaacagtgctgctgctggtgaccccattcctggcagccgcctggagcctgcaccccgctggaacgcgtggctccggcgagggcaggggaagtcttctaacatgcggggacgtggaggaaaatcccggcccagctagcggaacaagcctgctgtgctggatggctctgtgcctgctgggcgctgaccacgccgacgctcagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctagagccgaattcctggagaacggagggacaagcctgagcgagaagacagtgctgctgctggtgaccccatttctggccgcagcatggagcctgcaccccgctggaatcgatggcagcggcgccacaaacttctctctgctaaagcaagcaggtgatgttgaagaaaaccccgggcctctcgagggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagccagggccgagttccttgaaaatggtgggacatccttatcagagaaaacagttcttctgctggtgactccatttctggcagcagcctggagccttcatccc

SEQ ID NO: 654 - DNA, 2379 bp, synthetic construct
  source 1..2379: mol_type = other DNA
ggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagccagggccgagttccttgaaaatggtgggacatccttatcagagaaaacagttcttctgctggtgactccatttctggcagcagcctggagccttcatcccgctggaacgcgtggctccggcgagggcaggggaagtcttctaacatgcggggacgtggaggaaaatcccggcccagctagcggaacaagcctgctgtgctggatggctctgtgcctgctgggcgctgaccacgccgacgctcagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctagagccgaattcctggagaacggagggacaagcctgagcgagaagacagtgctgctgctggtgaccccatttctggccgcagcatggagcctgcaccccgctggaatcgatggcagcggcgccacaaacttctctctgctaaagcaagcaggtgatgttgaagaaaaccccgggcctctcgaggctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgagttcctggagaacggaggaacaagcctgagcgagaaaacagtgctgctgctggtgaccccattcctggcagccgcctggagcctgcacccc

SEQ ID NO: 655 - protein, 793 aa, synthetic construct
GTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRARAEFLENGGTSLSEKTVLLLVTPFLAAAWSLHPAGTRGSGEGRGSLLTCGDVEENPGPASAVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHPAGIDGSGATNFSLLKQAGDVEENPGPLEGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRARAEFLENGGTSLSEKTVLLLVTPFLAAAWSLHP

SEQ ID NO: 656 - protein, 793 aa, synthetic construct
AVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHPAGTRGSGEGRGSLLTCGDVEENPGPASGTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRARAEFLENGGTSLSEKTVLLLVTPFLAAAWSLHPAGIDGSGATNFSLLKQAGDVEENPGPLEGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRARAEFLENGGTSLSEKTVLLLVTPFLAAAWSLHP

SEQ ID NO: 657 - protein, 793 aa, synthetic construct
GTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRARAEFLENGGTSLSEKTVLLLVTPFLAAAWSLHPAGTRGSGEGRGSLLTCGDVEENPGPASGTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRARAEFLENGGTSLSEKTVLLLVTPFLAAAWSLHPAGIDGSGATNFSLLKQAGDVEENPGPLEAVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHP

SEQ ID NO: 658 - DNA, 2079 bp, synthetic construct
  source 1..2079: mol_type = other DNA
ggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagccgaattccttgaaaatggtgggacatccttatcagagaaaacagttcttctgctggtgactccatttctggcagcagcctggagccttcatcccggctccggcgccacgaacttctctctgttaaagcaagcaggagacgtggaagaaaaccccggtcccgccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 659 - DNA, 2604 bp, synthetic construct
  source 1..2604: mol_type = other DNA
gctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgagttcctggagaacggaggaacaagcctgagcgagaaaacagtgctgctgctggtgaccccattcctggcagccgcctggagcctgcaccccggaagtggagccacgaacttctctctgttaaagcaagcaggagacgtggaagaaaaccccggtccctctcgctccgtggccttagctgtgctcgcgctactctctctttctggcctggaggctgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 660 - DNA, 2079 bp, synthetic construct
  source 1..2079: mol_type = other DNA
ggcaccagcctgctgtgctggatggcactgtgcctgctgggagcagaccacgccgatgcccagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctgaattccttgaaaatggtgggacatccttatcagagaaaacagttcttctgctggtgactccatttctggcagcagcctggagccttcatcccggatctggagcaacaaacttctcactactcaaacaagcaggtgacgtggaggagaatccggggcccgccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 661 - DNA, 2601 bp, synthetic construct
  source 1..2601: mol_type = other DNA
gctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgagttcctggagaacggaggaacaagcctgagcgagaaaacagtgctgctgctggtgaccccattcctggcagccgcctggagcctgcaccccggaagtggagagggcaggggaagtctactaacatgcggggacgtggaggaaaatcccggcccctctcgctccgtggccttagctgtgctcgcgctactctctctttctggcctggaggctgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 662 - DNA, 2076 bp, synthetic construct
  source 1..2076: mol_type = other DNA
ggcaccagcctgctgtgctggatggcactgtgcctgctgggagcagaccacgccgatgcccagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctgaattccttgaaaatggtgggacatccttatcagagaaaacagttcttctgctggtgactccatttctggcagcagcctggagccttcatcccggctccggcgagggcaggggaagtctactaacatgcggggacgtggaggaaaatcccggccccgccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 663 - DNA, 2079 bp, synthetic construct
  source 1..2079: mol_type = other DNA
gccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctccggcgccacgaacttctctctgttaaagcaagcaggagacgtggaagaaaaccccggtcccggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagccgaattccttgaaaatggtgggacatccttatcagagaaaacagttcttctgctggtgactccatttctggcagcagcctggagccttcatccc

SEQ ID NO: 664 - DNA, 2655 bp, synthetic construct
  source 1..2655: mol_type = other DNA
gccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggatctggagcaacaaacttctcactactcaaacaagcaggtgacgtggaggagaatccggggcccgctgtgatggcccctagaaccctgctgctgctgctgagcggcgccctggccctgacacagacctgggcccaccatcatcatcatcatgaattggactacaaagacgatgacgacaaggaattgatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgaattccttgaaaatggtgggacatccttatcagagaaaacagttcttctgctggtgactccatttctggcagcagcctggagccttcatccc

SEQ ID NO: 665 - DNA, 2652 bp, synthetic construct
  source 1..2652: mol_type = other DNA
gccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctccggcgagggcaggggaagtctactaacatgcggggacgtggaggaaaatcccggccccgctgtgatggcccctagaaccctgctgctgctgctgagcggcgccctggccctgacacagacctgggcccaccatcatcatcatcatgaattggactacaaagacgatgacgacaaggaattgatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtgaattccttgaaaatggtgggacatccttatcagagaaaacagttcttctgctggtgactccatttctggcagcagcctggagccttcatccc

SEQ ID NO: 666 - DNA, 2076 bp, synthetic construct
  source 1..2076: mol_type = other DNA
gccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggctccggcgagggcaggggaagtctactaacatgcggggacgtggaggaaaatcccggccccggcaccagcctgctgtgctggatggcactgtgcctgctgggagcagaccacgccgatgcccagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctgaattccttgaaaatggtgggacatccttatcagagaaaacagttcttctgctggtgactccatttctggcagcagcctggagccttcatccc

SEQ ID NO: 667 - protein, 693 aa, synthetic construct
GTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRAEFLENGGTSLSEKTVLLLVTPFLAAAWSLHPGSGATNFSLLKQAGDVEENPGPALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 668 - protein, 868 aa, synthetic construct
AVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHPGSGATNFSLLKQAGDVEENPGPSRSVALAVLALLSLSGLEADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 669 - protein, 693 aa, synthetic construct
GTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRAEFLENGGTSLSEKTVLLLVTPFLAAAWSLHPGSGATNFSLLKQAGDVEENPGPALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 670 - protein, 867 aa, synthetic construct
AVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHPGSGEGRGSLLTCGDVEENPGPSRSVALAVLALLSLSGLEADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 671 - protein, 692 aa, synthetic construct
GTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRAEFLENGGTSLSEKTVLLLVTPFLAAAWSLHPGSGEGRGSLLTCGDVEENPGPALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 672 - protein, 693 aa, synthetic construct
ALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRAEFLENGGTSLSEKTVLLLVTPFLAAAWSLHP

SEQ ID NO: 673 - protein, 885 aa, synthetic construct
ALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPAVMAPRTLLLLLSGALALTQTWAHHHHHHELDYKDDDDKELIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSEFLENGGTSLSEKTVLLLVTPFLAAAWSLHP

SEQ ID NO: 674 - protein, 884 aa, synthetic construct
ALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPAVMAPRTLLLLLSGALALTQTWAHHHHHHELDYKDDDDKELIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSEFLENGGTSLSEKTVLLLVTPFLAAAWSLHP

SEQ ID NO: 675 - protein, 692 aa, synthetic construct
ALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPGTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRAEFLENGGTSLSEKTVLLLVTPFLAAAWSLHP

SEQ ID NO: 676 - DNA, 3225 bp, synthetic construct
  source 1..3225: mol_type = other DNA
tctcgctccgtggccttagctgtgctcgcgctactctctctttctggcctggaggctgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagtggagagggcaggggaagtctactaacatgcggggacgtggaggaaaatcccggccccggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagccgaattccttgaaaatggtgggacatccttatcagagaaaacagttcttctgctggtgactccatttctggcagcagcctggagccttcatcccggctccggcgccacgaacttctctctgttaaagcaagcaggagacgtggaagaaaaccccggtcccgctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgagttcctggagaacggaggaacaagcctgagcgagaaaacagtgctgctgctggtgaccccattcctggcagccgcctggagcctgcacccc

SEQ ID NO: 677 - DNA, 3270 bp, synthetic construct
  source 1..3270: mol_type = other DNA
gccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctggaacgcgtggctccggcgagggcaggggaagtcttctaacatgcggggacgtggaggaaaatcccggcccagctagcggaacaagcctgctgtgctggatggctctgtgcctgctgggcgctgaccacgccgacgctcagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctagagccgaattcctggagaacggagggacaagcctgagcgagaagacagtgctgctgctggtgaccccatttctggccgcagcatggagcctgcaccccgctggaatcgatggcagcggcgccacaaacttctctctgctaaagcaagcaggtgatgttgaagaaaaccccgggcctctcgaggctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgagttcctggagaacggaggaacaagcctgagcgagaaaacagtgctgctgctggtgaccccattcctggcagccgcctggagcctgcacccc

SEQ ID NO: 678 - DNA, 3225 bp, synthetic construct
  source 1..3225: mol_type = other DNA
ggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagccgaattccttgaaaatggtgggacatccttatcagagaaaacagttcttctgctggtgactccatttctggcagcagcctggagccttcatcccggctccggcgccacgaacttctctctgttaaagcaagcaggagacgtggaagaaaaccccggtcccgctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgagttcctggagaacggaggaacaagcctgagcgagaaaacagtgctgctgctggtgaccccattcctggcagccgcctggagcctgcaccccggaagtggagagggcaggggaagtctactaacatgcggggacgtggaggaaaatcccggcccctctcgctccgtggccttagctgtgctcgcgctactctctctttctggcctggaggctgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 679 - DNA, 2712 bp, synthetic construct
  source 1..2712: mol_type = other DNA
ggaacaagcctgctgtgctggatggctctgtgcctgctgggcgctgaccacgccgacgctggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagccagagccgagtttctggagaacggagggacaagcctgagcgagaagacagtgctgctgctggtgaccccatttctggccgcagcatggagcctgcaccccggctccggcgccacgaacttctctctgttaaagcaagcaggagacgtggaagaaaaccccggtcccggcaccagcctgctgtgctggatggcactgtgcctgctgggagcagaccacgccgatgcccagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctagagccgaattccttgaaaatggtgggacatccttatcagagaaaacagttcttctgctggtgactccatttctggcagcagcctggagccttcatcccggctccggcgagggcaggggaagtctactaacatgcggggacgtggaggaaaatcccggccccgccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 680 - DNA, 2712 bp, synthetic construct
  source 1..2712: mol_type = other DNA
ggaacaagcctgctgtgctggatggctctgtgcctgctgggcgctgaccacgccgacgctcagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctagagccgagtttctggagaacggagggacaagcctgagcgagaagacagtgctgctgctggtgaccccatttctggccgcagcatggagcctgcaccccggatctggagcaacaaacttctcactactcaaacaagcaggtgacgtggaggagaatccggggcccggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagccagagccgaattccttgaaaatggtgggacatccttatcagagaaaacagttcttctgctggtgactccatttctggcagcagcctggagccttcatcccggctccggcgagggcaggggaagtctactaacatgcggggacgtggaggaaaatcccggccccgccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 681 - DNA, 3234 bp, synthetic construct
  source 1..3234: mol_type = other DNA
gctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgagttcctggagaacggaggaacaagcctgagcgagaaaacagtgctgctgctggtgaccccattcctggcagccgcctggagcctgcaccccggaagtggagccacgaacttctctctgttaaagcaagcaggagacgtggaagaaaaccccggtcccggcaccagcctgctgtgctggatggcactgtgcctgctgggagcagaccacgccgatgcccagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctagagccgaattccttgaaaatggtgggacatccttatcagagaaaacagttcttctgctggtgactccatttctggcagcagcctggagccttcatcccggctccggcgagggcaggggaagtctactaacatgcggggacgtggaggaaaatcccggccccgccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 682 - DNA, 3234 bp, synthetic construct
  source 1..3234: mol_type = other DNA
ggcaccagcctgctgtgctggatggcactgtgcctgctgggagcagaccacgccgatgcccagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctagagccgaattccttgaaaatggtgggacatccttatcagagaaaacagttcttctgctggtgactccatttctggcagcagcctggagccttcatcccggatctggagcaacaaacttctcactactcaaacaagcaggtgacgtggaggagaatccggggcccgctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgagttcctggagaacggaggaacaagcctgagcgagaaaacagtgctgctgctggtgaccccattcctggcagccgcctggagcctgcaccccggaagtggagagggcaggggaagtctactaacatgcggggacgtggaggaaaatcccggccccgccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 683 - DNA, 3270 bp, synthetic construct
  source 1..3270: mol_type = other DNA
gctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgagttcctggagaacggaggaacaagcctgagcgagaaaacagtgctgctgctggtgaccccattcctggcagccgcctggagcctgcaccccgctggaacgcgtggctccggcgagggcaggggaagtcttctaacatgcggggacgtggaggaaaatcccggcccagctagcggaacaagcctgctgtgctggatggctctgtgcctgctgggcgctgaccacgccgacgctcagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctagagccgaattcctggagaacggagggacaagcctgagcgagaagacagtgctgctgctggtgaccccatttctggccgcagcatggagcctgcaccccgctggaatcgatggcagcggcgccacaaacttctctctgctaaagcaagcaggtgatgttgaagaaaaccccgggcctctcgaggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 684 - DNA, 3270 bp, synthetic construct
  source 1..3270: mol_type = other DNA
ggaacaagcctgctgtgctggatggctctgtgcctgctgggcgctgaccacgccgacgctcagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctagagccgaattcctggagaacggagggacaagcctgagcgagaagacagtgctgctgctggtgaccccatttctggccgcagcatggagcctgcaccccgctggaacgcgtggctccggcgagggcaggggaagtcttctaacatgcggggacgtggaggaaaatcccggcccagctagcgctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgagttcctggagaacggaggaacaagcctgagcgagaaaacagtgctgctgctggtgaccccattcctggcagccgcctggagcctgcaccccgctggaatcgatggcagcggcgccacaaacttctctctgctaaagcaagcaggtgatgttgaagaaaaccccgggcctctcgaggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 685 - DNA, 3270 bp, synthetic construct
  source 1..3270: mol_type = other DNA
gctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgagttcctggagaacggaggaacaagcctgagcgagaaaacagtgctgctgctggtgaccccattcctggcagccgcctggagcctgcaccccgctggaacgcgtggctccggcgagggcaggggaagtcttctaacatgcggggacgtggaggaaaatcccggcccagctagcgccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctggaatcgatggcagcggcgccacaaacttctctctgctaaagcaagcaggtgatgttgaagaaaaccccgggcctctcgagggaacaagcctgctgtgctggatggctctgtgcctgctgggcgctgaccacgccgacgctcagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctagagccgaattcctggagaacggagggacaagcctgagcgagaagacagtgctgctgctggtgaccccatttctggccgcagcatggagcctgcacccc

SEQ ID NO: 686 - DNA, 3270 bp, synthetic construct
  source 1..3270: mol_type = other DNA
ggaacaagcctgctgtgctggatggctctgtgcctgctgggcgctgaccacgccgacgctcagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctagagccgaattcctggagaacggagggacaagcctgagcgagaagacagtgctgctgctggtgaccccatttctggccgcagcatggagcctgcaccccgctggaacgcgtggctccggcgagggcaggggaagtcttctaacatgcggggacgtggaggaaaatcccggcccagctagcgccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgctggaatcgatggcagcggcgccacaaacttctctctgctaaagcaagcaggtgatgttgaagaaaaccccgggcctctcgaggctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgagttcctggagaacggaggaacaagcctgagcgagaaaacagtgctgctgctggtgaccccattcctggcagccgcctggagcctgcacccc

SEQ ID NO: 687 - protein, 1075 aa, synthetic construct
SRSVALAVLALLSLSGLEADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRAEFLENGGTSLSEKTVLLLVTPFLAAAWSLHPGSGATNFSLLKQAGDVEENPGPAVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHP

SEQ ID NO: 688 - protein, 1090 aa, synthetic construct
ALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAGTRGSGEGRGSLLTCGDVEENPGPASGTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRARAEFLENGGTSLSEKTVLLLVTPFLAAAWSLHPAGIDGSGATNFSLLKQAGDVEENPGPLEAVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHP

SEQ ID NO: 689 - protein, 1075 aa, synthetic construct
GTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRAEFLENGGTSLSEKTVLLLVTPFLAAAWSLHPGSGATNFSLLKQAGDVEENPGPAVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHPGSGEGRGSLLTCGDVEENPGPSRSVALAVLALLSLSGLEADIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 690 - protein, 904 aa, synthetic construct
GTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRARAEFLENGGTSLSEKTVLLLVTPFLAAAWSLHPGSGATNFSLLKQAGDVEENPGPGTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRARAEFLENGGTSLSEKTVLLLVTPFLAAAWSLHPGSGEGRGSLLTCGDVEENPGPALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 691 - protein, 904 aa, synthetic construct
GTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRARAEFLENGGTSLSEKTVLLLVTPFLAAAWSLHPGSGATNFSLLKQAGDVEENPGPGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRARAEFLENGGTSLSEKTVLLLVTPFLAAAWSLHPGSGEGRGSLLTCGDVEENPGPALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 692 - protein, 1078 aa, synthetic construct
AVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHPGSGATNFSLLKQAGDVEENPGPGTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRARAEFLENGGTSLSEKTVLLLVTPFLAAAWSLHPGSGEGRGSLLTCGDVEENPGPALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 693 - protein, 1078 aa, synthetic construct
GTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRARAEFLENGGTSLSEKTVLLLVTPFLAAAWSLHPGSGATNFSLLKQAGDVEENPGPAVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHPGSGEGRGSLLTCGDVEENPGPALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 694 - protein, 1090 aa, synthetic construct
AVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHPAGTRGSGEGRGSLLTCGDVEENPGPASGTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRARAEFLENGGTSLSEKTVLLLVTPFLAAAWSLHPAGIDGSGATNFSLLKQAGDVEENPGPLEALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 695 - protein, 1090 aa, synthetic construct
GTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRARAEFLENGGTSLSEKTVLLLVTPFLAAAWSLHPAGTRGSGEGRGSLLTCGDVEENPGPASAVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHPAGIDGSGATNFSLLKQAGDVEENPGPLEALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 696 - protein, 1090 aa, synthetic construct
AVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHPAGTRGSGEGRGSLLTCGDVEENPGPASALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAGIDGSGATNFSLLKQAGDVEENPGPLEGTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRARAEFLENGGTSLSEKTVLLLVTPFLAAAWSLHP

SEQ ID NO: 697 - protein, 1090 aa, synthetic construct
GTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRARAEFLENGGTSLSEKTVLLLVTPFLAAAWSLHPAGTRGSGEGRGSLLTCGDVEENPGPASALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRAGIDGSGATNFSLLKQAGDVEENPGPLEAVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHP

SEQ ID NO: 698 - DNA, 4695 bp, synthetic construct
  source 1..4695: mol_type = other DNA
ggaacaagcctgctgtgctggatggctctgtgcctgctgggcgctgaccacgccgacgctcagggccaggacagacacatgatcagaatgagacagctgatcgacatcgtggaccagctgaagaactacgtgaacgacctggtgcccgagttcctgcccgctcctgaagacgtggaaacaaactgtgagtggagcgctttcagctgcttccagaaggcccagctgaagagcgccaataccggaaacaatgagagaatcatcaacgtgagcatcaagaagctgaagagaaagccccccagcaccaacgccggaagaagacaaaaacatagactgacctgccccagctgcgatagctacgagaaaaagccccccaaggagttcctggagagattcaagagcctgctgcagaagatgatccaccagcacctgagcagcagaacccacggctctgaagatagcagagctagagccgaattcctggagaacggagggacaagcctgagcgagaagacagtgctgctgctggtgaccccatttctggccgcagcatggagcctgcaccccgctggaacgcgtggctccggcgagggcaggggaagtcttctaacatgcggggacgtggaggaaaatcccggcccagctagcgctgtgatggcccctagaaccctgctgctgctgctgagcggcgccctggccctgacacagacctgggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtagagccgagttcctggagaacggaggaacaagcctgagcgagaaaacagtgctgctgctggtgaccccattcctggcagccgcctggagcctgcaccccgctggaatcgatggcagcggcgccacaaacttctctctgctaaagcaagcaggtgatgttgaagaaaaccccgggcctctcgagggcaccagcctgctgtgctggatggcactgtgcctgctgggagcagaccacgccgatgccggcatccacgtgttcatcctgggctgcttcagcgccggacttcctaaaacagaggccaactgggtgaacgtgattagcgacctgaagaagatcgaggacctgatccagagcatgcacatcgacgccaccctgtacaccgagagcgatgtgcatcctagctgcaaggtgaccgccatgaagtgcttcctgctggagctgcaggtgatcagcctggagagcggagatgccagcattcacgacacagtggaaaatctgatcatcctggccaacaacagcctgagcagcaacggcaatgtgaccgagagcggctgtaaggagtgcgaggaactggaggagaagaacatcaaggagttcctgcagagcttcgtgcacatcgtgcagatgttcatcaacaccagcagagccagggccgagttccttgaaaatggtgggacatccttatcagagaaaacagttcttctgctggtgactccatttctggcagcagcctggagccttcatcccggctccggcgagggcaggggaagtctactaacatgcggggacgtggaggaaaatcccggccccgccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 699 - protein, 1299 aa, synthetic construct
GTSLLCWMALCLLGADHADAQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSRARAEFLENGGTSLSEKTVLLLVTPFLAAAWSLHPAGTRGSGEGRGSLLTCGDVEENPGPASAVMAPRTLLLLLSGALALTQTWAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSRAEFLENGGTSLSEKTVLLLVTPFLAAAWSLHPAGIDGSGATNFSLLKQAGDVEENPGPLEGTSLLCWMALCLLGADHADAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSRARAEFLENGGTSLSEKTVLLLVTPFLAAAWSLHPGSGEGRGSLLTCGDVEENPGPALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 700 - protein, 50 aa, synthetic construct
  VARIANT 1..5: note = Residue may be deleted
  VARIANT 6..10: note = Residue may be deleted
  VARIANT 11..15: note = Residue may be deleted
  VARIANT 16..20: note = Residue may be deleted
  VARIANT 21..25: note = Residue may be deleted
  VARIANT 26..30: note = Residue may be deleted
  VARIANT 31..35: note = Residue may be deleted
  VARIANT 36..40: note = Residue may be deleted
  VARIANT 41..45: note = Residue may be deleted
  VARIANT 46..50: note = Residue may be deleted
  VARIANT 1..50: note = (G1-5S1-5) repeats may be deleted
GGGGGSSSSSGGGGGSSSSSGGGGGSSSSSGGGGGSSSSSGGGGGSSSSS

SEQ ID NO: 701 - protein, 5 aa, synthetic construct
DGGGS

SEQ ID NO: 702 - protein, 5 aa, synthetic construct
TGEKP

SEQ ID NO: 703 - protein, 25 aa, synthetic construct
  VARIANT 6..25: note = GGGGS repeats may be deleted
GGGGSGGGGSGGGGSGGGGSGGGGS

SEQ ID NO: 704 - protein, 14 aa, synthetic construct
EGKSSGSGSESKVD

SEQ ID NO: 705 - protein, 18 aa, synthetic construct
KESGSVSSEQLAQFRSLD

SEQ ID NO: 706 - protein, 8 aa, synthetic construct
GGRRGGGS

SEQ ID NO: 707 - protein, 9 aa, synthetic construct
LRQRDGERP

SEQ ID NO: 708 - protein, 12 aa, synthetic construct
LRQKDGGGSERP

SEQ ID NO: 709 - protein, 16 aa, synthetic construct
LRQKDGGGSGGGSERP

SEQ ID NO: 710 - protein, 5 aa, synthetic construct
GGGGS

SEQ ID NO: 711 - protein, 4 aa, synthetic construct
GGGS